(19) United States
(12) Patent Application Publication
(10) Pub. No.: US 2004/0067882 A1
(43) Pub. Date: Apr. 8, 2004

(54) Therapeutic polypeptides, nucleic acids encoding same, and methods of use

(76) Inventors: John P. Alsobrook II, Madison, CT (US); Enrique Alvarez, Clinton, CT (US); David W. Anderson, Branford, CT (US); Melanie Baron, Branford, CT (US); Ferenc L. Boldog, North Haven, CT (US); Catherine E. Burgess, Wethersfield, CT (US); Stacie J. Casman, North Haven, CT (US); Andrei Chapoval, Branford, CT (US); Mohanraj Dhanabal, Branford, CT (US); Shlomit R. Edinger, New Haven, CT (US); Andrew Eisen, Rockville, MD (US); Karen Ellerman, Branford, CT (US); Seth Ettenberg, New Haven, CT (US); Esha A. Gangolli, Madison, CT (US); Valerie Gerlach, Branford, CT (US); Linda Gorman, Branford, CT (US); William M. Grosse, Branford, CT (US); Xiaojia (Sasha) Guo, Branford, CT (US); Craig Hackett, Branford, CT (US); Weizhen Ji, Branford, CT (US); Ramesh Kekuda, Norwalk, CT (US); Nikolai V. Khramtsov, Branford, CT (US); Denise M. Lepley, Farmington, CT (US); Li Li, Branford, CT (US); John R. MacDougall, Hamden, CT (US); Uriel M. Malyankar, Branford, CT (US); Ann Mazur, Bloomfield, CT (US); Kelly McQueeney, Ansonia, CT (US); Peter S. Mezes, Old Lyme, CT (US); Charles E. Miller, Guilford, CT (US); Isabelle Millet, Milford, CT (US); Vishnu Mishra, Gainesville, FL (US); Muralidhara Padigaru, Branford, CT (US); Meera Patturajan, Branford, CT (US); Carol E. A. Pena, New Haven, CT (US); John A. Peyman, New Haven, CT (US); Luca Rastelli, Guilford, CT (US); Daniel K. Rieger, Branford, CT (US); Mark E. Rothenberg, Clinton, CT (US); Suresh G. Shenoy, Branford, CT (US); Richard A. Shimkets, Guilford, CT (US); Glennda Smithson, Guilford, CT (US); Steven K. Spaderna, Berlin, CT (US); Gary Starling, Clinton, CT (US); Kimberly A. Spytek, New Haven, CT (US); David J. Stone, Guilford, CT (US); Velizar T. Tchernev, Branford, CT (US); Nancy Twomlow, Madison, CT (US); Corine A.M. Vernet, Branford, CT (US); Bryan D. Zerhusen, Branford, CT (US); Edward Z. Voss, Wallingford, CT (US); Mei Zhong, Branford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/287,971

(22) Filed: Nov. 5, 2002

Related U.S. Application Data
(63) Continuation-in-part of application No. 09/997,425, filed on Nov. 29, 2001.
(63) Continuation-in-part of application No. 10/035,568, filed on Oct. 22, 2001.
(60) Provisional application No. 60/338,626, filed on Nov. 5, 2001.
(60) Provisional application No. 60/401,479, filed on Aug. 6, 2002.
(60) Provisional application No. 60/333,072, filed on Nov. 6, 2001.
(60) Provisional application No. 60/348,283, filed on Nov. 9, 2001.
(60) Provisional application No. 60/393,262, filed on Jul. 2, 2002.
(60) Provisional application No. 60/406,181, filed on Aug. 26, 2002.
(60) Provisional application No. 60/345,398, filed on Nov. 9, 2001.
(60) Provisional application No. 60/335,610, filed on Nov. 15, 2001.
(60) Provisional application No. 60/380,968, filed on May 15, 2002.
(60) Provisional application No. 60/332,152, filed on Nov. 21, 2001.
(60) Provisional application No. 60/336,576, filed on Dec. 4, 2001.
(60) Provisional application No. 60/354,807, filed on Feb. 5, 2002.
(60) Provisional application No. 60/393,148, filed on Jul. 2, 2002.
(60) Provisional application No. 60/401,626, filed on Aug. 6, 2002.
(60) Provisional application No. 60/401,695, filed on Aug. 7, 2002.
(60) Provisional application No. 60/333,912, filed on Nov. 28, 2001.
(60) Provisional application No. 60/381,043, filed on May 16, 2002.
(60) Provisional application No. 60/401,593, filed on Aug. 7, 2002.
(60) Provisional application No. 60/334,300, filed on Nov. 29, 2001.

(51) Int. Cl. G01N 33/53; A61K 38/17; C07K 14/47

(52) U.S. Cl. 514/12; 530/350; 435/7.1

(57) ABSTRACT

Disclosed herein are nucleic acid sequences that encode novel polypeptides. Also disclosed are polypeptides encoded by these nucleic acid sequences, and antibodies that immunospecifically bind to the polypeptide, as well as derivatives, variants, mutants, or fragments of the novel polypeptide, polynucleotide, or antibody specific to the polypeptide. Vectors, host cells, antibodies and recombinant methods for producing the polypeptides and polynucleotides, as well as methods for using same are also included. The invention further discloses therapeutic, diagnostic and research methods for diagnosis, treatment, and prevention of disorders involving any one of these novel human nucleic acids and proteins.

### RELATED APPLICATIONS

This application is a continuation-in-part of U.S. Ser. No. 09/997,425, filed Nov. 29, 2001 and U.S. Ser. No. 10/035,568, filed Oct. 22, 2001; this application also claims priority to provisional patent applications U.S. Ser. No. 60/338,626, filed Nov. 5, 2001, U.S. Ser. No. 60/401,479, filed Aug. 6, 2002, U.S. Ser. No. 60/333,072, filed Nov. 6, 2001, U.S. Ser. No. 60/348,283, filed Nov. 9, 2001, U.S. Ser. No. 60/393,262, filed Jul. 2, 2002, U.S. Ser. No. 60/406,181, filed Aug. 26, 2002, U.S. Ser. No. 60/345,398, filed Nov. 9, 2001, U.S. Ser. No. 60/335,610, filed Nov. 15, 2001, U.S. Ser. No. 60/380,968, filed May 15, 2002, U.S. Ser. No. 60/332,152, filed Nov. 21, 2001, U.S. Ser. No. 60/336,576, filed Dec. 4, 2001, U.S. Ser. No. 60/354,807, filed Feb. 5, 2002, U.S. Ser. No. 60/393,148, filed Jul. 2, 2002, U.S. Ser. No. 60/401,626, filed Aug. 6, 2002, U.S. Ser. No. 60/401,695, filed Aug. 7, 2002, U.S. Ser. No. 60/333,912, filed Nov. 28, 2001, U.S. Ser. No. 60/381,043, filed May 16, 2002, U.S. Ser. No. 60/401,593, filed Aug. 7, 2002, U.S. Ser. No. 60/334,300, filed Nov. 29, 2001, each of which is incorporated herein by reference in its entirety.

### FIELD OF THE INVENTION

The present invention relates to novel polypeptides, and the nucleic acids encoding them, having properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as methods of treating diverse pathological conditions.

### BACKGROUND OF THE INVENTION

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways involve extracellular signaling proteins, cellular receptors that bind the signaling proteins, and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including by way of nonlimiting example induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors. In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed levels of the protein effector of interest. In addition, there is a need for a method of treatment of a pathological condition brought on by a increased or up-regulated levels of the protein effector of interest.

Antibodies are multichain proteins that bind specifically to a given antigen, and bind poorly, or not at all, to substances deemed not to be cognate antigens. Antibodies are comprised of two short chains termed light chains and two long chains termed heavy chains. These chains are constituted of immunoglobulin domains, of which generally there are two classes: one variable domain per chain, one constant domain in light chains, and three or more constant domains in heavy chains. The antigen-specific portion of the immunoglobulin molecules resides in the variable domains; the variable domains of one light chain and one heavy chain associate with each other to generate the antigen-binding moiety. Antibodies that bind immunospecifically to a cognate or target antigen bind with high affinities. Accordingly, they are useful in assaying specifically for the presence of the antigen in a sample. In addition, they have the potential of inactivating the activity of the antigen.

Therefore there is a need to assay for the level of a protein effector of interest in a biological sample from such a subject, and to compare this level with that characteristic of a nonpathological condition. In particular, there is a need for such an assay based on the use of an antibody that binds immunospecifically to the antigen. There further is a need to inhibit the activity of the protein effector in cases where a pathological condition arises from elevated or excessive levels of the effector based on the use of an antibody that binds immunospecifically to the effector. Thus, there is a need for the antibody as a product of manufacture. There further is a need for a method of treatment of a pathological condition brought on by an elevated or excessive level of the protein effector of interest based on administering the antibody to the subject.

### SUMMARY OF THE INVENTION

The invention is based in part upon the discovery of isolated polypeptides including amino acid sequences selected from mature forms of the amino acid sequences selected from the group consisting of SEQ ID NO:2n, wherein n is an integer between 1 and 141. The novel nucleic acids and polypeptides are referred to herein as NOVX, or NOV1, NOV2, NOV3, etc., nucleic acids and polypeptides. These nucleic acids and polypeptides, as well as derivatives, homologs, analogs and fragments thereof, will hereinafter be collectively designated as “NOVX” nucleic acid or polypeptide sequences.

The invention also is based in part upon variants of a mature form of the amino acid sequence selected from the group consisting of SEQ ID NO:2n, wherein n is an integer between 1 and 141, wherein any amino acid in the mature form is changed to a different amino acid, provided that no more than 15% of the amino acid residues in the sequence of the mature form are so changed. In another embodiment, the invention includes the amino acid sequences selected from the group consisting of SEQ ID NO:2n, wherein n is an integer between 1 and 141. In another embodiment, the invention also comprises variants of the amino acid sequence selected from the group consisting of SEQ ID NO:2n, wherein n is an integer between 1 and 141 wherein any amino acid specified in the chosen sequence is changed to a different amino acid, provided that no more than 15% of the amino acid residues in the sequence are so changed. The invention also involves fragments of any of the mature forms of the amino acid sequences selected from the group consisting of SEQ ID NO:2n, wherein n is an integer between 1 and 141, or any other amino acid sequence selected from this group. The invention also comprises fragments from these groups in which up to 15% of the residues are changed.

In another embodiment, the invention encompasses polypeptides that are naturally occurring allelic variants of the sequence selected from the group consisting of SEQ ID NO:2n, wherein n is an integer between 1 and 141. These allelic variants include amino acid sequences that are the translations of nucleic acid sequences differing by a single nucleotide from nucleic acid sequences selected from the group consisting of SEQ ID NOS: 2n−1, wherein n is an integer between 1 and 141. The variant polypeptide where any amino acid changed in the chosen sequence is changed to provide a conservative substitution.

In another embodiment, the invention comprises a pharmaceutical composition involving a polypeptide with an amino acid sequence selected from the group consisting of SEQ ID NO:2n, wherein n is an integer between 1 and 141 and a pharmaceutically acceptable carrier. In another embodiment, the invention involves a kit, including, in one or more containers, this pharmaceutical composition.

In another embodiment, the invention includes the use of a therapeutic in the manufacture of a medicament for treating a syndrome associated with a human disease, the disease being selected from a pathology associated with a polypeptide with an amino acid sequence selected from the group consisting of SEQ ID NO:2n, wherein n is an integer between 1 and 141 wherein said therapeutic is the polypeptide selected from this group.

In another embodiment, the invention comprises a method for determining the presence or amount of a polypeptide with an amino acid sequence selected from the group consisting of SEQ ID NO:2n, wherein n is an integer between 1 and 141 in a sample, the method involving providing the sample; introducing the sample to an antibody that binds immunospecifically to the polypeptide; and determining the presence or amount of antibody bound to the polypeptide, thereby determining the presence or amount of polypeptide in the sample.

In another embodiment, the invention includes a method for determining the presence of or predisposition to a disease associated with altered levels of a polypeptide with an amino acid sequence selected from the group consisting of SEQ ID NO:2n, wherein n is an integer between 1 and 141 in a first mammalian subject, the method involving measuring the level of expression of the polypeptide in a sample from the first mammalian subject; and comparing the amount of the polypeptide in this sample to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In another embodiment, the invention involves a method of identifying an agent that binds to a polypeptide with an amino acid sequence selected from the group consisting of SEQ ID NO:2n, wherein n is an integer between 1 and 141, the method including introducing the polypeptide to the agent; and determining whether the agent binds to the polypeptide. The agent could be a cellular receptor or a downstream effector.

In another embodiment, the invention involves a method for identifying a potential therapeutic agent for use in treatment of a pathology, wherein the pathology is related to aberrant expression or aberrant physiological interactions of a polypeptide with an amino acid sequence selected from the group consisting of SEQ ID NO:2n, wherein n is an integer between 1 and 141, the method including providing a cell expressing the polypeptide of the invention and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent.

In another embodiment, the invention involves a method for screening for a modulator of activity or of latency or predisposition to a pathology associated with a polypeptide having an amino acid sequence selected from the group consisting of SEQ ID NO:2n, wherein n is an integer between 1 and 141, the method including administering a test compound to a test animal at increased risk for a pathology associated with the polypeptide of the invention, wherein the test animal recombinantly expresses the polypeptide of the invention; measuring the activity of the polypeptide in the test animal after administering the test compound; and comparing the activity of the protein in the test animal with the activity of the polypeptide in a control animal not administered the polypeptide, wherein a change in the activity of the polypeptide in the test animal relative to the control animal indicates the test compound is a modulator of latency of, or predisposition to, a pathology associated with the polypeptide of the invention. The recombinant test animal could express a test protein transgene or express the transgene under the control of a promoter at an increased level relative to a wild-type test animal The promoter may or may not b the native gene promoter of the transgene.

In another embodiment, the invention involves a method for modulating the activity of a polypeptide with an amino acid sequence selected from the group consisting of SEQ ID NO:2n, wherein n is an integer between 1 and 141, the method including introducing a cell sample expressing the polypeptide with a compound that binds to the polypeptide in an amount sufficient to modulate the activity of the polypeptide.

In another embodiment, the invention involves a method of treating or preventing a pathology associated with a polypeptide with an amino acid sequence selected from the group consisting of SEQ ID NO:2n, wherein n is an integer between 1 and 141, the method including administering the polypeptide to a subject in which such treatment or prevention is desired in an amount sufficient to treat or prevent the pathology in the subject. The subject could be human.

In another embodiment, the invention involves a method of treating a pathological state in a mammal, the method including administering to the mammal a polypeptide in an amount that is sufficient to alleviate the pathological state, wherein the polypeptide is a polypeptide having an amino acid sequence at least 95% identical to a polypeptide having the amino acid sequence selected from the group consisting of SEQ ID NO:2n, wherein n is an integer between 1 and 141 or a biologically active fragment thereof.

In another embodiment, the invention involves an isolated nucleic acid molecule comprising a nucleic acid sequence encoding a polypeptide having an amino acid sequence selected from the group consisting of a mature form of the amino acid sequence given SEQ ID NO:2n, wherein n is an integer between 1 and 141; a variant of a mature form of the amino acid sequence selected from the group consisting of SEQ ID NO:2n, wherein n is an integer between 1 and 141 wherein any amino acid in the mature form of the chosen sequence is changed to a different amino acid, provided that no more than 15% of the amino acid residues in the sequence of the mature form are so changed; the amino acid sequence selected from the group consisting of SEQ ID NO:2n, wherein n is an integer between 1 and 141; a variant of the amino acid sequence selected from the group consisting of SEQ ID NO:2n, wherein n is an integer between 1 and 141, in which any amino acid specified in the chosen sequence is changed to a different amino acid, provided that no more than 15% of the amino acid residues in the sequence are so changed; a nucleic acid fragment encoding at least a portion of a polypeptide comprising the amino acid sequence selected from the group consisting of SEQ ID NO:2n, wherein n is an integer between 1 and 141 or any variant of the polypeptide wherein any amino acid of the chosen sequence is changed to a different amino acid, provided that no more than 10% of the amino acid residues in the sequence are so changed; and the complement of any of the nucleic acid molecules.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO:2n, wherein n is an integer between 1 and 141, wherein the nucleic acid molecule comprises the nucleotide sequence of a naturally occurring allelic nucleic acid variant.

In another embodiment, the invention involves an isolated nucleic acid molecule including a nucleic acid sequence encoding a polypeptide having an amino acid sequence selected from the group consisting of a mature form of the amino acid sequence given SEQ ID NO:2n, wherein n is an integer between 1 and 141 that encodes a variant polypeptide, wherein the variant polypeptide has the polypeptide sequence of a naturally occurring polypeptide variant.

In another embodiment, the invention comprises an isolated nucleic, acid molecule having a nucleic acid sequence encoding a polypeptide comprising an amino acid sequence selected from the group consisting of a mature form of the amino acid sequence given SEQ ID NO:2n, wherein n is an integer between 1 and 141, wherein the nucleic acid molecule differs by a single nucleotide from a nucleic acid sequence selected from the group consisting of SEQ ID NOS: 2n−1, wherein n is an integer between 1 and 141.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 141, wherein the nucleic acid molecule comprises a nucleotide sequence selected from the group consisting of the nucleotide sequence selected from the group consisting of SEQ ID NO:2n−1, wherein n is an integer between 1 and 141; a nucleotide sequence wherein one or more nucleotides in the nucleotide sequence selected from the group consisting of SEQ ID NO:2n−1, wherein n is an integer between 1 and 141 is changed from that selected from the group consisting of the chosen sequence to a different nucleotide provided that no more than 15% of the nucleotides are so changed; a nucleic acid fragment of the sequence selected from the group consisting of SEQ ID NO:2n−1, wherein n is an integer between 1 and 141; and a nucleic acid fragment wherein one or more nucleotides in the nucleotide sequence selected from the group consisting of SEQ ID NO:2n−1, wherein n is an integer between 1 and 141 is changed from that selected from the group consisting of the chosen sequence to a different nucleotide provided that no more than 15% of the nucleotides are so changed.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 141, wherein the nucleic acid molecule hybridizes under stringent conditions to the nucleotide sequence selected from the group consisting of SEQ ID NO:2n−1, wherein n is an integer between 1 and 141, or a complement of the nucleotide sequence.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 141, wherein the nucleic acid molecule has a nucleotide sequence in which any nucleotide specified in the coding sequence of the chosen nucleotide sequence is changed from that selected from the group consisting of the chosen sequence to a different nucleotide provided that no more than 15% of the nucleotides in the chosen coding sequence are so changed, an isolated second polynucleotide that is a complement of the first polynucleotide, or a fragment of any of them.

In another embodiment, the invention includes a vector involving the nucleic acid molecule having a nucleic acid sequence encoding a polypeptide including an amino acid sequence selected from the group consisting of a mature form of the amino acid sequence given SEQ ID NO:2n, wherein n is an integer between 1 and 141. This vector can have a promoter operably linked to the nucleic acid molecule. This vector can be located within a cell.

In another embodiment, the invention involves a method for determining the presence or amount of a nucleic acid molecule having a nucleic acid sequence encoding a polypeptide including an amino acid sequence selected from the group consisting of a mature form of the amino acid sequence given SEQ ID NO:2n, wherein n is an integer between 1 and 141 in a sample, the method including providing the sample; introducing the sample to a probe that binds to the nucleic acid molecule; and determining the presence or amount of the probe bound to the nucleic acid molecule, thereby determining the presence or amount of the nucleic acid molecule in the sample. The presence or amount of the nucleic acid molecule is used as a marker for cell or tissue type. The cell type can be cancerous.

In another embodiment, the invention involves a method for determining the presence of or predisposition for a disease associated with altered levels of a nucleic acid molecule having a nucleic acid sequence encoding a polypeptide including an amino acid sequence selected from the group consisting of a mature form of the amino acid sequence given SEQ ID NO:2n, wherein n is an integer between 1 and 141 in a first mammalian subject, the method including measuring the amount of the nucleic acid in a sample from the first mammalian subject; and comparing the amount of the nucleic acid in the sample of step (a) to the amount of the nucleic acid present in a control sample from a second mammalian subject known not to have or not be predisposed to, the disease; wherein an alteration in the level of the nucleic acid in the first subject as compared to the control sample indicates the presence of or predisposition to the disease.

The invention further provides an antibody that binds immunospecifically to a NOVX polypeptide. The NOVX antibody may be monoclonal, humanized, or a fully human antibody. Preferably, the antibody has a dissociation constant for the binding of the NOVX polypeptide to the antibody less than 1×10⁻⁹ M. More preferably, the NOVX antibody neutralizes the activity of the NOVX polypeptide.

In a further aspect, the invention provides for the use of a therapeutic in the manufacture of a medicament for treating a syndrome associated with a human disease, associated with a NOVX polypeptide. Preferably the therapeutic is a NOVX antibody.

In yet a further aspect, the invention provides a method of treating or preventing a NOVX-associated disorder, a method of treating a pathological state in a mammal, and a method of treating or preventing a pathology associated with a polypeptide by administering a NOVX antibody to a subject in an amount sufficient to treat or prevent the disorder.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID NO | SEQ ID NO | |
|---|---|---|---|---|
| NOVX | Internal | (nucleic | (amino | |
| Assignment | Identification | acid) | acid) | Homology |
| 1a | CG103134-01 | 1 | 2 | Kunitz-type Protease |
| | | | | Inhibitor 2 |
| | | | | precursor-like |
| 1b | CG103134-02 | 3 | 4 | Kunitz-type Protease |
| | | | | Inhibitor 2 |
| | | | | precursor-like |
| 2a | CG103322-01 | 5 | 6 | CD82 Antigen-like |
| 2b | CG103322-02 | 7 | 8 | CD82 Antigen-like |
| 3a | CG151575-01 | 9 | 10 | Multi-pass Membrane |
| | | | | Protein-like |
| 3b | CG151575-02 | 11 | 12 | Multi-pass Membrane |
| | | | | Protein-like |
| 4a | CG151608-01 | 13 | 14 | Type 1b Membrane |
| | | | | Protein-like |
| 4b | CG151608-02 | 15 | 16 | Type 1b Membrane |
| | | | | Protein-like |
| 5a | CG152323-01 | 17 | 18 | Laminin beta 4-like |
| 6a | CG153011-01 | 19 | 20 | Sushi Domain-containing |
| | | | | Membrane Protein-like |
| 7a | CG153042-01 | 21 | 22 | RIK Protein-like |
| 7b | CG153042-02 | 23 | 24 | RIK Protein-like |
| 8a | CG153179-01 | 25 | 26 | Membrane Protein-like |
| 9a | CG153403-01 | 27 | 28 | Dickkopf Related |
| | | | | Protein-4 |
| | | | | Precursor-like |
| 9b | CG153403-02 | 29 | 30 | Dickkopf Related |
| | | | | Protein-4 |
| | | | | Precursor-like |
| 9c | 305037558 | 31 | 32 | Dickkopf Related |
| | | | | Protein-4 |
| | | | | Precursor-like |
| 9d | 305037512 | 33 | 34 | Dickkopf Related |
| | | | | Protein-4 |
| | | | | Precursor-like |
| 10a | CG153424-01 | 35 | 36 | IGFBP4-like |
| 11a | CG157567-01 | 37 | 38 | Leucine Rich Repeat |
| | | | | Protein-like |
| 12a | CG157760-01 | 39 | 40 | Placental Specific |
| | | | | Protein 1-like |
| 12b | CG157760-02 | 41 | 42 | Placental Specific |
| | | | | Protein 1-like |
| 13a | CG157844-01 | 43 | 44 | Type IIIb Membrane |
| | | | | Protein-like |
| 14a | CG158114-01 | 45 | 46 | Silver-like |
| 15a | CG158553-01 | 47 | 48 | Erythropoietin |
| | | | | Receptor-like |
| 15b | CG158553-01 | 49 | 50 | Erythropoietin |
| | | | | Receptor-like |
| 15c | CG158553-02 | 51 | 52 | Erythropoietin |
| | | | | Receptor-like |
| 15d | CG158553-03 | 53 | 54 | Erythropoietin |
| | | | | Receptor-like |
| 16a | CG158983-01 | 55 | 56 | Chloride Channel-like |
| 16b | CG158983-02 | 57 | 58 | Chloride Channel-like |
| 16c | CG158983-03 | 59 | 60 | Chloride Channel-like |
| 16d | CG158983-01 | 61 | 62 | Chloride Channel-like |
| 16e | CG158983-01 | 63 | 64 | Chloride Channel-like |
| 17a | CG159015-01 | 65 | 66 | Secreted Protein-like |
| 17b | CG159015-02 | 67 | 68 | Secreted Protein-like |
| 17c | CG159015-03 | 69 | 70 | Secreted Protein-like |
| 17d | CG159015-04 | 71 | 72 | Secreted Protein-like |
| 18a | CG173007-01 | 73 | 74 | Prolactin Receptor |
| | | | | Precursor-like |
| 19a | CG173357-01 | 75 | 76 | Immunoglobulin Domain |
| | | | | Containing Protein-like |
| 20a | CG50387-01 | 77 | 78 | Connexin 46 |
| 20b | CG50387-03 | 79 | 80 | Connexin 46 |
| 20c | CG50387-02 | 81 | 82 | Connexin 46 |
| 21a | CG52113-01 | 83 | 84 | Notch4-like |
| 21b | CG52113-06 | 85 | 86 | Notch4-like |
| 21c | 274054261 | 87 | 88 | Notch4-like |
| 21d | 274054299 | 89 | 90 | Notch4-like |
| 21e | 274054261 | 91 | 92 | Notch4-like |
| 21f | 274054299 | 93 | 94 | Notch4-like |
| 21g | CG52113-02 | 95 | 96 | Notch4-like |
| 21h | CG52113-03 | 97 | 98 | Notch4-like |
| 21i | CG52113-04 | 99 | 100 | Notch4-like |
| 21j | CG52113-05 | 101 | 102 | Notch4-like |
| 22a | CG57542-01 | 103 | 104 | Cadherin-23 |
| | | | | Precursor-like |
| 22b | 169258612 | 105 | 106 | Cadherin-23 |
| | | | | Precursor-like |
| 22c | 169258615 | 107 | 108 | Cadherin-23 |
| | | | | Precursor-like |
| 22d | 169258621 | 109 | 110 | Cadherin-23 |
| | | | | Precursor-like |
| 22e | 174307774 | 111 | 112 | Cadherin-23 |
| | | | | Precursor-like |
| 23a | CG57774-01 | 113 | 114 | TRNFR-19 Protein |
| 23b | 167200132 | 115 | 116 | TRNFR-19 Protein |
| 23c | 167200144 | 117 | 118 | TRNFR-19 Protein |
| 23d | 169252408 | 119 | 120 | TRNFR-19 Protein |
| 23e | 169252412 | 121 | 122 | TRNFR-19 Protein |
| 23f | 169252424 | 123 | 124 | TRNFR-19 Protein |
| 23g | 169252469 | 125 | 126 | TRNFR-19 Protein |
| 23h | 169252475 | 127 | 128 | TRNFR-19 Protein |
| 23i | 169252481 | 129 | 130 | TRNFR-19 Protein |
| 23j | 169252485 | 131 | 132 | TRNFR-19 Protein |
| 23k | 169252492 | 133 | 134 | TRNFR-19 Protein |
| 23l | 174104491 | 135 | 136 | TRNFR-19 Protein |
| 23m | 169252509 | 137 | 138 | TRNFR-19 Protein |
| 23n | 169252515 | 139 | 140 | TRNFR-19 Protein |
| 23o | 169252519 | 141 | 142 | TRNFR-19 Protein |
| 23p | 169252524 | 143 | 144 | TRNFR-19 Protein |
| 23q | 169252528 | 145 | 146 | TRNFR-19 Protein |
| 23r | 169252547 | 147 | 148 | TRNFR-19 Protein |
| 23s | 169252557 | 149 | 150 | TRNFR-19 Protein |
| 23t | 174104491 | 151 | 152 | TRNFR-19 Protein |
| 23u | CG57774-02 | 153 | 154 | TRNFR-19 Protein |
| 23v | CG57774-03 | 155 | 156 | TRNFR-19 Protein |
| 23w | CG57774-04 | 157 | 158 | TRNFR-19 Protein |
| 23x | CG57774-05 | 159 | 160 | TRNFR-19 Protein |
| 23y | CG57774-06 | 161 | 162 | TRNFR-19 Protein |
| 23z | CG57774-07 | 163 | 164 | TRNFR-19 Protein |
| 23aa | CG57774-08 | 165 | 166 | TRNFR-19 Protein |
| 23ab | CG57774-09 | 167 | 168 | TRNFR-19 Protein |
| 23ac | CG57774-10 | 169 | 170 | TRNFR-19 Protein |
| 23ad | CG57774-11 | 171 | 172 | TRNFR-19 Protein |
| 23ae | CG57774-12 | 173 | 174 | TRNFR-19 Protein |
| 23af | CG57774-13 | 175 | 176 | TRNFR-19 Protein |
| 24a | CG89285-01 | 177 | 178 | Alpha-1-Antichymotrypsin- |
| | | | | like |
| 24b | CG89285-04 | 179 | 180 | Alpha-1-Antichymotrypsin- |
| | | | | like |
| 24c | CG89285-03 | 181 | 182 | Alpha-1-Antichymotrypsin- |
| | | | | like |
| 24d | 306418132 | 183 | 184 | Alpha-1-Antichymotrypsin- |
| | | | | like |
| 24e | CG89285-02 | 185 | 186 | Alpha-1-Antichymotrypsin- |
| | | | | like |
| 25a | CG57094-01 | 187 | 188 | Human angiopoietin-like |
| 25b | 170075926 | 189 | 190 | Human angiopoietin-like |
| 25c | 164225601 | 191 | 192 | Human angiopoietin-like |
| 25d | 164225637 | 193 | 194 | Human angiopoietin-like |
| 25e | 170075926 | 195 | 196 | Human angiopoietin-like |
| 25f | 254120574 | 197 | 198 | Human angiopoietin-like |
| 25g | 254156650 | 199 | 200 | Human angiopoietin-like |
| 25h | 254500366 | 201 | 202 | Human angiopoietin-like |
| 25i | 226679956 | 203 | 204 | Human angiopoietin-like |
| 25j | 254500319 | 205 | 206 | Human angiopoietin-like |
| 25k | 254500445 | 207 | 208 | Human angiopoietin-like |
| 25l | 248210290 | 209 | 210 | Human angiopoietin-like |
| 25m | 252514148 | 211 | 212 | Human angiopoietin-like |
| 25n | 252514189 | 213 | 214 | Human angiopoietin-like |
| 25o | 252514198 | 215 | 216 | Human angiopoietin-like |
| 25p | 252514202 | 217 | 218 | Human angiopoietin-like |
| 25q | 228039766 | 219 | 220 | Human angiopoietin-like |
| 25r | 226679952 | 221 | 222 | Human angiopoietin-like |
| 25s | CG57094-02 | 223 | 224 | Human angiopoietin-like |
| 25t | CG57094-03 | 225 | 226 | Human angiopoietin-like |
| 25u | CG57094-04 | 227 | 228 | Human angiopoietin-like |
| 25v | CG57094-05 | 229 | 230 | Human angiopoietin-like |
| 25w | CG57094-06 | 231 | 232 | Human angiopoietin-like |
| 25x | CG57094-07 | 233 | 234 | Human angiopoietin-like |
| 25y | CG57094-08 | 235 | 236 | Human angiopoietin-like |
| 25z | CG57094-09 | 237 | 238 | Human angiopoietin-like |
| 25aa | CG57094-10 | 239 | 240 | Human angiopoietin-like |
| 25ab | CG57094-11 | 241 | 242 | Human angiopoietin-like |
| 25ac | CG57094-12 | 243 | 244 | Human angiopoietin-like |
| 25ad | CG57094-13 | 245 | 246 | Human angiopoietin-like |
| 26a | CG51523-05 | 247 | 248 | Endozepine Related |
| | | | | Protein |
| | | | | Precursor-like |
| 26b | CG51523-05_{—} | 249 | 250 | Endozepine Related |
| | 164786042 | | | Protein |
| | | | | Precursor-like |
| 26c | CG51523-05_{—} | 251 | 252 | Endozepine Related |
| | 164732479 | | | Protein |
| | | | | Precursor-like |
| 26d | CG51523-05_{—} | 253 | 254 | Endozepine Related |
| | 164732506 | | | Protein |
| | | | | Precursor-like |
| 26e | CG51523-05_{—} | 255 | 256 | Endozepine Related |
| | 164732693 | | | Protein |
| | | | | Precursor-like |
| 26f | CG51523-05_{—} | 257 | 258 | Endozepine Related |
| | 164732709 | | | Protein |
| | | | | Precursor-like |
| 26g | CG51523-05_{—} | 259 | 260 | Endozepine Related |
| | 164718189 | | | Protein |
| | | | | Precursor-like |
| 26h | CG51523-05_{—} | 261 | 262 | Endozepine Related |
| | 164718193 | | | Protein |
| | | | | Precursor-like |
| 26i | CG51523-05_{—} | 263 | 264 | Endozepine Related |
| | 164718197 | | | Protein |
| | | | | Precursor-like |
| 26j | CG51523-05_{—} | 265 | 266 | Endozepine Related |
| | 164718205 | | | Protein |
| | | | | Precursor-like |
| 26k | CG51523-05_{—} | 267 | 268 | Endozepine Related |
| | 164718209 | | | Protein |
| | | | | Precursor-like |
| 26l | CG51523-05_{—} | 269 | 270 | Endozepine Related |
| | 164718213 | | | Protein |
| | | | | Precursor-like |
| 26m | CG51523-05_{—} | 271 | 272 | Endozepine Related |
| | 166190452 | | | Protein |
| | | | | Precursor-like |
| 26n | CG51523-05_{—} | 273 | 274 | Endozepine Related |
| | 166190467 | | | Protein |
| | | | | Precursor-like |
| 26o | CG51523-05_{—} | 275 | 276 | Endozepine Related |
| | 166190475 | | | Protein |
| | | | | Precursor-like |
| 26p | CG51523-05_{—} | 277 | 278 | Endozepine Related |
| | 166190498 | | | Protein |
| | | | | Precursor-like |
| 26q | CG51523-05_{—} | 279 | 280 | Endozepine Related |
| | 166190460 | | | Protein |
| | | | | Precursor-like |
| 26r | CG51523-05_{—} | 281 | 282 | Endozepine Related |
| | 166190483 | | | Protein |
| | | | | Precursor-like |

Table A indicates the homology of NOVX polypeptides to known protein families. Thus, the nucleic acids and polypeptides, antibodies and related compounds according to the invention corresponding to a NOVX as identified in column 1 of Table A will be useful in therapeutic and diagnostic applications implicated in, for example, pathologies and disorders associated with the known protein families identified in column 5 of Table A.

Pathologies, diseases, disorders and condition and the like that are associated with NOVX sequences include, but are not limited to: e.,g. cardiomyopathy, atherosclerosis, hypertension, congenital heart defects, aortic stenosis atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity metabolic disturbances associated with obesity, transplantation, adrenoleukodystrophy, congenital adrenal hyperplasia, prostate cancer, diabetes, metabolic disorders, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, infectious disease, anorexia, cancer-associated cachexia, cancer, neurodegenerative disorders, Alzheimer's Disease, Parkinson's Disorder, immune disorders, hematopoietic disorders, and the various dyslipidemias,] the metabolic syndrome X and wasting disorders associated with chronic diseases and various cancers, as well as conditions such as transplantation, neuroprotection, fertility, or regeneration (in vitro and in vivo).]

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table A, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table A.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g. detection of a variety of cancers.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 141; (b) a variant of a mature form of the amino acid sequence selected from the group consisting of SEQ ID NO: 2n, wherein n is an integer between 1 and 141, wherein any amino acid in the mature form is changed to a different amino acid, provided that no more than 15% of the amino acid residues in the sequence of the mature form are so changed; (c) an amino acid sequence selected from the group consisting of SEQ ID NO: 2n, wherein n is an integer between 1 and 141; (d) a variant of the amino acid sequence selected from the group consisting of SEQ ID NO:2n, wherein n is an integer between 1 and 141 wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO: 2n, wherein n is an integer between 1 and 141; (b) a variant of a mature form of the amino acid sequence selected from the group consisting of SEQ ID NO: 2n, wherein n is an integer between 1 and 141 wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO: 2n, wherein n is an integer between 1 and 141; (d) a variant of the amino acid sequence selected from the group consisting of SEQ ID NO: 2n, wherein n is an integer between 1 and 141, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 141 or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n−1, wherein n is an integer between 1 and 141; (b) a nucleotide sequence wherein one or more nucleotides in the nucleotide sequence selected from the group consisting of SEQ ID NO: 2n−1, wherein n is an integer between 1 and 141 is changed from that selected from the group consisting of the chosen sequence to a different nucleotide provided that no more than 15% of the nucleotides are so changed; (c) a nucleic acid fragment of the sequence selected from the group consisting of SEQ ID NO: 2n−1, wherein n is an integer between 1 and 141; and (d) a nucleic acid fragment wherein one or more nucleotides in the nucleotide sequence selected from the group consisting of SEQ ID NO: 2n- 1, wherein n is an integer between 1 and 141 is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e.g., host cell) in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single-stranded or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NO:2n−1, wherein n is an integer between 1 and 141, or a complement of this nucleotide sequence, can be isolated using standard molecular biology techniques and the sequence information provided herein. Using all or a portion of the nucleic acid sequence of SEQ ID NO:2n−1, wherein n is an integer between 1 and 141, as a hybridization probe, NOVX molecules can be isolated using standard hybridization and cloning techniques (e.g., as described in Sambrook, et al., (eds.), MOLECULAR CLONING: A LABORATORY MANUAL 2nd Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO:2n−1, wherein n is an integer between 1 and 141, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO:2n−1, wherein n is an integer between 1 and 141, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NO:2n−1, wherein n is an integer between 1 and 141, is one that is sufficiently complementary to the nucleotide sequence of SEQ ID NO:2n−1, wherein n is an integer between 1 and 141, that it can hydrogen bond with few or no mismatches to the nucleotide sequence shown in SEQ ID NO:2n−1, wherein n is an integer between 1 and 141, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

A “fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g. they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms.

Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO:2n−1, wherein n is an integer between 1 and 141, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:2n−1, wherein n is an integer between 1 and 141; or an anti-sense strand nucleotide sequence of SEQ ID NO:2n−1, wherein n is an integer between 1 and 141; or of a naturally occurring mutant of SEQ ID NO:2n−1, wherein n is an integer between 1 and 141.

Probes based on the human NOVX nucleotide sequences can be used to detect transcripts or genomic sequences encoding the same or homologous proteins. In various embodiments, the probe has a detectable label attached, e.g. the label can be a radioisotope, a fluorescent compound, an enzyme, or an enzyme co-factor. Such probes can be used as a part of a diagnostic test kit for identifying cells or tissues which mis-express a NOVX. protein, such as by measuring a level of a NOVX-encoding nucleic acid in a sample of cells from a subject e.g., detecting NOVX mRNA levels or determining whether a genomic NOVX gene has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO:2n−1, wherein n is an integer between 1 and 141, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences of SEQ ID NO:2n−1, wherein n is an integer between 1 and 141, due to degeneracy of the genetic code and thus encode the same NOVX proteins as that encoded by the nucleotide sequences of SEQ ID NO:2n−1, wherein n is an integer between 1 and 141. In another embodiment, an isolated nucleic acid molecule of the invention has a nucleotide sequence encoding a protein having an amino acid sequence of SEQ ID NO:2n, wherein n is an integer between 1 and 141.

In addition to the human NOVX nucleotide sequences of SEQ ID NO:2n−1, wherein n is an integer between 1 and 141, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NO: 2n−1, wherein n is an integer between 1 and 141, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:2n−1, wherein n is an integer between 1 and 141. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6× SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2× SSC, 0.01% BSA at 50° C. An isolated nucleic acid molecule of the invention that hybridizes under stringent conditions to a sequence of SEQ ID NO:2n−1, wherein n is an integer between 1 and 141, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:2n−1, wherein n is an integer between 1 and 141, or fragments, analogs or derivatives thereof, under conditions of moderate stringency is provided. A non-limiting example of moderate stringency hybridization conditions are hybridization in 6× SSC, 5× Reinhardt's solution, 0.5% SDS and 100 mg/ml denatured salmon sperm DNA at 55° C., followed by one or more washes in 1× SSC, 0.1% SDS at 37° C. Other conditions of moderate stringency that may be used are well-known within the art. See, e.g., Ausubel, et aL (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NO:2n−1, wherein n is an integer between 1 and 141, or fragments, analogs or derivatives thereof, under conditions of low stringency, is provided. A non-limiting example of low stringency hybridization conditions are hybridization in 35% formamide, 5× SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2× SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NO:2n−1, wherein n is an integer between 1 and 141, thereby leading to changes in the amino acid sequences of the encoded NOVX protein, without altering the functional ability of that NOVX protein. For example, nucleotide substitutions leading to amino acid substitutions at “non-essential” amino acid residues can be made in the sequence of SEQ ID NO:2n, wherein n is an integer between 1 and 141. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO:2n−1, wherein n is an integer between 1 and 141, yet retain biological activity. In one embodiment, the isolated nucleic acid molecule comprises a nucleotide sequence encoding a protein, wherein the protein comprises an amino acid sequence at least about 40% homologous to the amino acid sequences of SEQ ID NO:2n, wherein n is an integer between 1 and 141. Preferably, the protein encoded by the nucleic acid molecule is at least about 60% homologous to SEQ ID NO:2n, wherein n is an integer between 1 and 141; more preferably at least about 70% homologous to SEQ ID NO:2n, wherein n is an integer between 1 and 141; still more preferably at least about 80% homologous to SEQ ID NO:2n, wherein n is an integer between 1 and 141; even more preferably at least about 90% homologous to SEQ ID NO:2n, wherein n is an integer between 1 and 141; and most preferably at least about 95% homologous to SEQ ID NO:2n, wherein n is an integer between 1 and 141.

An isolated nucleic acid molecule encoding a NOVX protein homologous to the protein of SEQ ID NO:2n, wherein n is an integer between 1 and 141, can be created by introducing one or more nucleotide substitutions, additions or deletions into the nucleotide sequence of SEQ ID NO:2n−1, wherein n is an integer between 1 and 141, such that one or more amino acid substitutions, additions or deletions are introduced into the encoded protein.

Mutations can be introduced any one of SEQ ID NO:2n−1, wherein n is an integer between 1 and 141, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NO:2n−1, wherein n is an integer between 1 and 141, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Interfering RNA

In one aspect of the invention, NOVX gene expression can be attenuated by RNA interference. One approach well-known in the art is short interfering RNA (siRNA) mediated gene silencing where expression products of a NOVX gene are targeted by specific double stranded NOVX derived siRNA nucleotide sequences that are complementary to at least a 19-25 nt long segment of the NOVX gene transcript, including the 5′ untranslated (UT) region, the ORF, or the 3′ UT region. See, e.g., PCT applications WO00/44895, WO99/32619, WO01/75164, WO01/92513, WO01/29058, WO01/89304, WO02/16620, and WO02/29858, each incorporated by reference herein in their entirety. Targeted genes can be a NOVX gene, or an upstream or downstream modulator of the NOVX gene. Nonlimiting examples of upstream or downstream modulators of a NOVX gene include, e.g., a transcription factor that binds the NOVX gene promoter, a kinase or phosphatase that interacts with a NOVX polypeptide, and polypeptides involved in a NOVX regulatory pathway.

According to the methods of the present invention, NOVX gene expression is silenced using short interfering RNA. A NOVX polynucleotide according to the invention includes a siRNA polynucleotide. Such a NOVX siRNA can be obtained using a NOVX polynucleotide sequence, for example, by processing the NOVX ribopolynucleotide sequence in a cell-free system, such as but not limited to a Drosophila extract, or by transcription of recombinant double stranded NOVX RNA or by chemical synthesis of nucleotide sequences homologous to a NOVX sequence. See, e.g., Tuschl, Zamore, Lehmann, Bartel and Sharp (1999), Genes & Dev. 13: 3191-3197, incorporated herein by reference in its entirety. When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxyribonucleotides in the 3′ overhangs is as efficient as using ribonucleotides, but deoxyribonucleotides are often cheaper to synthesize and are most likely more nuclease resistant.

A contemplated recombinant expression vector of the invention comprises a NOVX DNA molecule cloned into an expression vector comprising operatively-linked regulatory sequences flanking the NOVX sequence in a manner that allows for expression (by transcription of the DNA molecule) of both strands. An RNA molecule that is antisense to NOVX mRNA is transcribed by a first promoter (e.g., a promoter sequence 3′ of the cloned DNA) and an RNA molecule that is the sense strand for the NOVX mRNA is transcribed by a second promoter (e.g., a promoter sequence 5′ of the cloned DNA). The sense and antisense strands may hybridize in vivo to generate siRNA constructs for silencing of the NOVX gene. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is a siRNA. The regulatory sequences flanking the NOVX sequence may be identical or may be different, such that their expression may be modulated independently, or in a temporal or spatial manner.

In a specific embodiment, siRNAs are transcribed intracellularly by cloning the NOVX gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

A siRNA vector appears to have an advantage over synthetic siRNAs where long term knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A NOVX mRNA region to be targeted by siRNA is generally selected from a desired NOVX sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See, Elbashir et al. 2001 EMBO J. 20(23):6877-88. Hence, consideration should be taken to accommodate SNPs, polymorphisms, allelic variants or species-specific variations when targeting a desired gene.

In one embodiment, a complete NOVX siRNA experiment includes the proper negative control. A negative control siRNA generally has the same nucleotide composition as the NOVX siRNA but lack significant sequence homology to the genome. Typically, one would scramble the nucleotide sequence of the NOVX siRNA and do a homology search to make sure it lacks homology to any other gene.

Two independent NOVX siRNA duplexes can be used to knock-down a target NOVX gene. This helps to control for specificity of the silencing effect. In addition, expression of two independent genes can be simultaneously knocked down by using equal concentrations of different NOVX siRNA duplexes, e.g., a NOVX siRNA and an siRNA for a regulator of a NOVX gene or polypeptide. Availability of siRNA-associating proteins is believed to be more limiting than target mRNA accessibility.

A targeted NOVX region is typically a sequence of two adenines (AA) and two thymidines (TT) divided by a spacer region of nineteen (N19) residues (e.g., AA(N19)TT). A desirable spacer region has a G/C-content of approximately 30% to 70%, and more preferably of about 50%. If the sequence AA(N19)TT is not present in the target sequence, an alternative target region would be AA(N21). The sequence of the NOVX sense siRNA corresponds to (N19)TT or N21, respectively. In the latter case, conversion of the 3′ end of the sense siRNA to TT can be performed if such a sequence does not naturally occur in the NOVX polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs. See, e.g., Elbashir, Lendeckel and Tuschl (2001). Genes & Dev. 15: 188-200, incorporated by reference herein in its entirely. The modification of the overhang of the sense sequence of the siRNA duplex is not expected to affect targeted mRNA recognition, as the antisense siRNA strand guides target recognition.

Alternatively, if the NOVX target mRNA does not contain a suitable AA(N21) sequence, one may search for the sequence NA(N21). Further, the sequence of the sense strand and antisense strand may still be synthesized as 5′ (N19)TT, as it is believed that the sequence of the 3′-most nucleotide of the antisense siRNA does not contribute to specificity. Unlike antisense or ribozyme technology, the secondary structure of the target mRNA does not appear to have a strong effect on silencing. See, Harborth, et al. (2001) J. Cell Science 114: 4557-4565, incorporated by reference in its entirety.

Transfection of NOVX siRNA duplexes can be achieved using standard nucleic acid transfection methods, for example, OLIGOFECTAMINE Reagent (commercially available from Invitrogen). An assay for NOVX gene silencing is generally performed approximately 2 days after transfection. No NOVX gene silencing has been observed in the absence of transfection reagent, allowing for a comparative analysis of the wild-type and silenced NOVX phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g. inversion versus vortexing) are also critical. Low transfection efficiencies are the most frequent cause of unsuccessful NOVX silencing. The efficiency of transfection needs to be carefully examined for each new cell line to be used. Preferred cell are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense NOVX siRNA will have no effect on NOVX silencing, and 0.84 μg antisense siRNA has a weak silencing effect when compared to 0.84 μg of duplex siRNAs. Control experiments again allow for a comparative analysis of the wild-type and silenced NOVX phenotypes. To control for transfection efficiency, targeting of common proteins is typically performed, for example targeting of lamin A/C or transfection of a CMV-driven EGFP-expression plasmid (e.g. commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted NOVX polynucleotide in a cell, a knock-down phenotype may become apparent after 1 to 3 days, or even later. In cases where no NOVX knock-down phenotype is observed, depletion of the NOVX polynucleotide may be observed by immunofluorescence or Western blotting. If the NOVX polynucleotide is still abundant after 3 days, cells need to be split and transferred to a fresh 24-well plate for re-transfection. If no knock-down of the targeted protein is observed, it may be desirable to analyze whether the target mRNA (NOVX or a NOVX upstream or downstream gene) was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable NOVX protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering a NOVX siRNA construct as therapy to compensate for increased or aberrant NOVX expression or activity. The NOVX ribopolynucleotide is obtained and processed into siRNA fragments, or a NOVX siRNA is synthesized, as described above. The NOVX siRNA is administered to cells or tissues using known nucleic acid transfection techniques, as described above. A NOVX siRNA specific for a NOVX gene will decrease or knockdown NOVX transcription products, which will lead to reduced NOVX polypeptide production, resulting in reduced NOVX polypeptide activity in the cells or tissues.

The present invention also encompasses a method of treating a disease or condition associated with the presence of a NOVX protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the NOVX gene function is not correlated with a known phenotype, a control sample of cells or tissues from healthy individuals provides a reference standard for determining NOVX expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The NOVX ribopolynucleotide is used to produce siRNA constructs, that are specific for the NOVX gene product. These cells or tissues are treated by administering NOVX siRNA's to the cells or tissues by methods described for the transfection of nucleic acids into a cell or tissue, and a change in NOVX polypeptide or polynucleotide expression is observed in the subject sample relative to the control sample, using the assays described. This NOVX gene knockdown approach provides a rapid method for determination of a NOVX minus (NOVX⁻) phenotype in the treated subject sample. The NOVX⁻ phenotype observed in the treated subject sample thus serves as a marker for monitoring the course of a disease state during treatment.

In specific embodiments, a NOVX siRNA is used in therapy. Methods for the generation and use of a NOVX siRNA are known to those skilled in the art. Example techniques are provided below.

Production of RNAs

Sense RNA (ssRNA) and antisense RNA (asRNA) of NOVX are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al., Molecular Cloning. Cold Spring Harbor Laboratory Press, Plainview, N.Y. (1989).

Lysate Preparation

Untreated rabbit reticulocyte lysate (Ambion) are assembled according to the manufacturer's directions. dsRNA is incubated in the lysate at 30° C. for 10 min prior to the addition of mRNAs. Then NOVX mRNAs are added and the incubation continued for an additional 60 min. The molar ratio of double stranded RNA and mRNA is about 200:1. The NOVX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2× proteinase K buffer and deproteinized as described previously (Tuschl et al., Genes Dev., 13:3191-3197 (1999)). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing NOVX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation

21 nt RNAs, based on the sequence determined above, are chemically synthesized using Expedite RNA phosphoramidites and thymidine phosphoramidite (Proligo, Germany). Synthetic oligonucleotides are deprotected and gel-purified (Elbashir, Lendeckel, & Tuschl, Genes & Dev. 15, 188-200 (2001)), followed by Sep-Pak C18 cartridge (Waters, Milford, Mass., USA) purification (Tuschl, et al., Biochemistry, 32:11658-11668 (1993)).

These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37° C.

Cell Culture

A cell culture known in the art to regularly express NOVX is propagated using standard conditions. 24 hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit and NOVX expression is monitored using standard techniques with positive and negative control. A positive control is cells that naturally express NOVX while a negative control is cells that do not express NOVX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of NOVX siRNA sequence and the use of such siRNA for in vitro suppression. In vivo suppression may be performed using the same siRNA using well known in vivo transfection or gene therapy transfection techniques.

Antisense Nucleic Acids

Another aspect of the invention pertains to isolated antisense nucleic acid molecules that are hybridizable to or complementary to the nucleic acid molecule comprising the nucleotide sequence of SEQ ID NO:2n−1, wherein n is an integer between 1 and 141, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO:2n, wherein n is an integer between 1 and 141, or antisense nucleic acids complementary to a NOVX nucleic acid sequence of SEQ ID NO:2n−1, wherein n is an integer between 1 and 141, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl) uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 5-methoxyuracil, 3-methylcytosine, 5-methylcytosine, N6-adenine, 7-methylguanine, 5-methylaminomethyluracil, 5-methoxyaminomethyl-2-thiouracil, 2-thiouracil, 4-thiouracil, beta-D-mannosylqueosine, 5′-methoxycarboxymethyluracil, 2-methylthio-N6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an (α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., SEQ ID NO:2n−1, wherein n is an integer between 1 and 141). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NO:2n, wherein n is an integer between 1 and 141. The invention also includes a mutant or variant protein any of whose residues may be changed from the corresponding residues shown in any one of SEQ ID NO:2n, wherein n is an integer between 1 and 141, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO:2n, wherein n is an integer between 1 and 141) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO:2n, wherein n is an integer between 1 and 141. In other embodiments, the NOVX protein is substantially homologous to SEQ ID NO:2n, wherein n is an integer between 1 and 141, and retains the functional activity of the protein of SEQ ID NO:2n, wherein n is an integer between 1 and 141, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO:2n, wherein n is an integer between 1 and 141, and retains the functional activity of the NOVX proteins of SEQ ID NO:2n, wherein n is an integer between 1 and 141.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO:2n−1, wherein n is an integer between 1 and 141.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO:2n, wherein n is an integer between 1 and 141, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with SI nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO:2n, wherein n is an integer between 1 and 141, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is ≦1 μM, preferably ≦100 nM, more preferably ≦10 nM, and most preferably ≦100 pM to about 1 pM, as measured by assays such as radioligand binding assays or similar assays known to those skilled in the art.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No.5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fe), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO96/33735 and WO96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispeciric Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

In one embodiment, methods for the screening of antibodies that possess the desired specificity include, but are not limited to, enzyme linked immunosorbent assay (ELISA) and other immunologically mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Antibodies directed against a NOVX protein of the invention may be used in methods known within the art relating to the localization and/or quantitation of a NOVX protein (e.g., for use in measuring levels of the NOVX protein within appropriate physiological samples, for use in diagnostic methods, for use in imaging the protein, and the like). In a given embodiment, antibodies specific to a NOVX protein, or derivative, fragment, analog or homolog thereof, that contain the antibody derived antigen binding domain, are utilized as pharmacologically active compounds (referred to hereinafter as “Therapeutics”).

An antibody specific for a NOVX protein of the invention (e.g., a monoclonal antibody or a polyclonal antibody) can be used to isolate a NOVX polypeptide by standard techniques, such as immunoaffinity, chromatography or immunoprecipitation. An antibody to a NOVX polypeptide can facilitate the purification of a natural NOVX antigen from cells, or of a recombinantly produced NOVX antigen expressed in host cells. Moreover, such an anti-NOVX antibody can be used to detect the antigenic NOVX protein (e.g., in a cellular lysate or cell supernatant) in order to evaluate the abundance and pattern of expression of the antigenic NOVX protein. Antibodies directed against a NOVX protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and acquorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Thory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec 1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J 8: 729-733) and immunoglobulins (Banedji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infecfion) and allowing the oocyte to develop in a pseudopregnant female foster animal. The human NOVX cDNA sequences, i.e., any one of SEQ ID NO:2n−1, wherein n is an integer between 1 and 141, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NO:2n−1, wherein n is an integer between 1 and 141), but more preferably, is a non-human homologue of a human NOVX gene. For example, a mouse homologue of human NOVX gene of SEQ ID NO:2n−1, wherein n is an integer between 1 and 141, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. A NOVX target molecule can be a non-NOVX molecule or a NOVX protein or polypeptide of the invention. In one embodiment, a NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e. intracellular Ca²⁺ diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been-isolated, this sequence can be used to map the location of the gene on a chromosome. This process is called chromosome mapping. Accordingly, portions or fragments of the NOVX sequences of SEQ ID NO:2n−1, wherein n is an integer between 1 and 141, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NO:2n−1, wherein n is an integer between 1 and 141, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO:2n−1, wherein n is an integer between 1 and 141, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identifpy subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders. The disorders include but are not limited to, e.g., those diseases, disorders and conditions listed above, and more particularly include those diseases, disorders, or conditions associated with homologs of a NOVX protein, such as those summarized in Table A.

In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome pregnancy zone protein precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include but are not limited to, e.g., those diseases, disorders and conditions listed above, and more particularly include those diseases, disorders, or conditions associated with homologs of a NOVX protein, such as those summarized in Table A.

These methods of treatment will be discussed more fully, below.

Diseases and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders. The disorders include but are not limited to, e.g., those diseases, disorders and conditions listed above, and more particularly include those diseases, disorders, or conditions associated with homologs of a NOVX protein, such as those summarized in Table A.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from diseases, disorders, conditions and the like, including but not limited to those listed herein.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example A

### Polynucleotide and Polypeptide Sequences, and Homology Data

### Example 1

The NOV1 clone was analyzed, and the nucleotide and encoded polypeptide sequences are shown in Table 1A.

TABLE 1A
NOV1 Sequence Analysis

| | |
|---|---|
| SEQ ID NO: 1 | 968 bp |

| | |
|---|---|
| NOV1a, | ATGGGTCTGGCCATGGAGCAGCTGTCCGGGCTGAGGCGGAGCCGGGCGTTTCTCGCCC |
| CG103134-01 DNA | |
| Sequence | TGCTGGGATCGCTGCTCCTCTCTGGGGTCCTGGCGGCCGACCGAGAACGCAGCATCCA |
| | CGACTTCTGCCTGGTGTCGAAGGTGGTGGGCAGATGCCGGGCCTCCATGCCTAGGTGG |
| | TGGTACAATGTCACTGACGGATCCTGCCAGCTGTTTGTGTATGGGGGCTGTGACGGAA |
| | ACAGCAATAATTACCTGACCAAGGAGGAGTGCCTCAAGAAATGTGCCACTGTCACAGA |
| | GAATGCCACGGGTGACCTGGCCACCAGCAGGAATGCAGCGGATTCCTCTGTCCCAAGT |
| | GCTCCCAGAAGGCAGGATTCTGAAGACCACTCCAGCGATATGTTCAACTATGAAGAAT |
| | ACTGCACCGCCAACGCAGTCACTGGGCCTTGCCGTGCATCCTTCCCACCCTGGTACTT |
| | TGACGTGGAGAGGAACTCCTGCAATAACTTCATCTATGGAGGCTGCCGGGGCAATAAG |
| | AACAGCTACCGCTCTGAGGAGGCCTGCATGCTCCGCTGCTTCCGCCAGCAGGAGAATC |
| | CTCCCCTGCCCCTTGGCTCAAAGGTGGTGGTTCTGGCGGGGCTGTTCGTGATGGTGTT |
| | GATCCTCTTCCTGGGAGCCTCCATGGTCTACCTGATCCGGGTGGCACGGAGGAGCCAG |
| | GAGCGTGCCCTGCGCACCGTCTGGAGCTCCGGAGATGACAAGGAGCAGCTGGTGAAGA |
| | ACACATATGTCCTGTGACCGCCCTGTCGCCAAGAGGACTGGGGAAGGGAGGGGAGACT |
| | ATGTGTGAGCTTTTTTTAAATAGAGGGATTGACTCGGATTTGAGTGATCATTAGGGCT |
| | GAGGTCTGTTTCTCTGGGAGCTAGGACGGCTGCTTCCTGGTCTGGCAGGCATGGGTTT |
| | GCTTTGGAAATCCTCTACGAGGCTCCGGCACTGACCTAAG |

| | |
|---|---|
| ORF Start: ATG at 1 | ORF Stop: TGA at 769 |

| | | |
|---|---|---|
| SEQ ID NO: 2 | 256 aa | MW at 28631.3 kD |

| | |
|---|---|
| NOV1a, | MGLAMEQLCGLRRSRAFLALLGSLLLSGVLAADRERSIHDFCLVSKVVGRCRASMPRW |
| CG103134-01 | |
| Protein Sequence | WYNVTDGSCQLFVYGGCDGNSNNYLTKEECLKKCATVTENATCDLATSRNAADSSVPS |
| | APRRQDSEDHSSDMFNYEEYCTANAVTGPCRASFPRWYFDVERNSCNNFIYGCCRGNK |
| | NSYRSEEACMLRCFRQQENPPLPLGSKVVVLAGLFVMVLILFLGASMVYLIRVARRSQ |
| | ERALRTVWSSGDDKEQLVKNTYVL |

| | |
|---|---|
| SEQ ID NO: 3 | 869 bp |

| | |
|---|---|
| NOV1b, | GAGACCCCAACGGCTGGTGGCGTCGCCTGCGCGTCTCGGCTGAGCTGGCCATGGCGCA |
| CG103134-02 DNA | |
| Sequence | GCTGTGCGGGCTGAGGCGGAGCCGGGCGTTTCTCGCCCTGCTCGGATCGCTGCTCCTC |
| | TCTGGGGTCCTGGCGGCCGACCGAGAACGCAGCATCCACGGTGAGGGCCGGGCGGACT |
| | TCTGCCTGGTGTCGAAGGTGGTGGGCAGATGCCGGGCCTCCATGCCTAGGTGGTGGCA |
| | CAATGTCACTGACGGATCCTGCCAGCTGTTTGTGTATGGGGGCTGTGACGGAAACAGC |
| | AATAATTACCTGACCAAGGAGGAGTGCCTCAAGAAATGTGCCACTGTCACACAGAATG |
| | CCACGGGTGACCTGGCCACCAGCAGGAATGCAGCGGATTCCTCTGTCCCAAGTGCTCC |
| | CAGAACGCAGGATTCTGAAGACCACTCCAGCGATATGTTCAACTATGAAGAATACTGC |
| | ACCGCCAACGCAGTCACTGCGCCTTGCCGTGCATCCTTCCCACGCTGGTACTTTGACG |
| | TGGAGAGGAACTCCTGCAATAACTTCATCTATGGAGGCTGCCGGGGCAATAAGAACAG |
| | CTACCGCTCTGAGGAGGCCTGCATGCTCCCCTGCTTCCGCCAGCAGGAGAATCCTCCC |
| | CTGCCCCTTGGCTCAAAGGTGGTGGTTCTGGCGGGGCTGTTCGTGATGGTGTTGATCC |
| | TCTTCCTGGGAGCCTCCATGGTCTACCTGATCCGGGTGGCACGGAGGAACCAGGACCG |
| | TGCCCTGCGCACCGTCTGGAGCTCCGGAGATGACAAGGAGCAGCTGGTGAAGAACACA |
| | TATGTCCTGTGACCGGCCTGTCGCCAAGAGGACTGGGGAAGGGAGGGGAGACTATGG |

| | |
|---|---|
| ORF Start: ATG at 51 | ORF Stop: TGA at 822 |

| | | |
|---|---|---|
| SEQ ID NO: 4 | 257 aa | MW at 28672.2 kD |

| | |
|---|---|
| NOV1b, | MAQLCGLRRSRAFLALLGSLLLSGVLAADRERSIHGEGRADFCLVSKVVGRCRASMPR |
| CG103134-02 | |
| Protein Sequence | WWHNVTDGSCQLFVYGGCDGNSNNYLTKEECLKKCATVTENATGDLATSRNAADSSVP |
| | SAPRRQDSEDHSSDMFNYEEYCTANAVTGPCRASFPRWYFDVERNSCNNFIYGGCRGN |
| | KNSYRSEEACMLRCFRQQENPPLPLGSKVVVLAGLFVMVLILFLGASMVYLIRVARRN |
| | QERALRTVWSSGDDKEQLVKNTYVL |

Sequence comparison of the above protein sequences yields the following sequence relationships shown in Table 1B.

TABLE 1B
Comparison of NOV1a against NOV1b.

| | | Identities/ |
|---|---|---|
| | NOV1a Residues/ | Similarities for |
| Protein Sequence | Match Residues | the Matched Region |
| NOV1b | 5 . . . 256 | 249/257 (96%) |
| | 1 . . . 257 | 251/257 (96%) |

Further analysis of the NOV1a protein yielded the following properties shown in Table 1C.

TABLE 1C
Protein Sequence Properties NOV1a

| | |
|---|---|
| PSort | 0.8705 probability located in mitochondrial |
| analysis: | inner membrane; 0.6000 probability located |
| | in plasma membrane; 0.4983 probability located |
| | in mitochondrial intermembrane space; |
| | 0.4000 probability located in Golgi body |
| SignalP | Cleavage site between residues 32 and 33 |
| analysis: | |

A search of the NOV1a protein against the Geneseq database, a proprietary database that contains sequences published in patents and patent publication, yielded several homologous proteins shown in Table 1D.

TABLE 1D
Geneseq Results for NOV1a

| | | NOV1a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| ABP41951 | Human ovarian antigen | 3 . . . 256 | 252/254 (99%) | e−148 |
| | HDABR73, SEQ ID NO: 3083 - | 17 . . . 270 | 253/254 (99%) | |
| | Homo sapiens, 270 aa. | | | |
| | [WO200200677-A1, 03 JAN. 2002] | | | |
| AAB43821 | Human cancer associated | 3 . . . 256 | 252/254 (99%) | e−148 |
| | protein sequence SEQ ID | 17 . . . 270 | 253/254 (99%) | |
| | NO: 1266 - Homo sapiens, 289 | | | |
| | aa. [WO200055350-A1, 21 SEP. | | | |
| | 2000] | | | |
| AAO17719 | Human kunitz type protease | 5 . . . 256 | 250/252 (99%) | e−148 |
| | inhibitor bikunin - Homo | 1 . . . 252 | 251/252 (99%) | |
| | sapiens, 252 aa. [WO9957274- | | | |
| | A1, 11 NOV. 1999] | | | |
| AAB14187 | Human placental bikunin | 5 . . . 256 | 250/252 (99%) | e−148 |
| | protein # 5 - Homo sapiens, | 1 . . . 252 | 251/252 (99%) | |
| | 252 aa. [WO200037099-A2, 29 | | | |
| | JUN. 2000] | | | |
| AAW70286 | Human tissue factor pathway | 5 . . . 256 | 250/252 (99%) | e−148 |
| | inhibitor-3 (TFPI-3) - Homo | 1 . . . 252 | 251/252 (99%) | |
| | sapiens, 252 aa. [WO9833920- | | | |
| | A2, 06 AUG. 1998] | | | |

In a BLAST search of public sequence datbases, the NOV1a protein was found to have homology to the proteins shown in the BLASTP data in Table 1E.

TABLE 1E
Public BLASTP Results for NOV1a

| | | | Identities/ | |
|---|---|---|---|---|
| | | NOV1a | Similarities | |
| Protein | | Residues/ | for the | |
| Accession | | Match | Matched | Expect |
| Number | Protein/Organism/Length | Residues | Portion | Value |
| O43291 | Kunitz-type protease inhibitor | 5 . . . 256 | 250/252 (99%) | e−147 |
| | 2 precursor (Hepatocyte growth | 1 . . . 252 | 251/252 (99%) | |
| | factor activator inhibitor | | | |
| | type 2) (HAI-2) (Placental | | | |
| | bikunin) - Homo sapiens | | | |
| | (Human), 252 aa. | | | |
| Q9WU03 | Kunitz-type protease inhibitor | 5 . . . 256 | 177/252 (70%) | e−102 |
| | 2 precursor (Hepatocyte growth | 1 . . . 252 | 202/252 (79%) | |
| | factor activator inhibitor | | | |
| | type 2) (HAI-2) - Mus musculus | | | |
| | (Mouse), 252 aa. | | | |
| JG0185 | hepatocyte growth factor | 5 . . . 256 | 177/252 (70%) | e−102 |
| | activator inhibitor type 2 - | 1 . . . 252 | 201/252 (79%) | |
| | mouse, 252 aa. | | | |
| AAH03431 | Serine protease inhibitor, | 95 . . . 256 | 112/162 (69%) | 3e−60 |
| | Kunitz type 2 - Mus musculus | 34 . . . 195 | 129/162 (79%) | |
| | (Mouse), 195 aa. | | | |
| Q9D8Q8 | Serine protease inhibitor, | 95 . . . 256 | 112/162 (69%) | 3e−60 |
| | kunitz type 2 - Mus musculus | 34 . . . 195 | 129/162 (79%) | |
| | (Mouse), 195 aa. | | | |

PFam analysis predicts that the NOV1a protein contains the domains shown in the Table 1F.

TABLE 1F
Domain Analysis of NOV1a

| | | Identities/ | |
|---|---|---|---|
| | NOV1a Match | Similarities for | |
| Pfam Domain | Region | the Matched Region | Expect Value |
| Kunitz_BPTI | 42 . . . 92 | 24/62 (39%) | 9.7e−28 |
| | | 45/62 (73%) | |
| Kunitz_BPTI | 137 . . . 187 | 22/62 (35%) | 2.6e−22 |
| | | 39/62 (63%) | |

### Example 2

The NOV2 clone was analyzed, and the nucleotide and encoded polypeptide sequences are shown in Table 2A.

TABLE 2A
NOV2 Sequence Analysis

| | |
|---|---|
| SEQ ID NO: 5 | 841 bp |

| | |
|---|---|
| NOV2a, | ACTGGTTTCGTGGAAGGAAGCTCCAGGACTGGCGGGATGGGCTCAGCCTGTATCAAAG |
| CG103322-01 DNA | |
| Sequence | TCACCAAATACTTTCTCTTCCTCTTCAACTTGATCTTCTTTATCCTGGGCGCAGTGAT |
| | CCTGGGCTTCGGGGTGTGGATCCTGGCCGACAAGAGCAGTTTCATCTCTGTCCTGCAA |
| | ACCTCCTCCAGCTCGCTTAGGATGGGGGCCTATGTCTTCATCGGCGTGGGGGCAGTCA |
| | CTATGCTCATGGGCTTCCTGGGCTGCATCGGCGCCGTCAACGAGGTCCGCTGCCTGCT |
| | GGGGCTGTACTTTGCTTTCCTGCTCCTGATCCTCATTGCCCACGTGACGGCCGGGGCC |
| | CTCTTCTACTTCAACATGGGCAAGCTGAAGCAGGAGATGGGCCGCATCGTGACTGAGC |
| | TCATTCGAGACTACAACAGCAGTCGCGAGGACAGCCTGCAGGATGCCTGGGACTACGT |
| | GCAGGCTCAGGTCAAGTGCTGCGGCTGGGTCAGCTTCTACAACTGGACAGACAACGCT |
| | GAGCTCATGAATCGCCCTGAGGTCACCTACCCCTGTTCCTGCGAAGTCAAGGGGGAAG |
| | AGGACAACAGCCTTTCTGTGAGGAAGCGCTTCTGCGAGGCCCCCGGCAACAGGACCCA |
| | GAGTGGCAACCACCCTGAGGACTCGCCTGTGTACCAGGAGGGCTGCATGGAGAAGGTG |
| | CAGGCGTGGCTGCAGGAGAACCTGGCCATCATCCTCGGCGTGGGCGTGGGTGTCGCCA |
| | TCGTCGAOCTCCTGGGGATGGTCCTGTCCATCTGCTTGTGCCGGCACGTCCATTCCGA |
| | ACACTACAGCAAGGTCCCCAAGTACTGAG |

| | |
|---|---|
| ORF Start: ATG at 37 | ORF Stop: TGA at 838 |

| | | |
|---|---|---|
| SEQ ID NO: 6 | 267 aa | MW at 29611.2 kD |

| | |
|---|---|
| NOV2a, | MGSACIKVTKYFLFLFNLIFFILGAVILGFGVWILADKSSFISVLQTSSSSLRMGAYV |
| CG103322-01 | |
| Protein Sequence | FIGVGAVTMLMGFLGCIGAVNEVRCLLGLYFAFLLLILIAQVTAGALFYFNMGKLKQE |
| | MGGIVTELIRDYNSSREDSLQDAWDYVQAQVKCCGWVSFYNWTDNAELMNRPEVTYPC |
| | SCEVKGEEDNSLSVRKGFCEAPCNRTQSGNHPEDWPVYQEGCMEKVQAWLQENLGIIL |
| | GVGVGVAIVELLGMVLSICLCRHVHSEDYSKVPKY |

| | |
|---|---|
| SEQ ID NO: 7 | 747 bp |

| | |
|---|---|
| NOV2b | CCTTGGGATGGGCTCAGCCTGTATCAAAGTCACCAAATACTTTCTCTTCCTCTTCAAC |
| CG103322-02 DNA | |
| Sequence | TTGATCTTCTTTATCCTGGGCGCAGTGATCCTGGGCTTCGGGGTGTGGATCCTGGCCG |
| | ACAAGAGCACTTTCATCTCTGTCCTCCAAACCTCCTCCAGCTCGCTTAGGATGGGGGC |
| | CTATGTCTTCATCGGCGTGGGGGCAGTCACTATGCTCATGGGCTTCCTGGGCTGCATC |
| | GGCGCCGTCAACGAGGTCCGCTGCCTGCTGGGGCTGTACTTTGCTTTCCTGCTCCTGA |
| | TCCTCATTGCCCAGGTGACGGCCGCGGCCCTCTTCTACTTCAACATGGGCAAGCTGAA |
| | GCAGGAGATGGGTGGCATCGTCACTGAGCTCATTCGAGACTACAACAGCAGTCGCGAG |
| | GACACCCTGCAGGATGCCTGGGACTACGTGCAGGCTCAGGTGAAGTGCTGCGGCTGGG |
| | TCAGCTTCTACAACTGGACAGACAACGCTGAGCTCATGAATCGCCCTGAGGTCACCTA |
| | CCCCTGTTCCTGCGAAGTCAAGGGGGAAGAGGACAACAGCCTTTCTGTGAGGAAGGGC |
| | TTCTGCGAGGCCCCCGGCAACAGGACCCAGAGTGGCAACCACCCTGAGGACTGGCCTG |
| | TGTACCAGGAGCTCCTGGGGATGGTCCTGTCCATCTGCTTGTGCCGGCACGTCCATTC |
| | CGAAGACTACAGCAAGGTCCCCAAGTACTGAGGCAGCTGCTATCCCCATCT |

| | |
|---|---|
| ORF Start: ATG at 8 | ORF Stop: TGA at 725 |

| | | |
|---|---|---|
| SEQ ID NO: 8 | 239 aa | MW at 26702.7 kD |

| | |
|---|---|
| NOV2b, | MGSACIKVTKYFLFLFNLIFFILGAVILGFGVWILADKSSFISVLQTSSSSLRMGAYV |
| CG103322-02 | |
| Protein Sequence | FIGVGAVTMLMGFLGCIGAVNEVRCLLGLYFAFLLLILIAQVTAGALFYFNMGKLKQE |
| | MGGIVTELIRDYNSSREDSLQDAWDYVQAQVKCCGWVSFYNWTDNAELMNRPEVTYPC |
| | SCEVKGEEDNSLSVRKGFCEAPGNRTQSGNHPEDWPVYQELLGMVLSICLCRHVHSED |
| | YSKVPKY |

Sequences comparison of the above protein sequences yields the following sequence relationships shown in Table 2B.

TABLE 2B
Comparison of NOV2a against NOV2b.

| | | Identities/ |
|---|---|---|
| Protein | NOV2a Residues/ | Similarities for |
| Sequence | Match Residues | the Matched Region |
| NOV2b | 1 . . . 267 | 239/267 (89%) |
| | 1 . . . 239 | 239/267 (89%) |

Further analysis of the NOV2a protein yielded the following properties shown in Table 2C.

TABLE 2C
Protein Sequence Properties NOV2a

| | |
|---|---|
| PSort | 0.6400 probability located in plasma membrane; |
| analysis: | 0.4600 probability located in Golgi body; 0.3700 |
| | probability located in endoplasmic reticulum |
| | (membrane); 0.1000 probability located in |
| | endoplasmic reticulum (lumen) |
| SignalP | Cleavage site between residues 37 and 38 |
| analysis: | |

A search of the NOV2a protein against the Geneseq database, a proprietary database that contains sequences published in patents and patent pulication, yielded several homologous proteins shown in Table 2D.

TABLE 2D
Geneseq Results for NOV2a

| | | NOV2a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAM23963 | Human EST encoded protein SEQ | 1 . . . 267 | 266/267 (99%) | e−157 |
| | ID NO: 1488 - Homo sapiens, | 1 . . . 267 | 267/267 (99%) | |
| | 267 aa. [WO200154477-A2, 02 | | | |
| | AUG. 2001] | | | |
| AAW05732 | Human metastasis tumour | 1 . . . 267 | 266/267 (99%) | e−157 |
| | suppressor gene KAI1 product | 1 . . . 267 | 267/267 (99%) | |
| | [WO9634117-A1, 31 OCT. 1996] | | | |
| ABB57295 | Mouse ischaemic condition | 1 . . . 267 | 203/267 (76%) | e−120 |
| | related protein sequence SEQ | 1 . . . 266 | 230/267 (86%) | |
| | ID NO: 828 - Mus musculus, 266 | | | |
| | aa. [WO200188188-A2, 22 NOV. | | | |
| | 2001] | | | |
| AAB58792 | Breast and ovarian cancer | 1 . . . 117 | 110/117 (94%) | 4e−56 |
| | associated antigen protein | 69 . . . 185 | 112/117 (95%) | |
| | sequence SEQ ID 500 - Homo | | | |
| | sapiens, 198 aa. | | | |
| | WO200055173-A1, 21 SEP. 2000] | | | |
| AAG00436 | Human secreted protein, SEQ | 46 . . . 130 | 84/85 (98%) | 5e−41 |
| | ID NO: 4517 - Homo sapiens, | 15 . . . 99 | 85/85 (99%) | |
| | 99 aa. [EP1033401-A2, 06 SEP. | | | |
| | 2000] | | | |

In a BLAST search of public sequence datbases, the NOV2a protein was found to have homology to the proteins shown in the BLASTP data in Table 2E.

TABLE 2E
Public BLASTP Results for NOV2a

| | | NOV2a | Identities/ | |
|---|---|---|---|---|
| Protein | | Residues/ | Similarities | |
| Accession | | Match | for the Matched | Expect |
| Number | Protein/Organism/Length | Residues | Portion | Value |
| AAH00726 | Kangai 1 (suppression of | 1 . . . 267 | 267/267 (100%) | e−157 |
| | tumorigenicity 6, prostate, | 1 . . . 267 | 267/267 (100%) | |
| | CD82 antigen (R2 leukocyte | | | |
| | antigen, antigen detected by | | | |
| | monoclonal and antibody IA4)) | | | |
| | - Homo sapiens (Human), 267 | | | |
| | aa. | | | |
| P27701 | CD82 antigen (Inducible | 1 . . . 267 | 266/267 (99%) | e−157 |
| | membrane protein R2) (C33 | 1 . . . 267 | 267/267 (99%) | |
| | antigen) (IA4) (Metastasis | | | |
| | suppressor Kangai 1) | | | |
| | (Suppressor of | | | |
| | tumorigenicity-6) - Homo | | | |
| | sapiens (Human), 267 aa. | | | |
| P40237 | CD82 antigen (Inducible | 1 . . . 267 | 203/267 (76%) | e−119 |
| | membrane protein R2) (C33 | 1 . . . 266 | 230/267 (86%) | |
| | antigen) (IA4) - Mus musculus | | | |
| | (Mouse), 266 aa. | | | |
| O70352 | CD82 antigen (Metastasis | 1 . . . 267 | 202/267 (75%) | e−117 |
| | suppressor homolog) - Rattus | 1 . . . 266 | 226/267 (83%) | |
| | norvegicus (Rat), 266 aa. | | | |
| P11049 | Leukocyte antigen CD37 - Homo | 4 . . . 267 | 99/276 (35%) | 2e−45 |
| | sapiens (Human), 281 aa. | 6 . . . 280 | 159/276 (56%) | |

PFam analysis predicts that the NOV2a protein contains the domains shown in the Table 2F.

TABLE 2F
Domain Analysis of NOV2a

| | | Identities/ | |
|---|---|---|---|
| | NOV2a Match | Similarities for | |
| Pfam Domain | Region | the Matched Region | Expect Value |
| transmembrane4 | 10 . . . 256 | 102/270 (38%) | 2.6e−96 |
| | | 221/270 (82%) | |

### Example 3

The NOV3 clone was analyzed, and the nucleotide and encoded polypeptide sequences are shown in Table 3A.

TABLE 3A
NOV3 Sequence Analysis

| | |
|---|---|
| SEQ ID NO: 9 | 486 bp |

| | |
|---|---|
| NOV3a, | ATGGCAAAAGAGGAGCCCCAGAGTATCTCAAGGGACTTGCAGGAACTGCAGAAGAAGC |
| CG151575-01 DNA | |
| Sequence | TGTCTCTGCTGATAGACTCCTTCCAGAATAACTCAAAGGTGGTGGCCTTTATGAAGTC |
| | TCCAGTGGGTCAGTACTTGGACAGCCATCCGTTTCTGGCCTTCACCTTGCTGGTGTTC |
| | ATTGTCATGTCGGCCGTTCCTGTTGGATTCTTCCTGCTCATCGTGGTGCTTACCACCC |
| | TGGCTGCTCTGCTCGGGGTCATAATATTGGAAGGATTGGTCATCTCTGTGGGTGGCTT |
| | CTCACTGCTCTGCATCCTCTGTGGTTTGGGCTTCGTATCACTCGCCATGTCCGGGATG |
| | ATGATAGCATCTTATGTAGTGGTCTCCAGCCTCATCAGCTGCTGGTTTTCTCCCAGGC |
| | CACTGACACAGCAAAACACCAGTTGTGACTTTCTGCCAGCCATGAAGTCTGCAGACTT |
| | CGAGGGGCTTTACCAGGAATGA |

| | |
|---|---|
| ORF Start: ATG at 1 | ORF Stop: TGA at 484 |

| | | |
|---|---|---|
| SEQ ID NO: 10 | 161 aa | MW at 17507.6 kD |

| | |
|---|---|
| NOV3a, | MAKEEPQSISRDLQELQKKLSLLIDSFQNNSKVVAFMKSPVGQYLDSHPFLAFTLLVF |
| CG151575-01 | |
| Protein Sequence | IVMSAVPVGFFLLIVVLTTLAALLGVIILEGLVISVGGFSLLCILCGLGFVSLAMSGM |
| | MIASYVVVSSLISCWFSPRPLTQQNTSCDFLPAMKSADFEGLYQE |

| | |
|---|---|
| SEQ ID NO: 11 | 760 bp |

| | |
|---|---|
| NOV3b, | GGCTCCCTCTCGGGACGCTCTTTCCTTCTTCCTCTTGTTCCTCCTCCTGCCTCTCTTC |
| CG151575-02 DNA | |
| Sequence | GCTTCGCCTGCAAACGCGGTGGGGGCTGCTCGGCGGTCAGGAGCAGCAAGAGACAGAG |
| | CGACATGAGAGATTGGACCGCGGGCTGCACTGGACAATTTACTGGTAGGATAATTCAT |
| | CCCTAAAGAGATTGAAGTGAGCTTCAGAATGGCAAAAGAGGAGCCCCAGAGTATCTCA |
| | AGGGACTTGCAGGAACTGCACAAGAAGCTGTCTCTGCTGATAGACTCCTTCCAGAATA |
| | ACTCAAAGCTGCCCCAACACAGCAGGATCTCACTGGACTCTGATCATGGAGTGTCCAG |
| | GCTGGCCAGTGCTGGCTCCAAGGTGGTGGCCTTTATGAAGTCTCCAGTGGGTCAGTAC |
| | TTGGACAGCCATCCGTTTCTGGCCTTCACCTTGCTGGTGTTCATTGTCATGTCGGCCG |
| | TTCCTGTTGGATTCTTCCTGCTCATCGTGGTGCTTACCACCCTGGCTGCTCTGCTGGG |
| | GGTCATAATATTGGAAGGATTGGTCATCTCTGTCGGTGGCTTCTCACTGCTCTGCATC |
| | CTCTGTGGTTTGGGCTTCGTATCACTCGCCATGTCGGGGATGATGATAGCATCTTATG |
| | TAGTGGTCTCCAGCCTCATCAGCTGCTGGTTTTCTCCCAGGCCACTGACACAGCAAAA |
| | CACCAGTTGTGACTTTCTOCCAGCCATGAAGTCTGCAGACTTCGAGGGGCTTTACCAG |
| | GAATGA |

| | |
|---|---|
| ORF Start: ATG at 203 | ORF Stop: TGA at 758 |

| | | |
|---|---|---|
| SEQ ID NO: 12 | 185 aa | MW at 19972.2 kD |

| | |
|---|---|
| NOV3b, | MAKEEPQSISRDLQELQKKLSLLIDSFQNNSKLPQHSRISLDSDDGVSRLGSAGSKVV |
| CG151575-02 | |
| Protein Sequence | AFMKSPVGQYLDSHPFLAFTLLVFIVMSAVPVGFFLLIVVLTTLAALLGVIILEGLVI |
| | SVGGFSLLCILCGLGFVSLAMSGMMIASYVVVSSLISCWFSPRPLTQQNTSCDFLPAM |
| | KSADFEGLYQE |

Sequence comparison of the above protein sequences yields the following sequence relationships shown in Table 3B.

TABLE 3B
Comparison of NOV3a against NOV3b.

| | | Identities/ |
|---|---|---|
| Protein | NOV3a Residues/ | Similarities for |
| Sequence | Match Residues | the Matched Region |
| NOV3b | 1 . . . 161 | 161/185 (87%) |
| | 1 . . . 185 | 161/185 (87%) |

Further analysis of the NOV3a protein yielded the following properties shown in Table 3C.

TABLE 3C
Protein Sequence Properties NOV3a

| | |
|---|---|
| PSort | 0.6000 probability located in plasma membrane; |
| analysis: | 0.4000 probability located in Golgi body; 0.3000 |
| | probability located in endoplasmic reticulum |
| | (membrane); 0.0300 probability located in |
| | mitochondrial inner membrane |
| SignalP | Cleavage site between residues 69 and 70 |
| analysis: | |

A search of the NOV3a protein against the Geneseq database, a proprietary database that contains sequences published in patents and patent publication, yielded several homologous proteins shown in Table 3D.

TABLE 3D
Geneseq Results for NOV3a

| | | NOV3a | Identities/ | |
|---|---|---|---|---|
| | | Residues/ | Similarities | |
| Geneseq | Protein/Organism/Length | Match | for the Matched | Expect |
| Identifier | [Patent #, Date] | Residues | Region | Value |
| AAM93733 | Human polypeptide, SEQ ID | 1 . . . 161 | 161/161 (100%) | 9e−86 |
| | NO: 3697 - Homo sapiens, 161 | 1 . . . 161 | 161/161 (100%) | |
| | aa. [EP1130094-A2, 05 SEP. | | | |
| | 2001] | | | |
| ABG16996 | Novel human diagnostic | 47 . . . 133 | 24/89 (26%) | 0.93 |
| | protein #16987 - Homo | 280 . . . 366 | 53/89 (58%) | |
| | sapiens, 1076 aa. | | | |
| | [WO200175067-A2, 11 OCT. | | | |
| | 2001] | | | |
| ABP30247 | Streptococcus polypeptide | 64 . . . 114 | 23/56 (41%) | 1.2 |
| | SEQ ID NO 9670 - | 327 . . . 381 | 33/56 (58%) | |
| | Streptococcus agalactiae, | | | |
| | 401 aa. [WO200234771-A2, 02 | | | |
| | MAY 2002] | | | |
| ABP26074 | Streptococcus polypeptide | 64. .114 | 23/56 (41%) | 1.2 |
| | SEQ ID NO 1324 - | 334. .388 | 33/56 (58%) | |
| | Streptococcus agalactiae, | | | |
| | 408 aa. [WO200234771-A2, 02 | | | |
| | MAY 2002] | | | |
| ABB92972 | Herbicidally active | 58 . . . 123 | 22/68 (32%) | 3.6 |
| | polypeptide SEQ ID NO 2183 - | 175 . . . 236 | 37/68 (54%) | |
| | Arabidopsis thaliana, 436 | | | |
| | aa. [WO200210210-A2, 07 FEB. | | | |
| | 2002] | | | |

In a BLAST search of public sequence datbases, the NOV3a protein was found to have homology to the proteins shown in the BLASTP data in Table 3E.

TABLE 3E
Public BLASTP Results for NOV3a

| | | NOV3a | Identities/ | |
|---|---|---|---|---|
| Protein | | Residues/ | Similarities | |
| Accession | | Match | for the Matched | Expect |
| Number | Protein/Organism/Length | Residues | Portion | Value |
| Q96B96 | Similar to hypothetical | 1 . . . 161 | 161/161 (100%) | 3e−85 |
| | protein from clone 24796 - | 1 . . . 161 | 161/161 (100%) | |
| | Homo sapiens (Human), 161 | | | |
| | aa. | | | |
| O00323 | Hypothetical 17.6 kDa | 1 . . . 161 | 159/161 (98%) | 4e−84 |
| | protein - Homo sapiens | 1 . . . 161 | 160/161 (98%) | |
| | (Human), 161 aa. | | | |
| Q922Z1 | Similar to hypothetical | 1 . . . 158 | 112/159 (70%) | 5e−57 |
| | protein from clone 24796 - | 1 . . . 159 | 134/159 (83%) | |
| | Mus musculus (Mouse), 161 | | | |
| | aa. | | | |
| P43932 | Hypothetical protein HI0056 | 33 . . . 100 | 19/68 (27%) | 1.5 |
| | - Haemophilus influenzae, | 168 . . . 224 | 34/68 (49%) | |
| | 237 aa. | | | |
| Q9RZJ6 | Hypothetical protein | 33 . . . 96 | 20/67 (29%) | 2.0 |
| | DRB0131 - Deinococcus | 219 . . . 285 | 35/67 (51%) | |
| | radiodurans, 304 aa. | | | |

PFam analysis predicts that the NOV3a protein contains the domains shown in the Table 3F.

TABLE 3F
Domain Analysis of NOV3a

| | | | |
|---|---|---|---|
| | NOV3a Match | Identities/ for | |
| Pfam Domain | Region | the Matched Region | Expect Value |

No Significant Matches Found

### Example 4

The NOV4 clone was analyzed, and the nucleotide and encoded polypeptide sequences are shown in Table 4A.

TABLE 4A
NOV4 Sequence Analysis

| | |
|---|---|
| SEQ ID NO: 13 | 1088 bp |

| | |
|---|---|
| NOV4a, | GTGAGTTTACCCCTATGAGACTGTGAGAGOCCCGGGGCCTACCTCAAAGGAGCGGGGT |
| CG151608-01 DNA | |
| Sequence | CGCGAAGCTAGCTAGCAGCGGCCCCCCTCCAGGTCCCCGGGCCCGGCGGCGCGGCGGC |
| | GGCTTGGTTGTGAAGAGGCGGGGAAGCGGGTGTCCGGTCCCCGCCATGGAGGGCATGG |
| | ACGTAGACCTGGACCCGGAGCTGATGCAGAAGTTCAGCTGCCTGGGCACCACCGACAA |
| | GGACGTGCTCATCTCCGAGTTCCAGAGGCTGCTCGCCTTCCAGCTCAATCCTGCCGGT |
| | TGCGCCTTCTTCCTGCACATGACCAACTGGAACCTACAAGCAGCAATTGGCGCCTATT |
| | ATGACTTTGAGAOCCCAAACATCAGTGTGCCCTCTATGTCCTTTGTTGAAGATGTCAC |
| | CATAGGAGAAGCGGAGTCAATACCTCCGGATACTCAGTTTGTAAAAACATGGCGGATC |
| | CAGAATTCTGGGGCAGAGGCCTGGCCTCCAGGGGTTTGTCTTAAATATGTCGGGGGAG |
| | ACCAATTTGGACATGTGAACATGGTGATGGTGAGATCGCTAGAGCCCCAAGAGATTGC |
| | AGATGTCAGCGTCCAGATGTGCAGCCCCAGCAGAGCAGGAATGTATCAGGGACAGTGG |
| | CGGATGTGCACTGCTACAGGACTCTACTATGGAGATGTCATCTGGGTGATTCTCAGTG |
| | TGGAGCTGGGTGGACTTTTAGGAGTAACGCAGCAGCTGTCATCTTTTGAAACGGAGTT |
| | CAACACACAGCCGCATCGTAAGCTAGAAGGAAACTTCAACCCTTTTGCCTCTCCCCAA |
| | AACAACCGACAATCAGATGAAAACAACTTAAAAGACCCTGGGGGCTCCGAGTTCGACT |
| | CGATCAGCAAAAACACATGGGCTCCTGCTCCTGACACATGGGCTCCTGCTCCTGACCA |
| | AACTGAGCAAGACCAGAATAGACTGTCACAGAACTCTGTAAATCTGTCTCCCAGCAGT |
| | CACGCAAACAACTTATCAGTAGTGACTTACAGTAAGGGGCTCCATGGGCCTTACCCCT |
| | TCGGCCAGTCTTAAACGGGTGTCAGCAAAAAAAAAAAAAAAAAA |

| | |
|---|---|
| ORF Start: ATG at 162 | Stop: TAA at 1056 |

| | | |
|---|---|---|
| SEQ ID NO: 141 | 298 aa | MW at 32871.4 kD |

| | |
|---|---|
| NOV4a, | MEGMDVDLDPELMQKFSCLGTTDKDVLISEFQRLLGFQLNPAGCAFFLDMTNWNLQAA |
| CG151608-01 | |
| Protein Sequence | IGAYYDFESPNISVPSMSFVEDVTIGEGESIPPDTQFVKTWRIQNSGAEAWPPGVCLK |
| | YVGGDQFGHVNMVMVRSLEPQEIADVSVQMCSPSRAGMYQGQWRMCTATGLYYGDVIW |
| | VILSVEVGGLLGVTQQLSSFETEFNTQPHRKVEGNFNPFASPQKNRQSDENNLKDPGG |
| | SEFDSISKNTWAPAPDTWAPAPDQTEQDQNRLSQNSVNLSPSSHANNLSVVTYSKGLH |
| | CPYPFGQS |

| | |
|---|---|
| SEQ ID NO: 15 | 735 bp |

| | |
|---|---|
| NOV4b, | AGGCGGGGAAGCGGGTGTCCGGTCCCCGCCATGGAGGGCATGGACGTAGACCTGGACC |
| CG151608-02 DNA | |
| Sequence | CGGAGCTGATGCAGAAGTTCAGCTGCCTGGGCACCACCGACAAGGACGTGCTCATCTC |
| | CGAGTTCCAGAGGCTGCTCCOCTTCCAGCTCAATCCTGCCGGTTGCGCCTTCTTCCTG |
| | GACATGACCAACTGGAACCTACAAGCAGCAATTGGCGCCTATTATGACTTTGAGAGCC |
| | CAAACATCAGTGTGCCCTCTATGTCCTTTGTTGAAGATGTCACCATAGGAGAAGGGGA |
| | GTCAATACCTCCGGATACTCAGTTTGTAAAAACATGGCGGATCCAGAATTCTGATGTC |
| | ATCTGGGTGATTCTCAGTGTGGAGGTGGGTGCACTTTTAGGAGTAACGCAGCAGCTGT |
| | CATCTTTTGAAACOGAGTTCAACACACAGCCGCATCGTAAGGTAGAAGGAAACTTCAA |
| | CCCTTTTGCCTCTCCCCAAAAGAACCGACAATCAGATGAAAACAACTTAAAAGACCCT |
| | GGGGGCTCCGAGTTCGACTCGATCAGCAAAAACACATGGGCTCCTGCTCCTGACACAT |
| | GGGCTCCTGCTCCTGACCAAACTGAGCAAGACCAGAATAGACTGTCACAGAACTCTGT |
| | AAATCTGTCTCCCAGCAGTCACGCAAACAACTTATCAGTAGTGACTTACAGTAAGGGG |
| | CTCCATGGGCCTTACCCCTTCGGCCAGTCTTAAACGGGT |

| | |
|---|---|
| ORF Start: ATG at 31 | ORF Stop: TAA at 727 |

| | | |
|---|---|---|
| SEQ ID NO: 16 | 232 aa | MW at 25G73.1 kD |

| | |
|---|---|
| NOV4b, | MEGMDVDLDPELMQKFSCLGTTDKDVLISEFQRLLGFQLNPAGCAFFLDMTNWNLQAA |
| CG151608-02 | |
| Protein Sequence | IGAYYDFESPNISVPSMSFVEDVTIGEGESIPPDTQFVKTWRIQNSDVIWVILSVEVG |
| | GLLGVTQQLSSFETEFNTQPHRKVEGNFNPFASPQKNRQSDENNLKDPGGSEFDSISK |
| | NTWAPAPDTWAPAPDQTEQDQNRLSQNSVNLSPSSHANNLSVVTYSKGLHGPYPFGQS |

Sequence comparison of the above protein sequences yields the following sequence relationships shown in Table 4B.

TABLE 4B
Comparison of NOV4a against NOV4b.

| | | Identities/ |
|---|---|---|
| Protein | NOV4a Residues/ | Similarities for |
| Sequence | Match Residues | the Matched Region |
| NOV4b | 171 . . . 298 | 128/128 (100%) |
| | 105 . . . 232 | 128/128 (100%) |

Further analysis of the NOV4a protein yielded the following properties shown in Table 4C.

TABLE 4C
Protein Sequence Properties NOV4a

| | |
|---|---|
| PSort | 0.7000 probability located in plasma membrane; |
| analysis: | 0.3389 probability located in microbody |
| | (peroxisome); 0.2000 probability located in |
| | endoplasmic reticulum (membrane); 0.1000 |
| | probability located in mitochondrial inner membrane |
| SignalP | No Known Signal Sequence Predicted |
| analysis: | |

A search of the NOV4a protein against the Geneseq database, a proprietary database that contains sequences published in patents and patent publication, yielded several homologous proteins shown in Table 4D.

TABLE 4D
Geneseq Results for NOV4a

| | | NOV4a | Identities/ | |
|---|---|---|---|---|
| | | Residues/ | Similarities | |
| Geneseq | Protein/Organism/Length | Match | for the Matched | Expect |
| Identifier | [Patent #, Date] | Residues | Region | Value |
| ABG40261 | Human peptide encoded by | 172 . . . 287 | 116/116 (100%) | 2e−63 |
| | genome-derived single exon | 1 . . . 116 | 116/116 (100%) | |
| | probe SEQ ID 29926 - Homo | | | |
| | sapiens, 116 aa. | | | |
| | [WO200186003-A2, 15 NOV. | | | |
| | 2001] | | | |
| AAM18432 | Peptide #4866 encoded by | 172 . . . 287 | 116/116 (100%) | 2e−63 |
| | probe for measuring cervical | 1 . . . 116 | 116/116 (100%) | |
| | gene expression - Homo | | | |
| | sapiens, 116 aa. | | | |
| | [WO200157278-A2, 09 AUG. | | | |
| | 2001] | | | |
| AAM58143 | Human brain expressed single | 172 . . . 287 | 116/116 (100%) | 2e−63 |
| | exon probe encoded protein | 1 . . . 116 | 116/116 (100%) | |
| | SEQ ID NO: 30248 - Homo | | | |
| | sapiens, 116 aa. | | | |
| | [WO200157275-A2, 09 AUG. | | | |
| | 2001] | | | |
| ABB22766 | Protein #4765 encoded by | 172 . . . 287 | 116/116 (100%) | 2e−63 |
| | probe for measuring heart | 1 . . . 116 | 116/116 (100%) | |
| | cell gene expression - Homo | | | |
| | sapiens, 116 aa. | | | |
| | [WO200157274-A2, 09 AUG. | | | |
| | 2001] | | | |
| ABG15581 | Novel human diagnostic | 1 . . . 83 | 83/83 (100%) | 7e−43 |
| | protein #15572 - Homo | 44 . . . 126 | 83/83 (100%) | |
| | sapiens, 139 aa. | | | |
| | [WO200175067-A2, 11 OCT. | | | |
| | 2001] | | | |

In a BLAST search of public sequence datbases, the NOV4a protein was found to have homology to the proteins shown in the BLASTP data in Table 4E.

TABLE 4E
Public BLASTP Results for NOV4a

| | | NOV4a | Identities/ | |
|---|---|---|---|---|
| Protein | | Residues/ | Similarities | |
| Accession | | Match | for the Matched | Expect |
| Number | Protein/Organism/Length | Residues | Portion | Value |
| Q9BUR9 | Hypothetical 32.9 kDa | 1 . . . 298 | 298/298 (100%) | e−179 |
| | protein - Homo sapiens | 1 . . . 298 | 298/298 (100%) | |
| | (Human), 298 aa. | | | |
| Q96MG5 | CDNA FLJ32402 fis, clone | 171 . . . 298 | 128/128 (100%) | 2e−71 |
| | SKMUS2000343 - Homo sapiens | 105 . . . 232 | 128/128 (100%) | |
| | (Human), 232 aa. | | | |
| Q9VX56 | CG5445 protein (LD03052p) - | 5 . . . 176 | 65/172 (37%) | 8e−25 |
| | Drosophila melanogaster | 111 . . . 263 | 94/172 (53%) | |
| | (Fruit fly), 303 aa. | | | |
| Q9BL99 | Hypothetical 28.4 kDa | 8 . . . 179 | 52/184 (28%) | 2e−16 |
| | protein - Caenorhabditis | 4 . . . 186 | 92/184 (49%) | |
| | elegans, 245 aa. | | | |
| Q9SB64 | Hypothetical 76.2 kDa | 77 . . . 180 | 38/110 (34%) | 8e−13 |
| | protein - Arabidopsis | 380 . . . 487 | 58/110 (52%) | |
| | thaliana (Mouse-ear cress), | | | |
| | 704 aa. | | | |

PFam analysis predicts that the NOV4a protein contains the domains shown in the Table 4F.

TABLE 4F
Domain Analysis of NOV4a

| | | | |
|---|---|---|---|
| | | Identities/ | |
| | NOV4a Match | Similarities for | |
| Pfam Domain | Region | the Matched Region | Expect Value |

No Significant Matches Found

### Example 5

The NOV5 clone was analyzed, and the nucleotide and encoded polypeptide sequences are shown in Table 5A.

TABLE 5A
NOV5 Sequence Analysis

| | |
|---|---|
| SEQ ID NO: 17 | 3544 bp |

| | |
|---|---|
| NOV5a, | ACATGCCCCGTTTGCTGCCTGAACCTCTCCACAAAGACTCCCAGATCCTGAATTGAAT |
| CG152323-01 DNA | |
| Sequence | TTAATCATCTCCTGACAAAAGAATGCAATTTCAACTGACCCTTTTTTTGCACCTTGGG |
| | TGGCTCAGTTACTCAAAAGCTCAAGATGACTGCAACAGGGGTGCCTGTCATCCCACCA |
| | CTGGTGATCTCCTGGTGGGCAGGAACACGCAGCTTATGGCTTCTTCTACCTGTGGGCT |
| | GAGCAGAGCCCAGAAATACTGCATCCTCAGTTACCTGGAGGGGGAACAAAAATGCTCC |
| | ATCTGTGACTCTAGATTTCCATATGATCCGTATGACCAACCCAACAGCCACACCATTG |
| | AGAATGTCACTGTAAGTTTTGAACCAGACAGAGAAAAGAAATGGTGGCAATCTGAAAA |
| | TGGTCTTGATCATGTCACCATCAGACTGGACTTAGAGGCATTATTTCGGTTCAGCCAC |
| | CTTATCCTGACCTTTAAGACTTTTCGGCCTGCTGCAATGTTAGTTGAACGTTCCACAG |
| | ACTATGGACACAACTGGAAAGTGTTCAAATATTTTGCAAAAGACTGTGCCACTTCCTT |
| | TCCTAACATCACATCTGCCCAGGCCCAGGGAGTGGGAGACATTGTTTGTGACTCCAAA |
| | TACTCCGATATTGAACCCTCAACAGGTGGAGAGGTTGTTTTAAAAGTTTTGGATCCCA |
| | GTTTTGAAATTGAAAACCCTTATAGCCCCTACATCCAAGACCTTGTGACATTGACAAA |
| | CCTGAGGATAAACTTTACCAAGCTCCACACCCTTGGGGATGCTTTGCTTGGAAGCAGG |
| | CAAAATGATTCCCTTOATAAATACTACTATGCTCTGTACGAGATGATTGTTCGGGGAA |
| | GCTGCTTTTGCAATGGCCATGCTAGCGAATGTCGCCCTATGCAGAAGATGCCGGGAGA |
| | TGTTTTCAGCCCTCCTGGAATGGTTCACGGTCAGTGTGTGTGTCAGCACAATACAGAT |
| | GGTCCGAACTGTGAGAGATGCAAGGACTTCTTCCAGGATGCTCCTTGGAGGCCAGCTG |
| | CAGACCTCCAGGACAACGCTTGCAGATCGTGCAGCTGTAATAGCCACTCCAGCCGCTG |
| | TCACTTTGACATGACTACGTACCTGGCAACCGGTGGCCTCAGCGGGGGCGTGTGTGAA |
| | GACTGCCAGCACAACACTGAGGGGCAGCACTGCGACCGCTGCAGACCCCTCTTCTACA |
| | GGGACCCGCTCAAGACCATCTCAGATCCCTACGCGTGCATTCCTTGTGAATGTGACCC |
| | CGATGGGACCATATCTGGTGGCATTTGTGTGAGCCACTCTGATCCTGCCTTAGGGTCT |
| | GTGGCCGGCCAGTGCCTTTGTAAAGAGAACGTGGAAGGAGCCAAATGCGACCAGTGCA |
| | AACCCAACCACTACGGACTAAGCGCCACCGACCCCCTGGGCTGCCAGCCCTGCGACTG |
| | TAACCCCCTTGGGAGTCTGCCATTCTTGACCTGTGATGTGGATACAGGCCAATGCTTG |
| | TGCCTGTCATATGTCACCGGACCACACTGCGAAGAATGCACTGTTGGATACTGGGGCC |
| | TGGGAAATCATCTCCATGGGTGTTCTCCCTGTGACTGTGATATTGGAGGTGCTTATTC |
| | TAACGTGTGCTCACCCAACAATGGGCAGTGTGAATGCCGCCCACATGTCACTGGCCGT |
| | AGCTGCTCTGAACCAGCCCCTGGCTACTTCTTTGCTCCTTTGAATTTCTATCTCTACG |
| | AGGCAGAGGAAGCCACAACACTCCAAGGACTGGCGCCTTTGGGCTCGGAGACGTTTGG |
| | CCAGAGTCCTGCTGTTCACGTTGTTTTAGGAGAGCCAGTTCCTGGGAACCCTGTTACA |
| | TGGACTGGACCTGGATTTGCCAGGGTTCTCCCTGGGGCTGGCTTGAGATTTGCTGTCA |
| | ACAACATTCCCTTTCCTGTGGACTTCACCATTGCCATTCACTATGAAACCCAGTCTGC |
| | AGCTGACTGGACTGTCCAGATTGTGGTGAACCCCCCTGGAGGGAGTGAGCACTGCATA |
| | CCCAAGACTCTACAGTCAAAGCCTCAGTCTTTTGCCTTACCAGCGGCTACGAGAATCA |
| | TGCTGCTTCCCACACCCATCTGTTTAGAACCAGATGTACAATATTCCATAGATGTCTA |
| | TTTTTCTCAGCCTTTGCAAGGAGAGTCCCACGCTCATTCACATGTCCTGGTGGACTCT |
| | CTTGGCCTTATTCCCCAAATCAATTCATTGGAGAATTTCTGCAGCAAGCAGGACTTAG |
| | ATGAGTATCAGCTTCACAACTGTGTTGAAATTGCCTCAGCAATGGGACCTCAAGTGCT |
| | CCCGGGTGCCTGTGAAAGGCTGATCATCAGCATGTCTGCCAAGCTGCATGATGGGGCT |
| | GTGGCCTGCAAGTGTCACCCCCAGGGCTCAGTCGGATCCAGCTGCAGCCGACTTGGAG |
| | GCCAGTGCCAGTGTAAACCTCTTGTGGTCGGGCGCTGCTGTGACAGGTGCTCAACTGG |
| | AAGCTATGATTTGGGGCATCACGGCTGTCACCCATGTCACTGCCATCCTCAAGGATCA |
| | AAGGACACTGTATGTGACCAAGTAACAGGACAGTGCCCCTGCCATGGAGAGGTGTCTG |
| | GCCGCCGCTGTGATCGCTGCCTGGCAGGCTACTTTGGATTTCCCAGCTGCCACCCTTG |
| | CCCTTGTAATAGGTTTGCTGAACTTTGTGATCCTGAGACAGGGTCATGCTTCAATTGT |
| | GGAGGCTTTACAACTGGCAGAAACTGTGAAAGGTGTATTGATGGTTACTATGGAAATC |
| | CTTCTTCAGGACAGCCCTGTCGTCCTTGCCTGTGTCCAGATGATCCCTCAACCAATCA |
| | GTATTTTGCCCATTCCTGTTATCAGAATCTGTGGAGCTCAGATGTAATCTGCAATTGT |
| | CTTCAAGGTTATACGGGTACTCAGTGTGGAGAATGCTCTACTGGTTTCTATGGAAATC |
| | CAAGAATTTCAGGAGCACCTTGCCAACCATGTGCCTGCAACAACAACATAGATGTAAC |
| | CGATCCAGAGTCCTGCAGCCGGGTAACAGGGGAGTCCCTTCGATGTTTGCACAACACT |
| | CAGGGCGCAAACTGCCAGCTCTGCAAACCAGGTCACTATGGATCAGCCCTCAATCAGA |
| | CCTGCAGAAGATGCTCCTGCCATGCTTCCGGCGTGAGTCCCATGGAGTGTCCCCCTGG |
| | TGGGGGAGCTTGCCTCTGTGACCCTGTCACTGGTGCATGTCCTTGTCTGCCGAATGTC |
| | ACAGGCCTGGCCTGTGACCGTTGTGCTGATGGATACTGGAATCTGGTCCCTGGCAGAG |
| | GATGTCAGTCATGTGACTGTGACCCTAGGACCTCTCAAAGTAGCCACTGTGACCAGGC |
| | AAGATACTTTAAAGCTTACTAGTGCACTCAAAGTGAGCATGATAGTGAGACATGGTTT |
| | CTAAATGTGTAAAGAAAGTTTCTTTTATGTACTGTTGTTAATTAGTGCATTGAAACAG |
| | GGGTGGCCTTACAGGGGATGGAGTCAGCCTCTATCAAGGAATGAAAACCAAAAAAAGA |
| | GAATGA |

| | |
|---|---|
| ORF Start: ATG at 81 | ORF Stop: TAG at 3384 |

| | | |
|---|---|---|
| SEQ ID NO: 18 | 1101 aa | MW at 119568.2 kD |

| | |
|---|---|
| NOV5a, | MQFQLTLFLHLGWLSYSKAQDDCNRGACHPTTGDLLVGRNTQLMASSTCGLSRAQKYC |
| CG152323-01 | |
| Protein Sequence | ILSYLEGEQKCSICDSRFPYDPYDQPNSHTIENVTVSFEPDREKKWWQSENGLDHVSI |
| | RLDLEALFRFSHLILTFKTFRPAANLVERSTDYGHNWKVFKYFAKDCATSFPNITSGQ |
| | AQGVGDIVCDSKYSDIEPSTGGEVVLKVLDPSFEIENPYSPYIQDLVTLTNLRINFTK |
| | LHTLGDALLGRRQNDSLDKYYYALYEMIVRGSCFCNGHASECRPMQKMRGDVFSPPGM |
| | VHGQCVCQHNTDGPNCERCKDFFQDAPWRPAADLQDNACRSCSCNSHSSRCHFDMTTY |
| | LASGGLSGGVCEDCQHNTEGQHCDRCRPLFYRDPLKTISDPYACIPCECDPDGTISGG |
| | ICVSHSDPALGSVAGQCLCKENVEGAKCDQCKPNHYGLSATDPLGCQPCDCNPLGSLP |
| | FLTCDVDTGQCLCLSYVTGAHCEECTVGYWGLGNHLHGCSPCDCDIGGAYSNVCSPKN |
| | GQCECRPHVTGRSCSEPAPGYFFAPLNFYLYEAEEATTLQGLAPLGSETFGQSPAVHV |
| | VLGEPVPGNPVTWTGPGFARVLPGAGLRFAVNNIPFPVDFTIAIHYETQSAADWTVQI |
| | VVNPPGGSEHCIPKTLQSKPQSFALPAATRIMLLPTPICLEPDVQYSIDVYFSQPLQG |
| | ESHAHSHVLVDSLGLIPQINSLENFCSKQDLDEYQLHNCVEIASAMGPQVLPGACERL |
| | IISMSAKLHDGAVACKCHPQGSVGSSCSRLGGQCQCKPLVVGRCCDRCSTGSYDLGHH |
| | GCHPCHCHPQGSKDTVCDQVTGQCPCHGEVSGRRCDRCLAGYFGFPSCHPCPCNRFAE |
| | LCDPETGSCFNCGGFTTGRNCERCIDGYYGNPSSGQPCRPCLCPDDPSSNQYFAHSCY |
| | QNLWSSDVICNCLQGYTGTQCGECSTGFYGNPRISGAPCQPCACNNNIDVTDPESCSR |
| | VTGECLRCLHNTQGANCQLCKPGHYGSALNQTCRRCSCHASGVSPMECPPGGGACLCD |
| | PVTGACPCLPNVTGLACDRCADGYWNLVPGRGCQSCDCDPRTSQSSHCDQARYFKAY |

Further analysis of the NOV5a protein yielded the following properties shown in Table 5B.

TABLE 5B
Protein Sequence Properties NOV5a

| | |
|---|---|
| PSort | 0.4500 probability located in cytoplasm; |
| analysis: | 0.3000 probability located in microbody |
| | (peroxisome); 0.1000 probability located in |
| | mitochondrial matrix space; 0.1000 probability |
| | located in lysosome (lumen) |
| SignalP | Cleavage site between residues 20 and 21 |
| analysis: | |

A search of the NOV5a protein against the Geneseq database, a proprietary database that contains sequences published in patents and patent publication, yielded several homologous proteins shown in Table 5C.

TABLE 5C
Geneseq Results for NOV5a

| | | NOV5a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAY15457 | Human laminin beta 4 | 1 . . . 1094 | 1094/1094 (100%) | 0.0 |
| | protein - Homo sapiens, | 1 . . . 1094 | 1094/1094 (100%) | |
| | 1761 aa. [WO9919348-A1, 22 | | | |
| | APR. 1999] | | | |
| AAY15459 | SEQ ID 5 of WO9919347 - | 1 . . . 1101 | 1094/1105 (99%) | 0.0 |
| | Homo sapiens, 1105 aa. | 1 . . . 1105 | 1094/1105 (99%) | |
| | [WO9919348-A1, 22 APR. 1999] | | | |
| AAM48896 | Laminin protein - | 23 . . . 1094 | 539/1089 (49%) | 0.0 |
| | Unidentified, 1786 aa. | 30 . . . 1098 | 707/1089 (64%) | |
| | [WO200193897-A2, 13 DEC. | | | |
| | 2001] | | | |
| ABB81591 | Human laminin 10 second | 23 . . . 1094 | 539/1089 (49%) | 0.0 |
| | chain protein sequence SEQ | 9 . . . 1077 | 707/1089 (64%) | |
| | ID NO: 8 - Homo sapiens, | | | |
| | 1765 aa. [WO200250111-A2, | | | |
| | 27 JUN. 2002] | | | |
| ABB81590 | Human laminin 10 second | 23 . . . 1094 | 539/1089 (49%) | 0.0 |
| | chain protein sequence SEQ | 30 . . . 1098 | 707/1089 (64%) | |
| | ID NO: 6 - Homo sapiens, | | | |
| | 1786 aa. [WO200250111-A2, | | | |
| | 27 JUN. 2002] | | | |

In a BLAST search of public sequence datbases, the NOV5a protein was found to have homology to the proteins shown in the BLASTP data in Table 5D.

TABLE 5D
Public BLASTP Results for NOV5a

| | | NOV5a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9Y6U6 | WUGSC:H_RG015P03.1 protein - | 23 . . . 1093 | 1059/1071 (98%) | 0.0 |
| | Homo sapiens (Human), | 1 . . . 1069 | 1061/1071 (98%) | |
| | 1631 aa (fragment). | | | |
| Q9UHI2 | Laminin beta 1 related | 13 . . . 767 | 746/760 (98%) | 0.0 |
| | protein - Homo sapiens | 1 . . . 760 | 747/760 (98%) | |
| | (Human), 761 aa (fragment). | | | |
| O57484 | Laminin beta 2-like chain - | 23 . . . 1094 | 542/1084 (50%) | 0.0 |
| | Gallus gallus (Chicken), | 42 . . . 1103 | 712/1084 (65%) | |
| | 1792 aa. | | | |
| AAM61767 | Laminin beta 1 - | 21 . . . 1094 | 537/1092 (49%) | 0.0 |
| | Brachydanio rerio | 24 . . . 1095 | 712/1092 (65%) | |
| | (Zebrafish) (Danio rerio), | | | |
| | 1785 aa. | | | |
| CAC17320 | Sequence 15 from Patent | 23 . . . 1094 | 539/1089 (49%) | 0.0 |
| | WO0066730 - Homo sapiens | 9 . . . 1077 | 707/1089 (64%) | |
| | (Human), 1765 aa | | | |
| | (fragment). | | | |

PFam analysis predicts that the NOV5a protein contains the domains shown in the Table 5E.

TABLE 5E
Domain Analysis of NOV5a

| | | Identities/ | |
|---|---|---|---|
| | NOV5a Match | Similarities for | |
| Pfam Domain | Region | the Matched Region | Expect Value |
| laminin_Nterm | 28 . . . 263 | 114/266 (43%) | 6.8e−104 |
| | | 181/266 (68%) | |
| laminin_EGF | 265 . . . 329 | 18/71 (25%) | 1.5e−09 |
| | | 48/71 (68%) | |
| laminin_EGF | 332 . . . 392 | 20/65 (31%) | 4.8e−18 |
| | | 48/65 (74%) | |
| laminin_EGF | 395 . . . 452 | 27/60 (45%) | 4.5e−19 |
| | | 45/60 (75%) | |
| laminin_EGF | 455 . . . 503 | 28/59 (47%) | 1.7e−14 |
| | | 39/59 (66%) | |
| laminin_EGF | 506 . . . 548 | 20/59 (34%) | 0.00014 |
| | | 30/59 (51%) | |
| laminin_EGF | 769 . . . 814 | 24/59 (41%) | 4.5e−11 |
| | | 36/59 (61%) | |
| laminin_EGF | 817 . . . 860 | 23/59 (39%) | 8e−14 |
| | | 37/59 (63%) | |
| laminin_EGF | 863 . . . 908 | 25/59 (42%) | 6.4e−09 |
| | | 35/59 (59%) | |
| laminin_EGF | 911 . . . 967 | 16/62 (26%) | 0.00078 |
| | | 36/62 (58%) | |
| laminin_EGF | 970 . . . 1019 | 21/60 (35%) | 1.4e−14 |
| | | 38/60 (63%) | |
| laminin_EGF | 1022 . . . 1077 | 24/61 (39%) | 2.5e−12 |
| | | 41/61 (67%) | |

### Example 6

The NOV6 clone was analyzed, and the nucleotide and encoded polypeptide sequences are shown in Table 6A.

TABLE 6A
NOV6 Sequence Analysis

| | |
|---|---|
| SEQ ID NO: 19 | 2265 bp |

| | |
|---|---|
| NOV6a, | ACTTCCAGGTGGGAGTGCGTGGGCGGGGGAGCTGGAGCCGAGCGCCGCCGCCGAAGCT |
| CG153011-01 DNA | |
| Sequence | TCCGTCTCGCTCGCTCGCGCAGCGGCGGCAGCAGAGGTCGCGCACAGATGCGGGTTAG |
| | ACTGGCGGGGGGAGGAGGCGGAGGAGGGAAGGAAGCTGCATGCATGAGACCCACAGCG |
| | CAGAGAAATCTCACTGGGGACTGGGGCAGTAGGATCGATCCCAATCCCGAGGAAAACC |
| | AGAGAAGTAGCTGGGGGAGACGGTGCCACATTACTCTTGCAAGCTGGATGCCCTCTGT |
| | GGATGAAAGATGTATCATGGAATGAACCCGAGCAATGGAGATGGATTTCTAGAGCAGC |
| | AGCAGCAGCAGCAGCAACCTCAGTCCCCCCAGAGACTCTTGGCCGTGATCCTGTGGTT |
| | TCAGCTGGCGCTGTGCTTCGGCCCTGCACAGCTCACGGGCGCACCGGGGTGGCCTCAA |
| | GAAACTCACAATCATGGCAGAAGGGAAAGCAAACGCATCCTTCTTCACATGGCGGCAG |
| | CAAGGAGAAGTGCAGAGAGAGAAGAAAGTCCCTTATAAAACCATCAGACCTCATGAGA |
| | ACTCATTCACTATCACAAGAACAGCATGGAGGGTTCGATGACCTTCAAGTGTGTGCTG |
| | ACCCCGGCATTCCCGAGAATGGCTTCAGGACCCCCAGCGGAGGGGTTTTCTTTGAAGG |
| | CTCTGTAGCCCGATTTCACTGCCAAGACGGATTCAAGCTGAAGCGCGCTACAAAGAGA |
| | CTGTGTTTGAAGCATTTTAATGGAACCCTAGGCTGGATCCCAAGTGATAATTCCATCT |
| | GTGTGCAAGAAGATTGCCGTATCCCTCAAATCGAAGATGCTGAGATTCATAACAAGAC |
| | ATATAGACATGGAGAGAAGCTAATCATCACTTGTCATGAAGGATTCAAGATCCGGTAC |
| | CCCGACCTACACAATATGGTTTCATTATGTCGCGATGATGGAACGTGGAATAATCTGC |
| | CCATCTGTCAAGGCTGCCTCAGACCTCTAGCCTCTTCTAATGGCTATGTAAACATCTC |
| | TGAGCTCCAGACCTCCTTCCCGGTGGGGACTGTGATCTCCTATCGCTGCTTTCCCGGA |
| | TTTAAACTTGATGGGTCTCCGTATCTTGAGTGCTTACAAAACCTTATCTCGTCGTCCA |
| | GCCCACCCCGGTGCCTTGCTCTGGAAGCCCAAGTCTGTCCACTACCTCCAATGGTGAG |
| | TCACGGACATTTCGTCTCCCACCCGCGGCCTTGTGAGCGCTACAACCACGGAACTGTG |
| | GTGGAGTTTTACTGCGATCCTGGCTACAGCCTCACCAGCGACTACAAGTACATCACCT |
| | GCCAGTATGGAGAGTGGTTTCCTTCTTATCAAGTCTACTGCATCAAATCAGAGCAAAC |
| | GTGGCCCAGCACCCATGAGACCCTCCTGACCACGTGGAAGATTGTGGCGTTCACGGCA |
| | ACCAGTGTGCTGCTGGTGCTGCTGCTCGTCATCCTGGCCAGGATGTTCCAGACCAAGT |
| | TCAAGGCCCACTTTCCCCCCAOGGGGCCTCCCCGGAGTTCCAGCAGTGACCCTGACTT |
| | TGTGGTGGTAGACGGCGTGCCCGTCATGCTCCCGTCCTATGACGAAGCTGTGAGTGGC |
| | GGCTTGAGTGCCTTAGGCCCCGGGTACATGGCCTCTGTGGGCCAGGGCTGCCCCTTAC |
| | CCGTGGACGACCAGAGCCCCCCAGCATACCCCGGCTCAGGGGACACGGACACAGGCCC |
| | AGGGGAGTCAGAAACCTGTGACAGCGTCTCAGGCTCTTCTGAGCTGCTCCAAAGTCTG |
| | TATTCACCTCCCAGGTGCCAAGAGAGCACCCACCCTACTTCGGACAACCCTGACATAA |
| | TTGCCAGCACGGCAGAGGACGTGGCATCCACCAGCCCAGGCATCGACATTGCAGATGA |
| | GATTCCTCTAATGGAAGAAGATCCCTAATATGGGTCAAGATCCAGATGACTCTCCTGC |
| | TCCTTCGGGGAAAGGACCTTGTATCTTGGAGTGAGGTCACAGAAGGATAGAGCCTGGG |
| | GGCAAAATGTCTAACTTGTCTACATGGGGACCACAGTTCACATTATGCATCTCAGGCT |
| | CCACAGTGAGGCTGACAAACTGCAATGGCAGTGCTTTTAAATGAGATTTGAGGATTCA |
| | CCAAGACCCATGGGGAACCGGGOCAGCAGGGAAGCCCTCGCGTGGTCTTGGATGAGGG |
| | GTGTTAAATGTGTATCGTGCTGTGGAACATGGGACAATTCCACGCACTCCCACCTGGA |
| | AGT |

| | |
|---|---|
| ORF Start: ATG at 293 | ORF Stop: TAA at 1940 |

| | | |
|---|---|---|
| SEQ ID NO: 20 | 549 aa | MW at 60114.0 kD |

| | |
|---|---|
| NOV6a | MKDVSWNEPEQWRWISRAAAAAAATSVPPETLGRDPVVSAGAVLRPCTAHGRTGVASR |
| CG153011-01 | |
| Protein Sequence | NSQSWQKGKQTHPSSHGGSKEKCRERRKSLIKPSDLMRTHSLSQEQHGGFDDLQVCAD |
| | PGIPENGFRTPSGGVFFEGSVARFHCQDGFKLKGATKRLCLKHFNGTLGWIPSDNSIC |
| | VQEDCRIPQIEDAEIHNKTYRHGEKLIITCHEGFKIRYPDLHNMVSLCRDDGTWNNLP |
| | ICQGCLRPLASSNGYVNISELQTSFPVGTVISYRCFPGFKLDGSAYLECLQNLIWSSS |
| | PPRCLALEAQVCPLPPMVSHGDFVCHPRPCERYNHGTVVEFYCDPGYSLTSDYKYITC |
| | QYGEWFPSYQVYCIKSEQTWPSTHETLLTTWKIVAFTATSVLLVLLLVILARMFQTKF |
| | KAHFPPRGPPRSSSSDPDFVVVDGVPVMLPSYDEAVSGGLSALGPGYMASVGQGCPLP |
| | VDDQSPPAYPGSGDTDTGPGESETCDSVSGSSELLQSLYSPPRCQESTHPTSDNPDII |
| | ASTAEEVASTSPGIDIADEIPLMEEDP |

Further analysis of the NOV6a protein yielded the following properties shown in Table 6B.

TABLE 6B
Protein Sequence Properties NOV6a

| | |
|---|---|
| PSort | 0.8000 probability located in mitochondrial |
| analysis: | inner membrane; 0.7000 probability located in |
| | plasma membrane; 0.2000 probability located in |
| | endoplasmic reticulum (membrane); 0.0646 |
| | probability located in microbody (peroxisome) |
| SignalP | No Known Signal Sequence Predicted |
| analysis: | |

A search of the NOV6a protein against the Geneseq database, a proprietary database that contains sequences published in patents and patent publication, yielded several homologous proteins shown in Table 6C.

TABLE 6C
Geneseq Results for NOV6a

| | | NOV6a | Identities/ | |
|---|---|---|---|---|
| | | Residues/ | Similarities | |
| Geneseq | Protein/Organism/Length | Match | for the Matched | Expect |
| Identifier | [Patent #, Date] | Residues | Region | Value |
| AAB80234 | Human PRO222 protein - | 106 . . . 536 | 430/431 (99%) | 0.0 |
| | Homo sapiens, 490 aa. | 49 . . . 479 | 430/431 (99%) | |
| | [WO200104311-A1, | | | |
| | 18 JAN. 2001] | | | |
| AAU12326 | Human PRO222 | 106 . . . 536 | 430/431 (99%) | 0.0 |
| | polypeptide sequence - | 49 . . . 479 | 430/431 (99%) | |
| | Homo sapiens, 490 aa. | | | |
| | [WO200140466-A2, | | | |
| | 07 JUN. 2001] | | | |
| AAY13366 | Amino acid sequence | 106 . . . 536 | 430/431 (99%) | 0.0 |
| | of protein PRO222 - | 49 . . . 479 | 430/431 (99%) | |
| | Homo sapiens, 490 aa. | | | |
| | [WO9914328-A2, | | | |
| | 25 MAR. 1999] | | | |
| ABG26615 | Novel human diagnostic | 237 . . . 540 | 299/353 (84%) | e−175 |
| | protein #26606 - | 1 . . . 353 | 300/353 (84%) | |
| | Homo sapiens, 463 aa. | | | |
| | [WO200175067-A2, | | | |
| | 11 OCT. 2001] | | | |
| ABB55790 | Human polypeptide | 106 . . . 298 | 193/193 (100%) | e−117 |
| | SEQ ID NO 186 - | 49 . . . 241 | 193/193 (100%) | |
| | Homo sapiens, 290 aa. | | | |
| | [US2001039335-A1, | | | |
| | 08 NOV. 2001] | | | |

In a BLAST search of public sequence datbases, the NOV6a protein was found to have homology to the proteins shown in the BLASTP data in Table 6D.

TABLE 6D
Public BLASTP Results for NOV6a

| | | NOV6a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q95K70 | Hypothetical 43.3 | 157 . . . 549 | 376/393 (95%) | 0.0 |
| | kDa protein - Macaca | 1 . . . 393 | 384/393 (97%) | |
| | fascicularis | | | |
| | (Crab eating macaque) | | | |
| | (Cynomolgus | | | |
| | monkey), 393 aa. | | | |
| Q8VC43 | Hypothetical 43.1 kDa | 157 . . . 549 | 356/393 (90%) | 0.0 |
| | protein - Mus musculus | 1 . . . 393 | 372/393 (94%) | |
| | (Mouse), 393 aa. | | | |
| Q9BSR0 | Similar to hypothetical | 106 . . . 298 | 193/193 (100%) | e−117 |
| | protein FLJ10052 - | 49 . . . 241 | 193/193 (100%) | |
| | Homo sapiens (Human), | | | |
| | 290 aa. | | | |
| Q9NWG0 | Hypothetical 26.1 kDa | 106 . . . 242 | 137/137 (100%) | 8e−82 |
| | protein - Homo sapiens | 49 . . . 185 | 137/137 (100%) | |
| | (Human), 236 aa. | | | |
| Q92537 | Hypothetical protein | 299 . . . 491 | 83/206 (40%) | 2e−30 |
| | KIAA0247 - Homo sapiens | 39 . . . 241 | 114/206 (55%) | |
| | (Human), 303 aa. | | | |

PFam analysis predicts that the NOV6a protein contains the domains shown in the Table 6E.

TABLE 6E
Domain Analysis of NOV6a

| | | Identities/ | |
|---|---|---|---|
| | NOV6a Match | Similarities for | |
| Pfam Domain | Region | the Matched Region | Expect Value |
| sushi | 114 . . . 174 | 18/66 (27%) | 7.2e−07 |
| | | 44/66 (67%) | |
| sushi | 179 . . . 234 | 17/66 (26%) | 1.5e−10 |
| | | 47/66 (71%) | |
| sushi | 237 . . . 294 | 18/66 (27%) | 6.4e−13 |
| | | 43/66 (65%) | |
| sushi | 302 . . . 361 | 18/68 (26%) | 4e−08 |
| | | 44/68 (65%) | |

### Example 7

The NOV7 clone was analyzed, and the nucleotide and encoded polypeptide sequences are shown in Table 7A.

TABLE 7A
NOV7 Sequence Analysis

| | |
|---|---|
| SEQ ID NO: 21 | 1089 bp |

| | |
|---|---|
| NOV7a, | GGCTGTGGGTGCTTCACTATGGCGACGGTGGGGGCTCCGCGGCACTTCTCCCGCTGCG |
| CG153042-01 DNA | |
| Sequence | CCTGCTTCTGCACCGATAACTTGTACGTGGCGCGCTATGGGCTGCACGTGCGCTTCCG |
| | AGGCGAGCAGCAGCTGCCCCGGGACTACGGCCAGATCCTGCGCAGCCGAGGCTCTGTT |
| | AGCGCCAAGGACTTCCAGCAGCTGTTAGCAGACGTACTTGAGCAGGAGGTGGAGCGGC |
| | GGCAGCGGCTGGGGCAGGAGTCAGCAGCTAGGAAAGCCCTCATCGCGAGTTCCTACCA |
| | CCCGGCACGGCCTGAGGTCTACGACTCACTGCAGGATGCAGCTCTGGCCCCCGAGTTC |
| | CTGGCCGTGACTGAGTACAGCGTGTCCCCAGACGCAGACCTCAAGGGCCTTCTCCAGC |
| | GGCTGGAGACAGTATCGGAGGAGAACCGCATCTACCGGGTGCCTCTTTTCACAGCGCC |
| | CTTCTGCCAGGCCCTGCTGGAAGAGCTGGAGCACTTCGAGCAATCGGACATGCCTAAG |
| | GGGAGGCCCAACACCATGAACAACTACGGGGTGCTGCTGCACGAGCTCGGGCTGGACG |
| | AGCCGCTGATGACACCACTGCGGGAGCGCTTCCTGCAGCCGCTGATGGCCCTGCTGTA |
| | CCCTGACTGTGGCGGCGGCCGGCTCGACAGCCACCGGGCCTTTGTGGTCAAATACGCA |
| | CCGGGCCAGGACCTGGAGCTGGGCTGCCACTATGATAATGCCGAGCTCACCCTCAATG |
| | TGGCCTTGGGCAAGGTCTTCACAGGGGGCGCCCTGTATTTTGGGGGCCTCTTCCAGGC |
| | ACCCACAGCCCTGACGGAGCCCCTGGAGGTGGAGCACGTGGTGGGCCAGGGTGTCCTC |
| | CACCGTGGCGGCCADCTGCATGGAGCCCGGCCCTTGGGCACTGGTGAGCGTTGGAACC |
| | TTGTCGTCTGGCTCCGAGCCTCTGCTGTGCGCAACAGCCTCTGTCCCATGTGCTGCCG |
| | TGAGCCCGACCTGGTOGACGATGAGGGCTTCGGTGATGGCTTCACCCGAGAGGAGCCC |
| | GCCACGGTGGATGTATGTGCGCTCACCTGAGCTTGCTTGGGCCCA |

| | |
|---|---|
| ORF Start: ATG at 19 | ORF Stop: TGA at 1072 |

| | | |
|---|---|---|
| SEQ ID NO: 22 | 351 aa | MW at 39126.1 kD |

| | |
|---|---|
| NOV7a, | MATVGAPRHFCRCACFCTDNLYVARYGLHVRFRGEQQLRRDYGQILRSRGCVSAKDFQ |
| CG153042-01 | |
| Protein Sequence | QLLAEVLEQEVERRQRLGQESAARKALIASSYHPARPEVYDSLQDAALAPEFLAVTEY |
| | SVSPDADLKGLLQRLETVSEEKRIYRVPVFTAPFCQALLEELEHFEQSDMPKGRPNTM |
| | NNYGVLLHELGLDEPLMTPLRERFLQPLMALLYPDCGGGRLDSHRAFVVKYAPGQDLE |
| | LGCHYDNAELTLNVALGKVFTGGALYFGGLFQAPTALTEPLEVEHVVGQGVLHRGGQL |
| | HGARPLGTGERWNLVVWLRASAVRNSLCPMCCREPDLVDDEGFGDGFTREEPATVDVC |
| | ALT |

| | |
|---|---|
| SEQ ID NO: 23 | 1075 bp |

| | |
|---|---|
| NOV7b | CACCGGATCCACCATGGCGACGGTGGGGGCTCCGCGGCACTTCTGCCGCTGCGCCTCC |
| CG153042-02 DNA | |
| Sequence | TTCTGCACCGATAACTTGTACGTGGCGCGCTATGGGCTGCACGTGCGCTTCCGAGGCG |
| | AGCAGCAGCTGCGCCGGGACTACGGCCCGATCCTGCGCAGCCGAGGCTGTGTTAGCGC |
| | CAAGGACTTCCAGCAGCTGTTAGCAGAGCTTGAGCAGGAGGTGGAGCGGCGGCAGCGG |
| | CTGGGGCAGGAGTCAGCAGCTAGGAAAGCCCTCATCGCGAGTTCCTACCACCCGGCAC |
| | GGCCTGAGGTCTACGACTCACTGCAGGATGCAGCTCTGGCCCCCGAGTTCCTGGCCGT |
| | GACTGACTACAGCGTGTCCCCAGACGCAGACCTCAAGGGCCTTCTCCAGCGGCTGGAG |
| | ACAGTATCGGAGGAGAAGCGCATCTACCCGGTGCCTGTTTTCACAGCGCCCTTCTGCC |
| | AGGCCCTGCTGGAAGAGCTGGAGCACTTCGAGCAATCGGACATGCCTAAGGGGAGGCC |
| | CAACACCATGAACAACTACGCCGTGCTGCTGCACGAGCTCGGGCTGGACGAGCCCCTG |
| | ATGACACCACTGCGGGACCGCTTCCTGCAGCCGCTCATGGCCCTGCTGTACCCTGACT |
| | GTGGCGGGGGCCGGCTCGACAGCCACCGGGCCTTTGTGGTCAAATACGCACCGGGCCA |
| | GGACCTGGAGCTGGGCTGCCACTATGATAATGCCGAGCTCACCCTCAATGTGGCCTTG |
| | GGCAAGGTCTTCACAGGGGGCGCCCTGTATTTTGGGGGCCTCTTCCAGGCACCCACAG |
| | CCCTGACCGAGCCCCTGGAGGTGGAGCACGTGGTGGGCCAGCGTGTCCTCCACCGTGG |
| | CGGCCAGCTGCATGGAGCCCGGCCCTTGGGCACTGGTGAGCGTTGGAACCTTGTCGTC |
| | TGGCTCCGAGCCTCTGCTGTGCGCAACAGCCTCTGTCCCATGTGCTGCCGTGAGCCCG |
| | ACCTGGTGGACGATGAGGGCTTCGGTGATGGCTTCACCCGAGAGGAGCCCGCCACGGT |
| | GGATGTATGTGCGCTCACCTAGGTCGACGGC |

| | |
|---|---|
| ORF Start: ATG at 14 | ORF Stop: TAG at 1064 |

| | | |
|---|---|---|
| SEQ ID NO: 24 | 350 aa | MW at 38996.0 kD |

| | |
|---|---|
| NOV7b, | MATVGAPRHFCRCACFCTDNLYVARYGLHVRFRGEQQLRRDYGPILRSRGCVSAKDFQ |
| CG153042-02 | |
| Protein Sequence | QLLAELEQEVERRQRLGQESAARKALIASSYHPARPEVYDSLQDAALAPEFLAVTEYS |
| | VSPDADLKGLLQRLETVSEEKRIYRVPVFTAPFCQALLEELEHFEQSDMPKGRPNTMN |
| | NYGVLLHELGLDEPLMTPLRERFLQPLMALLYPDCGGGRLDSHRAFVVKYAPGQDLEL |
| | GCHYDNAELTLNVALGKVFTGGALYFGGLFQAPTALTEPLEVEHVVGQGVLHRGGQLH |
| | GARPLGTGERNNLVVWLRASAVRNSLCPMCCREPDLVDDEGFGDGFTREEPATVDVCA |
| | LT |

Sequence comparison of the above protein sequences yields the following sequence relationships shown in Table 7B.

TABLE 7B
Comparison of NOV7a against NOV7b.

| | | Identities/ |
|---|---|---|
| | NOV7a Residues/ | Similarities for |
| Protein Sequence | Match Residues | the Matched Region |
| NOV7b | 1 . . . 351 | 349/351 (99%) |
| | 1 . . . 350 | 349/351 (99%) |

Further analysis of the NOV7a protein yielded the following properties shown in Table 7C.

TABLE 7C
Protein Sequence Properties NOV7a

| | |
|---|---|
| PSort | 0.6500 probability located in plasma membrane; |
| analysis: | 0.4763 probability located in mitochondrial |
| | matrix space; 0.4500 probability located in |
| | cytoplasm; 0.2150 probability located |
| | in lysosome (lumen) |
| SignalP | Cleavage site between residues 12 and 13 |
| analysis: | |

A search of the NOV7a protein against the Geneseq database, a proprietary database that contains sequences published in patents and patent publication, yielded several homologous proteins shown in Table 7D.

TABLE 7D
Geneseq Results for NOV7a

| | | NOV7a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAB94678 | Human protein sequence | 65 . . . 351 | 287/287 (100%) | e−169 |
| | SEQ ID NO: 15628 - | 4 . . . 290 | 287/287 (100%) | |
| | Homo sapiens, | | | |
| | February 2001] | | | |
| AAG45676 | Arabidopsis thaliana | 86 . . . 314 | 87/238 (36%) | 2e−36 |
| | protein fragment | 27 . . . 256 | 126/238 (52%) | |
| | SEQ ID NO: 57373 - | | | |
| | Arabidopsis thaliana, | | | |
| | 310 aa. [EP1033405-A2, | | | |
| | 06 SEP. 2000] | | | |
| AAG45675 | Arabidopsis thaliana | 86 . . . 314 | 87/238 (36%) | 2e−36 |
| | protein fragment SEQ ID | 105 . . . 334 | 126/238 (52%) | |
| | NO: 57372 - Arabidopsis | | | |
| | thaliana, 388 aa. | | | |
| | [EP1033405-A2, | | | |
| | 06 SEP. 2000] | | | |
| AAG45674 | Arabidopsis thaliana | 86 . . . 314 | 87/238 (36%) | 2e−36 |
| | protein fragment | 114 . . . 343 | 126/238 (52%) | |
| | SEQ ID NO: 57371 - | | | |
| | Arabidopsis thaliana, | | | |
| | 397 aa. [EP1033405-A2, | | | |
| | 06 SEP. 2000] | | | |
| AAG06884 | Arabidopsis thaliana | 86 . . . 314 | 87/238 (36%) | 2e−36 |
| | protein fragment SEQ | 27 . . . 256 | 126/238 (52%) | |
| | ID NO: 3823 - | | | |
| | Arabidopsis thaliana, | | | |
| | 310 aa. [EP1033405-A2, | | | |
| | 06 SEP. 2000] | | | |

In a BLAST search of public sequence datbases, the NOV7a protein was found to have homology to the proteins shown in the BLASTP data in Table 7E.

TABLE 7E
Public BLASTP Results for NOV7a

| | | NOV7a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9CQ04 | 5730405M13Rik | 1 . . . 351 | 300/351 (85%) | e−175 |
| | protein - Mus musculus | 1 . . . 349 | 319/351 (90%) | |
| | (Mouse), 349 aa. | | | |
| Q9H8K6 | CDNA FLJ13491 fis, | 65 . . . 351 | 287/287 (100%) | e−168 |
| | clone PLACE1004274 - | 4 . . . 290 | 287/287 (100%) | |
| | Homo sapiens | | | |
| | (Human), 290 aa. | | | |
| Q9DBJ4 | 1300006G11Rik | 181 . . . 351 | 148/171 (86%) | 7e−85 |
| | protein (RIKEN cDNA | 1 . . . 171 | 157/171 (91%) | |
| | 1300006G11 gene) - | | | |
| | Mus musculus (Mouse), | | | |
| | 171 aa. | | | |
| Q93W24 | B1080D07.28 protein | 140 . . . 324 | 84/199 (42%) | 3e−37 |
| | (P0507H06.12 protein) - | 182 . . . 379 | 117/199 (58%) | |
| | Oryza sativa | | | |
| | (Rice), 404 aa. | | | |
| Q9LV19 | Gb\|AAB72163.1 | 86 . . . 314 | 82/239 (34%) | 1e−33 |
| | (Unknown protein) - | 122 . . . 351 | 125/239 (51%) | |
| | Arabidopsis thaliana | | | |
| | (Mouse-ear cress), | | | |
| | 394 aa. | | | |

PFam analysis predicts that the NOV7a protein contains the domains shown in the Table 7F.

TABLE 7F
Domain Analysis of NOV7a

| | | | |
|---|---|---|---|
| | | Identities/ | |
| | NOV7a Match | Similarities for | |
| Pfam Domain | Region | the Matched Region | Expect Value |

No Significant Matches Found

### Example 8

The NOV8 clone was analyzed, and the nucleotide and encoded polypepfide sequences are shown in Table 8A.

TABLE 8A
NOV8 Sequence Analysis

| | |
|---|---|
| SEQ ID NO: 25 | 1051 bp |

| | |
|---|---|
| NOV8a, | GAACCAGTAGCCGCGGCTGCTTCTGTTGCCCCGGTCGGTGGTCGTTATGGATTCTCCA |
| CG153179-01 DNA | |
| Sequence | TGGGACGAGTTGGCTCTGGCCTTCTCCCGCACGTCCATGTTTCCCTTTTTTGACATCG |
| | CGCACTATCTAGTGTCAGTGATGGCGGTGAAACGTCAGCCGGGAGCAGCTGCATTGGC |
| | ATGGAAGAATCCTATTTCAAGCTGGTTTACTGCTATGCTCCACTGTTTTGGTGGAGGA |
| | ATTTTATCCTGTCTACTGCTTOCAGAGCCTCCATTGAAGTTTCTTGCAAACCACACTA |
| | ACATATTACTGGCATCTTCAATCTGGTATATTACATTTTTTTGCCCGCATGACCTAGT |
| | TTCCCAGGGCTATTCATATCTACCTGTTCAACTACTGGCTTCGGGAATGAAGGAAGTG |
| | ACCAGAACTTGGAAAATAGTAGGTGGAGTCACACATGCTAATAGCTATTACAAAAATG |
| | GCTGGATAGTCATGATAGCTATTGGATGGGCCCGAGGTGCGGGTGGTACCATTATAAC |
| | GAATTTTGAGAGGTTGGTAAAAGGAGATTGGAAACCAGAAGGTCATGAATGGCTGAAG |
| | ACGTCATATTTTAGGGTACATGTGCAGAACGTGCAGGTTTGTTACATATGTATACATG |
| | TGCCATGTTGGTGTGCTACACCCATTAACTCGTCATTTAACATTAGCCCTGCCAAGGT |
| | AACCCTGCTGGGGTCAGTTATCTTCACATTCCAGCACACCCAGCATCTGGCAATATCA |
| | AAGCATAATCTTATGTTCCTTTATACCATCTTTATTGTGGCCACAAAGATAACCATGA |
| | TGACTACACAGACTTCTACTATGACATTTGCTCCTTTTGAGGATACATTGAGTTGGAT |
| | GCTATTTGGCTGGCAGCAGCCGTTTTCATCATGTGAGAAGAAAAGTGAAGCAAAGTCA |
| | CCTTCCAATGGCGTTGGGTCATTGGCCTCAAAGCCGGTAGATGTTGCCTCAGATAATG |
| | TTAAAAAGAAACATACTAAGAAGAATGAATAATTTACGTGATGAGCTCTACAAGGCCA |
| | AAAATTT |

| | |
|---|---|
| ORF Start: ATG at 47 | ORF Stop: TAA at 1016 |

| | | |
|---|---|---|
| SEQ ID NO: 26 | 323 aa | MW at 36140.7 kD |

| | |
|---|---|
| NOV8a, | MDSPWDELALAFSRTSMFPFFDIAHYLVSVMAVKRQPGAAALAWKNPISSWFTAMLHC |
| CG153179-01 | |
| Protein Sequence | FGGGILSCLLLAEPPLKFLANHTNILLASSIWYITFFCPHDLVSQGYSYLPVQLLASG |
| | MKEVTRTWKIVGGVTHANSYYKNGWIVMIAIGWARGAGGTIITNFERLVKGDWKPEGD |
| | EWLKTSYFRVHVQNVQVCYICIHVPCWCATPINSSFNISPAKVTLLGSVIFTFQHTQH |
| | LAISKHNLMFLYTIFIVATKITMMTTQTSTMTFAPFEDTLSWMLFGWQQPFSSCEKKS |
| | EAKSPSNGVGSLASKPVDVASDNVKKKHTKKNE |

Further analysis of the NOV8a protein yielded the following properties shown in Table 8B.

TABLE 8B
Protein Sequence Properties NOV8a

| | |
|---|---|
| PSort | 0.6000 probability located in plasma membrane; |
| analysis: | 0.4000 probability located in Golgi body; 0.3000 |
| | probability located in endoplasmic reticulum |
| | (membrane); 0.2397 probability |
| | located in mitochondrial inner membrane |
| SignalP | Cleavage site between residues 1 and 2 |
| analysis: | |

A search of the NOV8a protein against the Geneseq database, a proprietary database that contains sequences published in patents and patent publication, yielded several homologous proteins shown in Table 8C.

TABLE 8C
Geneseq Results for NOV8a

| | | NOV8a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAB92881 | Human protein | 1 . . . 323 | 290/323 (89%) | e−168 |
| | sequence SEQ ID NO: | 1 . . . 291 | 290/323 (89%) | |
| | 11479 - Homo sapiens, | | | |
| | 291 aa. [EP1074617-A2, | | | |
| | 07 FEB. 2001] | | | |
| AAM41733 | Human polypeptide | 1 . . . 323 | 290/323 (89%) | e−168 |
| | SEQ ID NO 6664 - | 13 . . . 303 | 290/323 (89%) | |
| | Homo sapiens, 303 aa. | | | |
| | [WO200153312-A1, | | | |
| | 26 JUL. 2001] | | | |
| AAM39947 | Human polypeptide | 1 . . . 323 | 290/323 (89%) | e−168 |
| | SEQ ID NO 3092 - | 1 . . . 291 | 290/323 (89%) | |
| | Homo sapiens, 291 aa. | | | |
| | [WO200153312-A1, | | | |
| | 26 JUL. 2001] | | | |
| ABB89884 | Human polypeptide | 1 . . . 323 | 288/323 (89%) | e−166 |
| | SEQ ID NO 2260 - | 1 . . . 291 | 288/323 (89%) | |
| | Homo sapiens, 291 aa. | | | |
| | [WO200190304-A2, | | | |
| | 29 NOV. 2001] | | | |
| AAG74165 | Human colon cancer | 1 . . . 323 | 288/323 (89%) | e−166 |
| | antigen protein SEQ | 13 . . . 303 | 288/323 (89%) | |
| | ID NO: 4929 - Homo | | | |
| | sapiens, 303 aa. | | | |
| | [WO200122920-A2, | | | |
| | 5 APR. 2001] | | | |

In a BLAST search of public sequence datbases, the NOV8a protein was found to have homology to the proteins shown in the BLASTP data in Table 8D.

TABLE 8D
Public BLASTP Results for NOV8a

| | | NOV8a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9NVV0 | CDNA FLJ10493 fis, | 1 . . . 323 | 290/323 (89%) | e−167 |
| | clone NT2RP2000274 | 1 . . . 291 | 290/323 (89%) | |
| | (Hypothetical 32.5 | | | |
| | kDa protein) - Homo | | | |
| | sapiens (Human), | | | |
| | 291 aa. | | | |
| Q9DAV9 | 1600017F22Rik protein | 1 . . . 323 | 210/325 (64%) | e−119 |
| | (RIKEN cDNA | 1 . . . 292 | 243/325 (74%) | |
| | 1600017F22 gene) - Mus | | | |
| | musculus (Mouse), | | | |
| | 292 aa. | | | |
| Q9H6F2 | CDNA: FLJ22328 fis, | 7 . . . 321 | 121/324 (37%) | 9e−59 |
| | clone HRC05632 | 11 . . . 297 | 191/324 (58%) | |
| | (Unknown) (Protein for | | | |
| | MGC: 3169) - Homo | | | |
| | sapiens (Human), | | | |
| | 299 aa. | | | |
| Q91WL2 | Similar to hypothetical | 7 . . . 321 | 117/323 (36%) | 5e−57 |
| | protein MGC3169 | 11 . . . 296 | 187/323 (57%) | |
| | (Hypothetical 33.3 kDa | | | |
| | protein) - | | | |
| | Mus musculus | | | |
| | (Mouse), 298 aa. | | | |
| Q9VXG9 | CG4239 protein | 14 . . . 278 | 86/268 (32%) | 2e−33 |
| | (GH25683P) - | 15 . . . 249 | 134/268 (49%) | |
| | Drosophila | | | |
| | melanogaster | | | |
| | (Fruit fly), 276 aa. | | | |

PFam analysis predicts that the NOV8a protein contains the domains shown in the Table 8E.

TABLE 8E
Domain Analysis of NOV8a

| | | | |
|---|---|---|---|
| | | Identities/ | |
| | | Similarities for | |
| Pfam | NOV8a Match | the Matched | Expect |
| Domain | Region | Region | Value |

No Significant Matches Found

### Example 9

The NOV9 clone was analyzed, and the nucleotide and encoded polypeptide sequences are shown in Table 9A.

TABLE 9A
NOV9 Sequence Analysis

| | |
|---|---|
| SEQ ID NO: 27 | 823 bp |

| | |
|---|---|
| NOV9a, | GAATCGCCCTTCTGCCAGCTTAGTGGAAGCTCTGCTCTGGGTGGAGAGCAGCCTCGCT |
| CG153403-01 DNA | |
| Sequence | TTGGTGACGCACAGTGCTGGGACCCTCCAGGAGCCCCGGGATTGAAGGATGGTGGCGG |
| | CCGTCCTGCTGGGGCTGAGCTGGCTCTGCTCTCCCCTGGGAGCTCTGGTCCTGGACTT |
| | CAACAACATCAGGAGCTCTGCTGACCTGCATGGGGCCCGGAAGGGCTCACAGTGCCTG |
| | TCTGACACGGACTGCAATACCAGAAAGTTCTGCCTCCAGCCCCGCGATGAGAAGCCGT |
| | TCTGTGCTACATGTCGTGGGTTGCGGAGGAGGTGCCAGCGAGATGCCATGTGCTGCCC |
| | CGGGACACTCTGTGTGAACGATGTTTGTACTACGATGGAAGATGCAACCCCAATATTA |
| | GAAAGGCAGCTTGATGAGCAAGATGGCACACATGCAGAAGGAACAACTGGGCACCCAG |
| | TCCAGGAAAGCCAACTCAAAAGGAAGCCAAGTATTAAGAAATCACAAGGCAGGAAGGG |
| | ACAAGAGGGAGAAAGTTGTCTGAGAACTTTTCACTGTGGCCCTGGACTTTGCTGTGCT |
| | CGTCATTTTTGGACGAAAATTTGTAAGCCAGTCCTTTTGGAGGGACAGGTCTGCTCCA |
| | GAAGAGGGCATAAAGACACTGCTCAAGCTCCAGAAATCTTCCAGCGTTGCGACTGTGG |
| | CCCTGGACTACTGTGTCGAAGCCAATTGACCAGCAATCGGCAGCATGCTCGATTAAGA |
| | GTATGCCAAAAAATAGAAAAGCTATAGATATTTCAAAATAAAGAAGAATCCACATCCA |
| | AAGGCGATTCA |

| | |
|---|---|
| ORF Start: ATG at 107 | ORF Stop: TAG at 779 |

| | | |
|---|---|---|
| SEQ ID NO: 28 | 224 aa | MW at 24864.3 kD |

| | |
|---|---|
| NOV9a, | MVAAVLLGLSWLCSPLGALVLDFNNIRSSADLHGARKGSQCLSDTDCNTRKFCLQPRD |
| CG153403-01 | |
| Protein Sequence | EKPFCATCRGLRRRCQRDANCCPGTLCVNDVCTTMEDATPILERQLDEQDGTHAEGTT |
| | GHPVQESQLKRKPSIKKSQGRKGQEGESCLRTFDCGPGLCCARHFWTKICKPVLLEGQ |
| | VCSRRGHKDTAQAPEIFQRCDCGPGLLCRSQLTSNRQHARLRVCQKIEKL |

| | |
|---|---|
| SEQ ID NO: 29 | 630 bp |

| | |
|---|---|
| NOV9b, | TGGAGAGCAGCCTCGCTTTGGTGACGCACAGTGCTGGGACCCTCCAGGAGCCCCGGGA |
| CG153403-02 DNA | |
| Sequence | ATTGAAGGATGGTGGCGGCCGTCCTGCTGGGGCTGAGCTGGCTCTGCTCTCCCCTGGG |
| | AGCTCTGGTCCTGGACTTCAACAACATCAGGAGCTCTGCTGACCTGCATGGGGCCCGG |
| | AAGGGCTCACAGTGCCTGTCTGACACGGACTGCAATACCAGAAAGTTCTGCCTCCAGC |
| | CCCGCGATGAGAAGCCGTTCTGTGCTACATGTCGTGGGTTGCGGAGGAGGTGCCAGCG |
| | AGACGCCATGTGCTGCCCTGGGACACTCTGTGTGAACGGACAAGAGGGAGAAAGTTGT |
| | CTGAGAACTTTTGACTGTGGCCCTGGACTTTGCTGTGCTCGTCATTTTTGGACGAAAA |
| | TTTGTAAGCCAGTCCTTTTGGAGGGACAGGTCTGCTCCAGAAGAGGGCATAAAGACAC |
| | TGCTCAAGCTCCAGAAATCTTCCAGCGTTGCGACTGTGGCCCTGGACTACTGTGTCGA |
| | AGCCAATTGGCCAGCAATCGGCAGCATGCTCGATTAAGAGTATGCCAAAAAATAGAAA |
| | AGCTATAAATATTTCAAAATAAAGAAGATCCACATGCAAAGGCGATTCCA |

| | |
|---|---|
| ORF Start: ATG at 67 | ORF Stop: TAA at 586 |

| | | |
|---|---|---|
| SEQ ID NO: 30 | 173 aa | MW at 19176.1 kD |

| | |
|---|---|
| NOV9b, | MVAAVLLGLSWLCSPLGALVLDFNNIRSSADLHGARKGSQCLSDTDCNTRKFCLQPRD |
| CG153403-02 | |
| Protein Sequence | EKPFCATCRGLRRRCQRDAMCCPGTLCVNGQEGESCLRTFDCGPGLCCARHFWTKICK |
| | PVLLEGQVCSRRGHKDTAQAPEIFQRCDCGPGLLCRSQLASNRQHARLRVCQKIEKL |

| | |
|---|---|
| SEQ ID NO: 31 | 484 bp |

| | |
|---|---|
| NOV9c, | CACCGGATCCCTGCTCCTGGACTTCAACAACATCAGGAGCTCTGCTGACCTGCATGGG |
| 305037558 DNA | |
| Sequence | GCCCGGAAGGGCTCACAGTGCCTGTCTGACACGGACTGCAATACCAGAAAGTTCTGCC |
| | TCCAGCCCCGCGATGAGAAGCCGTTCTGTGCTACATGTCGTGGGTTGCGGAGGAGGTG |
| | CCAGCGAGACGCCATGTGCTGCCCTGGGACACTCTGTGTGAACGGACAAGAGGGAGAA |
| | AGTTGTCTGAGAACTTTTGACTGTGGCCCTGGACTTTGCTGTGCTCGTCATTTTTGGA |
| | CGAAAATTTGTAAGCCAGTCCTTTTGGAGGGACAGGTCTGCTCCAGAAGAGGGCATAA |
| | AGACACTGCTCAAGCTCCAGAAATCTTCCAGCGTTGCGACTGTGGCCCTGGACTACTG |
| | TGTCGAAGCCAATTGGCCAGCAATCGGCAGCATGCTCGATTAAGAGTATGCCAAAAAA |
| | TAGAAAAGCTACTCGAGGGC |

| | |
|---|---|
| ORF Start: at 2 | ORF Stop: end of sequence |

| | | |
|---|---|---|
| SEQ ID NO: 32 | 161 aa | MW at 17937.4 kD |

| | |
|---|---|
| NOV9c, | TGSLVLDFNNIRSSADLHGARKGSQCLSDTDCNTRKFCLQPRDEKPFCATCRGLRRRC |
| 305037558 | |
| Protein Sequence | QRDAMCCPGTLCVNGQEGESCLRTEDCGPGLCCARHFWTKICKPVLLECQVCSRRGHK |
| | DTAQAPEIEQRCDCGPGLLCRSQLASNRQHARLRVCQKIEKLLEG |

| | |
|---|---|
| SEQ ID NO: 33 | 541 bp |

| | |
|---|---|
| NOV9d, | CACCGGATCCACCATGGTGGCGGCCGTCCTGCTGGGGCTGAGCTGGCTCTGCTCTCCC |
| 305037512 DNA | |
| Sequence | CTGGGAGCTCTGGTCCTGGACTTCAACAACATCAGGAGCTCTGCTGACCTGCATGGGG |
| | CCCGGAAGGGCTCACAGTGCCTGTCTGACACGGACTGCAATACCAGAAAGTTCTGCCT |
| | CCAGCCCCGCGATGAGAAGCCGTTCTGTGCTACATGTCGTGGGTTGCGGAGGAGGTGC |
| | CAGCGAGACGCCATGTGCTGCCCTGGGACACTCTGTGTGAACGGACAAGAGGGAGAAA |
| | GTTGTCTGAGAACTTTTGACTGTGGCCCTGGACTTTGCTGTGCTCGTCATTTTTGGAC |
| | GAAAATTTGTAAGCCAGTCCTTTTGGAGGGACAGGTCTGCTCCAGAAGAGGGCATAAA |
| | GACACTGCTCAAGCTCCAGAAATCTTCCAGCGTTGCGACTGTGGCCCTGGACTACTGT |
| | GTCGAAGCCAATTGGCCAGCAATCGGCAGCATGCTCGATTAAGAGTATGCCAAAAAAT |
| | AGAAAAGCTACTCGAGGGC |

| | |
|---|---|
| ORF Start: at 2 | ORF Stop: end of sequence |

| | | |
|---|---|---|
| SEQ ID NO: 34 | 180 aa | MW at 19821.7 kD |

| | |
|---|---|
| NOV9d, | TGSTMVAAVLLGLSWLCSPLGALVLDFNNIRSSADLHGARKGSQCLSDTDCNTRKFCL |
| 305037512 | |
| Protein Sequence | QPRDEKPFCATCRGLRRRCQRDAMCCPGTLCVNGQEGESCLRTFDCGPGLCCARHFWT |
| | KICKPVLLEGQVCSRRGHKDTAQAPEIFQRCDCGPGLLCRSQLASNRQHARLRVCQKI |
| | EKLLEG |

Sequence comparison of the above protein sequences yields the following sequence relationships shown in Table 9B.

TABLE 9B
Comparison of NOV9a against NOV9b through NOV9d.

| | | Identities/ |
|---|---|---|
| | | Similarities for |
| Protein | NOV9a Residues/ | the Matched |
| Sequence | Match Residues | Region |
| NOV9b | 1 . . . 224 | 172/224 (76%) |
| | 1 . . . 173 | 172/224 (76%) |
| NOV9c | 17 . . . 224 | 155/208 (74%) |
| | 2 . . . 158 | 156/208 (74%) |
| NOV9d | 1 . . . 224 | 172/224 (76%) |
| | 5 . . . 177 | 172/224 (76%) |

Further analysis of the NOV9a protein yielded the following properties shown in Table 9C.

TABLE 9C
Protein Sequence Properties NOV9a

| | |
|---|---|
| PSort | 0.7284 probability located in outside; 0.1000 |
| analysis: | probability located in endoplasmic reticulum |
| | (membrane); 0.1000 probability located in |
| | endoplasmic reticulum (lumen); 0.1000 |
| | probability located in microbody (peroxisome) |
| SignalP | Cleavage site between residues 19 and 20 |
| analysis: | |

A search of the NOV9a protein against the Geneseq database, a proprietary database that contains sequences published in patents and patent publication, yielded several homologous proteins shown in Table 9D.

TABLE 9D
Geneseq Results for NOV9a

| | | NOV9a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAY92075 | Human DKR-4 - | 1 . . . 224 | 222/224 (99%) | e−135 |
| | Homo sapiens, 224 aa. | 1 . . . 224 | 223/224 (99%) | |
| | [WO200018914-A2, | | | |
| | 06 APR. 2000] | | | |
| AAB08875 | Amino acid sequence | 1 . . . 224 | 222/224 (99%) | e−135 |
| | of a human Dickkopf | 1 . . . 224 | 223/224 (99%) | |
| | (Dkk)-4 protein - | | | |
| | Homo sapiens, 224 aa. | | | |
| | [WO200052047-A2, | | | |
| | 08 SEP. 2000] | | | |
| AAW73017 | Human cysteine-rich | 1 . . . 224 | 222/224 (99%) | e−135 |
| | secreted protein | 1 . . . 224 | 223/224 (99%) | |
| | CRSP-2 - Homo sapiens, | | | |
| | 224 aa. [WO9846755-A1, | | | |
| | 22 OCT. 1998] | | | |
| AAB66109 | Protein of the | 34 . . . 221 | 84/199 (42%) | 2e−37 |
| | invention #21 - | 65 . . . 259 | 109/199 (54%) | |
| | Unidentified, 259 aa. | | | |
| | [WO200078961-A1, | | | |
| | 28 DEC. 2000] | | | |
| AAU29148 | Human PRO polypeptide | 34 . . . 221 | 84/199 (42%) | 2e−37 |
| | sequence #125 - | 65 . . . 259 | 109/199 (54%) | |
| | Homo sapiens, 259 aa. | | | |
| | [WO200168848-A2, | | | |
| | 20 SEP. 2001] | | | |

In a BLAST search of public sequence datbases, the NOV9a protein was found to have homology to the proteins shown in the BLASTP data in Table 9E.

TABLE 9E
Public BLASTP Results for NOV9a

| | | NOV9a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9UBT3 | Dickkopf related | 1 . . . 224 | 222/224 (99%) | e−135 |
| | protein-4 precursor | 1 . . . 224 | 223/224 (99%) | |
| | (Dkk-4) (Dickkopf-4) | | | |
| | (hDkk-4) - Homo sapiens | | | |
| | (Human), 224 aa. | | | |
| Q8VEJ3 | Similar to dickkopf | 1 . . . 221 | 166/221 (75%) | e−101 |
| | (Xenopus laevis) | 1 . . . 221 | 185/221 (83%) | |
| | homolog 4 - Mus musculus | | | |
| | (Mouse), 221 aa. | | | |
| Q9UBU2 | Dickkopf related | 34 . . . 221 | 84/199 (42%) | 7e−37 |
| | protein-2 precursor | 65 . . . 259 | 109/199 (54%) | |
| | (Dkk-2) (Dickkopf-2) | | | |
| | (hDkk-2) - Homo sapiens | | | |
| | (Human), 259 aa. | | | |
| Q9QYZ8 | Dickkopf related | 34 . . . 221 | 85/200 (42%) | 9e−37 |
| | protein-2 precursor | 65 . . . 259 | 109/200 (54%) | |
| | (Dkk-2) (Dickkopf-2) | | | |
| | (mDkk-2) - Mus musculus | | | |
| | (Mouse), 259 aa. | | | |
| Q9PWH3 | Dickkopf1 - | 41 . . . 220 | 84/184 (45%) | 1e−36 |
| | Brachydanio | 68 . . . 239 | 105/184 (56%) | |
| | rerio (Zebrafish) | | | |
| | (Zebra danio), 240 aa. | | | |

PFam analysis predicts that the NOV9a protein contains the domains shown in the Table 9F.

TABLE 9F
Domain Analysis of NOV9a

| | | | |
|---|---|---|---|
| | | Identities/ | |
| | | Similarities for | |
| Pfam | NOV9a Match | the Matched | Expect |
| Domain | Region | Region | Value |

No Significant Matches Found

### Example 10

The NOV10 clone was analyzed, and the nucleotide and encoded polypeptide sequences are shown in Table 10A.

TABLE 10A
NOV10 Sequence Analysis

| | |
|---|---|
| SEQ ID NO: 35 | 878 bp |

| | |
|---|---|
| NOV10a, | ATGCCGCGCTTGTCTCTGCTCTTGCCGCTGCTGCTTCTGCTGCTGCTGCCGCTGCTGC |
| CG153424-01 DNA | |
| Sequence | CGCCGCTGTCCCCGAGCCTTGGGATCCGCGACGTGCGCGGCCGGCGCCCCAAGTGTGG |
| | TCCGTGCCGGCCAGAGGGCTGCCCGGCGCCTGCGCCCTGCCCGGCGCCCGGGATCTCG |
| | GCGCTCGACGAGTCCGGCTGCTGCGCCCGCTGCCTGGGAGCCGAGGGCGCGAGCTGCG |
| | GGGGCCGCGCCGGCGGGCGCTGTGGCCCCGGCCTGGTATGCGCGAGCCAGGCCGCTGG |
| | GGCAGCGCCCGACGGCACCGGGCTCTGCGTGTGCGCGCAGCGCGGCACCGTCTGCGGC |
| | TCCGACGGTCGCTCGTACCCCAGCGTCTGCGCGCTGCGCCTGCCCGCTCGGCACACGC |
| | CCCGCGCGCACCCCGGTCACCTGCACAAGGCGCGCGACGGCCCTTGCGAGTTCGCTCC |
| | TGTGGTCGTCGTTCCTCCCCGAAGTGTTCACAACGTCACCGGGGCGCAGGTGGGCCTC |
| | TCCTGTGAAGTGAGGGCTGTGCCTACCCCAGTCATCACGTGGAGAAAGGTAACGAAGT |
| | CCCCTGAGGGCACCCAAGCACTGGAGGAGCTGCCTGGGGACCATGTCAATATAGCTGT |
| | CCAAGTGCGAGGGGGCCCTTCTGACCATGAGGCCACCGCCTGGATTTTGATCAACCCC |
| | CTGCGAAAGGAGGATGAGGGTGTGTACCAGTGCCATGCAGCCAACATGGTGGGAGAGG |
| | CTGAGTCCCACAGCACAGTGACGGTTCTAGATCTGAGTAAATACAGGAGCTTCCACTT |
| | CCCAGCTCCCGATGACCGCATGTGATGGAGAAATGTACATGTTCTAAGTCATTTTCAG |
| | TATTTTAC |

| | |
|---|---|
| ORF Start: ATG at 1 | ORF Stop: TGA at 835 |

| | | |
|---|---|---|
| SEQ ID NO: 36 | 278 aa | MW at 29005.1 kD |

| | |
|---|---|
| NOV10a, | MPRLSLLLPLLLLLLLPLLPPLSPSLGIRDVGGRRPKCGPCRPEGCPAPAPCPAPGIS |
| CG153424-01 | |
| Protein Sequence | ALDECGCCARCLGAEGASCGGRAGGRCGPGLVCASQAAGAAPEGTGLCVCAQRGTVCG |
| | SDGRSYPSVCALRLRARHTPRAHPGHLHKARDGPCEFAPVVVVPPRSVHNVTGAQVGL |
| | SCEVRAVPTPVITWRKVTKSPEGTQALEELPGDHVNIAVQVRGGPSDHEATAWILIMP |
| | LRKEDEGVYQCHAANMVGEAESHSTVTVLDLSKYRSFHFPAPDDRM |

Further analysis of the NOV10a protein yielded the following properties shown in Table 10B.

TABLE 10B
Protein Sequence Properties NOV10a

| | |
|---|---|
| PSort | 0.8200 probability located in endoplasmic |
| analysis: | reticulum (membrane); 0.1900 probability |
| | located in plasma membrane; 0.1000 probability |
| | located in endoplasmic reticulum (lumen); |
| | 0.1000 probability located in outside |
| SignalP | Cleavage site between residues 28 and 29 |
| analysis: | |

A search of the NOV 10a protein against the Geneseq database, a proprietary database that contains sequences published in patents and patent publication, yielded several homologous proteins shown in Table 10C.

TABLE 10C
Geneseq Results for NOV10a

| | | NOV10a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAU08753 | Human insulin-like | 1 . . . 278 | 278/278 (100%) | e−169 |
| | growth factor binding | 1 . . . 278 | 278/278 (100%) | |
| | protein-like | | | |
| | polypeptide #3 - | | | |
| | Homo sapiens, 278 aa. | | | |
| | [WO200175064-A2, | | | |
| | 11 OCT. 2001] | | | |
| AAE15654 | Human growth factor | 1 . . . 278 | 276/282 (97%) | e−164 |
| | binding protein-like | 1 . . . 282 | 276/282 (97%) | |
| | protein, NOV5 - | | | |
| | Homo sapiens, 282 aa. | | | |
| | [WO200194416-A2, | | | |
| | 13 DEC. 2001] | | | |
| AAU08755 | Human insulin-like | 1 . . . 156 | 154/156 (98%) | 4e−93 |
| | growth factor binding | 1 . . . 156 | 155/156 (98%) | |
| | protein-like | | | |
| | polypeptide #2 - | | | |
| | Homo sapiens, 390 aa. | | | |
| | [WO200175064-A2, | | | |
| | 11 OCT. 2001] | | | |
| ABG01683 | Novel human diagnostic | 1 . . . 156 | 154/156 (98%) | 4e−93 |
| | protein #1674 - | 1 . . . 156 | 155/156 (98%) | |
| | Homo sapiens, 390 aa. | | | |
| | [WO200175067-A2, | | | |
| | 11 OCT. 2001] | | | |
| AAR79102 | Prostaglandin I2 (PGI2) | 11 . . . 262 | 115/263 (43%) | 4e−59 |
| | prodn. promoter - Homo | 16 . . . 267 | 141/263 (52%) | |
| | sapiens, 282 aa. | | | |
| | [WO9429448-A, | | | |
| | 22 DEC. 1994] | | | |

In a BLAST search of public sequence datbases, the NOV10a protein was found to have homology to the proteins shown in the BLASTP data in Table 10D.

TABLE 10D
Public BLASTP Results for NOV10a

| | | NOV10a | Identities/ | |
|---|---|---|---|---|
| Protein | | Match | for the | Expect |
| Number | Protein/Organism/Length | Residues | Matched Portion | Value |
| Q8WX77 | BA113O24.1 (similar | 1 . . . 278 | 278/278 (100%) | e−169 |
| | to insulin-like growth | 1 . . . 278 | 278/278 (100%) | |
| | factor binding protein) - | | | |
| | Homo sapiens | | | |
| | (Human), 278 aa. | | | |
| BAA21725 | IGFBP-LIKE PROTEIN - | 1 . . . 276 | 212/276 (76%) | e−128 |
| | Mus musculus | 1 . . . 268 | 234/276 (83%) | |
| | (Mouse), 270 aa. | | | |
| Q07822 | MAC25 protein - | 11 . . . 262 | 115/263 (43%) | 1e−58 |
| | Homo sapiens | 16 . . . 267 | 141/263 (52%) | |
| | (Human), 277 aa. | | | |
| Q16270 | Insulin-like growth factor | 11 . . . 262 | 115/263 (43%) | 1e−58 |
| | binding protein 7 | 16 . . . 267 | 141/263 (52%) | |
| | precursor (IGFBP-7) | | | |
| | (IBP- 7) (IGF-binding | | | |
| | protein 7) (MAC25 protein) | | | |
| | (Prostacyclin-stimulating | | | |
| | factor) (PGI2-stimulating | | | |
| | factor) - Homo sapiens | | | |
| | (Human), 282 aa. | | | |
| Q61581 | Mac25 protein - Mus | 11 . . . 262 | 114/263 (43%) | 5e−57 |
| | musculus (Mouse), 281 aa. | 15 . . . 266 | 140/263 (52%) | |

PFam analysis predicts that the NOV 10a protein contains the domains shown in the Table 10E.

TABLE 10E
Domain Analysis of NOV10a

| | | Identities/ | |
|---|---|---|---|
| | | Similarities for | |
| Pfam | NOV10a Match | the Matched | Expect |
| Domain | Region | Region | Value |
| kazal | 91 . . . 151 | 18/63 (29%) | 7.5e−05 |
| | | 45/63 (71%) | |
| ig | 169 . . . 245 | 16/80 (20%) | 5.4e−08 |
| | | 59/80 (74%) | |

### Example 11

The NOV11 clone was analyzed, and the nucleotide and encoded polypeptide sequences are shown in Table 11A.

TABLE 11A
NOV11 Sequence Analysis

| | |
|---|---|
| SEQ ID NO: 37 | 1245 bp |

| NOV11a, | CAGGAACGGGCTCCGCGGACGACGCGCTCCAGGCACGCACAGGCAGCGGGCCTCCCAC | |
|---|---|---|
| CG157567-01 DNA | | |
| Sequence | CGCGCGTGCCGGGGGCGGGGGGGCTGCCCCCATGCGGGGCCCTTCCTGGTTGCGGCCT | |
| | CGGCCGCTGCTGCTGCTGTTGCTCCTGCTGTCGCCTTGGCCTGTCTGGGCCCATGTGT | |
| | CGGCCACGGCCTCGCCCTCGGGGTCCCTGGGCGCCCCGGACTGCCCCGAGGTGTGCAC | |
| | GTGCGTGCCGGGAGGCCTGGCCAGCTGCTCGGCACTCTCGCTGCCCGCCGTGCCCCCG | |
| | GGCCTGAGCCTGCGCCTGCGCGCGCTGCTGCTOGACCACAACCGCGTCCGTGCGCTGC | |
| | CGCCAGGTGCCTTCGCGGGAGCGGGCGCGCTACAGCGCCTGGACCTGCGCGAGAGCGG | |
| | GCTGCACTCGGTGCATGTGCGAGCCTTCTGGGGCCTGGGCGCGCTGCAGCTGCTGGAC | |
| | CTGAGCGCCAACCAGCTGGAAGCACTGGCACCAGGGACTTTCGCGCCGCTGCGCGCGC | |
| | TGCGCAACCTCTCATTGGCCGGCAACCGGCTGGCGCGCCTGGAGCCCGCGGCGCTAGG | |
| | CGCGCTCCCGCTGCTGCGCTCACTCAGCCTGCAGGACAACGAGCTGGCGGCACTCGCG | |
| | CCGGGGCTGCTGGCCCGCCTGCCCGCTCTAOACGCGCTGCACCTGCGCGGCGACCCTT | |
| | GGGGCTGCGGCTGCGCGCTGCGCCCGCTCTGCGCCTGGCTGCCCCGGCACCCGCTCCC | |
| | CGCGTCAGAGCCCGAGACGGTGCTCTGCGTGTGGCCGGGACGCCTGACGCTCAGCCCC | |
| | CTGACTGCCTTTTCCGACGCCGCCTTTAGCCATTGCGCGCAGCCGCTCGCCCTGCGGC | |
| | ACCTGOCCGTGGTTTACACGCTCGGGCCGGCCTCCTTCCTCGTCAGCCTGGCTTCCTG | |
| | CCTGGCGCTGGGCTCTGGGCTCACCGCCTGCCGTGCGCGCCGCCGCCGCCTCCGCACC | |
| | CCCGCCCTCCGCCCGCCGAGACCGCCAGACCCGAACCCCGATCCCGACCCCCACGGCT | |
| | GTGCCTCGCCCCCGGACCCGGGGAGCCCCGCCGCTGCCGCCCAAGCCTGAGCGGCCGC | |
| | GGCCGCCTGGAGCGCTCGAAGCTTCCCCCATGCCTTTGCCCTCCCTTTACACTGTCTG | |
| | CCGGCGTCAACAAGCGACACAGACCGAAAAAAAAAAAAAAAAAAAAAAAAAAAAAAAA | |
| | AAAAAAAAAAAAAAAAAACAAAAAATT | |
| | ORF Start: ATG at 90 | ORF Stop: TGA at 1092 |

| | | |
|---|---|---|
| SEQ ID NO: 38 | 334 aa | MW at 34891.0kD |

| | |
|---|---|
| NOV11a, | MRGPSWLRPRPLLLLLLLLSPWPVWAHVSATASPSGSLGAPDCPEVCTCVPGGLASCS |
| CG157567-01 | |
| Protein Sequence | ALSLPAVPPGLSLRLRALLLDHNRVRALPPGAFAGAGALQRLDLRENGLHSVHVRAFW |
| | GLGALQLLDLSANQLEALAPGTFAPLRALRNLSLAGNRLARLEPAALGALPLLRSLSL |
| | QDNELAALAPGLLGRLPALDALHLRGNPWGCGCALRPLCAWLRRHPLPASEAETVLCV |
| | WPGRLTLSPLTAFSDAAFSHCAQPLALRDLAVVYTLGPASFLVSLASCLALGSGLTAC |
| | RARRRRLRTAALRPPRPPDPNPDPDPHGCASPADPGSPAAAAQA |

Further analysis of the NOV11a protein yielded the following properties shown in Table 11B.

TABLE 11B
Protein Sequence Properties NOV11a

| | |
|---|---|
| PSort | 0.5947 probability located in outside; 0.1000 |
| analysis: | probability located in endoplasmic reticulum |
| | (membrane); 0.1000 probability located in |
| | endoplasmic reticulum (lumen); 0.1000 |
| | probability located in microbody (peroxisome) |
| SignalP | Cleavage site between residues 27 and 28 |
| analysis: | |

A search of the NOV11a protein against the Geneseq database, a proprietary database that contains sequences published in patents and patent publication, yielded several homologous proteins shown in Table 11C.

TABLE 11C
Geneseq Results for NOV11a

| | | NOV11a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAY41496 | Fragment of human secreted | 40 . . . 334 | 235/303 (77%) | e−120 |
| | protein encoded by gene 70 - | 77 . . . 368 | 240/303 (78%) | |
| | Homo sapiens, 368 aa. | | | |
| | [WO9947540-A1, 23 SEP. 1999] | | | |
| AAB07469 | A human leucine-rich repeat | 9 . . . 290 | 93/284 (32%) | 2e−28 |
| | protein designated Zlrr3 - | 14 . . . 286 | 126/284 (43%) | |
| | Homo sapiens, 298 aa. | | | |
| | [WO200042184-A1, 20 JUL. 2000] | | | |
| AAU12198 | Human PRO1341 polypeptide | 43 . . . 290 | 85/250 (34%) | 9e−28 |
| | sequence - Homo sapiens, 281 | 21 . . . 269 | 116/250 (46%) | |
| | aa. [WO200140466-A2, 07 JUN. | | | |
| | 2001] | | | |
| AAW96707 | Protein sequence of the | 34 . . . 237 | 73/204 (35%) | 8e−27 |
| | specification - Homo sapiens, | 273 . . . 472 | 107/204 (51%) | |
| | 1534 aa. [JP11018777-A, 26 | | | |
| | JAN. 1999] | | | |
| AAW96706 | Protein sequence of the | 34 . . . 237 | 73/204 (35%) | 8e−27 |
| | specification - Homo sapiens, | 247 . . . 446 | 107/204 (51%) | |
| | 1508 aa. [JP11018777-A, 26 | | | |
| | JAN. 1999] | | | |

In a BLAST search of public sequence datbases, the NOV11a protein was found to have homology to the proteins shown in the BLASTP data in Table 11D.

TABLE 11D
Public BLASTP Results for NOV11a

| | | NOV11a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q91W20 | Unknown (Protein for | 1 . . . 332 | 219/332 (65%) | e−112 |
| | MGC: 6965) (Hypothetical 35.7 | 1 . . . 328 | 235/332 (69%) | |
| | kDa protein) - Mus musculus | | | |
| | (Mouse), 331 aa. | | | |
| Q96B32 | Hypothetical 35.0 kDa | 62 . . . 285 | 81/226 (35%) | 6e−27 |
| | protein - Homo sapiens | 70 . . . 294 | 108/226 (46%) | |
| | (Human), 317 aa (fragment). | | | |
| BAA32465 | MEGF4 - Homo sapiens | 34 . . . 237 | 73/204 (35%) | 2e−26 |
| | (Human), 1618 aa (fragment). | 357 . . . 556 | 107/204 (51%) | |
| O75093 | Slit-1 protein - Homo | 34 . . . 237 | 73/204 (35%) | 2e−26 |
| | sapiens (Human), 1534 aa. | 273 . . . 472 | 107/204 (51%) | |
| Q9WVB5 | SLIT1 - Mus musculus | 30 . . . 237 | 72/208 (34%) | 4e−26 |
| | (Mouse), 1531 aa. | 269 . . . 472 | 109/208 (51%) | |

PFam analysis predicts that the NOV11a protein contains the domains shown in the Table 11E.

TABLE 11E
Domain Analysis of NOV11a

| | | Identities/ | |
|---|---|---|---|
| | | Similarities for | |
| Pfam | NOV11a Match | the Matched | Expect |
| Domain | Region | Region | Value |
| LRRNT | 42 . . . 70 | 13/31 (42%) | 0.86 |
| | | 20/31 (65%) | |
| LRR | 96 . . . 119 | 9/25 (36%) | 0.52 |
| | | 16/25 (64%) | |
| LRR | 120 . . . 143 | 11/25 (44%) | 0.043 |
| | | 18/25 (72%) | |
| LRR | 144 . . . 167 | 10/25 (40%) | 0.33 |
| | | 17/25 (68%) | |
| LRRCT | 201 . . . 254 | 18/55 (33%) | 0.0078 |
| | | 30/55 (55%) | |

### Example 12

The NOV12 clone was analyzed, and the nucleotide and encoded polypeptide sequences are shown in Table 12A.

TABLE 12A
NOV12 Sequence Analysis

| | |
|---|---|
| SEQ ID NO:39 | 838 bp |

| NOV12a, | TCAAAGGAAACTGACAAATTATCCCCACCTGCCAGAAGAAGAAATCCTCACTGGACGG | |
|---|---|---|
| CG157760-01 DNA | | |
| Sequence | CTTCCTGTTTCCTGTGGTTCATTATCTGATTGGCTGCAGGGATGAAAGTTTTTAAGTT | |
| | CATAGGACTGATGATCCTCCTCACCTCTGCGCTTTCAGCCGGTTCAGGACAAAGTCCA | |
| | ATCACTGTGCTGTGCTCCATAGACTGGTTCATGGTCACAGTGCACCCCTTCATGCTAA | |
| | ACAACGATGTGTGTGTACACTTTCATGAACTACACTTGGGCCTGGGTTGCCCCCCAAA | |
| | CCATGTTCAGCCACACGCCTACCAGTTCACCTACCGTGTTACTGAATGTGGCATCAGG | |
| | GCCAAAGCTGTCTCTCAGGACATGGTTATCTACAGCACTGAGATACACTACTCTTCTA | |
| | AGGGCACGCCATCTAAGTTTGTGATCCCAGTGTCATGTGCTGCCCCCCAAAAGTCCCC | |
| | ATGGCTCACCAAGCCCTGCTCCATGAGAGTAGCCAGCAAGAGCAGGGCCACAGCCCAG | |
| | AAGGATGAGAAATGCTACGAGGTGTTCAGCTTGTCACAGTCCAGTCAAAGGCCCAACT | |
| | GCGATTGTCCACCTTGTGTCTTCAGTGAAGAAGACCATACCCAGGTCCCTTGTCACCA | |
| | AGCAGGGGCTCAGGAGGCTCAACCTCTGCAGCCATCTCACTTTCTTGATATTTCTGAG | |
| | GATTGGTCTCTTCACACAGATGATATGATTGGGTCCATGTGATCCTCAGGTTTGOGGT | |
| | CTCCTGAAGATGCTATTTCTAGAATTAGTATATAGTGTACAAATGTCTGACAAATAAG | |
| | TCCTCTTGTGACCCTCATTAAGGCCA | |
| | ORF Start: ATG at 100 | ORF Stop: TGA at 736 |

| | | |
|---|---|---|
| SEQ ID NO: 40 | 212 aa | MW at 23581.8kD |

| NOV12a, | MKVFKFIGLMILLTSALSAGSGQSPMTVLCSIDWFMVTVHPFMLNNDVCVHFHELHLG | |
|---|---|---|
| CG157760-01 | | |
| Protein Sequence | LGCPPNHVQPHAYQFTYRVTECGIRAKAVSQDMVIYSTEIHYSSKGTPSKFVIPVSCA | |
| | APQKSPWLTKPCSMRVASKSRATAQKDEKCYEVFSLSQSSQRPNCDCPPCVFSEEEHT | |
| | QVPCHQAGAQEAQPLQPSHFLDISEDWSLHTDDMIGSM | |
| | SEQ ID NO: 41 | 697 bp |

| NOV12b, | TCAAAGGAAACTOACAAATTATCCCCAGCTGCCAAAAGAAGAAATCCTCACTGGACGG | |
|---|---|---|
| CG157760-02 DNA | | |
| Sequence | CTTCCTGTTTCCTGTGGTTCATTATCTGATTGGCTGCAGGGATGAAAGTTTTTAAGTT | |
| | CATAGGACTGATGATCCTCCTCACCTCTGCGTTTTCAGCCGGTTCAGGACAAAGTCCA | |
| | ATGACTGTGCTGTGCTCCATAGACTGGTTCATGGTCACAGTGCACCCCTTCATGCTAA | |
| | ACAACGATGTGTGTGTACACTTTCATGAACTACACTTGGGCCTGGGTTCCCCCCCAAA | |
| | CCATGTTCAGCCACACGCCTACCAGTTCACCTACCGTGTTACTGAATGTGOCATCAGG | |
| | CCCAGCAAGAGCAGGGCCACAGCCCAGAAGGATGAGAAATGCTACGAGGTGTTCAGCT | |
| | TGTCACAGTCCAGTCAAAGGCCCAACTGCGATTGTCCACCTTGTGTCTTCAGTGAAGA | |
| | AGAGCATACCCAGGTCCCTTGTCACCAAGCAGGGGCTCAGGAGGCTCAACCTCTGCAG | |
| | CCATCTCACTTTCTTGATATTTCTGAGGATTGGTCTCTTCACACAGATGATATGATTG | |
| | GGTCCATGTGATCCTGAGGTTTGGGGTCTCCTGAAGATGCTATTTCTAGATTTAGTAT | |
| | ATAGTGTACAAATGTCTGACAAATAAGTGCTCTTGTGACCCTCATGTGAGGGCGATTC | |
| | C | |
| | ORF Start: ATG at 100 | ORF Stop: TGA at 589 |

| | | |
|---|---|---|
| SEQ ID NO: 42 | 163 aa | MW at 18277.GkD |

| | |
|---|---|
| NOV12b, | MKVFKFIGLMILLTSAFSAGSGQSPMTVLCSIDWFMVTVHPFMLNNDVCVHFHELHLG |
| CG157760-02 | |
| Protein Sequence | LGCPPNHVQPHAYQFTYRVTECGIRASKSRATAQKDEKCYEVFSLSQSSQRPNCDCPP |
| | CVFSEEEHTQVPCHQAGAQEAQPLQPSHFLDISEDWSLHTDDMIGSM |

Sequence comparison of the above protein sequences yields the following sequence relationships shown in Table 12B.

TABLE 12B
Comparison of NOV12a against NOV12b.

| | | Identities/ |
|---|---|---|
| | | Similarities for |
| Protein | NOV12a Residues/ | the Matched |
| Sequence | Match Residues | Region |
| NOV12b | 1 . . . 212 | 162/212 (76%) |
| | 1 . . . 163 | 162/212 (76%) |

Further analysis of the NOV12a protein yielded the following properties shown in

TABLE 12C
Protein Sequence Properties NOV12a

| | |
|---|---|
| PSort | 0.6568 probability located in outside; 0.1000 |
| analysis: | probability located in endoplasmic reticulum |
| | (membrane); 0.1000 probability located in |
| | endoplasmic reticulum (lumen); 0.1000 |
| | probability located in lysosome (lumen) |
| SignalP | Cleavage site between residues 23 and 24 |
| analysis: | |

A search of the NOV12a protein against the Geneseq database, a proprietary database that contains sequences published in patents and patent publication, yielded several homologous proteins shown in Table 12D.

TABLE 12D
Geneseq Results for NOV12a

| | | NOV12a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABP61861 | Human polypeptide SEQ ID NO | 1 . . . 212 | 211/212 (99%) | e−126 |
| | 215 - Homo sapiens, 212 aa. | 1 . . . 212 | 211/212 (99%) | |
| | [US2002065394-A1, 30 MAY | | | |
| | 2002] | | | |
| AAM93517 | Human polypeptide, SEQ ID NO: | 1 . . . 212 | 211/212 (99%) | e−126 |
| | 3243 - Homo sapiens, 212 | 1 . . . 212 | 211/212 (99%) | |
| | aa. [EP1130094-A2, 05 SEP. | | | |
| | 2001] | | | |
| AAY94302 | Human corticosteroid | 1 . . . 212 | 211/212 (99%) | e−126 |
| | synthesis-associated | 1 . . . 212 | 211/212 (99%) | |
| | protein - Homo sapiens, 212 aa. | | | |
| | [WO200028027-A2, 18 MAY | | | |
| | 2000] | | | |
| AAW73630 | Human secreted protein clone | 1 . . . 212 | 211/212 (99%) | e−126 |
| | ej265_4 - Homo sapiens, 212 | 1 . . . 212 | 211/212 (99%) | |
| | aa. [WO9855614-A2, 10 DEC. | | | |
| | 1998] | | | |
| AAY12939 | Amino acid sequence of a | 1 . . . 212 | 172/212 (81%) | 5e−96 |
| | human secreted peptide - | 1 . . . 212 | 179/212 (84%) | |
| | Homo sapiens, 213 aa. | | | |
| | [WO9911293-A1, 11 MAR. 1999] | | | |

In a BLAST search of public sequence datbases, the NOV12a protein was found to have homology to the proteins shown in the BLASTP data in Table 12E.

TABLE 12E
Public BLASTP Results for NOV12a

| | | NOV12a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9HBJ0 | PLAC1 (Placenta-specific 1) - | 1 . . . 212 | 211/212 (99%) | e−126 |
| | Homo sapiens (Human), 212 aa. | 1 . . . 212 | 211/212 (99%) | |
| Q9JI83 | EPCS26 (PLAC1) (Placental | 1 . . . 171 | 104/171 (60%) | 1e−60 |
| | specific protein 1) - Mus | 1 . . . 171 | 134/171 (77%) | |
| | musculus (Mouse), 173 aa. | | | |
| BAC04191 | CDNA FLJ36198 fis, clone | 9 . . . 125 | 38/118 (32%) | 7e−17 |
| | TESTI2028242, weakly similar | 5 . . . 122 | 70/118 (59%) | |
| | to Mus musculus EPCS26 mRNA - | | | |
| | Homo sapiens (Human), 158 aa. | | | |
| Q925U0 | Initiate factor 3 (Oocyte- | 7 . . . 122 | 34/117 (29%) | 6e−09 |
| | secreted protein 1 | 8 . . . 122 | 62/117 (52%) | |
| | precursor) - Mus musculus | | | |
| | (Mouse), 202 aa. | | | |
| BAC11848 | Initiate factor 3 2 - Mus | 7 . . . 88 | 25/83 (30%) | 3e−05 |
| | musculus (Mouse), 92 aa. | 8 . . . 89 | 46/83 (55%) | |

PFam analysis predicts that the NOV12a protein contains the domains shown in the Table 12F.

TABLE 12F
Domain Analysis of NOV12a

| | | | |
|---|---|---|---|
| | | Identities/ | |
| | | Similarities for | |
| Pfam | NOV12a Match | the Matched | Expect |
| Domain | Region | Region | Value |

No Significant Matches Found

### Example 13

The NOV13 clone was analyzed, and the nucleotide and encoded polypeptide sequences are shown in Table 13A.

TABLE 13A
NOV13 Sequence Analysis

| | |
|---|---|
| SEQ ID NO: 43 | 1103 bp |

| NOV13 a, | AAGCAGGCTGGTACGCCCTGGAGTTAANGGATGGCTGCGGGTTTGGCGGCGCTGCGCC | |
|---|---|---|
| CC157844-01 DNA | | |
| Sequence | GGCAGGCAGCGAGGCCGGGTCGGGCCCTGGGCCCTCGCGCCCCTCCCGCGAGGCCTGT | |
| | CATGCAGGGCCCCGCCGGGAACGCGAGCCGGGGACTGCCAGGCGGGCCGCCCTCCACA | |
| | GTCGCGTCCGGGGCGGGCCGCTGCGAGAGCGGCGCGCTCATGCACAGCTTCGGCATCT | |
| | TCCTGCACCGGCTGCTCGGCGTCGTGGCCTTCAGCACGTTAATGGTCAAACGCTTCAG | |
| | AGAACCAAAGCATGAAAGACGTCCGTGGAGGATATGGTTTTTAGACACTTCCAAACAA | |
| | GCCATAGGAATGCTGTTCATCCACTTTGCAAATGTATACCTAGCAGATCTCAGTGAAG | |
| | AGGACCCTTGTTCACTGTACCTCATCAACTTCCTCCTGGACGCCACTGTGGGCATGCT | |
| | GCTCATCTACGTGGGGGTGCGCGCCGTCAGCGTCCTGGTAGAGTGGCAGCAGTGGGAG | |
| | TCCCTGCGCTTCGGCGAATATGGAGACCCTCTGCAGTGTGGAGCCTGGGTCGGGCAGT | |
| | GCGCTCTTTACATCGTGATCATGATTTTTGAAAAGTCTGTCGTCTTCATCGTCCTCCT | |
| | CCTACTCCAGTGGAAAAAGGTGGCCCTATTGAATCCAATTGAAAACCCCGACCTGAAG | |
| | CAGGCCATCGTCATGCTGATCGTCCCCTTCTTTGTCAACGCTTTGATGTTTTGGGTAG | |
| | TGGACAATTTCCTCATGAGAAAGGGGAAGACGAAAGCTAAGCTAGAAGAAAGGGGAGC | |
| | CAACCAGGACTCGAGGAATGGGAGCAAGGTCCGCTACCGGAGGGCCGCATCCCACGAG | |
| | GAGTCTGAGTCTGAGATCCTGATCTCAGCGGATGATGAGATGGAGGAGTCCGACGTGG | |
| | AGGAGGACCTCCGCAGACTGACCCCCCTCAAGCCTGTGAAGAAAAAGAAGCACCGCTT | |
| | TGGGCTACCCGTATGACACATTCCCATGCTGGGGGTGACGGGACGGCCCCGCCAGCCG | |
| | CTGGTGTCCACAGGTCATCCCACAGCATCGTTCCTTACCCTCTCTCTGCCCTTCACCC | |
| | G | |
| | ORF Start: ATG at 31 | ORF Stop: TGA at 1000 |

| | | |
|---|---|---|
| SEQ ID NO: 44 | 323 aa | MW at 36089.9kD |

| | |
|---|---|
| NOV13a, | MAAGLAALRRQAARPGRALGPRAPPARPVMQGPAGNASRGLPGGPPSTVASGAGRCES |
| CG157844-01 | |
| Protein Sequence | GALMHSFGIFLQGLLGVVAFSTLMVKRFREPKHERRPWRIWFLDTSKQAIGMLFIHFA |
| | NVYLADLSEEDPCSLYLINFLLDATVGMLLIYVGVRAVSVLVEWQQWESLRFGEYGDP |
| | LQCGAWVGQCALYIVIMIFEKSVVFIVLLLLQWKKVALLNPIENPDLKLAIVMLIVPF |
| | FVNALMFWVVDNFLMRKGKTKAKLEERGANQDSRNGSKVRYRRAASHEESESEILISA |
| | DDEMEESDVEEDLRRLTPLKPVKKKKHRFGLPV |

Further analysis of the NOV13a protein yielded the following properties shown in Table 13B.

TABLE 13B
Protein Sequence Properties NOV13a

| | |
|---|---|
| PSort | 0.6113 probability located in mitochondrial |
| analysis: | inner membrane; 0.6000 probability located in |
| | plasma membrane; 0.4387 probability located in |
| | mitochondrial intermembrane space; |
| | 0.4000 probability located in Golgi body |
| SignalP | No Known Signal Sequence Predicted |
| analysis: | |

A search of the NOV13a protein against the Geneseq database, a proprietary database that contains sequences published in patents and patent publication, yielded several homologous proteins shown in Table 13C.

TABLE 13C
Geneseq Results for NOV13a

| | | NOV13a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB41574 | Human ORFX ORF1338 | 144 . . . 323 | 180/180 (100%) | e−100 |
| | polypeptide sequence SEQ ID | 1 . . . 180 | 180/180 (100%) | |
| | NO: 2676 - Homo sapiens, 180 | | | |
| | aa. [WO200058473-A2, 05 OCT. | | | |
| | 2000] | | | |
| ABG21481 | Novel human diagnostic | 233 . . . 306 | 52/74 (70%) | 3e−18 |
| | protein #21472 - Homo | 48 . . . 120 | 56/74 (75%) | |
| | sapiens, 507 aa. | | | |
| | [WO200175067-A2, 11 OCT. | | | |
| | 2001] | | | |
| AAG64212 | Murine HSP47 interacting | 11 . . . 53 | 23/52 (44%) | 0.21 |
| | protein, #2 - Mus sp, 255 | 65 . . . 115 | 27/52 (51%) | |
| | aa. [JP2001145493-A, 29 MAY | | | |
| | 2001] | | | |
| ABB53290 | Human polypeptide #30 - Homo | 11 . . . 53 | 23/52 (44%) | 0.27 |
| | sapiens, 255 aa. | 65 . . . 115 | 27/52 (51%) | |
| | [WO200181363-A1, 01 NOV. | | | |
| | 2001] | | | |
| ABG20114 | Novel human diagnostic | 7 . . . 61 | 22/55 (40%) | 0.35 |
| | protein #20105 - Homo | 441 . . . 494 | 26/55 (47%) | |
| | sapiens, 710 aa. | | | |
| | [WO200175067-A2, 11 OCT. | | | |
| | 2001] | | | |

In a BLAST search of public sequence datbases, the NOV13a protein was found to have homology to the proteins shown in the BLASTP data in Table 13D.

TABLE 13D
Public BLASTP Results for NOV13a

| | | NOV13a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9D7D4 | 2310014H19Rik protein - Mus | 30 . . . 323 | 277/294 (94%) | e−157 |
| | musculus (Mouse), 288 aa. | 1 . . . 288 | 280/294 (95%) | |
| Q9D8S1 | 1810038N08Rik protein - Mus | 30 . . . 323 | 277/294 (94%) | e−157 |
| | musculus (Mouse), 288 aa. | 1 . . . 288 | 280/294 (95%) | |
| Q8R3UO | Similar to RIKEN cDNA | 144 . . . 323 | 170/180 (94%) | 5e−91 |
| | 1810038N08 gene - Mus | 1 . . . 174 | 171/180 (94%) | |
| | musculus (Mouse), 174 aa. | | | |
| T49501 | hypothetical protein | 19 . . . 302 | 87/354 (24%) | 3e−17 |
| | B14D6.530 [imported] - | 149 . . . 496 | 148/354 (41%) | |
| | Neurospora crassa, 556 aa. | | | |
| Q12042 | P2558 protein (ORF | 49 . . . 246 | 63/227 (27%) | 3e−16 |
| | YPL162C) - Saccharomyces | 3 . . . 224 | 111/227 (48%) | |
| | cerevisiae (Baker's yeast), | | | |
| | 273 aa. | | | |

PFam analysis predicts that the NOV13a protein contains the domains shown in the Table 13E.

TABLE 13E
Domain Analysis of NOV13a

| | | | |
|---|---|---|---|
| | | Identities/ | |
| | | Similarities for | |
| Pfam | NOV13a Match | the Matched | Expect |
| Domain | Region | Region | Value |

No Significant Matches Found

### Example 14

The NOV14 clone was analyzed, and the nucleotide and encoded polypeptide sequences are shown in Table 14A.

TABLE 14A
NOV14 Sequence Analysis

| | |
|---|---|
| SEQ ID NO: 45 | 1728 bp |

| NOV14a, | ATGGATCTGGTGCTAAAAAGATGCCTTCTTCATTTGGCTGTGATAGGTGCTTTGCTGG | |
|---|---|---|
| CG158114-01 DNA | | |
| Sequence | CTGTGGGGGCTACAAAAGGGAGCCAGGTGTGGGGAGGACAGCCAGTGTATCCCCAGGA | |
| | AACTGACGATGCCTGCATCTTCCCTGATGGTGGACCTTGCCCATCTGGCTCTTGGTCT | |
| | CAGAAGAGAAGCTTTGTTTATGTCTGOAAGACCTGGGGCCAATACTGGCAGGTTCTAG | |
| | GGGGCCCAGTGTCTGGGCTGAGCATTGGGACAGGCAGGGCAATGCTGGGCACACACAC | |
| | CATGGAAGTGACTGTCTACCATCGCCCGGGATCCCGGAGCTATGTGCCTCTTGCTCAT | |
| | TCCAGCTCAGCCTTCACCATTACTGACCAGGTGCCTTTCTCCGTGAGCGTGTCCCAGT | |
| | TGCGGGCCTTGGATGGAGGGAACAAGCACTTCCTGAGAAATCAGCCTCTGACCTTTGC | |
| | CCTCCAGCTCCATGACCCCAGTGGCTATCTGGCTGAAGCTGACCTCTCCTACACCTGG | |
| | GACTTTGGAGACAGTAGTGGAACCCTGATCTCTCGGGCACTTGTGGTCACTCATACTT | |
| | ACCTGGAGCCTGGCCCAGTCACTGCCCAGGTGGTCCTGCAGGCTGCCATTCCTCTCAC | |
| | CTCCTGTGGCTCCTCCCCAGTTCCAGGCACCACAGATGGGCACAGGCCAACTGCAGAG | |
| | GCCCCTAACACCACAGCTGGCCAAGTGCCTACTACAGAAGTTGTGGGTACTACACCTG | |
| | GTCAGGCGCCAACTGCAGAGCCCTCTCGAACCACATCTGTGCAGGTGCCAACCACTGA | |
| | AGTCATAAGCACTGCACCTGTGCAGATGCCAACTGCAGAGAGCACAGGTATGACACCT | |
| | GAGAAGGTGCCAGTTTCAGAGGTCATGGGTACCACACTGGCAGAGATGTCAACTCCAG | |
| | AGGCTACAGGTATGACACCTGCAGAGGTATCAATTGTGGTGCTTTCTGGAGCCACAGC | |
| | TGCACAGGTAACAACTACAGAGTCGGTGGAGACCACAGCTAGAGAGCTACCTATCCCT | |
| | GAGCCTGAAGGTCCAGATGCCAGCTCAATCATGTCTACGGAAAGTATTACAGGTTCCC | |
| | TGGGCCCCCTGCTGGATGGTACAGCCACCTTAAGGCTGGTGAACAGACAAGTCCCCCT | |
| | GGATTGTGTTCTGTATCGATATGGTTCCTTTTCCGTCACCCTGGACATTGTCCAGGGT | |
| | ATTGAAAGTGCCGAGATCCTGCAGGCTGTGCCGTCCGGTGAGGGGGATGCATTTGAGC | |
| | TGACTGTGTCCTGCCAAGGCGGGCTGCCCAAGGAAGCCTGCATGGAGATCTCATCGCC | |
| | AGGGTGCCAGCCCCCTGCCCAGCGGCTGTGCCAGCCTGTGCTACCCAGCCCAGCCTGC | |
| | CAGCTGGTTCTGCACCAGATACTGAAGGGTGGCTCGGGGACATACTGCCTCGTCGTGT | |
| | CTCTGGCTGATACCAACAGCCTGGCAGTGGTCAGCACCCAGCTTATCATGCCTGGTCA | |
| | ACAAGCAGGCCTTGGGCAGGTTCCGCTGATCGTGGGCATCTCGCTGGTGTTGATGGCT | |
| | GTGGTCCTTGCATCTCTGATATATAGGCGCAGACTTATCAAGCTAGACTTCTCCGTAC | |
| | CCCAGTTGCCACATAGCAGCAGTCACTGGCTGCGTCTACCCCCCATCTTCTGCTCTTG | |
| | TCCCATTGGTGAGAATAGCCCCCTCCTCAGTGGGCAGCAGGTCTGA | |
| | ORF Start: ATG at 1 | ORF Stop: TGA at 1726 |

| | | |
|---|---|---|
| SEQ ID NO: 46 | 575 aa | MW at 60580.6kD |

| | |
|---|---|
| NOV14a, | MDLVLKRCLLHLAVIGALLAVGATKGSQVWGGQPVYPQETDDACIFPDGGPCPSGSWS |
| CG158114 -01 | |
| Protein Sequence | QKRSFVYVWKTWGQYWQVLGGPVSGLSIGTGRAVTGTHTMEVTVYHRRGSRSYVPLAH |
| | SSSAFTITDQVPFSVSVSQLRALDGGNKHFLRNQPLTFALQLHDPSGYLAEADLSYTW |
| | DFGDSSGTLISRALVVTHTYLEPGPVTAQVVLQAAIPLTSCGSSPVPGTTDGHRPTAE |
| | APNTTAGQVPTTEVVGTTPGQAPTAEPSGTTSVQVPTTEVISTAPVQMPTAESTGMTP |
| | EKVPVSEVMGTTLAEMSTPEATGMTPAEVSIVVLSGTTHQVTTTEWVETTARELPIGP |
| | EPEGPDASSIMSTESITGSLGPLLDGTATLRLVKRQVPLDCVLYRYGSFSVTLDIVQG |
| | IESAEILQAVPSGEGDAFELTVSCQGGLPKEACMEISSPGCQPPAQRLCQPVLPSPAC |
| | QLVLHIQILKGGSGTYCLNVSLADTNSLAVVSTQLIMPGQEAGLGQVPLIVGLLVLMA |
| | VVLASLIYRRRLMKQDFSVPQLPHSSSHWLRLPRIFCSCPIGENSPLLSGQQV |

Further analysis of the NOV14a protein yielded the following properties shown in Table 14B.

TABLE 14B
Protein Sequence Properties NOV14a

| | |
|---|---|
| PSort | 0.4600 probability located in plasma membrane; |
| analysis: | 0.1000 probability located in endoplasmic |
| | reticulum (membrane); 0.1000 probability located |
| | in endoplasmic reticulum (lumen); |
| | 0.1000 probability located in outside |
| SignalP | Cleavage site between residues 27 and 28 |
| analysis: | |

A search of the NOV14a protein against the Geneseq database, a proprietary database that contains sequences published in patents and patent publication, yielded several homologous proteins shown in Table 14C.

TABLE 14C
Geneseq Results for NOV14a

| | | NOV14a | Identities/ | |
|---|---|---|---|---|
| | | Residues/ | Similarities | |
| Geneseq | Protein/Organism/Length | Match | for the Matched | Expect |
| Identifier | [Patent #, Date] | Residues | Region | Value |
| AAU09695 | Human melanoma antigen | 26 . . . 575 | 550/550 (100%) | 0.0 |
| | gp100 - Homo sapiens, 661 | 112 . . . 661 | 550/550 (100%) | |
| | aa. [WO200192294-A2, 06 | | | |
| | DEC. 2001] | | | |
| AAU84803 | Human gp100 consensus | 26 . . . 575 | 550/550 (100%) | 0.0 |
| | sequence - Homo sapiens, | 112 . . . 661 | 550/550 (100%) | |
| | 29 NOV. 2001] | | | |
| AAU29003 | Melanoma antigen cDNA25 - | 26 . . . 575 | 550/550 (100%) | 0.0 |
| | Synthetic, 661 aa. | 112 . . . 661 | 550/550 (100%) | |
| | [US6270778-B1, 07 | | | |
| | AUG. 2001] | | | |
| AAB47540 | Human melanoma antigen | 26 . . . 575 | 550/550 (100%) | 0.0 |
| | gp100 - Homo sapiens, 661 | 112 . . . 661 | 550/550 (100%) | |
| | aa. [WO200170767-A2, 27 | | | |
| | SEP. 2001] | | | |
| AAY31977 | Human melanoma antigen | 26 . . . 575 | 550/550 (100%) | 0.0 |
| | gp100 - Homo sapiens, 661 | 112 . . . 661 | 550/550 (100%) | |
| | aa. [WO9947102-A2, 23 | | | |
| | SEP. 1999] | | | |

In a BLAST search of public sequence datbases, the NOV14a protein was found to have homology to the proteins shown in the BLASTP data in Table 14D.

TABLE 14D
Public BLASTP Results for NOV14a

| | | NOV14a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P40967 | Melanocyte protein Pmel 17 | 26 . . . 575 | 550/550 (100%) | 0.0 |
| | precursor (Melanocyte lineage- | 112 . . . 661 | 550/550 (100%) | |
| | specific antigen GP100) | | | |
| | (Melanoma-associated ME20 | | | |
| | antigen) (ME20M/ME20S) (ME20- | | | |
| | M/ME20-S) (95 kDa melanocyte- | | | |
| | specific secreted | | | |
| | glycoprotein) - Homo sapiens | | | |
| | (Human), 661 aa. | | | |
| CAC38954 | Sequence 109 from Patent | 26 . . . 575 | 548/550 (99%) | 0.0 |
| | WO0130382 - synthetic | 112 . . . 661 | 548/550 (99%) | |
| | construct, 661 aa. | | | |
| I38400 | melanoma-associated ME20 | 26 . . . 575 | 550/551 (99%) | 0.0 |
| | antigen (me20m) - human, 662 | 112 . . . 662 | 550/551 (99%) | |
| | aa. | | | |
| A41234 | melanocyte-specific protein | 26 . . . 575 | 549/557 (98%) | 0.0 |
| | Pmel-17 precursor - human, 668 | 112 . . . 668 | 549/557 (98%) | |
| | aa. | | | |
| Q9CZB2 | N/A - Mus musculus (Mouse), | 26 . . . 575 | 415/550 (75%) | 0.0 |
| | 626 aa. | 111 . . . 626 | 448/550 (81%) | |

PFam analysis predicts that the NOV14a protein contains the domains shown in the Table 14E.

TABLE 14E
Domain Analysis of NOV14a

| | | Identities/ | |
|---|---|---|---|
| | | Similarities for | |
| Pfam | NOV14a Match | the Matched | Expect |
| Domain | Region | Region | Value |
| PKD | 131 . . . 215 | 26/99 (26%) | 5.6e−08 |
| | | 61/99 (62%) | |

### Example 15

The NOV15 clone was analyzed, and the nucleotide and encoded polypeptide sequences are shown in Table 15A.

TABLE 15A
NOV15 Sequence Analysis

| | |
|---|---|
| SEQ ID NO: 47 | 1733 bp |

| NOV15a, | CTCGAGCTGCAGAGCTAGCTCTGCAGCTCGCTGCAGAGCTCAGCTGCGTCCGGCGGAG | |
|---|---|---|
| CG158553-01 DNA | | |
| Sequence | GCAGCTGCTGACCCAGCTGTGGACTGTGCCGGGGGCGGGGGACGGAGGGGCAGGAGCC | |
| | CTGGGCTCCCCGTGGCGGGGGCTGTATCATGGACCACCTCGGGGCGTCCCTCTGGCCC | |
| | CAGGTCGCCTCCCTTTGTCTCCTGCTCGCTGGGGCCGCCTGGGCGCCCCCGCCTAACC | |
| | TCCCGGACCCCAAGTTCGAGAGCAAAGCGGCCTTGCTGGCGGCCCGGGGGCCCGAAGA | |
| | GCTTCTGTGCTTCACCGAGCGGTTGGAGGACTTGGTGTGTTTCTGGGAGGAAGCGGCG | |
| | AGCGCTGGGGTGGGCCCGGGCAACTACAGCTTCTCCTACCAGCTCGAGGATGAGCCAT | |
| | GGAAGCTGTGTCGCCTGCACCAGGCTCCCACGGCTCGTGGTGCGGTGCGCTTCTGGTG | |
| | TTCGCTGCCTACACCCGACACGTCGAGCTTCGTGCCCCTAGAGTTGCGCGTCACAGCA | |
| | GCCTCCGGCGCTCCGCGATATCACCGTGTCATCCACATCAATGAAGTAGTGCTCCTAG | |
| | ACGCCCCCGTGGGGCTGGTGGCGCGGTTCGCTGACGAGAGCGGCCACGTAGTGTTGCG | |
| | CTGGCTCCCGCCGCCTGACACACCCATGACGTCTCACATCCGCTACGAGGTGGACGTC | |
| | TCGGCCGGCAACGGCGCAGGGAGCGTACAGAGGGTCGAGATCCTGGAGGGCCGCACCG | |
| | AGTGTGTGCTGAGCAACCTGCGGGGCCGGACGCGCTACACCTTCGCCGTCCGCGCGCG | |
| | TATGGCTGAGCCGAGCTTCGGCGGCTTCTGGAGCGCCTGGTCGGAGCCTGTGTCGCTG | |
| | CTGACGCCTAGCGACCTGGACCCCCTCATCCTGACGCTCTCCCTCATCCTCGTGGTCA | |
| | TCCTGGTGCTGCTGACCGTGCTCGCGCTGCTCTCCCACCGCCGGGCTCTGAAGCAGAA | |
| | GATCTGGCCTGGCATCCCGAGCCCAGAGAGCGAGTTTGAAGGCCTCTTCACCACCCAC | |
| | AAGGGTAACTTCCAGCTGTGGCTGTACCAGAATGATGGCTGCCTGTGGTGGAGCGCCT | |
| | GCACCCCCTTCACCGAGGACCCACCTGCTTTCCTGGAAGTCCTCTCAGAGCGCTGCTG | |
| | GGGGACGATGCAGGCAGTGGAGCCGGGOACAGATGATGAGCGCCCCCTGCTGGAGCCA | |
| | GTGGGCAGTGAGCATGCCCAGGATACCTATCTGGTGCTGGACAAATGGTTGCTGCCCC | |
| | GGAACCCGCCCAGTGAGGACCTCCCAGGGCCATGGGCACTGTGCCCTGAGcTGCcCCC | |
| | TACCCCACCCCACCTAAAGTACCTGTACCTTGTGGTATCTGACTCTGGCATCTCAACT | |
| | GACTACAGCTCAGGGGACTCCCAGGGAGCCCAAGGGGGCTTATCCGATGCCCCCTACT | |
| | CCAGCCCTTATGAGAACAGCCCTATCCCAGCCGCTGAGCCTCTGCCCCCCAGCTATGT | |
| | GGCTTGCTCTTAGGACACCAGGCTGCAGATGATCAGGGATCCAATATGACTCAGAGAA | |
| | CCAGTGCAGACTCAAGACTTATGGAACAGGGATGGCGAGGCCTCTCTCAGGAGCAGGG | |
| | GCATTGCTGATTTTGTCTGCCCAATCCATCCTGCTCAGGAAACCACAACCTTGCAGTA | |
| | TTTTTAAATATGTATAGTTTTTTTGCTGCAGAGCTAGCTCTGCAGCTCGAG | |
| | ORF Start: ATG at 145 | ORF Stop: TAG at 1519 |

| | | |
|---|---|---|
| SEQ ID NO: 48 | 458 aa | MW at 50069.3kD |

| NOV15 a, | MDHLGASLWPQVGSLCLLLAGAAWAPPPNLPDPKFESKAALLASGPEELLCFTRERLE | |
|---|---|---|
| CG158553-01 | | |
| Protein Sequence | DLVCFWEEAASAGVGPGNYSFSYQLEDEPWKLCRLHQAPTFEGAVRFWCSLPTADTSS | |
| | FVPLELRVTSGSGAPRYHRVIHINEVVLLDAPVGLVARLADESGHRALRWLPPPETPM | |
| | TSHIRYEVDVSAGNGAGSVQRVEILEGRTECVLSNLRGRTRYTFAVRARMAEPSFGGF | |
| | WSAWSEPVSLLTPSDLDPLILTLSLILVVILVLLTVLALLSHRPSAKQKIWPGIPSPE | |
| | SEFEGLFTTHKGNFQLWLYQNDGCLWWSACTPFTEDPPAFLEVLSERCWGTMQAVEPG | |
| | TDDEGPLLEPVGSEHAQDTYLVLDKWLLPRNPPSEDLPGPWALCPELPPTPPHLKYLY | |
| | LVVSDSGISTDYSSGDSQGAQGGLSDGPYSSPYENSPIPAAEPLPPSYVACS | |
| | SEQ ID NO: 49 | 1733 bp |

| NOV15b, | CTCGAGCTGCAGAGCTAGCTCTGCAGCTCGCTGCAGAGCTCAGCTGCGTCCGGCGGAG | |
|---|---|---|
| CG158553-01 DNA | | |
| Sequence | GCAGCTGCTGACCCAGCTGTGGACTGTGCCGGGGGCGGGGGACGGAGGGGCAGGAGCC | |
| | CTGGGCTCCCCGTGGCGGGGGCTGTATCATGGACCACCTCGGGGCGTCCCTCTGGCCC | |
| | CAGGTCGGCTCCCTTTGTCTCCTGCTCGCTGGGGCCGCCTGGGCGCCCCCGCCTGACC | |
| | TCCCGGACCCCAAGTTCGAGAGCAAAGCGGCCTTGCTGGCGGCCCGGGGGCCCGAAGA | |
| | GCTTCTGTGCTTCACCGAGCGGTTGGAGGACTTGGTGTGTTTCTGGGAGGAGGCGGCG | |
| | AGCGCTGGGGTGGGCCCGGGCAACTACAGCTTCTCCTACCAGCTCGAGGATGAGCCAT | |
| | GGAAGCTGTGTCGCCTCCACCAGGCTCCCACGGCTCGTGGTGCGGTGCGCTTCTGGTG | |
| | TTCGCTGCCTACAGCCGACACGTCGAGCTTCGTGCCCCTAGAGTTGCGCGTCACAGCA | |
| | GCCTCCGGCGCTCCGCGATATCACCGTGTCATCCACATCAATGAAGTAGTGCTCCTAG | |
| | ACGCCCCCGTGGGGCTGGTGGCGCGGTTGGCTGACGAGAGCGGCCACGTAGTGTTGCG | |
| | CTGGCTCCCGCCGCCTGAGACACCCATGACGTCTCACATCCGCTACGAGGTGGACGTC | |
| | TCGGCCGGCAACGGCGCAGGGAGCGTACAGAGGGTGGAGATCCTGGAGGGCCGCACCG | |
| | AGTGTGTGCTGAGCAACCTGCGGGGCCGGACGCCCTACACCTTCGCCGTCCGCGCGCG | |
| | TATGGCTGAGCCGAGCTTCGGCGGCTTCTGGAGCGCCTGGTCGGAGCCTGTGTCGCTG | |
| | CTGACGCCTAGCGACCTGGACCCCCTCATCCTGACGCTCTCCCTCATCCTCGTGGTCA | |
| | TCCTGGTGCTGCTGACCGTGCTCGCGCTGCTCTCCCACCGCCGGGCTCTGACGCAGAC | |
| | GATCTGGCCTGGCATCCCGAGCCCAGAGAGCGAGTTTGAAGGCCTCTTCACCACCCAC | |
| | AAGGGTAACTTCCAGCTGTGGCTGTACCAGAATGATGGCTGCCTGTGGTGGAGCGCCT | |
| | GCACCCCCTTCACGGAGGACCCACCTGCTTTCCTGGAAGTCCTCTCAGAGCGCTGCTG | |
| | GGGGACGATGCAGGCAGTGGAGCCGGGGACAGATGATGAGGGCCCCCTGCTGGAGCCA | |
| | GTGGGCAGTGAGCATGCCCAGGATACCTATCTGGTGCTGGACAAATGGTTGCTGCCCC | |
| | GGAACCCGCCCAGTGAGGACCTCCCAGGGCCATGGGCACTGTGCCCTGAGCTGCCCCC | |
| | TACCCCACCCCACCTAAAGTACCTGTACCTTGTGGTATCTGACTCTGGCATCTCGACT | |
| | GACTACAGCTCAGGGGACTCCCAGGGAGCCCAAGGGGGCTTATCCGATGGCCCCTACT | |
| | CCAGCCCTTATGAGAACAGCCCTATCCCAGCCGCTGAGCCTCTGCCCCCCAGCTATGT | |
| | GGCTTGCTCTTAGGACACCAGGCTGCAGATGATCAGGGATCCAATATGACTCAGAGAT | |
| | CCAGTGCAGACTCAAGACTTATGGAACAGGGATGGCGAGGCCTCTCTCAGGAGCAGGG | |
| | GCATTGCTGATTTTGTCTGCCCAATCCATCCTGCTCAGGAAACCACAACCTTGCAGTA | |
| | TTTTTAAATATGTATAGTTTTTTTGCTGCAGAGCTAGCTCTGCAGCTCGAG | |
| | ORF Start: ATG at 145 | ORF Stop: TAG at 1519 |

| | | |
|---|---|---|
| SEQ ID NO: 50 | 458 aa | MW at 50069.3kD |

| NOV15b, | MDHLGASLWPQVGSLCLLLAGAAWAPPPNLPDPKFESKAALLAGTGPEELLCFTERLE | |
|---|---|---|
| CG158553-01 | | |
| Protein Sequence | DLVCFWEEAASAGVGPGNYSFSYQLEDEPWKLCRLHQAPTARGAVRFWCSLPTADTSS | |
| | FVPLELRVTAASGAPRYHRVIHINEVVLLDAPVGLVARLADESGHNRLRWLPPPETPM | |
| | TSHRYEVDVSAGNGAGSVQRVEILEGRTECVLSNLRGRTRYTFAVRARMAEPSRFGGF | |
| | WSAWSEPVSLLTPSDLDPLILTLSLILVVILVLLTVLALLSHRRALKQKIWPGIPSPE | |
| | SEFEGLFTTHKGNFQLWLYQNDGCLWWSACTPFTEDPPAFLEVLSERCWGTMQAVEPG | |
| | TDDEGPLLEPVGSEHAQDTYLVLDKWLLPRNPPSEDLPGPWALCPELPPTPPHLKYLY | |
| | LVVSDSGISTDYSSGDSQGAQGGLSDGPYSSPYENSPIPAAEPLPPSYVACS | |
| | SEQ ID NO: 51 | 1435 bp |

| NOV15c, | GGGGCTGTATCATGGACCACCTCGGGGCGTCCCTCTGGCCCCAGGTCGGCTCCCTTTG | |
|---|---|---|
| CG158553-02 DNA | | |
| Sequence | TCTCCTGCTCGCTGGGGCCGCCTGGGCGCCCCCGCCTAACCTCCCGGACCCCTAGTTC | |
| | GAGAGCAAAGCGGCCTTGCTGGCGGCCCGGGGGCCCGAAGAGCTTCTGTGCTTCACCG | |
| | ACCGGTTGGGGCACTTGGTGTGTTTCTGGGAGGAAGCGGCGAGCGCTGGGGTGGGCCC | |
| | GGGCAACTACAGCTTCTCCTACCAGCTCGAGGATGAGCCATGGCTGCTGTGTCGCCTG | |
| | CACCAGGCTCCCACGGCTCGTGGTCCGGTGCGCTTCTGGTGCTCGCTGCCTACACCCG | |
| | ACACGTCGAGCTTCGTGCCCCTAGAGTTGCGCGTCACAGCAGCCTCCGGCGCTCCGCG | |
| | ATATCACCGTGTCATCCACATCAATGAAGTAGTGCTTCTAGACGCCCCCGTGGGGCTG | |
| | GTGGCGCGGTTGGCTGACGAGAGCGGCCACGTAGTGTTGCGCTGGCTCCCGCCGCCTG | |
| | AGACACCCATGACGTCCCACATCCGCTACGAGGTGGACGTCTCGGCCGGCGTCGGCGC | |
| | AGGGAGCGTACAGAGGGTGGAGATCCTGGAGGGCCGCACCGAGTGTGTGCTGAGCTAC | |
| | CTGCGGGGCCGGACGCGCTACACCTTCGCCGTCCGCACGCGTATGGCTGAGCCGAGCT | |
| | TCGGCGGCTTCTGGAGCGCCTGGTCGGAGCCTGTGTCGCTGCTGACGCCTAGCGACCT | |
| | GGACCCCCTCATCCTGACGCTCTCCCTCATCCTCGTGGTCATCCTGGTGCTGCTGACC | |
| | GTGCTCGCGCTGCTCTCCCACCGCCGGGCTCTGAAGCAGAAGATCTGGCCTGGCATCC | |
| | CGAGCCCAGAGAGCGAGTTTGAAGGCCTCTTCACCACCCACAAGGGTATCTTCCAGCT | |
| | GTGGCTGTACCAGAATGATGGCTGCCTGTGGTGGACCCCCTGCACCCCCTTCACGGAG | |
| | GACCCACCTGCTTCCCTGGAAGTCCTCTCAGAGCGCTGCTGGGGGACGATGCAGGCAG | |
| | TGGAGCCGGGGACAGATGATGAGGGCCCCCTGCTGGAGCCAGTGGGCAGTGAGCATGC | |
| | CCAGGATACCTATCTGGTGCTGGACAAATGGTTGCTGCCCCGGAACCCGCCCAGTGAG | |
| | GACCTCCCAGGGCCATGGGCACTGTGCCCTGAGCTGCCCCCTACCCCACCCCACCTCG | |
| | AGTACCTGTACCTTGTGGTATCTGACTCTGGCATCTCAACTGACTACAGCTCAGGGGA | |
| | CTCCCAGGGAGCCCAAGGGGGCTTATCCGATGGCCCCTACTCCAGCCCTTATGAGTAC | |
| | AGCCCTATCCCAGCCGCTGAGCCTCTGCCCCCCAGCTATGTGGCTTGCTCTTAGGACA | |
| | CCAGGCTOCAGATGATCAGGGATCCAATATGACTCAGAGAACC | |
| | ORF Start: ATG at 12 | ORF Stop: TAG at 1386 |

| | | |
|---|---|---|
| SEQ ID NO: 52 | 458 aa | MW at 49993.2kD |

| NOV15a, | MDHLGASLWPQVGSLCLLLAGAAWAPPPNLPDPKFESKAALLAARGPEELLCFTERLG | |
|---|---|---|
| CG158553-02 | | |
| Protein Sequence | DLVCFWEEAASAGVGPGNYSFSYQLEDEPWKLCRLHQAPTARGAVRFWCSLPTADTSS | |
| | FVPLELRVTAASGAPRYHRVIHINEVVLLDAPVGLVARLADESGHVVLRWLPPPETPM | |
| | TSHIRYEVDVSAGNGAGSVQRVEILEGRTECVLSNLRGRTRYTFAVRTRMAEPSFGGF | |
| | WSAWSEPVSLLTPSDLDPLILTLSLILVVILVLLTVLALLSHRRALKQKTWPGIPSPE | |
| | SEFEGLFTTHKGNFQLWLYQNDGCLWWSPCTPFTEDPPASLEVLSERCWGTMQAVEPG | |
| | TDDEGPLLEPVGSEHAQDTYLVLDKWLLPRNPPSEDLPGPWALCPELPPTPPHLKYLY | |
| | LVVSDSGISTDYSSGDSQGAQGGLSDGPYSSPYENSPIPAAEPLPPSYVACS | |
| | SEQ ID NO: 53 | 1585 bp |

| NOV15d, | GGGGCTGTATCATGGACCACCTCGGGGCGTCCCTCTGGCCCCAGGTCGGCTCCCTTTG | |
|---|---|---|
| CG158553-03 DNA | | |
| Sequence | TCTCCTGCCCGCTGGGGCCGCCTGGGCGCCCCCGCCTAACCTCCCGGACCCCAAGTTC | |
| | GAGAGCAAAGCGGCCTTOCTGGCGGCCCGGGGGCCCGAAGAGCTTCTGTGCTTCACCG | |
| | AGCGGTTGGAGGACTTGGTGTGTTTCTGGGAGGAAGCGGCGAGCGCTGGGGTGGGCCC | |
| | GGGCAACTACAGCTTCTCCTACCAGCTCGAGGATGAGCCATGGAAGCTGTGTCGCCTG | |
| | CACCAGGCTCCCACGGCTCGTGGTGCGGTGCGCTTCTGGTGTTCGCTGCCTACAGCCG | |
| | ACACGTCGAGCTTCGTGCCCCTAGAGTTGCGCGTCACAGCAGCCTCCGGCGCTCCGCG | |
| | ATATCACCGTGTCATCCACATCAATGAAGTAGTGCTCCTAGACGCCCCCGTGGGGCTG | |
| | GTGCCGCGGTTGGCTGACGAGAGCGGCCACGTAGTGTTGCGCTGGCTCCCCCCGCCTG | |
| | AGACACCCATGACGTCTCACATCCGCTACGCGGTGGACGTCTCGGCCGGCGACGGCGC | |
| | AGGGAGCGTACAGAGGGTGAAGATCCTGGAGGGCCGCACCGAGTGTGTGCTGAGCGTC | |
| | CTGCGGGGCCGGACGCGCTACACCTTCGCCGTCCGCGCGCGTATGGCTGAGCCGAGCT | |
| | TCGGCGGCTTCTGGAGCGCCTGGTCGGAGCCTGTGTCGCTCCTGACGCCTAGCGACCT | |
| | GGACCCCCTCATCCTGACGCTCTCCCTCATCCTCGTGGTCATCCTGGTGCTGCTGACC | |
| | GTGCTCGCGCTGCTCTCCCACCGCCGGGCTCTGAAGCAGAAGATCTGGCCTGGCATCC | |
| | CGAGCCCAGAGAGCGAGTTTGAAGGCCTCTTCACCACCCACAAGGGTAACTTCCAGCT | |
| | GTGGCTGTACCAGAATGATGGCTGCCTGTGGTGGAGCCCCTGCACCCCCTTCACGGAG | |
| | GACCCACCTGCTTCCCTGGAAGTCCTCTCAGAGCGCTGCTGGGGGACGATGCAGGCAG | |
| | TGGAGCCGGGGACAGATGATGAGGGCCCCCTGCTGGAGCCAGTGGGCAGTCAGCATGC | |
| | CCAGGATACCTATCTGGTGCTGGACAAATGGTTGCTGCCCCGGAACCCGCCCAGTGAG | |
| | GACCTCCCAGGGCCTGGTGOCAGTGTGGACATAGTGGCCATGGATGAAGGCTCAGTAG | |
| | CATCCTCCTGCTCATCTGCTTTGGCCTCGAAGCCCAGCCCAGAGGGAGCCTCTCCTGC | |
| | CAGCTTTGAGTACACTATCCTGGACCCCAGCCCCCAGCTCTTGCGTCCATGGACACTG | |
| | TGCCCTGAGCTGCCCCCTACCCCACCCCACCTAAAGTACCTGTACCTTGTGGTATCTG | |
| | ACTCTGGCATCTCAACTGACTACAGCTCAGGGGACTCCCAGGGAGCCCTCGGGGGCTT | |
| | ATCCGATGGCCCCTACTCCAACCCTTATGAGAACAGCCTTATCCCAGCCGCTGAGCCT | |
| | CTGCCCCCCAGCTATGTGGCTTGCTCTTAGGACACCAGGCTGCAGATGATCAGGGATC | |
| | CAATATGACTCAGAGAACC | |
| | ORF Start: ATG at 12 | ORF Stop: TAG at 1536 |

| | | |
|---|---|---|
| SEQ ID NO: 54 | 508 aa | MW at 54999.6kD |

| | |
|---|---|
| NOV15d, | MDHLGASLWPQVGSLCLLPAGAAWAPPPNLPDPKFESKAALLAARGPEELLCFTERLE |
| CG158553-03 | |
| Protein Sequence | DLVCFWEEAASAGVGPGNYSFSYQLEDEPWKLCRLHQAPTARGAGREFWCSLPTADTS |
| | FVPLELRVTAHASGAPRYHRVIHINEVVLLDAPVGLVKARLADESGHLRWLPPPETPM |
| | TSHTRYAVDVSAGNGAGSVQRVKILEGRTECVLSNLRGRTRYTFAVRARMAEPSFGGF |
| | WSAWSEPVSLLTPSDLDPLILTLSLILVVILVLLTVLALLSHRPALKQKIWPGIPSPE |
| | SEFEGLFTTHKGNFQLWLYQNDGCLWWSPCTPFTEDPPASLEVLSERCWGTMQAVEPG |
| | TDDEOPLLEPVGSEHAQDTYLVLDKWLLPRNPPSEDLPGPGGSVDIVIDEGSKEASSC |
| | SSALASKPSPEGASAASFEYTILDPSPQLLRPWTLCPELPPTPKPHLKYLYLTSDSGI |
| | STDYSSGDSQGAQGGLSDGPYSNPYENSLTPAAEPLPPSYVACS |

Sequence comparison of the above protein sequences yields the following sequence relationships shown in Table 15B.

TABLE 15B
Comparison of NOV15a against NOV15b through NOV15d.

| | | Identities/ |
|---|---|---|
| | | Similarities for |
| Protein | NOV15a Residues/ | the Matched |
| Sequence | Match Residues | Region |
| NOV15b | 1 . . . 458 | 458/458 (100%) |
| | 1 . . . 458 | 458/458 (100%) |
| NOV15c | 1 . . . 458 | 454/458 (99%) |
| | 1 . . . 458 | 454/458 (99%) |
| NOV15d | 1 . . . 458 | 450/508 (88%) |
| | 1 . . . 508 | 452/508 (88%) |

Further analysis of the NOV15a protein yielded the following properties shown in Table 15C.

TABLE 15C
Protein Sequence Properties NOV15a

| | |
|---|---|
| PSort | 0.4600 probability located in plasma membrane; |
| analysis: | 0.1762 probability located in microbody |
| | (peroxisome); 0.1000 probability located in |
| | endoplasmic reticulum (membrane); 0.1000 |
| | probability located in endoplasmic reticulum (lumen) |
| SignalP | Cleavage site between residues 26 and 27 |
| analysis: | |

A search of the NOV15a protein against the Geneseq database, a proprietary database that contains sequences published in patents and patent publication, yielded several homologous proteins shown in Table 15D.

TABLE 15D
Geneseq Results for NOV15a

| | | NOV15a | Identities/ | |
|---|---|---|---|---|
| | | Residues/ | Similarities | |
| Geneseq | Protein/Organism/Length | Match | for the | Expect |
| Identifier | [Patent #, Date] | Residues | Hatched Region | Value |
| AAR69503 | Human erythropoietin | 1 . . . 458 | 453/508 (89%) | 0.0 |
| | receptor - Homo sapiens, 508 | 1 . . . 508 | 454/508 (89%) | |
| | aa. [US5378808-A, 03 JAN. | | | |
| | 1995] | | | |
| AAR70032 | Human erythropoietin | 1 . . . 458 | 453/508 (89%) | 0.0 |
| | receptor - Homo sapiens, 508 | 1 . . . 508 | 454/508 (89%) | |
| | aa. [WO9505469-A, 23 FEB. | | | |
| | 1995] | | | |
| AAR06512 | EPO receptor - Homo sapiens, | 1 . . . 458 | 453/508 (89%) | 0.0 |
| | 508 aa. [WO9008822-A, 09 | 1 . . . 508 | 454/508 (89%) | |
| | AUG. 1990] | | | |
| ABB09173 | Human erythropoietin | 1 . . . 458 | 452/508 (88%) | 0.0 |
| | receptor SEQ ID NO:5 - Homo | 1 . . . 508 | 453/508 (88%) | |
| | sapiens, 508 aa. | | | |
| | [US2002031806-A1, 14 MAR. | | | |
| | 2002] | | | |
| AAY44622 | Truncated human EpoR (t439) - | 1 . . . 388 | 386/388 (99%) | 0.0 |
| | Homo sapiens, 438 aa. | 1 . . . 388 | 386/388 (99%) | |
| | [W09967360-A2, 29 DEC. | | | |
| | 1999] | | | |

In a BLAST search of public sequence datbases, the NOV 15a protein was found to have homology to the proteins shown in the BLASTP data in Table 15E.

TABLE 15E
Public BLASTP Results for NOV15a

| | | NOV15a | Identities/ | |
|---|---|---|---|---|
| Protein | | Residues/ | Similarities | |
| Accession | | Match | for the Matched | Expect |
| Number | Protein/Organism/Length | Residues | Portion | Value |
| P19235 | Erythropoietin receptor | 1 . . . 458 | 453/508 (89%) | 0.0 |
| | precursor (EPO-R) - Homo | 1 . . . 508 | 454/508 (89%) | |
| | sapiens (Human), 508 aa. | | | |
| Q9MYZ9 | Erythropoietin receptor - | 1 . . . 458 | 386/509 (75%) | 0.0 |
| | Sus scrofa (Pig), 509 aa. | 1 . . . 509 | 402/509 (78%) | |
| P14753 | Erythropoietin receptor | 1 . . . 458 | 375/508 (73%) | 0.0 |
| | precursor (EPO-R) - Mus | 1 . . . 507 | 397/508 (77%) | |
| | musculus (Mouse), 507 aa. | | | |
| AAH03953 | Similar to erythropoietin | 2 . . . 458 | 374/507 (73%) | 0.0 |
| | receptor - Mus musculus | 1 . . . 506 | 396/507 (77%) | |
| | (Mouse), 506 aa (fragment). | | | |
| Q07303 | Erythropoietin receptor | 1 . . . 458 | 371/508 (73%) | 0.0 |
| | precursor (EPO-R) - Rattus | 1 . . . 507 | 399/508 (78%) | |
| | norvegicus (Rat), 507 aa. | | | |

PFam analysis predicts that the NOV 15a protein contains the domains shown in the Table 15F.

TABLE 15F
Domain Analysis of NOV15a

| | | Identities/ | |
|---|---|---|---|
| | | Similarities for | |
| Pfam | NOV15a Match | the Matched | Expect |
| Domain | Region | Region | Value |
| fn3 | 145 . . . 228 | 21/88 (24%) | 0.00059 |
| | | 59/88 (67%) | |

### Example 16

The NOV16 clone was analyzed, and the nucleotide and encoded polypeptide sequences are shown in Table 16A.

TABLE 16A
NOV16 Sequence Analysis

| | |
|---|---|
| SEQ ID NO: 55 | 751 bp |

| NOV16 a, | CGCGGCAGCTCCCACCATGGCGGAGACCAAGCTCCAGCTGTTTGTCAAGGCGAGTGAG | |
|---|---|---|
| CG158983-01 DNA | | |
| Sequence | GACGGGGAGAGCGTGGGTCACTGCCCCTCCTGCCAGCGGCTCTTCATGGTCCTGCTCC | |
| | TCAAGGGCGTACCTTTCACCCTCACCACGGTGGACACGCGCAGGTCCCCGGACGTGCT | |
| | GAAGGACTTCGCCCCCGGCTCGCAGCTGCCCATCCTCCTCTATGACAGCGACGCCAAG | |
| | ACAGACACGCTGCAGATCGAGGACTTTCTGGAGGAGACGCTGGGGCCGCCCGAGGAGT | |
| | CCAACACCGCCGGCAACGACGTTTTCCACAAGTTCTCCGCGTTCATCAAGAACCCGGT | |
| | GCCCGCGCAGGACGAAGCCCTGTACCAGCAGCTGCTGCGCGCCCTCGCCAGGCTGGAC | |
| | AGCTACCTGCGCGCGCCCCTGGAGCACGAGCTGGCGGGGGAGCCGCAGCTGCGCGAGT | |
| | CCCGCCGCCGCTTCCTGGACGGCGACAGGCTCACGCTGGCCGACTGCAGCCTCCTGCC | |
| | CAAGCTGCACATCGTCGACACGGTGTGCGCGCACTTCCGCCAGGCGCCCATCCCCGCG | |
| | GAGTGCGCGGCGTACGCCGTTACCTGGACAGCGCGATGCAGGAGAAAGAGTTCAAATA | |
| | CACGTGTCCGCACAGCGCCGAGATCCTGGCGGCCTACCGGCCCGCCGTGCACCCCACG | |
| | CTAGCGCCCCACCCCGCGTCTGTCGCCCAATAAAGGCATCTTTGTCGGOAAAAAA | |
| | ORF Start: ATG at 17 | ORF Stop: TAG at 698 |

| | | |
|---|---|---|
| SEQ ID NO: 56 | 227 aa | MW at 25431.7kD |

| NOV16 a, | MAETKLQLFVKASEDGESVGHCPSCQRLFMVLLLKGVPFTLTTVDTRRSPDVLKDFAP | |
|---|---|---|
| CG158983-01 | | |
| Protein Sequence | GSQLPILLYDSDAKTDTLQIEDPLEETLGPPEESNTAGNDVFHKFSAFIKNPVPAQDE | |
| | ALYQQLLRALARLDSYLRAPLEHELAGEPQLRESRRRFLDGDRLTLADCSLLPKLHIV | |
| | DTVCAHFRQAPIPAECAAYAVTWTARCRRKSSNTRVRTAPRSWRPTGPPCTPR | |
| | SEQ ID NO: 57 | 693 bp |

| NOV16b, | CCCACCATGGCGGAGACCAAGCTCCAGCTGTTTGTCAAGGCGAGTGAGGACGGGGAGA | |
|---|---|---|
| CG158983-02 DNA | | |
| Sequence | GCGTGGGTCACTGCCCCTCCTGCCAGCGGCTCTTCATGGTCCTGCTCCTCAAGGGCGT | |
| | ACCTTTCACCCTCACCACGGTCGACACGCGCAGGTCCCCGGACGTGCTGKTGGACTTC | |
| | GCCCCCGGCTCGCAGCTGCCCATCCTGCTCTATGACAGCGACGCCAGACAGAGCACGC | |
| | TGCAGATCGAGGACTTTCTGGAGGAGACGCTGGGGCCGCCCGAGGAGTCCGACACCGC | |
| | CGGCAACGACGTTTTCCACAAGTTCTCCGCGTTCATCAAGAGCCCGGTGCCCGCGCAG | |
| | GACGAAGCCCTGTACCAGCAGCTGCTGCCCGCCCTCGCCAGGCTGGACAGCTACCTGC | |
| | GCGCGCCCCTGGAGCACGAGCTGGCGGGGGAGCCGCAGCTGCGCGAGTCCCGCCGCCG | |
| | CTTCCTGGACGGCGACAGGCTCACGCTGGCCGACTGCAGCCTCCTGCCCAGGCTGCAC | |
| | ATCGTCGACACGGTGTGCGCGCACTTCCGCCAGGCGCCCATCCCCGCGGAGCTGCGCG | |
| | GCGTACGCCGCTACCTGGACACCGCGATGCAGGAGAAAGAGTTCACGTACACGTGTCC | |
| | GCACAGCGCCGAGATCCTGGCGGCCTACCGGCCCGCCGTGCACCCCCGCTAGCGC | |
| | ORF Start: ATG at 7 | ORF Stop: TAG at 688 |

| | | |
|---|---|---|
| SEQ ID NO: 58 | 227 aa | MW at 25573.8kD |

| NOV16b, | AETKLQLFVKASEDGESVGHCPSCQRLFMVLLLKGVPFTLTTVDTRRSPDGKTKDFAP | |
|---|---|---|
| CG158983-02 | | |
| Protein Sequence | GSQLPILLYDSDAKTDTLQIEDFLEETLGPPEESNTAGNDVFHKFSAFITKPVPAQDE | |
| | ALYQQLLRALARLDSYLRAPLEHELAGEPQLRESRRRFLDGDRLTLADCSLLPKLHIV | |
| | DTVCAHFRQAPIPAELRGVRRYLDSAMQEKEFKYTCPHSAEILAAYRPAVHPR | |
| | SEQ ID NO: 59 | 784 bp |

| NOV16c, | CGGCCGCGTCGACGCGGCAGCTCCCACCATGGCGGAGACCGTGCTCCAGCTGTTTGTC | |
|---|---|---|
| CG158983-03 DNA | | |
| Sequence | AAGGCGAGTGAGGACGGGGAGAGCGTGCGTCACTGCCCCTCCTGCCAGCGGCTCTTCA | |
| | TGGTCCTGCTCCTCAAGGGCGTACCTTTCACCCTCACCACGGTGGACACGCGCAGGTC | |
| | CCCGGACGTGCTGAAGGACTTCGCCCCCGGCTCGCAGCTGCCCATCCTGCTCTATGAC | |
| | AGCGACGCCAAGACAGACACGCTGCAGATCGAGGACTTTCTGGAGGAGACGCTGGGGC | |
| | CGCCCGAGGAGTCCAACACCGCCGGCAACGACGTTTTCCACAAGTTCTCCGCGTTCAT | |
| | CAAGAACCCGGTGCCCGCGCAGGACGAAGCCCTGTACCAGCAGCTGCTGCGCGCCCTC | |
| | GCCAGGCTGGACAGCTACCTGCGCGCGCCCCTGGAGCACGAGCTGGCGGGGGAGCCGC | |
| | AGCTGCGCGAGTCCCGCCGCCGCTTCCTGGACGGCGACAGGCTCACGCTGGCCGACTG | |
| | CAGCCTCCTGCCCAAGCTGCACATCGTCGACACGGTGTGCGCGCACTTCCGCCAGGCG | |
| | CCCATCCCCGCGGAGCTGCGCGGCGTACGCCGCTACCTGGACAGCGCGATGCAGGAGA | |
| | AAGAGTTCAAATACACGTGTCCGCACAGCGCCGAGATCCTGGCGGCCTACCGGCCCGC | |
| | CGTGCACCCCCGCTAGCGCCCCACCCCGCGTCTGTCGCCCAATAAAGGCATCTTTGTC | |
| | GGGAAAAAAAAAAAAAAAAAATTAAAAAAA | |
| | ORF Start: ATG at 29 | ORF Stop: TAG at 710 |

| | | |
|---|---|---|
| SEQ ID NO: 60 | 227 aa | MW at 25573.8kD |

| NOV16c, | MAETKLQLFVKASEDGESVGHCPSCQRLFMVLLLKGVPFTLTTVDTRRSPDVLKDFAP | |
|---|---|---|
| CG158983-03 | | |
| Protein Sequence | GSQLPILLYDSDAKTDTLQIEDFLEETLGPPEESNTAGNDVFHKFSAFIKNPVPAQDE | |
| | ALYQQLLRALARLDSYLRAPLEHELAGEPQLRESRRRFLDGDRLTLADCSLLPKLHIV | |
| | DTVCAHFRQAPIPAELRGVRRYLDSAMQEKEFKYTCPHSAEILAAYRPAVHPR | |
| | SEQ ID NO: 61 | 751 bp |

| NOV16d, | CGCGGCAGCTCCCACCATGGCGGAGACCAAGCTCCAGCTGTTTGTCGAGGCGAGTGAG | |
|---|---|---|
| CG158983-01 DNA | | |
| Sequence | GACGGGGAGAGCGTGGGTCACTGCCCCTCCTGCCAGCGGCTCTTCATGGTCCTGCTCC | |
| | TCAAGGGCGTACCTTTCACCCTCACCACGGTGGACACGCGCAGGTCCCCGGACGTGCT | |
| | GAAGGACTTCGCCCCCGGCTCGCAGCTGCCCATCCTGCTCTATGACAGCGACGCCTCG | |
| | ACAGACACGCTGCAGATCGAGGACTTTCTGGAGGAGACGCTGGGGCCGCCCGAGGAGT | |
| | CCAACACCGCCGGCAACGACGTTTTCCACAAGTTCTCCGCGTTCATCAAGAACCCGGT | |
| | GCCCGCGCAGGACGAAGCCCTGTACCAGCAGCTGCTGCGCGCCCTCGCCAGGCTGGAC | |
| | AGCTACCTGCGCGCGCCCCTGGAGCACGAGCTGGCGGGGGAGCCGCAGCTGCGCGAGT | |
| | CCCGCCGCCGCTTCCTGGACGGCGACAGGCTCACGCTGGCCGACTGCAGCCTCCTGCC | |
| | CAAGCTGCACATCGTCGACACGGTGTGCGCGCACTTCCGCCAGGCGCCCATCCCCGCG | |
| | GAGTGCGCGGCGTACGCCGTTACCTGGACAGCGCGATGCAGGAGAAAGAGTTCAAATA | |
| | CACGTGTCCGCACAGCGCCGAGATCCTGGCGGCCTACCGGCCCGCCGTGCACCCCACG | |
| | CTAGCGCCCCACCCCGCGTCTGTCGCCCAATAAAGGCATCTTTGTCGGGAAAAAA | |
| | ORF Start: ATG at 17 | ORF Stop: TAG at 698 |

| | | |
|---|---|---|
| SEQ ID NO: 62 | 227 aa | MW at 25431.7kD |

| NOV16d, | MAETKLQLFVKASEDGESVGHCPSCQRLFMVLLLKGVPFTLTTVDTRRSPDVLKDFAP | |
|---|---|---|
| CG158983-01 | | |
| Protein Sequence | GSQLPILLYDSDAKTDTLQIEDFLEETLGPPEESNTAGNDVFHKFSAFIKNPVPAQDE | |
| | ALYQQLLRALARLDSYLRAPLEHELAGEPQLRESRRRFLDGDRLTLADCSLLPKLHIV | |
| | DTVCAHFRQAPIPAECAAYAVTWTARCRRKSSNTRVRTAPRSWRPTGPPCTPR | |
| | SEQ ID NO: 63 | 751 bP |

| NOV16e, | CGCGGCAGCTCCCACCATGGCGGAGACCAAGCTCCAGCTGTTTGTCGAGGCGAGTGAG | |
|---|---|---|
| CG158983-01 DNA | | |
| Sequence | GACGGGGAGAGCGTGGGTCACTGCCCCTCCTGCCAGCGGCTCTTCATGGTCCTGCTCC | |
| | TCAAGGGCGTACCTTTCACCCTCACCACGGTGGACACGCGCAGGTCCCCGGACGTGCT | |
| | GAAGGACTTCGCCCCCGGCTCGCAGCTGCCCATCCTGCTCTATGACAGCGACGCCAAG | |
| | ACAGACACGCTGCAGATCGAGGACTTTCTGGAGGAGACGCTGGGGCCGCCCGAGGAGT | |
| | CCAACACCGCCGGCAACGACGTTTTCCACAAGTTCTCCGCGTTCATCAAGAACCCGGT | |
| | GCCCGCGCAGGACGAAGCCCTGTACCAGCAGCTGCTGCGCGCCCTCGCCAGGCTGGAC | |
| | AGCTACCTGCGCGCGCCCCTGGAGCACGAGCTGGCGGGGGAGCCGCAGCTGCGCGAGT | |
| | CCCGCCGCCGCTTCCTGGACGGCGACAGGCTCACGCTGGCCGACTGCAGCCTCCTGCC | |
| | CAAGCTGCACATCGTCGACACGGTGTGCGCGCACTTCCGCCAGGCGCCCATCCCCGCG | |
| | GAGTGCGCGGCGTACGCCGTTACCTGGACAGCGCGATGCAGGAGGAGTTCATA | |
| | CACGTGTCCGCACAGCGCCGAGATCCTGGCGGCCTACCGGCCCGCCGTGCACCCCACG | |
| | CTAGCGCCCCACCCCGCGTCTGTCGCCCAATAAAGGCATCTTTGTCGGG | |
| | ORF Start: ATG at 17 | ORF Stop: TAG at 698 |

| | | |
|---|---|---|
| SEQ ID NO:64 | 227 aa | MW at 25431.7kD |

| | |
|---|---|
| NOV16e, | MAETKLQLFVKASEDGESVGHCPSCQRLFMVLLLKGVPFTLTTVDTRRSPDVLKDFAP |
| CG158983-01 | |
| Protein Sequence | GSQLPILLYDSDAKTDTLQIEDFLEETLGPPEESNTAGNDVFHKFSAFIHQPVPAQDE |
| | ALYQQLLRALARLDSYLRAPLEHELAGEPQLRESRRRFLDGDRLTLAQCSLLPKLHIV |
| | DTVCAHFRQAPIPAECAAYAVTWTARCRRKSSNTRVRTAPRSWRPTGPPCTPR |

Sequence comparison of the above protein sequences yields the following sequence relationships shown in Table 16B.

TABLE 16B
Comparison of NOV16a against NOV16b through NOV16e.

| | | Identities/ |
|---|---|---|
| | | Similarities for |
| Protein | NOV16a Residues/ | the Matched |
| Sequence | Match Residues | Region |
| Nov16b | 1 . . . 189 | 189/189 (100%) |
| | 1 . . . 189 | 189/189 (100%) |
| NOV16c | 1 . . . 189 | 189/189 (100%) |
| | 1 . . . 189 | 189/189 (100%) |
| Nov16d | 1 . . . 227 | 227/227 (100%) |
| | 1 . . . 227 | 227/227 (100%) |
| NOV16e | 1 . . . 227 | 227/227 (100%) |
| | 1 . . . 227 | 227/227 (100%) |

Further analysis of the NOV16a protein yielded the following properties shown in Table 16C.

TABLE 16C
Protein Sequence Properties NOV16a

| | |
|---|---|
| PSort | 0.9000 probability located in Golgi body; |
| analysis: | 0.7900 probability located in plasma membrane; |
| | 0.3000 probability located in microbody |
| | (peroxisome); 0.2000 probability located in |
| | endoplasmic reticulum (membrane) |
| SignalP | Cleavage site between residues 43 and 44 |
| analysis: | |

A search of the NOV16a protein against the Geneseq database, a proprietary database that contains sequences published in patents and patent publication, yielded several homologous proteins shown in Table 16D.

TABLE 16D
Geneseq Results for NOV16a

| | | NOV16a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAW61550 | Human chloride channel | 1 . . . 227 | 226/236 (95%) | e−129 |
| | protein - Homo sapiens, 241 | 6 . . . 241 | 227/236 (95%) | |
| | aa. [WO9830691-A1, 16 JUL. | | | |
| | 1998] | | | |
| AAU23722 | Novel human enzyme | 20 . . . 189 | 162/179 (90%) | 8e−87 |
| | polypeptide #808 - Homo | 6 . . . 184 | 163/179 (90%) | |
| | sapiens, 222 aa. | | | |
| | [WO200155301-A2, 02 AUG. | | | |
| | 2001] | | | |
| AAM40512 | Human polypeptide SEQ ID NO | 3 . . . 189 | 101/198 (51%) | 6e−49 |
| | 5443 - Homo sapiens, 312 aa. | 60 . . . 257 | 134/198 (67%) | |
| | [WO200153312-A1, 26 JUL. | | | |
| | 2001] | | | |
| AAM38726 | Human polypeptide SEQ ID NO | 3 . . . 189 | 101/198 (51%) | 6e−49 |
| | 1871 - Homo sapiens, 308 aa. | 71 . . . 268 | 134/198 (67%) | |
| | [WO200153312-A1, 26 JUL. | | | |
| | 2001] | | | |
| AAM79354 | Human protein SEQ ID NO | 3 . . . 189 | 101/198 (51%) | 6e−49 |
| | 3000 - Homo sapiens, 312 aa. | 60 . . . 257 | 134/198 (67%) | |
| | [WO200157190-A2, 09 AUG. | | | |
| | 2001] | | | |

In a BLAST search of public sequence datbases, the NOV16a protein was found to have homology to the proteins shown in the BLASTP data in Table 16E.

TABLE 16E
Public BLASTP Results for NOV16a

| | | NOV16a | Identities/ | |
|---|---|---|---|---|
| Protein | | Residues/ | Similarities for | |
| Accession | | Match | the Matched | Expect |
| Number | Protein/Organism/Length | Residues | Portion | Value |
| O95833 | Chloride intracellular | 30 . . . 189 | 159/169 (94%) | 4e−85 |
| | channel protein 3 - Homo | 1 . . . 169 | 160/169 (94%) | |
| | sapiens (Human), 207 aa. | | | |
| Q9D7P7 | 2300003G24Rik protein - Mus | 30 . . . 189 | 143/169 (84%) | 2e−76 |
| | musculus (Mouse), 207 aa. | 1 . . . 169 | 149/169 (87%) | |
| Q9Z0W7 | Chloride intracellular | 3 . . . 187 | 102/196 (52%) | 3e−49 |
| | channel protein 4 | 16 . . . 211 | 133/196 (67%) | |
| | (Intracellular chloride ion | | | |
| | channel protein P64H1) - | | | |
| | Rattus norvegicus (Rat), 253 | | | |
| | aa. | | | |
| Q9QYB1 | Intracellular chloride | 3 . . . 187 | 102/196 (52%) | 5e−49 |
| | channel protein - Mus | 16 . . . 211 | 133/196 (67%) | |
| | musculus (Mouse), 253 aa. | | | |
| Q9Y696 | Chloride intracellular | 3 . . . 189 | 101/198 (51%) | 2e−48 |
| | channel protein 4 | 16 . . . 213 | 134/198 (67%) | |
| | (Intracellular chloride ion | | | |
| | channel protein p64H1) - Homo | | | |
| | sapiens (Human), 253 aa. | | | |

PFam analysis predicts that the NOV16a protein contains the domains shown in the Table 16F.

TABLE 16F
Domain Analysis of NOV16a

| | | | |
|---|---|---|---|
| | | Identities/ | |
| | | Similarities for | |
| Pfam | NOV16a Match | the Matched | Expect |
| Domain | Region | Region | Value |

No Significant Matches Found

### Example 17

The NOV17 clone was analyzed, and the nucleotide and encoded polypeptide sequences are shown in Table 17A.

TABLE 17A
NOV17 Sequence Analysis

| | |
|---|---|
| SEQ ID NO: 65 | 2400 bp |

| NOV17a, | GTGCGCGTTGGGGCGGCCGGCCAATGCCGGACCGCTTCGGCACCCCCCGCCCGATCCC | |
|---|---|---|
| CG159015-01 DNA | | |
| Sequence | TCCACCCGTGGGCCGGCAATGGCGGGCGCAGTTTCGCTCTTGGGTGTGGTGGGGCTGC | |
| | TGCTTGTGTCTCCGCTGTCCGGCGTCCTAGGAGACCGCGCCAATCCCGACCTCCGGGC | |
| | ACACCCAGGGAACGCAGCCCACCCCGGCTCTGGAGCCACGGAACCCCGGCGGCGACCA | |
| | CCGCTCAAGGATCAACGCGAGCGGACCCGGGCCGGGTCGCTGCCTCTGGGGGCGCTGT | |
| | ACACCGCGGCCGTCGCGGCTTTTGTGCTGTACAAGTGTTTGCAGGGGAAAGATGAAAC | |
| | TGCGGTTCTCCACGAGGAGGCAAGCAAGCAGCAGCCACTGCAGTCAGAGCAACAGCTG | |
| | GCCCAGTTGACACAACAGCTGGCCCAGACAGAGCAGCACCTGAACAACCTGATGGCCC | |
| | AGCTGGACCCCCTTTTTGAGCGTGTGACTACTCTGGCTGGAGCCCAGCAGGAGCTTCT | |
| | GAACATGAAGCTATGGACCATCCACGAGCTGCTGCAAGATAGCAAGCCGGACAAGGAT | |
| | ATCGACGCTTCAGAACCAGGTGAACGCTCGGGAGGCGAGTCTCCTGGAGGTCGAGACA | |
| | AAGTCTCTGAAACTGGAACATTCCTGATCTCTCCCCACACAGAGGCCAGCAGACCTCT | |
| | TCCTGAGGACTTCTGTTTAAAGGAGGACGAGGAGGAGGTTGGTGACAGTCAGCCCTGG | |
| | GAGGAGCCCACAAACTGGAGCACAGAGACATGGAACCTAGCTACTTCCTGGGAGGTGG | |
| | GGCGGGGACTACGGAGAAGGTCCAGCCAGGCTGTGGCAAAGCGCCCCAGTCACAGCCT | |
| | TGGCTGGGAAGGAGGGACGACAGCTGAAGGTCGACTAAAACAAAGTCTGTTTTCATGA | |
| | TGGAGTGCTCCTGTGTGTTTTTTCGATCCTAGTTGGTTGTACACACCCATACTAGGTG | |
| | CCTAAGGACAACTGGGCCTTCTTGAAGAGCTGTCCTTATTAGGACAAAAAGAGGCTGC | |
| | CTTCCAGTGTGACAGCAGAGAAGATAGAGGGAGCTCCAGCTCTTTTCCTCGTATTCCT | |
| | GAGGCCACCAGCATGCCCGCGTTCAGGGCCCAAAAATCCCTTTTCTCATAGCAAAACT | |
| | GAGACAGAAGGGTCTTTCCCAAAAAAAAGAAAAAAAAACTTTACTCAAATCCAGTGGA | |
| | AAAATAAATCATACAAACTATACACAACATAAAAATAGCCACATTTACAAACCTCCAC | |
| | CCTTGATAAATGACGGGCCATGCACACACCACAGAGCTTATCAGTCCCAAATCCCCTC | |
| | ATCTGTGTTAGGGGCTGGTTCATTTGAGGTTTAGTTGGGTTGGACTTGGTTTCCTGAT | |
| | TCTTCTTTTTTAATAAAATTTCTTAATTATTTTTTCTTAAATAGACACAGGGTCTCAC | |
| | TCACTGTGTTGCCCAGGCTGGTCTTGAACTCCTGGGCTGGAATGATCCTGCCACCTCT | |
| | GCTTCCCAAAGTGCTGGGATTACAGGCATGAGCCACTGTGCCTGGCCGTGATTTTTAA | |
| | GAGTTGGTCAGATGATCTGGAGTAGCTTGGTCCAGGCAAACAGAAAGTGACCTTTGTC | |
| | AAATCATGAAGGGTTCTGTTTTGTTCAGTACTGAAGATTCCTTTGTACTCTTGGCTGT | |
| | GACCTATCCCTGAGGTATCCTGAGTTCTGGAATCTATAAGATTCCTCTAGTTTTTCTG | |
| | GCTGCTGATAGCCCAAGTCAGACTGTGGTACCAGCGTGACAGCTCCTCCTGGTCTGTG | |
| | CACATAAGCAGTAGCTTCTCATGAGGGAAGGACAGGTGTGAGCTGTTGATGGTCAGGG | |
| | CTGTTGGGACCTGTGTTTTCAGCCAAAGCTACGACGAGATTCTCATACTGCTGGAGCC | |
| | GTTGCAGAGGCAGAGOGAGCAGGTCCTGGAGCTGAAGCCCCCCAAACCCAGGGCGGCC | |
| | TTCCTGAAGCCCTACAAACCTCCGGAAACCTTTATTTTTCTTTAGCTGCTCCTGCAGG | |
| | GTGGTCTGGGACCTCTCTGAGTTGGCAGCAAATTGGTTATAGAGCTCCAAGTGGCGGC | |
| | AGAAGCCCTCCAGCCCTTGGCCCCAGCATCCTCCTTCCAGGTAGGGAAGCAGCTCCTG | |
| | GCTGGCGCCGTAGATGAGCTCCCAGGAGCCAAACAGGGCCTGGCGCTCAGGTGGTCGC | |
| | AGGGTCCCCTTGGCTTTCAGGATCCCCAAAAAGTACGTGGCCACCAGCCCCAGCTGTT | |
| | CTTGGTAGCGCCGCTCGGTCTCTAGCAGCTCCCGGGCGGTGCAGGCGCGTTTCCGCTC | |
| | CCAGCGGGCACGCTGCTCTTGCACCGGGCACCGCGAACCGGGGCAUGAGAGCTCCATG | |
| | CCCTGGCTGAGGGATCGACACT | |
| | ORF Start: ATG at 77 | ORF Stop: TGA at 926 |

| | | |
|---|---|---|
| SEQ ID NO: 66 | 283 aa | MW at 30494.7kD |

| NOV17a, | MAGAVSLLGVVGLLLVSALSGVLGDRANPDLRAHPGNAAHPGSGATEPRRRPPLKDQR | |
|---|---|---|
| CG159015-01 | | |
| Protein Sequence | ERTRAGSLPLGALYTAAVAAFVLYKCLQGKDETAVLHEEASKQQPLQSEQQLAQLTQQ | |
| | LAQTEQHLNNLMAQLDPLFERVTTLAGAQQELLNMKLWTTHELLQDSKPDKDMEASEP | |
| | GEGSGGESAGGGDKVSETGTFLISPHTEASRPLPEDFCLKEDEEEVCDSQAWEEPTNW | |
| | STETWNLATSWEVGRGLRRRCSQAVAKGPSHSLGWEGGTTAEGRLKQSLFS | |
| | SEQ ID NO: 67 | 1449 bp |

| NOV17b, | GGTGAGAAGTTGGTGGCGTGAGATTAAAAAAACCGTTTTCGGGCATAACTTTCTAAG | |
|---|---|---|
| CG159015-02 DNA | | |
| Sequence | ACTATAGGCTTTCAGAGGCATTGTGGCTAGCAGAATAGCTAATAGACACGAAATGAAC | |
| | AAATACAGGAAAGCTAGAATGACACTATCTTATGCAAATATGGTCTGGCCCCGCCCTA | |
| | CGGGGAGTGGGCGTGGCCTCCCCGGAGCCGGCCGGCCTGCTCGCGTGCOCGTGCGCGT | |
| | TGGGGCGGCCGGCCAATGCCGGACCGCTTCCGCACCGCCCGCCCGATCCCTCCACCCG | |
| | TGGGCCGGCAATGGCGGGCGCAGTTTCGCTCTTGGGTGTGGTGGGGCTGCTGCTTGTG | |
| | TCTGCGCTGTCCGGGGTCCTAGGAGACCGCGCCAATCCCGACCTCCGGGCACACCCAG | |
| | GTAACGCAGCCCACCCCGGCTCTGGAGCCACGGAACCCCGGCGGCGACCACCGCTCAG | |
| | GGATCAACGCGAGCGGACCCGGGCCGGGTCGCTGCCTCTGGGGGCGCTGTACACCGCG | |
| | GCCGTCGCGGCTTTTGTGCTGTACAAGTGTTTGCACGGGAAAGATGGTGCTGCGGTTC | |
| | TCCACGAGGAGGCAAGCAAGCAGCAGCCACTGCAGTCAGAGCGCCAGCTGCCCCAGTT | |
| | GACACAACAGCTGGCCCAGACAGAGCAGCACCTGAACAACCTGATGGCCCAGCTGGAC | |
| | GCCCTTTTTGAGCGGGTGACTACTCTGGCTGGACCCCAGCAGGAGCTTCTGAACATGA | |
| | AGCTATGGACCATCCACGAGCTGCTGCAAGATAGCAAGCCGGACGAGGATATGGAGGC | |
| | TTCAGAACCAGGTGAAGCCTCGGGAGGCGAGTCTGCTGGAGGTGGAGACATCGTCTCT | |
| | GAAACTGGAACATTCCTGATCTCTCCCCACACAGAGGCCAGCAGACCTCTTCCTGAGG | |
| | ACTTCTGTTTAAAGGAGGACGAGGAGGAGATTGGTGACAGTCACGCCTGGGAGGAGCC | |
| | CACAAACTGGAGCACAGAGACATGGAACCTAGCTACTTCCTGGGAGGTGGGGCGGGGA | |
| | CTACGGAGAAGGTGCAGCCAGGCTGTGGCAAAGGGCCCCAGTCACAGCCTTCGCTGGG | |
| | AAGGAGGGACGACAGCTGAAGGTCGACTAAAACAAAGTCTGTTTTCATGATGGAGTGC | |
| | TCCTGTGTGTTTTTTCGATCCTAGTTGGTTGTACACACCCATACTAGGTGCCTCTGGA | |
| | CAACTGGGCCTTCTTGAAGAGCTGTCCTTATTAGGACAAAAAGAGGCTGCCTTCCAGT | |
| | GTGACAGCAGAGAAGATAGAGGGAGCTCCAGCTCTTTTCCTCGTATTCCTGAGGCCAC | |
| | CAGCATGCCCGCGTTCAGGGCCCAAAAATCCCTTTTCTCATAGCGCATCTGAGACAGA | |
| | AGGGTCTTTCCCAAAAAAAAGAAAAAAAACTTTACTCAAATCCAGTGGAAAAATAAA | |
| | ORF Start: ATG at 148 | ORF Stop: TGA at 1150 |

| | | |
|---|---|---|
| SEQ ID NO: 68 | 334 aa | MW at 35589.5kD |

| NOV17b, | MQIWSGPALRGVGVASPEPAGLLACACALGRPANAGPLRHRPPDPSTRCPEQAGAVSL | |
|---|---|---|
| CG159015-02 | | |
| Protein Sequence | LGVVGLLLVSALSGVLGDRANPDLRAHPGNAAHPGSGATEPRRRPPLKDQRGERTKGS | |
| | LPLGALYTAAVAAFVLYKCLQGKDETAVLHEEASKQQPLQSEQQLAQLTQLAQQTEQH | |
| | LNNLMAQLDPLFERVTTLAGAQQELLNMKLWTIHELLQDSKPDKDMEASEPGEGSGGE | |
| | SAGGGDKVSETCTFLISPHTEASRPLPEDFCLKEDEEEIGDSQAWEEPTNWSTETWNL | |
| | ATSWEVGRGLRRRCSQAVAKGPSHSLGWEGGTTAEGRLKQSLFS | |
| | SEQ ID NO: 69 | 539 bp |

| NOV17c, | CCGGCCAATGCCGGACCGCTTCCGCACCGCCCGCCCGATCCCTCCACCCGTGGGCCGG | |
|---|---|---|
| CG159015-03 | DNA | |
| Sequence | CAATGGCGGGCGCAGTTTCGCTCTTGGGTGTGGTGGGGCTGCTGCTTGTGTCTGCGCT | |
| | GTCCGGGGTCCTAGGAGACCCCGCCAATCCCGACCTCCGGGCACACCCAGGGGACGCA | |
| | GCCCACCCCGGCTCTGGAGCCACGGGTCCCCGGCGGCGACCACCGCTCGTGGATCAAC | |
| | GCGAGCGGACCCGGGCCGGGTCGCTGCCTCTGGGCGCGCTGTACACCGCGGCCGTCGC | |
| | GGCTTTTGTGCTGTACAAGTGTTTGCACGGGACAGATGAAACTGCGGTTCTCCACGAG | |
| | GAGGCAAGCAAGCAGCAGCCACTGCAGTCAGAGCAACAGCTGCCCCAGTTGACACAAC | |
| | AGCTGGCCCAGACAGAGCAGCACCTGAACAACCTGATGCCCCAGCTGGACCCCCTTTT | |
| | TGAGCGCCCAGCAGGAGCTTCTGAACATGAAGCTATGGACCATCCACGAGCTGCTGCA | |
| | AGATAGCAAGCCCGGAC | |
| | ORF Start: ATG at 61 | ORF Stop: TAG at 526 |

| | | |
|---|---|---|
| SEQ ID NO: 70 | 155 aa | MW at 16521.5kD |

| NOV17c, | MAGAVSLLGVVGLLLVSALSGVLGDRANPDLRAHPGNAAHPGSGATEPRRRPPLKDQR | |
|---|---|---|
| CG159015-03 | | |
| Protein Sequence | ERTRAGSLPLGALYTAAVAAFVLYKCLQGKDETAVLHEEASKQQPLQSEQQLAQLTQQ | |
| | LAQTEQHLNNLMAQLDPLFERPAGASEHEAMDHPRAAAR | |
| | SEQ ID NO: 71 | 774 bp |

| NOV17d, | GTGCGCGTTGGGGCGGCCGGCCAATGCCGGACCGCTTCGGCACCGCCCGCCCGATCCC | |
|---|---|---|
| CG159015-04 DNA | | |
| Sequence | TCCACCCGTGGGCCGGCAATGGCGGGCGCAGTTTCGCTCTTGGGTGTGGTGGOGCTGC | |
| | TGCTTGTGTCTGCGCTGTCCGGGGTCCTAGGAGACCGCGCCAATCCCGACCTCCOGGC | |
| | ACACCCAGGGAACGCAGCCCACCCCGGCTCTGGAGCCACGGAACCCCGGCGGCGACCA | |
| | CCGCTCAAGGATCAACGCGAGCGGACCCGGGCCGGGTCGCTGCCTCTGGGGGCGCTGT | |
| | ACACCGCGGCCGTCGCGGCTTTTGTGCTGTACAAGTGTTTGCAGGOGAAAGATGAAAC | |
| | TGCGGTTCTCCACGAGGAGGCAAGCAAGCAGCAGCCACTGCAOTCAGAGCAACAGCTG | |
| | GCCCAGTTGACACAACAGCTGGCCCAGACAGAGCAGCACCTGAACAACCTCATGGCCC | |
| | AGCTGGACCCCCTTTTTGAGCGGTGAGGAGAGCAATOATTCTGTGAATTTTTGGGGAA | |
| | TTTGTGGCAGGAGGGAGGAATGGGGACATAUGTTGGGAGCCACTGAGTGGACATTTCT | |
| | TCAGTGTGACTACTCTGGCTGGAGCCCAGCAGGAGCTTCTGAACATGAAGCTATGGAC | |
| | CATCCACGAGCTGCTGCAAGATAGCAAGCCGGACAAGGATATGGAGGCTTCAGAACCA | |
| | GGTGAAGGCTCGGGAGGCGAGTCTGCTGGAGGTGGAGACAAAGTCTCTOAAACTGGAA | |
| | CATTCCTGATCTCTCCCCCA | |
| | ORF Start: ATG at 77 | ORF Stop: TGA at 488 |

| | | |
|---|---|---|
| SEQ ID NO: 72 | 137 aa | MW at 14665.5kD |

| | |
|---|---|
| NOV17d, | MAGAVSLLGVVGLLLVSALSGVLGDRANPDLRANPGNAAHPGSGATEPRRRPPLKDQR |
| CG159015-04 | |
| Protein Sequence | ERTRACSLPLGALYTAAVAAFVLYKCLQGKDETAVLHEEASKQQPLQSEQQLAQLTQQ |
| | LAQTEQHLNNLMAQLDPLFER |

Sequence comparison of the above protein sequences yields the following sequence relationships shown in Table 17B.

TABLE 17B
Comparison of NOV17a against NOV17b through NOV17d.

| | | Identities/ |
|---|---|---|
| | | Similarities for |
| Protein | NOV17a Residues/ | the Matched |
| Sequence | Match Residues | Region |
| NOV17b | 1 . . . 283 | 282/283 (99%) |
| | 52 . . . 334 | 283/283 (99%) |
| NOV17c | 1 . . . 137 | 137/137 (100%) |
| | 1 . . . 137 | 137/137 (100%) |
| NOV17d | 1 . . . 137 | 137/137 (100%) |
| | 1 . . . 137 | 137/137 (100%) |

Further analysis of the NOV17a protein yielded the following properties shown in Table 17C.

TABLE 17C
Protein Sequence Properties NOV17a

| | |
|---|---|
| PSort | 0.8200 probability located in outside; 0.1000 |
| analysis: | probability located in endoplasmic reticulum |
| | (membrane); 0.1000 probability located in |
| | endoplasmic reticulum (lumen); 0.1000 |
| | probability located in lysosome (lumen) |
| SignalP | Cleavage site between residues 25 and 26 |
| analysis: | |

A search of the NOV17a protein against the Geneseq database, a proprietary database that contains sequences published in patents and patent publication, yielded several homologous proteins shown in Table 17D.

TABLE 17D
Geneseq Results for NOV17a

| | | NOV17a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB72305 | Rat protein isolated from | 1 . . . 262 | 163/263 (61%) | 1e−79 |
| | skin cells SEQ ID NO: 629 - | 1 . . . 233 | 184/263 (68%) | |
| | Rattus sp, 242 aa. | | | |
| | [WO200190357-A1, 29 NOV. | | | |
| | 2001] | | | |
| AAB88440 | Human membrane or secretory | 1 . . . 137 | 137/137 (100%) | 2e−72 |
| | protein clone PSEC0222 - | 1 . . . 137 | 137/137 (100%) | |
| | Homo sapiens, 139 aa. | | | |
| | [EP1067182-A2, 10 JAN. 2001] | | | |
| ABB68896 | Drosophila melanogaster | 85 . . . 224 | 33/140 (23%) | 0.001 |
| | polypeptide SEQ ID NO | 816 . . . 943 | 54/140 (38%) | |
| | 33480 - Drosophila | | | |
| | melanogaster, 2439 aa. | | | |
| | [WO200171042-A2, | | | |
| | 27 SEP. 2001] | | | |
| ABG28274 | Novel human diagnostic | 136 . . . 269 | 34/140 (24%) | 0.47 |
| | protein #28265 - Homo | 283 . . . 413 | 57/140 (40%) | |
| | sapiens, 1121 aa. | | | |
| | [WO200175067-A2, 11 OCT. | | | |
| | 2001] | | | |
| ABB64814 | Drosophila melanogaster | 59 . . . 172 | 29/120 (24%) | 0.81 |
| | polypeptide SEQ ID NO | 2621 . . . 2731 | 54/120 (44%) | |
| | 21234 - Drosophila | | | |
| | melanogaster, 3583 aa. | | | |
| | [WO200171042-A2, | | | |
| | 27 SEP. 2001] | | | |

In a BLAST search of public sequence datbases, the NOV17a protein was found to have homology to the proteins shown in the BLASTP data in Table 17E.

TABLE 17E
Public BLASTP Results for NOV17a

| | | NOV17a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8WV48 | Similar to RIKEN cDNA | 1 . . . 283 | 283/283 (100%) | e−163 |
| | 1110032022 gene - Homo | 1 . . . 283 | 283/283 (100%) | |
| | sapiens (Human), 283 aa. | | | |
| Q9DCC3 | 1110032022Rik protein | 1 . . . 262 | 153/262 (58%) | 1e−74 |
| | (Hypothetical 26.6 kDa | 1 . . . 233 | 178/262 (67%) | |
| | protein) - Mus musculus | | | |
| | (Mouse), 242 aa. | | | |
| CAC39804 | Sequence 247 from Patent | 1 . . . 137 | 137/137 (100%) | 5e−72 |
| | EP1067182 - Homo sapiens | 1 . . . 137 | 137/137 (100%) | |
| | (Human), 139 aa. | | | |
| Q9CTB6 | 1110032022Rik protein - Mus | 35 . . . 262 | 133/228 (58%) | 4e−64 |
| | musculus (Mouse), 259 aa | 52 . . . 250 | 153/228 (66%) | |
| | (fragment). | | | |
| Q9VMS2 | CG14023 protein - Drosophila | 85 . . . 224 | 33/140 (23%) | 0.004 |
| | melanogaster (Fruit fly), | 816 . . . 943 | 54/140 (38%) | |
| | 2439 aa. | | | |

PFam analysis predicts that the NOV17a protein contains the domains shown in the Table 17F.

TABLE 17F
Domain Analysis of NOV17a

| | | | |
|---|---|---|---|
| | | Identities/ | |
| | | Similarities for | |
| Pfam | NOV17a Match | the Matched | Expect |
| Domain | Region | Region | Value |

No Significant Matches Found

### Example 18

The NOV18 clone was analyzed, and the nucleotide and encoded polypeptide sequences are shown in Table 18A.

TABLE 18A
NOV18 Sequence Analysis

| | |
|---|---|
| SEQ ID NO: 73 | 2463 bp |

| NOV18a, | AACTCTCCTATTCATGGAGGCGACACTGAGGATGCTTTCCACATGAACCCTGAAGTG | |
|---|---|---|
| CG173007-01 DNA | | |
| Sequence | AACTTCTGATACATTTCCTGCAGCAAGAGAAGGCAGCCAACATGAAGGAAAATGTGGC | |
| | ATCTGCAACCGTTTTCACTCTGCTACTTTTTCTCGCACCTGCCTTCTGATGGACAG | |
| | TTACCTCCTGGAAAACCTGAGATCTTTAAATGTCGTTCTCCCAATAAGGAAACATTCA | |
| | CCTGCTGGTGGAGGCCTGGGACAGATGGAGGACTTCCTACCAGCTCCTGCCACTTTGG | |
| | CAAGCAGTACACCTCCATGTGGAGGACATACATCATGATGGTCAATGCCACTAACCAG | |
| | ATGGGAAGCAGTTTCTCGGATGAACTTTATGTGGACGTGACTTACATAGTTCAGCCAG | |
| | ACCCTCCTTTGGAGCTGGCTGTGGAAGTAAAACAGCCAGAAGACAGAAAACCCTACCT | |
| | GTGGATTAAATGGTCTCCACCTACCCTGATTGACTTAAAAACTGGTTGGTTCACGCTC | |
| | CTGTATGAAATTCGATTAAAACCCGAGAAAGCAGCTGAGTGGGAGATCCATTTTGCTG | |
| | GGCAGCAAACAGAGTTTAAGATTCTCAGCCTACATCCAGGACAGAAATACCTTGTCCA | |
| | GGTTCGCTGCAAACCAGACCATGGATACTGGAGTGCATGGAGTCCAGCGACCTTCATT | |
| | CAGATACCTAGTGACTTCACCATGAATGATACAACCGTGTGGATCTCTGTGGCTGTCC | |
| | TTTCTGCTGTCATCTGTTTGATTATTGTCTGGGCAGTGGCTTTGAAGGGCTATAGCAT | |
| | GGTGACCTGCATCTTTCCGCCAGTTCCTGGGCCAAAAATAAAAGGATTTGATGCTCAT | |
| | CTGTTGGAGAAGGGCAAGTCTGAAGAACTACTGAGTGCCTTCGGATGCCGTGACTTTC | |
| | CTCCCACTTCTGACTATGAGGACTTGCTGGTGGAGTATTTAGAAGTAGATGATAGTGA | |
| | GGACCAGCATCTAATGTCAGTCCATTCAAAGAACACCCAATGTCGGGTATCTGAACCC | |
| | ACATACCTGGATCCTGACACTGACTCAGGCCGGGGGAGCTGTGACAGCCCTTCCCTTT | |
| | TGTCTGAAAAGTGTGAGGAACCCCAGGCCAATCCCTCCACATTCTATGATCCTGAGGT | |
| | CATTGAGAAGCCAGAGAATCCTGAAACAACCCACACCTGGGACCCCCAGTGCATAAGC | |
| | ATGGAAGGCAAAATCCCCTATTTTCATGCTGGTGGATCCAAATGTTCAACATGGCCCT | |
| | TACCACAGCCCAGCCAGCACAACCCCAGATCCTCTTACCACAATATTACTGATGTGTG | |
| | TGAGCTGGCTGTGGGCCCTGCAGGTGCACCGGCCACTCTGTTGAATGAAGCAGGTAAA | |
| | GATGCTTTAAAATCCTCTCAAACCATTAAGTCTACAGAAGAGGGAAAGGCAACCCACC | |
| | AGAGGGAGGTAGAAAGCTTCCATTCTGAGACTGACCACCATACGCCCTGGCTGCTGCC | |
| | CCAGGAGAAAACCCCCTTTGGCTCCGCTAAACCCTTGCATTATGTGGAGATTCACAAG | |
| | GTCAACAAAGATGGTCCATTATCATTGCTACCAAAACAGAGAGAGAACAGCGGCAAGC | |
| | CCAAGAAGCCCCGGACTCCTCAGAACAATAAGGAGTATGCCAAGGTGTCCGGGGTCAT | |
| | CGATAACAACATCCTGGTGTTGGTGCCAGATCCACATGCTAAAAACGTGGCTTGCTTT | |
| | GAAGAATCAGCCAAAGAGGCCCCACCATCACTTGAACAGAATCAAGCTGAGAAAGCCC | |
| | TGGCCAACTTCACTGCAACATCAAGCAAGTGCAGGCTCCAGCTGGGTGGTTTGGATTA | |
| | CCTGGATCCCGCATGTTTTACACACTCCTTTCACTGATAGCTTGACTAATCGAATGAT | |
| | TGGTTAAAATGTGATTTTTCTTCAGGTAACACTACAGAGTACGTGAAATGCTCAAGAA | |
| | TGTAGTCAGACTGACACTACTAAAGCTCCCAGCTCCTTTCATGCTCCATTTTTAACCA | |
| | CTTGCCTCTTTCTCCAGCAGCTGATTCCAGAACAAATCATTATGTTTCCTAACTGTGA | |
| | TTTGTAGATTTACTTTTTGCTGTTAGTTATAAAACTATGTGTTCAATGAAATAAAAGC | |
| | ACACTGCTTAGTATTCTTGAGGGACAATGCCAATAGGTATATCCTCTGGAAAAGGCTT | |
| | TCATCATTTGGCATGGGACAGACGGAAATGAAATTGTCAAAATTGTTTACCATAGAAA | |
| | GATGACAAAAGAAAATTTTCCACATAGGAAAATGCCATGAAAATTGCTTTTGAAAAAC | |
| | AACTGCATAACCTTTACACTCCTCGTCCATTTTATTACGATTACCCAAATATAACCAT | |
| | TTAAAGAAAGAATGCATTCCAGAACAAATTGTTTACATAAGTTCCTATACCTTACTGA | |
| | CACATTGCTGATATGCAAGTAAGAAAT | |
| | ORF Start: ATG at 100 | ORF Stop: TGA at 1891 |

| | | |
|---|---|---|
| SEQ ID NO: 74 | 597 aa | MW at 66638.8kD |

| | |
|---|---|
| NOV18a, | MKENVASATVFTLLLFLNTCLLNGQLPPGKPEIFKCRSPNKETFTCWWRPGTDGGLPT |
| CG173007-01 | |
| Protein Sequence | NSCHFGKQYTSMWRTYIMMVNATNQMGSSFSDELYVDVTYIVQPDPPLELAVEVKQPE |
| | DRKPYLWIKWSPPTLIDLKTGWFTLLYEIRLKPEKAAEWEIHFAGQQTEFKILSLHPG |
| | QKYLVQVRCKPDHGYWSAWSPATFIQIPSDFTMNDTTVWISVAVLSAVICLIIVWAVA |
| | LKGYSMVTCIPPPVPGPKIKGFDAHLLEKGKSEELLSALGCQDFPPTSDYEDLLVEYL |
| | EVDDSEDQHLMSVHSKEHPSQGMKPTYLDPDTDSGRGSCDSPSLLSEKCEEPQANPST |
| | FYDPEVIEKPENPETTHTWDPQCISMEGKIPYFHAGGSKCSTWPLPQPSQHNPRSSYH |
| | NITDVCELAVGPAGAPATLLNEAGKDALKSSQTIKSREEGKATQQREVESFHSETDQD |
| | TPWLLPQEKTPFGSAKPLDYVEIHKVNKDGALSLLPKQRENSGKPKKPGTPENNKEYA |
| | KVSGVMDNNILVLVPDPHAKNVACFEESAKEAPPSLEQNQAEKALANFTATSSKCRLQ |
| | LGGLDYLDPACFTHSFH |

Further analysis of the NOV18a protein yielded the following properties shown in Table 18B.

TABLE 18B
Protein Sequence Properties NOV18a

| | |
|---|---|
| PSort | 0.4600 probability located in plasma membrane; |
| analysis: | 0.1447 probability located in microbody |
| | (peroxisome); 0.1000 probability located in |
| | endoplasmic reticulum (membrane); 0.1000 |
| | probability located in endoplasmic reticulum (lumen) |
| SignalP | Cleavage site between residues 25 and 26 |
| analysis: | |

A search of the NOV18a protein against the Geneseq database, a proprietary database that contains sequences published in patents and patent publication, yielded several homologous proteins shown in Table 18C.

TABLE 18C
Geneseq Results for NOV18a

| | | NOV18a | Identities/ | |
|---|---|---|---|---|
| | | Residues/ | Similarities for | |
| Geneseq | Protein/Organism/Length | Match | the Hatched | Expect |
| Identifier | [Patent #, Date] | Residues | Region | Value |
| AAU99354 | Human prolactin receptor | 1 . . . 597 | 597/622 (95%) | 0.0 |
| | (PRLR) protein - Homo | 1 . . . 622 | 597/622 (95%) | |
| | sapiens, 622 aa. | | | |
| | [WO200250098-A2, 27 JUN. | | | |
| | 2002] | | | |
| AAR10795 | Human prolactin receptor - | 1 . . . 597 | 597/622 (95%) | 0.0 |
| | Homo sapiens, 622 aa. | 1 . . . 622 | 597/622 (95%) | |
| | [US4992378-A, 12 FEB. 1991] | | | |
| AAU99355 | Human prolactin receptor | 1 . . . 597 | 596/622 (95%) | 0.0 |
| | (PRLR) variant protein - | 1 . . . 622 | 597/622 (95%) | |
| | Homo sapiens, 622 aa. | | | |
| | [WO200250098-A2, 27 JUN. | | | |
| | 2002] | | | |
| AAY95527 | Human prolactin receptor | 1 . . . 311 | 311/336 (92%) | 0.0 |
| | novel isoform - Homo | 1 . . . 336 | 311/336 (92%) | |
| | sapiens, 349 aa. [US6083753- | | | |
| | A, 04 JUL. 2000] | | | |
| AAY96921 | Soluble human prolactin | 1 . . . 311 | 311/336 (92%) | 0.0 |
| | receptor clone F - Homo | 1 . . . 336 | 311/336 (92%) | |
| | sapiens, 349 aa. [US6083714- | | | |
| | A, 04 JUL. 2000] | | | |

In a BLAST search of public sequence datbases, the NOV18a protein was found to have homology to the proteins shown in the BLASTP data in Table 18D.

TABLE 18D
Public BLASTP Results for NOV18a

| | | NOV18a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P16471 | Prolactin receptor precursor | 1 . . . 597 | 597/622 (95%) | 0.0 |
| | (PRL-R) - Homo sapiens | 1 . . . 622 | 597/622 (95%) | |
| | (Human), 622 aa. | | | |
| Q9N0J7 | Prolactin receptor | 1 . . . 597 | 531/622 (85%) | 0.0 |
| | precursor - Callithrix jacchus | 1 . . . 622 | 555/622 (88%) | |
| | (Common marmoset), 622 aa. | | | |
| P14787 | Prolactin receptor precursor | 1 . . . 597 | 450/624 (72%) | 0.0 |
| | (PRL-R) - Oryctolagus | 1 . . . 616 | 496/624 (79%) | |
| | cuniculus (Rabbit), 616 aa. | | | |
| Q9XS92 | Prolactin receptor | 1 . . . 597 | 407/625 (65%) | 0.0 |
| | precursor - Trichosurus | 1 . . . 625 | 476/625 (76%) | |
| | vulpecula (Brush-tailed | | | |
| | possum), 625 aa. | | | |
| A36116 | prolactin receptor 2 | 7 . . . 597 | 406/618 (65%) | 0.0 |
| | precursor - rat, 610 aa. | 3 . . . 610 | 472/618 (75%) | |

PFam analysis predicts that the NOV18a protein contains the domains shown in the Table 18E.

TABLE 18E
Domain Analysis of NOV18a

| | | Identities/ | |
|---|---|---|---|
| | | Similarities for | |
| Pfam | NOV18a Match | the Matched | Expect |
| Domain | Region | Region | Value |
| fn3 | 102 . . . 194 | 23/94 (24%) | 0.051 |
| | | 58/94 (62%) | |

### Example 19

The NOV19 clone was analyzed, and the nucleotide and encoded polypeptide sequences are shown in Table 19A.

TABLE 19A
NOV19 Sequence Analysis

| | |
|---|---|
| SEQ ID NO: 75 | 2221 bp |

| NOV19a, | AGCGGGCCGGGCGGCGGCGGGGAGATGCGGCTGCTGGCACTCGCGGCGGCCGCCCTGC | |
|---|---|---|
| CG173357-01 DNA | | |
| Sequence | TGGCGCGGGCTCCGGCTCCGGAGGTCTGTGCGGCCCTCTCTGTCACTGTGTCCCCGGG | |
| | GCCCGTGGTTGACTACCTGGAGGGGGAGAATGCCACTCTCCTCTGCCACGTCTCCCAG | |
| | AAAAGGCGGAAGGACAGCTTGCTGGCCGTGCGCTGGTTCTTTGCACACTCCTTCGACT | |
| | CCCAGGAGGCCTTGATGGTGAAGATGACCAAGCTCCGGGTGGTGCAGTACTATGGGAT | |
| | TTTCAGCCGCAGCGCCAAACGGCGGAGGCTCCGCCTGCTGGAGGAGCAGCGCGGGGCG | |
| | CTCTACAGGCTCTCCGTCTTGACACTGCACCCCTCCGATCGGGGCATTACGTCTGGCA | |
| | GAGTCCAGGAAATCAGCAGGCACAGGAACAAGTGGACGCCCTGGTCCGTTGGCTCCTC | |
| | AGCCACGGAATGAGAGTCATTTCCCTCAAGCTTCTGTAGGAGTCATCCTTTGAGAGAA | |
| | ACAAAAGAGACTTGGGCATTTTTTGAAGATCTCTATGTGTATGCTGTCCTCGTGTGCT | |
| | GCATGGGGATCCTCAGCATTCTGCTCTTCATGCTGGTCATCGTCTGGCAGTCTGTGTT | |
| | TAACAAGCGGAATCCAGAGTGAGACATTATTTGGTGTCATGCCCTCAGTATCAGCTCA | |
| | GGGGAGAGCTGTCACTAGCGTGACCAGCTTGGCCCCACTACAGCCCCAGGGAAGGGCG | |
| | AGGCAGAAGGAGAAGCCTGACATTCCTCCCGCAGTCCCTGCCAAAGCTCCGATACCCC | |
| | CCACGTTCCATAACCGAAGCTGCTGAACCACAGAGAAGGTGTCACGCTGCCAATCGAT | |
| | TGCTGAGGAAAACTTAACCTATGCCGAGCTGGAGCTGATCAGTCCCCACCGGGCTGCC | |
| | AAGGCGCCCCCACCAGCACTGTCTACGCCCAGATCCTCTTCGAGGAGAACGCAGCTGT | |
| | ACTACAGCGTCCACCTCCAGGTTCTATTTAATACCTGCCACCCAGTGATTTATGATGC | |
| | CTTGGAGACAAAGCCCTTATGTCTGTATTTTCACTCATGCCTTCTGAGTGGTGGGGAG | |
| | CCCCTTTTCAGCAGCATTCTGGGTGCCTTTGAAGAGGTACCGGCCTGCTCTCCCCAAA | |
| | AGAATCAGGGCCACAGCTCTTGACAGATCTCCCGGGACAAGATGCGCCTCGGTTTGAG | |
| | CCCTGAGCGTAAGCATTCTGATCCTGAGAGCAGCCAAGGAGATTTTCTGCTGAGCCAA | |
| | ACCCCTTCACATTTTTCTCCTCTTTCCCCAGGTTTTCTTTAAAATCGTTTTTAAATCT | |
| | TAATTTTACTCTCTACTCTTCCTGTATCCACGATACAAGCTCACAGTATATAGCTAGA | |
| | GGAAATGCCATTATGGACCCAACTGTAAGATGGCACATATGTTCGTTTTCCAAGGATC | |
| | AGATGGCATTGCAGGGCCACAGCCAACTGCTGATTGCCAGCACCACCTGAGATGGCAT | |
| | CTCTTGTTTTAAATACATGCACTAACCCTGAAGATTAAGGCCACAGGGGCAGACTGAC | |
| | TAGAGAAGTATAACGTCTGTCTCTGAATGCCATGGTGCCCACCTATGAGACCCTGAGG | |
| | CCGCAGACAAAGAAGAACACCATTCTAGAGGGCTTCCAGCCCTTTCACAAGGTGGACC | |
| | TGTACTGATAGAGAAACACACTCTCTAAGAAGTGCTTACTCACCCTTTTCCAAAGGAG | |
| | CACAGGTGTTGGCCATCAGAAGACACACTGGAGCGCATGGGCCTCTTCACTGTGTGCC | |
| | AAGCTCAGTCACCTCTGATTCAGCCCCTGAGGGTGTCTGCTGCCAGGTGCCCTCAGGG | |
| | TAGGAGAGTGGGAAGTACACGCCAAGCTGGAAAGTGTGTTCTGAAGACCCTCCTCTTG | |
| | CCAAGTGCCTTGCCCATTGCAACCTTGTGTGTGAATTCTAATGGGTTTGAATGGGGGT | |
| | CAGGGTGCATGGGGAAGTTGCTCTGTGGACCTTTGGGACACAGGAATCTTGGACTTAC | |
| | TGGCAGGGGATCCATTCTGAAAGCACCATCCTGTCAACTGTGTTATTGAGGACATTTC | |
| | TTGATGTGAGTATAGTCTGGGTGGCTATTTACTGCCCACTATAGAAATTGTTTGACTA | |
| | TGTAGTGGACCATGTATATATGATAATTATCTATTTTAACACAAAAAAAAAAAAAAAA | |
| | AAAAAAAGGGCCGCCGC | |
| | ORF Start: ATG at 25 | ORF Stop: TAG at 712 |

| | | |
|---|---|---|
| SEQ ID NO: 76 | 229 aa | MW at 26166.1kD |

| | |
|---|---|
| NOV19a, | MRLLALAAAALLARAPAPEVCAALNVTVSPGPVVDYLEGENATLLCHVSQKRRKDSLL |
| CG173357-01 | |
| Protein Sequence | AVRWFFAHSFDSQEALMVKMTKLRWQYYGNFSRSAKRRRLRLLEEQRGALYRLLSVLT |
| | LQPSDQGHYVCRVQEISRHRNKWTAWSNGSSATEMRVISLKASEESSFEKTKETWAFF |
| | EDLYVYAVLVCCMGILSILLFMLVIVWQSVFNKRKSRVRHYLVKCPQNSSGESCH |

Further analysis of the NOV19a protein yielded the following properties shown in Table 19B.

TABLE 19B
Protein Sequence Properties NOV19a

| | |
|---|---|
| PSort | 0.4600 probability located in plasma membrane; |
| analysis: | 0.2000 probability located in lysosome (membrane); |
| | 0.1000 probability located in endoplasmic reticulum |
| | (membrane); 0.1000 probability located in endoplasmic |
| | reticulum (lumen) |
| SignalP | Cleavage site between residues 23 and 24 |
| analysis: | |

A search of the NOV19a protein against the Geneseq database, a proprietary database that contains sequences published in patents and patent publication, yielded several homologous proteins shown in Table 19C.

TABLE 19C
Geneseq Results for NOV19a

| | | NOV19a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU18012 | Human immunoglobulin | 52 . . . 229 | 178/178 (100%) | e−100 |
| | polypeptide SEQ ID No 157 - | 17 . . . 194 | 178/178 (100%) | |
| | Homo sapiens, 194 aa. | | | |
| | [WO20015531S-A2, 02 AUG. | | | |
| | 2001] | | | |
| AAU18070 | Human immunoglobulin | 14 . . . 190 | 174/177 (98%) | 2e−97 |
| | polypeptide SEQ ID No 215 - | 6 . . . 182 | 174/177 (98%) | |
| | Homo sapiens, 203 aa. | | | |
| | [WO200155315-A2, 02 AUG. | | | |
| | 2001] | | | |
| ABB10520 | Human cDNA SEQ ID NO: 828 - | 14 . . . 190 | 174/177 (98%) | 2e−97 |
| | Homo sapiens, 203 aa. | 6 . . . 182 | 174/177 (98%) | |
| | [WO200154474-A2, 02 AUG. | | | |
| | 2001] | | | |
| ABB03217 | Human musculoskeletal system | 14 . . . 190 | 174/177 (98%) | 2e−97 |
| | related polypeptide SEQ ID | 6 . . . 182 | 174/177 (98%) | |
| | NO 1164 - Homo sapiens, 203 | | | |
| | aa. [WO200155367-A1, 02 AUG. | | | |
| | 2001] | | | |
| ABB72358 | Murine protein isolated from | 1 . . . 207 | 170/207 (82%) | 1e−92 |
| | skin cells SEQ ID NO: 682 - | 3 . . . 206 | 185/207 (89%) | |
| | Mus sp, 210 aa. | | | |
| | [WO200190357-A1, 29 NOV. | | | |
| | 2001] | | | |

In a BLAST search of public sequence datbases, the NOV19a protein was found to have homology to the proteins shown in the BLASTP data in Table 19D.

TABLE 19D
Public BLASTP Results for NOV19a

| | | NOV19a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96MX7 | CDNA FLJ31737 fis, clone | 1 . . . 164 | 155/164 (94%) | 1e−83 |
| | NT2RI2007084 - Homo sapiens | 1 . . . 164 | 157/164 (95%) | |
| | (Human), 191 aa. | | | |
| Q93033 | Leukocyte surface protein - | 38 . . . 226 | 47/189 (24%) | 3e−04 |
| | Homo sapiens (Human), 1021 | 426 . . . 602 | 82/189 (42%) | |
| | aa. | | | |
| AAC72013 | IG-LIKE MEMBRANE PROTEIN - | 37 . . . 131 | 27/95 (28%) | 4e−04 |
| | Homo sapiens (Human), 1215 | 712 . . . 806 | 42/95 (43%) | |
| | aa. | | | |
| O75054 | KIAA0466 protein - Homo | 37 . . . 131 | 27/95 (28%) | 4e−04 |
| | sapiens (Human), 1214 aa | 712 . . . 806 | 42/95 (43%) | |
| | (fragment). | | | |
| I39207 | leukocyte surface protein | 38 . . . 226 | 47/189 (24%) | 0.002 |
| | V7 - human, 1021 aa. | 426 . . . 602 | 81/189 (41%) | |

PFam analysis predicts that the NOV19a protein contains the domains shown in the Table 19E.

TABLE 19E
Domain Analysis of NOV19a

| | | Identities/ | |
|---|---|---|---|
| | | Similarities for | |
| Pfam | NOV19a Match | the Matched | Expect |
| Domain | Region | Region | Value |
| ig | 39 . . . 129 | 16/92 (17%) | 2.7e−05 |
| | | 57/92 (62%) | |

### Example 20

The NOV20 clone was analyzed, and the nucleotide and encoded polypeptide sequences are shown in Table 20A.

TABLE 20A
NOV20 Sequence Analysis

| | |
|---|---|
| SEQ ID NO: 77 | 704 bp |

| NOV20a, | ATGGGCGACTGGAGCTTTCTGGGAAGACTCTTAGAAAATGCACAGGAGCACTCCACGG | |
|---|---|---|
| CG50387-01 DNA | | |
| Sequence | TCATCGGCAAGGTTTGGCTGACCGTGCTGTTCATCTTCCGCATCTTGGTGCTGGGGGC | |
| | CGCGGCGGAGGACGTGTGGGGCGATGAGCAGTCAGACTTCACCTGCGACACCCGGCCG | |
| | CCCGCCGTTGCCATCGGGTTCCCACCCTACTATGCGCACACCGCTGCGCCCCTGGGAC | |
| | AGGCCCGCGCCGTGGGCTACCCCGGGGCCCCGCCACCAGCCGCGGACTTCGGCTTGCT | |
| | AGCCCTGACCGAGGCGCGCGGAAAGGGCCAGTCCGCCAAGCTCTACTGCGGCCACCAC | |
| | CACCTGCTGATGACTGAGCAGAACTGGGCCAACCAGGCGGCCGAGCGGCAGCCCCCGG | |
| | CACTCAAGGCTTACCCGGCAGCGTCCACGCCTGCAGCCCCCAGCCCCGTCGGCAGCAG | |
| | CTCCCCGCCACTCGCGCACGAGGCTGAGGCGGGCGCGGCGCCCCTGCTGCTGGATGGG | |
| | AGCGGCAGCAGTCTGGAGGGGAGCGCCCTGGCAGGGACCCCCGAGGAGGAGGAGCAGG | |
| | CCGTGACCACCGCGGCCCAGATGCACCAGCCGCCCTTGCCCCTCGGAGACCCAGGTCG | |
| | GGCCAGCAAGGCCAGCAGGGCCAGCAGCGGGCGGGCCAGACCGGAGGACTTGGCCATC | |
| | TAGTGCCC | |
| | ORF Start: ATG at 1 | ORF Stop: TAG at 697 |

| | | |
|---|---|---|
| SEQ ID NO: 78 | 232 aa | MW at 24185.8kD |

| NOV20a | MGDWSFLGRLLENAQEHSTVIGKVWLTVLFIFRTLVLGAAAEDVWGDEQSDFTCNTRP | |
|---|---|---|
| C050387-01 | | |
| Protein Sequence | PAVAIGFPPYYAHTAAPLGQARAVGYPGAPPPAADFKMLALTEARGKGQSAKLYNGHH | |
| | HLLMTEQNWANQAAERQPPALKAYPAASTPAAPSPVGSSSPPLAHEAEAGAAPLLLDG | |
| | SGSSLEGSALAGTPEEEEQAVTTAAQMHQPPLPLGDPGRASKASRASSGRARPEDLAI | |
| | SEQ ID NO: 79 | 1308 bp |

| NOV20b, | ATGGGCGACTGGAGCTTTCTGGGAAGACTCTTAGAAAATGCACAGGAGCACTCCACGG | |
|---|---|---|
| CG50387-03 DNA | | |
| Sequence | TCATCGGCAAGGTTTGGCTGACCGTGCTGTTCATCTTCCGCATTTTGGTGCTGGGGGC | |
| | CGCGGCCGAGGACGTGTGGGGCGATGAGCAGTCAGACTTCACCTGCGACACCCAGCAG | |
| | CCGGGCTGCGAGAACGTCTGCTACGACAGGGCCTTCCCCATCTCCCACATCCGCTTCT | |
| | GGGCGCTGCAGATCATCTTCGTGTCCACGCCCACCCTCATCTACCTGGGCCACGTGCT | |
| | GCACATCGTGCGCATGGAGGAGAAGAAGAAAGAGAGGGAGGAGGAGGAGCAGCTGTCG | |
| | AGAGAGAGCCCCAGCCCCAAGGAGCCACCGCAGGACAATCCCTCGTCGCGGGACGACC | |
| | GCGGCAGGGTGCGCATGGCCGGCGCGCTGCTGCGGACCTACGTCTTCTACATCATCTT | |
| | CAAGACGCTGTTCGAGGTGGGCTTCATCGCCGGCCAGTACTTTCTGTACGGCTTCGAG | |
| | CTGAAGCCGCTCTACCGCTGCGACCGCTGGCCCTGCCCCAACACGGTGGACTGCTTCA | |
| | TCTCCAGGCCCACGGAGAAGACCATCTTCATCATCTTCATGCTGGCGGTGGCCTGCGC | |
| | GTCACTGCTGCTCGACATGCTGGAGATATACCACCTGGGCTGGAAGCGCTCATGGCAG | |
| | GGCGTGACCAGCCGCCTCGGCCCGGACGCCTCCGAGGCCCCGCTGGGGACAGCCGATC | |
| | CCCCGCCCCTGCCCCCCAGCTCCCGGCCGCCCGCCGTTGCCATCGGGTTCCCCCCCTA | |
| | CTATGCGCACACCGCTGCGCCCCTGGGACAGGCCCGCGCCGTGGGCTACCCCGGGGCC | |
| | CCGCCACCAGCCGCGGACTTCAAAATGCTAGCCCTGACCGAGGCGCGCGGTCAGGGCC | |
| | AGTCCGCCAAGCTCTACAACGGCCACCACCACCTGCTGATGACTGAGCAGGCGTGGGC | |
| | CAACCAGGCGGCCGAGCGGCAGCCCCCGGCGCTCAAGGCTTACCCGGCAOCGTCCACG | |
| | CCTGCAGCCCCCAGCCCCGTCGGCAGCAGCTCCCCGCCACTCGCGCACGAGGCTGAGG | |
| | CGGGCGCGGCGCCCCTGCTGCTGGATGGGAGCGGCAGCAGTCTGGAGGGGAGCGCCCT | |
| | GGCAGGGACCCCCGAGGAGGAGGAGCAGGCCGTGACCACCGCGGCCCAGATGCACCAG | |
| | CCGCCCTTGCCCCTCGGAGACCCAGGTCGGGCCAGCTAGGCCAGCAGGGCCAGCAGCG | |
| | GGCGGGCCAGACCGGAGGACTTGGCCATCTAG | |
| | ORF Start: ATG at 1 | ORF Stop: TAG at 1306 |

| | | |
|---|---|---|
| SEQ ID NO: 80 | 435 aa | MW at 47427.5kD |

| NOV20b, | MGDWSFLGRLLENAQEHSTVIGKVWLTVLFIFRILVLGAAAEDVWGDEQSDFTCNTQQ | |
|---|---|---|
| CG50387-03 | | |
| Protein Sequence | PGCENVCYDRAFPISHIRFWALQIIFVSTPTLIYLGHVLHIVGAEEKKKEREEEEQLK | |
| | RESPSPKEPPQDNPSSRDDRGRVRMAGALLRTYVFNIIFKTLFEVGFIAGQYFLYGFE | |
| | LKPLYRCDRWPCPNTVDCFISRPTEKTIFIIFMLAVACASLLLNMLEIYHLGWKQGKQ | |
| | GVTSRLGPDASEAPLGTADPPPLPPSSRPPAVAIGFPPYYAGTGAPLGQKLIVGYPGA | |
| | PPPADFKMLALTEARGKGQSAKLYNGHHHLLMTEQNWKMQEIMERQPPALAGTYPHST | |
| | PAPSPVGSSSPPLAHEAEAGAAPLLLDGSGSSLEGSTRAGTPEEEEQAVTTKREQMHQ | |
| | PPLPLGDPGRASKASRASSGRARPEDLAI | |
| | SEQ ID NO: 81 | 954 bp |

| NOV20c, | ATGGGCGACTGGAGCTTTCTGGGAAGACTCTTAGCGGATGCACAGGAGCACTCCACGG | |
|---|---|---|
| CG50387-02 DNA | | |
| Sequence | TCATCGGCAAGGTTTGGCTGACCGTGCTGTTCATCTTCCGCATTTTGGTGCTGGGGGC | |
| | CGCGGCCGAGGACGTGTGGGGCGATGAGCAGTCAGACTTCACCTGCGACACCCAGCAG | |
| | CCGGGCTGCGAGAACGTCTGCTACGACAGGGCCTTCCCCATCTCCCACATCCGCTTCT | |
| | GGGCGCTGCAGATCATCTTCGTGTCCACCCCCACCCTCATCTACCTGGGCCACGTGCT | |
| | GCACATCGTGCGCATGGAGGAGAAGAAGAAAGAGAGGGAGGAGGAGGAGCAGCTGAGG | |
| | AGAGAGCCCCAGCCCCAAGGAGCCACCGCAGGACTCCCTCGTCGCGGGACGACCGCGG | |
| | CAGGGTGCGCATGGCCGGCGCGCTGCTGCGGACCTACGTCTTCCATCATCTTCAAGAC | |
| | GCTGTTCGAGGTGGGCTTCATCGCCGGCCAGTACTTTCTGTACGGCTTCGAGCTGAAG | |
| | CCGCTCTACCGCTGCGACCGCTGGCCCTGCCCCCACGGTGGACTGCTTCATCTCCAGG | |
| | CCCACGGAGAAGACCATCTTCATCATCTTCATGCTGGCGGTGGCCTGCGCGTCACTGC | |
| | TGCTCCATGCTGGAGATATACCACCTGGGCTGGGGCTCGCAGGGCGTGACCAGCCGCC | |
| | TCGGCCCGGACGCCTCCGAGGCCCCGCTGGGGACAGCCCATCCCCCGCCCCTGCTGCT | |
| | GGATGGGAGCGGCAGCAGTCTGGAGGGGAGCGCCCTGGCAGGGACCCCCGAGGAGGAG | |
| | GAGCAGGCCGTGACCACCGCGGCCCAGATGCACCAGCCGCCCTTGCCCCTCGGAGACC | |
| | CAGGTCGGGCCAGCAAGGCCAGCAGGGCCAGCAGCGGGCGGGCCAGACCGGAGGACTT | |
| | GGCCATCTAG | |
| | ORF Start: ATG at 1 | ORF Stop: TAG at 952 |

| | | |
|---|---|---|
| SEQ ID NO: 82 | 317 aa | MW at 35397.1kD |

| | |
|---|---|
| NOV20c, | MGDWSFLGRLLENAQEHSTVIGKVWLTVLFIFRILVLGAAAEDVWGDEQSDFTCNTQQ |
| CG50387-02 | |
| Protein Sequence | PGCENVCYDRAFPISHIRFWALQIIFVSTPTLIYLGHVLHIVRMEEKKKEREEEEQLK |
| | RESPSPKEPPQDNPSSRDDRGRVRMAGALLRTYVFNIIFKTLFEVGFIAGQYFLYGFE |
| | LKPLYRCDRWPCPNTVDCFISRPTEKTIFIIFMLAVACASLLLNMLEIYHLGWKKLKQ |
| | GVTSRLGPDASEAPLGTADPPPLLLDGSGSSLEGSALAGTPEEEEQAVTTTAQMHQPP |
| | LPLGDPGRASKASRASSGRARPEDLAI |

Sequence comparison of the above protein sequences yields the following sequence relationships shown in Table 20B.

TABLE 20B
Comparison of NOV20a against NOV20b and NOV20c.

| | | Identities/ |
|---|---|---|
| | | Similarities for |
| Protein | NOV20a Residues/ | the Matched |
| Sequence | Match Residues | Region |
| NOV20b | 55 . . . 232 | 176/178 (98%) |
| | 258 . . . 435 | 178/178 (99%) |
| NOV20C | 147 . . . 232 | 69/86 (80%) |
| | 242 . . . 317 | 74/86 (85%) |

Further analysis of the NOV20a protein yielded the following properties shown in Table 20C.

TABLE 20C
Protein Sequence Properties NOV20a

| | |
|---|---|
| PSort | 0.7900 probability located in plasma membrane; 0.3748 |
| analysis: | probability located in microbody (peroxisome); 0.3000 |
| | probability located in Golgi body; 0.2000 probability located |
| | in endoplasmic reticulum (membrane) |
| SignalP | Cleavage site between residues 42 and 43 |
| analysis: | |

A search of the NOV20a protein against the Geneseq database, a proprietary database that contains sequences published in patents and patent publication, yielded several homologous proteins shown in Table 20D.

TABLE 20D
Geneseq Results for NOV20a

| | | NOV20a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAW49009 | Mouse alpha 3 connexin | 58 . . . 232 | 88/175 (50%) | 2e−38 |
| | protein - Mus sp, 417 aa. | 267 . . . 417 | 109/175 (62%) | |
| | [WO9830677-A1, 16 JUL. 1998] | | | |
| AAW23968 | Connexin protein Cx40 - Homo | 1 . . . 59 | 43/59 (72%) | 4e−20 |
| | sapiens, 358 aa. [WO9802150- | 1 . . . 59 | 48/59 (80%) | |
| | A1, 22 JAN. 1998] | | | |
| AAG00107 | Human secreted protein, SEQ | 1 . . . 59 | 43/59 (72%) | 7e−20 |
| | ID NO: 4188 - Homo sapiens, | 1 . . . 59 | 48/59 (80%) | |
| | 83 aa. [EP1033401-A2, 06 SEP. | | | |
| | 2000] | | | |
| AAB58122 | Lung cancer associated | 1 . . . 59 | 43/59 (72%) | 7e−20 |
| | polypeptide sequence SEQ ID | 48 . . . 106 | 48/59 (80%) | |
| | 460 - Homo sapiens, 124 aa. | | | |
| | [WO200055180-A2, 21 SEP. 2000] | | | |
| ABB05038 | Human NOV3b protein SEQ ID | 1 . . . 59 | 40/59 (67%) | 4e−19 |
| | NO: 12 - Homo sapiens, 543 aa. | 1 . . . 59 | 47/59 (78%) | |
| | [WO200190155-A2, 29 NOV. 2001] | | | |

In a BLAST search of public sequence datbases, the NOV20a protein was found to have homology to the proteins shown in the BLASTP data in Table 20E.

TABLE 20E
Public BLASTP Results for NOV20a

| | | NOV20a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9Y6H8 | Gap junction alpha-3 protein | 55 . . . 232 | 176/178 (98%) | 8e−99 |
| | (Connexin 46) (Cx46) - Homo | 257 . . . 434 | 178/178 (99%) | |
| | sapiens (Human), 434 aa. | | | |
| Q64448 | Gap junction alpha-3 protein | 58 . . . 232 | 88/175 (50%) | 6e−38 |
| | (Connexin 46) (Cx46) - Mus | 266 . . . 416 | 109/175 (62%) | |
| | musculus (Mouse), 416 aa. | | | |
| S25764 | connexin 46 - rat, 416 aa. | 55 . . . 232 | 90/178 (50%) | 2e−35 |
| | | 264 . . . 416 | 107/178 (59%) | |
| P29414 | Gap junction alpha-3 protein | 55 . . . 232 | 90/178 (50%) | 2e−35 |
| | (Connexin 46) (Cx46) - | 263 . . . 415 | 107/178 (59%) | |
| | Rattus norvegicus (Rat), 415 | | | |
| | aa. | | | |
| A45338 | connexin-56 - chicken, 510 | 1 . . . 59 | 56/59 (94%) | 1e−26 |
| | aa. | 1 . . . 59 | 58/59 (97%) | |

PFam analysis predicts that the NOV20a protein contains the domains shown in the Table 20F.

TABLE 20F
Domain Analysis of NOV20a

| | | Identities/ | |
|---|---|---|---|
| | | Similarities for | |
| Pfam | NOV20a | the Matched | Expect |
| Domain | Match Region | Region | Value |
| connexin | 1 . . . 118 | 65/247 (26%) | 1.4e−09 |
| | | 89/247 (36%) | |

### Example 21

The NOV21 clone was analyzed, and the nucleotide and encoded polypeptide sequences are shown in Table 21A.

TABLE 21A
NOV21 Sequence Analysis

| | |
|---|---|
| SEQ ID NO: 83 | 1306 bp |

| | |
|---|---|
| NOV21a, | CTCACTATAGGGCTCGAGCGGGCTTGGGCCCCCCGGGGGCCAAAGGGTTCCCCAAGAA |
| CG52113-01 DNA | |
| Sequence | CCAGAGGAGAAGGCCACCCCGCCTGGAGGCACAGGCCATGAGGGGCTCTCAGGAGGTG |
| | CTGCTGATGTGGCTTCTGGTGTTGGCAGTGGGCGGCACAGAGCACGCCTACCGGCCCG |
| | GCCGTACGGTGTGTGCTGTCCGGGCTCACGGGGACCCTGTCTCCGAGTCGTTCGTCCA |
| | GCGTGTGTACCAGCCCTTCCTCACCACCTGCGACGGGCACCGGGCCTGCAGCACCTAC |
| | CGAACCATCTATAGGACCGCCTACCGCCGCAGCCCTGGGCTGGCCCCTGCCAGGCCTC |
| | GCTACGCGTGCTGCCCCGGCTGGAAGAGGACCAGCGGGCTTCCTGGGGCCTGTGGAGC |
| | AGCAATATGCCAGCCGCCATGCCGGAACGGAGCGAGCTGTGTCCACCCTGGCCGCTGC |
| | CGCTGCCCTGCAGGATGGCGGGGTGACACTTCCCAGTCAGATGTCGATGAATGCAGTG |
| | CTAGGAGGGCCGGCTGTCCCCAGCGCTGCGTCAACACCGCCGGCAGTTACTGGTGCCA |
| | GTGTTGGGAGGGGCACAGCCTGTCTGCAGACGGTACACTCTGTGTGCCCAAGGGAGGG |
| | CCCCCCAGGGTGGCCCCCAACCCGACAGGAGTGGACAGTGCAATGAAGGAAGAAGTGC |
| | AGAGGCTGCAGTCCAGGGTGGACCTGCTGGAGGAGAAGCTGCAGCTGGTGCTGGCCCC |
| | ACTGCACAGCCTGGCCTCGCAGGCACTGGAGCATGGGCTCCCGGACCCCGGCAGCCTC |
| | CTGGTGCACTCCTTCCAGCAGCTCGGCCGCATCGACTCCCTGAGCGAGCAGATTTCCT |
| | TCCTGGAGGAGCAGCTGGGGTCCTGCTCCTGCAAGAAAGACTCGTGACTGCCCAGCGC |
| | CCCAGGCTGGACTTGAGCCCCTCACGCCGCCCTGCAGCCCCCATGCCCCTGCCCAACA |
| | TGCTGGGGGTCCAGAAGCCACCTCGGGGTGACTGAGCGGAAAGCCAGGCAGGGCCTTC |
| | CTCCTCTTCCTCCTCCCCTTCCTCGGGAGGCTCCCCAGACCCTGGCATGGGATGGGCT |
| | GGGATCTTCTCTGTGAATCCACCCCTGGCTACCCCCACCCTGGCTACCCCAACGGCAT |
| | CCCAAGGCCAGGTGGGCCCTCAGCTGAGGGAAGGTACGAGCTCCCTGCTGGAGCCTGG |
| | GACCCATGGCACAGGCCAGGCAGCCCGGAGGCTGGGTGGGGCCTCAGTGGGGGCTGCT |
| | GCCTGACCCCCAGCACAATAAAAATGAAAC |

| | |
|---|---|
| ORF Start: ATG at 96 | ORF Stop: TGA at 915 |

| | | |
|---|---|---|
| SEQ ID NO: 84 | 273 aa | MW at 29617.4 kD |

| | |
|---|---|
| NOV21a, | MRGSQEVLLMWLLVLAVGGTEHAYRPGRRVCAVRAHGDPVSESFVQRVYQPFLTTCDG |
| CG52113-01 | |
| Protein Sequence | HRACSTYRTIYRTAYRRSPGLAPARPRYACCPGWKRTSGLPGACGAAICQPPCRNGGS |
| | CVQPGRCRCPAGWRGDTCQSDVDECSARRGCCPQRCVNTAGSYWCQCWEGHSLSADGT |
| | LCVPKGGPPRVAPNPTGVDSAMKEEVQRLQSRVDLLEEKLQLVLAPLHSLASQALEHG |
| | LPDPGSLLVHSFQQLCRIDSLSEQISFLEEQLGSCSCKKDS |

| | |
|---|---|
| SEQ ID NO: 85 | 1307 bp |

| | |
|---|---|
| NOV21b, | CCAAGCTGGCCCTGCACGGCTGCAAGGGAGGCTCCTGTGGACAGGCCAGGCAGGTGGG |
| CG52113-06 DNA | |
| Sequence | CCTCAGCAGGTGCCTCCAGGCGGCCAGTGGGCCTGAGGCCCCAGCAAGGGCTAGGCTC |
| | CATCTCCAGTCCCAGGACACAGCAGCGGCCACCATGGCCACGCCTGGGCTCCAGCAGC |
| | ATCAGCAGCCCCCAGGACCGGGGAGGCACAGGTGGCCCCCACCACCCGGAGGAGCAGC |
| | TCCTCCCCCTGTCCGGGGGATGACTGATTCTCCTCCGCCAGCCGTAGGGTGTGTGCTG |
| | TCCGGGCTCACGGGGACCCTGTCTCCGAGTCGTTCGTGCAGCGTGTGTACCACCCCTT |
| | CCTCACCACCTGCGACGGGCACCGGGCCTGCAGCACCTACCGCAATATGCCAGCCGCC |
| | ATGCCGGAACGGAGGGAGCTGTGTCCAGCCTGGCCGCTGCCGCTGCCCTGCAGGATGG |
| | CGGGGTGACACTTGCCAGTCAGATGTGGATGAATGCAGTGCTAGGAGGGGCGGCTGTC |
| | CCCAGCGCTGCGTCAACACCGCCGGCAGTTACTGGTGCCAGTGTTGGGAGGGGCACAG |
| | CCTGTCTGCAGACGGTACACTCTGTGTGCCCAAGGGAGGGCCCCCCAGGGTGGCCCCC |
| | AACCCGACAGGAGTGGACAGTGCAATGAAGGAAGAAGTGCAGAGGCTGCAGTCCAGGG |
| | TGGACCTGCTGGAGGAGAAGCTGCAGCTGGTGCTGGCCCCACTGCACAGCCTGGCCTC |
| | GCAGGCACTGGAGCATGGGCTCCCGGACCCCGGCAGCCTCCTGGTGCACTCCTTCCAG |
| | CAGCTCGGCCGCATCGACTCCCTGAGCGAGCAGATTTCCTTCCTGGAGGAGCAGCTGG |
| | GGTCCTGCTCCTGCAAGAAAGACTCGTGACTGCCCAGCGCCCCAGGCTGGACTGAGCC |
| | CCTCACGCCGCCCTGCAGCCCCCATGCCCCTGCCCAACATGCTGCGGGTCCAGAAGCC |
| | ACCTCGGCGTGACTGAGCGGAAGGCCAGGCAGCGCCTTCCTCCTCTTCCTCCTCCCCT |
| | TCCTCGGGAGGCTCCCCAGACCCTGGCATGGGATGGGCTGGGATCTTCTCTGTGAATC |
| | CACCCCTGGCTACCCCCACCCTGGTTACCCCAACGGCATCCCAAGGCCAGGTGGGCCC |
| | TCAGCTGAGGGAAGGTACGAGCTCCCTGCTGGAGCCTGGGACCCATGGCACAGGCCAG |
| | GCAGCCCGGAGGCTGGGTGGGGCCTCAGTGGGGGCTGCTGCCTGACCCCCAGCACAAT |
| | AAAAATGAAACGTGAAAAAAAAAAAAAAAAA |

| | |
|---|---|
| ORF Start: ATG at 150 | ORF Stop: TGA at 897 |

| | | |
|---|---|---|
| SEQ ID NO: 86 | 249 aa | MW at 25902.0 kD |

| | |
|---|---|
| NOV21b, | MATPGLQQHQQPPGPGRhRWPPPPGGAAPAPVRGMTDSPPPAVGCVLSGLTGTLSPSR |
| CG52113-06 | |
| | SCSVCTSPSSPPATGTGPAAPTAICQPPCRNGGSCVQPGRCRCPAGWRGDTCQSDVDE |
| Protein Sequence | |
| | CSARRGGCPQRCVNTAGSYWCQCWEGHSLSADGTLCVPKGGPPRVAPNPTGVDSAMKE |
| | EVQRLQSRVDLLEEKLQLVLAPLHSLASQALEHGLPDPGSLLVHSFQQLCRIDSLSEQ |
| | ISFLEEQLGSCSCKKDS |

| | |
|---|---|
| SEQ ID NO: 87 | 841 bp |

| | |
|---|---|
| NOV21c, | CACCGGATCCACCATGAGGGGCTCTCAGGAGGTGCTGCTGATGTGGCTTCTGGTGTTG |
| 274054261 DNA | |
| Sequence | GCAGTGGGCGGCACAGAGCACGCCTACCGGCCCGGCCGTAGGGTGTGTGCTGTCCGGG |
| | CTCACGGGGACCCTGTCTCCGAGTCGTTCGTGCAGCGTGTGTACCAGCCCTTCCTCAC |
| | CACCTGCGACGGGCACCGGGCCTGCAGCACCTACCGAACCATCTATAGGACCGCCTAC |
| | CGCCGCAGCCCTGGGCTGGCCCCTGCCAGGCCTCGCTACGCGTGCTGCCCCGGCTGGA |
| | AGAGGACCAGCGGGCTTCCTGGGGCCTGTCGAGCAGCAATATGCCAGCCGCCATGCCG |
| | GAACGGAGGGAGCTGTGTCCAGCCTGGCCGCTGCCGCTGCCCTGCAGGATGGCGGCGT |
| | GACACTTGCCAGTCACATGTGGATGAATGCAGTGCTAGGAGGGGCCCCTCTCCCCAGC |
| | GCTGCGTCAACACCGCCGGCAGTTACTGGTGCCAGTGTTGGGAGGGGCACAGCCTGTC |
| | TGCAGACGGTACACTCTGTGTGCCCAAGGGAGGGCCCCCCAGGGTGGCCCCCAACCCG |
| | ACAGGAGTGGACAGTGCAATGAAGGAAGAAGTGCAGAGGCTGCAGTCCACGGTGGACC |
| | TGCTGGAGGAGAAGCTGCAGCTGGTGCTGGCCCCACTGCACAGCCTGGCCTCGCAGGC |
| | ACTGGAGCATGGGCTCCCGGACCCCGGCAGCCTCCTGGTCCACTCCTTCCAGCAGCTC |
| | CGCCGCATCGACTCCCTGAGCGAGCAGATTTCCTTCCTGGAGGAGCAGCTGGGGTCCT |
| | GCTCCTGCAAGAAAGACTCGGTCGACGGC |

| | |
|---|---|
| ORF Start: at 2 | ORF Stop: end of sequence |

| | | |
|---|---|---|
| SEQ ID NO: 88 | 280 aa | MW at 30235.0 kD |

| | |
|---|---|
| NOV21 c, | TGSTMRGSQEVLLMWLLVLAVGGTEHAYRPGRRVCAVRAHGDPVSESFVQRVYQPFLT |
| 274054261 | |
| Protein Sequence | TCDGHRACSTYRTIYRTAYRRSPGLAPARPRYACCPGWKRTSGLPGACGAAICQPPCR |
| | NGGSCVQPGRCRCPAGWRGDTCQSDVDECSARRGGCPQRCVNTAGSYWCQCWEGHSLS |
| | ADGTLCVPKGGPPRVAPNPTGVDSAMKEEVQRLQSRVDLLEEKLQLVLAPLHSLASQA |
| | LEHGLPDPGSLLVHSFQQLGRIDSLSEQISFLEEQLGSCSCKKDSVDG |

| | |
|---|---|
| SEQ ID NO: 89 | 769 bp |

| | |
|---|---|
| NOV21d, | CACCGGATCCTACCGGCCCGGCCGTAGGGTGTGTGCTGTCCGGGCTCACCGGGACCCT |
| 274054299 DNA | |
| Sequence | GTCTCCGAGTCGTTCGTGCAGCGTGTGTACCAGCCCTTCCTCACCACCTGCGACGGGC |
| | ACCGGGCCTGCAGCACCTACCGAACCATCTATAGGACCGCCTACCCCCGCAGCCCTGG |
| | GCTGGCCCCTGCCAGGCCTCGCTACGCGTGCTGCCCCGGCTGGAAGAGGACCAGCGGG |
| | CTTCCTGGGGCCTGTGGAGCAGCAATATGCCAGCCGCCATGCCGGAACGGAGGGAGCT |
| | CTGTCCAGCCTGGCCGCTGCCGCTGCCCTGCAGGATGGCCGGGTGACACTTGCCAGTC |
| | AGATGTGGATGAATGCAGTGCTAGGAGGGGCGGCTGTCCCCAGCGCTGCGTCAACACC |
| | GCCGCCAGTTACTGGTGCCAGTGTTGGGAGGGGCACAGCCTGTCTGCAGACGGTACAC |
| | TCTGTGTGCCCAAGGGAGGGCCCCCCAGGGTGCCCCCCAACCCGACAGGAGTGGACAC |
| | TGCAATGAAGGAAGAAGTGCAGAGGCTGCAGTCCAGGGTGGACCTGCTGGAGGAGAAG |
| | CGCAGCTGGTGCTGGCCCCACTGCACAGCCTGGCCTCGCAGGCACTGGAGCATGCGC |
| | TCCCGGACCCCGGCAGCCTCCTGGTCCACTCCTTCCAGCAGCTCGGCCGCATCGACTC |
| | CTGAGCGAGCAGATTTCCTTCCTGGAGGAGCAGCTGGGGTCCTGCTCCTGCAAGAAA |
| | ACTCGGTCGACCGC |

| | |
|---|---|
| ORF Start: at 2 | ORF Stop: end of sequence |

| | | |
|---|---|---|
| SEQ ID NO: 90 | 256 aa | MW at 27640.9 kD |

| | |
|---|---|
| NOV21d, | TGSYRPGRRVCAVRAHGDPVSESFVQRVYQPFLTTCDGHRACSTYRTIYRTAYRRSPG |
| 274054299 | |
| Protein Sequence | LAPARPRYACCPGWKRTSGLPGACGAAICQPPCRNGGSCVQPGRCRCPAGWRGDTCQS |
| | DVDECSARRGGCPQRCVNTAGSYWCQCWEGHSLSADGTLCVPKGGPPRVAPNPTGVDS |
| | AMKEEVQRLQSRVDLLEEKLQLVLAPLHSLASQALEHGLPDPGSLLVHSFQQLGRIDS |
| | LSEQISFLEEQLGSCSCKKDSVDG |

| | |
|---|---|
| SEQ ID NO: 91 | 841 bp |

| | |
|---|---|
| NOV21e, | CACCGGATCCACCATGAGGGGCTCTCAGGAGGTGCTGCTGATGTGGCTTCTGGTGTTG |
| 274054261 DNA | |
| Sequence | GCAGTGGCCGGCACAGAGCACGCCTACCGGCCCGGCCGTAGCGTGTGTGCTGTCCGGG |
| | CTCACGGGGACCCTGTCTCCGAGTCGTTCGTGCAGCGTGTGTACCAGCCCTTCCTCAC |
| | CACCTGCGACGGGCACCGGGCCTCCACCACCTACCGAACCATCTATAGGACCGCCTAC |
| | CGCCGCAGCCCTGGGCTGGCCCCTGCCAGGCCTCGCTACGCGTGCTGCCCCCGCTGGA |
| | AGAGGACCAGCGGGCTTCCTGGGGCCTGTGGAGCAGCAATATGCCAGCCGCCATGCCG |
| | GAACGGAGGGAGCTGTGTCCAGCCTGGCCGCTGCCGCTGCCCTCCAGGATGGCGGCGT |
| | GACACTTGCCAGTCAGATGTGGATGAATGCAGTGCTAGGAGGGGCGGCTGTCCCCAGC |
| | GCTGCGTCAACACCGCCGGCAGTTACTGGTGCCAGTGTTGGGAGGGGCACAGCCTGTC |
| | TGCAGACGGTACACTCTGTGTGCCCAAGGGAGGGCCCCCCAGGGTGGCCCCCAACCCG |
| | ACAGGAGTGGACAGTGCAATGAAGGAAGAAGTGCAGAGGCTGCAGTCCAGGGTGGACC |
| | TGCTGGAGGAGAAGCTGCAGCTGGTGCTGGCCCCACTGCACAGCCTGGCCTCGCAGGC |
| | ACTGGAGCATGGGCTCCCGGACCCCGGCAGCCTCCTGGTGCACTCCTTCCAGCAGCTC |
| | GGCCGCATCGACTCCCTGAGCGAGCAGATTTCCTTCCTGGAGGAGCAGCTGGGGTCCT |
| | GCTCCTGCAAGAAAGACTCGGTCGACGGC |

| | |
|---|---|
| ORF Start: at 2 | ORF Stop: end of sequence |

| | | |
|---|---|---|
| SEQ ID NO: 92 | 280 aa | MW at 30235.0 kD |

| | |
|---|---|
| NOV21e, | TGSTMRGSQEVLLMWLLVLAVGGTEHAYRPGRRVCAVRAHGDPVSESFVQRVYQPFLT |
| 274054261 | |
| Protein Sequence | TCDGHRACSTYRTIYRTAYRRSPGLAPARPRYACCPGWKRTSGLPGACCAAICQPPCR |
| | NGGSCVQPGRCRCPAGWRGDTCQSDVDECSARRGGCPQRCVNTAGSYWCQCWEGHSLS |
| | ADGTLCVPKGGPPRVAPNPTGVDSAMKEEVQRLQSRVDLLEEKLQLVLAPLHSLASQA |
| | LEHGLPDPGSLLVHSFQQLGRIDSLSEQISFLEEQLGSCSCKKDSVDG |

| | |
|---|---|
| SEQ ID NO: 93 | 769 bp |

| | |
|---|---|
| NOV21f, | CACCGGATCCTACCGGCCCGGCCGTAGGGTGTGTGCTGTCCGGGCTCACGGGGACCCT |
| 274054299 DNA | |
| Sequence | GTCTCCGAGTCGTTCGTGCAGCGTGTGTACCAGCCCTTCCTCACCACCTGCGACGGGC |
| | ACCGGGCCTGCAGCACCTACCGAACCATCTATAGGACCGCCTACCGCCGCAGCCCTGG |
| | GCTGGCCCCTGCCAGGCCTCGCTACGCGTGCTGCCCCGGCTGGAAGAGGACCACCGGG |
| | CTTCCTGGGGCCTGTGGAGCAGCAATATGCCAGCCGCCATGCCGGAACGGAGGGAGCT |
| | GTGTCCAGCCTGGCCGCTGCCGCTGCCCTGCAGGATGGCGGGGTGACACTTGCCAGTC |
| | AGATGTGGATGAATGCAGTCCTAGGAGGGGCGGCTGTCCCCAGCGCTGCGTCAACACC |
| | GCCGGCAGTTACTGGTGCCAGTGTTGGGAGGGGCACAGCCTGTCTGCAGACGGTACAC |
| | TCTGTGTGCCCAAGGGAGGGCCCCCCAGGGTGGCCCCCAACCCGACAGGAGTGGACAG |
| | TGCAATGAAGGAAGAAGTGCAGAGGCTGCAGTCCAGGGTGGACCTGCTGGAGGAGAAG |
| | CTGCAGCTGGTGCTGGCCCCACTGCACAGCCTGGCCTCGCAGGCACTGGAGCATGGGC |
| | TCCCGGACCCCGGCAGCCTCCTGGTGCACTCCTTCCAGCAGCTCGGCCGCATCGACTC |
| | CCTGAGCGAGCAGATTTCCTTCCTGGAGGAGCAGCTGGGGTCCTGCTCCTGCAAGAAA |
| | GACTCGGTCGACGGC |

| | |
|---|---|
| ORF Start: at 2 | ORF Stop: end of sequence |

| | | |
|---|---|---|
| SEQ ID NO: 94 | 256 aa | MW at 27640.9 kD |

| | |
|---|---|
| NOV21f, | TGSYRPGRRVCAVRAHGDPVSESFVQRVYQPFLTTCDGHRACSTYRTIYRTAYRRSPG |
| 274054299 | |
| Protein Sequence | LAPARPRYACCPGWKRTSGLPGACGAAICQPPCRNGGSCVQPGRCRCPAGWRGDTCQS |
| | DVDECSARRGGCPQRCVNTAGSYWCQCWEGHSLSADGTLCVPKGGPPRVAPNPTGVDS |
| | ANKEEVQRLQSRVDLLEEKLQLVLAPLHSLASQALEHGLPDPGSLLVHSFQQLGRIDS |
| | LSEQISFLEEQLGSCSCKKDSVDG |

| | |
|---|---|
| SEQ ID NO: 95 | 1475 bp |

| | |
|---|---|
| NOV21g, | GGGCCTCAGGAGGTGCCTCCAGGCGGCCAGTGGGCCTGAGGCCCCAGCAAGGGCTAGG |
| CG52113-02 DNA | |
| Sequence | GTCCATCTCCAGTCCCAGGACACAGCAGCGGCCACCATGGCCACGCCTGGGCTCCAGC |
| | AGCATCAGCAGCCCCCAGGACCGGGGAGGCACAGGTGGCCCCCACCACCCGGAGGAGC |
| | AGCTCCTGCCCCTGTCCGGGGGATGACTGATTCTCCTCCGCCAGGCCACCCAGAGGAG |
| | AAGGCCACCCCGCCTGGAGGCACAGGCCATGAGGGGCTCTCAGGAGGTGCTGCTGATG |
| | TGGCTTCTGGTGTTGGCAGTGGGCGGCACAGAGCACGCCTACCGGCCCGGCCGTAGGG |
| | TGTGTGCTGTCCGGGCTCACGGGGACCCTGTCTCCGAGTCGTTCGTGCAGCGTGTGTA |
| | CCAGCCCTTCCTCACCACCTGCGACGGGCACCGGGCCTGCAGCACCTACCGAACCATC |
| | TATAGGACCGCCTACCGCCGCAGCCCTGGGCTGGCCCCTGCCAGGCCTCGCTACGCGT |
| | GCTGCCCCGGCTGGAAGAGGACCAGCGGGCTTCCTGCGGCCTGTGGAGCAGCAATATG |
| | CCAGCCGCCATCCCGGAACGGAGGGAGCTGTGTCCAGCCTGGCCGCTGCCGCTGCCCT |
| | GCAGGATGGCGCGGTCACACTTGCCAGTCAGATGTGGATGAATGCAGTGCTAGGAGGG |
| | GCGGCTGTCCCCAGCGCTGCGTCAACACCGCCGGCAGTTACTGGTGCCAGTGTTGGGA |
| | GGGGCACAGCCTGTCTGCAGACGGTACACTCTGTGTGCCCAAGGGAGGGCCCCCCAGG |
| | GTGGCCCCCAACCCGACAGGAGTGGACACTGCAATGAAGGAAGAAGTGCAGAGGCTGC |
| | AGTCCAGGGTGGACCTGCTGGAGGAGAAGCTGCAGCTGGTGCTGGCCCCACTGCACAG |
| | CCTGGCCTCGCAGGCACTGGAGCATGGGCTCCCGGACCCCGGCAGCCTCCTGGTGCAC |
| | TCCTTCCAGCAGCTCGGCCGCATCGACTCCCTGAGCGAGCAGATTTCCTTCCTGGAGG |
| | AGCAGCTGGGGTCCTGCTCCTGCAAGAAAGACTCGTGACTGCCCAGCGCCCCAAGCTG |
| | GACTGAGCCCCTCACGCCGCCCTCCAGCCCCCATGCCCCTGCCCAACATGCTGCGGGT |
| | CCACAACCCACCTCGGGGTGACTGAGCGGAAGGCCAGGCAGGGCCTTCCTCCTCTTCC |
| | TCCTCCCCTTCCTCGGGAGGCTCCCCAGACCCTGGCATCCGATGGGCTGGGATCTTCT |
| | CTGTGAATCCACCCCTGGCTACCCCCACCCTGGCTACCCCAACGGCATCCCAAGGCCA |
| | GGTGGGCCCTCAGCTGAGGGAAGGTACGAGCTCCCTGCTGGAGCCTGGGACCCATGGC |
| | ACAGGCCAGGCAGCCCGGAGGCTGGGTGGCGCCTCAGTGGGGGCTGCTGCCTGACCCC |
| | CAGCACAATAAAAATGAAACGTGAC |

| | |
|---|---|
| ORF Start: at 201 | ORF Stop: TGA at 1080 |

| | | |
|---|---|---|
| SEQ ID NO: 96 | 293 aa | MW at 31986.2 kD |

| | |
|---|---|
| NOV21g, | LILLRQATQRRRPPRLEAQAMRGSQEVLLMWLLVLAVGGTEHAYRPGRRVCAVRAHGD |
| CG52113-02 | |
| Protein Sequence | PVSESFVQRVYQPFLTTCDGHRACSTYRTIYRTAYRRSPGLAPARPRYACCPGWKRTS |
| | GLPGACGAAICQPPCRNGGSCVQPGRCRCPAGWRGDTCQSDVDECSARRGGCPQRCVN |
| | TAGSYWCQCWEGHSLSADGTLCVPKGGPPRVAPNPTGVDSAMKEEVQRLQSRVDLLEE |
| | KLQLVLAPLHSLASQALEHGLPDPGSLLVHSFQQLGRIDSLSEQISFLEEQLGSCSCK |
| | KDS |

| | |
|---|---|
| SEQ ID NO: 97 | 1384 bp |

| | |
|---|---|
| NOV21h, | TTTTTTTTTTTTTTTTTTTTTTTTTTTTTTTTTTTTTTTTTTTTTTTTTTTTTTTTTT |
| CG52113-03 DNA | |
| Sequence | TTTTTTTTTTTTTTTTTTTTTTTTTTTTTTTTTTTTTTTTTTTTTTTTTTTTTTTTTT |
| | TTTTTTGTCACGTTTCATTTTTATTGTGCTGGGGGTCAGGCACCACCCCCCACTGAGG |
| | CCCCACCCAGCCTCCGGGCTGCCTGGCCTGTGCCATGGGTCCCAGGCTCCAGCAGGGA |
| | GCTCGTACCTTCCCTCAGCTGAGGCCCCACCTGGCCTTGGGATGCCGTTGGGGTAGCC |
| | AGGGTGGGGGTAGCCAGGGGTGCATTCACAGAGAAGATCCCAGCCCATCCCATGCCAG |
| | GGTCTGGGGAGCCTCCCGAGGAAGGGGAGGAGGAAGAGGAGGAAGGCCCTGCCTGGCC |
| | TTCCGCTCAGTCACCCCGAGGTGGCTTCTGGACCCCCAGCATGTTGGGCAGGGGCATG |
| | GGGGCTGCAGGGCGGCGTGAGGGGCTCAGTCCAGCCTGGGGCGCTGGGCAGTCACGAG |
| | TCTTTCTTGCAGGAGCAGGACCCCAGCTGCTCCTCCAGGAAGGAAATCTGCTCGCTCA |
| | GGGAGTCGATGCGGCCGAGCTGCTGGAAGGAGTGCACCAGGAGGCTGCCGGGGTCCGG |
| | GAGCCCATGCTCCAGTGCCTGCGAGGCCAGGCTGTGCAGTGGGGCCAGCACCAGCTGC |
| | AGCTTCTCCTCCAGCAGGTCCACCCTGGACTGCAGCCTCTGCACTTCTTCCTTCATTG |
| | CACTGTCCACTCCTGTCGGGTTGGGGGCCACCCTGGCGGGCCCTCCCTTGGGCACACA |
| | GAGTGTACCGTCTGCAGACAGGCTGTGCCCCTCCCAACACTGGCACCAGTAACTGCCG |
| | GCGGTGTTGACGCAGCGCTGGGGACAGCCGCCCCTCCTAGCACTGCATTCATCCACAT |
| | CTGACTGGCAAGTGTCACCCCGCCATCCTGCAGGGCAGCGCCAGCGGCCAGGCTGGAC |
| | ACAGCTCCCTCCGTTCCGGCATGGCGGCTGGCATATTGCTGCTCCACAGGCCCCAGGA |
| | AGCCCGCTGGTCCTCTTCCAGCCGGGGCAGCACGCGTAGCGAGGCCTGGCAGGGGCCA |
| | GCCCAGGGCTGCCGCGGTAGGCGGTCCTATAGATGGTTCGGTAGGTGCTGCAGGCCCG |
| | GTGCCCGTCGCACGTGGTGAGGAAGGGCTGGTACACACGCTGCACGAACGACTCGCAG |
| | ACAGGGTCCCCGTGAGCCCGGACAGCACACACCCTACGGCCGGGCCGGTAGGCGTCCT |
| | CTGTGCCGCCCACTGCCAACACCAGAAGCCACATCAGCAGCACCTCCTGACAGCCCCT |
| | CATGGCCTGTGCCTCCAGGCGGGGTGGCCTTCTCCTCTGGTTCTTGGGCA |

| | |
|---|---|
| ORF Start: ATG at 209 | ORF Stop: TGA at 482 |

| | | |
|---|---|---|
| SEQ ID NO: 98 | 91 aa | MW at 9729.9 kD |

| | |
|---|---|
| NOV21h, | MGPRLQQGARTFPQLRAHLALGCRWGSQGGGSQGWIHREDPSPSHARVWGASRGRGGG |
| CG52113-03 | |
| Protein Sequence | RGGRPCLAFRSVTPRWLLDPQHVGQGHGGCAA |

| | |
|---|---|
| SEQ ID NO: 99 | 1597 bp |

| | |
|---|---|
| NOV21i, | GGGCCTCAGGAGGTGCCTCCAGGCGGCCAGTGGGCCTGAGGCCCCAGCAAGGGCTAGG |
| CG52113-04 DNA | |
| Sequence | GTCCATCTCCAGTCCCAGGACACAGCAGCGGCCACCATGGCCACGCCTGGGCTCCACC |
| | AGCATCAGCAGCCCCCAGGACCGGGGAGGCACAGGTGGCCCCCACCACCCGGAGGAGC |
| | AGCTCCTGCCCCTGTCCGGGGGATCACTGATTCTCCTCCGCCACGCCACCCAGAGGAG |
| | AAGGCCACCCCGCCTGGAGGCACAGGCCATGAGGGGCTCTCAGGAGGTCCTGCTGATG |
| | TGGCTTCTGGTGTTGGCAGTGGGCGGCACAGAGCACGCCTACCGGCCCGGCCGTAGGG |
| | TGTGTGCTGTCCGGGCTCACGGGGACCCTGTCTCCGAGTCGTTCGTGCAGCGTGTGTA |
| | CCAGCCCTTCCTCACCACCTGCGACGGGCACCGCGCCTGCAGCACCTACCGAACCATC |
| | TATAGGACCGCCTACCGCCGCAGCCCTGGGCTGGCCCCTGCCAGGCCTCGCTACGCGT |
| | GCTGCCCCGGCTGGAAGAGGACCAGCGGGCTTCCTGGGGCCTGTGGAGCAGCAATATG |
| | CCAGCCGCCATGCCGGAACGGAGGGAGCTGTGTCCAGCCTGGCCGCTGCCGCTGCCCT |
| | GCAGGATGGCGGGGTGACACTTGCCAGTCAGATGTGGATGAATGCAGTCCTAGGAGGC |
| | GCGGCTGTCCCCAGCGCTGCGTCAACACCGCCGGCAGTTACTGGTCCCAGTGTTGGGA |
| | GGGGCACAGCCTGTCTGCAGACGGTACACTCTGTGTGCCCAAGGGAGGGCCCCCCAGG |
| | GTGGCCCCCAACCCGACAGGAGTGGACAGTGCAATGAAGGAAGAAGTGCAGAGGCTGC |
| | AGTCCAGGGTGGACCTGCTGGAGGAGAAGCTGCAGCTGGTGCTGGCCCCACTGCACAG |
| | CCTGGCCTCGCAGGCACTGGAGCATGGGCTCCCGGACCCCGGCAGCCTCCTGGTGCAC |
| | TCCTTCCAGCAGCTCGGCCGCATCGACTCCCTGAGCGAGCAGATTTCCTTCCTGGAGG |
| | AGCAGCTCGGGTCCTGCTCCTGCAAGAAAGACTCGTGACTGCCCAGCGCCCCAAGCTG |
| | GACTGAGCCCCTCACGCCGCCCTGCAGCCCCCATGCCCCTGCCCAACATGCTGGGGGT |
| | CCAGAAGCCACCTCGGGGTGACTGAGCGGAAGGCCAGGCAGGGCCTTCCTCCTCTTCC |
| | TCCTCCCCTTCCTCGGGAGGCTCCCCAGACCCTGGCATGGGATGGGCTGGGATCTTCT |
| | CTGTGAATCCACCCCTGGCTACCCCCACCCTGGCTACCCCAACGGCATCCCAAGGCCA |
| | GGTGGGCCCTCAGCTGAGGGAAGGTACGAGCTCCCTGCTGGAGCCTGGGACCCATGGC |
| | ACAGGCCAGGCAGCCCGGAGGCTGGGTGGGGCCTCAGTGGGGGCTGCTGCCTGACCCC |
| | CAGCACAATAAAAATGAAACGTGACAAAAAAAAAAAAAAAAAAAAAAAAAAAAAAAAA |
| | AAAAAAAAAAAAAAAAAAAAAAAAAAAAAAAAAAAAAAAAAAAAAAAAAAAAAAAAAA |
| | AAAAAAAAAAAAAAAAAAAAAAAAAAAAAAA |

| | |
|---|---|
| ORF Start: ATG at 261 | ORF Stop: TGA at 1080 |

| | | |
|---|---|---|
| SEQ ID NO: 100 | 273 aa | MW at 29617.4 kD |

| | |
|---|---|
| NOV21i, | MRGSQEVLLMWLLVLAVGGTEHAYRPGRRVCAVRAHGDPVSESFVQRVYQPFLTTCDG |
| CG52113-04 | |
| Protein Sequence | NRACSTYRTIYRTAYRRSPGLAPARPRYACCPGWKRTSGLPGACGAAICQPPCRNGGS |
| | CVQPGRCRCPAGWRGDTCQSDVDECSARRGGCPQRCVNTAGSYWCQCWEGHSLSADGT |
| | LCVPKGGPPRVAPNPTGVDSAMKEEVQRLQSRVDLLEEKLQLVLAPLHSLASQALEHG |
| | LPDPGSLLVHSFQQLGRIDSLSEQISFLEEQLGSCSCKKDS |

| | |
|---|---|
| SEQ ID NO: 101 | 883 bp |

| | |
|---|---|
| NOV21j, | TCACCCCGCCTGGACGCACAGGCCATGAGGGGCTCTCAGGAGGTGCTGCTGATGTGGC |
| CG52113-05 DNA | |
| Sequence | TTCTGGTGTTGGCAGTGGGCGGCACAGAGCACGCCTACCGGCCCGGCCGTAGGGTGTG |
| | TGCTGTCCGGGCTCACGGGGACCCTGTCTCCGAGTCGTTCGTGCAGCGTGTGTACCAG |
| | CCCTTCCTCACCACCTGCGACGGGCACCGGGCCTGCAGCACCTACCGAACCATCTATA |
| | GGACCGCCTACCGCCGCAGCCCTGGGCTGGCCCCTGCCAGGCCTCGCTACGCGTGCTG |
| | CCCCGGCTGGAAGAGGACCAGCGGGCTTCCTGGGGCCTGTGGAGCAGCAATATGCCAG |
| | CCGCCATGCCGGAACGGAGGGAGCTGTGTCCAGCCTGGCCGCTGCCGCTGCCCTGCAG |
| | GATGGCCGGGTGACACTTGCCAGTCAGATGTGGATGAATGCAGTGCTAGGAGGGGCGG |
| | CTGTCCCCAGCGCTGCATCAACACCGCCGGCAGTTACTGGTGCCAGTGTTGGGAGGGG |
| | CACAGCCTGTCTGCAGACGGTACACTCTGTGTGCCCAAGGGAGGGCCCCCCAGGGTGG |
| | CCCCCAACCCGACAGGAGTGGACAGTGCAATGAAGGAAGAAGTGCAGAGGCTGCAGTC |
| | CAGGGTGGACCTGCTGGAGGAGAAGCTGCAGCTGGTGCTGGCCCCACTGCACAGCCTG |
| | GCCTCGCAGGCACTGGAGCATGGGCTCCCGGACCCCGGCAGCCTCCTGGTGCACTCCT |
| | TCCAGCAGCTCGGCCGCATCGACTCCCTGAGCGAGCACATTTCCTTCCTGGAGGAGCA |
| | GCTGGGGTCCTGCTCCTGCAAGAAAGACTCGTGACAGCCCACCGCCCCAGGCTGGACT |
| | GAGCCCCTCACGA |

| | |
|---|---|
| ORF Start: ATG at 25 | ORF Stop: TGA at 844 |

| | | |
|---|---|---|
| SEQ ID NO: 102 | 273 aa | MW at 29631.4 kD |

| | |
|---|---|
| NOV21j, | MRGSQEVLLMWLLVLAVGGTEHAYRPGRRVCAVRAHGDPVSESFVQRVYQPFLTTCDC |
| CG52113-05 | |
| Protein Sequence | HRACSTYRTIYRTAYRRSPGLAPARPRYACCPGWKRTSCLPGACGAAICQPPCRNGGS |
| | CVQPGRCRCPAGWRGDTCQSDVDECSARRGGCPQRCINTAGSYWCQCWEGHSLSADGT |
| | LCVPKGGPPRVAPNPTGVDSAMKEEVQRLQSRVDLLEEKLQLVLAPLHSLASQALEHG |
| | LPDPGSLLVHSFQQLGRIDSLSEQISFLEEQLGSCSCKKDS |

Sequence comparison of the above protein sequences yields the following sequence relationships shown in Table 21B.

TABLE 21B
Comparison of NOV21a against NOV21b through NOV21j.

| | | Identities/ |
|---|---|---|
| | | Similarities for |
| Protein | NOV21a Residues/ | the Matched |
| Sequence | Match Residues | Region |
| NOV21b | 79 . . . 273 | 176/196 (89%) |
| | 54 . . . 249 | 179/196 (90%) |
| NOV21C | 1 . . . 273 | 273/273 (100%) |
| | 5 . . . 277 | 273/273 (100%) |
| NOV21d | 23 . . . 273 | 250/251 (99%) |
| | 3 . . . 253 | 251/251 (99%) |
| NOV21e | 1 . . . 273 | 273/273 (100%) |
| | 5 . . . 277 | 273/273 (100%) |
| NOV21f | 23 . . . 273 | 250/251 (99%) |
| | 3 . . . 253 | 251/251 (99%) |
| NOV21g | 1 . . . 273 | 273/273 (100%) |
| | 21 . . . 293 | 273/273 (100%) |
| NOV21h | No Significant Alignment Found. | |
| NOV21i | 1 . . . 273 | 273/273 (100%) |
| | 1 . . . 273 | 273/273 (100%) |
| NOV21j | 1 . . . 273 | 272/273 (99%) |
| | 1 . . . 273 | 273/273 (99%) |

Further analysis of the NOV21a protein yielded the following properties shown in Table 21C.

TABLE 21C
Protein Sequence Properties NOV21a

| | |
|---|---|
| PSort | 0.5500 probability located in endoplasmic reticulum |
| analysis: | (membrane); 0.1900 probability located in lysosome (lumen); |
| | 0.1000 probability located in endoplasmic reticulum (lumen); |
| | 0.1000 probability located in outside |
| SignalP | Cleavage site between residues 23 and 24 |
| analysis: | |

A search of the NOV21a protein against the Geneseq database, a proprietary database that contains sequences published in patents and patent publication, yielded several homologous proteins shown in Table 21D.

TABLE 21D
Geneseq Results for NOV21a

| | | NOV21a | Identities/ | |
|---|---|---|---|---|
| | | Residues/ | Similarities | |
| Geneseq | Protein/Organism/Length | Match | for the Matched | Expect |
| Identifier | [Patent #, Date] | Residues | Region | Value |
| AAB61609 | Human protein HP03375 - Homo | 1 . . . 273 | 273/273 (100%) | e−168 |
| | sapiens, 273 aa. | 1 . . . 273 | 273/273 (100%) | |
| | [WO200102563-A2, 11 JAN. | | | |
| | 2001] | | | |
| AAM23991 | Human EST encoded protein | 1 . . . 273 | 273/273 (100%) | e−168 |
| | SEQ ID NO: 1516 - Homo | 1 . . . 273 | 273/273 (100%) | |
| | sapiens, 273 aa. | | | |
| | [WO200154477-A2, 02 AUG. | | | |
| | 2001] | | | |
| AAB01376 | Neuron-associated protein - | 1 . . . 273 | 273/273 (100%) | e−168 |
| | Homo sapiens, 273 aa. | 1 . . . 273 | 273/273 (100%) | |
| | [WO200034477-A2, 15 JUN. | | | |
| | 2000] | | | |
| AAB24044 | Human PRO1449 protein | 1 . . . 273 | 273/273 (100%) | e−168 |
| | sequence SEQ ID NO: 8 - Homo | 1 . . . 273 | 273/273 (100%) | |
| | sapiens, 273 aa. | | | |
| | [WO200053754-A1, 14 SEP. | | | |
| | 2000] | | | |
| AAB18675 | Amino acid sequence of a | 1 . . . 273 | 273/273 (100%) | e−168 |
| | human a PRO1449 polypeptide - | 1 . . . 273 | 273/273 (100%) | |
| | Homo sapiens, 273 aa. | | | |
| | [WO200053752-A2, 14 SEP. | | | |
| | 2000] | | | |

In a BLAST search of public sequence datbases, the NOV21a protein was found to have homology to the proteins shown in the BLASTP data in Table 21E.

TABLE 21E
Public BLASTP Results for NOV21a

| | | NOV21a | Identities/ | |
|---|---|---|---|---|
| Protein | | Residues/ | Similarities | |
| Accession | | Match | for the Matched | Expect |
| Number | Protein/Organism/Length | Residues | Portion | Value |
| Q9UHF1 | NOTCH4-like protein | 1 . . . 273 | 273/273 (100%) | e−168 |
| | (Hypothetical 29.6 kDa | 1 . . . 273 | 273/273 (100%) | |
| | protein) - Homo sapiens | | | |
| | (Human), 273 aa. | | | |
| Q96EG0 | Similar to NEU1 protein - | 1 . . . 273 | 272/273 (99%) | e−167 |
| | Homo sapiens (Human), 273 aa. | 1 . . . 273 | 273/273 (99%) | |
| CAC38966 | Sequence 17 from Patent | 1 . . . 273 | 234/273 (85%) | e−136 |
| | WO0119856 - Homo sapiens | 1 . . . 234 | 234/273 (85%) | |
| | (Human), 234 aa. | | | |
| Q9QXT5 | NOTCH4-like protein | 1 . . . 272 | 214/274 (78%) | e−129 |
| | (Vascular endothelial zinc | 4 . . . 277 | 232/274 (84%) | |
| | finger 1) - Mus musculus | | | |
| | (Mouse), 278 aa. | | | |
| Q9DCP5 | Vascular endothelial zinc | 1 . . . 272 | 203/274 (74%) | e−119 |
| | finger 1 - Mus musculus | 4 . . . 264 | 220/274 (80%) | |
| | (Mouse), 265 aa. | | | |

PFam analysis predicts that the NOV21a protein contains the domains shown in the Table 21F.

TABLE 21F
Domain Analysis of NOV21a

| | | Identities/ | |
|---|---|---|---|
| Pfam | NOV21a | Similarities for | Expect |
| Domain | Match Region | the Matched Region | Value |
| EGF | 107 . . . 134 | 15/47 (32%) | 0.0037 |
| | | 22/47 (47%) | |
| EGF | 141 . . . 176 | 15/47 (32%) | 0.0012 |
| | | 25/47 (53%) | |

### Example 22

The NOV22 clone was analyzed, and the nucleotide and encoded polypeptide sequences are shown in Table 22A.

TABLE 22A
NOV22, Sequence Analysis

| | |
|---|---|
| SEQ ID NO: 103 | 1303 bp |

| | |
|---|---|
| NOV22a, | ATATCCAATGGGCTGATTTATCTGACGGTCATGGCCATGGATGCTGGCAACCCCCCTC |
| CG57542-01 DNA | |
| Sequence | TCAACAGCACCGTCCCTGTCACCATCGAGGTGTTTGATGAGAATGACAACCCTCCCAC |
| | CTTCAGCAAGCCCGCCTACTTCGTCTCCGTGGTCGAGAACATCATGGCAGGACCCACG |
| | GTGCTGTTCCTGAATGCCACAGACCTGGACCGCTCCCGGGAGTACGGCCAGGAGTCCA |
| | TCATCTACTCCTTGGAAGGCTCCACCCAGTTTCGGATCAATGCCCGCTCAGGGGAAAT |
| | CACCACCACGTCTCTGCTTGACCCAGAGACCAAGTCTGAATACATCCTCATCGTTCGC |
| | GCAGTGGACGGGGGTGTGGGCCACAACCAGAAAACTGGCATCGCCACCGTAAACATCA |
| | CCCTCCTGGACATCAACGACAACCACCCCACGTGGAAGGACGCACCCTACTACATCAA |
| | CCTGGTGGAGATGACCCCTCCAGACTCTGACGTGACCACGGTGGTGGCTGTTGACCCA |
| | GACCTGGGGGAGAATGGCACCCTGGTGTACAGCATCCAGCCACCCAACAAGTTCTACA |
| | GCCTCAACAGCACCACGGCCAAGATCCGCACCACCCACGCCATGCTGGACCGGGAGAA |
| | CCCCGACCCCCATGAGGCCGAGCTGATGCGCAAAATCGTCGTCTCTGTTACTGACTGT |
| | GGCAGGCCCCCTCTGAAAGCCACCAGCAGTGCCACAGTGTTTGTGAACCTCTTGGATC |
| | TCAATGACAATGACCCCACCTTTCAGAACCTGCCTTTTGTGGCCGAGGTGCTTGAAGG |
| | CATCCCGGCGGGGGTCTCCATCTACCAAGTGGTGGCCATCGACCTCGATGAGGGCCTG |
| | AACGGCCTGGTGTCCTACCGCATCCCGGTGGGCATGCCCCGCATGGACTTCCTCATCA |
| | ACACCAGCAGCGGCGTGGTGGTCACCACCACCGAGCTGGACCGCGAGCGCATCGCGGA |
| | GTACCAGCTGCGGGTGGTGGCCAGTCATGCAGGCACGCCCACCAAGAGCTCCACCAGC |
| | ACGCTCACCATCCATGTGCTGGATGTGAACGACGAGACGCCCACCTTCTTCCCGGCCG |
| | TGTACAATGTGTCTGTGTCCGAGGACGTGCCACGCGAGTTCCGGGTGGTCTGGCTGAA |
| | CTGCACGGACAACGACGTGGGCCTCAATGCAGAGCTCAGCTACTTCATCACAGGTGCT |
| | GCCCCGGCCTCCGCCCACCTGTGCAGGCCTCCTGGGCCCCTGCCTCCACCCCTCCCAG |
| | ATGGACAGCCAGACTAGGTGGGGGCAG |

| | |
|---|---|
| ORF Start: ATG at 31 | ORF Stop: TAG at 1291 |

| | | |
|---|---|---|
| SEQ ID NO: 104 | 420 aa | MW at 45678.7 kD |

| | |
|---|---|
| NOV22a, | MAMDAGNPPLNSTVPVTIEVFDENDNPPTFSKPAYFVSVVENIMAGATVLFLNATDLD |
| CG57542-01 | |
| Protein Sequence | RSREYGQESIIYSLEGSTQFRINARSGEITTTSLLDRETKSEYILIVRAVDGGVGHNQ |
| | KTGIATVNITLLDINDNHPTWKDAPYYINLVEMTPPDSDVTTVVAVDPDLGENGTLVY |
| | SIQPPNKFYSLNSTTGKIRTTHAMLDRENPDPHEAELMRKIVVSVTDCGRPPLKATSS |
| | ATVFVNLLDLNDNDPTFQNLPFVAEVLEGIPAGVSIYQVVAIDLDEGLNGLVSYRMPV |
| | GMPRMDFLINSSSGVVVTTTELDRERIAEYQLRVVASDAGTPTKSSTSTLTIHVLDVN |
| | DETPTFFPAVYNVSVSEDVPREFRVVWLNCTDNDVGLNAELSYFITGAAPASAHLCRP |
| | PGALPPPLPDGQPD |

| | |
|---|---|
| SEQ ID NO: 105 | 1113 bp |

| | |
|---|---|
| NOV22b, | GGATCCGCCACAGACCTGGACCGCTCCCGGGAGTACGGCCAGGAGTCCATCATCTACT |
| 169258612 DNA | |
| Sequence | CCTTGGAAGGCTCCACCCAGTTTCGGATCAATGCCCGCTCAGGGGAAATCACCACCAC |
| | GTCTCTGCTTGACCGAGAGACCAAGTCTGAATACATCCTCATCGTTCGCGCAGTGGAC |
| | GGGGGTGTGGGCCACAACCAGAAAACTGGCATCGCCACCGTAAACATCACCCTCCTGG |
| | ACATCAATGACAACCACCCCACGTGGAACGACGCACCCTACTACATCAACCTGGTGGA |
| | GATGACCCCTCCAGACTCTGATGTGACCACGGTGGTGGCTGTTGACCCAGACCTGGGA |
| | GAGAATGGCACCCTGGTGTACAGCATCCAGCCACCCAACAAGTTCTACACCCTCAACA |
| | GCACCACGGGCAAGATCCGCACCACCCACGCCATGCTGGACCGGGAGAACCCCGACCC |
| | CCATGAGGCCGAGCTGATGCGCAAAATCGTCGTCTCTGTTACTGACTGTGGCAGGCCC |
| | CCTCTGAAAGCCACCAGCAGTGCCACAGTGTTTGTGAACCTCTTGGATCTCAATGACA |
| | ATGACCCCACCTTTCAGAACCTGCCTTTTGTGGCCGAGGTGCTTGAAGGCATCCCGGC |
| | GGGGGTCTCCATCTACCAAGTGGTGGCCATCGACCTCGATGAGGGCCTGAACGGCCTG |
| | GTGTCCTACCGCATGCCGGTGGGCATGCCCCGCATGGACTTCCTCATCAGCAGCAGCA |
| | GCGGCGTGGTGGTCACCACCACCGAGCTGGACCGCGAGCGCATCGCGGAGTACCAGCT |
| | GCGGGTGGTGGCCAGTGATGCAGGCACGCCCACCAAGAGCTCCACCAGCACGCTCACC |
| | ATCCATGTGCTGGATGTGAACGACGAGACGCCCACCTTCTTCCCGGCCGTGTACAATG |
| | TGTCCGTGTCCGAGGACGTGCCACGCGAGTTCCGGGTGGTCTGGCTGAACTGCACGGA |
| | CAACGACGTGGGCCTCAATGCAGAGCTCAGCTATTTCATCACAGGTGCTGCCCCGGCC |
| | TCCGCCCACCTGTGCAGGCCTCCTGGGGCCCTGCCTCCACCCCTCCCAGATGGACAGC |
| | CAGACCTCGAG |

| | |
|---|---|
| ORF Start: at 1 | ORF Stop: end of sequence |

| | | |
|---|---|---|
| SEQ ID NO: 106 | 371 aa | MW at 40369.7 kD |

| | |
|---|---|
| NOV22b, | GSATDLDRSREYGQESIIYSLEGSTQFRINARSGEITTTSLLDRETKSEYILIVRAVD |
| 169258612 | |
| Protein Sequence | GGVGHNQKTGIATVNITLLDINDNHPTWKDAPYYINLVEMTPPDSDVTTVVAVDPDLG |
| | ENGTLVYSIQPPNKFYSLNSTTGKIRTTHAMLDRENPDPHEAELMRKIVVSVTDCGRP |
| | PLKATSSATVFVNLLDLNDNDPTFQNLPFVAEVLEGIPAGVSIYQVVAIDLDEGLNGL |
| | VSYRMPVGMPRMDFLISSSSGVVVTTTELDRERIAEYQLRVVASDAGTPTKSSTSTLT |
| | IHVLDVNDETPTFFPAVYNVSVSEDVPREFRVVWLNCTDNDVGLNAELSYFITGAAPA |
| | SAHLCRPPGALPPPLPDGQPDLE |

| | |
|---|---|
| SEQ ID NO: 107 | 1114 bp |

| | |
|---|---|
| NOV22c, | GGATCCGCCACAGACCTGGACCGCTCCCCGGAGTACGGCCAGGAGTCCATCATCTACT |
| 169258615 DNA | |
| Sequence | CCTTGGAAGGCTCCACCCAGTTTCGGATCAATGCCCGCTCCAGGGGAAATCACCACCA |
| | CGTCTCTGCTTGACCGAGAGACCAAGTCTGAATACATCCTCATCGTTCGCGCAGTGGA |
| | CGGGGGTGTGGGCCACAACCAGAAAACTGGCATCGCCACCGTAAACATCACCCTCCTG |
| | GACATCAATGACAACCACCCCACGTGGAAGGACGCACCCTACTACATCAACCTGGTGG |
| | AGATGACCCCTCCAGACTCTGATGTGACCACGGTGGTGGCTGTTGACCCAGACCTGGG |
| | GGAGAATGGCACCCTGGTGTACAGCATCCAGCCACCCAACAAGTTCTACAGCCTCAAC |
| | AGCACCACGGGCAAGATCCGCACCACCCACGCCATGCTGGACCGGGAGAACCCCGACC |
| | CCCATGAGGCCGAGCTGATGCGCAAAATCGTCGTCTCTGTTACTGACTGTGGCAGGCC |
| | CCCTCTGAAAGCCACCAGCAGTGCCACAGTGTTTGTGAACCTCTTGGATCTCAATGAC |
| | AATGACCCCACCTTTCAGAACCTGCCTTTTGTGGCCGAGGTGCTTGAAGGCATCCCGG |
| | CGGGGGTCTCCATCTACCAAGTGGTGGCCATCGACCTCGATGAGGGCCTGAACGGCCT |
| | GGTGTCCTACCGCATGCTGGTGGGCATGCCCCACATGGACTTCCTCATCAACAGCAGC |
| | AGCGGCGTGGTGGTCACCACCACCGAGCTGGACCGCGAGCGCATCGCGAAGTACCAGC |
| | TGCGGGTGGTGGCCAGTGATGCAGGCACGCCCACCAAGAGCTCCACCAGCACGCTCAC |
| | CATCCATGTGCTGGATGTGAACGACGAGACGCCCACCTTCTTCCCGGCCGTGTACAAT |
| | GTGTCTGTGTCCGAGGACGTGCCACGCGAGTTCCGGGTGGTCTGGCTGAACTGCACGG |
| | ACAACGACGTGGGCCTCAATGCAGAGCTCAGCTACTTCATCACAGGTGCTGCCCCGGC |
| | CTCCGCCCACCTGTGCAGGCCTCCTGGGGCCCTGCCTCCACCCCTCCCAGATGGACAG |
| | CCAGACCTCGAG |

| | |
|---|---|
| ORF Start: at 2 | ORF Stop: end of sequence |

| | | |
|---|---|---|
| SEQ ID NO: 108 | 371 aa | MW at 40080.6 kD |

| | |
|---|---|
| NOV22c, | DPPQTWTAPGSTARSPSSTPWKAPPSFGSMPAPGEITTTSLLDRETKSEYILIVRAVD |
| 169258615 | |
| Protein Sequence | GGVGHNQKTGIATVNITLLDINDNHPTWKDAPYYINLVEMTPPDSDVTTVVAVDPDLG |
| | ENGTLVYSIQPPNKFYSLNSTTGKIRTTHAMLDRENPDPHEAELMRKIVVSVTDCGRP |
| | PLKATSSATVFVNLLDLNDNDPTFQNLPFVAEVLEGTPAGVSIYQVVAIDLDEGLNGL |
| | VSYRMLVGMPHMDFLINSSSGVVVTTTELDRERIAKYQLRVVASDAGTPTKSSTSTLT |
| | IHVLDVNDETPTFFPAVYNVSVSEDVPREFRVVWLNCTDNDVGLNAELSYFITGAAPA |
| | SAHLCRPPGALPPPLPDGQPDLE |

| | |
|---|---|
| SEQ ID NO: 109 | 1114 bp |

| | |
|---|---|
| NOV22d, | GGATCCGCCACAGACCTGGACCGCTCCCCGGGAGTACGGCCAGGAGTCCATCATCTAC |
| 169258621 DNA | |
| Sequence | TCCTTGGAAGGCTCCACCCAGTTTCGGATCAATGCCCGCTCAGGGGAAATCACCACCA |
| | CGTCTCTGCTTGACCGAGAGACCAAGTCTGAATACATCCTCATCGTTCGCGCAGTGGA |
| | CGGGGGTGTGGGCCACAACCAGAAAACTGGCATCGCCACCGTAAACATCACCCTCCTG |
| | GACATCAATGACAACCACCCCACGTGGAAGGACGCACCCTACTACATCAACCTGGTGG |
| | AGATGACCCCTCCAGACTCTGATGTGACCACGGTGGTGGCTGTTGACCCAGACCTGGG |
| | GGAGAATGGCACCCTGGTGTACAGCATCCAGCCACCCAACAAGTTCTACAGCCTCAAC |
| | AGCACCACGGGCAAGATCCGCACCACCCACGCCATGCTGGACCGGGAGAACCCCGACC |
| | CCCATGAGGCCGAGCTGATGCGCAAAATCGTCGTCTCTGTTACTGACTGTGGCAGGCC |
| | CCCTCTGAAAGCCACCAGCAGTGCCACAGTGTTTGTGAACCTCTTGGATCTCAATGAC |
| | AATGACCCCACCTTTCAGAACCTGCCTTTTGTGCCCGAGGTGCTTGAAGGCATCCCGG |
| | CGGGGGTCTCCATCTACCAAGTGGTGGCCATCGACCTCGATGAGGGCCTGAACGGCCT |
| | GGTGTCCTACCGCATGCCGGTGGGCATGCCCCGCATGGACTTCCTCATCAACAGCAGC |
| | AGCGGCGTGGTGGTCACCACCACCGAGCTGGACCGCGAGCGCATCGCCGAGTACCAGC |
| | TGCGGGTGGTGGCCAGTGATGCAGGCACGCCCACCAAGAGCTCCACCAGCACGCTCAC |
| | CATCCATGTGCTGGATGTGAACGACGAGACGCCCACCTTCTTCCCGGCCGTGTACAAT |
| | GTGTCTGTGTCCGAGGACGTGCCACCCGAGTTCCGGGTGGTCTGGCTGAACTGCACGG |
| | ACAACGACGTGGGCCTCAATGCAGAGCTCAGCTACTTCATCACAGGTGCTGCCCCGGC |
| | CTCCGCCCACCTGTGCAGGCCTCCTGGGGCCCTGCCTCCACCCCTCCCAGATGGACAG |
| | CCAGACCTCGAG |

| | |
|---|---|
| ORF Start: at 2 | ORF Stop: end of sequence |

| | | |
|---|---|---|
| SEQ ID NO: 110 | 371 aa | MW at 40487.9 kD |

| | |
|---|---|
| NOV22d, | DPPQTWTAPREYGQESIIYSLEGSTQFRINARSGEITTTSLLDRETKSEYILIVRAVD |
| 169258621 | |
| Protein Sequence | GGVGHNQKTGIATVNITLLDINDNHPTWKDAPYYINLVEMTPPDSDVTTVVAVDPDLG |
| | ENGTLVYSIQPPNKFYSLNSTTGKIRTTHAMLDRENPDPHEAELMRKIVVSVTDCGRP |
| | PLKATSSATVFVNLLDLNDNDPTFQNLPFVAEVLEGIPAGVSIYQVVAIDLDEGLNGL |
| | VSYRMPVGMPRMDFLINSSSGVVVTTTELDRERIAEYQLRVVASDAGTPTKSSTSTLT |
| | IHVLDVNDETPTFFPAVYNVSVSEDVPREFRVVWLNCTDNDVGLNAELSYFITGAAPA |
| | SAHLCRPPGALPPPLPDGQPDLE |

| | |
|---|---|
| SEQ ID NO: 111 | 1114 bp |

| | |
|---|---|
| NOV22e, | GGATCCGCCACAGACCTGGACCGCTCCCGGGAGTACGGCCACGAGTCCATCATCTACT |
| 174307774 DNA | |
| Sequence | CCTTGGAAGGCTCCACCCAGTTTCGGATCAATGCCCGCTCAGGGGAAATCACCACCAC |
| | GTCTCTGCTTGACCGAGAGACCAAGTCTGAATACATCCTCATCGTTCGCGCAGTGGAC |
| | GGGGGTGTGGGCCACAACCAGAAAACTGGCATCGCCACCGTAAACATCACCCTCCTGG |
| | ACATCAACGACAACCACCCCACGTGGAAGGACGCACCCTACTACATCAACCTGGTGGA |
| | GATGACCCCTCCAGACTCTGACGTGACCACGGTGGTGGCTGTTGACCCAGACCTGGGG |
| | GAGAATGGCACCCTGGTGTACAGCATCCAGCCACCCAACAAGTTCTACAGCCTCAACA |
| | GCACCACGGGCAAGATCCGCACCACCCACGCCATGCTGGACCGGGAGAACCCCGACCC |
| | CCATGAGGCCGAGCTGATGCGCAAAATCGTCGTCTCTGTTACTGACTGTGGCAGGCCC |
| | CCTCTGAAAGCCACCAGCAGTGCCACAGTGTTTGTGAACCTCTTGGATCTCAATGACA |
| | ATGACCCCACCTTTCAGAACCTGCCTTTTGTGGCCGAGGTGCTTGAAGGCATCCCGGC |
| | GGGGGTCTCCATCTACCAAGTGGTGGCCATCGACCTCGATGAGGGCCTGAACGGCCTG |
| | GTGTCCTACCGCATGCCGGTGGGCATGCCCCGCATGGACTTCCTCATCAACAGCAGCA |
| | GCGGCGTGGTGGTCACCACCACCGAGCTGGACCGCGAGCGCATCGCGGAGTACCAGCT |
| | GCGGGTGGTGGCCAGTGATGCAGGCACGCCCACCAAGAGCTCCACCAGCACGCTCACC |
| | ATCCATGTGCTGGATGTCAACGACGAGACGCCCACCTTCTTCCCGGCCGTGTACAATG |
| | TGTCTGTGTCCGAGCACGTGCCACGCGAGTTCCCGGTGGTCTGCCTGAACTGCACGCA |
| | CAACGACGTGGGCCTCAATGCAGAGCTCAGCTACTTCATCACAGGGTGCTGCCCCGGC |
| | CTCCGCCCACCTGTGCAGGCCTCCTGGGGCCTTGCCTCCACCCCTCCCAGATGGACAG |
| | CCAGACCTCGAG |

| | |
|---|---|
| ORF Start: at 1 | ORF Stop: end of sequence |

| | | |
|---|---|---|
| SEQ ID NO: 112 | 372 aa | MW at 40670.2 kD |

| | |
|---|---|
| NOV22e, | GSATDLDRSREYGQESIIYSLEGSTQFRINARSGEITTTSLLDRETKSEYILIVRAVD |
| 174307774 | |
| Protein Sequence | GGVGHNQKTGIATVNITLLDINDNHPTWKDAPYYINLVEMTPPDSDVTTVVAVDPDLG |
| | ENGTLVYSIQPPNKFYSLNSTTGKIRTTHAMLDRENPDPHEAELMRKIVVSVTDCGRP |
| | PLKATSSATVFVNLLDLNDNDPTFQNLPFVAEVLEGIPAGVSIYQVVAIDLDEGLNGL |
| | VSYRMPVGMPRMDFLINSSSGVVVTTTELDRERIAEYQLRVVASDAGTPTKSSTSTLT |
| | IHVLDVNDETPTFFPAVYNVSVSEDVPREFRVVWLNCTDNDVGLNAELSYFITGCCPG |
| | LRPPVQASWGLASTPPRWTARPRX |

Sequence comparison of the above protein sequences yields the following sequence relationships shown in Table 22B.

TABLE 22B
Comparison of NOV22a against NOV22b through NOV22e.

| | | Identities/ |
|---|---|---|
| | | Similarities for |
| Protein | NOV22a Residues/ | the Matched |
| Sequence | Match Residues | Region |
| NOV22b | 53 . . . 420 | 366/368 (99%) |
| | 2 . . . 369 | 368/368 (99%) |
| NOV22c | 85 . . . 420 | 333/336 (99%) |
| | 34 . . . 369 | 334/336 (99%) |
| NOV22d | 61 . . . 420 | 360/360 (100%) |
| | 10 . . . 369 | 360/360 (100%) |
| NOV22e | 53 . . . 407 | 346/355 (97%) |
| | 2 . . . 352 | 347/355 (97%) |

Further analysis of the NOV22a protein yielded the following properties shown in Table 22C.

TABLE 22C
Protein Sequence Properties NOV22a

| | |
|---|---|
| PSort | 0.7900 probability located in plasma membrane; 0.3000 |
| analysis: | probability located in microbody (peroxisome); 0.3000 |
| | probability located in Golgi body; 0.2000 probability located |
| | in endoplasmic reticulum (membrane) |
| SignalP | No Known Signal Sequence Predicted |
| analysis: | |

A search of the NOV22a protein against the Geneseq database, a proprietary database that contains sequences published in patents and patent publication, yielded several homologous proteins shown in Table 22D.

TABLE 22D
Geneseq Results for NOV22a

| | | NOV22a | Identities/ | |
|---|---|---|---|---|
| | | Residues/ | Similarities | |
| Geneseq | Protein/Organism/Length | Match | for the Matched | Expect |
| Identifier | [Patent #, Date] | Residues | Region | Value |
| AAM39046 | Human polypeptide SEQ ID NO | 1 . . . 420 | 418/420 (99%) | 0.0 |
| | 2191 - Homo sapiens, 546 aa. | 127 . . . 546 | 419/420 (99%) | |
| | [WO200153312-A1, 26 JUL. | | | |
| | 2001] | | | |
| AAM38969 | Human polypeptide SEQ ID NO | 1 . . . 420 | 418/420 (99%) | 0.0 |
| | 2114 - Homo sapiens, 558 aa. | 139 . . . 558 | 419/420 (99%) | |
| | [WO200153312-A1, 26 JUL. | | | |
| | 2001] | | | |
| AAU01093 | Gene 24 Human secreted | 1 . . . 382 | 382/382 (100%) | 0.0 |
| | protein homologous amino | 68 . . . 449 | 382/382 (100%) | |
| | acid sequence - Homo | | | |
| | sapiens, 449 aa. | | | |
| | [WO200123402-A1, 05 APR. | | | |
| | 2001] | | | |
| ABG03875 | Novel human diagnostic | 85 . . . 395 | 306/402 (76%) | e−161 |
| | protein #3866 - Homo | 994 . . . 1390 | 306/402 (76%) | |
| | sapiens, 1509 aa. | | | |
| | [WO200175067-A2, 11 OCT. | | | |
| | 2001] | | | |
| AAM40755 | Human polypeptide SEQ ID NO | 123 . . . 395 | 262/273 (95%) | e−148 |
| | 5686 - Homo sapiens, 350 aa. | 6 . . . 278 | 263/273 (95%) | |
| | [WO200153312-A1, 26 JUL. | | | |
| | 2001] | | | |

In a BLAST search of public sequence datbases, the NOV22a protein was found to have homology to the proteins shown in the BLASTP data in Table 22E.

TABLE 22E
Public BLASTP Results for NOV22a

| | | NOV22a | Identities/ | |
|---|---|---|---|---|
| Protein | | Residues/ | Similarities | |
| Accession | | Match | for the Matched | Expect |
| Number | Protein/Organism/Length | Residues | Portion | Value |
| AAH32581 | Similar to cadherin related | 1 . . . 420 | 420/420 (100%) | 0.0 |
| | 23 - Homo sapiens (Human), | 642 . . . 1061 | 420/420 (100%) | |
| | 1061 aa. | | | |
| Q96JL3 | KIAA1812 protein - Homo | 1 . . . 395 | 395/395 (100%) | 0.0 |
| | sapiens (Human), 803 aa | 233 . . . 627 | 395/395 (100%) | |
| | (fragment). | | | |
| Q9H251 | Cadherin-23 precursor | 1 . . . 395 | 395/395 (100%) | 0.0 |
| | (Otocadherin) - Homo | 642 . . . 1036 | 395/395 (100%) | |
| | sapiens (Human), 3354 aa. | | | |
| P58365 | Cadherin 23 precursor | 1 . . . 394 | 377/394 (95%) | 0.0 |
| | (Otocadherin) - Rattus | 640 . . . 1033 | 385/394 (97%) | |
| | norvegicus (Rat), 3317 aa. | | | |
| Q99PF4 | Cadherin 23 precursor | 1 . . . 394 | 374/394 (94%) | 0.0 |
| | (Otocadherin) - Mus | 642 . . . 1035 | 384/394 (96%) | |
| | musculus (Mouse), 3354 aa. | | | |

PFam analysis predicts that the NOV22a protein contains the domains shown in the Table 22F.

TABLE 22F
Domain Analysis of NOV22a

| | | Identities/ | |
|---|---|---|---|
| | | Similarities for | |
| Pfam | NOV22a | the Matched | Expect |
| Domain | Match Region | Region | Value |
| cadherin | 35 . . . 128 | 41/108 (38%) | 6.2e−17 |
| | | 67/108 (62%) | |
| cadherin | 142 . . . 238 | 36/112 (32%) | 3.1e−11 |
| | | 67/112 (60%) | |
| cadherin | 254 . . . 345 | 41/107 (38%) | 1.9e−24 |
| | | 69/107 (64%) | |

### Example 23

The NOV23 clone was analyzed, and the nucleotide and encoded polypeptide sequences are shown in Table 23A.

TABLE 23A
NOV23 Sequence Analysis

| | |
|---|---|
| SEQ ID NO: 113 | 1772 bp |

| | |
|---|---|
| NOV2 3a, | CTTTTGCACTGATCATTTCTCTTAATTGGCAGGTAACAAGGAGGGAGCGCATTCTTCC |
| CG57774-01 DNA | |
| Sequence | ACCTTCTGGGTGCTGCTGAGTATCTTTCTGGGAGCAGTGGCCATGCTGTGCAAAGAGC |
| | AAGGGATCACTGTGCTGGGTTTAAATGCGGTATTTGACATCTTGGTGATAGGCAAATT |
| | CAATGTTCTGGAAATTGTCCAGAAGGTACTACATAAGGACAAGTCATTAGAGAATCTC |
| | GGCATGCTCAGGAACGGGGGCCTCCTCTTCAGAATGACCCTGCTCACCTCTGGAGGGG |
| | CTGGGATGCTCTACGTGCGCTGGAGGATCATGGGCACGGGCCCGCCGGCCTTCACCGA |
| | GGTGGACAACCCGGCCTCCTTTGCTGACAGCATGCTGGTGAGGGCCGTAAACTACAAT |
| | TACTACTATTCATTGAATGCCTCGCTGCTGCTGTGTCCCTGGTGGCTGTGTTTTCATT |
| | GGTCAATGGGCTGCATCCCCCTCATTAAGTCCATCAGCGACTGGAGGGTAATTGCACT |
| | TGCAGCACTCTGGTTCTGCCTAATTGGCCTGATATGCCAAGCCCTGTGCTCTGAAGAC |
| | GGCCACAAGAGAAGGATCCTTACTCTGGGCCTGGGATTTCTCGTTATCCCATTTCTCC |
| | CCGCGAGTAACCTGTTCTTCCGAGTGGCCTTCGTGGTCGCACAGCGTGTCCTCTACCT |
| | CCCCAGCGTTGGGTACTGTGTGCTGCTGACTTTTGGATTCGGAGCCCTGAGCAAACAT |
| | ACCAAGAAAAAGAAACTCATTGCCCCTGTCGTGCTGGGAATCTTATTCATCAACACGC |
| | TGAGATGTCTGCTGCGCAGCGGCGAGTGGCGGAGTGAGGAACAGCTTTTCAGAAGTGC |
| | TCTGTCTGTGTGTCCCCTCAATGCTAAGGTACACTACAACATTGGCAAAAACCTCGCT |
| | GATAAACGCAACCAGACAGCTGCCATCAGATACTACCGGGAAGCTGTAAGATTAAATC |
| | CCAAGTATGTTCATGCCATGAATAATCTTGGAAATATCTTAAAAGAAAGGAATGAGCT |
| | ACAGGAAGCTGAGGAGCTGCTGTCTTTGGCTGTTCAAATACAGCCAGACTTTGCCGCT |
| | GCGTGGATGAATCTAGCCATAGTGCAGAATAGCCTGAAACGGTTTGAAGCAGCAGAGC |
| | AAAGTTACCGGACAGCAATTAAACACAGAAGGAAATACCCAGACTGTTACTACAACCT |
| | CGGGCGTCTGTATGCAGATCTCAATCGCCACGTGGATGCCTTGAATCCGTGGAGAAAT |
| | GCCACCGTGCTGAAACCAGAGCACAGCCTGGCCTGGAACAACATGATTATACTCCTCG |
| | ACAATACAGGTAATTTAGCCCAAGCTGAAGCAGTTGGAAGAGACGCACTGGAATTAAT |
| | ACCTAATGATCACTCTCTCATGTTCTCGTTGGCAAACGTGCTGGGGAAATCCCAGAAA |
| | TACAAGGAATCTGAAGCTTTATTCCTCAAGGCAATTAAAGCAAATCCAAATGCTGCAA |
| | GTTACCATGGTAATTTGGCTGTGCTTTATCATCGTTGGGGACATCTAGACTTGGCCAA |
| | GAAACACTATGAAATCTCCTTGCAGCTTGACCCCACGGCATCAGGAACTAAGGAGAAT |
| | TACGGTCTGCTGAGAAGAAAGCTAGAACTAATGCAAAAGAAAGCTGTCTGATCCTGTT |
| | TCCTTCATGTTTTGAGTTTGAGTGTGTGTGTGCATGAGGCATATCATTAATAGTATGT |
| | GGTTACATTTAACCATTTAAAAGTCTTAGACA |

| | |
|---|---|
| ORF Start: ATG at 101 | ORF Stop: TGA at 1673 |

| | | |
|---|---|---|
| SEQ ID NO: 114 | 524 aa | MW at 59138.5 kD |

| | |
|---|---|
| NOV23a, | MLCKEQGITVLGLNAVFDILVIGKFNVLEIVQKVLHKDKSLENLGMLRNGCLLFRMTL |
| CG57774-01 | |
| Protein Sequence | LTSGGAGMLYVRWRIMGTGPPAFTEVDNPASFADSMLVRAVNYNYYYSLNAWLLLCPW |
| | WLCFDWSMGCIPLIKSISDWRVIALAALWFCLIGLICQALCSEDGHKRRILTLGLGFL |
| | VIPFLPASNLFFRVGFVVAERVLYLPSVGYCVLLTFGFGALSKHTKKKKLIAAVVLGI |
| | LFINTLRCVLRSGEWRSEEQLFRSALSVCPLNAKVHYNIGKNLADKGNQTAAIRYYRE |
| | AVRLNPKYVHAMNNLGNILKERNELQEAEELLSLAVQIQPDFAAAWMNLGIVQNSLKR |
| | FEAAEQSYRTAIKHRRKYPDCYYNLGRLYADLNRHVDALNAWRNATVLKPEHSLAWNN |
| | MIILLDNTGNLAQAEAVGREALELIPNDHSLMFSLANVLGKSQKYKESEALFLKAIKA |
| | NPNAASYHGNLAVLYHRWGHLDLAKKHYEISLQLDPTASGTKENYGLLRRKLELMQKK |
| | AV |

| | |
|---|---|
| SEQ ID NO: 115 | 1515 bp |

| | |
|---|---|
| NOV23b, | GAATTCAAATTCAATGTTCTGCAAATTGTCCAGAAGGTACTACATAAGGACAAGTCAT |
| 167200132 DNA | |
| Sequence | TAGAGAATCTCGGCATGCTCAGGAACGGGGGCCTCCTCTTCAGAATGACCCTGCTCAC |
| | CTCTGGAGGGCCTGGGATGCTCTACGTGCGCTGGAGGATCATGGGCACGGGCCCGCCG |
| | GCCTTCACCGAGGTGGACAACCCGGCCTCCTTTGCTGACAGCATGCTGGTGAGGGCCG |
| | TAAACTACAATTACTACTATTCATTGAATGCCTGGCTGCTGCTGTGTCCCTGGTGGCT |
| | GTGTTTTGATTGGTCAATGGGCTGCATCCCCCTCATTAAGTCCATCAGCGACTGGACG |
| | GTAATTGCACTTGCAGCACTCTGGTTCTGCCTAATTGGCCTGATATGCCAAGCCCTGT |
| | GCTCTGAAGACGGCCACAAGAGAAGGATCCTTACTCTGGGCCTGGGATTTCTCGTTAT |
| | CCCATTTCTCCCTGCGAGTAACCTGTTCTTCCGAGTGGGCTTCGTGGTCGCGGAGCGT |
| | GTCCTCTACCTCCCCAGCGTTGGGTACTGTGTGCTGCTGACTTTTGGATTCGGAGCCC |
| | TGAGCAAACATACCAAGAAAAAGAAACTCATTGCCGCTGTCGTGCTGGGAATCTTATT |
| | CATCAACACGCTGAGATGTGTGCTGCGCAGCGGCGAGTGGCGGAGTGAGGAACAGCTT |
| | TTCAGAAGTGCTCTGTCTGTGTGTCCCCTCAATGCTAAGGTTCACTACAACATTGGCA |
| | AAAACCTGGCTGATAAAGGCAACCAGACAGCTGCCATCAGATACTACCGGGAAGCTGT |
| | AAGATTAAATCCCAAGTATGTTCATGCCATGAATAATCTTGGAAATATCTTAAAAGAA |
| | AGGAATGAGCTACAGGAAGCTGAGGAGCTCCTGTCTTTGGCTGTTCAAATACAGCCAG |
| | ACTTTGCCGCTGCGTGGATGAATCTAGGCATAGTGCACAATACCCTGAAACGCTTTGA |
| | AGCAGCAGAGCAAAGTTACCGGACAGCAATTAAACACAGAAGGAAATACCCAGACTGT |
| | TACTACAACCTCGGGCGTCTGTATGCAGATCTCAATCGCCACGTGGATGCCTTGAATG |
| | CGTGGAGAAATGCCACCGTGCTGAAACCAGAGCACAGCCTGGCCTGGAACAACATGAT |
| | TATACTCCTCGACAATACAGGTAATTTAGCCCAAGCTGAAGCAGTTGGAAGAGAGGCA |
| | CTGGAATTAATACCTAATGATCACTCTCTCATGTTCTCGTTGGCAAACGTGCTGGGGA |
| | AATCCCAGAAATACAAGGAATCTGAAGCTTTATTCCTCAAGGCAATTAAAGCAAATCC |
| | AAATGCTGCAAGTTACCATGGTAATTTGGCTGTGCTTTATCATCGTTGGGGACATCTA |
| | GACTTGGCCAAGAAACACTATGAAATCTCCTTGCAGCTTGACCCCACGGCATCAGGAA |
| | CTAAGGAGAATTACGGTCTGCTGAGAAGAAACCTAGAACTAATGCAAAAGAAAGCTGT |
| | CCTCGAG |

| | |
|---|---|
| ORF Start: at 1 | ORF Stop: end of sequence |

| | | |
|---|---|---|
| SEQ ID NO: 116 | 505 aa | MW at 57228.1 kD |

| | |
|---|---|
| NOV23b, | EFKFNVLEIVQKVLHKDKSLENLGMLRNGGLLFRMTLLTSGGAGMLYVRWRIMGTGPP |
| 167200132 | |
| Protein Sequence | AFTEVDNPASFADSMLVRAVNYNYYYSLNAWLLLCPWWLCFDWSMGCIPLIKSISDWR |
| | VIALAALWFCLIGLICQALCSEDGHKRRILTLGLGFLVIPFLPASNLFFRVGFVVAER |
| | VLYLPSVGYCVLLTFGFGALSKHTKKKKLIAAVVLGILFINTLRCVLRSGEWRSEEQL |
| | FRSALSVCPLNAKVHYNIGKNLADKGNQTAAIRYYREAVRLNPKYVHANNNLGNILKE |
| | RNELQEAEELLSLAVQIQPDFAAAWMNLGIVQNSLKRFEAAEQSYRTAIKHRRKYPDC |
| | YYNLGRLYADLNRHVDALNAWRNATVLKPEHSLAWNNMIILLDNTGNLAQAEAVGREA |
| | LELIPNDHSLMFSLANVLGKSQKYKESEALFLKAIKANPNAASYHGNLAVLYHRWGHL |
| | DLAKKHYEISLQLDPTASGTKENYGLLRRKLELMQKKAVLE |

| | |
|---|---|
| SEQ ID NO: 117 | 1515 bp |

| | |
|---|---|
| NOV23c, | GAATTCAAATTCAATGTTCTGGAAATTGTCCAGAAGGTACTACATAAGGACAAGTCAT |
| 167200144 DNA | |
| Sequence | TAGAGAATCTCGGCATGCTCAGGAACGGGGGCCTCCTCTTCAGAATGACCCTGCTCAC |
| | CTCTGGAGGCGCTGGGATGCTCTACGTGCGCTGGAGGATCATGGGCACGGGCCCGCCG |
| | GCCTTCACCGACCTCGACAACCCGGCCTCCTTTGCTGACAGCATGCTGGTGAGGGCCG |
| | TAAACTACAATTACTACTATTCATTCAATGCCTGGCTGCTGCTGTGTCCCTGGTGGCT |
| | GTGTTTTGATTGGTCAATGGGCTGCACCCCCCTCATTAAGTCCATCAGCGACTGGAGG |
| | GTAATTGCACTTGCAGCACTCTGGTTCTGCCTAATTGGCCTGATATGCCAAGCCCTGT |
| | GCTCTGAAGACGGCCACAAGAGAACGATCCTTACTCTGGGCCTGGGATTTCTCGTTAT |
| | CCCATTTCTCCCTGCGAGTAACCTGTTCTTCCGAGTGGGCTTCGTGGTCGCGGAGCGT |
| | GTCCTCTACCTCCCCAGCGTTGGGTACTGTGTGCTGCTGACTTTTGGATTCGGAGCCC |
| | TGAGCAAACATACCAAGAAAAAGAAACTCATTGCCGCTGTCGTGCTGGGAATCTTATT |
| | CATCAACACGCTGAGATGTGTGCTGCGCAGCGGCGAGTGGCGGAGTGAGGAACAGCTT |
| | TTCAGAACTCCTCTGTCTGTGTGTCCCCTCAATGCTAAGGTTCACTACAACATTGGCA |
| | AAAACCTGGCTGATAAAGGCAACCAGACAGCTGCCATCAGATACTACCGGGAAGCTGT |
| | AAGATTAAATCCCAAGTATGTTCATGCCATGAATAATCTTGGAAATATCTTAAAAGAA |
| | AGGAATGAGCTACAGGAAGCTGAGGAGCTGCTGTCTTTGGCTGTTCAAATACAGCCAG |
| | ACTTTGCCGCTGCGTGGATGAATCTAGGCATAGTGCAGAATAGCCTCAAACGGTTTGA |
| | AGCAGCAGAGCAAAGTTACCGGACAGCAATTAAACACAGAAGGAAATACCCAGACTGT |
| | TACTACAACCTCGGGCGTCTGTATGCAGATCTCAATCGCCACGTGGATGCCTTGAATG |
| | CGTGGAGAAATGCCACCGTGCTGAAACCAGAGCACAGCCTGGCCTGGAACAACATGAT |
| | TATACTCCTCGACAATACAGGTAATTTAGCCCAAGCTGAAGCAGTTGGAAGAGAGGCA |
| | CTGGAATTAATACCTAATGATCACTCTCTCATGTTCTCGTTGGCAAACGTGCTGGGGA |
| | AATCCCAGAAATACAAGGAATCTGAAGCTTTATTCCTCAAGGCAATTAAAGCAAATCC |
| | AAATGCTGCAAGTTACCATGGTAATTTGGCTGTGCTTTATCATCGTTGGGGGCATCTA |
| | GACTTGGCCAAGAAACACTATGAAATCTCCTTGCAGCTTGACCCCACGGCATCAGCAA |
| | CTAAGGAGAATTACGGTCTGCTGAGAAGAAAGCTAGAACTAATGCAAAAGAAAGCTGT |
| | CCTCGAG |

| | |
|---|---|
| ORF Start: at 1 | ORF Stop: end at sequence |

| | | |
|---|---|---|
| SEQ ID NO: 118 | 505 aa | MW at 57216.0 kD |

| | |
|---|---|
| NOV23c, | EFKFNVLEIVQKVLHKDKSLENLGMLRNGGLLFRMTLLTSGGAGMLYVRWRIMGTGPP |
| 167200144 | |
| Protein Sequence | AFTEVDNPASFADSMLVRAVNYNYYYSLNAWLLLCPWWLCFDWSMGCTPLIKSISDWR |
| | VIALAALWFCLIGLICQALCSEDGHKRRILTLGLGFLVIPFLPASNLFFRVGFVVAER |
| | VLYLPSVGYCVLLTFGFGALSKHTKKKKLIAAVVLGILFINTLRCVLRSGEWRSEEQL |
| | FRSALSVCPLNAKVHYNIGKNLADKGNQTAAIRYYREAVRLNPKYVHANNNLGNILKE |
| | RNELQEAEELLSLAVQIQPDFAAAWMNLGIVQNSLKRFEAAEQSYRTAIKHRRKYPDC |
| | YYNLGRLYADLNRHVDALNAWRNATVLKPEHSLAWNNMIILLDNTGNLAQAEAVGREA |
| | LELIPNDHSLMFSLANVLGKSQKYKESEALFLKAIKANPNAASYHGNLAVLYHRWGHL |
| | DLAKKHYEISLQLDPTASGTKENYGLLRRKLELMQKKAVLE |

| | |
|---|---|
| SEQ ID NO: 119 | 1515 bp |

| | |
|---|---|
| NOV23d, | GAATTCAAATTCAATGTTCTGGAAATTGTCCAGAAGGTACTACATAAGGACAAGTCAT |
| 169252408 DNA | |
| Sequence | TAGAGAATCTCGGCATGCTCAGGAACGGGGGCCTCCTCTTCAGAATGACCCTGCTCAC |
| | CTCTGGAGGGGCTGGGATGCTCTACGTGCGCTGGAGGATCATGGGCACGGGCCCGCCG |
| | GCCTTCACCGAGGTGGACAACCCGGCCTCCTTTGCTGACAGCATGCTGGTGAGGGCCG |
| | TAAACTACAATTACTACTATTCATTGAATGCCTGGCTGCTGCTGTGTCCCTGGTGGCT |
| | GTGTTTTGATTGGTCAATGGGCTGCATCCCCCTCATTAAGTCCATCAGCGACTGGAGG |
| | GTAATTGCACTTGCAGCACTCTGGTTCTGCCTAATTGGCCTGATATGCCAAGCCCTGT |
| | GCTCTGAAGACGGCCACAAGAGAAGGATCCTTACTCTGGGCCTGAGGATTTCTCGTTAT |
| | CCCATTTCTCCCTGCGAGTAACCTGTTCTTCCGAGTGGGCTTCGTGGTCGCGGAGCGT |
| | GTCCTCTACCTCCCCAGCGTTGGGTACTGTGTGCTGCTGACTTTTGGATTCGGAGCCC |
| | TGAGCAAACATACCAAGAAAAAGAAACTCATTGCCGCTGTCGTGCTGGGAATCTTATT |
| | CATCAACACGCTGAGATGTGTGCTGCGCAGCGGCGAGTGGCGGAGTGAGGAACAGCTT |
| | TTCAGAAGTGCTCTGTCTGTGTGTCCCCTCAATGCTAAGGTTCACTACAACATTGGCA |
| | AAAACCTGGCTGATAAAGGCAACCAGACAGCTGCCATCAGATACTACCGGGAGGCTGT |
| | AAGATTAAATCCCAAGTATGTTCATGCCATGAATAATCTTGGAAATATCTTAAAAGAA |
| | AGGAATGAGCTACAGGAAGCTGAGGAGCTGCTGTCTTTGGCTGTTCAAATACAGCCAG |
| | ACTTTGCCGCTGCGTGGATGAATCTAGGCATAGTGCAGAATAGCCTGAAACGGTTTGA |
| | AGCAGCAGACCAAAGTTACCGGACAGCAATTAAACACAGAAGGAAATACCCAGACTGT |
| | TACTACAACCTCGGGCGTCTGTATGCAGATCTCAATCGCCACGTGGATGCCTTGAATG |
| | CGTGGAGAAATGCCACCGTGCTGAAACCAGAGCACAGCCTGGCCTGGAACAACATGAT |
| | TACACTCCTCGACAATACAGGTAATTTAGCCCAAGCTGAAGCAGTTGGAAGAGAGGCA |
| | CTGGAATTAATACCTAATGATCACTCTCTCATGTTCTCGTTGGCAAACGTGCTGGGGA |
| | AATCCCAGAAATACAAGGAATCTGAAGCTTTATTCCTCAAGGCAATTAAAGCAAATCC |
| | AAATGCTGCAAGTTACCATGGTAATTTGGCTGTGCTTTATCATCGTTGGGGACATCTA |
| | GACTTGGCCAAGAAACACTATGAAATCTCCTTGCAGCTTGACCCCACGGCATCAGGAA |
| | CTAAGGAGAATTACGGTCTGCTGAGAAGAAAGCTAGAACTAATGCAAAAGAAAGCTGT |
| | CCTCGAG |

| | |
|---|---|
| ORF Start: at 1 | ORF Stop: end of sequence |

| | | |
|---|---|---|
| SEQ ID NO: 120 | 505 aa | MW at 57216.0 kD |

| | |
|---|---|
| NOV23d, | EFKFNVLEIVQKVLHKDKSLENLGMLRNGGLLFRMTLLTSGGAGMLYVRWRIMGTCPP |
| 169252408 | |
| Protein Sequence | AFTEVDNPASFADSMLVRAVNYNYYYSLNAWLLLCPWWLCFDWSMGCIPLIKSISDWR |
| | VIALAALWFCLIGLICQALCSEDGHKRRILTLGLGFLVIPFLPASNLFFRVGFVVAER |
| | VLYLPSVGYCVLLTFGFGALSKHTKKKKLIAAVVLGILFINTLRCVLRSGEWRSEEQL |
| | FRSALSVCPLNAKVHYNIGKNLADKGNQTAAIRYYREAVRLNPKYVHAMNNLGNILKE |
| | RNELQEAEELLSLAVQIQPDFAAAWMNLGIVQNSLKRFEAAEQSYRTAIKHRRKYPDC |
| | YYNLGRLYADLNRHVDALNAWRNATVLKPEHSLAWNNMITLLDNTGNLAQAEAVGREA |
| | LELIPNDHSLMFSLANVLGKSQKYKESEALFLKAIKANPNAASYHGNLAVLYHRWGHL |
| | DLAKKHYEISLQLDPTASGTKENYGLLRRKLELMQKKAVLE |

| | |
|---|---|
| SEQ ID NO: 121 | 1515 bp |

| | |
|---|---|
| NOV23e, | GAATTCAAATTCAATGTTCTGGAAATTGTCCAGAAGGTACTACATAAGGACAAGTCAT |
| 169252412 DNA | |
| Sequence | TAGAGAATCTCGGCATGCTCAGGAACGGGGGCCTCCTCTTCAGAATGACCCTGCTCAC |
| | CTCTGGAGGGGCTGGGATGCTCTACGTGCGCTGCACGATCATGGGCACGGGCCCGCCG |
| | GCCTTCACCGAGGTGGACAACCCGGCCCCCTTTGCTGACACCATGCTGGTGAGGGCCG |
| | TAAACTACAATTACTACTATTCATTGAATGCCTGGCTGCTGCTGTGTCCCTGGTGGCT |
| | GTGTTTTGATTGGTCAATGGGCTGCATCCCCCTCATTAAGTCCATCAGCGACTGGAGG |
| | GTAATTGCACTTGCAGCACTCTGGTTCTGCCTAATTGGCCCGATATGCCAAGCCCTGT |
| | GCTCTGAAGACGGCCACAAGAGAAGGATCCTTACTCTGGGCCTGGGATTTCTCGTTAT |
| | CCCATTTCTCCCTGCGAGTAACCTGTTCTTCCCAGTGGGCTTCGTGGTCGCGGAGCGT |
| | GTCCTCTACCTCCCCAGCGTTGGGTACTGTGTUCTGCTCACTTTTGGATTCGGAGCCC |
| | TGAGCAAACATACCAAGAAAAAGAAACTCATTGCCGCTGTCGTGCTGCCAATCTTATT |
| | CATCAACACGCTGAGATGTGTGCTGCGCAGCGGCGAGTGGCGGACTGAGCAACAGCTT |
| | TTCAGAAGTGCTCTGTCTGTGTGTCCCCTCAATGCTAAGGTTCACTACAACATTGCCA |
| | AAAACCTGGCTGATAAAGGCAACCAGACAGCTGCCATCAGATACTACCGGCAAGCTGT |
| | AAGATTAAATCCCAAGTATGTTCATGCCATGAATAATCTTGGAAATATCTTAAAAGAA |
| | AGGAATGAGCTACAGGAAGCTGAGGAGCTGCTGTCTTTGGCTGTTCAAATACAGCCAC |
| | ACTTTGCCGCTGCCTGGATGAATCTAGGCATAGTGCAGAATAGCCTGAAACGGTTTGA |
| | AGCAGCAGAGCAAAGTTACCGGACAGCAATTAAACACAGAAGGAAATACCCAGACTGT |
| | TACTACAACCTCGGGCGTCTGTATGCAGATCTCAATCCCCACGTGGATGCCTTGAATG |
| | CGTGGAGAAATGCCACCGTGCTGAAACCAGAGCACAGCCTGGCCTGGAACAACATGAT |
| | TATACTCCTCGACAATACAGGTAATTTAGCCCAAGCTGAAGCAGTTGGAACAGAGGCA |
| | CTGGAATTAATACCTAATGATCACTCTCTCATGTTCTCGTTGGCAAACGTGCTGCGCA |
| | AATCCCAGAAATACAAGGAATCTGAAGCTTTATTCCTCAAGGCAATTAAAGCAAATCC |
| | AAATGCTGCAAGTTACCATGGTAATTTGGCTGTGCTTTATCATCGTTGGGGACATCTA |
| | GACTTGGCCAAGAAACACTATGAAATCTCCTTGCAGCTTGACCCCACGGCATCAGGAA |
| | CTAAGGAGAATTACGGTCTGCTGAGAAGAAAGCTAGAACTAATGCAAAAGAAAGCTGT |
| | CCTCGAG |

| | |
|---|---|
| ORF Start: at 1 | ORF Stop: end of sequence |

| | | |
|---|---|---|
| SEQ ID NO: 122 | 505 aa | MW at 57222.1 kD |

| | |
|---|---|
| NOV23e, | EFKFNVLEIVQKVLHKDKSLENLGMLRNGGLLFRMTLLTSGGAGMLYVRWRIMGTGPP |
| 169252412 | |
| Protein Sequence | AFTEVDNPAPFADSMLVRAVNYNYYYSLNAWLLLCPWWLCFDWSMGCIPLIKSISDWR |
| | VIALAALWFCLIGPICQALCSEDGHKRRILTLGLGFLVIPFLPASNLFFRVGFVVAER |
| | VLYLPSVGYCVLLTFGFGALSKHTKKKKLIAAVVLGILFINTLRCVLRSGEWRSEEQL |
| | FRSALSVCPLNAKVHYNIGKNLADKGNQTAAIRYYREAVRLNPKYVHANNNLGNILKE |
| | RNELQEAEELLSLAVQIQPDFAAAWMNLGIVQNSLKRFEAAEQSYRTAIKHRRKYPDC |
| | YYNLGRLYADLNRHVDALNAWRNATVLKPEHSLAWNNMIILLDNTGNLAQAEAVGREA |
| | LELIPNDHSLMFSLANVLGKSQKYKESEALFLKAIKANPNAASYHGNLAVLYHRWGHL |
| | DLAKKHYEISLQLDPTASGTKENYGLLRRKLELMQKKAVLE |

| | |
|---|---|
| SEQ ID NO: 123 | 1515 bp |

| | |
|---|---|
| NOV23f, | GAATTCAAATTCAATGTTCTGGAAATTGTCCAGAAGGTACTACATAAGGACAAGTCAT |
| 169252424 DNA | |
| Sequence | TAGAGAATCTCGGCATGCTCAGGAACGGGGGCCTCCTCTTCAGAATGACCCTGCTCAC |
| | CTCTGGAGGGGCTGGGATGCTCTACGTGCGCTGGAGGATCATGGGCACGGGCCCGCCG |
| | GCCTTCACCGAGGTGGACAACCCGGCCTCCTTTGCTGACAGCATGCTGGTGAGGGCCG |
| | TAAACTACAATTACTACTATTCATTGAATGCCTGGCTGCTGCTGTGTCCCTGGTGGCT |
| | GTGTTTTGATTGGTCAATGGGCTGCATCCCCCTCATTAAGTCCATCAGCGACTGGAGG |
| | GTAATTGCACTTGCAGCACTCTGGTTCTGCCTAATTGGCCTGATATGCCAAGCCCTGT |
| | GCTCTGAAGACGGCCACAAGAGAAGGATCCTTACTCTGGGCCTGGGATTTCTCGTTAT |
| | CCCATTTCTCCCTCCCAGTAACCTGTTCTTCCGAGTGGGCTTCGTGGTCGCGGAGCGT |
| | GTCCTCTACCTCCCCAGCGTTGGGTACTGTGTGCTGCTGACTTTTGGATTCGGAGCCC |
| | TGAGCAAACATACCAAGAAAAAGAAACTCATTGCCGCTGTCGTGCTGGGAATCTTATT |
| | CATCAACACGCTGAGATGTGTGCTGCGCAGCGGCGAGTGGCGGAGTGAGGAACAGCTT |
| | TTCAGAAGTGCTCTGTCTGTGTGTCCCCTCAATGCTAAGGTTCACTACAACATTGGCA |
| | AAAACCTGGCTGATAAACGCAACCAGACAGCTGCCATCAGATACTACCCCGAAGCTGT |
| | AAGATTAAATCCCAAGTATGTTCATGCCATGAATAATCTTGGAAATATCTTAAAAGAA |
| | AGGAATGAGCTACAGGAAGCTGAGGAGCTGCTGTCTTTGGCTGTTCAAATACAGCCAG |
| | ACTTTGCCGCTGCGTGGATGAATCTAGGCATAGTGCAGAATAGCCTGAAACGGTTTGA |
| | AGCAGCAGAGCAAAGTTACCGGACAGCAATTAAACACAGAAGGAAATACCCAGACTGT |
| | TACTACAACCTCGGGCGTCTGTATGCAGATCTCAATCGCCACGTGGATGCCTTCAATG |
| | CGTGGAGAAATGCCACCGTGCTGAAACCAGAGCACAGCCTGGCCTGGAACAACATGAT |
| | TATACTCCTCGACAATACAGGTAATTTAGCCCAAGCTGAAGCAGTTGGAAGAGAGGCA |
| | CTGGAATTAATACCTAATGATCACTCTCTCATGTTCTCGTTGGCAAACGTGCTGGGGA |
| | AATCCCAGAAATACAAGGAATCTGAAGCTTTATTCCTCAAGGCAATTAAAGCAAATCC |
| | AAATGCTGCAAGTTACCATGGTAATTTGGCTGTGCTTTATCATCGTTGGGGACATCTA |
| | GACTTGGCCAAGAAACACTATGAAATCTCCTTGCAGCTTGACCCCACGGCATCAGGAA |
| | CTAAGGAGAATTACGGTCTGCTGAGAGGAAAGCTAGAACTAATGCAAAAGAAAGCTGT |
| | CCTCGAG |

| | |
|---|---|
| ORF Start: at 1 | ORF Stop: end of sequence |

| | | |
|---|---|---|
| SEQ ID NO: 124 | 505 aa | MW at 57128.9 kD |

| | |
|---|---|
| NOV23t, | EFKFNVLEIVQKVLHKDKSLENLGMLRNGGLLFRMTLLTSGGAOMLYVRWRIMGTGPP |
| 169252424 | |
| Protein Sequence | AFTEVDNPASFADSMLVRAVNYNYYYSLNAWLLLCPWWLCFDWSMGCIPLIKSISDWR |
| | VIALAALWFCLICLICQALCSEDGHKRRILTLGLGFLVIPFLPASNLFFRVGFVVAER |
| | VLYLPSVGYCVLLTFGFGALSKHTKKKKLIAAVVLGILFINTLRCVLRSGEWRSEEQL |
| | FRSALSVCPLNAKVHYNIGKNLADKGNQTAAIRYYREAVRLNPKYVHAMNNLGNILKE |
| | RNELQEAEELLSLAVQIQPDFAAAWMNLGIVQNSLKRFEAAEQSYRTAIKHRRKYPDC |
| | YYNLGRLYADLNRHVDALNAWRNATVLKPEHSLAWNNMIILLDNTGNLAQAEAVGREA |
| | LELIPNDHSLMFSLANVLGKSQKYKESEALFLKAIKANPNAASYHGNLAVLYHRWGHL |
| | DLAKKHYEISLQLDPTASGTKENYGLLRGKLELMQKKAVLE |

| | |
|---|---|
| SEQ ID NO: 125 | 1515 bp |

| | |
|---|---|
| NOV23 g, | GAATTCAAATTCAATGTTCTGGAAATTGTCCAGAAGGTACTACATAAGGACAAGTCAT |
| 169252469 DNA | |
| Sequence | TAGAGAATCTCGGCATGCTCAGGAACGGGGGCCTCCTCTTCAGAATGACCCTGCTCAC |
| | CTCTGGAGGGGCTGGGATGCTCTACGTGCGCTGGAGGATCATGGGCACGGGCCCGCCG |
| | GCCTTCACCGAGGTGGACAACCCGGCCTCCTTTGCTGACAGCATGCTGGTGAGGGCCG |
| | TAAACTACAATTACTACTATTCATTGAATGCCTGGCTGCTGCTGTGTCCCTGGTGGCT |
| | GTGTTTTGATTGGTCAATGGGCTGCATCCCCCTCATTAAGTCCATCAGCGACTGGAGG |
| | GTAATTGCACTTGCAGCACTCTGGTTCTGCCTAATTGGCCTGATATGCCAAGCCCTGT |
| | GCTCTGAAGACGGCCACAAGAGAAGGATCCTTACTCTGGGCCTGGGATTTCTCGTTAT |
| | CCCATTTCTCCCCGCGAGTAACCTGTTCTTCCGAGTGGGCTTCGTGGTCGCGGAGCGT |
| | GTCCTCTACCTCCCCAGCGTTGGGTACTGTGTGCTGCTGACTTTTGGATTCGGAGCCC |
| | TGAGCAAACATACCAAGAAAAAGAAACTCATTGCCGCTGTCGTGCTGGGAATCTTATT |
| | CATCAACACGCTGAGATGTGTGCTGCGCAGCGGCGAGTGGCGGAGTGAGGAACAGCTT |
| | TTCAGAAGTGCTCTGTCTGTGTGTCCCCTCAATGCTAAGGTTCACTACAACATTGGCA |
| | AAAACCTGGCTGATAAAGGCAACCAGACAGCTGCCATCACATACTACCGGGAAGCTGT |
| | AAGATTAAATCCCAAGTATGTTCATGCCATGAATAATCTTGGAAATATCTTAAAAGAA |
| | AGGAATGAGCTACAGGAAGCTGAGGAGCTGCTGTCTTTGGCTGTTCAAATACAGCCAG |
| | ACTTTGCCGCTGCGTCCATGAATCTAGGCATAGTGCAGAATAGCCTGAAACGGTTTGA |
| | AGCAGCAGAGCAAAGTTACCGGACAGCAATTAAACACAGAAGGAAATACCCAGACTGT |
| | TACTACAACCTCGGGCGTCTGTATGCAGATCTCAATCGCCACGTGGATGCCTTGAATG |
| | CGTGGAGAAATGCCACCGTGCTGAAACCAGAGCACAGCCTGGCCTGGAACAACATGAT |
| | TATACTCCTCGACAATACAGGTAATTTAGCCCAAGCTGAAGCAGTTGGAAGAGAGGCA |
| | CTGGAATTAATACCTAATGATCACTCTCTCATGTTCTCGTTGGCAAACGTGCTGGGGA |
| | AATCCCAGAAATACAAGCAATCTGAAGCTTTATTCCTCAAGGCAATTAAAGCAAATCC |
| | AAATCCTGCAAGTTACCATGGTAATTTGGCTGTGCTTTATCATCGTTGGGGACATCTA |
| | GACTTGGCCAAGAAACACTATGAAATCTCCTTGCAGCTTGACCCCACGGCATCAGGAA |
| | CTAAGGAGAATTACGGTCTGCTGAGAAGAAAGCTAGAACTAATGCAAAAGAAAGCTGT |
| | CCTCGAG |

| | |
|---|---|
| ORF Start: at 1 | ORF Stop: end of sequence |

| | | |
|---|---|---|
| SEQ ID NO: 126 | 1505 aa | W at 57228.1 kD |

| | |
|---|---|
| NOV23g, | EFKFNVLEIVQKVLHKDKSLENLGMLRNGGLLFRMTLLTSGGAGMLYVRWRIMGTGPP |
| 169252469 | |
| Protein Sequence | AFTEVDNPASFADSMLVRAVNYNYYYSLNAWLLLCPWWLCFDWSMGCIPLIKSISDWR |
| | VIALAALWFCLICLICQALCSEDGHKRRILTLGLGFLVIPFLPASNLFFRVGFVVAER |
| | VLYLPSVGYCVLLTFGFGALSKHTKKKKLIAAVVLGILFINTLRCVLRSGEWRSEEQL |
| | FRSALSVCPLNAKVHYNIGKNLADKGNQTAAIRYYREAVRLNPKYVHAMNNLGNILKE |
| | RNELQEAEELLSLAVQIQPDFAAAWMNLGIVQNSLKRFEAAEQSYRTAIKHRRKYPDC |
| | YYNLGRLYADLNRHVDALNAWRNATVLKPEHSLAWNNMIILLDNTGNLAQAEAVGREA |
| | LELIPNDHSLMFSLANVLGKSQKYKESEALFLKAIKANPNAASYNGNLAVLYHRWGHL |
| | DLAKKHYEISLQLDPTASGTKENYGLLRRKLELMQKKAVLE |

| | |
|---|---|
| SEQ ID NO: 127 | 1515 bp |

| | |
|---|---|
| NOV23h, | GAATTCAAATTCAATGTTCTGGAAATTGTCCAGAAGGTACTACATAAGGACAAGTCAT |
| 169252475 DNA | |
| Sequence | TAGAGAATCTCGGCATGCTCAGGAACGGGGGCCTCCTCTTCAGAATGACCCTGCTCAC |
| | CTCTGGAGGGGCTGGGATGCTCTACGTGCGCTGGAGGATCATGGGCACGGGCCCGCCG |
| | GCCTTCACCGAGGTGGACAACCCGGCCTCCTTTGCTGACAGCATGCTGGTGAGGGCCG |
| | TAAACTACAATTACTACTATTCATTGAATGCCTGGCTGCTGCTGTGTCCCTGGTGGCT |
| | GTGTTTTGATTGGTCAATGGGCTGCATCCCCCTCATTAAGTCCATCAGCGACTGGAGG |
| | GTAATTGCACTTGCAGCACTCTGGTTCTGCCTAATTGGCCTGATATGCCAAGCCCTGT |
| | GCTCTGAAGACGGCCACAAGAGAAGGATCCTTACTCTGGGCCTGCGATTTCTCGTTAT |
| | CCCATTTCTCCCCGCGAGTAACCTGTTCTTCCGAGTGGGCTTCGTGGTCGCGGAGCGT |
| | GTCCTCTACCTCCCCAGCGTTGGGTACTGTGTGCTGCTGACTTTTGGATTCGGAGCCC |
| | TGAGCAAACATACCAAGAAAAAGAAACTCATTGCCGCTGTCGTGCTGGGAATCTTATT |
| | CATCAACACGCTGAGATGTGTGCTGCGCAGCGGCGAGTGGCGGAGTGAGGAACAGCTT |
| | TTCAGAAGTGCTCTGTCTGTGTGTCCCCTCAATGCTAAGGTTCACTACAACATTGGCA |
| | AAAACCTGGCTGATAAAGGCAACCAGACAGCAGCCATCAGATACTACCGGGAAGCTGT |
| | AAGATTAAATCCCAAGTATGTTCATGCCATGAATAATCTTGGAAATATCTTAAAAGAA |
| | AGGAATGAGCTACAGGAAGCTGAGGAGCTGCTGTCTTTGGCTGTTCAAATACAGCCAG |
| | ACTTTGCCGCTGCGTGGATGAATCTAGGCATAGTGCAGAATAGCCTGAAACGGTTTGA |
| | AGCAGCAGAGCAAAGTTACCGGACAGCAATTAAACACAGAAGGAAATACCCAGACTGT |
| | TACTACAACCTCGGGCGTCTGTATGCAGATCTCAATCGCCACGTGGATGCCTTGAATG |
| | CGTGCACAAATGCCACCGTGCTGAAACCAGACCACAGCCTGGCCTCGAACAACATGAT |
| | TATACTCCTCGACAATACAGGTAATTTAGCCCAAGCTGAAGCAGTTGGAAGAGAGGCA |
| | CTGGAATTAATACCTAATGATCACTCTCTCATGTTCTCGTTGGCAAACGTGCTGGGGA |
| | AATCCCAGAAATACAAGGAATCTGAAGCTTTATTCCTCAAGGCAATTAAAGCAAATCC |
| | AAATGCTGCAAGTTACCATGGTAATTTGGCTGTGCTTTATCATCGTTGGGGACATCTA |
| | GGCTTGGCCAAGAAACACTATGAAATCTCCTTGCAGCTTGACCCCACGGCATCAGGAA |
| | CTAAGGAGAATTACGGTCTGCTGAGAAGAAAGCTAGAACTAATGCAAAAGAAAGCTGT |
| | CCTCGAG |

| | |
|---|---|
| ORF Start: at 1 | ORF Stop: end of sequence |

| | | |
|---|---|---|
| SEQ ID NO: 128 | 505 aa | MW at 57170.1 kD |

| | |
|---|---|
| NOV23h, | EFKFNVLEIVQKVLHKDKSLENLGMLRNGGLLFRMTLLTSGGAGMLYVRWRIMGTGPP |
| 169252475 | |
| Protein Sequence | AFTEVDNPASFADSMLVRAVNYNYYYSLNAWLLLCPWWLCFDWSMGCIPLIKSISDWR |
| | VIALAALWFCLIGLICQALCSEDGHKRRILTLGLGFLVIPFLPASNLFFRVGFVVAER |
| | VLYLPSVGYCVLLTFGFGALSKHTKKKKLIAAVVLGILFINTLRCVLRSGEWRSEEQL |
| | FRSALSVCPLNAKVHYNIGKNLADKGNQTAAIRYYREAVRLNPKYVHAMNNLGNILKE |
| | RNELQEAEELLSLAVQIQPDFAAAWMNLGIVQNSLKRFEAAEQSYRTAIKHRRKYPDC |
| | YYNLGRLYADLNRHVDALNAWRNATVLKPEHSLAWNNMIILLDNTGNLAQAEAVGREA |
| | LELIPNDHSLMFSLANVLGKSQKYKESEALFLKAIKANPNAASYHGNLAVLYHRWGHL |
| | GLAKKHYEISLQLDPTASGTKENYGLLRRKLELMQKKAVLE |

| | |
|---|---|
| SEQ ID NO: 129 | 1515 bp |

| | |
|---|---|
| NOV23i, | GAATTCAAATTCAATGTTCTGGAAATTGTCCAGAAGGTACTACATAAGGACAAGTCAT |
| 169252481 DNA | |
| Sequence | TAGAGAATCTCGGCATGCTCAGGAACGGGGGCCTCCTCTTCAGAATGACCCTGCTCAC |
| | CTCTGGAGGGGCTGGGATGCTCTACGTGCGCTGGAGGATCATGGGCACGCGCCCGCCG |
| | GCCTTCACCGAGGTGGACAACCCGGCCTCCTTTGCTGACAGCATGCTGGTGAGGGCCG |
| | TAAACTACAATTACTACTATTCATTGAATGCCTGGCTGCTGCTGTGTCCCTGGTGGCT |
| | GTGTTTTGATTGGTCAATGGGCTGCATCCCCCTCATTAAGTCCATCAGCGACTGGAGG |
| | GTAATTGCACTTGCAGCACTCTGGTTCTGCCTAATTGGCCTGATATGCCAAGCCCTGT |
| | GCTCTGAAGACGGCCACAAGAGAAGGATCCTTACTCTGGGCCTGGGATTTCTCGTTAT |
| | CCCATTTCTCCCCGCGAGTAACCTGTTCTTCCGAGTGGGCTTCGTGGTCGCAGAGCGT |
| | GTCCTCTACCTCCCCACCGTTGGGTACTGTGTGCTGCTGACTTTTGGATTCGGAGCCC |
| | TGAGCAAACATACCAACAAAAAGAAACTCATTGCCGCTGTCGTGCTGGGAATCTTATT |
| | CATCAACACGCTGAGATGTGTGCTGCGCAGCGGCGAGTGGCGGAGTGAGGAACAGCTT |
| | TTCAGAAGTGCTCTGTCTGTGTGTCCCCTCAATGCTAAGGTTCACTACAACATTGGCA |
| | AAAACCTGGCTGATAAAGGCAACCAGACAGCTGCCATCAGATACTACCGGGAAGCTGT |
| | AAGATTAAATCCCAAGTATGTTCATGCCATGAATAATCTTGGAAATATCTTAAAAGAA |
| | AGGAATGAGCTACAGGAAGCTGAGGAGCTGCTGTCTTTGGCTGTTCAAATACAGCCAG |
| | ACTTTGCCGCTGCGTGGATGAATCTAGGCATAGTGCAGAATAGCCTGAAACGGTTTGA |
| | AGCAGCAGAGCAAAGTTACCGGACAGCAATTAAACACAGAAGGAAATACCCAGACTGT |
| | TACTACAACCTCGGGCGTCTGTATGCAGATCTCAATCGCCACGTGGATGCCTTGAATG |
| | CGTGGAGAAATGCCACCGTGCTGAAACCAGAGCACAGCCTGGCCTGGAACAACATGAT |
| | TATACTCCTCCACAATACAGGTAATTTAGCCCAAGCTGAAGCAGTTGGAAGAGAGGCA |
| | CTGGAATTAATACCTAATGATCACTCTCTCATGTTCTCGTTCGCAAACGTGCTGGGGA |
| | AATCCCAGAAATACAAGGAATCTGAAGCTTTATTCCTCAAGCCAATTAAAGCAAATCC |
| | AAATGCTGCAAGTTACCGTGGTAATTTGGCTGTGCTTTATCATCGTTGGGGACATCTA |
| | GACTTGGCCAAGAAACACTATGAAATCTCCTCGCACCTTGACCCCACGGCATCAGGAA |
| | CTAAGGAGAATTACGGTCTGCTGAGAAGAAAGCTAGAACTAATGCAAAAGAAAGCTGT |
| | CCTCGAG |

| | |
|---|---|
| ORF Start: at 1 | ORF Stop: end of sequence |

| | | |
|---|---|---|
| SEQ ID NO: 130 | 505 aa | MW at 57221.0 kD |

| | |
|---|---|
| NOV23i, | EFKFNVLEIVQKVLHKDKSLENLGMLRNGGLLFRMTLLTSGGAGMLYVRWRIMGTCPP |
| 169252481 | |
| Protein Sequence | AFTEVDNPASFADSMLVRAVNYNYYYSLNAWLLLCPWWLCFDWSMCCIPLIKSISDWR |
| | VIALAALWFCLIGLICQALCSEDGHKRRILTLGLGFLVIPFLPASNLFFRVGFVVAER |
| | VLYLPSVGYCVLLTFGFGALSKHTKKKKLIAAVVLGILFINTLRCVLRSGEWRSEEQL |
| | FRSALSVCPLNAKVHYNIGKNLADKGNQTAAIRYYREAVRLNPKYVHANNNLGNILKE |
| | RNELQEAEELLSLAVQIQPDFAAAWMNLGIVQNSLKRFEAAEQSYRTAIKHRRKYPDC |
| | YYNLGRLYADLNRHVDALNAWRNATVLKPEHSLAWNNMIILLDNTGNLAQAEAVGREA |
| | LELIPNDHSLMFSLANVLGKSQKYKESEALFLKAIKANPNAASYRGNLAVLYHRWGHL |
| | DLAKKHYEISSQLDPTASGTKENYGLLRRKLELMQKKAVLE |

| | |
|---|---|
| SEQ ID NO: 131 | 1515 bp |

| | |
|---|---|
| NOV23j, | GAATTCAAATTCAATGTTCTGGAAATTGTCCAGAAGGTACTACATAAGGACAAGTCAT |
| 169252485 DNA | |
| Sequence | TAGAGAATCTCGGCATGCTCAGGAACGGGGGCCTCCTCTTCAGAATGACCCTGCTCAC |
| | CTCTGGAGGGGCTGGGATACTCTACGTGCGCTGGAGGATCATGGGCACGGGCCCGCCG |
| | GCCTTCACCGAGGTGGACAACCCGGCCTCCTTTGCTGACAGCATGCTGGTGAGGGCCG |
| | TAAACTACAATTACTACTATTCATTGAATGCCTGGCTCCTGCTGTGTCCCTGGTGGCT |
| | GTGTTTTGATTGGTCAATGGGCTGCATCCCCCTCATTAAGTCCATCAGCGACTGGAGG |
| | GTAATTGCACTTGCAGCACTCTCGTTCTGCCTAATTGGCCTGATATGCCAAGCCCTGT |
| | GCTCTGAAGACGGCCACAAGAGAAGGATCCTTACTCTGGGCCTGGGATTTCTCGTTAT |
| | CCCATTTCTCCCCGCGAGTAACCTGTTCTTCCGAGTGGGCTTCGTGGTCGCAGAGCGT |
| | GTCCTCTACCTCCCCAGCGTTGGGTACTGTGTGCTGCTGACTTTTGGATTCGGAGCCC |
| | TGAGCAAACATACCAAGAAAAAGAAACTCATTGCCGCTGTCGTGCTGGGAATCTTATT |
| | CATCAACACGCTGAGATGTGTGCTGCGCAGCGGCGAGTGGCGGAGTGAGGAACAGCTT |
| | TTCAGAAGTGCTCTGTCTGTGTGTCCCCTCAATGCTAAGGTTCACTACAACATTGGCA |
| | AAAACCTGGCTGATAAAGGCAACCAAACAGCTGCCATCAGATACTACCGGGAAGCTGT |
| | AAGATTAAATCCCAAGTATGTTCATGCCATGAATAATCTTGGAAATATCTTAAAAGAA |
| | AGGAATGAGCTACAGGAAGCTGAGGAGCTGCTGTCTTTGGCTGTTCAAATACAGCCAG |
| | ACTTTGCCGCTGCGTGGATGAATCTAGGCATAGTGCAGAATAGCCTGAAACGGTTTGA |
| | AGCAGCAGAGCAAAGTTACCGGACAGCAATTAAACACAGAAGGAAATACCCAGACTGT |
| | TACTACAACCTCGGGCGTCTGTATCCAGATCTCAATCGCCACGTGGATGCCTTGAATG |
| | CGTGGAGAAATGCCACCGTGCTGAAACCAGAGCACAGCCTGGCCTGGAACAACATGAT |
| | TATACTCCTCGACAATACAGGTAATTTAGCCCAAGCTGAACCAGTTGGAAGAGAGGCA |
| | CTGGAATTAATACCTAATGATCACTCTCTCATGTTCTCGTTGGCAAACGTGCTGGGGA |
| | ATCCCAGAAATACAAGGAATCTGAAGCTTTATTCCTCAAGGCAATTAAAGCAAATCC |
| | AAATGCTGCAAGTTACCATGGTAATTTGGCTGTGCTTTATCATCGTTGGGGACATCTA |
| | GACTTGGCCAAGAAACACTATGAAATCTCCTTGCAGCTTGACCCCACGGCATCAGGAA |
| | CTAAGGAGAATTACGGTCTGCTGAGAAGAAAGCTAGAACTAATGCAAAAGAAAGCTGT |
| | CCTCGAG |

| | |
|---|---|
| ORF Start: at 1 | ORF Stop: end of sequence |

| | | |
|---|---|---|
| SEQ ID NO: 132 | 505 aa | MW at 57210.0 kD |

| | |
|---|---|
| NOV23j, | EFKFNVLEIVQKVLHKDKSLENLGMLRNGGLLFRMTLLTSGGAGILYVRWRIMGTGPP |
| 169252485 | |
| Protein Sequence | AFTEVDNPASFADSMLVRAVNYNYYYSLNAWLLLCPWWLCFDWSMGCIPLIKSISDWR |
| | VIALAALWFCLIGLICQALCSEDGNKRRILTLOLGFLVIPFLPASNLFFRVGFVVAER |
| | VLYLPSVGYCVLLTFGFGALSKHTKKKKLIAAVVLGILFINTLRCVLRSGEWRSEEQL |
| | FRSALSVCPLNAKVHYNIGKNLADKGNQTAAIRYYREAVRLNPKYVHAMNNLGNILKE |
| | RNELQEAEELLSLAVQIQPDFAAAWMNLGIVQNSLKRFEAAEQSYRTAIKHRRKYPDC |
| | YYNLGRLYADLNRHVDALNAWRNATVLKPEHSLAWNNMIILLDNTGNLAQAEAVGREA |
| | LELIPNDHSLMFSLANVLGKSQKYKESEALFLKAIKANPNAASYHGNLAVLYNRWGHL |
| | DLAKKHYEISLQLDPTASGTKENYGLLRRKLELMQKKAVLE |

| | |
|---|---|
| SEQ ID NO: 133 | 1515 bp |

| | |
|---|---|
| NOV23k, | GAATTCAAATTCAATGTTCTGGAAATTGTCCAGAAGGTACTACATAAGGACAAGTCAT |
| 169252492 DNA | |
| Sequence | TAGAGAATCTCGGCATGCTCAGGAACGGGGGCCTCCTCTTCAGAATGACCCTGCTCAC |
| | CTCTGGAGGGGCTGGGATGCTCTACGTGCGCTGGAGGATCATGGGCACGGGCCCGCCG |
| | GCCTTCACCGAGGTGGACAACCCGGCCTCCTTTGCTGACAGCATGCTGGTGAGGGCCG |
| | TAAACTACAATTACTACTATTCATTGAATGCCTGGCTGCTGCTGTGTCCCTGGTGGCT |
| | GTGTTTTGATTGGTCAATGGGCTGCATCCCCCTCATTAAGTCCATCAGCGACTGGAGG |
| | GTAATTGCACTTGCAGCACTCTGGTTCTGCCTAATTGGCCTGATATGCCAAGCCCTGT |
| | GCTCTGAAGACGGCCACAAGAGAAGGATCCTTACTCTGGGCCTGGGATTTCTCGTTAT |
| | CCCATTTCTCCCCGCGAGTAACCTGTTCTTCCGAGTGGGCTTCGTGGTCGCGGAGCGT |
| | GTCCTCTACCTCCCCAGCATTGGGTACTGTGTGCTGCTGACTTTTGGATTCGGAGCCC |
| | TGAGCAAACATACCAAGAAAAAGAAACTCATTGCCGCTGTCGTGCTGGGAATCTTATT |
| | CATCAACACGCTGAGATGTGTGCTGCGCAGCGGCGAGTGGCGGAGTGAGGAACAGCTT |
| | TTCAGAAGTGCTCTGTCTGTGTGTCCCCTCAATGCTAAGGTTCACTACAACATTGGCA |
| | AAAACCTGGCTGATAAAGGCAACCAGACAGCTGCCATCAGATACTACCGGGAAGCCGT |
| | AAGATTAAATCCCAAGTATGTTCATGCCATGAATAATCTTGGAAATATCTTAAAAGAA |
| | AGGAATGAGCTACAGGAAGCTGAGGAGCTGCTGTCTTTGGCTGTTCAAATACAGCCAG |
| | ACTTTGCCGCTGCGTGGATGAATCTAGGCATAGTGCAGAATAGCCTGAAACGGTTTGA |
| | AGCAGCAGAGCAAAGTTACCGGACAGCAATTAAACACAGAAGGAAATACCCAGACTGT |
| | TACTACAACCTCGGGCGTCTGTATGCAGATCTCAATCGCCACGTGGATGCCTTGAATG |
| | CGTGGAGAAATGCCACCGTGCTGAAACCAGACCACAGCCTGGCCTGGAACAACATGAT |
| | TATACTCCTCGACAATACAGGTAATTTAGCCCAAGCTGAAGCAGTTGGAAGAGAGGCA |
| | CTGGAATTAATACCTAATGATCACTCTCTCATGTTCTCGTTGGCAAACGTGCTGGGGA |
| | AATCCCAGAAATACAAGGAATCTGAAGCTTTATTCCTCAAGGCAATTAAAGCAAATCC |
| | AAATGCTGCAAGTTACCATGGTAATTTGGCTGTGCTTTATCATCGTTGGGGACATCTA |
| | GACTTGGCCAAGAAACACTATGAAATCTCCTTGCAGCTTGACCCCACGGCATCAGGAA |
| | TAAGGAGAATTACGGTCTGCTGAGAAGGAAGCTAGAACTAATGCAAAAGAAAGCTGT |

| | |
|---|---|
| ORF Start: at 1 | ORF Stop: end of sequence |

| | | |
|---|---|---|
| SEQ ID NO: 134 | 505 aa | MW at 57242.1 kD |

| | |
|---|---|
| NOV23k, | EFKFNVLEIVQKVLHKDKSLENLGMLRNGGLLFRMTLLTSGGAGMLYVRWRIMGTGPP |
| 169252492 | |
| Protein Sequence | AFTEVDNPASFADSMLVRAVNYNYYYSLNAWLLLCPWWLCFDWSMGCIPLIKSISDWR |
| | VIALAALWFCLIGLICQALCSEDGHKRRILTLGLGFLVIPFLPASNLFFRVGFVVAER |
| | VLYLPSIGYCVLLTFGFGALSKHTKKKKLIAAVVLGILFINTLRCVLRSGEWRSEEQL |
| | FRSALSVCPLNAKVHYNIGKNLADKGNQTAAIRYYREAVRLNPKYVHAMNNLGNTLKE |
| | RNELQEAEELLSLAVQIQPDFAAAWMNLGIVQNSLKRFEAAEQSYRTAIKBRRKYPDC |
| | YYNLGRLYADLNRHVDALNAWRNATVLKPEHSLAWNNMIILLDNTGNLAQAEAVGREA |
| | LELIPNDHSLMFSLANVLGKSQKYKESEALFLKAIKANPNAASYHGNLAVLYHRWGHL |
| | DLAKKHYEISLQLDPTASGTKENYGLLRRKLELMQKKAVLE |

| | |
|---|---|
| SEQ ID NO: 135 | 1515 bp |

| | |
|---|---|
| NOV23l, | GAATTCAAATTCAATGTTCTGGAAATTGTCCAGAAGGTACTACATAAGGACAAGTCAT |
| 174104491 DNA | |
| Sequence | TACAGAATCTCGGCATGCTCAGGAACGGGGACCTCCTCTTCAGAATGACCCTGCTCAC |
| | CTCTGGAGGGGCTGGGATGCTCTACGTGCGCTGGAGGATCATGGCCACGGGCCCCCCG |
| | GCCTTCACCGAGGTGGACAACCCGGCCTCCTTTGCTGACAGCATGCTGGTGAGGGCCG |
| | TAAACTACAATTACTACTATTCATTGAATGCCTGGCTGCTGCTGTGTCCCTGGTGGCT |
| | GTGTTTTGATTGGTCAATGGGCTGCATCCCCCTCATTAAGTCCATCAGCGACTGGAGG |
| | GTAATTGCACTTGCAGCACTCTGGTTCTGCCTAATTGGCCTGATATGCCAAGCCCTGT |
| | GCTCTGAAGACGGCCACAAGAGAAGGATCCTTACTCTGGGCCTGGGATTTCTCGTTAT |
| | CCCATTTCTCCCCGCGAGTAACCTGTTCTTCCGAGTGGGCTTCGTGGTCGCGGAGCGT |
| | GTCCTCTACCTCCCCAGCATTGGGTACTGTGTGCTGCTGACTTTTGGATTCGGAGCCC |
| | TGAGCAAACATACCAAGAAAAAGAAACTCATTGCCGCTGTCGTGCTGGGAATCTTATT |
| | CATCAACACGCTGAGATGTGTGCTGCGCAGCGGCGAGTGGCGGAGTGAGGAACAGCTT |
| | TTCAGAAGTCCTCTGTCTGTGTGTCCCCTCAATGCTAAGGTTCACTACAACATTGGCA |
| | AAAACCTGGCTGATAAAGGCAACCAGACAGCTGCCATCAGATACTACCGGGAAGCTGT |
| | AAGATTAAATCCCAAGTATGTTCATGCCATGAATAATCTTGGAAATATCTTAAAAGAA |
| | AGGAATGAGCTACAGGAAGCTGAGGAGCTGCTGTCTTTGGCTGTTCAAATACAGCCAG |
| | ACTTTGCCGCTGCGTGGATGAATCTAGGCATAGTGCAGAATAGCCTGAAACGGTTTGA |
| | AGCAGCAGAGCAAAGTTACCGGACAGCAATTAAACACAGAAGGAAATACCCAGACTGT |
| | TACTACAACCTCGGGCGTCTGTATGCAGATCTCAATCGCCACGTCGATGCCTTGAATG |
| | CGTGGAGAAATGCCACCGTGCTGAAACCAGACCACAGCCTGGCCTGGAACAACATGAT |
| | TATACTCCTCGACAATACAGGTAATTTAGCCCAAGCTGAAGCAGTTGGAAGAGAGGCA |
| | CTGGAATTAATACCTAATGATCACTCTCTCATGTTCTCGTTGGCAAACGTGCTGGGGA |
| | AATCCCAGAAATACAAGGAATCTGAAGCTTTATTCCTCAAGGCAATTAAAGCAAATCC |
| | AAATGCTGCAAGTTACCATGGTAATTTGGCTGTGCTTTATCATCGTTGGGGACATCTA |
| | GACTTGGCCAAGAAACACTATGAAATCTCCTTGCAGCTTGACCCCACGGCATCAGGAA |
| | CTAAGGAGAATTACGGTCTGCTGAGAAGAAAGCTAGAACTAATGCAAAAGAAAGCTGT |

| | |
|---|---|
| ORF Start: at 1 | ORF Stop: end of sequence |

| | | |
|---|---|---|
| SEQ ID NO: 136 | 505 aa | MW at 57300.1 kD |

| | |
|---|---|
| NOV23l, | EFKFNVLEIVQKVLHKDKSLENLGMLRNGDLLFRMTLLTSGGAGMLYVRWRIMGTGPP |
| 174104491 | |
| Protein Sequence | AFTEVDNPASFADSMLVRAVNYNYYYSLNAWLLLCPWWLCFDWSMGCIPLIKSISDWR |
| | VIALAALWFCLICLICQALCSEDGHKRRILTLGLGFLVIPFLPASNLFFRVGFVVAER |
| | VLYLPSIGYCVLLTFGFGALSKHTKKKKLIAAVVLGILFINTLRCVLRSGEWRSEEQL |
| | FRSALSVCPLNAKVHYNIGKNLADKGNQTAAIRYYREAVRLNPKYVHAMNNLGNILKE |
| | RNELQEAEELLSLAVQIQPDFAAAWMNLGIVQNSLKRFEAAEQSYRTAIKHRRKYPDC |
| | YYNLGRLYADLNRHVDALNAWRNATVLKPEHSLAWNNMIILLDNTGNLAQAEAVGREA |
| | LELIPNDHSLMFSLANVLGKSQKYKESEALFLKAIKANPNAASYHGNLAVLYHRWGHL |
| | DLAKKHYEISLQLDPTASGTKENYGLLRRKLELMQKKAVLE |

| | |
|---|---|
| SEQ ID NO: 137 | 855 bp |

| | |
|---|---|
| NOV23m, | GAATTCAGCGGCGAGTGGCGGAGTGAGGAACAGCTTTTCAGAAGTGCTCTGTCTGTGT |
| 169252509 DNA | |
| Sequence | GTCCCCTCAATGCTAAGGTTCACTACAACATTGGCAAAAACCTGGCTGATAAAGGCAG |
| | CCAGACAGCTGCCATCAGATACTACCGGGAAGCTGTAAGATTAAATCCCAAGTATGTT |
| | CATGCCATGAATAATCTTGGAAATATCTTAAAAGAAAGGAATGAGCTACAGGAAGCTG |
| | AGGAGCTGCTGTCTTTGGCTGTTCAAATACAGCCAGACTTTGCCGCTGCGTGGATGAA |
| | TCTAGGCATAGTGCAGAATAGCCTGAAACGGTTTGAAGCAGCAGAGCAAAGTTATCCG |
| | ACAGCAATTAAACACAGAAGGAAATACCCAGACTGTTACTACAACCTCGGGCGTCTGT |
| | ATGCAGATCTCAATCGCCACGTGGATGCCTTGAATGCGTGGAGAAATGCCACCGTGCT |
| | GAAACCAGAGCACAGCCTGGCCTGGAACAACATGATTATACTCCTCGACAATACAGGT |
| | AATTTAGCCCAAGCTGAAGCAGTTGGAAGAGAGGCACTGGAATTAATACCTAATGATC |
| | ACTCTCTCATGTTCTCGTTGGCAAACGTGCTGGGGAAATCCCAGAAATACAAGGAATC |
| | TGAAGCTTTATTCCTCAAGGCAATTAAAGCAAATCCAAATGCTGCAAGTTACCATGGT |
| | AATTTGGCTGTGCTTTATCATCGTTGGGGACATCTAGACTTGGCCAAGAAACACTATG |
| | AAATCTCCTTGCAGCTTGACCCCACGGCATCAGGAACTAAGGAGAATTACGGTCTGCT |
| | GAGAAGAAAGCTAGAACTAATGCAAAAGAAAGCTGTCCTCGAG |

| | |
|---|---|
| ORF Start: at 1 | ORF Stop: end of sequence |

| | | |
|---|---|---|
| SEQ ID NO: 138 | 285 aa | MW at 32488.7 kD |

| | |
|---|---|
| NOV23m, | EFSGEWRSEEQLFRSALSVCPLNAKVHYNIGKNLADKGSQTAAIRYYREAVRLNPKYV |
| 169252509 | |
| Protein Sequence | HAMNNLGNILKERNELQEAEELLSLAVQIQPDFAAAWMNLGIVQNSLKRFEAAEQSYR |
| | TAIKHRRKYPDCYYNLGRLYADLNRHVDALNAWRNATVLKPEHSLAWNNMIILLDNTG |
| | NLAQAEAVGREALELIPNDHSLMFSLANVLGKSQKYKESEALFLKAIKANPNAASYHG |
| | NLAVLYHRWGHLDLAKKHYEISLQLDPTASGTKENYGLLRRKLELMQKKAVLE |

| | |
|---|---|
| SEQ ID NO: 139 | 855 bp |

| | |
|---|---|
| NOV23n, | GAATTCAGCGGCGAGTGGCGGAGTGAGGAACAGCTTTTCAGAAGTGCTCTGTCTGTGT |
| 169252515 DNA | |
| Sequence | GTCCCCTCAATGCTAAGGTTCACCACAACATTGGCAAAAACCTGGCTGATAAAGGCAA |
| | CCAGACAGCTGCCATCAGATACTACCGGGAAGCTGTAAGATTAAATCCCAAGTATGTT |
| | CATGCCATGAATAATCTTGGAAATATCTTAAAAGAAAGGAATGAGCTACAGGAAGCTG |
| | AGGAGCTGCTGTCTTTGGCTGTTCAAATACAGCCAGACTTTGCCGCTGCGTGGATGAA |
| | TCTAGGCATAGTGCAGAATAGCCTGAAACGGTTTGAAGCAGCAGAGCAAAGTTACCGG |
| | ACAGCAATTAAACACAGAAGGAAATACCCAGACTGTTACTACAACCTCGGGCGTCTGT |
| | ATGCAGATCTCAATCGCCACGTGGATGCCTTGAATGCGTGGAGAAATGCCACCGTGCT |
| | GAAACCAGAGCACAGCCTGGCCTGGAACAACATGATTATACTCCTCGACAATACAGGT |
| | AATTTAGCCCAAGCTGAAGCAGTTGGAAGAGAGGCACTGGAATTAATACCTAATGATC |
| | ACTCTCTCATGTTCTCGTTGGCAAACGTGCTGGGGAAATCCCAGAAATACAAGGAATC |
| | TGAAGCTTTATTCCTCAAGGCAATTAAAGCAAATCCAAATGCTGCAAGTTACCATGGT |
| | AATTTGGCTGTGCTTTATCATCGTTGGGGACATCTAGACTTGGCCAAGAAACACTATC |
| | AAATCTCCTTGCAGCTTGACCCCACGGCATCAGGAACTAAGGAGAATTACGGTCTCCT |
| | GAGAAGAAAGCTAGAACTAATGCAAAAGAAAGCTGTCCTCGAG |

| | |
|---|---|
| ORF Start: at 1 | ORF Stop: end of sequence |

| | | |
|---|---|---|
| SEQ ID NO: 140 | 285 aa | MW at 32489.7 kD |

| | |
|---|---|
| NOV23n, | EFSGEWRSEEQLFRSALSVCPLNAKVHHNIGKNLADKGNQTAAIRYYREAVRLNPKYV |
| 169252515 | |
| Protein Sequence | HAMNNLGNILKERNELQEAEELLSLAVQIQPDFAAAWMNLCIVQNSLKRFEAAEQSYR |
| | TAIKHRRKYPDCYYNLGRLYADLNRHVDALNAWRNATVLKPEHSLAWNNMIILLDNTG |
| | NLAQAEAVGREALELIPNDHSLMFSLANVLGKSQKYKESEALFLKAIKANPNAASYHG |
| | NLAVLYHRWGHLDLAKKHYEISLQLDPTASGTKENYGLLRRKLELMQKKAVLE |

| | |
|---|---|
| SEQ ID NO: 141 | 855 bp |

| | |
|---|---|
| NOV23o, | GAATTCAGCGGCGAGTGGCGGAGTGAGGAACAGCTTTTCAGAAGTGCTCTGTCTGTGT |
| 169252519 DNA | |
| Sequence | GTCCCCTCAATGCTAAGGTTCACTACAACATTGGCAAAAACCTGGCTGATAAAGGCAA |
| | CCAGACAGCCGCCATCAGATACTACCGGCAAGCTGTAAGATTAAATCCCAAGTATGTT |
| | CATGCCATGAATAATCTTGGAAATATCTTAAAAGAAAGGAATGAGCTACAGGAAGCTG |
| | AGGACCTGCTGTCTTTGGCTGTTCAAATACAGCCAGACTTTGCCGCTGCGTGGATGAA |
| | TCTAGGCATAGTGCAGAATAGCCTGAAACGGTTTGAAGCAGCAGAGCAAAGTTACCGG |
| | ACAGCAATTAAACACAGAAGGAAATACCCAGACTGTTACTACAACCTCGGGCGTCTGT |
| | ATGCAGATCTCAATCGCCACGTGGATGCCTTGAATGCGTGGAGAAATGCCACCGTGCT |
| | GAAACCAGAGCACAGCCTGGCCTGGAACAACATGATTATACTCCTCGACAATACAGGT |
| | AATTTAGCCCAAGCTGAAGCAGTTGGAAGAGAGGCACTGGAATTAATACCTAATGATC |
| | ACTCTCTCATGTTCTCGTTGGCAAACGTGCTGGGGAAATCCCAGAAATACAAAGAATC |
| | TGAAGCTTTATTCCTCAAGGCAATTAAAGCAAATCCAAATGCTGCAAGTTACCATGGT |
| | AATTTGGCTGTGCTTTATCATCGTTGGGGACATCTAGACTTGGCCAAGAAACACTATG |
| | AAATCTCCTTGCAGCTTGACCCCACGGCATCAGGAACTAAGGAGAATTACGGTCTGCT |
| | ACAAGAAAGCTAGAACTAATGCAAAAGAAAGCTGTCCTCGAG |

| | |
|---|---|
| ORF Start: at 1 | ORF Stop: end of sequence |

| | | |
|---|---|---|
| SEQ ID NO: 142 | 285 aa | MW at 32515.7 kD |

| | |
|---|---|
| NOV23o, | EFSGEWRSEEQLFRSALSVCPLNAKVHYNIGKNLADKGNQTAAIRYYREAVRLNPKYV |
| 169252519 | |
| Protein Sequence | HAMNNLGNILKERNELQEAEELLSLAVQIQPDFAAAWMNLGIVQNSLKRFEAAEQSYR |
| | TAIKHRRKYPDCYYNLGRLYADLNRHVDALNAWRNATVLKPEHSLAWNNMIILLDNTG |
| | NLAQAEAVGREALELIPNDHSLMFSLANVLGKSQKYKESEALFLKAIKANPNAASYHG |
| | NLAVLYHRWGHLDLAKKHYEISLQLDPTASGTKENYGLLRRKLELMQKKAVLE |

| | |
|---|---|
| SEQ ID NO: 143 | 855 bp |

| | |
|---|---|
| NOV23p, | GAATTCAGCGGCGAGTGGCGGAGTGAGGAACAGCTTTTCAGAAGTGCTCTGTCTGTGT |
| 169252524 DNA | |
| Sequence | GTCCCCTCAATGCTAAGGTTCACTACAACATTGGCAAAAACCTGGCTGATAAAGGCAA |
| | CCAGACAGCTGCCATCAGATACTACCGGGAAGCTGTAAGATTAAATCCCAAGTATGTT |
| | CATGCCATGAATAATCTTGGAAATATCTTAAAAGAAAGGAATGAGCTACAGGAAGTTG |
| | AGGAGCTGCTGTCTTTGGCTGTTCAAATACAGCCAGACTTTGCCGCTGCGTGGATGAA |
| | TCTAGGCATAGTGCAGAATAGCCTGAAACGCTTTGAAGCAGCAGAGCAAAGTTACCGG |
| | ACAGCAATTAAACACAGAAGGAAATACCCAGACTGTTACTACAACCTCGGCCGTCTGT |
| | ATGCAGATCTCAATCGCCACGTGCATGCCTTGAATGCGTGGAGAAATGCCACCGTGCT |
| | GAAACCAGAGCACAGCCTGGCCTGGAACAACATGATTATACTCCTCCACAATACAGGT |
| | AATTTAGCCCAAGCTGAAGCAGTTGGAAGAGAGGCACTGGAATTAATACCTAATGATC |
| | ACTCTCTCATGTTCTCGTTGGCAAACGTGCTGGGGAAATCCCAGAAATACAAGGAATC |
| | TGAAGCTTTATTCCTCAAGGCAATTAAAGCAAATCCAAATCCTGCAAGTTACCATGGT |
| | AATTTGGCTGTGCTTTATCATCGTTGGGGACATCTAGACTTGGCCAAGAAACACTATG |
| | AAATCTCCTTGCAGCTTGACCCCACGGCATCAGGAACTAACGAGAATTACGGTCTGCT |
| | GAGAAGAAAGCTAGAACTAATGCAAAAGAAAGCTGTCCTCGAG |

| | |
|---|---|
| ORF Start: at 1 | ORF Stop: end of sequence |

| | | |
|---|---|---|
| SEQ ID NO: 144 | 285 aa | MW at 32543.8 kD |

| | |
|---|---|
| NOV23p, | EFSGEWRSEEQLFRSALSVCPLNAKVIIYNIGKNLADKCNQTAAIRYYREAVRLNPKYV |
| 169252524 | |
| Protein Sequence | HAMNNLGNILKERNELQEVEELLSLAVQIQPDFAAAWMNLGIVQNSLKRFEAAEQSYR |
| | TAIKHRRKYPDCYYNLGRLYADLNRHVDALNAWRNATVLKPEHSLAWNNMIILLDNTG |
| | NLAQAEAVGREALELIPNDHSLMFSLANVLGKSQKYKESEALFLKAIKANPNAASYHG |
| | NLAVLYHRWGHLDLAKKHYEISLQLDPTASGTKENYGLLRRKLELMQKKAVLE |

| | |
|---|---|
| SEQ ID NO: 145 | 855 bp |

| | |
|---|---|
| NOV23q, | GAATTCAGCGGCGAGTGGCGGAGTGAGGAACAGCTTTTCAGAAGTCCTCTGTCTGTGT |
| 169252528 DNA | |
| Sequence | GTCCCCTCAATGCTAAGGTTCACTACAACATTGGCAAAAACCTGGCTGATAAAGGCAA |
| | CCAGACAGCTGCCATCAGATACTACCGGGAAGCTGTAAGATTAAATCCCAAGTATGTT |
| | CATGCCATGAATAATCTTGGAAATATCTTAAAAGAAAGGAATGAGCTACAGGAAGCTG |
| | AGGAGCTGCTGTCTTTGGCTGTTCAAATACAGCCAGACTTTGCCGCTGCGTGGATGAA |
| | TCTAGGCATAGTGCAGAATAGCCTGAAACGGTTTGAAGCAGCAGAGCAAAGTTACCGG |
| | ACAGCAATTAAACACAGAAGGAAATACCCAGACTGTTACTACAACCTCGGGCGTCTGT |
| | ATGCAGATCTCAATCGCCACGTGGATGCCTTGAATGCGTGGAGAAATGCCACCGTGCT |
| | GAAACCAGAGCACAGCCTGGCCTGGAACAACATGATTATACTCCTCGACAATACAGGT |
| | AATTTAGCCCAAGCTGAAGCAGTTGGAAGAGAGGCACTGGAATTAATACCTAATGATC |
| | ACTCTCTCATGTTCTCGTTGGCAAACGTGCTGGGGAAATCCCAGAAATACAAGGAATC |
| | TGAAGCTTTATCCCTCAAGGCAATTAAAGCAAATCCAAATGCTGCAAGTTACCATGGT |
| | AATTTGGCTGTGCTTTATCATCGTTGGGGACATCTAGACTTGGCCAAGAAACACTATG |
| | AAATCTCCTTGCAGCTTGACCCCACGGCATCAGGAACTAAGGAGAATTACGGTCTGCT |
| | AGAAGAAAGCTAGAACTAATGCAAAAGAAAGCTGTCCTCGAG |

| | |
|---|---|
| ORF Start: at 1 | ORF Stop: end of sequence |

| | | |
|---|---|---|
| SEQ ID NO: 146 | 285 aa | MW at 32455.6 kD |

| | |
|---|---|
| NOV23q, | EFSGEWRSEEQLFRSALSVCPLNAKVHYNIGKNLADKGNQTAAIRYYREAVRLNPKYV |
| 169252528 | |
| Protein Sequence | HAMNNLGNILKERNELQEAEELLSLAVQIQPDFAAAWMNLGIVQNSLKRFEAAEQSYR |
| | TAIKHRRKYPDCYYNLGRLYADLNRHVDALNAWRNATVLKPEHSLAWNNMIILLDNTG |
| | NLAQAEAVGREALELIPNDHSLMFSLANVLGKSQKYKESEALSLKAIKANPNAASYHG |
| | NLAVLYHRWGHLDLAKKHYEISLQLDPTASGTKENYGLLRRKLELMQKKAVLE |

| | |
|---|---|
| SEQ ID NO: 147 | 855 bp |

| | |
|---|---|
| NOV23r, | GAATTCAGCGGCGAGTGGCGGAGTGAGGAACAGCTTTTCAGAAGTGCTCTGTCTGTGT |
| 169252547 DNA | |
| Sequence | GTCCCCTCAATGCTAAGGTTCACTACAACATTGGCAAAAACCTGGCTGATAAAGGCAA |
| | CCAGACAGCTGCCATCAGATACTACCGGGAAGCTGTAAGATTAAATCCCAAGTATGTT |
| | CATGCCATGAATAATCTTGGAAATATCTTAAAAGAAAGGAATGAGCTACAGGAAGCTG |
| | AGGAGCTGCTGTCTTTGGCTGTTCAAATACACCCAGACTTTGCCGCTGCGTGGATGAA |
| | TCTAGGCATAGTGCAGAATAGCCTGAAACGGTTTGAAGCAGCAGAGCAAAGTTACCGG |
| | ACAGCAATTAAACACAGAAGGAAATACCCAGACTGTTACTACAACCTCGGGCGTCTGT |
| | ATGCAGATCTCAATCGCCACGTGGATGCCTTGAATGCGTGGAGAAATGCCACCGTGCT |
| | GAAACCAGAGCACAGCCTGGCCTGGAACAACATGATTATACTCCTCGACAATACAGGT |
| | AATTTAGCCCAAGCTGAACCAGTTGGAAGAGAGGCACTGCAATTAATACCTAATGATC |
| | ACTCTCTCATGTTCTCGTTGGCAAACGTGCTGGGGAAATCCCAGAAATACAAGGAATC |
| | TGAAGCTTTATTCCTCAAGGCAATTAAAGCAAATCCAAATGCTGCAAGTTACCATGGT |
| | AATTTGGCTGTGCTTTATCATCGTTGGGGACATCTAGACTTGGCCAAGAAACACCATG |
| | AAATCTCCTTGCAGCTTGACCCCACGGCATCACGAACTAAGGAGAATTACGGTCTGCT |
| | GAGAAGAAAGCTAGAACTAATGCAAAACAAAGCTGTCCTCGAG |

| | |
|---|---|
| ORF Start: at 1 | ORF Stop: end of sequence |

| | | |
|---|---|---|
| SEQ ID NO: 148 | 285 aa | MW at 32489.7 kD |

| | |
|---|---|
| NOV23r, | EFSGEWRSEEQLFRSALSVCPLNAKVHYNIGKNLADKGNQTAAIRYYREAVRLNPKYV |
| 169252547 | |
| Protein Sequence | HAMNNLGNILKERNELQEAEELLSLAVQIQPDFAAAWMNLGIVQNSLKRFEAAEQSYR |
| | TAIKHRRKYPDCYYNLGRLYADLNRHVDALNAWRNATVLKPEHSLAWNNMIILLDNTG |
| | NLAQAEAVGREALELIPNDHSLMFSLANVLGKSQKYKESEALFLKAIKANPNAASYHG |
| | NLAVLYHRWGHLDLAKKHHEISLQLDPTASGTKENYGLLRRKLELMQKKAVLE |

| | |
|---|---|
| SEQ ID NO: 149 | 855 bp |

| | |
|---|---|
| NOV23s, | GAATTCAGCGGCGAGTGGCGGAGTGAGGAACAGCTTTTCAGAAGTGCTCTGTCTGTGT |
| 169252557 DNA | |
| Sequence | GTCCCCTCAATGCTAAGGTTCACTACAACATTGGCAAAAACCTGGCTGATAAAGGCAA |
| | CCAGACAGCTGCCATCAGATACTACCGGGAAGCTGTAAGATTAAATCCCAAGTATGTT |
| | CATGCCATGAATAATCTTGGAAATATCTTAAAAGAAAGGAATGAGCTACAGGAAGCTG |
| | AGGAGCTGCTGTCTTTCGCTGTTCAAATACAGCCAGACTTTGCCGCTGCGTGGATGAA |
| | TCTAGGCATAGTGCAGAATAGCCTGAAACGGTTTGAAGCAGCAGAGCAAAGTTACCGG |
| | ACAGCAATTAAACACAGAAGGAAATACCCAGACTGTTACTACAACCTCGGGCGTCTGT |
| | ATGCAGATCTCAATCGCCACGTGGATGCCTTGAATGCGTGGAGAAATGCCACCGTGCT |
| | GAAACCAGAGCACAGCCTGGCCTGGAACAACATGATTATACTCCTCGACAATACAGGT |
| | AATTTAGCCCAAGCTGAAGCAGTTGGAAGAGAGGCACTGGAATTAATACCTAATGATC |
| | ACTCTCTCATGTTCTCGTTGGCAAACGTGCTGGGGAAATCCCAGAAATACAGGGAATC |
| | TGAAGCTTTATTCCTCAAGGCAATTAAAGCAAATCCAAATGCTGCAAGTTACCATGGT |
| | AATTTGGCTGTGCTTTATCATCGTTGGGGACATCTAGACTTGGCCAAGAAACACTATG |
| | AAATCTCCTTGCAGCTTCACCCCACGGCATCAGGAACTAAGGACAATTACGGTCTGCT |
| | GAGAAGAAAGCTAGAACTAATGCAAAAGAAAGCTGTCCTCGAG |

| | |
|---|---|
| ORF Start: at 1 | ORF Stop: end of sequence |

| | | |
|---|---|---|
| SEQ ID NO: 150 | 285 aa | MW at 32543.7 kD |

| | |
|---|---|
| NOV23s, | EFSGEWRSEEQLFRSALSVCPLNAKVHYNIGKNLADKGNQTAAIRYYREAVRLNPKYV |
| 169252557 | |
| Protein Sequence | HANNNLGNILKERNELQEAEELLSLAVQIQPDFAAAWMNLGIVQNSLKRFEAAEQSYR |
| | TAIKHRRKYPDCYYNLGRLYADLNRHVDALNAWRNATVLKPEHSLAWNNMIILLDNTG |
| | NLAVLYHRWGHLDLAKKHYEISLQLDPTASGTKENYGLLRRKLELMQKKAVLE |

| | |
|---|---|
| SEQ ID NO: 151 | 1515 bp |

| | |
|---|---|
| NOV23t, | GAATTCAAATTCAATGTTCTGGAAATTGTCCAGAAGGTACTACATAAGGACAAGTCAT |
| 174104491 DNA | |
| Sequence | TAGAGAATCTCGGCATGCTCAGGAACGGGGACCTCCTCTTCAGAATGACCCTGCTCAC |
| | CTCTGGAGGGGCTCGGATGCTCTACGTGCGCTGGAGGATCATGGGCACGGGCCCGCCG |
| | GCCTTCACCGAGGTGGACAACCCGGCCTCCTTTGCTGACAGCATGCTGGTGAGGGCCG |
| | TAAACTACAATTACTACTATTCATTGAATGCCTGGCTGCTGCTGTGTCCCTGGTGGCT |
| | GTGTTTTGATTGGTCAATGGGCTGCATCCCCCTCATTAAGTCCATCAGCGACTGGAGG |
| | GTAATTGCACTTGCAGCACTCTGGTTCTGCCTAATTGGCCTGATATGCCAAGCCCTGT |
| | GCTCTGAAGACGGCCACAAGAGAACGATCCTTACTCTGGGCCTGGGATTTCTCGTTAT |
| | CCCATTTCTCCCCGCGAGTAACCTGTTCTTCCGAGTGGGCTTCGTGGTCGCGGAGCGT |
| | GTCCTCTACCTCCCCAGCATTCGGTACTGTGTGCTGCTGACTTTTGGATTCGGAGCCC |
| | TGAGCAAACATACCAAGAAAAAGAAACTCATTGCCGCTGTCGTGCTGGGAATCTTATT |
| | CATCAACACGCTGAGATGTGTGCTGCGCAGCGGCGAGTGGCGGAGTGAGGAACAGCTT |
| | TTCAGAAGTGCTCTGTCTGTGTGTCCCCTCAATGCTAAGGTTCACTACAACATTGGCA |
| | AAAACCTGGCTGATAAAGGCAACCAGACAGCTGCCATCAGATACTACCGGGAAGCTGT |
| | AAGATTAAATCCCAAGTATGTTCATGCCATGAATAATCTTGGAAATATCTTAAAAGAA |
| | AGGAATGAGCTACAGGAAGCTGAGGAGCTGGCTGTCTTTGGCTGTTCAAATACAGCCAG |
| | ACTTTGCCCCTGCGTGGATGAATCTAGGCATAGTGCAGAATAGCCTGAAACGGTTTGA |
| | AGCAGCAGAGCAAAGTTACCGGACAGCAATTAAACACAGAACGAAATACCCAGACTGT |
| | TACTACAACCTCGGGCGTCTGTATGCAGATCTCAATCGCCACGTGGATGCCTTGAATG |
| | CGTGGAGAAATGCCACCGTGCTGAAACCAGAGCACAGCCTGGCCTGGAACAACATGAT |
| | TATACTCCTCGACAATACAGGTAATTTAGCCCAAGCTGAAGCAGTTGGAAGAGACGCA |
| | CTGGAATTAATACCTAATGATCACTCTCTCATGTTCTCGTTGGCAAACGTCCTGGGGA |
| | AATCCCAGAAATACAAGGAATCTGAAGCTTTATTCCTCAAGGCAATTAAAGCAAATCC |
| | AAATGCTGCAAGTTACCATGGTAATTTGGCTGTGCTTTATCATCGTTGGGGACATCTA |
| | GACTTGGCCAAGAAACACTATGAAATCTCCTTGCAGCTTGACCCCACCGCATCAGGAA |
| | CTAAGGAGAATTACGGTCTGCTGAGAAGAAAGCTAGAACTAATGCAAAAGAAAGCTGT |

| | |
|---|---|
| ORF Start: at 1 | ORF Stop: end of sequence |

| | | |
|---|---|---|
| SEQ ID NO: 152 | 505 aa | MW at 57300.1 kD |

| | |
|---|---|
| NOV23t, | EFKFNVLEIVQKVLHKDKSLENLGMLRNGDLLFRMTLLTSGGAGMLYVRWRIMGTGPP |
| 174104491 | |
| Protein Sequence | AFTEVDNPASFADSMLVRAVNYNYYYSLNAWLLLCPWWLCFDWSMGCIPLIKSISDWR |
| | VIALAALWFCLIGLICQALCSEDGHKRRILTLGLGFLVIPFLPASNLFFRVGFVVAER |
| | VLYLPSIGYCVLLTFGFGALSKHTKKKKLIAAVVLGILFINTLRCVLRSGEWRSEEQL |
| | FRSALSVCPLNAKVHYNIGKNLADKGNQTAAIRYYREAVRLNPKYVHANNNLGNILKE |
| | RNELQEAEELLSLAVQIQPDFAAAWMNLGIVQNSLKRFEAAEQSYRTAIKHRRKYPDC |
| | YYNLGRLYADLNRHVDALNAWRNATVLKPEHSLAWNNMIILLDNTGNLAQAEAVGREA |
| | LELIPNDHSLMFSLANVLGKSQKYKESEALFLKAIKANPNAASYHGNLAVLYHRWGHL |
| | DLAKKHYEISLQLDPTASGTKENYGLLRRKLELMQKKAVLE |

| | |
|---|---|
| SEQ ID NO: 153 | 843 bp |

| | |
|---|---|
| NOV23u, | AGCGGCGAGTGGCGGAGTGAGGAACAGCTTTTCAGAAGTGCTCTGTCTGTGTGTCCCC |
| CG57774-02 DNA | |
| Sequence | TCAATGCTAAGGTTCACTACAACATTGGCAAAAACCTGGCTGATAAAGGCAACCAGAC |
| | AGCTGCCATCAGATACTACCGGGAAGCTGTAAGATTAAATCCCAAGTATGTTCATGCC |
| | ATGAATAATCTTGGAAATATCTTAAAAGAAAGGAATGAGCTACAGGAAGCTGAGGAGC |
| | TGCTGTCTTTGGCTGTTCAAATACAGCCAGACTTTGCCGCTGCGTGGATCAATCTAGG |
| | CATAGTGCAGAATAGCCTGAAACGGTTTGAAGCAGCAGAGCAAAGTTACCGGACAGCA |
| | ATTAAACACACAAGGAAATACCCAGACTGTTACTACAACCTCGGGCGTCTGTATGCAG |
| | ATCTCAATCGCCACGTGGATGCCTTCAATGCGTGGAGAAATGCCACCGTGCTGAAACC |
| | AGAGCACAGCCTGGCCTGGAACAACATGATTATACTCCTCCACAATACAGGTAATTTA |
| | GCCCAAGCTGAAGCAGTTGGAAGAGAGGCACTGGAATTAATACCTAATGATCACTCTC |
| | TCATGTTCTCGTTGGCAAACGTGCTGGGGAAATCCCAGAAATACAAGGAATCTGAAGC |
| | TTTATTCCTCAAGGCAATTAAAGCAAATCCAAATGCTGCAAGTTACCATGGTAATTTG |
| | GCTGTGCTTTATCATCGTTGGGGACATCTAGACTTGGCCAAGAAACACTATGAAATCT |
| | CCTTGCAGCTTGACCCCACGGCATCAGGAACTAAGGAGAATTACGGTCTGCTGAGAAG |
| | AAAGCTAGAACTAATGCAAAAGAAAGCTGTC |

| | |
|---|---|
| ORF Start: at 1 | ORF Stop: end of sequence |

| | | |
|---|---|---|
| SEQ ID NO: 154 | 281 aa | MW at 31997.2 kD |

| | |
|---|---|
| NOV23u, | SGEWRSEEQLFRSALSVCPLNAKVHYNIGKNLADKGNQTAAIRYYREAVRLNPKYVHA |
| CG57774-02 | |
| Protein Sequence | MNNLGNILKERNELQEAEELLSLAVQIQPDFAAAWMNLGIVQNSLKRFEAAEQSYRTA |
| | IKHRRKYPDCYYNLGRLYADLNRHVDALNAWRNATVLKPEHSLAWNNMIILLDNTGNL |
| | AQAEAVGREALELIPNDHSLMFSLANVLGKSQKYKESEALFLKAIKANPNAASYHGNL |
| | AVLYHRWGHLDLAKKHYEISLQLDPTASGTKENYGLLRRKLELMQKKAV |

| | |
|---|---|
| SEQ ID NO: 155 | 1503 bp |

| | |
|---|---|
| NOV23v, | AAATTCAATGTTCTGGAAATTGTCCAGAAGGTACTACATAAGGACAAGTCATTAGAGA |
| CG57774-03 DNA | |
| Sequence | ATCTCGGCATGCTCAGGAACGGGGGCCTCCTCTTCAGAATGACCCTGCTCACCTCTGG |
| | AGGGGCTGGGATGCTCTACGTGCGCTGGAGGATCATGGGCACGGGCCCGCCGGCCTTC |
| | ACCGAGGTGGACAACCCGGCCTCCTTTGCTGACAGCATGCTGGTGAGGGCCGTAAACT |
| | ACAATTACTACTATTCATTGAATGCCTGGCTGCTGCTGTGTCCCTGGTGGCTGTGTTT |
| | TGATTGGTCAATGGGCTGCATCCCCCTCATTAAGTCCATCAGCGACTGGAGGGTAATT |
| | GCACTTGCAGCACTCTGGTTCTGCCTAATTGGCCTGATATGCCAAGCCCTCTGCTCTG |
| | AAGACGGCCACAAGAGAAGGATCCTTACTCTGGGCCTGGGATTTCTCGTTATCCCATT |
| | TCTCCCTCCGAGTAACCTGTTCTTCCGAGTGGGCTTCGTGGTCGCGGAGCGTGTCCTC |
| | TACCTCCCCAGCGTTGGGTACTGTGTGCTGCTGACTTTTGGATTCGGAGCCCTGAGCA |
| | AACATACCAAGAAAAAGAAACTCATTGCCGCTGTCGTCCTGGGAATCTTATTCATCAA |
| | CACGCTGAGATGTGTGCTGCGCAGCGGCGAGTGGCGGAGTGAGGAACAGCTTTTCAGA |
| | AGTGCTCTGTCTGTGTGTCCCCTCAATGCTAAGGTTCACTACAACATTGGCAAAAACC |
| | TGGCTGATAAAGGCAACCACACACCTGCCATCAGATACTACCGGGAAGCTGTAAGATT |
| | AAATCCCAAGTATGTTCATCCCATGAATAATCTTGGAAATATCTTAAAAGAAAGGAAT |
| | GAGCTACAGGAAGCTGAGGAGCTGCTGTCTTTGGCTGTTCAAATACAGCCAGACTTTG |
| | CCGCTGCGTGGATCAATCTAGGCATAGTGCAGAATAGCCTGAAACCGTTTGAAGCAGC |
| | AGAGCAAAGTTACCCGACAGCAATTAAACACAGAAGGAAATACCCAGACTGTTACTAC |
| | AACCTCGGGCGTCTGTATGCAGATCTCAATCGCCACGTGGATGCCTTGAATGCGTGGA |
| | GAAATGCCACCGTCCTGAAACCAGAGCACAGCCTGGCCTGGAACAACATGATTATACT |
| | CCTCGACAATACAGGTAATTTAGCCCAAGCTGAAGCAGTTCGAAGAGAGGCACTGGAA |
| | TTAATACCTAATGATCACTCTCTCATGTTCTCGTTGGCAAACGTGCTGGGGAAATCCC |
| | AGAAATACAAGGAATCTGAAGCTTTATTCCTCAAGGCAATTAAAGCAAATCCAAATGC |
| | TGCAAGTTACCATGGTAATTTGGCTGTGCTTTATCATCGTTGGGGACATCTAGACTTG |
| | GCCAAGAAACACTATGAAATCTCCTTGCAGCTTGACCCCACGGCATCAGGAACTAAGG |
| | AGAATTACGGTCTGCTGAGAAGAAAGCTAGAACTAATGCAAAAGAAAGCTGTC |

| | |
|---|---|
| ORF Start: at 1 | ORF Stop: end of sequence |

| | | |
|---|---|---|
| SEQ ID NO: 156 | 501 aa | MW at 56709.5 kD |

| | |
|---|---|
| NOV23v, | KFNVLEIVQKVLHKDKSLENLGMLRNGGLLFRMTLLTSGGAGMLYVRWRIMGTGPPAF |
| CG57774-03 | |
| Protein Sequence | TEVDNPASFADSMLVRAVNYNYYYSLNAWLLLCPWWLCFDWSMGCIPLIKSISDWRVI |
| | ALAALWFCLIGLICQALCSEDGHKRRILTLGLGFLVIPFLPASNLFFRVCFVVAERVL |
| | YLPSVGYCVLLTFGFGALSKHTKKKKLIAAVVLGILFINTLRCVLRSGEWRSEEQLFR |
| | SALSVCPLNAKVHYNIGKNLADKGNQTAAIRYYREAVRLNPKYVHANNNLGNILKERN |
| | ELQEAEELLSLAVQIQPDFAAAWMNLGIVQNSLKRFEAAEQSYRTAIKHRRKYPDCYY |
| | NLGRLYADLNRHVDALNAWRNATVLKPEHSLAWNNMIILLDNTGNLAQAEAVGREALE |
| | LIPNDHSLMFSLANVLGKSQKYKESEALFLKAIKANPNAASYHGNLAVLYHRWGHLDL |
| | AKKHYEISLQLDPTASGTKENYGLLRRKLELMQKKAV |

| | |
|---|---|
| SEQ ID NO: 157 | 1515 bp |

| | |
|---|---|
| NOV23w, | GAATTCAAATTCAATGTTCTGGAAATTCTCCAGAAGGTACTACATAAGGACAAGTCAT |
| CG57774-04 DNA | |
| Sequence | TAGAGAATCTCGGCATGCTCAGGAACGGGGGCCTCCTCTTCAGAATGACCCTGCTCAC |
| | CTCTCGAGGGGCTGGGATGCTCTACGTGCGCTGGAGGATCATGGGCACGGGCCCGCCG |
| | GCCTTCACCGAGGTGGACAACCCGGCCTCCTTTGCTGACAGCATGCTGGTGAGGGCCG |
| | TAAACTACAATTACTACTATTCATTGAATGCCTGGCTGCTGCTGTGTCCCTGGTGGCT |
| | GTGTTTTGATTGGTCAATGGGCTGCATCCCCCTCATTAAGTCCATCAGCGACTGGAGG |
| | GTAATTGCACTTGCAGCACTCTGGTTCTGCCTAATTGGCCTGATATGCCAAGCCCTGT |
| | GCTCTGAAGACGGCCACAAGAGAAGGATCCTTACTCTGGGCCTGGGATTTCTCGTTAT |
| | CCCATTTCTCCCTGCGAGTAACCTGTTCTTCCGAGTGGGCTTCGTGGTCGCGGAGCGT |
| | GTCCTCTACCTCCCCAGCGTTGGGTACTGTGTGCTGCTGACTTTTGGATTCGGAGCCC |
| | TGAGCAAACATACCAAGAAAAAGAAACTCATTGCCGCTGTCGTGCTGGGAATCTTATT |
| | CATCAACACGCTGAGATGTGTGCTGCGCAGCGGCGAGTGGCGGAGTGAGGAACAGCTT |
| | TTCAGAAGTGCTCTGTCTGTGTGTCCCCTCAATGCTAAGGTTCACTACAACATTGGCA |
| | AAAACCTGGCTGATAAAGGCAACCAGACACCTGCCATCAGATACTACCGGGAAGCTGT |
| | AAGATTAAATCCCAAGTATGTTCATGCCATGAATAATCTTGGAAATATCTTAAAAGAA |
| | AGGAATGAGCTACAGCAAGCTGAGGAGCTGCTGTCTTTGGCTGTTCAAATACAGCCAG |
| | ACTTTGCCGCTCCGTGGATGAATCTAGGCATAGTGCAGAATAGCCTGAAACGGTTTGA |
| | AGCAGCAGAGCAAAGTTACCGGACAGCAATTAAACACAGAAGGAAATACCCAGACTGT |
| | TACTACAACCTCGGGCGTCTGTATGCAGATCTCAATCGCCACGTGGATGCCTTGAATG |
| | CGTGGAGAAATGCCACCGTGCTGAAACCAGAGCACAGCCTGGCCTGGAACAACATGAT |
| | TATACTCCTCGACAATACAGGTAATTTAGCCCAAGCTGAAGCAGTTGGAAGAGAGGCA |
| | CTGGAATTAATACCTAATGATCACTCTCTCATGTTCTCGTTGGCAAACGTGCTGGGGA |
| | AATCCCAGAAATACAAGGAATCTGAAGCTTTATTCCTCAAGGCAATTAAAGCAAATCC |
| | AAATGCTGCAAGTTACCATGGTAATTTGGCTGTGCTTTATCATCGTTGGGGACATCTA |
| | GACTTGGCCAAGAAACACTATGAAATCTCCTTGCAGCTTGACCCCACGGCATCAGGAA |
| | CTAAGGAGAATTACGGTCTGCTGAGAAGAAAGCTAGAACTAATGCAAAAGAAAGCTGT |
| | CCTCGAG |

| | |
|---|---|
| ORF Start: at 7 | ORF Stop: at 1510 |

| | | |
|---|---|---|
| SEQ ID NO: 158 | 501 aa | MW at 56709.5 kD |

| | |
|---|---|
| NOV23w, | KFNVLEIVQKVLHKDKSLENLGMLRNGGLLFRMTLLTSGGAGMLYVRWRIMGTGPPAF |
| CG57774-04 | |
| Protein Sequence | TEVDNPASFADSMLVRAVNYNYYYSLNAWLLLCPWWLCFDWSMGCIPLIKSISDWRVI |
| | ALAALWFCLIGLICQALCSEDGHKRRILTLGLGFLVIPFLPASNLFFRVGFVVAERVL |
| | YLPSVGYCVLLTFGFGALSKHTKKKKLIAAVVLGILFINTLRCVLRSGEWRSEEQLFR |
| | SALSVCPLNAKVHYNIGKNLADKGNQTAAIRYYREAVRLNPKYVHAMNNLGNILKERN |
| | ELQEAEELLSLAVQIQPDFAAAWMNLGIVQNSLKRFEAAEQSYRTAIKHRRKYPDCYY |
| | NLGRLYADLNPHVDALNAWRNATVLKPEHSLAWNNMIILLDNTGNLAQAEAVGREAIE |
| | LIPNDHSLMFSLANVLGKSQKYKESEALFLKAIKANPNAASYHCNLAVLYHRWGHLDL |
| | AKKHYEISLQLDPTASGTKENYGLLRRKLELMQKKAV |

| | |
|---|---|
| SEQ ID NO: 159 | 1515 bp |

| | |
|---|---|
| NOV23x, | GAATTCAAATTCAATGTTCTGGAAATTGTCCAGAAGGTACTACATAAGGACAAGTCAT |
| CG57774-05 DNA | |
| Sequence | TAGAGAATCTCGGCATGCTCAGGAACGGGGGCCTCCTCTTCAGAATGACCCTGCTCAC |
| | CTCTGGAGGGGCTCGGATGCTCTACGTGCGCTGGAGGATCATGGGCACGGGCCCGCCG |
| | GCCTTCACCGAGGTGGACAACCCGGCCTCCTTTGCTGACAGCATGCTGGTGAGGGCCG |
| | TAAACTACAATTACTACTATTCATTGAATGCCTGGCTGCTGCTGTGTCCCTGGTGGCT |
| | GTGTTTTCATTGGTCAATGGGCTGCACCCCCCTCATTAAGTCCATCAGCGACTGGAGG |
| | GTAATTGCACTTGCAGCACTCTGGTTCTGCCTAATTGGCCTGATATGCCAAGCCCTGT |
| | GCTCTGAAGACGGCCACAAGAGAAGGATCCTTACTCTGGGCCTGGGATTTCTCGTTAT |
| | CCCATTTCTCCCTGCGAGTAACCTGTTCTTCCGAGTGGGCTTCGTGGTCGCGGAGCGT |
| | GTCCTCTACCTCCCCAGCGTTGGGTACTGTGTGCTGCTGACTTTTCGATTCGGAGCCC |
| | TGAGCAAACATACCAAGAAAAAGAAACTCATTGCCGCTGTCGTGCTGGGAATCTTATT |
| | CATCAACACGCTGAGATGTGTGCTGCGCAGCGGCGAGTGGCGGAGTGAGGAACAGCTT |
| | TTCAGAAGTGCTCTGTCTGTGTGTCCCCTCAATGCTAAGGTTCACTACAACATTGGCA |
| | AAAACCTGGCTGATAAAGGCAACCAGACAGCTGCCATCAGATACTACCGGGAAGCTGT |
| | AAGATTAAATCCCAAGTATGTTCATGCCATGAATAATCTTGGAAATATCTTAAAAGAA |
| | AGGAATGAGCTACAGGAAGCTGAGGAGCTGCTGTCTTTGGCTGTTCAAATACAGCCAG |
| | ACTTTGCCGCTGCGTGGATGAATCTAGGCATAGTGCAGAATAGCCTGAAACGGTTTGA |
| | AGCAGCAGAGCAAAGTTACCGGACAGCAATTAAACACAGAAGGAAATACCCAGACTGT |
| | TACTACAACCTCGGGCGTCTGTATGCAGATCTCAATCGCCACGTGGATGCCTTGAATG |
| | CGTGGAGAAATGCCACCGTCCTGAAACCAGAGCACAGCCTGGCCTGGAACAACATGAT |
| | TATACTCCTCGACAATACAGGTAATTTAGCCCAAGCTGAAGCAGTTGGAAGAGAGGCA |
| | CTGGAATTAATACCTAATGATCACTCTCTCATGTTCTCGTTGGCAAACGTGCTGGGGA |
| | AATCCCAGAAATACAAGGAATCTGAAGCTTTATTCCTCAAGGCAATTAAAGCAAATCC |
| | AAATGCTGCAAGTTACCATGGTAATTTGGCTGTGCTTTATCATCGTTGGGGGCATCTA |
| | GACTTGGCCAAGAAACACTATGAAATCTCCTTGCAGCTTGACCCCACGGCATCAGGAA |
| | CTAAGGAGAATTACGGTCTGCTGAGAAGAAAGCTAGAACTAATGCAAAAGAAAGCTGT |
| | CCTCGAG |

| | |
|---|---|
| ORF Start: at 7 | ORF Stop: at 1510 |

| | | |
|---|---|---|
| SEQ ID NO: 160 | 501 aa | MW at 56697.5 kD |

| | |
|---|---|
| NOV23x, | KFNVLEIVQKVLHKDKSLENLGMLRNGGLLFRMTLLTSGGAGMLYVRWRIMGTGPPAF |
| CG57774-05 | |
| Protein Sequence | TEVDNPASFADSMLVRAVNYNYYYSLNAWLLLCPWWLCFDWSMGCTPLIKSISDWRVI |
| | ALAALWFCLIGLICQALCSEDGHKRRILTLGLGFLVIPFLPASNLFFRVGFVVAERVL |
| | YLPSVGYCVLLTFGFGALSKHTKKKKLIAAVVLGILFINTLRCVLRSGEWRSEEQLFR |
| | SALSVCPLNAKVhYNIGKNLADKGNQTAAIRYYREAVRLNPKYVHAMNNLGNILKERN |
| | ELQEAEELLSLAVQIQPDFAAAWMNLGIVQNSLKRFEAAEQSYRTAIKHRRKYPDCYY |
| | NLGRLYADLNRHVDALNAWRNATVLKPEHSLAWNNMIILLDNTGNLAQAEAVGREALE |
| | LIPNDHSLMFSLANVLGKSQKYKESEALFLKAIKANPNAASYHGNLAVLYHRWGHLDL |
| | AKKHYEISLQLDPTASGTKENYGLLRRKLELMQKKAV |

| | |
|---|---|
| SEQ ID NO: 161 | 855 bp |

| | |
|---|---|
| NOV23y, | GAATTCAGCGGCGAGTGGCGGAGTGAGGAACAGCTTTTCAGAAGTGCTCTGTCTGTGT |
| CG57774-06 DNA | |
| Sequence | GTCCCCTCAATGCTAAGGTTCACTACAACATTGGCAAAAACCTGGCTGATAAAGGCAA |
| | CCAGACAGCTGCCATCAGATACTACCGGGAAGCTGTAAGATTAAATCCCAAGTATGTT |
| | CATGCCATGAATAATCTTGGAAATATCTTAAAAGAAAGGAATGAGCTACAGGAAGCTG |
| | AGGAGCTGCTGTCTTTGGCTGTTCAAATACAGCCAGACTTTGCCGCTGCGTGGATGAA |
| | TCTAGGCATAGTGCAGAATAGCCTGAAACGGTTTGAAGCAGCAGAGCAAAGTTACCGG |
| | ACAGCAATTAAACACAGAAGGAAATACCCAGACTGTTACTACAACCTCGGGCGTCTGT |
| | ATGCAGATCTCAATCGCCACGTGGATGCCTTGAATGCGTGGAGAAATGCCACCGTGCT |
| | GAAACCAGAGCACAGCCTGGCCTGGAACAACATGATTATACTCCTCGACAATACAGGT |
| | AATTTAGCCCAAGCTGAAGCAGTTGGAAGAGAGGCACTGGAATTAATACCTAATGATC |
| | ACTCTCTCATGTTCTCGTTGGCAAACGTGCTGGGGAAATCCCAGAAATACAAGGAATC |
| | TGAAGCTTTATTCCTCAAGGCAATTAAAGCAAATCCAAATGCTGCAAGTTACCATGGT |
| | AATTTGGCTGTGCTTTATCATCGTTGGGGACATCTAGACTTGGCCAAGAAACACTATG |
| | AAATCTCCTTGCAGCTTGACCCCACGGCATCAGGAACTAAGGAGAATTACGGTCTGCT |
| | GAGAAGAAAGCTAGAACTAATGCAAAAGAAAGCTGTCCTCCAG |

| | |
|---|---|
| ORF Start: at 7 | ORF Stop: at 850 |

| | | |
|---|---|---|
| SEQ ID NO: 162 | 281 aa | MW at 31997.2 kD |

| | |
|---|---|
| NOV23y, | SGEWRSEEQLFRSALSVCPLNAKVHYNIGKNLADKGNQTAAIRYYREAVRLNPKYVHA |
| CG57774-06 | |
| Protein Sequence | MNNLGNILKERNELQEAEELLSLAVQIQPDFAAAWMNLGIVQNSLKRFEAAEQSYRTA |
| | IKHRRKYPDCYYNLGRLYADLNRHVDALNAWRNATVLKPEHSLAWNNMIILLDNTGNL |
| | AQAEAVGREALELIPNDHSLMFSLANVLGKSQKYKESEALFLKAIKANPNAASYHGNL |
| | AVLYHRWGHLDLAKKHYEISLQLDPTASGTKENYGLLRRKLELMQKKAV |

| | |
|---|---|
| SEQ ID NO: 163 | 855 bp |

| | |
|---|---|
| NOV23z, | GAATTCAGCGGCGAGTGGCGGAGTGAGGAACAGCTTTTCAGAAGTGCTCTGTCTGTGT |
| CG57774-07 DNA | |
| Sequence | GTCCCCTCAATGCTAAGGTTCACTACAACATTGGCAAAAACCTGGCTGATAAAGGCAG |
| | CCAGACAGCTGCCATCAGATACTACCGGGAAGCTGTAAGATTAAATCCCAAGTATGTT |
| | CATGCCATGAATAATCTTGGAAATATCTTAAAAGAAAGGAATGAGCTACAGGAAGCTG |
| | AGGAGCTGCTGTCTTTGGCTGTTCAAATACAGCCAGACTTTGCCGCTGCGTGGATGAA |
| | TCTAGGCATAGTGCAGAATAGCCTGAAACGGTTTGAAGCAGCAGAGCAAAGTTATCGG |
| | ACAGCAATTAAACACAGAADGAAATACCCAGACTGTTACTACAACCTCGGGCGTCTGT |
| | ATGCAGATCTCAATCGCCACGTGGATGCCTTGAATGCGTGGAGAAATGCCACCGTGCT |
| | CAAACCAGAGCACAGCCTGGCCTGGAACAACATGATTATACTCCTCGACAATACAGGT |
| | AATTTAGCCCAAGCTGAAGCAGTTGGAAGAGAGGCACTGGAATTAATACCTAATGATC |
| | ACTCTCTCATGTTCTCGTTGGCAAACGTGCTGGGGAAATCCCAGAAATACAAGGAATC |
| | TGAAGCTTTATTCCTCAAGGCAATTAAAGCAAATCCAAATGCTGCAAGTTACCATGGT |
| | AATTTCGCTGTCCTTTATCATCGTTGGGGACATCTAGACTTGGCCAAGAAACACTATG |
| | AAATCTCCTTGCAGCTTGACCCCACGGCATCAGGAACTAAGGAGAATTACGGTCTGCT |
| | GAGAAGAAAGCTAGAACTAATGCAAAAGAAAGCTGTCCTCGAG |

| | |
|---|---|
| ORF Start: at 7 | ORF Stop: at 850 |

| | | |
|---|---|---|
| SEQ ID NO: 164 | 281 aa | MW at 31970.1 kD |

| | |
|---|---|
| NOV23z, | SGEWRSEEQLFRSALSVCPLNAKVHYNIGKNLADKGSQTAAIRYYREAVRLNPKYVHA |
| CG57774-07 | |
| Protein Sequence | MNNLGNILKERNELQEAEELLSLAVQIQPDFAAAWMNLGIVQNSLKRFEAAEQSYRTA |
| | IKHRRKYPDCYYNLGRLYADLNRHVDALNAWRNATVLKPEHSLAWNNMIILLDNTGNL |
| | AQAEAVGREALELIPNDHSLMFSLANVLGKSQKYKESEALFLKAIKANPNAASYHGNL |
| | AVLYHRWGHLDLAKKHYEISLQLDPTASGTKENYGLLRRKLELMQKKAV |

| | |
|---|---|
| SEQ ID NO: 165 | 855 bp |

| | |
|---|---|
| NOV23aa, | GAATTCAGCGGCGAGTGGCGGAGTGAGGAACAGCTTTTCAGAAGTGCTCTGTCTGTGT |
| CG57774-08 DNA | |
| Sequence | GTCCCCTCAATGCTAAGGTTCACCACAACATTGGCAAAAACCTGGCTGATAAAGGCAA |
| | CCAGACAGCTGCCATCAGATACTACCGGGAAGCTGTAAGATTAAATCCCAAGTATGTT |
| | CATGCCATGAATAATCTTGGAAATATCTTAAAAGAAAGGAATGAGCTACAGGAAGCTG |
| | AGGACCTGCTGTCTTTGGCTGTTCAAATACAGCCAGACTTTGCCGCTGCGTGGATGAA |
| | TCTAGGCATAGTGCAGAATAGCCTGAAACGGTTTGCCGCAGCAGAGCAAAGTTACCCG |
| | ACAGCAATTAAACACAGAAGGAAATACCCAGACTGTTACTACAACCTCGGGCGTCTGT |
| | ATGCAGATCTCAATCGCCACGTGGATGCCTTGAATGCGTGGAGAAATGCCACCCTGCT |
| | GAAACCAGAGCACAGCCTGGCCTCGAACAACATGATTATACTCCTCGACAATACAGGT |
| | AATTTAGCCCAAGCTGAAGCAGTTGGAAGAGAGGCACTGGAATTAATACCTAATGATC |
| | ACTCTCTCATGTTCTCGTTGGCAACGTGCTGGGGAAAATCCCAGAAATACAAGGAATC |
| | TGAAGCTTTATTCCTCAAGGCAATTAAAGCAAATCCAAATGCTGCAAGTTACCATGGT |
| | AATTTGGCTGTGCTTTATCATCGTTGGGGACATCTAGACTTGGCCAAGAAACACTATG |
| | AAATCTCCTTGCAGCTTGACCCCACGGCATCAGGAACTAAGGAGAATTACGGTCTGCT |
| | GAGAAGAAAGCTAGAACTAATGCAAAAGAAAGCTGTCCTCGAG |

| | |
|---|---|
| ORF Start: at 7 | ORF Stop: at 850 |

| | | |
|---|---|---|
| SEQ ID NO: 166 | 281 aa | MW at 31971.1 kD |

| | |
|---|---|
| NOV23aa, | SGEWRSEEQLFRSALSVCPLNAKVHHNIGKNLADKGNQTAAIRYYREAVRLNPKYVHA |
| CG57774-08 | |
| Protein Sequence | MNNLGNILKERNELQEAEELLSLAVQIQPDFAAAWMNLGIVQNSLKRFEAAEQSYRTA |
| | IKHRRKYPDCYYNLGRLYADLNRHVDALNAWRNATVLKPEHSLAWNNMIILLDNTGNL |
| | AQAEAVGREALELIPNDHSLMFSLANVLGKSQKYKESEALFLKAIKANPNAASYHGNL |
| | AVLYHRWGHLDLAKKHYEISLQLDPTASGTKENYGLLRRKLELMQKKAV |

| | |
|---|---|
| SEQ ID NO: 167 | 855 bp |

| | |
|---|---|
| NOV2ab, | GAATTCAGCGGCGAGTGGCGGAGTGAGGAACAGCTTTTCAGAAGTGCTCTGTCTGTGT |
| CG57774-09 DNA | |
| Sequence | GTCCCCTCAATGCTAAGGTTCACTACAACATTGGCAAAAACCTGGCTGATAAAGGCAA |
| | CCAGACAGCCGCCATCAGATACTACCGGGAAGCTGTAAGATTAAATCCCAAGTATGTT |
| | CATGCCATGAATAATCTTGGAAATATCTTAAAAGAAAGGAATGAGCTACAGGAAGCTG |
| | AGGAGCTGCTGTCTTTGGCTGTTCAAATACAGCCAGACTTTGCCGCTGCGTGGATGAA |
| | TCTAGGCATAGTGCAGAATAGCCTGAAACGGTTTGAAGCAGCAGAGCAAAGTTACCGG |
| | ACAGCAATTAAACACAGAAGGAAATACCCAGACTGTTACTACAACCTCGGGCGTCTGT |
| | ATGCAGATCTCAATCGCCACGTGGATGCCTTGAATGCGTGGAGAAATCCCACCGTGCT |
| | GAAACCAGAGCACAGCCTGGCCTGGAACAACATGATTATACTCCTCGACAATACAGGT |
| | AATTTAGCCCAAGCTGAAGCAGTTGGAAGAGAGGCACTGGAATTAATACCTAATGATC |
| | ACTCTCTCATGTTCTCGTTGGCAAACGTGCTGGGGAAATCCCAGAAATACAAGGAATC |
| | TGAAGCTTTATTCCTCAAGGCAATTAAAGCAAATCCAAATGCTGCAAGTTACCATGGT |
| | AATTTGGCTGTGCTTTATCATCGTTGGGGACATCTAGACTTGGCCAAGAAACACTATG |
| | AAATCTCCTTGCAGCTTCACCCCACGGCATCAGGAACTAAGGAGAATTACCGTCTGCT |
| | GAGAAGAAAGCTAGAACTAATGCAAAAGAAAGCTGTCCTCGAG |

| | |
|---|---|
| ORF Start: at 7 | ORF Stop: at 850 |

| | | |
|---|---|---|
| SEQ ID NO: 168 | 281 aa | MW at 31997.2 kD |

| | |
|---|---|
| NOV23ab, | SGEWRSEEQLFRSALSVCPLNAKVHYNIGKNLADKGNQTAAIRYYREAVRLNPKYVHA |
| CG57774-09 | |
| Protein Sequence | MNNLGNILKERNELQEAEELLSLAVQIQPDFAAAWMNLGIVQNSLKRFEAAEQSYRTA |
| | IKHRRKYPDCYYNLGRLYADLNRHVDALNAWRNATVLKPEHSLAWNNMIILLDNTGNL |
| | AQAEAVGREALELIPNDHSLMFSLANVLGKSQKYKESEALFLKAIKANPNAASYHGNL |
| | AVLYHRWGHLDLAKKNYEISLQLDPTASGTKENYGLLRRKLELMQKKAV |

| | |
|---|---|
| SEQ ID NO: 169 | 855 bp |

| | |
|---|---|
| NOV23ac, | GAATTCAGCGGCGAGTGGCGGAGTGAGGAACAGCTTTTCAGAAGTGCTCTGTCTGTGT |
| CG57774-10 DNA | |
| Sequence | GTCCCCTCAATGCTAAGGTTCACTACAACATTGGCAAAAACCTGGCTGATAAAGGCAA |
| | CCAGACAGCTGCCATCAGATACTACCGGGAAGCTGTAAGATTAAATCCCAAGTATGTT |
| | CATGCCATGAATAATCTTGGAAATATCTTAAAAGAAAGGAATGAGCTACAGGAAGTTG |
| | AGGAGCTGCTGTCTTTGGCTGTTCAAATACAGCCAGACTTTGCCGCTGCGTGGATGAA |
| | TCTAGGCATAGTGCAGAATAGCCTGAAACGGTTTGAAGCAGCAGAGCAAAGTTACCGG |
| | ACAGCAATTAAACACAGAAGGAAATACCCAGACTCTTACTACAACCTCGGGCGTCTGT |
| | ATGCAGATCTCAATCGCCACGTGGATGCCTTGAATGCGTGGAGAAATGCCACCGTGCT |
| | GAAACCAGAGCACAGCCTGGCCTGGAACAACATGATTATACTCCTCGACAATACAGGT |
| | AATTTAGCCCAAGCTGAAGCAGTTGGAAGAGAGGCACTGGAATTAATACCTAATGATC |
| | ACTCTCTCATGTTCTCGTTGGCAAACGTGCTGGGGAAATCCCAGAAATACAAGGAATC |
| | TGAAGCTTTATCCCTCAAGGCAATTAAAGCAAATCCAAATGCTGCAAGTTACCATGGT |
| | AATTTGGCTGTGCTTTATCATCGTTGGGGACATCTAGACTTGGCCAAGAAACACTATG |
| | AAATCTCCTTGCAGCTTGACCCCACGGCATCAGGAACTAAGGAGAATTACGGTCTGCT |
| | GAGAAGAAAGCTAGAACTAATGCAAAAGAAAGCTGTCCTCGAG |

| | |
|---|---|
| ORF Start: at 7 | ORF Stop: at 850 |

| | | |
|---|---|---|
| SEQ ID NO: 170 | 281 aa | MW at 32025.2 kD |

| | |
|---|---|
| NOV23ac, | SGEWRSEEQLFRSALSVCPLNAKVHYNIGKNLADKGNQTAAIRYYREAVRLNPKYVHA |
| CG57774-10 | |
| Protein Sequence | MNNLGNILKERNELQEVEELLSLAVQIQPDFAAAWMNLGIVQNSLKRFEAAEQSYRTA |
| | IKHRRKYPDCYYNLGRLYADLNRHVDALNAWRNATVLKPEHSLAWNNMIILLDNTGNL |
| | AQAEAVGREALELIPNDHSLMFSLANVLGKSQKYKESEALFLKAIKANPNAASYHGNL |
| | AVLYHRWGHLDLAKKHYEISLQLDPTASGTKENYGLLRRKLELMQKKAV |

| | |
|---|---|
| SEQ ID NO: 171 | 855 bp |

| | |
|---|---|
| NOV23ad, | GAATTCAGCGGCGAGTGGCGGAGTGAGGAACAGCTTTTCAGAAGTGCTCTGTCTGTGT |
| CG57774-11 DNA | |
| Sequence | GTCCCCTCAATGCTAAGGTTCACTACAACATTGGCAAAAACCTCGCTGATAAAGGCAA |
| | CCAGACAGCTGCCATCAGATACTACCGGGAAGCTGTAAGATTAAATCCCAAGTATGTT |
| | CATGCCATGAATAATCTTGGAAATATCTTAAAAGAAAGGAATGAGCTACAGGAAGCTG |
| | AGGAGCTGCTGTCTTTGGCTGTTCAAATACAGCCAGACTTTGCCGCTGCGTGGATGAA |
| | TCTAGGCATAGTGCAGAATAGCCTGAAACGGTTTGAAGCAGCAGAGCAAAGTTACCCG |
| | ACAGCAATTAAACACAGAAGGAAATACCCAGACTGTTACTACAACCTCGGGCGTCTGT |
| | ATGCAGATCTCAATCCCCACGTGGATGCCTTGAATGCGTGGAGAAATGCCACCGTGCT |
| | GAAACCAGAGCACAGCCTGGCCTGGAACAACATGATTATACTCCTCGACAATACAGGT |
| | AATTTAGCCCAAGCTGAAGCAGTTGGAAGAGAGGCACTGGAATTAATACCTAATGATC |
| | ACTCTCTCATGTTCTCGTTGGCAAACGTGCTGGGGAAATCCCAGAAATACAAGGAATC |
| | TGAAGCTTTATCCCTCAAGGCAATTAAAGCAAATCCAAATGCTGCAAGTTACCATGGT |
| | AATTTGGCTGTGCTTTATCATCGTTGGGGACATCTAGACTTGGCCAAGAAACACTATG |
| | AAATCTCCTTCCAGCTTGACCCCACGGCATCAGGAACTAAGGAGAATTACGGTCTGCT |
| | GAGAAGAAAGCTAGAACTAATGCAAAAGAAAGCTGTCCTCGAG |

| | |
|---|---|
| ORF Start: at 7 | ORF Stop: at 850 |

| | | |
|---|---|---|
| SEQ ID NO: 172 | 281 aa | MW at 31937.1 kD |

| | |
|---|---|
| NOV23ad, | SGEWRSEEQLFRSALSVCPLNAKVHYNIGKNLADKGNQTAAIRYYREAVRLNPKYVHA |
| CG57774-11 | |
| Protein Sequence | MNNLGNILKERNELQEAEELLSLAVQIQPDFAAAWMNLGIVQNSLKRFEAAEQSYRTA |
| | IKHRRKYPDCYYNLGRLYADLNRHVDALNAWRNATVLKPEHSLAWNNMIILLDNTGNL |
| | AQAEAVGREALELIPNDHSLMFSLANVLGKSQKYKESEALSLKAIKANPNAASYHGNL |
| | AVLYHRWGHLDLAKKHYEISLQLDPTASGTKENYGLLRRKLELMQKKAV |

| | |
|---|---|
| SEQ ID NO: 173 | 855 bp |

| | |
|---|---|
| NOV23ae, | GAATTCAGCGGCGAGTGGCGGAGTGAGGAACAGCTTTTCAGAAGTGCTCTGTCTGTGT |
| CG57774-12 DNA | |
| Sequence | GTCCCCTCAATGCTAAGGTTCACTACAACATTGGCAAAAACCTGGCTGATAAAGGCAA |
| | CCAGACAGCTGCCATCAGATACTACCGGGAAGCTGTAAGATTAAATCCCAAGTATGTT |
| | CATGCCATGAATAATCTTGGAAATATCTTAAAAGAAAGGAATGAGCTACAGGAAGCTG |
| | AGGAGCTGCTGTCTTTGGCTGTTCAAATACAGCCAGACTTTGCCGCTGCGTGGATGAA |
| | TCTAGGCATAGTGCAGAATAGCCTGAAACGGTTTGAAGCAGCAGAGCAAAGTTACCGG |
| | ACAGCAATTAAACACAGAAGGAAATACCCAGACTGTTACTACAACCTCGGGCGTCTGT |
| | ATGCAGATCTCAATCGCCACGTGGATGCCTTGAATGCGTGGAGAAATGCCACCGTGCT |
| | GAAACCACAGCACAGCCTGGCCTGGAACAACATGATTATACTCCTCGACAATACAGGT |
| | AATTTAGCCCAAGCTGAAGCAGTTGGAAGAGAGGCACTGGAATTAATACCTAATGATC |
| | ACTCTCTCATGTTCTCGTTGGCAAACGTGCTGGGGAAATCCCAGAAATACAAGGAATC |
| | TCAAGCTTTATTCCTCAAGGCAATTAAAGCAAATCCAAATGCTGCAAGTTACCATGGT |
| | AATTTGGCTGTGCTTTATCATCGTTGGGGACATCTAGACTTGGCCAAGAAACACCATG |
| | AAATCTCCTTGCAGCTTGACCCCACGGCATCAGGAACTAAGGAGAATTACGGTCTCCT |
| | GAGAAGAAAGCTAGAACTAATGCAAAACAAAGCTGTCCTCGAG |

| | |
|---|---|
| Start: at 7 | ORF Stop: at 850 |

| | | |
|---|---|---|
| SEQ ID NO: 174 | 281 aa | MW at 31971.1 kD |

| | |
|---|---|
| NOV23ae, | SGEWRSEEQLFRSALSVCPLNAKVHYNIGKNLADKGNQTAAIRYYREAVRLNPKYVHA |
| CG57774-12 | |
| Protein Sequence | MNNLGNILKERNELQEAEELLSLAVQIQPDFAAAWMNLGIVQNSLKRFEAAEQSYRTA |
| | IKHRRKYPDCYYNLGRLYADLNRHVDALNAWRNATVLKPEHSLAWNNMIILLDNTGNL |
| | AQAEAVCREALELIPNDHSLMFSLANVLGKSQKYKESEALFLKAIKANPNAASYHGNL |
| | AVLYHRWGHLDLAKKHHEISLQLDPTASGTKENYGLLRRKLELMQKKAV |

| | |
|---|---|
| SEQ ID NO: 175 | 855 bp |

| | |
|---|---|
| NOV23af, | GAATTCAGCGGCGAGTGGCGGAGTGAGGAACAGCTTTTCAGAAGTGCTCTGTCTGTGT |
| CG57774-13 DNA | |
| Sequence | GTCCCCTCAATGCTAAGGTTCACTACAACATTGGCAAAAACCTGGCTGATAAAGGCAA |
| | CCAGACAGCTGCCATCAGATACTACCGGGAAGCTGTAAGATTAAATCCCAAGTATGTT |
| | CATGCCATGAATAATCTTGGAAATATCTTAAAAGAAAGGAATGAGCTACAGGAAGCTG |
| | AGGAGCTGCTGTCTTTGGCTGTTCAAATACAGCCAGACTTTCCCGCTGCGTGGATGAA |
| | TCTAGGCATAGTGCAGAATAUCCTGAAACGGTTTGAAGCAGCAGAGCAAAGTTACCGG |
| | ACAGCAATTAAACACAGAACGAAATACCCAGACTGTTACTACAACCTCGGGCGTCTGT |
| | ATGCAGATCTCAATCGCCACGTGGATGCCTTGAATGCGTGGAGAAATGCCACCGTGCT |
| | GAAACCAGAGCACAGCCTGCCCTGGAACAACATGATTATACTCCTCGACAATACAGGT |
| | AATTTAGCCCAAGCTGAAGCAGTTGGAACACAGGCACTGGAATTAATACCTAATGATC |
| | ACTCTCTCATGTTCTCGTTGGCAAACGTGCTGGGGAAATCCCAGAAATACAGGGAATC |
| | TGAAGCTTTATTCCTCAAGGCAATTAAAGCAAATCCAAATGCTGCAAGTTACCATGGT |
| | AATTTGGCTGTGCTTTATCATCGTTGGGGACATCTAGACTTGGCCAAGAAACACTATG |
| | AAATCTCCTTGCAGCTTGACCCCACGGCATCAGGAACTAAGGAGAATTACGGTCTGCT |
| | GAGAAGAAAGCTAGAACTAATGCAAAAGAAAGCTGTCCTCGAG |

| | |
|---|---|
| ORF Start: at 7 | ORF Stop: at 850 |

| | | |
|---|---|---|
| SEQ ID NO: 176 | 281 aa | MW at 32025.2 kD |

| | |
|---|---|
| NOV23af, | SGEWRSEEQLFRSALSVCPLNAKVHYNIGKNLADKGNQTAAIRYYREAVRLNPKYVHA |
| CG57774-13 | |
| Protein Sequence | MNNLGNILKERNELQEAEELLSLAVQIQPDFAAAWMNLGIVQNSLKRFEAAEQSYRTA |
| | IKERRKYPDCYYNLGRLYADLNRHVDALNAWRNATVLKPEHSLAWNNMIILLDNTGNL |
| | AQAEAVGREALELIPNDHSLMFSLANVLGKSQKYRESEALFLKAIKANPNAASYHGNL |
| | AVLYHRWGIILDLAKKHYEISLQLDPTASGTKENYGLLRRKLELMQKKAV |

Sequence comparison of the above protein sequences yields the following sequence relationships shown in Table 23B.

TABLE 23B
Comparison of NOV23a against NOV23b through NOV23af.

| | | Identities/ |
|---|---|---|
| | | Similarities for |
| Protein | NOV23a Residues/ | the Matched |
| Sequence | Match Residues | Region |
| NOV23b | 24 . . . 524 | 501/501 (100%) |
| | 3 . . . 503 | 501/501 (100%) |
| NOV23c | 24 . . . 524 | 500/501 (99%) |
| | 3 . . . 503 | 500/501 (99%) |
| NOV23d | 24 . . . 524 | 500/501 (99%) |
| | 3 . . . 503 | 500/501 (99%) |
| NOV23e | 24 . . . 524 | 499/501 (99%) |
| | 3 . . . 503 | 499/501 (99%) |
| NOV23f | 24 . . . 524 | 500/501 (99%) |
| | 3 . . . 503 | 500/501 (99%) |
| NOV23g | 24 . . . 524 | 501/501 (100%) |
| | 3 . . . 503 | 501/501 (100%) |
| NOV23h | 24 . . . 524 | 500/501 (99%) |
| | 3 . . . 503 | 500/501 (99%) |
| NOV23i | 24 . . . 524 | 499/501 (99%) |
| | 3 . . . 503 | 499/501 (99%) |
| NOV23j | 24 . . . 524 | 500/501 (99%) |
| | 3 . . . 503 | 501/501 (99%) |
| NOV23k | 24 . . . 524 | 500/501 (99%) |
| | 3 . . . 503 | 501/501 (99%) |
| NOV23l | 24 . . . 524 | 499/501 (99%) |
| | 3 . . . 503 | 500/501 (99%) |
| NOV23m | 244 . . . 524 | 280/281 (99%) |
| | 3 . . . 283 | 281/281 (99%) |
| NOV23n | 244 . . . 524 | 280/281 (99%) |
| | 3 . . . 283 | 281/281 (99%) |
| NOV23o | 244 . . . 524 | 281/281 (100%) |
| | 3 . . . 283 | 281/281 (100%) |
| NOV23p | 244 . . . 524 | 280/281 (99%) |
| | 3 . . . 283 | 280/281 (99%) |
| NOV23q | 244 . . . 524 | 280/281 (99%) |
| | 3 . . . 283 | 280/281 (99%) |
| NOV23r | 244 . . . 524 | 280/281 (99%) |
| | 3 . . . 283 | 281/281 (99%) |
| NOV23s | 244 . . . 524 | 280/281 (99%) |
| | 3 . . . 283 | 281/281 (99%) |
| NOV23t | 24 . . . 524 | 499/501 (99%) |
| | 3 . . . 503 | 500/501 (99%) |
| NOV23u | 244 . . . 524 | 281/281 (100%) |
| | 1 . . . 281 | 281/281 (100%) |
| NOV23v | 24 . . . 524 | 501/501 (100%) |
| | 1 . . . 501 | 501/501 (100%) |
| NOV23w | 24 . . . 524 | 501/501 (100%) |
| | 1 . . . 501 | 501/501 (100%) |
| NOV23x | 24 . . . 524 | 500/501 (99%) |
| | 1 . . . 501 | 500/501 (99%) |
| NOV23y | 244 . . . 524 | 281/281 (100%) |
| | 1 . . . 281 | 281/281 (100%) |
| NOV23z | 244 . . . 524 | 280/281 (99%) |
| | 1 . . . 281 | 281/281 (99%) |
| NOV23aa | 244 . . . 524 | 280/281 (99%) |
| | 1 . . . 281 | 281/281 (99%) |
| NOV23ab | 244 . . . 524 | 281/281 (100%) |
| | 1 . . . 281 | 281/281 (100%) |
| NOV23ac | 244 . . . 524 | 280/281 (99%) |
| | 1 . . . 281 | 280/281 (99%) |
| NOV23ad | 244 . . . 524 | 280/281 (99%) |
| | 1 . . . 281 | 280/281 (99%) |
| NOV23ae | 244 . . . 524 | 280/281 (99%) |
| | 1 . . . 281 | 281/281 (99%) |
| NOV23af | 244 . . . 524 | 280/281 (99%) |
| | 1 . . . 281 | 281/281 (99%) |

Further analysis of the NOV23a protein yielded the following properties shown in Table 23C.

TABLE 23C
Protein Sequence Properties NOV23a

| | |
|---|---|
| PSort | 0.6850 probability located in endoplasmic reticulum |
| analysis: | (membrane); 0.6400 probability located in plasma membrane; |
| | 0.4600 probability located in Golgi body; 0.1000 probability |
| | located in endoplasmic reticulum (lumen) |
| SignalP | Cleavage site between residues 24 and 25 |
| analysis: | |

A search of the NOV23a protein against the Geneseq database, a proprietary database that contains sequences published in patents and patent publication, yielded several homologous proteins shown in Table 23D.

TABLE 23D
Geneseq Results for NOV23a

| | | NOV23a | Identities/ | |
|---|---|---|---|---|
| | | Residues/ | Similarities | |
| Geneseq | Protein/Organism/Length | Match | for the Matched | Expect |
| Identifier | [Patent #, Date] | Residues | Region | Value |
| AAE22157 | Human TRNFR-19 protein - | 1 . . . 524 | 524/524 (100%) | 0.0 |
| | Homo sapiens, 760 aa. | 237 . . . 760 | 524/524 (100%) | |
| | [WO200226950-A2, 04 APR. | | | |
| | 2002] | | | |
| AAM41435 | Human polypeptide SEQ ID NO | 1 . . . 524 | 524/524 (100%) | 0.0 |
| | 6366 - Homo sapiens, 547 aa. | 24 . . . 547 | 524/524 (100%) | |
| | [WO200153312-A1, 26 JUL. | | | |
| | 2001] | | | |
| AAM39649 | Human polypeptide SEQ ID NO | 1 . . . 524 | 524/524 (100%) | 0.0 |
| | 2794 - Homo sapiens, 524 aa. | 1 . . . 524 | 524/524 (100%) | |
| | [WO200153312-A1, 26 JUL. | | | |
| | 2001] | | | |
| AAE05188 | Human drug metabolising | 1 . . . 524 | 523/524 (99%) | 0.0 |
| | enzyme (DME-19) protein - | 218 . . . 741 | 524/524 (99%) | |
| | Homo sapiens, 741 aa. | | | |
| | [WO200151638-A2, 19 JUL. | | | |
| | 2001] | | | |
| AAB12140 | Hydrophobic domain protein | 1 . . . 524 | 523/524 (99%) | 0.0 |
| | isolated from WERI-RB cells - | 126 . . . 649 | 524/524 (99%) | |
| | Homo sapiens, 649 aa. | | | |
| | [WO200029448-A2, 25 MAY | | | |
| | 2000] | | | |

In a BLAST search of public sequence datbases, the NOV23a protein was found to have homology to the proteins shown in the BLASTP data in Table 23E.

TABLE 23E
Public BLASTP Results for NOV23a

| | | NOV23a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9BGZ6 | Hypothetical 59.2 kDa protein - | 1 . . . 524 | 513/524 (97%) | 0.0 |
| | Macaca fascicularis (Crab | 1 . . . 524 | 519/524 (98%) | |
| | eating macaque) (Cynomolgus | | | |
| | monkey), 524 aa. | | | |
| AAH31368 | Hypothetical protein - Mus | 1 . . . 524 | 479/524 (91%) | 0.0 |
| | musculus (Mouse), 524 aa. | 1 . . . 524 | 496/524 (94%) | |
| Q96SU8 | CDNA FLJ14624 fis, clone | 46 . . . 524 | 476/479 (99%) | 0.0 |
| | NT2RP2000248, weakly similar to | 1 . . . 479 | 477/479 (99%) | |
| | UDP-N-acetylglucosamine-- | | | |
| | peptide N- | | | |
| | acetylglucosaminyltransferase | | | |
| | 110 kDa subunit (EC 2.4.1.-) - | | | |
| | Homo sapiens (Human), 479 aa. | | | |
| Q8WV63 | Hypothetical 44.5 kDa protein - | 1 . . . 376 | 376/376 (100%) | 0.0 |
| | Homo sapiens (Human), 395 aa. | 1 . . . 376 | 376/376 (100%) | |
| Q9CS83 | 5730419014Rik protein - Mus | 227 . . . 524 | 281/298 (94%) | e−163 |
| | musculus (Mouse), 298 aa | 1 . . . 298 | 287/298 (96%) | |
| | (fragment). | | | |

PFam analysis predicts that the NOV23a protein contains the domains shown in the Table 23F.

TABLE 23F
Domain Analysis of NOV23a

| | | Identities/ | |
|---|---|---|---|
| | | Similarities for | |
| Pfam | NOV23a | the Matched | Expect |
| Domain | Match Region | Region | Value |
| TPR | 265 . . . 298 | 11/34 (32%) | 1.1e−05 |
| | | 27/34 (79%) | |
| TPR | 299 . . . 332 | 10/34 (29%) | 0.0026 |
| | | 28/34 (82%) | |
| TPR | 333 . . . 366 | 9/34 (26%) | 4.8e−06 |
| | | 28/34 (82%) | |
| TPR | 367 . . . 400 | 11/34 (32%) | 7.4e−05 |
| | | 24/34 (71%) | |
| TPR | 435 . . . 468 | 10/34 (29%) | 0.88 |
| | | 22/34 (65%) | |
| TPR | 469 . . . 502 | 13/34 (38%) | 0.00063 |
| | | 24/34 (71%) | |

### Example 24

The NOV24 clone was analyzed, and the nucleotide and encoded polypeptide sequences are shown in Table 24A.

TABLE 24A
NOV24 Sequence Analysis

| | |
|---|---|
| SEQ ID NO: 177 | 2107 bp |

| | |
|---|---|
| NOV24a, | GCCTTTTTTTTTTTTTTTTTTTTTTTTTTTTTAAGAGACAGAATCTCGCTCTGCCACC |
| CG89285-01 DNA | |
| Sequence | CAGGCCGGAGTGCAGTGGCGCGATCACAGCTCACTGCAGCCTTGACCTCCCGGGCTCA |
| | AGTGATCTTCCCGCCTCAGATTCCGGAGCAGCTAGGACCCCAGACAGCACCACCACAC |
| | CTGGCTTCACACGCCTTGCCGTCCGCTGCTAGCTGATACCCCACGTGGCACTCACAGC |
| | GGCCGAGGCCCCGGACCACCTGGCACCTGTGCATGCAGCTGCCGTTCCTGTTGGCACA |
| | CGGGCTTCTACGGACACAATGCCTGCCGTCCTCCTGGAGCTGGAACCCGCGCCGGCAC |
| | TGGCAGCGATGCCGAGTGATTGTGAGCTGGACACAGTGGTGCTGGCAGCCGCCATTGC |
| | CCAGGGCGCAGGAGTTAATGGCCAGGCAGGTCCTGCTGTCAGGGAATTCAGCGGCCGC |
| | TGAATTCTAGCTAGAATTCAGCGGCCGCTGAATTCTAGCAGACGGCTTTGGAATCCAC |
| | CAGCTACATCCAGCTCCCTGAGGCAGAGTTGAGAATGGAGAGAATGTTACCTCTCCTG |
| | GCTCTGGGGCTCTTGGCGGCTGGGTTCTGCCCTGCTGTCCTCTGCCACCCTAACAGCC |
| | CACTTGACGAGGAGAATCTGACCCAGGAGAACCAAGACCGAGGGACACACGTGGACCT |
| | CGGATTAGCCTCCGCCAACGTGGACTTCGCTTTCAGCCTGTACAAGCAGTTAGTCCTG |
| | AAGGCCCCTGATAAGAATGTCATCTTCTCCCCACTGAGCATCTCCACCGCCTTGGCCT |
| | TCCTGTCTCTGGGGGCCCATAATACCACCCTGACAGAGATTCTCAAAGGCCTCAAGTT |
| | CAACCTCACGGAGACTTCTGAGGCAGAAATTCAACCAACCTTCCAGCACCTCCTGCGC |
| | ACTCTCAATCAGTCCAGCGATGAGCTGCAGCTGAGTATGGGAAATGCCATGTTTCTCA |
| | AAGAGCAACTCAGTCTGCTGGACAGGTTCACGGAGGATGCCAAGAGGCTGTATGGCTC |
| | CGAGGCCTTTGCCACTGACTTTCAGGACTCAGCTGCAGCTAAGAAGCTCATCAACGAC |
| | TACGTGAAGAATGGAACTAGGGGGAAAATCACAGATCTGATCAAGGACCTTGACTCGC |
| | AGACAATGATGGTCCTGGTGAATTACATCTTCTTTAAAGCCAAATGGGACATGCCCTT |
| | TGACCCCCAAGATACTCATCAGTCAAGGTTCTACTTGAGCAAGAAAAAGTGGGTAATG |
| | GTGCCCATGATGAGTTTGCATCACCTGACTATACCTTACTTCCGGGACGAGGAGCTGT |
| | CCTGCACCGTGGTGGAGCTGAAGTACACAGGCAATGCCAGCGCACTCTTCATCCTCCC |
| | TGATCAAGACAAGATGGAGGAAGTGGAAGCCATGCTGCTCCCAGAGACCCTGAAGCGG |
| | TGGAGAGACTCTCTGGAGTTCAGAGAGATAGGTGAGCTCTACCTGCCAAAGTTTTCCA |
| | TCTCGAGCGACTATAACCTCAACGACATACTTCTCCAGCTGGGCATTGAGGAAGCCTT |
| | CACCAGCAAGGCTGACCTGTCAGGGATCACAGGGGCCAGGAACCTAGCAGTCTCCCAG |
| | GTGGTCCATAAGGCTGTGCTTGATGTATTTGAGGAGGGCACAGAAGCATCTGCTGCCA |
| | CAGCAGTCAAAATCACCCTCCTTTCTGCATTAGTGGAGACAAGGACCATTGTGCGTTT |
| | CAACAGGCCCTTCCTGATGATCATTGTCCCTACAGACACCCAGAACATCTTCTTCATG |
| | AGCAAAGTCACCAATCCCAAGCAAGCCTAGAGCTTGCCATCAAGCAGTGGGGCTCTCA |
| | GTAAGGAACTTGGAATGCAAGCTGGATGCCTGGGTCTCTGGGCACAGCCTGGCCCCTG |
| | TGCACCGAGTGGCCATGGCATGTGTGGCCCTGTCTGCTTATCCTTGGAAGGTGACAGC |
| | GATTCCCTGTGTAGCTCTCACATGCACAGGGGCCCATGGACTCTTCAGTCTGGAGGGT |
| | CCTGGGCCTCCTGACAGCAATAAATAATTTCGTTGGAAGGGCGATTCCAGCACACTTG |
| | TGGGCGACAATAAGTTTAA |

| | |
|---|---|
| ORF Start: ATG at 557 | ORF Stop: TAG at 1826 |

| | | |
|---|---|---|
| SEQ ID NO: 178 | 423 aa | MW at 47664.3 kD |

| | |
|---|---|
| NOV24a, | MERMLPLLALGLLAAGFCPAVLCHPNSPLDEENLTQENQDRGTHVDLGLASAAVDFAF |
| CG89285-01 | |
| Protein Sequence | SLYKQLVLKAPDKNVIFSPLSISTALAFLSLGAHNTTLTEILKGLKFNLTETSEAEIH |
| | QTFQHLLRTLNQSSDELQLSMGNAMFVKEQLSLLDRFTEDAKRLYGSEAFATDFQDSA |
| | AAKKLINDYVKNGTRGKITDLIKDLDSQTMMVLVNYIFFKAKWEMPFDPQDTHQSRFY |
| | LSKKKWVMVPMMSLHHLTIPYFRDEELSCTVVELKYTGNASALFILPDQDKMEEVEAA |
| | LLPETLKRWRDSLEFREIGELYLPKFSISRDYNLNDILLQLGIEEAFTSKADLSGITG |
| | ARNLAVSQVVHKAVLDVFEEGTEASAATAVKITLLSAAVETRTIVRFNRPFLMIIVPT |
| | DTQNIFFMSKVTNPKQA |

| | |
|---|---|
| SEQ ID NO: 179 | 1281 bp |

| | |
|---|---|
| NOV24b, | TACTCCAGACAGACGGCTTTGGAATCCACCAGCTACATCCAGCTCCCTGAGGCAGAGT |
| CG89285-04 DNA | |
| Sequence | TGAGAATGGAGAGAATGTTACCTCTCCTGACTCTGGGGCTCTTGGCGGCTGCGTTCTG |
| | CCCTGCTGTCCTCTGCCACCCTAACAGCCCACTTGACGAGGAGAATCTGACCCAGGAG |
| | AACCAAGACCGAGGGACACACGTGGACCTCGGATTAGCCTCCGCCAACGTCGACTTCG |
| | CTTTCAGCCTGTACAAGCAGTTAGTCCTGAAGGCCCCTGATAAGAATGTCATCTTCTC |
| | CCCACTGAGCATCTCCACCGCCTTGGCCTTCCTGTCTCTGGGGGCCCATAATACCACC |
| | CTGACAGAGATTCTCAAAGGCCTCAAGTTCAACCTCACGGAGACTTCTGAGGCAGAAA |
| | TTCACCAGAGCTTCCAGCACCTCCTGCGCACCCTCAATCAGTCCAGCGATGAGCTGCA |
| | GCTGAGTATGGGAAATGCCATGTTTGTCAAAGAGCAACTCAGTCTGCTGGACAGGTTC |
| | ACGGAGGATGCCAAGAGGCTGTATGGCTCCGAGGCCTTTGCCACTGACTTTCAGGACT |
| | CAGCTGCAGCTAAGAAGCTCATCAACGACTACGTGAAGAATGGAACTAGGGGGAAAAT |
| | CACAGATCTGATCAAGGACCTTGACTCGCAGACAATGATCGTCCTGGTGAATTACATC |
| | TTCTTTAAAGCCAAATGGGAGATGCCCTTTGACCCCCAAGATACTCATCAGTCAAGGT |
| | TCTACTTGAGCAAGAAAAAGTGGGTAATGGTGCCCATGATGAGTTTGCATCACCTGAC |
| | TATACCTTACTTCCGGGACGAGGAGCTGTCCTGCACCGTGGTGGAGCTGAAGTACACA |
| | GGCAATGCCAGCGCACTCTTCATCCTCCCTGATCAACACAAGATGGAGGAAGTGGAAG |
| | CCATGCTGCTCCCAGAGACCCTGAAGCGGTGGAGAGACTCTCTGGAGTTCAGAGAGAT |
| | AGGTGAGCTCTACCTGCCAAAGTTTTCCATCTCGAGGGACTATAACCTGAACCACATA |
| | CTTCTCCAGCTGGGCATTGAGGAAGCCTTCACCAGCAAGGCTGACCTGTCAAGGACCA |
| | TTGTGCGTTTCAACAGGCCCTTCCTGATGATCATTGTCCCTACAGACACCCAGAACAT |
| | CTTCTTCATGAGCAAAGTCACCAATCCCAAGCAAGCCTAGAGCTTGCCATCAAGCAGT |
| | GGGGCTCTCAGTAAGGAACTTGGAATTCAAACTGGATTCCTGGGTCTCTGGGCACAAC |
| | CTGGC |

| | |
|---|---|
| ORF Start: ATG at 64 | ORF Stop: TAG at 1198 |

| | | |
|---|---|---|
| SEQ ID NO: 180 | 378 aa | MW at 43117.1 kD |

| | |
|---|---|
| NOV24b, | MERMLPLLTLGLLAAGFCPAVLCHPNSPLDEENLTQENQDRGTHVDLGLASAAADFAF |
| CG89285-04 | |
| Protein Sequence | SLYKQLVLKAPDKNVIFSPLSISTALAFLSLGAHNTTLTEILKGLKFNLTETSEAEIH |
| | QSFQHLLRTLNQSSDELQLSMGNAMFVKEQLSLLDRFTEDAKRLYGSEAFATDFQDSA |
| | AAKKLINDYVKNGTRGKITDLIKDLDSQTMMVLVNYIFFKAKWEMPFDPQDTHQSRFY |
| | LSKKKWVMVPMMSLHHLTIPYFRDEELSCTVVELKYTGNASALFILPDQDKMEEVEAM |
| | LLPETLKRWRDSLEFREIGELYLPKFSISRDYNLNDTLLQLGIEEAFTSKADLSRTIV |
| | RFNRPFLMIIVPTDTQNIFFMSKVTNPKQA |

| | |
|---|---|
| SEQ ID NO: 181 | 12852 bp |

| | |
|---|---|
| NOV24c, | GCTTTGGAATCCACCAGCTACATCCAGCTCCCTGAGGCAGAGTTGAGAATGGAGAGAA |
| CG89285-03 DNA | |
| Sequence | TGTTACCTCTCCTGACTCTGGGGCTCTTGGCGGCTGGGTTCTGCCCTGCTGTCCTCTG |
| | CCACCCTAACAGCCCACTTGACGAGGAGAATCTGACCCAGGAGAACCAAGACCGAGGG |
| | ACACACGTGGACCTCGGATTAGCCTCCGCCAACGTGGACTTCGCTTTCAGCCTGTACA |
| | AGCAGTTAGTCCTGAAGGCCCCTGATAAGAATGTCATCTTCTCCCCACTGAGCATCTC |
| | CACCGCCTTGGCCTTCCTGTCTCTGGGGGCCCATAATACCACCCTGACAGAGATTCTC |
| | AAAGGCCTCAAGTTCAACCTCACGGAGACTTCTGAGGCAGAAATTCACCAAACCTTCC |
| | ACCACCTCCTGCGCACCCTCAATCAGTCCAGCGATGAGCTGCAGCTGAGTATGGGAAA |
| | TGCCATGTTTGTCAAAGAGCAACTCAGTCTGCTGGACAGGTTCACGGAGGATGCCAAG |
| | AGGCTGTATGGCTCCGAGGCCTTTGCCACTGACTTTCACGACTCAGCTGCAGCTAAGA |
| | AGCTCATCAACGACTACGTGAAGAATGGAACTAGGGGGAAAATCACAGATCTGATCAA |
| | GGACCTTGACTCGCAGACAATGATGGTCCTGGTGAATTACATCTTCTTTAAAGAGAGA |
| | TAGGTGAGCTCTACCTGCCAAAGTTTTCCATCTCGAGGGACTATAACCTGAACGACAT |
| | ACTTCTCCAGCTGGGCATTGAGGAAGCCTTCACCAGCAAGGCTGACCTGTCAGGGATC |
| | ACAGGGGCCAGGAACCTAGCAGTCTCCCAGGTGGTCCATAAGGCTGTGCTTGATGTAT |
| | TTGAGGAGGGCACAGAAGCATCTGCTGCCACAGCAGTCAAAATCACCCTCCTTTCTGC |
| | ATTAGTGGAGACAAGGACCATTGTGCGTTTCAACAGGCCCTTCCTGATGATCATTGTC |
| | CCTACAGACACCCAGAACATCTTCTTCATGAGCAAAGTCACCAATCCCAAGCAAGCCT |
| | AGAGCTTCCCATCAAGCAGTGGGGCTCTCAGTAAGGAACTTGGAATGCAAGCTGGATG |
| | CCTGGGTCTCTGGGCACAGCCTGGCCCCTGTGCACCGAGTGTCCATGGCATGTATGGC |
| | CCTGTCTGCTTATCCTTGGAAGATGACAGCGAATCCCTGTGAAGCTCTCACATGCACA |
| | GGGGCCCATGGACTCTTCATTCTGGAGGGTCCTGGGCCTCCTGACAGCAACAAATAAT |
| | ATCGTT |

| | |
|---|---|
| ORF Start: ATG at 49 | ORF Stop: TAG at 697 |

| | | |
|---|---|---|
| SEQ ID NO: 182 | 216 aa | MW at 24086.2 kD |

| | |
|---|---|
| NOV24c, | MERMLPLLTLGLLAAGFCPAVLCHPNSPLDEENLTQENQDRGTHVDLGLASAAVDFAF |
| CG89285-03 | |
| Protein Sequence | SLYKQLVLKAPDKNVIFSPLSISTALAFLSLGAHNTTLTEILKGLKFNLTETSEAEIH |
| | QTFHHLLRTLNQSSDELQLSMGNAMFVKEQLSLLDRFTEDAKRLYGSEAFATDFQDSA |
| | AAKKLINDYVKNGTRGKITDLIKDLDSQTMMVLVNYIFFKER |

| | |
|---|---|
| SEQ ID NO: 183 | 667 bp |

| | |
|---|---|
| NOV24d, | CACCAAGCTTATGGAGAGAATGTTACCTCTCCTGACTCTGGGGCTCTTGGCGGCTGGG |
| 306418132 DNA | |
| Sequence | TTCTGCCCTGCTGTCCTCTGCCACCCTAACAGCCCACTTGACGAGGAGAATCTGACCC |
| | AGGAGAACCAAGACCGAGGGACACACGTGGACCTCGGATTAGCCTCCGCCAACGTGGA |
| | CTTCGCTTTCAGCCTGTACAAGCAGTTAGTCCTGAAGGCCCCTGATAAGAATGTCATC |
| | TTCTCCCCACTGAGCATCTCCACCGCCTTGGCCTTCCTGTCTCTGGGGGCCCATAATA |
| | CCACCCTGACAGAGATTCTCAAAGGCCTCAAGTTCAACCTCACGGAGACTTCTGAGGC |
| | AGAAATTCACCAGAGCTTCCAGCACCTCCTGCGCACCCTCAATCAGTCCAGCGATGAG |
| | CTGCAGCTGAGTATGGGAAATGCCATGTTTGTCAAAGAGCAACTCAGTCTGCTGGACA |
| | GGTTCACGGAGGATGCCAAGAGGCTGTATGGCTCCGAGGCCTTTGCCACTGACTTTCA |
| | GGACTCAGCTGCAGCTAAGAAGCTCATCAACGACTACGTGAAGAATGGAACTAGGGGG |
| | AAAATCACAGATCTGATCAAGGACCTTGACTCGCAGACAATGATGGTCCTGGTGAATT |
| | ACATCTTCTTTAAAGAGAGAGTCGACGGC |

| | |
|---|---|
| ORF Start: at 2 | ORF Stop: end of sequence |

| | | |
|---|---|---|
| SEQ ID NO: 184 | 222 aa | MW at 24676.9 kD |

| | |
|---|---|
| NOV24d, | TKLMERMLPLLTLGLLAAGFCPAVLCHPNSPLDEENLTQENQDRGTHAALGLASAAVD |
| 306418132 | |
| Protein Sequence | FAFSLYKQLVLKAPDKNVIFSPLSISTALAFLSLGAHNTTLTEILKGLKFNLTETSEA |
| | EIHQSFQHLLRTLNQSSDELQLSMGNAMFVKEQLSLLDRFTEDAKRLYGSEAFATDFQ |
| | DSAAAKKLINDYVKNGTRGKITDLIKDLDSQTMMVLVNYIFFKERVDG |

| | |
|---|---|
| SEQ ID NO: 1851 | 1603 bp |

| | |
|---|---|
| NOV24e, | GACGGCTTTGGAATCCACCAGCTACATCCAGCTCCCTGAGGCAGAGTTGAGAATGGAG |
| CG89285-02 DNA | |
| Sequence | AGAATGTTACCTCTCCTGGCTCTGGGGCTCTTGGCGGCTGGGTTCTGCCCTGCTGTCC |
| | TCTGCCACCCTAACAGCCCACTTGACGAGGAGAATCTGACCCAGGAGAACCAAGACCG |
| | AGGGACACACGTGGACCTCGGATTAGCCTCCGCCAACGTGGACTTCGCTTTCAGCCTG |
| | TACAAGCAGTTAGTCCTGAAGGCCCCTGATAAGAATGTCATCTTCTCCCCACTGAGCA |
| | TCTCCACCGCCTTGGCCTTCCTGTCTCTGGGGGCCCATAATACCACCCTGACAGAGAT |
| | TCTCAAAGGCCTCAAGTTCAACCTCACGGAGACTTCTGAGGCAGAAATTCACCAGAGC |
| | TTCCAGCACCTCCTGCGCACCCTCAATCAGTCCAGCGATGAGCTGCAGCTGAGTATGG |
| | GAAATGCCATGTTTGTCAAAGAGCAACTCAGTCTGCTGGACAGGTTCACGGAGGATGC |
| | CAAGAGGCTGTATGGCTCCGAGGCCTTTGCCACTGACTTTCAGGACTCAGCTGCAGCT |
| | AAGAAGCTCATCAACGACTACGTGAAGAATGGAACTAGGGGGAAAATCACAGATCTGA |
| | TCAACGACCTTGACTCGCAGACAATGATGGTCCTGGTGAATTACATCTTCTTTAAAGC |
| | CAAATGGGAGATGCCCTTTGACCCCCAAGATACTCATCAGTCAAGGTTCTACTTGAGC |
| | AAGAAAAAGTGGGTAATGGTGCCCATGATGAGTTTGCATCACCTGACTATACCTTACT |
| | TCCGGGACGAGGAGCTGTCCTGCACCGTGGTGGAGCTGAAGTACACAGGCAATGCCAG |
| | CGCACTCTTCATCCTCCCTGATCAAGACAAGATGGAGGAAGTGGAAGCCATGCTGCTC |
| | CCAGAGACCCTGAAGCGGTGGAGAGACTCTCTGGAGTTCAGAGAGATAGGTGAGCTCT |
| | ACCTGCCAAAGTTTTCCATCTCGAGGGACTATAACCTGAACGACATACTTCTCCAGCT |
| | GGGCATTGAGGAAGCCTTCACCAGCAAGGCTGACCTGTCAGGGATCACAGGGGCCAGG |
| | AACCTAGCAGTCTCCCAGGTGGTCCATAAGGCTGTGCTTGATGTATTTGAGGACGGCA |
| | CAGAAGCATCTGCTGCCACAGCAGTCAAAATCACCCTCCTTTCTGCATTAGTGGAGAC |
| | AAGGACCATTGTGCGTTTCAACAGGCCCTTCCTGATGATCATTGTCCCTACAGACACC |
| | CAGAACATCTTCTTCATGAGCAAAGTCACCAATCCCAAGCAAGCCTAGAGCTTGCCAT |
| | CAAGCAGTGGGGCTCTCAGTAAGGAACTTGGAATGCAAGCTGGATGCCTGGGTCTCTG |
| | GGCACAGCCTGGCCCCTGTGCACCGAGTGGCCATGGCATGTGTGGCCCTGTCTGCTTA |
| | TCCTTGGAAGGTGACAGCGATTCCCTGTGTAGCTCTCACATGCACAGGGGCCCATGGA |
| | CTCTTCAGTCTGGAGGGTCCTGGGCCTCCTGACAGCAATAAATAATTTCGTTGGAAAA |
| | AAAAAAAAAAAAAAAAAAAAAAAAAAAAAAAAAAAAC |

| | |
|---|---|
| ORF Start: at 2 | ORF Stop: TAG at 1322 |

| | | |
|---|---|---|
| SEQ ID NO: 186 | 440 aa | MW at 49553.3 kD |

| | |
|---|---|
| NOV24e, | TALESTSYTQLPEAELRMERMLPLLALGLLAAGFCPAAAdHPNSPLDEENLTQENQDR |
| CG89285-02 | |
| Protein Sequence | GTHVDLGLASANVDFAFSLYKQLVLKAPDKNVIFSPLSISTALAFLSLGAANTTLTEI |
| | LKGLKFNLTETSEAEIHQSFQHLLRTLNQSSDELQLSMGNAAFVKEQLSLLDRFTEDA |
| | KRLYGSEAFATDFQDSAAAKKLINDYVKNGTRGKITDLIKDLDSQTMMVLAAYIFFKA |
| | KWEMPFDPQDTHQSRFYLSKKKWVMVPMMSLHHLTIPYFRDEELSCTVVELKYTGNAS |
| | ALFILPDQDKMEEVEAMLLPETLKRWRDSLEFREIGELYLPKFSISRDYNLNDILLQL |
| | GIEEAFTSKADLSGITGARNLAVSQVVHKAVLDVFEEGTEASAATAVKITLLSALVET |
| | RTIVRFNRPFLMIIVPTDTQNIFFMSKVTNPKQA |

Sequence comparison of the above protein sequences yields the following sequence relationships shown in Table 24B.

TABLE 24B
Comparison of NOV24a against NOV24b through NOV24e.

| | | Identities/ |
|---|---|---|
| | | Similarities for |
| Protein | NOV24a Residues/ | the Matched |
| Sequence | Match Residues | Region |
| NOV24b | 1 . . . 423 | 376/423 (88%) |
| | 1 . . . 378 | 377/423 (88%) |
| NOV24c | 1 . . . 216 | 212/216 (98%) |
| | 1 . . . 216 | 213/216 (98%) |
| NOV24d | 1 . . . 216 | 212/216 (98%) |
| | 4 . . . 219 | 214/216 (98%) |
| NOV24e | 1 . . . 423 | 422/423 (99%) |
| | 18 . . . 440 | 423/423 (99%) |

Further analysis of the NOV24a protein yielded the following properties shown in Table 24C.

TABLE 24C
Protein Sequence Properties NOV24a

| | |
|---|---|
| PSort | 0.4600 probability located in plasma membrane; 0.1000 |
| analysis: | probability located in endoplasmic reticulum (membrane); |
| | 0.1000 probability located in endoplasmic reticulum (lumen); |
| | 0.1000 probability located in outside |
| SignalP | Cleavage site between residues 24 and 25 |
| analysis: | |

A search of the NOV24a protein against the Geneseq database, a proprietary database that contains sequences published in patents and patent publication, yielded several homologous proteins shown in Table 24D.

TABLE 24D
Geneseq Results for NOV24a

| | | NOV24a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| ABB44601 | Human wound healing related | 1 . . . 423 | 421/423 (99%) | 0.0 |
| | polypeptide SEQ ID NO 60 - | 1 . . . 423 | 422/423 (99%) | |
| | Homo sapiens, 423 aa. | | | |
| | [CA2325226-A1, 17 MAY 2001] | | | |
| AAR67259 | Alpha-1-antichymotrypsin - | 22 . . . 423 | 401/402 (99%) | 0.0 |
| | Homo sapiens, 402 aa. | 1 . . . 402 | 402/402 (99%) | |
| | [US5367064-A, 22 NOV. 1994] | | | |
| AAR82250 | Mature human wild type alpha- | 22 . . . 423 | 401/402 (99%) | 0.0 |
| | 1-antichymotrypsin - Homo | 1 . . . 402 | 402/402 (99%) | |
| | sapiens, 476 aa. [WO9527055- | | | |
| | A, 12 OCT. 1995] | | | |
| AAR83101 | Wild-type alpha-1- | 22 . . . 423 | 401/402 (99%) | 0.0 |
| | antichymotrypsin - Homo | 1 . . . 402 | 402/402 (99%) | |
| | sapiens, 402 aa. [WO9527053- | | | |
| | A1, 12 OCT. 1995] | | | |
| AAR44435 | Alpha-antichymotrypsin - Homo | 22 . . . 423 | 401/402 (99%) | 0.0 |
| | sapiens, 402 aa. [US5266465- | 1 . . . 402 | 402/402 (99%) | |
| | A, 30 NOV. 1993] | | | |

In a BLAST search of public sequence datbases, the NOV24a protein was found to have homology to the proteins shown in the BLASTP data in Table 24E.

TABLE 24E
Public BLASTP Results for NOV24a

| | | | Identities/ | |
|---|---|---|---|---|
| | | NOV24a | Similarities | |
| Protein | | Residues/ | for the | |
| Accession | | Match | Matched | Expect |
| Number | Protein/Organism/Length | Residues | Portion | Value |
| P01011 | Alpha-1-antichymotrypsin | 1 . . . 423 | 422/423 (99%) | 0.0 |
| | precursor (ACT) - Homo | 1 . . . 423 | 423/423 (99%) | |
| | sapiens (Human), 423 aa. | | | |
| AAH34554 | Serine (or cysteine) | 1 . . . 423 | 421/423 (99%) | 0.0 |
| | proteinase inhibitor, clade A | 1 . . . 423 | 423/423 (99%) | |
| | (alpha-1 antiproteinase, | | | |
| | antitrypsin), member 3 - Homo | | | |
| | sapiens (Human), 423 aa. | | | |
| ITHUC | alpha-1-antichymotrypsin | 1 . . . 422 | 415/422 (98%) | 0.0 |
| | precursor - human, 433 aa. | 1 . . . 422 | 417/422 (98%) | |
| Q9UNU9 | Alpha-1-antichymotrypsin - | 17 . . . 423 | 406/407 (99%) | 0.0 |
| | Homo sapiens (Human), 407 aa | 1 . . . 407 | 407/407 (99%) | |
| | (fragment). | | | |
| Q91WP6 | Serine protease inhibitor 2-2 - | 7 . . . 421 | 260/416 (62%) | e−144 |
| | Mus musculus (Mouse), 418 | 4 . . . 418 | 324/416 (77%) | |
| | aa. | | | |

PFam analysis predicts that the NOV24a protein contains the domains shown in the Table 24F.

TABLE 24F
Domain Analysis of NOV24a

| | | Identities/ | |
|---|---|---|---|
| | | Similarities for | |
| Pfam | NOV24a | the Matched | Expect |
| Domain | Match Region | Region | Value |
| serpin | 46 . . . 420 | 224/394 (57%) | 1.8e−216 |
| | | 345/394 (88%) | |

Example 25

The NOV25 clone was analyzed, and the nucleotide and encoded polypeptide sequences are shown in Table 25A.

TABLE 25A
NOV25 Sequence Analysis

| | |
|---|---|
| SEQ ID NO: 187 | 1860 bp |

| | |
|---|---|
| NOV25a, | GCGGATCCTCACACGACTGTGATCCGATTCTTTCCAGCGGCTTCTGCAACCAAGCGGGTCTTACCCCC |
| CG57094-01 | |
| DNA Sequence | GGTCCTCCGCGTCTCCAGTCCTCGCACCTGGAACCCCAACGTCCCCGAGAGTCCCCGAATCCCCGCTC |
| | CCAGGCTACCTAAGAGGATGAGCGGTGCTCCGACAACCAAGGAAGCCCTGATGCTCTGCGCCGCAACC |
| | GCCGTGCTACTGAGCGCTCAGGGCGGACCCGTGCAGTCCAAGTCGCCGCGCTTTGCGTCCTGGGACGA |
| | GATGAATGTCCTGGCGCACGGACTCCTGCAGCTCGGCCAGGAATGCGCGAACACCAAAGCGAACCCGC |
| | AGTCAGCTGAGCGCGCTGGAGCGCGCCTGAGCCCGTGCGGGTCCGCCTGTAAGGGAACCGAGGAATCC |
| | ACCGACCTCCCGTTAGCCCCTGAGAGCCGGGTGGACCCTGAGGTCCTTCACAGCCTGCAGACACAACT |
| | CAAGGCTCAGAACAGCAGGATCCAGCAACTCTTCCACAAGGTGGCCCAGCAGCAGCGGCACCTGGAGA |
| | AGCAGCACCTGCGAATTCAGCATCTGCAAAGCCAGTTTGGCCTCCTGGACCACAAGCACCTAGACCAT |
| | GAGGTGGCCAAGCCTGCCCGAAGAAAGAGGCTGCCCGAGATGGCCCAGCAAGTTGACCCGGCTAACAA |
| | TGTCAGCCGCCTGCACCGGCTGCCCAGGGATTGCCAGGAGCTGTTCAAGGTTAAAAAGAGGAAGAGTG |
| | GACTATTTGAAATCCAGCCTCAGGGGTCTCCGCCATTTTTGGTGAACTGCAAGATGACCTAAGATGGA |
| | GGCTGGACAGTAATTCAGAGGCGCCACGATGGCTCAGTGGACTTCAACCGGCCCTGGGAAGCCTACAA |
| | GGCGGGGTTTGGGGATCCCCACGGCGAGTTCTGGCTGGGTCTGGAGAAGGTGCATAGCATCACGGGGG |
| | ACCGCAACAGCCGCCTGGCCGTGCAGCTGCGGGACTGGGATGGCAACGCCGAGTTGCTGCAGTTCTCC |
| | GTGCACCTGGGTGGCGAGGACACGGCCTATAGCCTGCAGCTCACTGCACCCGTGGCCGGCCAGCTGGG |
| | CCCCACCACCGTCCCACCCAGCGGCCTCTCCGTACCCTTCTCCACTTGGGACAAAAATAACGACCTCC |
| | GCAGGGACAAGAACTGCGCCAAGAGCCTCTCTGGAGGCTGGTGGTTTGGCACCTGAAGCCATTCCAAC |
| | CTCAACGGCCAGTACTTCCGCTCCATCCCACAGCAGCGGCAGAAGCTTAAGAAGGGAATCTTCTGGAA |
| | GACCTGGCGGGGCCGCTACTACCCGCTGCAGGCCACCACCATGTTGATCCAGCCCATGGCAGCAGAGG |
| | CAGCCTCCTAGCGTCCTGGCTGGGCCTGGTCCCAGGCCCACGAAAGACGGTGACTCTTAACTCTGCCC |
| | GAGGATGTGGCCAAGACCACGACTGGAGAAGCCCCCTTTCTGAGTGAAGGGGGGCTGAATGCGTTGCC |
| | TCCTGAGATCGAGGCTGCAGGATATGCTCAGACTCTAGAGGCGTGGACCAAGGGGCATGGAGCTTCAC |
| | TCCTTGCTGGCCAGGGAGTTGGGGACTCAGAGGGACCACTTGGGGCCAGCCAGACTGGCCTCAATGGC |
| | GGACTCAGTCACATTGACTGACGGGACCAGGGCTTGTGTGGAATCGAGAGCGCCCTAATGGTCCTGGT |
| | GCTGTTGTGTGTAGGTCCCCTGGGACACAAGCAGGCGCCAATGGTATCTGGGCGGAAACTCACAGAGT |
| | TCTTGGAATAAAAGCAACCTCAGAACAAAAAAAAAAAAAAAAAAGCGGAGCTCACAGAGTTCTTGGAA |
| | TAAAAGCAACCTCAGAACAAAAAA |

| | |
|---|---|
| ORF Start: ATG at 154 | ORF Stop: TAG at 1369 |

| | | |
|---|---|---|
| SEQ ID NO: 188 | 405 aa | MW at 44702.1 kD |

| | |
|---|---|
| NOV25a, | MSGAPTAGAALMLCAATAVLLSAQGGPVQSKSPRFASWDEMNVLAHGLLQLGQGCANTGAHPQSAERA |
| CG57094-01 | |
| Protein | GARLSACGSACQGTEGSTDLPLAPESRVDPEVLHSLQTQLKAQNSRIQQLFHKVAQQQRHLEKQHLRI |
| Sequence | |
| | QHLQSQFGLLDHKHLDHEVAKPARRKRLPEMAQPAAPAAAASRLHRLPRDCQELFQVGERQSGLFEIQ |
| | PQGSPPFLVNCKMTSDGGWTVIQRRHDGSVDFNRPWAAYAAGFGDPHGEFWLGLEAAHSITGDRNSRL |
| | AVQLRDWDGNAELLQFSVHLGGEDTAYSLQLTAPVAGQLGATTVPPSGLSVPFSTWDQDHDLRRDKNC |
| | AKSLSGGWWFGTCSHSNLNGQYFRSIPQQRQKLKKGIFWKTWRGRYYPLQATTMLIQPAAEAAS |

| | |
|---|---|
| SEQ ID NO: 189 | 1155 bp |

| | |
|---|---|
| NOV25b, | AGATCTGGACCCGTGCAGTCCAAGTCGCCGCGCTTTGCGTCCTGAAACAAGATGAATGTCCTGGCGC |
| 17007596 | |
| DNA Sequence | ACGGACTCCTGCAGCTCGGCCAGGGGCTGCGCGAACACGCAAAGCGCACCCGAAGTAAGCTGAGCGC |
| | GCTGGAGCGGCGCCTGAGCGCGTGCGGGTCCGCCTGTCAGGGAACCGAGGGGTCCACCGACCTCCCG |
| | TTAGCCCCTGAGAGCCGGGTGGACCCTGAGGTCCTTCACAGCCTGCAGACACAACTCAAGGCTCAGA |
| | ACAGCAGGATCCAGCAACTCTTCCACAAGGTGGCCCAGCAGCAGCGGCACCTGGAGAAGCAGCACCT |
| | GCGAATTCAGCATCTGCAAAGCCAGTTTGGCCTCCTGGACCACAAGCACCTAGACAATGAGGTGGCC |
| | AAGCCTGCCCGAAGAAAGAGGCTGCCCGAGATGGCCCAGCCAGTTGACCCCGGCTAAAATGTCAGCC |
| | GCCTGCACCCGCTGCCCAGGGATTGCCAGGACCTGTTCCAGGTTAAGGAGAGGAAGAGTGGACTATT |
| | TGAAATCCAGCCTCAGGGGTCTCCGCCATTTTTGGTGAACTGCAAGATGACCTCAGATGGAGGCTGG |
| | ACAGTAATTCAGAGGCGCCACGATGGCTCAGTGGACTTCAACCGGCCCTGGGAAGCCTACAAGGCGG |
| | GGTTTGGGGATCCCCACGGCGAGTTCTGGCTAAGTCTGGAGGAGGTGAATAGAATAACGGGGGACCG |
| | CAACAGCCGCCTGGCCGTGCAGCTGCGGGACTGGGATGGCAACGCCGAGTTGCTGAAQTTCTCCGTG |
| | CACCTGGGTGGCGAGGACACCGCCTATAGCCTGCAGCTCACTGCACCCGTGGCCGGCCAGCTGGGCG |
| | CCACCACCGTCCCACCCAGCGGCCTCTCCGTACCCTTCTGGACTTGGGACCAGGATCACGACCTCCG |
| | CAGGGACAAGAACTGCGCCAAGAGCCTCTCTQGAGGCTGGTGGTTTGGAACCTGCAGCAATTCAAAC |
| | CTCAACGGCCAGTACTTCCGCTCCATCCCACAGCAGCGGCAGAAGCTTAAGAAGGGAATCTTCTGGA |
| | AGACCTGGCGGGGCCGCTACTACCCGCTGCAGGCCACCACCATGTTGATCCAGCCCATGGCAGCAGA |
| | GGCAGCCTCCCTCGAG |

| | |
|---|---|
| ORF Start: at 1 | ORF Stop: end of sequence |

| | | |
|---|---|---|
| SEQ ID NO: 190 | 385 aa | MW at 43441.5 kD |

| | |
|---|---|
| NOV25b, | RSGPVQSKSPRFASWDEMNVLAHGLLQLCGGLREHAERTRSQLSALERRLSACGSACQGTEGSTDLP |
| 170075926 | |
| Protein | LAPESRVDPEVLHSLQTQLKAQNSRIQQLFHKVAQQQRHLEKQHLRIQHLQSQFGLLDHKHLDHEVA |
| | KPARRKRLPEMAQPVDPAHNVSRLHRLPRDCQELFQVGERQSGLFEIQPQGSPPFLVNCKMTSDGGW |
| | TVIQRRHDGSVDFNRPWEAYKAGFGDPHGEFWLGLEEVHSITGDRNSRLAVQLRDWDGNAELLQFSV |
| | HLGGEDTAYSLQLTAPVAGQLGATTVPPSGLSVPFSTWDQDHDLRRDKNCAKSLSGGWWFGTCSHSN |
| | LNGQYFRSIPQQRQKLKKGIFWKTWRGRYYPLQATTMLIQPMAAEAASLE |

| | |
|---|---|
| SEQ ID NO: 191 | 1155 bp |

| | |
|---|---|
| NOV25c, | AGATCTGGACCCGTGCAGTCCAAGTCGCCGCGCTTTGCGTCCTGGGACGAGATGAATGTCCTGGCGCA |
| 164225601 | |
| DNA Sequence | CGGACTCCTGCAGCTCGGCCAGGGGCTGCGCGAACACGCGGAGCGCACCCGCAGTCAGCTGAGCGCGC |
| | TGGAGCGGCGCCTGAGCGCGTGCGGGTCCGCCTGTCAGGGAACCGAGGGGTCCACCGACCTCCCGTTA |
| | GCCCCTGAGAGCCGGGTGGACCCTGAGGTCCTTCACAGCCTGCAGACACAACTCAAGGCTCAGAACAG |
| | TTCAGCATCTGCAAAGCCAGTTTGGCCTCCTGGACCACAAGCACCTAGACCATGAGGTGGCCAAGCCT |
| | GCCCGAAGAAAGAGGCTGCCCGAGATGGCCCAGCCAGTTGACCCGGCTCACAATGTCAGCCGCCTGCA |
| | CCGGCTGCCCAGGGATTGCCAGGAGCTGTTCCAGGTTGGGGAGAGGCAGAGTGGACTATTTGAAATCC |
| | AGCCTCAGGGGTCTCCGCCATTTTTGGTGAACTGCAAGATGACCTCAGATGGAGGCTGGACAGTAATT |
| | CAGAGGCGCCACGATGGCTCAGTGGACTTCAACCGGCCCTGGGAAGCCTACAAGGCGGGGTTTGGGGA |
| | TCCCCACGGCGAGTTCTGGCTGGGTCTGGAGAAGGTGCATAGCATCACGGGGGACCGCAACAGCCGCC |
| | TGGCCGTGCAGCTGCGGGACTGGGATGGCAACGCCGAGTTGCTGCAGTTCTCCGTGCACCTGGGTGGC |
| | GAGGACACGGCCTATAGCCTGCAGCTCACTGCACCCGTGGCCGGCCAGCTGGGCGCCACCACCGTCCC |
| | ACCCAGCGGCCTCTCCGTACCCTTCTCCACTTGGGACCAGGATCACGACCTCCGCAGGGACAAGAACT |
| | GCGCCAAGAGCCTCTCTGGAGGCTGGTGGTTTGGCACCTGCAGCCATTCCAACCTCAACGGCCAGTAC |
| | TTCCGCTCCATCCCACAGCAGCGGCAGAAGCTTAAGAAGGGAATCTTCTGGAAGACCTGGCGGGGCCG |
| | CTACTACCCGCTGCAGGCCACCACCATGTTGATCCAGCCCATGGCAGCAGAGGCAGCCTCCCTCGAG |

| | |
|---|---|
| ORF Start: at 1 | ORF Stop: end of sequence |

| | | |
|---|---|---|
| SEQ ID NO: 192 | 385 aa | MW at 43440.6 kD |

| | |
|---|---|
| NOV25c, | RSGPVQSKSPRFASWDEMNVLAHGLLQLGQGLREHAERTRSQLSALERRLSACGSACQGTEGSTDLPL |
| 164225601 | |
| Protein | APESRVDPEVLHSLQTQLKAQNSRIQQLFHKVAQQQRHLEKQHLRIQHLQSQFGLLDHKHLDHEVAKP |
| Sequence | |
| | ARRKRLPEMAQPVDPAHNVSRLHRLPRDCQELFQVGERQSGLFEIQPQGSPPFLVNCKMTSDGGWTVI |
| | QRRHDGSVDFNRPWEAYKAGFGDPHGEFWLGLEKVHSITGDRNSRLAVQLRDWDGNAELLQFSVHLGG |
| | EDTAYSLQLTAPVAGQLGATTVPPSGLSVPFSTWDQDHDLRRDKNCAKSLSGGWWFGTCSHSNLNGQY |
| | FRSIPQQRQKLKKGIFWKTWRGRYYPLQATTMLIQPMAAEAASLE |

| | |
|---|---|
| SEQ ID NO: 193 | 1155 bp |

| | |
|---|---|
| NOV25d, | AGATCTGGACCCGTGCAGTCGAGTCGCCGCGCTTTGCGTCCTGGGACCAAATGAATGTCCTGGCAAC |
| 164225637 | |
| DNA Sequence | ACGGACTCCTGCAGCTCGGCCAGGGGCTGCGCGAACACGCGGAGCGCACCCGCAGTCAGCTGAGCGC |
| | GCTGGAGCGGCGCCTGAGCGCGTGCGGGTCCGCCTGTCAGGGAACCGAGGGGTCCACCGACCTCCCG |
| | TTAGCCCCTGAGAGCCGGGTGGACCCTGAGGTCCTTCACAGCCTGCAGACACAACTCAAGGCTCAGA |
| | ACAGCAGGATCCAGCAACTCTTCCACAAGGTGGCCCAGCAGCAGCGGCACCTGGAGAAGCAGCACCT |
| | GCGAATTCAGCATCTGCAAAGCCAGTTTGGCCTCCTGGACCACAAGCACCTAGACCATGAGGTGGCC |
| | AAGCCTGCCCGAAGAAAGAGGCTGCCCGAGATGGCCCAGCCAGTTGACCCGGCTCACAATGTCAGCC |
| | GCCTGCACCGGCTGCCCAGGGATTGCCAGGAGCTGTTCCAGGTTGGGGAGAGGCAGAGTGGACTATT |
| | TGAAATCCAGCCTCAGGGGTCTCCGCCATTTTTGGTGAACTGCAAGATGACCTCAGATGGAGGCTGG |
| | ACAGTAATTCAGAGGCGCCACGATGGCTCAGTGGACTTCAACCGGCCCTGGGAAGCCTACAAGGCGG |
| | GGTTTGGGGATCCCCACGGCGAGTTCTGGCTGGGTCTGGAGAAGGTGCATAGCATCACGGGGGACCG |
| | CAACAGCCGCCTGGCCGTGCAGCTGCGGGACTGGGATGGCAACGCCGAGTTGCTGCAGTTCTCCGTG |
| | CACCTGGGTGGCGAGGACACGGCCTATAGCCTGCAGCTCACTGCACCCGTGGCCGGCCAGCTGGGCG |
| | CCACCACCGTCCCACCCAGCGGCCTCTCCGTACCCTTCTCCACTTGGQACAAAAATAACGACCTCCG |
| | CAGGGACAAGAACTGCGCCAAGAGCCTCTCTGGAGGCTGGTGGTTTGGAACCTGAAGCAATTCAAAC |
| | CTCAACGGCCAGTACTTCCGCTCCATCCCACAGCAGCGGCAGAAGCTTAAGAAGGGAATCTTCTGGA |
| | AGACCTGGCGGCGCCGCCACTACCCGCTGCAGGCCACCACCATGTCGATCCAGCCCATGGCAGCAGA |
| | GGCAGCCTCCCTCGAG |

| | |
|---|---|
| ORF Start: at 1 | ORF Stop: end of sequence |

| | | |
|---|---|---|
| SEQ ID NO: 194 | 385 aa | MW at 43388.5 kD |

| | |
|---|---|
| NOV25d, | RSGPVQSKSPRFASWDEMNVLAHGLLQLGQGLREHAERTRSQLSALERRLSACGSACQGTEGSTDLP |
| 164225637 | |
| Protein | LAPESRVDPEVLHSLQTQLKAQNSRIQQLFHKVAQQQRHLEKQHLRIQHLQSQFGLLDHKHLDHEVA |
| Sequence | |
| | KPARRKRLPEMAQPVDPAHNVSRLHRLPRDCQELFQVGERQSGLFEIQPQGSPPFLVNCKMTSDGGW |
| | TVIQRRHDGSVDFNRPWEAYKAGFGDPHGEFWLGLEKVHSITGDRNSRLAVQLRDWDGNAELLQFSV |
| | HLGGEDTAYSLQLTAPVAGQLGATTVPPSGLSVPFSTWDQDHDLRRDKNCAHSLSGGWWFGTCSHSN |
| | LNGQYFRSIPQQRQKLKKGIFWKTWRGRHYPLQATTMSIQPMAAEAASLE |

| | |
|---|---|
| SEQ ID NO: 195 | 1155bp |

| | |
|---|---|
| NOV25e, | AGATCTGGACCCGTGCAGTCCAAGTCGCCGCGCTTTGCQTCCTGGGACGAGATGAATGTCCTGGCGCA |
| 170075926 | |
| DNA Sequence | CGGACTCCTGCAGCTCGGCCAGCGGCTGCGCGAACACGCGGACCGCACCCGCAGTCACCTGAGCGCGC |
| | TGGAGCGGCGCCTGAGCGCGTGCGCGTCCGCCTGTCAGGGAACCGAGGGGTCCACCGACCTCCCGTTA |
| | GCCCCTGAGAGCCGGGTGGACCCTGAGGTCCTTCACAGCCTGCAGACACAACTCAAGGCTCAGAACAG |
| | CAGGATCCAGCAACTCTTCCACAAGGTGGCCCAGCAGCAGCGGCACCTGGAGAACCAGCACCTGCGAA |
| | TTCAGCATCTGCAAAGCCAGTTTGGCCTCCTGGACCACAAGCACCTAGACCATGAGGTGGCCAAGCCT |
| | GCCCGAAQAAAGAGGCTGCCCGAGATGGCCCAGCCAGTTGACCCQGCTCACAATGTCAGCCGCCTCCA |
| | CCGGCTGCCCAQGGATTGCCAGCAGCTGTTCCAGGTTGGGGAGAGGCAGAGTGGACTATTTGAAATCC |
| | AGCCTCACGGGTCTCCGCCATTTTTGGTGAACTGCAAGATGACCTCAGATGGAGGCTGGACAGTAATT |
| | CAGACGCCCCACCATGGCTCAGTGGACTTCAACCGGCCCTGGGAAGCCTACAAGGCGGGGTTTGGGGA |
| | TCCCCACGGCGAGTTCTGCCTGGGTCTGGAGGACGTGCATAGCATCACGGGGGACCGCAACAGCCGCC |
| | TGGCCGTGCAGCTGCGGGACTGGGATGCCAACCCCCAGTTCCTLCAGTTCTCCGTGCACCTGGGTGGC |
| | GAGGACACGGCCTATAGCCTGCAGCTCACTGCACCCGTGGCCCGCCAGCTGGGCGCCACCACCGTCCC |
| | ACCCAGCQCCCTCTCCGTACCCTTCTCCACTTGGGACCACGATCACGACCTCCGCAGGGACAAGAACT |
| | GCGCCAAGAGCCTCTCTGGAQGCTGGTGGTTTGGCACCTGCAGCCATTCCAACCTCAACGGCCAGTAC |
| | TTCCGCTCCATCCCACAGCAGCGGCAGAAGCTTAAGAACGGAATCTTCTGGAAGACCTGGCGGGGCCG |
| | CTACTACCCGCTGCAGGCCACCACCATGTTGATCCAGCCCATGGCAGCAGAGGCAGCCTCCCTCGAG |

| | |
|---|---|
| ORF Start: at 1 | ORF Stop: end of sequence |

| | | |
|---|---|---|
| SEQ ID NO: 196 | 385 aa | MW at 43441.5 kD |

| | |
|---|---|
| NOV25e, | RSGPVQSKSPRFASWDEMNVLAHGLLQLGQGLREHAERTRSQLSALERRLSACGSACQGTEGSTDLPL |
| 170075926 | |
| Protein | APESRVDPEVLHSLQTQLKAQNSRIQQLPHKVAQQQRHLEKQHLRIQHLQSQFGLLDHKHLDHEVAKP |
| Sequence | |
| | ARRKRLPEMAQPVDPAHNVSRLHRLPRDCQELFQVGERQSGLPEIQPQGSPPFLVNCKMTSDGGWTVI |
| | QRRHDGSVDFNRPWEAYKAGFGDPHGEFWLGLEEVHSITGDRNSRLAVQLRDWDGNAELLQFSVHLGG |
| | EDTAYSLQLTAPVAGQLGATTVPPSGLSVPFSTWDQDHDLRRDKNCAKSLSQGWWAAGTCSHSNLNGQY |
| | FRSIPQQRQKLKKGIFWKTWRGRYYPLQATTMLIQPMAAEAASLE |

| | |
|---|---|
| SEQ ID NO: 197 | 1239 bp |

| | |
|---|---|
| NOV25f, | GACGTTAACATGAGCGGTGCTCCGACCGCCGGGGCAGCCCTGATCCTCTGCGCCGCCACCQCCGTGCT |
| 254120574 | |
| DNA Sequence | ACTGAGCGCTCAGGGCGGACCCGTGCAGTCCAAGTCGCCGCGCTTTGCGTCCTGGGACGAGATGAATG |
| | TCCTGGCGCACGGACTCCTGCAGCTCGGCCAGGGGCTGCGCGAACACGCGGAGCGCACCCGCAGTCAG |
| | CTGAGCGCGCTGGAGCGGCGCCTCAGCGCGTCCCGGTCCGCCTGTCAGGGAACCGAGGGGTCCACCGA |
| | CCTCCCGTTAGCCCCTGAGAGCCGGGTGGACCCTGAGGTCCTTCACAGCCTGCAGACACAACTCAAGG |
| | CTCAGAACAGCAGGATCCAGCAACTCTTCCACAAGGTGGCCCAGCAGCAGCGGCACCTGGAGAAGCAG |
| | CACCTGCGAATTCAGCATCTGCAAAGCCAGTTTGGCCTCCTGGACCACAAGCACCTAGACCATGAGGT |
| | GGCCAAGCCTGCCCGAAGAAAGAGGCTGCCCGAGATGGCCCACCCAGTTGACCCGGCTCACAATGTCA |
| | GCCGCCTGCACCGGCTGCCCAGGGATTGCCAGGAGCTGTTCCAGGTTGGGGAGAGGCAGAGTGGACTA |
| | TTTGAAATCCAGCCTCACGGGTCTCCGCCATTTTTGGTGAACTGCAAGATGACCTCAGATGGAGGCTG |
| | GACAGTAATTCAGAGGCGCCACGATGGCTCAGTGGACTTCAACCGGCCCTGGGAAGCCTACAAGGCGG |
| | GGTTTGGGGATCCCCACGGCGAGTTCTCGCTGGGTCTCGACAAGGTGCATAGCATCACGGGGGACCGC |
| | AACAGCCGCCTGGCCGTGCAGCTGCGGGACTGGGATGGCAACGCCGAGTTGCTGCAGTTCTCCGTGCA |
| | CCTGGGTGGCGAGGACACGGCCTATAGCCTGCAGCTCACTGCACCCGTGGCCGGCCAGCTGGGCGCCA |
| | CCACCGTCCCACCCAGCGGCCTCTCCGTACCCTTCTCCACTTGGGACCAGGATCACGACCTCCGCACC |
| | GACAAGAACTGCGCCAAGAGCCTCTCTGGAGGCTGGTGGTTTGGCACCTGCAGCCATTCCAACCTCAA |
| | CGGCCAGTACTTCCGCTCCATCCCACAGCAGCGGCAGAAGCTTAAGAAGGGAATCTTCTGGAAGACCT |
| | GGCGGGGCCGCTACTACCCGCTGCAGGCCACCACCATGTTGATCCAGCCCATGGCAGCAGAGGCAGCC |
| | TCCTAGATCAAATGGG |

| | |
|---|---|
| ORF start: at 1 | ORF Stop: TAG at 1228 |

| | | |
|---|---|---|
| SEQ ID NO: 198 | 409 aa | MW at 45542.0 kD |

| | |
|---|---|
| NOV25f, | DVNMSGAPTAGAALMLCAATAVLLSAQGGPVQSKSPRFASWDEMNVLAHGLLQLGGGLREHAERTRSQ |
| 254120574 | |
| Protein | LSALERRLSACGSACGGTEGSTDLPLAPESRVDPEVLHSLQTGLKAQNSRIQQLFHKVAQQQRHLEKQ |
| Sequence | |
| | HLRIQHLQSQFGLLDHKHLDHEVAKPARRKRLPEMAQPVDPAHNVSRLHRLPRDCGELFQVGERQSGL |
| | FEIQPQGSPPFLVNCKMTSDGGWTVIQRRHDGSVDFNRPWEAYKAGFGDPHGEFWLGLEKVHSITGDR |
| | NSRLAVQLRDWDGNAELLQFSVHLGGEDTAYSLQLTAPVACGLGATTVPPSGLSVPFSTWDQDHDLRR |
| | DKNCAKSLSGGWWFGTCSHSNLNGGYFRSIPQQRQKLKKGIFWKTWRGRYYPLQATTNLIQPMAAEAA |
| | S |

| | |
|---|---|
| SEQ ID NO: 199 | 1233 bp |

| | |
|---|---|
| NOV25g, | AGATCTACCATGAGCGGTGCTCCGACGGCCGGGGCAGCCCTGATGCTCTGCGCCGCCACCGCCGTGC |
| 254156650 | |
| DNA Sequence | TACTGAGCGCTCAGGGCGGACCCGTGCAGTCCAAGTCGCCGCGCTTTGCGTCCTGGGACGAGATGAA |
| | TGTCCTGGCGCACGGACTCCTGCAGCTCGGCCAQGGGCTGCGCGAACACGCGGACCGCACCCGCAGT |
| | CAGCTGAGCGCGCTGGAGCGGCGCCTGAGCGCGTGCGGGTCCGCCTGTCAGGGAACCGAGGGGTCCA |
| | CCGACCTCCCGTTAGCCCCTGAGAGCCGGGTGGACCCTGAGGTCCTTCACAGCCTCCAGACACAACT |
| | CAAGGCTCAGAACAGCAGGATCCAGCAACTCTTCCACAAGGTGGCCCAGCAGCAGCGGCACCTGGAG |
| | AAGCAGCACCTGCGAATTCAGCATCTGCAAAGCCAGTTTGGCCTCCTGGACCACAAGCACCTAGACC |
| | ATGAGGTGGCCAAGCCTGCCCGAAGAAAGAGGCTGCCCGAGATGGCCCAGCCAGTTGACCCGGCTCA |
| | CAATGTCAGCCGCCTGCACCGGCTGCCCAGGCATTGCCAGGAGCTGTTCCAGGTTGGGGAGAGGCAG |
| | AGTGGACTATTTGAAATCCAGCCTCAGGGGTCTCCGCCATTTTTGGTGAACTGCAAGATGACCTCAG |
| | ATGGAGGCTGGACAGTAATTCAGAGGCGCCACGATGGCTCAGTGGACTTCAACCGGCCCTGGGAAGC |
| | CTACAAGGCGGGGTTTGGGGATCCCCACGGCGAGTTCTGGCTGGGTCTGGAGAAGGTGCATAGCATC |
| | ACGGGGGACCGCAACAGCCGCCTGGCCGTGCAGCTGCGGGACTGGGATGGCAACGCCGAGTTGCTGC |
| | AGTTCTCCGTGCACCTGGGTGGCGAGGACACGGCCTATAGCCTGCAGCTCACTGCACCCGTGGCCGG |
| | CCAGCTGGGCGCCACCACCGTCCCACCCAGCGGCCTCTCCGTACCCTTCTCCACTTGGGACCAGGAT |
| | CACGACCTCCGCAGGGACAAGAACTGCGCCAAGACCCTCTCTGGAGGCTGGTGGTTTGGCACCTGCA |
| | GCCATTCCAACCTCAACGGCCAGTACTTCCGCTCCATCCCACAGCAGCCGCAGAAGCTTAAGAAGGG |
| | AATCTTCTGGAAGACCTGGCGGCGCCGCTACTACCCGCTGCACGCCACCACCATGTTGATCCAGCCC |
| | ATGGCAGCAGAGGCAGCCTCCCTCGAG |

| | |
|---|---|
| ORF Start: at 1 | ORF Stop: end of sequence |

| | | |
|---|---|---|
| SEQ ID NO: 200 | 411 aa | MW at 45800.3 kD |

| | |
|---|---|
| NOV25g, | RSTMSGAPTAGAALMLCAATAVLLSAQGGPVQSKSPRFASWDEMNVLAHGLLQLGGGLREHAERTRS |
| 254156650 | |
| Protein | QLSALERRLSACGSACGGTEGSTDLPLAPESRVDPEVLHSLQTGLKAQNSRIQQLFHKVAQQQRHLE |
| Sequence | |
| | KQHLRIQHLQSQFGLLDHKHLDHEVAKPARRKRLPEMAQPVDPAHNVSRLHRLPRDCGELFQVGERQ |
| | SGLFEIQPQGSPPFLVNCKMTSDGGWTVIQRRHDGSVDFNRPWEAYKAGFGDPHGEFWLGLEKVHST |
| | TGDRNSRLAVQLRDWDGNAELLQFSVHLGGEDTAYSLQLTAPVAGGLGATTVPPSGLSVPFSTWDQD |
| | HDLRRDKNCAKSLSGGWWFGTCSHSNLNGGYFRSIPQQRQKLKKGIFWKTWRGRYYPLQATTMLIQP |
| | MAAEAASLE |

| | |
|---|---|
| SEQ ID NO: 201 | 1239 bp |

| | |
|---|---|
| NOV25h, | TCATCCCGGGATGAGCGGTGCTCCGACGGCCGGGGCAGCCCTGATGCTCTGCGCCGCCACCGCCGTG |
| 254500366 | |
| DNA Sequence | CTACTGAGCCCTCAGGGCGGACCCGTGCAATCCAAGTCGCCGCGCTTTGCGTCCTGGGACCAGATGA |
| | ATGTCCTGGCGCACGCACTCCTGC1GCTCGGCAAGGGGCTGCGCGAACACGCGGAGCGCACCCGCAG |
| | TCAGCTGAGCGCGCTGGAGCGGCGCCTGAGCGCGTGCGGGTCCGCCTGTCAGGCAACCGACGGGTCC |
| | ACCGACCTCCCGTTAGCCCCTGACAGCCGCGTGGACCCTGAGGTCCTTCACAGCCTGCAGACACAAC |
| | TCAAGGCTCAGAACAGCAGGATCCAaCAACTCTTCCACAACGTGGCCCAGCAGCAGCGGCACCTCGA |
| | GAAGCAGCACCTGCGAATTCAGCATCTGCAAAGCCAGTTTGGCCTCCTGGACCACAAGCACCTAGAC |
| | CATGAGGTGGCCAAGCCTGCCCGAAGAAAGAGGCTGCCCGAGATGGCCCAGCCAGTTGACCCCGCTC |
| | ACAATGTCAGCCGCCTGCACCGGCTGCCCAGCGATTGCCAGGAGCTGTTCCAGGTTGGGGAGAGGCA |
| | GAGTGGACTATTTGAAATCCACCCTCAGGGGTCTCCGCCATTTTTGGTGAACTGCAAGATGACCTCA |
| | GATGGAGGCTGGACAGTAATTCAGAGGCGCCACGATGGCTCACTCGACTTCAACCGGCCCTGGGAAG |
| | CCTACAAGGCCGGGTTTGGGGATCCCCACGGCGAGTTCTGGCTGGGTCTCGAGAAGGTGCATAGCAT |
| | CACGGGGGACCGCAACAGCCGCCTGGCCGTGCAGCTGCGCGACTGGGATGGCAACGCCGAGTTGCTG |
| | CAGTTCTCCGTGCACCTGGGTGGCGAGGACACGGCCTATAGCCTGCAGCTCACTGCACCCGTGGCCG |
| | GCCAGCTGGGCGCCACCACCGTCCCACCCAGCGGCCTCTCCGTACCCTTCTCCACTTGGGACCAGGA |
| | TCACCACCTCCGCAGGGACAAGAACTGCGCCAAGAGCCTCTCTGGAGGCTCGTGGTTTGGCACCTGC |
| | AGCCATTCCAACCTCAACGGCCAGTACTTCCGCTCCATCCCACAGCAGCGGCAGAAGCTTAAGAAGG |
| | GAATCTTCTGGAAGACCTGGCGGCGCCGCTACTACCCGCTGCAGGCCACCACCATGTTGATCCAGCC |
| | CATGGCAGCAGACGCAGCCTCCCGTCCACGCGT |

| | |
|---|---|
| ORF Start: at 2 | ORF Stop: end of sequence |

| | | |
|---|---|---|
| SEQ ID NO: 202 | 413 aa | MW at 45973.6 kD |

| | |
|---|---|
| NOV25h, | HPGMSGAPTAGAALMLCAATAVLLSAQGGPVQSKSPRFASWDEMNVLAHGLLQLGQGLREHAERTRS |
| 254500366 | |
| Protein | QLSALERRLSACGSACGGTEGSTDLPLAPESRVDPEVLHSLQTGLAAGNSRIQQLFHKVAQQQRHLE |
| Sequence | |
| | KQHLRIQHLQSQFGLLDHKHLDHEVAKPARRKRLPEMAQPVDPAHNVSRLHRLPRDCGELFQVGERQ |
| | SGLFEIQPQGSPPFLVNCKMTSDGGWTVIQRRHDGSVDFNRPWEAYKAGFGDPHGEFWLGLEKVHSI |
| | HDLRRDKNCAKSLSGGWWFGTCSHSNLNCGYFRSIPQQRQKLKKGIFWKTWRGRYYPLQATTMLIQP |
| | MAAEAASRRRX |

| | |
|---|---|
| SEQ ID NO: 203 | 1167 bp |

| | |
|---|---|
| NOV25i, | GACGTTAACATGGGACCCGTGCAGTCCAAGTCGCCGCGCTTTGCGTCCTGAAACGAAATGAATGTCCT |
| 226679956 | |
| DNA Sequence | GGCGCACGGACTCCTGCAGCTCGGCCAGGGGCTGCGCGAACACGCGGAGCGCACCCGCAGTCAGCTGA |
| | GCGCGCTGGAGCGGCGCCTGAGCGCGTGCGGGTCCGCCTGTAAGGGAACCGAGAAGTCAACCGACCTC |
| | CCGTTAGCCCCTGAGAGCCGGGTGGACCCTGAGGTCCTTCACAGCCTGCAGACACAACTCAAGGCTCA |
| | GAACAGCAGGATCCAGCAACTCTTCCACAAGGTGGCCCAGCAGCAGCGGCACCTGGAGAAGCAGCACC |
| | TGCGAATTCAGCATCTGCAAAGCCAGTTTCGCCTCCTGCACCACAAGAACCTAGACAATGAGGTGCCC |
| | AAGCCTGCCCGAAGAAAGAGGCTGCCCGAGATGGCCCAGCCAGTTGACCCGGCTCACAATGTCAGCCG |
| | CCTGCACCGGCTGCCCAGGGATTGCCAGGAGCTGTTCCAGGTTGGGGAGAGGCAGAGTGGACTATTTG |
| | AAATCCAGCCTCAGGGGTCTCCGCCATTTTTGGTGAACTGCAAGATGACCTCAGATGGAGGCTGGACA |
| | GTAATTCAGAGGCGCCACGATGGCTCAGTGGACTTCAACCGGCCCTGGGAAGCCTACAAGGCGGGGTT |
| | TGGGGATCCCCACGGCGAGTTCTGGCTGGGTCTGGAGAAGGTGCATAGCATCACGGGGGACCGCAACA |
| | GCCGCCTGGCCGTGCAGCTGCGGGACTGGAATGGCAACGCCGAGTTGCTGAAGTTCTCCGTGCACCTG |
| | GGTGGCGAGGACACGGCCTATAGCCTGCAGCTCACTGCACCCGTGGCCGGCCAGCTGGGCGCCACCAC |
| | CGTCCCACCCAGCGGCCTCTCCGTACCCTTCTCCACTTGGGACCAAAATCACGACCTCCAAACGGAAA |
| | AGAACTGCGCCAAGAGCCTCTCTGGAGGCTGGTGGTTTGGCACCTGCAGCCATTCCAACCTCAACGGC |
| | CAGTACTTCCGCTCCATCCCACAGCAGCGGCAGAAGCTTAAGAAGGGAATCTTCTGGAAGACCTGGCG |
| | GGGCCGCTACTACCCGCTGCAGGCCACCACCATGTTGATCCAGCCCATGGCAGCAGAGGCAGCCTCCT |
| | AGATCGATGGG |

| | |
|---|---|
| ORF Start: at 1 | ORF Stop: TAG at 1156 |

| | | |
|---|---|---|
| SEQ ID NO: 204 | 385 aa | MW at 43414.6 kD |

| | |
|---|---|
| NOV25i, | DVNMGPVQSKSPRFASWDEMNVLAHGLLQLGGGLREHAERTRSQLSALERRLSACGSACGGTEGSTDL |
| 226679956 | |
| Protein | PLAPESRVDPEVLHSLQTGLKAGNSRTGGLFHKVAGQQRHLEKQHLRIQHLQSQFGLLDHAALDHEVA |
| Sequence | |
| | KPARRKRLPEMAQPVDPAHNVSRLHRLPRDCGELFQVGERQSGLFEIQPQGSPPFLVNCAATSDGGWT |
| | VIQRRHDGSVDFNRPWEAYKAGFGDPHGEFWLGLEKVHSITGDRNSRLAVQLRDWDGNAELLQFSVHL |
| | GGEDTAYSLQLTAPVAGGLQATTVPPSGLSVPFSTWDQDHDLRRDKNCAKSLSGGWWFQTCSHSNLNG |
| | PQQRQKLKKGIFWKTWRGRYYPLQATTMLIQPMAAEAAS |

| | |
|---|---|
| SEQ ID NO: 205 | 1187 bp |

| | |
|---|---|
| NOV25j, | GACGTTAACATGGGACCCGTGCAGTCCAAGTCGCCGCGCTTTGCGTCCTGGGACGAGATGAATGTCCT |
| 254500319 | |
| DNA Sequence | GGCGCACGGACTCCTGCAGCTCGGCCAGGGGCTGCGCGAACACGCGGAGCGCACCCGCAGTCAGCTGA |
| | GCGCGCTGGAGCGGCGCCTGAGCGCGTGCGGGTCCGCCTGTCAGGGAACCGAGGGGTCCACCGACCTC |
| | CCGTTAGCCCCTGAGAGCCGGGTGGACCCTGAGGTCCTTCACAGCCTGCAGACACAACTCAAGGCTCA |
| | GAACAGCAGGATCCAGCAACTCTTCCACAAGGTGGCCCAGCAGCAGCGGCACCTGGAGAAGCAGCACC |
| | AAGCCTGCCCCGAAGAAAGAGGCTGCCCGAGATGGCCCAGCCAGTTGACCCGGCTCAAATGTCAGCCG |
| | CCTGCACCGGCTGCCCAGGGATTGCCAGGAGCTGTTCCAAATTGGGGAGAAAAAGAGTAAACTATTTG |
| | AAATCCAGCCTCAGGGGTCTCCGCCATTTTTGGTGAACTGCAAGATGACCTCAGATGGAGGCTGGACA |
| | GTAATTCAGAGGCGCCACGATGGCTCAGTGGACTTCAACCGGCCCTGGGAAGCCTACAAGGCGGGGTT |
| | TGGGGATCCCCACGGCGAGTTCTGGCTGGGTCTGGAGAAGGTGCATAGCATCACGGGGGACCGCAACA |
| | GCCGCCTGGCCGTGCAGCTGCGGGACTGGGATGGCAACGCCGAGTTGCTGCAGTTCTCCGTGCACCTG |
| | GGTGGCGAGGACACGGCCTATAGCCTGCAGCTCACTGCACCCGTGGCCGGCCAGCTGGGCGCCACCAC |
| | CGTCCCACCCAGCGGCCTCTCCGTACCCTTCTCCACTTGGGACCAGGATCACGACCTCCGCAGGGACA |
| | AGAACTGCGCCAAGAGCCTCTCTGGAGGCTGGTGGTTTGGCACCTGCAGCCATTCCAACCTCAACGGC |
| | CAGTACTTCCGCTCCATCCCACAGCAGCGGCAGAAGCTTAAGAAGGGAATCTTCTGGAAGACCTGGCG |
| | GGGCCGCTACTACCCGCTGCAGGCCACCACCATGTTGATCCAGCCCATGGCAGCAGAGGCAGCCTCCT |
| | AGATCGATGGGAAGGGCGAATTCTGCAGATA |

| | |
|---|---|
| ORF Start: at 1 | ORF Stop: TAG at 1156 |

| | | |
|---|---|---|
| SEQ ID NO: 206 | 385 aa | MW at 43414.6 kD |

| | |
|---|---|
| NOV25j, | DVNMGPVQSKSPRFASWDEMNVLAHGLLQLGQGLREHAERTRSQLSALERRLSACGSACQGTEGSTDL |
| 254500319 | |
| Protein | PLAPESRVDPEVLHSLQTGLKAGNSRIQQLFHKVAGQQRHLEKQHLRIQHLQSQFGLLDHKHLDHEVA |
| Sequence | |
| | KPARRKRLPEMAQPVDPAHNVSRLHRLPRDCQELFQVGERQSGLFEIQPQGSPPFLVNCKMTSDGGWT |
| | VIQRRHDGSVDFNRPWEAYKAGFGDPHGEFWLGLEKVHSITGDRNSRLAVQLRDWDQNAELLQFSVHL |
| | GGEDTAYSLQLTAPVAGGLGATTVPPSGLSVPFSTWDQDHDLRRDKNCAKSLSGGWWFGTCSHSNLNG |
| | QYFRSIPQQRQKLKKGIFWKTWRGRYYPLQATTMLIQPMAAEAAS |

| | |
|---|---|
| SEQ ID NO: 207 | 1167 bp |

| | |
|---|---|
| NOV25k, | TCATCCCGGGATGGGACCCGTGCAGTCCAAGTCGCCGCGCTTTGCGTCCTGGGACGAGATGAATGTC |
| 254500445 | |
| DNA Sequence | CTGGCGCACGGACTCCTGCAGCTCGGCCAGGGGCTGCGCGAACACGCGGAGCGCACCCGCAGTCAGC |
| | TGAGCGCGCTGGAGCGGCGCCTGAGCGCGTGCGGGTCCGCCTGTCAGGAAACCGAGGGGTCAACCGA |
| | CCTCCCGTTAGCCCCTGAGAGCCGGGTGGACCCTGAGGTCCTTCACAGCCTGCAGACACAACTCAAG |
| | GCTCAGAACAGCAGGATCCAGCAACTCTTCCACAAGGTGGCCCAGCAGCAGCGGCACCTGAAGAAGC |
| | ACACCTGCGAATTCAGCATCTGCAAAAGCCAGTTTGGCCTCCTGGACCACAAGAACCTAGACCATGA |
| | GGTGGCCAAGCCTAACCCGAAGAAAGAGGCTGCCCGAGATGGCCCAGCCAGTTAACCCGGCTAAAAT |
| | GTCGCCGCCTGCACCGGCTGCCCAGGGATTGCCAGGAAACTCTTCCAGGTTGGAAAAAGGAAGAGTG |
| | GACTATTTGAAATCCAGCCTCAGGGGTCTCCGCCATTTTTGGTGAACTGCAAGATGACCTAAGATGG |
| | AGGCTGGACAGTAATTCGACGCCCCACGATGGCTCAGTCGACTTCAACCGGCCCTCAAGAAGCCTAC |
| | AAGGCGGGGTTTGGGGATCCCCACGGCGAGTTCTGGCTGGGTCTGGAGAAGGTGCATAGCATCACGG |
| | GGGACCGCAACAGCCGCCTGGCCGTGCAGCTGCGCGACTGGGATGGCAACGCCGAGTTGCTGCAGTT |
| | CTCCGTGCACCTGGGTGGCGACGACACGGCCTATAAACCTGCAGCTCACTCAACCCGTGGCCGGCAG |
| | CTCGGCGCCACCACCGTCCCACCCAGCGGCCTCTCCGTACCCTTCTCAACTTAAGACCAGGATAACG |
| | ACCTCCGCAGGGACAAGAACTGCGCCAAGAGCCTCTCTCGAGGCTGGTGGTTTGGAACCTGAAGCCA |
| | TTCCAACCTCAACCGCCAGTACTTCCGCTCCATCCCACAGCAGCGGCAGAAGCTTAAGAAGAAAATC |
| | TTCTGGAAGACCTGGCGGGGCCGCTACTACCCGCTGCAGGCCACCACCATGTTGATCCAGCCCATGG |
| | CAGCAGAGGCAGCCTCCCGTCCACGCGT |

| | |
|---|---|
| ORF Start: at 2 | ORF Stop: end of sequence |

| | | |
|---|---|---|
| SEQ ID NO: 208 | 389 aa | MW at 43846.1 kD |

| | |
|---|---|
| NOV25k, | HPGMGPVQSKSPRFASWDEMNVLAHGLLQLGGGLREHAERTRSQLSALERRLSACGSACGGTEGSTD |
| 25450045 | |
| Protein | LPLAPESRVDPEVLHSLQTGLKAGNSRIQQLFHKVAGQQRHLEKQHLRIQHLQSQFGLLDHAALDHE |
| Sequence | |
| | VAKPARRKRLPEMAQPVDPAHNVSRLHRLPRDCQELFQVGERQSGLFEIQPQGSPPFLVNCKMTSDG |
| | GWTVIQRRHDGSVDFNRPWEAYKAGFGDPHGEFWLGLEKVHSITGDRNSRLAVQLRDWDGNAELLQF |
| | SVHLGGEDTAYSLQLTAPVAGGLGATTVPPSGLSVPFSTWDQDHDLRRDKNCAKSLSGGWWFGTCSH |
| | SWLNGGYFRSIPQQRQKLKKGIFWKTWRGRYYPLQATTMLIQPMAAEAASRRRX |

| | |
|---|---|
| SEQ ID NO: 209 | 738 bp |

| | |
|---|---|
| NOV25l, | AGATCTCTGCCCGAGATGGCCCAGCCAGTTGACCCGGCTCACAATGTCAGCCGCCTGCACCGGCTGCC |
| 248210290 | |
| DNA Sequence | CAGGGATTGCCAGGAGCTGTTCCAGGTTGGGGAGAGGCAGAGTGGACTATTTGAAATCAAGCCTAAGG |
| | GGTCTCCGCCATTTTTGGTGAACTGCAAGATGACCTCAGATGGAGGCTGGACAGTAATTCAGAGGCGC |
| | CACGATGGCTCAGTGGACTTCAACCGGCCCTGGAGAGCCTACAAGGCGGGGTTTGAAGATCCCAACGG |
| | CGAGTTCTGGCTGGGTCTCGAGAAGGTCCATAGCATCACGGGGGACCGCAACAGCCGCCTGGCCGTGC |
| | AGCTGCGGGACTGGGATGGCAACGCCGAGTTGCTGCAGTTCTCCGTGCACCTGGGTGGCGAGGACACG |
| | GCCTATAGCCTGCAGCTCACTGCACCCGTGCCGGCCAGCTGAACGCCACCACCGTCCAACCAAGCACG |
| | CCTCTCCGTACCCTTCTCCACTTGGGACCAGGATCACGACCTCCGCAGGGACAAGGAACTGCGCAAGA |
| | CCCTCTCTGGACGCTGGTGGTTTGGCACCTGCAACCATTCCAACCTCAACGGCAAGTACTTCCGCTCC |
| | ATCCCACAGCAGCGGCAGAACCTTAAGAAGGGAATCTTCTGGAACACCTGGCAAGGCCGCTACTACCC |
| | GCTGCAGGCCACCACCATGTTGATCCAGCCCATGGCAGCAGAGGCAGCCTCCCTCAAG |

| | |
|---|---|
| ORF Start: at 1 | ORF Stop: end of sequence |

| | | |
|---|---|---|
| SEQ ID NO: 210 | 246 aa | MW at 27677.9 kD |

| | |
|---|---|
| NOV25l, | RSLPEMAQPVDPAHNVSRLHRLPRDCQELFQVGERQSGLFEIQPQGSPPFLVNCKMTSDGGWTVIQRR |
| 248210290 | |
| Protein | HDGSVDFNRPWEAYKAGFGDPHGEFWLGLEKVHSITGDRNSRLAVQLRDWDGNAELLQFSVHLGGEDT |
| Sequence | |
| | AYSLQLTAPVAGGLGATTVPPSGLSVPFSTWDQDHDLRRDKNCAKSLSGGWWFGTCSHSNLNGGYFRS |
| | IPQQRQKLKKGIFWKTWRGRYYPLQATTMLIQPMAAEAASLE |

| | |
|---|---|
| SEQ ID NO: 211 | 1218 bp |

| | |
|---|---|
| NOV25m, | AGATCTGGACCCGTGCAGTCCAAGTCGCCGCGCTTTGCGTCCTGGGACGAGATGAATGTCCTGGCGC |
| 25251418 | |
| DNA Sequence | ACGGACTCCTGCAGCTCGGCCAGGGGCTGCGCGAACACGCGGAGCCAACCCGCAGTAAGCTGAGCGC |
| | GCTGGAGCGGCGCCTGAGCGCGTGCGGGTCCGCCTGTCAGGGAACCGAGGGGTCCACCGACCTCCCG |
| | TTAGCCCCTGAGAGCCGGGTGGACCCTGAGGTCCTTCACAGCCTGCAGACACAACTCAAGGCTCAGA |
| | ACAGCAGGATCCAGCAACTCTTCCACAAGGTGGCCCAGCAGCAGCGGCACCTGGAGAAGCAGCACCT |
| | GCGAATTCAGCATCTGCAAAGCCAGTTTGGCCTCCTGGACCAAAAGAACCTAGACCATGAGGTGGCC |
| | AAGCCTGCCCGAGAAAGAGGAAGGCTGCCCGAGATGGCCCAGCAAGTTGACCCAACTAATGTAAGCC |
| | GCCTGCACCGGCTGGCCCAGGGATTGCCAGGAGCTGTTCAATGTTGAAAGAAAAAGAGTGGACTATT |
| | TGAAATCCAGCCTCAGGGGTCTCCGCCATTTTTGGTGAACTGCAAGATGACCTCAGATGGAGGCTGG |
| | ACAGTAATTCAGAGGCGCCACGATGGCTCAGTGGACTTCAACCGGCCCTGGGAAGCCTACAAGGCGG |
| | GGTTTGGGGATCCCCACGGCGAGTTCTGGCTGGGTCTGGAGAAGGTGCATAGCATCACGGGGGACCG |
| | CAACAGCCGCCTGGCCGTGCAGCTGCGGGACTGGCATGGCAACGCCAAGTTGCTGCAGTTCTCCGTG |
| | CACCTGGGTGGCGAGGACACGGCCTATAGCCTGCAGCTCACTGCACCCGTGGCCGGCCAGCTGGGCG |
| | CCACCACCGTCCCACCCAGCGGCCTCTCCGTACCCTTCTCCACTTGGGACCAGGATCACGACCTCCG |
| | CAGGGACAGAGGAACTGCGCCAAGAGCCTCTCTGCCCCATCGGTGGCTAAGACCTGACAATGTTCCC |
| | TCTCCCCTGACCCCGGCAGGAGGCTGGTGGTTTGGCACCTGCAGCCATTCCAACCTCAACGGCCAGT |
| | ACTTCCGCTCCATCCCACAGCAGCGGCAGAAGCTTAAGAAAAGAATCTTCTGGAAGACCTGGCGGGG |
| | CCGCTACTAGCCCGCTGCAGGCCACCACCATGTTGATCCGCCAATGGCAGAAGAGGAAGCCTCCCTC |
| | GAGAAGGGCGAA |

| | |
|---|---|
| ORF Start: at 1 | ORF Stop: end of sequence |

| | | |
|---|---|---|
| SEQ ID NO: 212 | 406 aa | MW at 45586.0 kD |

| | |
|---|---|
| NOV25m, | RSGPVQSKSPRFASWDEMNVLAHGLLQLGGGLREHAERTRSQLSALERRLSACGSACGGTEGSTDLP |
| 252514148 | |
| Protein | LAPESRVDPEVLHSLQTGLKAGNSRIQQLFHKVAGQQRHLEKQHLRIQHLQSOFGLLDHKHLDHEVA |
| Sequence | |
| | KPARRKRLPEMAGPVDPAHNVSRLHRLPRDCGELFHGERQSGLFEIQPQGSPPFLVAACAATSDGGW |
| | TVIQRRHDGSVDFNRPWEAYKAGFGDPHGEFWLGLEKAASTTGDRNSRLAVQLRDWDGNAELLQFSV |
| | HLGGEDTAYSLQLTAPVAGQLGATTVPPSGLSVPFSTWDQDHDLRRDKNCAKSLSAPSVAQRPDHVP |
| | SPLTPACGWWFGTCSHSNLNGGYFRSIPQQRQKLKKGIFWKTWRGRYYPLQATTMLIQPMAAEAASL |
| | EKGE |

| | |
|---|---|
| SEQ ID NO: 213 | 1223 bp |

| | |
|---|---|
| NOV25n, | CAGAATTCGCCCTTAGATCTGGACCCGTGCAGTCCAAGTCGCCGCGCTTTGCGTCCTGGGACGAGAT |
| 252514189 | |
| DNA Sequence | GAATGTCCTGGCGCACGGACTCCTGCAGCTCGGCCAGGGGCTGCGCGAACACGCGGACCCAACCCGC |
| | AGTCAGCTGAGCGCGCTGGAGCGGCGCCTGAGCGCGTGCAAAGTCCGCCTGTAAGGAACCGAGGGGT |
| | CCACCGACCTCCCGTTAGCCCCTGAGAGCCGGTGGACCCTGACGTCCTTAAAAGCCTGCAGAGCACA |
| | ACTCAAGGCTCAGAACAGCAGGATCCAGCAACTCTTCCACAAGGTGGCCCAGCAGCAGCGGCACCTG |
| | GAGAGCAGCACCTGCGAATTCAGCATCTGCAAAGCCGTTTGGCCTCCTAAACCAGGAAAGAACCTAG |
| | ACCATGAGGTGGCCAAGCCTGCCCGAAGAAAGAGGCTGCCCGAGATGGCCAAGCAAGTTGACCCGGC |
| | TCACAATGTCAGCCGCCTGCACCGGCTGCCCAGGGATTGCCGAGCTGTTCGAAAGGTTGGAAAGAGG |
| | CAGAGTGGACTATTTGAAATCCAGCCTCAGGGGTCTCCGCAATTTTTGGTGAAACTGAAGATGACCT |
| | CAGATGGAGGCTGGACAGTAATTCAGAGGCGCCACGATGGCTCAGTGGACTTCAACCGGCCCTGGGA |
| | AGCCTACAGGCGGGGTTTGGGAGATCCCCACGGCGAGTTCTGGCTAAGTCTGGAGAAGGTGAATAGC |
| | ATCATGGGGGACCGCAACAGCCGCCTGGCCGTGCAGCTGCGGGACTGGGATGGAAACGCCGAGTTGC |
| | TGCAGTTCTCCGTGCACCTGGGTGGCGAGGACACGGCCTATAGCCTGGAGCTAACTGAACCCGTGGC |
| | CGGCCAGCTGGGCGCCACCACCGTCCCACCCAGCGGCCTCTCCGTACCCTTCTCCACTTGGGACCAG |
| | GATCACGACCTCCGCAGGGACAAGAACTGCGCCAAGAGCCTCTCTGCCCCATCAATAACTCAAAGAC |
| | CTGACCATGTTCCCTCTCCCCTGACCCCGGCAGGAGGCTGGTGGTTTGGCACCTGCAGCCATTCCAA |
| | CCTCACGGCCGTACTTCCGCTCCATCCCACAGCAGCGGAAGAAAGCTTAAGAAGGGAATCTTCTGG |
| | AAGACCTGGCGGGGCCGCTACTACCCGCTGCAGGCCACCACCATGTTGATCCAGCCCATGGCAGCAG |
| | AGGCAGCCTCCCTCGAG |

| | |
|---|---|
| ORF Start: at 3 | ORF Stop: end of sequence |

| | | |
|---|---|---|
| SEQ ID NO: 214 | 407 aa | MW at 45753.2 kD |

| | |
|---|---|
| NOV25n, | EFALRSGPVQSKSPRFASWDEMNVLAHGLLQLGQGLREHAERTRSQLSALERRLSACGSACQGTETS |
| 252514189 | |
| Protein | TDLPLAPESRVDPEVLHSLQTQLKAQNSRIQQLFHKVAQQQRHLEKQHLRIQHLQSQFGLLDHKHLD |
| Sequence | |
| | HEVAKPARRKRLPEMAQPVDPAHNVSRLHRLPRDCQELFQVGERQSGLFEIQPQGSPPFLVNCKMTS |
| | DGGWTVIQRRHDGSVDFNRPWEAYKAGFGDPHGEFWLGLEKVHSIMGDRNSRLAVQLRDWDGNAELL |
| | QFSVHLGGEDTAYSLQLTAPVAGGLGATTVPPSGLSVPFSTWDQDHDLRRDKNCAKSLSAPSVAQRP |
| | DHVPSPLTPAGGWWFGTCSHSNLNGGYFRSIPQQRQKLKKGIFWKTWRGRYYPLQATTMLIQPMAAE |
| | F |

| | |
|---|---|
| SEQ ID NO: 215 | 1041 bp |

| | |
|---|---|
| NOV25o, | AGATCTGGACCCGTGCAGTCCAAGTCGCCGCGCTTTGCGTCCTGGGACGAGATGAATGTCCTGGCGC |
| 252514198 | |
| DNA Sequence | ACGGACTCCTGCAGCTCGGCCAGGGGCTGCGCGAACACGCGGAGCGCACCCGCAGTCAGCTGAGCGC |
| | GCTGGAGCGGCGCCTGAGCGCGTGCGGGTCCGCCTGTCAGGGAACCGAGGGGTCCACCGACCTCCCG |
| | TTAGCCCCTGAGAGCCGGGTGGACCCTGAGGTCCTTCACAGCCTGCAGACACAACTCAAGGCTCAGA |
| | ACAGCAGGATCCAGCAACTCTTCCACAAGGTGGCCCAGCAGCAGCGGCACCTGGAGAAGCAGCACCT |
| | GCGAATTCAGCATCTGCAAAGCCAGTTTGGCCTCCTGGACCACAAGCACCTAGACCATGAGGTGGCC |
| | AAGCCTGCCCGAAGAAACAGGCTGCCCGAGATGGCCCAGCCAGTTGACCCGGCTAAAAATGTAAGCC |
| | GCCTGCACCATGGAGGCTGGACAGTAATTCAGAGGCGCCACGATGGCTCAGTGGACTTCAACCGGCC |
| | CTGGGAAGCCTACAACGCGGGGTTTGGGGATCCCCACGGCGAGTTCTAACTGGGTCTGGAGAAGGTG |
| | CATAGCATCATGGGGGACCGCAACAGCCGCCTGGCCGTGCAGCTAACGGAACTGGATGGAAACGCCG |
| | AGTTGCTGCAGTTCTCCGTGCACCTGGGTGGCGAGCACACAACCTATAGCCTGAAGCTAACTGAACC |
| | CGTGGCCGCCCAGCTGGGCGCCACCACCGTCCCACCCAGCGGCCTCTCCGTACCCTTCTCAACTTGG |
| | GACCAGGATCACGACCTCCGCAGGGACAAGAACTGCGCCAAGAGCCTCTCTGGAGGCTAATAATTTG |
| | GCACCTGCAGCCATTCCAACCTCAACGGCCAGTACTTCCGCTCCATCCCACAGCAGCGGCAGAAGCT |
| | TAAGAGGGAATCTTCTGGAAGACCTGGCGGGGCCGCTACTACCCGCTGCAAGGCCACCACAATGTTG |
| | ATCCAGCCCATGGCAGCAGAGCCAGCCTCCCTCGAG |

| | |
|---|---|
| ORF Start: at 1 | ORF Stop: end of sequence |

| | | |
|---|---|---|
| SEQ ID NO: 216 | 347 aa | MW at 39173.8 kD |

| | |
|---|---|
| NOV25o, | RSGPVQSKSPRFASWDEMNVLAHOLLQLGGGLREHAERTRSQLSALERRLSACGSACGGTEGSTDLP |
| 252514198 | |
| Protein | LAPESRVDPEVLHSLQTGLKAGNSRIQQLFHKVAGQQRHLEKQHLRIQHLQSQFGLLDHAALDHEVA |
| Sequence | |
| | HSIMGDRNSRLAVQLRDWDGNAELLQFSVILGGEDTAYSLQLTAPVAGGLGATAAPPSGLSVPFSTW |
| | KPARRKRLPEMAQPVDPAHNVSRLHHGGWTVIQRRHDGSVDFNRPWEAYKAGFGDPHGEFWLGLEKV |
| | DQDHDLRRDKNCAKSLSGGWWFGTCSHSNLNGGYFRSIPQQRQKLKKGIFWKTWRGRYYPLQATTML |
| | IQPMAAEAASLE |

| | |
|---|---|
| SEQ ID NO: 217 | 1209 bp |

| | |
|---|---|
| NOV25p, | AGATCTGGACCCGTGCAGTCCAAGTCGCCGCGCTTTGCGTCCTGGGACGAGATGAATGTCCTGGCGC |
| 252514198 | |
| DNA Sequence | ACGGACTCCTGCAGCTCGGCCAGGGGCTGCGCGAACACGCGGAGCGAACCCGAAGTAAGCTAAGCGC |
| | GCTGGAGCGGCGCCTGAGCGCGTGCGGGTCCGCCTGTCAGGGAACCGAAAAATCGACCGACCTCCCG |
| | TTAGCCCCTGAGAGCCGGGTGGACCCTGAGGTCCTTCACAGCCTGCAGACACAACTCAAGGCTGAGA |
| | ACAGCAGGATCCAGCAACTCTTCCACAAGGTGGCCCAGCAGCAGCGGCACCTGGAGAAGCAGCACCT |
| | GCGAATTCAGCATCTGCAAGCGCAGTTTGGCCTCCTGGACCACAAGCACCTAGACGATGAGGTGGCC |
| | AGCCTGCCCGAAGAAAGAGGCTGGCCCGAGATGGCCCAGCCAGTTGACCCGGCTCAGAATGTGAGCC |
| | GCCTGACCGGCTGCCCGGGATTGGGCCAGGAGCTGTTCCAGGTTGGGGAGAGGGAGAGTGGACTATT |
| | TGAAATCCAGCCTCAGGGTCTCCGCGGCATTTTTGGTGAACTGAAGATGACCTGAGATAAAGGCTGG |
| | ACAGTAATTCAGAGGCGCCACGATGGCTCAGTGGACTTCAACCGGCCCTGAAAAGCCTACAAGGCGG |
| | GGTTTGGGGATCCCCACGGCGAGTTCTGGGCTGGGTCTGGAGAAGGTGGATAGGATGACGGAAACCG |
| | CAACAGCCGCCTGGCCGTGCAGCTGCGGGACTGGGATGGCAACGCCGAGTTGCTGGAGTTCTCCGTG |
| | CACCTGGGTGGCGAGGACACGGCCTATAGCCTGCAGCTCACTGCACCCGTGGCCGGCCAGCTGGGCG |
| | CCACCACCGTCCCCCCAGCGGCCTCTCCGTAACCCTTCTCCACTTGGGACGAGGATGACGACCTCCG |
| | CAGGGACAAGAACTGCGCCAGAGCCTCTCTGGAGCCCCATCGGTAACTCAGACCTGACGATGTTCCC |
| | TCTCCCCTGACCCCGGCAGGAGGCTGGTGGTTTGGAGCACCTGCAGCGATTCAACCTAACGGCGAGT |
| | ACTTCCGCTCCATCCCACAGCAGCGGCAGAAGCTTAAGAAGGGAATCTTCTGGAAGACCTGGCGGGG |
| | CCGCTACTACCCGCTGCAGGCCACCACCATGTTGATCCAGCCGATAAGAGGAGAGGGAGCCTCCCTC |
| | GAG |

| | |
|---|---|
| ORF Start: at 1 | ORF Stop: end of sequence |

| | | |
|---|---|---|
| SEQ ID NO: 218 | 403 aa | MW at 45262.6 kD |

| | |
|---|---|
| NOV25p, | RSGPVQSKSPRFASWDEMNVLAHGLLQLGGGLREHAERTRSQLSALERRLSACGSACGGTEGSTDLP |
| 252514202 | |
| Protein | LAPESRVDPEVLHSLQTQLKAQNSRIQQLFHKVAQQQRHLEKQHLRIQHLQSQFGLLDHKHLDHEVA |
| Sequence | |
| | KPARRKRLPEMAGPVDPAIVSRLHRLPRDCGELFQVGERQSGLFEIQPQGSPPAALAACAATSDGGW |
| | TVIQRRHDGSVDFNRPWEAYKAGFGDPHGEFWLGLEKVHSITGDRNSRLAVQLRDWDGNAELLQFSV |
| | HLGGEDTAYSLQLTAPVAGQLGATTVPPSGLSVPFSTWDQDHDLRRDKNCAKSLSAPSVAQRPDHVP |
| | SPLTPAGGWWFGTCSHSNLNGGYFRSIPQQRQIKKGIAAWKTWRGRYYPLQATTMLIQPMAAEAASL |
| | E |

| | |
|---|---|
| SEQ ID NO: 219 | 1258 bp |

| | |
|---|---|
| NOV25q, | AAGGCTCCGCGGCCGCCCCCTTCACCATGAGCGGTGCTCGACGGCCGGGGCAGCCCTGATGCTCTGC |
| 228039766 | |
| DNA Sequence | GCCGCCACCGCCGTGCTACTGAGCGCTCAGGGCGGACCCGTGCAGTCCAGTCAACCGCGCTTTGCGT |
| | CCTGGGACGAGATGATGTCCTAAGCGCACGGACTCCTGCAGCTCGGCCAGGGGCTGCGCGAACACGC |
| | GGAGCGCACCCGCAGTCAGCTGAGCGCGCTGGAGCGGCGCCTGAGCGCGTGCGGGTCCGCCTGTCAG |
| | GGAACCGAGGGGTCAGCCGACCTCCCGTTAGCCCCTGAGAGCCGGGTGGACCCTGAGGTCCTTCACA |
| | GCCTGCAGACACACTCGAAGGCTCAGAACAGCAGGATCCAGCAACTCTTCCACAAGGTGGCCCAGCA |
| | CCAGCGGCGACCTGGAGAAGCAGCACCTGCGATTCAGCATCTGCAAACCCAGTTTGGCCTCCTGGAC |
| | CACAAGCACCTAGAACCATGAGGTGGCCAAGCCTGCCCGAGAAAGAGGCTGCCCGAGATGGCCCAGC |
| | CAGTTGACCCCGCTCACATGTCAGCCGCCTGCACCGAACTGCCCAGGGATTGCCAGGAGCTGTTCCA |
| | GGTTGGGGAGAGAACAGAGTGGACTATTTGAATCCAGCCTCAGGCGTCTCCGCCATTTTTGGTGAAC |
| | TGCAAGATGACCTCAGATGGAGGCTGGACAGTAATTCAGAGGCGCCACGATCGCTCACTCGACTTCA |
| | ACCGGCCCTGGGAAGCCTACAAGGCGGGGTTTGGCGATCCCCACGGCGAGTTCTGGCTGGGTCTGGA |
| | GAAGGTCCATAGCATCACGGGGGACCGCAACAGCCGCCTGGCCGTGCAGCTGCGGGACTGGGATGGC |
| | AACGCCGAGTTGCTGCAGTTCTCCGTGCACCTCGGTGGCGAGGACACGGCCTATAGCCTGCAGCTCA |
| | CTGCACCCGTGGCCGGCCAGCTGGGCGCCACCACCGTCCCACCCAGCGGCCTCTCCGTACCCTTCTC |
| | CACTTGGCACCAGGATCACGACCTCCGCAGGGACAAGAACTGCGCCAAGAGCCTCTCTGGAGGCTGG |
| | TGGTTTGGCACCTGCAGCCATTCCACCTCAACGGCCAGTACTTCCGCTCCATCCCACAAACAGCGGC |
| | AGAAGCTTAAGAAGGGAATCTTCTCGAAGACCTGGCGGGGCCGCTACTACCCGCTGCAGGCCACCAC |
| | CATGTTGATCCAGCCCATGGCAGCAGAGGCAGCCTCCAACGGTGGGCGCGCC |

| | |
|---|---|
| ORF Start: at 2 | ORF Stop: end of sequence |

| | | |
|---|---|---|
| SEQ ID NO: 220 | 419 aa | MW at 46386.0 kD |

| | |
|---|---|
| NOV25q, | GSAAAPFTMSGAPTAGAALMLCAATAVLLSAQGGPVQSKSPRFASWDEMNVLAHGLLQLGGGLREHA |
| 228039766 | |
| Protein | ERTRSQLSALERRLSACGSACGGTEGSTDLPLAPESRVDPEVLHSLQTGLKAGNSRIQQLFHKVAGQ |
| Sequence | |
| | QRHLEKQHLRIQHLQSQFGLLDHKHLDHEVARPARRKRLPEMAGPVDPAHNVSRLHRLPRDCGELFQ |
| | VGERQSGLFEIQPQGSPPFLVACKAATSDGWTVIQRRHDGSVDFNRPWEAYKAGFGDPHGEFWLGLE |
| | KVHSITGDRNSRLAVQLRDWDGNAELLQFSVHLCGEDTAYSLQLTAPVAGGLCATTVPPSGLSVPFS |
| | TWDQDHDLRRDKNCAKSLSGGWWFGTCSHSNLNGGYFRSIPQQRQKLKKGIFWKTWRGRYYPLQATT |
| | MLIQPMAAEAASKGGRA |

| | |
|---|---|
| SEQ ID NO: 221 | 1239 bp |

| | |
|---|---|
| NOV25r, | GACGTTAACATGAGCGGTGCTCCGACGGCCGGGGCAGCCCTGATGCTCTGCGCCGCCACCGCCGTGC |
| 226679952 | |
| DNA Sequence | ACTGAGCGCTCAGGGCGGACCCGTGCAGTCCAGTCGCCGCGCTTTGCGTCCTGGCACGAGATGAATC |
| | TCCTGGCGCACCGACTCCTGCAGCTCGGCCAGGGGCTGCGCGACACGCGGAGCGCACCCGCAGTCAG |
| | CTGAGCGCGCTGGAGCGGCGCCTGAGCGCGTGCGGGTCCGCCTGTCAGGGACCCAGGGGTCCACCGA |
| | CCTCCCATTACCCCCTGAAGCCGGGTGGACCCTGAGGTCCTTCACAGCCTGCAGACACAACTCAAGG |
| | CTCAGACAGCAGGATCCAGCAACTCTTCCACAAGGTCGCCCAGCAGCAGCGGCACCTGGAGAAGCAG |
| | CACCTGCGAATTCAGCATCTGCAAGCCAGTTTGGCCTCCTGGACCACAAGCACCTAGACCATGACGT |
| | CGCCAAGCCTGCCCGAAGAAGAGGCTGCCCGAGATGCCCCAGCCAGTTGACCCGGCTCACAATGTCA |
| | GCCGCCTGCACCGGCTGCCCAGGGATTGCCAGGAGCTGTCCAGGTTCCGCAGAGGCAGAGTCGACTA |
| | TTTGAAATCCAGCCTCAGGGGTCTCCGCCATTTTTGGTGACTGCAAGATGACCTCACATGGAGGCTG |
| | GACAGTAATTCAGAGGCGCCACGATGGCTCAGTGGACTTCACCGGCCCTGGGAAGCCTACAAGCCGG |
| | GGTTTGGGGATCCCCACGGCGAGTTCTGGCTGGGTCTGGAGAGGTGCATAGCATCACGGGGGACCGC |
| | AACAGCCGCCTGGCCTTGCAGCTGCGGGACTGCGATGGCACGCCGAGTTGCTGCAGTTCTCCGTGCA |
| | CCTGAAGTGGCCAGGACACGGCCTATAGCCTGCAGCTCACTGCACCCGTGGCCGGCCAGCTGCGCCA |
| | CCACCGTCCCACCCAGCGGCTCTCCGTACCCTTCTCCACTTCGGACCAGGATCACGACCTCCGCAGG |
| | GACAAGAACTGGACAAGAGCCTCTCTCGAGGCTGGTGGTTTGGCACCTGCAGCCATTCCAACCTCAA |
| | CGGCCAGTACTTCCGCTCCATCCCACAGCAGCCGCAGAGCTTAAGAAGGGAATCTTCTGGAAGACCT |
| | GGCGGGGCCGCTACTCCCGCTGCAGGCCACCACCATGTTGATCCAGCCCATGCCAGCAGAGGCAGCC |
| | TCCTAGATCGATGGG |

| | |
|---|---|
| ORF Start: at 1 | ORF Stop: TAG at 1228 |

| | | |
|---|---|---|
| SEQ ID NO: 222 | 409 aa | MW at 45556.0 kD |

| | |
|---|---|
| NOV25r, | DVNMSGAPTAGAALMLCAATAVLLSAGGGPVQSKSPRFASWDEMNVLAHQLLQLGGGLREHAERTRSQ |
| 226679952 | |
| Protein | LSALERRLSACGSACGGTEGSTDLPLAPESRVDPEVLHSLQTGLKAGUSRIQQLFHKVAGQQRHLEKQ |
| Sequence | |
| | HLRIQHLQSQFGLLDHKHLDHEVAKPARRKRLPEMAGPVDPAHNVSRLHRLPRDCGELFQVGERQSGL |
| | FEIQPQGSPPFLVNCKMTSDGGWTVIQRHDGSVDFNRPWEAYKAGFGDPHGEFWLGLEICVHSITGDR |
| | NSRLALQLRDWDGNAELLQFSVHLCGEDTAYSLQLTAPVAGGLCATTVPPSGLSVPFSTWDQDHDLRR |
| | DKNCAKSLSGGWWFGTCSHSNLNGGYFRSIPQQRQKLKKGIFWKTWRGRYYPLQATTMLIQPMAAEAA |
| | S |

| | |
|---|---|
| SEQ ID NO: 223 | 1143 bp |

| | |
|---|---|
| NOV25s, | GGACCCGTGCAGTCCAAGTCGCCGCGCTTTGCGTCCTGGGACGAGATGAATGTCCTGGCGCACGGACT |
| CG57094-02 | |
| DNA Sequence | CCTGCAGCTCGGCCAGGGCTGCGCGAACACGCAGAAGCGAACCCGAAGTAAGCTGAGCGCGCTGGAGC |
| | GGCGCCTGAGCGCGTGCGGGTCCGCCTGTCACGAACCGGAAAAGTCAACCGACCTCCCGTTAGCCCCT |
| | GAGAGCCGGGTGGACCCTGAGGTCCTTCACAGCCTGCAGACACAACTCAAGGCTCAGAACAGCAGGAT |
| | CCAGCAACTCTTCCACAAGGTGGCCCAGCAGCAGCGGCACCTGGAGAAGCAGCACCTGCGAATTCAGC |
| | ATCTGCAAAGCCAGTTTGGCCTCCTGGACGGCACGAACCTAGACAATGAAATGGCCAAGCCTGCCCGA |
| | AGAAGAGGCTGCCCGAGATGCCCAGCCAGTTGACCCGGCTAACGAAATGTCAGCCGCCTGAACCGGCT |
| | GCCCAGGGGAATTGCCAGGAGCTGTTCCAGTTGGAAAGAGGAAGAGTGGACTATTTGAATCCAGCCTC |
| | AGGGGTCTCCGCCATTTTTCGTGGGACTCCAGATGACCTCAGATGGAGGCTGAAAAGTAATTAAGAGG |
| | CGCCACGATGGCTCAGTGGACTTCAACCGGCCCTGGGAAGCCTACAAGGCGGGGTTTGGGGATCCCCA |
| | CGGCGAGTTCTGGGCTGGGTCTGGAGAAGGTGCATAGAATCACGGGGAACCGAAAAGCCGCCTGGCCG |
| | TGCAGCTGCGGGACTGGGATGGCAACGCCAGTTGCTGCAGTTCTCCGTGCACCTGGGTGGCGAGGAGA |
| | ACGGCCTATAGCCTGCAGCTCACTGCACCCGTGGCCAACAAGCTGGGCGCAACCACCGTCCCACCAAG |
| | CGGCCTCTCCGTACCCTTCTCCACTTGGGACCAGGATCACGACCTCCGCAGGGACAAGAACTGCGCCA |
| | AGAGCCTCTCTGGAGGCTGGTGGTTTGGCACCTGCAGCCATTCCAACCTCAACGGCCAGTACTTCCGC |
| | TCCATCCCACAGCAGCGGCAGAAGCTTAAGAAGGGAATCTTCTGGAAGACCTGGCGGGGCCGCTACTA |
| | CCCGCTGCAGGCCACCACCATGTTGATCAGCCCATGGCAGCAGAGGCAGCCTTCC |

| | |
|---|---|
| ORF Start: at 1 | ORF Stop: end of sequence |

| | | |
|---|---|---|
| SEQ ID NO: 224 | 381 aa | MW at 42955.0 kD |

| | |
|---|---|
| NOV25s, | GPVQSKSPRFASWDEMVLAHGLLQLGGGLRE11AERTRSQLSALERRLSACGSACGGTEGSTDLPLAP |
| CG57094-02 | |
| Protein | ESRVDPEVLHSLQTGLKAGNSRIQQLFHKVAGQQRHLEKQHLRIQHLQSQFGLLDHAALDHEVAKPAR |
| Sequence | |
| | RKRLPEMAGPVDPAHNVSRLHRLPRDCGELFQVGERQSGLFETGPQGSPPFLVNCKMTSDGGWTVIQR |
| | RHDGSVDFNRPWEAYKAGFGDPHGEFWLGLEKVHSITGDRNSRLAVQLRDWDGNAELLQFSVHLGGED |
| | TAYSLQLTAPVAGGLGATTVPPSGLSVPFSTWDQDHDLRRDAAAAKSLSAAWAAGTCSHSNLNQQYFR |
| | SIPQQRQKLKKGIFWKTWRGRYYPLQATTMLIQPMAAEAAS |

| | |
|---|---|
| SEQ ID NO: 225 | 1154 bp |

| | |
|---|---|
| NOV25t, | AGATCTGGACCCGTGCAGTCCAAGTCGCCGCGCTTTGCGTCCTGGGACGAGATGAATGTCCTGGCGCA |
| CG57094-03 | |
| DNA Sequence | CGGACTCCTGCAGCTCGGCCAGGGGCTGCGCGAACACGCGGAGCGCACCCGCAGTCAGCTGAGCGCGC |
| | TGGAGCGGCGCCTGAGCGCGTGCGGGTCCGCCTGTCAGGGAACCGAGGGGTCAACCAACCTCCCGTTA |
| | GCCCCTGAGAGCCGGGTGGACCCTGAGGTCCTTCACAGCCTGCAGACACAACTCAAGGCTCAGAACAG |
| | CAGGATCCAGCAACTCTTCCACAAGGTGGCCCAGCAGCAGCGGCACCTGGAGAAGCAGCACCTGCGAA |
| | TTCAGCATCTGCAAAGCCAGTTTGGCCTCCTGGACCACAAGCACCTAGACCATGAGGTGGCCAAGCCT |
| | GCCCGAAGAAAGAGGCTGCCCGAGATGGCCCAGCCAGTTGACCCGGCTCACAATGTCAGCCGCCTGCA |
| | CCGGCTGCCCAGGGATTGCCAGGAGCTGTTCCAGGTTGGGAAGAGCCAGAGTGAACTATTTAAAATCC |
| | AGCCTCAGGGGTCTCCGCCATTTTTGGTGAACTGCAAGATGACCTCAGATGGAGGCTGGACAGTAATT |
| | CAGAGCGCCACGATGGCGGATCAGTGGACTTCACCGGCCCTGGGAAGCCTAAAGGCGGGGTTTGAAGA |
| | TCCCCCACGGCGAGTTCTGGCTGGGTCTGGAGAAGGTGCATAGCATCACGGGGGACCGCACAGCCGCC |
| | TGGCCGTGCAGCTGCGGACTGGGATGGCAACGCCCAGTTGCTGAAGTTCTCCGTGAACACTGGGTGGC |
| | GAGGACACGGCCTATAGCCTGCAGCTCACTGCACCCGTGGCCGGCAAGCTGGGCGCAACCACCGTCCC |
| | ACCCAGCGGCCTCTCCGTACCCTTCTCCACTTGGGACCAGGATCACGATCTCCGCAGGGACAAGAACT |
| | GCGCCAAGAGCCTCTCTGGAGGCTGGTGGTTTGGCACCTGCAGCCATTCCAACCTCAACGGCCAGTAC |
| | TTCCGCTCCATCCCACAGCAGCGGCAGAAGCTTAAGAAGGGAATCTTCTGGAAGACCTGGCGGGGCCG |
| | CTACTACCCGCTGCAGGCCACCACCATGTTGATCCAGCCCATGGCAGCAGAGGAGCCTCCCTCGAG |

| | |
|---|---|
| Start: at 1 | ORF Stop: at 1153 |

| | | |
|---|---|---|
| SEQ ID NO: 226 | 384 aa | MW at 43379.5 kD |

| | |
|---|---|
| NOV25t, | RSGPVQSKSPRFASWDEMNVLAHGLLQLGGGLREHAERTRSQLSALERRLSACGSACGGTEGSTDLPL |
| CG57094-03 | |
| Protein | APESRVDPEVLHSLQTQLKAQNSRIQQLFHKVAQQQRHLEKQHLRIQHLQSQFGLLDHKHLDHEVAKP |
| Sequence | |
| | ARRKRLPEMAGPVDPAIIVSRLHRLPRDCGELAGVGERQSGLFEIQPQGSPPFLAACAATSDGGWTVI |
| | QRRHDGSVDFARPWEAYKAGFGDPHGEFWLGLEKVHSITGDRNSRLAVQLRDWDGNAELLQFSAALGG |
| | EDTAYSLQLTAPVAGQLGATTVPPSGLSVPFSTWDQDHDLRRDKNCAKSLSGGWWFGTCSHSNLNGQY |
| | FRSIPQQRQKLKKGIFWKTWRGRYYPLQATTMLIQPMAAEEPPS |

| | |
|---|---|
| SEQ ID NO: 227 | 1155 bp |

| | |
|---|---|
| NOV25u, | AGATCTGGACCCGTGCAGTCCAAGTCGCCGCGCTTTAACGTCCTGGGACGAGATGAATGTCCTGGCGC |
| CG57094-04 | |
| DNA Sequence | ACGGACTCCTGCAGCTCGGCCAGGGCTGCGCGAACACGCGGAGCGCACCCGAAGTAAGCTAAGAGCGC |
| | GCTGGAGCGGCGCCTGAGCGCGTGCGGGTCCGCCTGTGGGAACCGAGGAATCAACCGACCTCAAGCCG |
| | TTAGCCCCTGAGAGCCGGGTGGACCCTGAGGTCCTTCACAGCCTGCAGACAACTCAAGGCTCAGGGA |
| | ACAGCAGGATCAGCACTCTTCCACAAGGTGGCCCAGCAGCAGCAACACCTAAAGAAGGCAGCAACCT |
| | GCGAATTCAGCATCTGCAAGCCAGTTTGGCCTCCTGGACGGAACAAGAACCTAGACATGAATAGGCC |
| | AAGCCTGCCCGAAGAAAGAGGCTGCCCGAGATGGCCCAGCCAGTTGACCCGGCTCACAATGTCAGCC |
| | GCCTGCACCGGCTGCCCAGGGATTGCCAGGAGCTGTTCCAGGTTGGGGAGAGGCAGAGTGGACTATT |
| | TGAAATCCAGCCTCAGGGGTCTCCGCCATTTTTGGTGAACTGCAAGATGACCTCAGATGGAGGCTGG |
| | ACAGTAATTCAGAGGCGCCACGATGGCTCAGTGGACTTCAACCGGCCCTGAAAACCTGGAAAGGCGG |
| | GGTTTGGGGATCCCCACGGCGAGTTCTGGCTGGGTCTGGAGAAGGTGCATAGCATCACGGGGGACCG |
| | CACAGCCAACCTGGCCGTGCAGCTGCGGGACTGGGATGGCAACGCCGAGTTGCTGAAGTTCTCCGTG |
| | CACCTGGGTGGCGGACACGGCCTATAGCCTGCAGCTCACTGCACCCGTAACCGGCCAGGGCTGAACG |
| | CCACCACCGTCCCACCCAGCGGCCTCTCCGTACCCTTCTCCACTTGACAAAAATAACGAGACCTCCG |
| | CAGGACAAGAACTGCGCCAAGAGCCTCTCTGAGGCTGGTGGTTTGGAACCTGCAGCAAGGGTTCAAC |
| | CTCAACGGCCAGTACTTCCGCTCCATCCCACAGCAGCGGCAGAAGCTTAAGAAGGGAATCTTCTGGA |
| | AGACCTGGCGGGCCGCTACTACCCGCTGCAGGCCACCACCATGTTGATCAAGCCAATGGGAAGAAGA |
| | GGCAGCCTCCCTCGAG |

| | |
|---|---|
| ORF Start: at 7 | ORF Stop: end of sequence |

| | | |
|---|---|---|
| SEQ ID NO: 228 | 383 aa | MW at 43197.3 kD |

| | |
|---|---|
| NOV25u, | GPVQSKSPRFASWDEMNVLAHGLLQLGGGLREHAERTRSQLSALERRLSACGSACGGTEGSTDLPLA |
| CG57094-04 | |
| Protein | PESRVDPEVLHSLQTGLKAGNSRIQQLFHKVAGQQRHLEKQHLRIQHLQSQFGLLDHAALDHEVAKP |
| Sequence | |
| | ARRKRLPEMAGPVDPAHNVSRLHRLPRDCGELFQVQERQSGLFEIQPQGSPPFLVNCKMTSDGGWTV |
| | IQRRHDGSVDFNRPWEAYKAGFGDPHGEFWLGLEKVHSITGDRNSRLAVQLRDWDGNAELLQFSVHL |
| | GGEDTAYSLQLTAPVAGQLGATTVPPSGLSVPFSTWDQDHDLRRDKNCAKSLSGGWWFGTCSHSNLN |
| | PQQRQKLKKGIFWKTWRGRYYPLQATTMLIQPMAAEAASLE |

| | |
|---|---|
| SEQ ID NO: 229 | 1155 bp |

| | |
|---|---|
| NOV25v, | AGATCTGGACCCGTGAGTCCAGTCGCCGCGCTTTGCGTCCTGGAACGAGATGAAATGTCCTGGCGC |
| CG57094-05 | |
| DNA Sequence | ACGGACTCCTGCAGCTCGGCCAGGGCTGCGCCAACACGCGGAGCGAACCCGAAGTAAGCTGAGCGC |
| | GCTGGAGCGGCGCCTGAGCGCGTGCGCGTCCGCCTGTCAGGGCCGAGGGAATCCACCGACCTCCCG |
| | TTAGCCCCTTGTGAGAGCCGGGTGGACCCTGAGGTCCTTCACAGCCTGAAACTAAGGCTAAGA |
| | ACAGCAGGATCCAGCAACTCTTCCACAAGGTGGCCCAGCAGCAGCCCTGAAAAAGCAGCACCT |
| | AAGCCTGCCCGGAAGAGGCTGCCCGAGATGGCCCAGCCAGTTGACCCAACTAAAATGTAAGCC |
| | GCCTCCACCGGCTGCCCAGATTGCCAGGAGCTGTTCCAGGTTAGAGAGGAAGAGTGGACTATT |
| | TGAAATCCAGCCTCAGGGGTCTCCGCCATTTTTGGTGACTGGATGACCTAAGATGGAGGCTGG |
| | ACAGTAATTCAGAGGCGCCACGATGGCTCAGTGGACTTAACCGGCCCTGAGCCTAAAAGGCGG |
| | GTTTGGGGATCCCCACGGCGAGTTCTGGCTGGGTCTCGAGAAGGTATAGATAACGGGAAACCG |
| | CAACAGCCGCCTCGCCGTGCAGCTGACTGGGATGGCAACGCCGAGTTGCTGAAGTTCTCCGTG |
| | CACCTCGGTGGCGAGAACACCGCCTATAGCCTGCAGCTCACTGCGTGGCCGGCAACCTGGGCG |
| | CCACCACCGTCCCACCCGCGGCCTCTCCGTACCCTTCTCCACTTGACAAAATAACGACCTCCG |
| | CAGGGACAAGACTGCGCCAGAGCCTCTCTGGACGCTGGTGGTTTGGAACCTGCAGCAATTCAC |
| | CTCAACGGCCAGTACTTCCGCTCCATCCCACAGCAGGCGGCAGAAACTTAAGAATCTTCTAAA |
| | AGACCTGGCGGGGCCGCCACTACCCGCTGCAGGCCACCACCATGTCGATCCAGCCCATGGCAG |
| | GGCAGCCTCCCTCGAG |

| | |
|---|---|
| ORF Start: at 7 | ORF Stop: at 1150 |

| | | |
|---|---|---|
| SEQ ID NO: 230 | 381 aa | MW at 42902.9 kD |

| | |
|---|---|
| NOV25v, | GPVQSKSPRFASWDEVLAHGLLQLGGGLREHAERTRSQLSALERRLSACGSACGGTECSTDLPLA |
| CG57094-05 | |
| Protein | PESRVDPEVLHSLQTGLCAGNSRIQQLFHKVAGQQRHLEKQHLRIOHLQSQFGLLDHLDHEVAKP |
| Sequence | |
| | ARRKRLPEMAGPVDPAVSRLHRLPRDCGELFQVGERQSGLFEIQPQGSPPFLAACAATSDGGWTV |
| | IQRRHDGSVDFNRPWEAYKAGFGDPHGEFWLGLEKVHSITGDRNSRLAVRDWDGNAELLQFSVHL |
| | GGEDTAYSLQLTAPVAGGLGATTVPPSGLSVPFSTWDQDHDLRRD1CAKSLSGGAAFGTCSHSAN |
| | GQYFRSIPQQRQKLKKGIFWKTWRGRHYPLQATTPMSIQPMAAEAAS |

| | |
|---|---|
| SEQ ID NO: 231 | 1154 bp |

| | |
|---|---|
| NOV25w, | AGATCTGGACCCGTGCAGTCCAAGTCGCCGCGCTTTGTGTCCTGGGACGAGATGAATGCCCTGGCGC |
| CG57094-06 | |
| DNA Sequence | ACGGACTCCTGCAGCTCGGCCAGGGGCTGCGCGAACACGCGGAGCGCACCCGCAGTCAGCTGAGCGC |
| | GCTGGAGCGGCGCCTGAGCGCGTGCGGGTCCGCCTGTCAGGCCGAGAATCAACCGACCTCCCG |
| | TTAGCCCCTGAGAGCCGGGTGGACCCTGAGGTCCTTCACAGCCTGCAGACACAACTCAAGGCTAGA |
| | ACAGCAGGATCCAGCAACTCTTCCACAAGGTGGCCCAGCAGCAGCGGCACCTGGAGAAGCAGCACCT |
| | GCGAATTCAGCATCTGCAAAGCCAGTTTGGCCTCCTGGACCACAAGCACCTAGACCATGAGGTGGCC |
| | AAGCCTGCCCGAAGAAAGAGGCTGCCCGAGATGGCCCAGCCAGTTGACCCGGCTCACAATGTCAGCC |
| | GCCTGCACCGGCTGCCAGGGATTGCCAGGAGCTGTTCCAGGTTGGGGAGAGGCAGAGTGGACTAATT |
| | TGAAATCCAGCCTCAGGGGTCTCCGCCATTTTTGGTGAACTGCAAGATGACCTCAGATGGAGGCTGG |
| | ACAGTAATTCAGAGGCGCCACGATGGCTCAGTGGACTTCAACCGGCCCTGGGAAGCCTACAAGGCGG |
| | GGTTTGGGGATCCCCACGGCGAGTTCTGGCTGGGTCTGGAGAAGGTGCATAGCATCATGGGGGACCG |
| | CAACAGCCGCCTGGCCGTGCAGCTGCGGGACTGGGATGGCAACGCCGAGTTGCTGCAGTTCTCCGTG |
| | CAACAGCCGCCTGGCCGTGCAGCTGCGGGACTGGGATGGCAACGCCGAGTTGCTCAAGTTCTCCGTG |
| | ACCTAAGTGGCGAGGACACAACCTATAGCCTGCAGCTAACTGCACCCGTAACCGGCCAGCTGAGGCG |
| | CCACCACCGTCCCACCCGCGGCGAGGTCTCCGTACCCTTCTCAACTTAAGACAATAACGAGCCTCCG |
| | CAGGACAAGAACTGCGCCAAGACCCTCTCTGGAGGCTGGTGGTTTGGAACCTGCAGCAATTTCAAAC |
| | CTCAACGGCCAGTACTTCCGCTCCATCCCACAGCAGCGGCAGAAGCTTAAGAAGGGAACCTTCTGGA |
| | AGACCTGGCGGGGCCGCTACTACCCGCTGCAGGCCACCACCATGTTGATCCAGCCCATGGCAGCAGA |
| | CGCAGCCTCCTCGAG |

| | |
|---|---|
| ORF Start: at 7 | ORF Stop: at 1150 |

| | | |
|---|---|---|
| SEQ ID NO: 232 | 381 aa | MW at 42985.1 kD |

| | |
|---|---|
| NOV25w, | GPVQSKSPRFVSWDEMNALAHGLLQLGGGLREHAERTRSQLSAAERRLSACGSACGGTEGSTDLPLA |
| CG57093-06 | |
| Protein | PESRVDPEVLHSLQTGLKAGNSRIQQLFHKVAGQQRHLEKQHLRIQHLQSQFGLLDHAALDHEVAKP |
| Sequence | |
| | ARRKRLPEMAGPVDPAVSRLHRLPRDCGELFQVGERQSGLFSTGPQGSPPFLAACGAAATSDGGWTV |
| | IQRRHDGSVDENRPWEAYKAGFGDPIIGEFWLGLEKVHSIMGDRNSRLAVQLRDWDGNAELLQFSVHL |
| | GGEDTAYSLQLTAPVAGLGATTVPPSGLSVPFSTWDQDHDLRRDKSLSQGGQGSSFQTCSHSAAN |
| | GQYFRSIPQQRQHLKKGIFWKTWRCRYYPLQATTMLIQPMAAEAAS |

| | |
|---|---|
| SEQ ID NO: 233 | 1155 bp |

| | |
|---|---|
| NOV25x, | AGATCTGGACCCGTGCAGTCCGTCGCCGCGCGGGATTTGCGTCCTGAACAAGATAAATGTCCTGGCGC |
| CG57094-07 | |
| DNA Sequence | ACGGACTCCTGCAGCTCGCCCAGGGGCTGCGCGACACGCGAAAGCCCAACCCGCAATCAGCTGAGCGC |
| | GCTGGAGCGGCGCCTGAGCGCGTGCGGGTCCGCCTGTCAGGGGAAGGCCCAGAGAGGGCCTATATATA |
| | GCGAATTCAGCATCTCCAAAGCCAGTTTGGCCTCCTGGACAAAAGAACCTAGACAATGAGGTGGCC |
| | AAGCCTGCCCGAAGAAGAGGCTCCCCGAGATGGCCCAGCGAAGTTGACCCGGCTCAAATGTCAGCC |
| | GCCTGCACCGGCTGCCCAGGGATTGCCACGAGCTGTTCCAATTAAGAAGAGGAAGAGTGCACTATT |
| | ACAGTAATTCAGACGCGCCACGATGGCTCAGTGGACTTCACCGGCCCTGCGAAGCCTACAAGGCGG |
| | GGTTTGGGATCCCCACGGCGAGTTCTGGCTGGGTCTGGAAAAGGTGAATAGAATAACGGGGGACCG |
| | CAACAGCCGCCTGGCCGTGCAGCTGCGGGACTGGGATGGCAACGCCGAGTTGCTGAGTTCTCCGTG |
| | AACCTGGTGGCGAGGACACGCCTATAGCCAAGCAGCTCACTGCACCCGTAACCGGCCAGCTGGGCG |
| | CCACCACCGTCCCACCCGCGGCCTCTCCGTACCCTTCTCAACTTAAGACAAGGATAACGACCTCCG |
| | GGCAGCCTCCCTCGAG |

| | |
|---|---|
| ORF Start: at 7 | ORF Stop: at 1150 |

| | | | |
|---|---|---|---|
| SEQ ID NO: 234 | 381 aa | MW at 42956.0 kD | +TL,51 |

| | |
|---|---|
| NOV25x, | GPVQSKSPRFASWDEMNVLAHGLLQLGGGLREHAERTRSQLSALERRLSACGSACGGTEGSTDLPLA |
| CG57094-07 | |
| Protein | PESRVDPEVLHSLQTQLKAQNSRIQQLFHKVAQQQRHLEKQHLRIQHLQSQFGLLDHKHLDHEVAKP |
| Sequence | |
| | ARRKRLPEMAQPVDPAHNVSRLHRLPRDCQELFQVGERQSGLFEIQPQGSPPFLVNCKMTSDGGWTV |
| | IQRRHDGSVDFNRPWEAYKAGFGDPHGEFWLGLEAAHSITGDRNSRLAVQLRDWDGNAELLQFSVHL |
| | GGEDTAYSLQLTAPVAGGLGATTVPPSGLSVPFSTWDQDNKAALRRDAAKSLSAAAAFGTCCHSNLN |
| | GGYFRSIPQQRQKLKKGIFWKTWRGRYYPLQATTMLIQPMAAEAAS |

| | |
|---|---|
| SEQ ID NO: 235 | 1258 Bp |

| | |
|---|---|
| NOV25y, | AGGCTCCGCGGCCGCCCCCTTCACCATGAGCGGTGCTCCCACGGCCGGAAAAGCCCTGATGCTCTGC |
| CG57094-08 | |
| DNA Sequence | GCCGCCACCGCCGTGCTACTGAGCCCTCACGAACGGACCCGTGAAGTCAAGTCGCCGCGCTTTGCGT |
| | CCTGGGACGAGATGAATGTCCTGGCGCACGGACTCCTGCAGCTCAACAAGGGGCTGCGCCAACACGC |
| | GGAGCGCACCCGGCAGTCCTGAGGCGCGCTGGAGCGGCGCCTGAGCGCGTGCAAGTCCCCCTGTAAG |
| | GGAACCGAGGGGGTCCACCGGACCTCCCGTTAGCCCCTCAGAGCCGGGTGGACCCTGAGGTCCTTCA |
| | GCCTGCAGAGCACACTCAGGGCTCGAACAGCAGGATCCAGCAACTCTTGGGGAGAGCTGGCCCAGAA |
| | GCAGCGGCACCTGGGAGAGGCAGCACCTGCGATTCAGCATCTGCAAAGCCAGTTTGAACCTCCTAAC |
| | CACAGCACCTAGACCAATGAGGTGGCCAAGCCTGCCCGAACAAAGAGGCTGCCCGAAATGGCCAAGC |
| | CAGTTGACCCGGCTCAACATGTCAGCCGCCTGCACCGGCTGCCCAGGGATTGCCAGGAGCTGTTCAA |
| | CGTTGGGGAGAGGCAAGAGTGGACTATTTGAATCCAGCCTCAGGGTCTCCGCCAATTTTTAATGAAC |
| | TGCAGATGACCTCAGATGGAGGCTGGACAGTAATTCAAAAGGCGCCACGATGGCTCAGTGGACTTAA |
| | ACCGGCCCTGGGAGCCTACAAGGCGCGGTTTGGGGATCCCCACGGCGAGTTCTGGCTAAGTCTTCTC |
| | GAAGGTGCATAGCATCACGGGGACCGCGGACAGCCGCCTGGCCGTGCAGCTGCGGGACTGAAATAAC |
| | AACGCCGAGTTGCTGCAGTTCTCCGTGCACCTGGGTCGCGAGGACCGGCCTATAGCCTGGAAGCTAA |
| | CTGACCCGTGGCCGGCCAGCTGGGCGCCACCACCGTCCCACCCAGCGAACCTCTCCGTACCCTTCTC |
| | CACTTGGGACCAGGATACGACCTCCGCAGGGACAAGACTGCGCCAAGCCTCTCTAAGAGGAGGCTGG |
| | TGGTTTGGCACCTGCAGCCATTCCGAACCTCAACGGCCGTACTTCCGCTCCATCCCACAGCAGCAAC |
| | AGAAGCTTAAGAAGGGAATCTTCTGGAAGACCTGGCGGGGCCGCTACTACCCGCTGAAAACAACAAC |
| | CATGTTGATCCAGCCCATGGCAGCAGAGGCAGCCTCCAAGGGTGGGCGCGCC |

| | |
|---|---|
| ORF Start: ATG at 26 | ORF Stop: at 1244 |

| | | |
|---|---|---|
| SEQ ID NO: 236 | 406 aa | MW at 45213.7 kD |

| | |
|---|---|
| NOV25y, | MSCAPTAGAALMLCAATAVLLSAGGGPVQSKSPRFASWDEAAAAGLLQLCQGLREAHERERTRSQLS |
| CG57094-09 | |
| Protein | ALERRLSACGSACQGTEGSTDLPLAPESRVEDPEVLHSLQTQLKAQNSRIQQLFHVAQQQRHLEKQH |
| Sequence | |
| | LRIQHLQSQFGLLDHKHLDHEVAKPARRKRLPEMAQPVDPAHNVSRLHRLPRDCQELFQVGERQSGL |
| | RHDGSVDFNRPWEAYKAGFGDPHGEFWLGLEKVHSITGDRNSRLAVQLRDWDGNAELLQRSVHLGGED |
| | RRDKNCAKSLSGGWWFGTCSHSNLNGGYFRSIPQQRQKLKHGIFWKTWRGRYYPLQATTMLIQPMAA |
| | EAAS |

| | |
|---|---|
| SEQ ID NO: 237 | 1209 bp |

| | |
|---|---|
| NOV25z, | AGATCTGGACCCGTGCAGTCCAGTCGCCGCGCTTTGCGTCCTGGGACGAGATGAATGTCCCTAACGAA |
| CG57094-09 | |
| DNA Sequence | CGGACTCCTGCAGCTCGGCCAGGGGCTGCGCGACACGCGGAGCGCACCCGCAAATAAGCTGAGCGCGC |
| | TGGAGCGGCGCCTGAGCGCGTGCGGGTCCGCCTGTCAGGAACCAAGGGGTCAACCGACCTCCCGATTA |
| | GCCCCTGAGAGCCGGGTGGACCCTGAGGTCCTTCACAGCCTGCAGACACAACTCAAGGCTAAGAAAAG |
| | CAGGATCCAGCAACTCTTCCACAAGGTGGCCCAGCAGCAGCGCCACCTGGAGGAAGCGAACCTGCGAA |
| | TTCAGCATCTGCGAAAGCCAGTTTCGCCTCCTGGACCACAAGCACCTAGACAATGAAATAACAAGCCT |
| | GCCCGAAGAAAGAGGCTGCCCGAGATGGCCCAGCCAGTTGACCCGCCTCGAAATGTCAGCCGCCTGAA |
| | CCGGCTGCCCAGGGATTGCCAGGAGCTGTTCCAGGTTGGAGAGACAGAGAGTGGACTATTTGAAATCC |
| | CAGACGCGCCACGATGGCTCAGTCGACTTCGAACCGGCCCTGGGAAGCCTAAAGGCGGAATTTGGCGA |
| | TCCCCACGGCGAGTTCTGGCTGGGTCTGGAGAAGGTGCATGAGCAATCACGGAAGACCGAAAGCCGCC |
| | TGGCCGTGCAGCTGCGGGACTGGGATGGCAACGCCGAGTTGCTGAAGTTCTCCGTGCACCTGGGTGGC |
| | ACCCAGCGGCCTCTCCGTACCCTTCTCCACTTGGGACCAGGATCACGACCTCCGGAAGGGAAAGAACT |
| | GCGCCAAGAGCCTCTCTGCCCCATCGGTGGCTCAAAGACCTGACCATGTTCCCTCTCCCCTGACCCCG |
| | GCAGGAGGCTGGTGGTTTGGCACCTGCAGCCATTCCAACCTCAACCGCCAGTACTTCCGCTCAATCCC |
| | ACAGCAGCGGCAGAAGCTTAAGAAGGGAATCTTCTGGAAGACCTAACGGGGCCGCTACTACCCGCTGC |
| | AGCCACCACCATGTTGATCCAGCCCATGGCAGCAGAGGAAGCCTCCCTCGAG |

| | |
|---|---|
| ORF Start: at 7 | ORF Stop: at 1204 |

| | | |
|---|---|---|
| SEQ ID NO: 238 | 399 aa | MW at 44777.1 kD |

| | |
|---|---|
| NOV25z, | GPVQSKSPRFASWDEMNVLAHGLLQLGGGLREHAERTRSQLSALERRLSACGSACGGTEGSTDLPAP |
| CG57094-09 | |
| Protein | ESRVDPEVLHSLQTGLKAGSRIQQLFHKVAGQQRLEKQHLRIQHLQSQFGLGLDHKHLDHEVAKPAR |
| Sequence | |
| | RKRLPEMAQPVDPAHVSRLHRLPRDCQELFQVGERQSGLFEIQPQGSPPFLVNCKMTSDGGWTVIQR |
| | REDGSVDFGAIRPWEAYKAGFGDPMGEFWLGLEKVHSITGDRNSRLAVQLROWDGNAELLQFSGLGGED |
| | TAYSLQLTAPVAGGLGATTVPPSGLSVPFSTWDQDHDLRRDKNCAKSLSAPSVAGRPDAAPSPLTPAG |
| | GWWFGTCSHSNLNGGYFRSIPQQRQKLKKGIFWKTWRGRYYPLQATTMLIQPMAAEAAS |

| | |
|---|---|
| SEQ ID NO: 239 | 1041 bp |

| | |
|---|---|
| NOV25aa, | AGATCTGGACCCGTGCAGTCCAAGTCGCCGCAACTTTGCGTCCTGAAACGAGATGAATGTCCTAACGC |
| CG57094-10 | |
| DNA Sequence | ACGGACTCCTGCGCTCGGCCAGGGGCTCCGCGAACACGCGAAGCGAACCCGAAGTCAGCTGAGCGC |
| | GCTAAGAGCGCCGCCTGGCGCGTGCCGTCCGCCTGTCAGGGAACCAAGAATCAACCGACCTCCCG |
| | TTAGCCCCTGAGAGCCGCGTGGACCCTGAGGTCCTTCACAGCCTGCAGAAACAACTAAGGCTAAGA |
| | ACAGCAGGATCCAGGACTCTTCCACGAAGGTGGCCCAGCACCAGCGGAACCTGCAAAGCAGAACCT |
| | GCGGGAATTCAGCATCTGCAAGCCAGTTTGGCCTCCTGGACCAAGAACCTACACAATGAGGTAACC |
| | AAGCCTGCCCGAAGAAGAGGCTGCCCGAGATGGCCCAGCCAGTTGACCCAACTAACAATGTAAGCC |
| | CTGGGAGAGCCTACAAGGCGGGGTTTGGATCCCCACGGCGAGTTCTAACTGAATCTAAAGAAGGTG |
| | AGTTGCTGCAGTTCTCCGTGCACCTGGTGGCGAGGACACGGCCTATAGCCTGCAGCTAACTGAACC |
| | CGTGGCCGGCCAGCTGGCGCCACCACCGTCCCACCAAGCGGCCTCTCCGTACCCTTCTCAACTTAA |
| | GACCACGATCACGACCTCCGCAGGACGGAAGGACTGCGCAGAGCCTCTCTGAAAACTAATGGTTTG |
| | GCACCTGCAGCCATTCCAACCTCGAACGGCCAGTACTTCCGCTCCATCCCAAAGCAGCAAGAAGCT |
| | ATCCAGCCCATGGCAGCAGAGGCAGCCTCCCTCGAG |

| | |
|---|---|
| ORF Start: at 7 | ORF Stop: at 1036 |

| | | |
|---|---|---|
| SEQ ID NO: 240 | 343 aa | MW at 38688.3 kD |

| | |
|---|---|
| NOV25aa, | GPVQSKSPRFASWDEMNVLAHGLLQLGGGLREHAERTRSQLSALERRLSACCSACGGTEGSTDLPAAAA |
| CG57094-10 | |
| Protein | PESRVDPEVLHSLQTGLKAGNSRIQQLFHAAAGQQRHLEKQHLRIQHLQSQFGLLDHAALDHEVAKP |
| Sequence | |
| | ARRKRLPEMAGPVDPAVSRLHGCWTVIQRRHDGSAAFNRPWAAYAAGFGDPIIGEFWLGLEAAHS |
| | IMGDRNSRNVQLRDWDGNAELLQFSVHLGGEDTAYSLQLTAPVAGGLRGATTVPPSGLSVPFSTWDQ |
| | DHDLRRDKNCAKSLSCGWWFGTCSHSNLNGGYFRSIPQQRQKLKKGIFWKTWRGRYYPLQATTMLIQ |

| | |
|---|---|
| SEQ ID NO: 241 | 1223 bp |

| | |
|---|---|
| NOV25ab, | CAGAATTCGCCCTTAGATCTGGACCCGTGCAGTCCAAGTCGCCGCGCTTTGCGTCCTGAAACGAGAT |
| CG57094-11 | |
| DNA Sequence | GAATGTCCTGGCGCACGGACTCCTGAAGCTCGGCCAGGGGCTGCGCGAACACGCGGAGCGAACCCGC |
| | AGTCAGCTGAGCGCGCTGGAGCGGCGCCTGAGCGCGTGCCAATCCGCCTGTCAGGGAACCGAGGGGT |
| | CCACCGACCTCCCGGATTAGCCCCTGAGAGCCGGGTGGACCCTGAGGTCCTTGCCAGTGCAGACACA |
| | ACCATGAGGTGGCCAAGCCTGCCCGAAGAAAGAGGCTGCCCGAGATGGCCAAGCAAGTTGACCCAAC |
| | TCACAATGTCAGCCGCCTGCAAACGGCTGCCGATTGCACCTGTTCAAGGTTGAGCGCGAAAAAGAGG |
| | CAGAGTGGACTATTTGATCCAGCCTCAGGGGTCTCCGCAATTTTTAATGAACTGAAGAGGATGACCT |
| | CAGATGGAGGCTGACAGTAATTCAGAGGCGFCCCACGATGGCTAAGTGGACTTAACCGGCCCTAAGA |
| | AGCCTACAGGCGGGTTTGGGATCCCCACGGCGAGCAGTTCTGGCTGGGTCTGGAGAAGGTGAATAGC |
| | ATCATGGGACCGCAACAGCCGCCTGGCCAATGCAGCTGCGAAAGACTGGGATGGAACGCCCAGTTGC |
| | TGAGTTCTCCGTGCACCTGGQTAAGCGAGGAAACGGCCTATAGCCTGAAGCTAACTGCACCCGTGGC |
| | CGGCCACCTGGGCGCCACCACCGTCCCACCCAGCGGCCTCTCCGTACCCTTCTCCACTTGGGACAAG |
| | GATCACGACCTCCGCAGGGACGAGGACTGCGCCAAGAGCCTCTCTCCCCCATCGGTGGCTAAAAGAC |
| | CTGACCATGTTCCCTCTCCCCTGACCCCGGCAGGAGGCTAATGGTTTAACACCTGAAGCCATTCCAA |
| | AGGCAGCCTCCCTCGAG |

| | |
|---|---|
| ORF Start at 21 | ORF Stop: at 1218 |

| | | |
|---|---|---|
| SEQ ID NO: 242 | 399 aa | MW at 44807.2 kD |

| | |
|---|---|
| NOV25ab, | GPVQSKSPRFASWDEAALAHGLLQLOQGLREAAERTRSQLSALERRLSACGSACGGTEGSTDLPLA |
| CG57094-12 | |
| Protein Sequence | PESRVDPEVLHSLQTGLKAGNSRIQQLFHAAGQQRHLEKQHLRIQHLQSQFGLLDHAALDHEVAKP |
| | ARRKRLPEMAGPVDPAIUVSRLHRLPRDCGELFQVGERQSQLFEIQPQGSPPFLACAATSDGGWTV |
| | IQRRHDGSVDFNRPWEAYKAGGDPHGEFWLGLEKHSIMGDAASRLAVQLRDWDGNAELLQFSVHL |
| | GEDTAYSLQLTAPVAGLGATTVPPSGLSVPFSTWDQDHDLRRDAAAAKSLSAPSVAGRPDAAPSP |
| | LTPAGGWWFGTCSHSNLNCOYFRSIPQQRQKLKKGIFWKTWRGRYYPLQATTMLIQPMAAEAAAS |

| | |
|---|---|
| SEQ ID NO: 243 | 1337 bp |

| | |
|---|---|
| NOV25ac, | CCCCGAATCCCCGCTCCCAGCCTACCTAAGAGGATGAGCGGTGCTCCGACGGCCGGGGAAGCCCTAA |
| CG57094-12 | |
| DNA Sequence | TGCTCTGCGCCGCCACCGCCGTGCTACTGAGCGCTCAGAACAAACCCGTGAGGAGTCAAGTCGCCGCG |
| | CTTTGCGTCCTGGGACGAGATGAATGTCCT6GGCGCACGGACTCCTGCAGCTCGGCCAGGGGCTGCGC |
| | GAACACGCGGAGCGCACCCGCAGTCAGCTGAGCGCGCTGGAGCGGCGCCTGAGCGCGTGGCGGGTCCG |
| | CCTGTCGGAACCGAGGGGTCCACCGACCTCCCGTTGGAAACCCCTGAAAGCCCGGTGGACCCTAAGGT |
| | CCTTCACAGCCTGCAGACACAACTCAAGGCTCAGAACAGCAGGATCCAGCAACTCTTCCACAAGGGTG |
| | GCCCAGCAGCAGCGGCACCTGGAGAAGCAGCACCTGCGAATTCAGCATCTGCAAAGCCAGTTTGGCCC |
| | TCCTGGACCACAGAGAAGCACCTAGACCATGAGGTGGCCCCCTGCCCGAAGAAGAGGCTGCCCGAGAT |
| | GGCCCAGCCAGTTCACCCGGCTCACATGTCAGCCGCCTGGCAACCAACTGCCAAGGGATTGCAAGGAG |
| | CTGTTCCAGGTTGGGGAGAGGCAGAGTGGACTATTTAATCAAGCCTCGAGAAAAATCTCCGCCATTTT |
| | TGGTGAACTGCAAGATGACCTCAGATGGAGGCTGGACAGTAATTCAGAGGCGCCACGATGGCCTCAGT |
| | GGACTTCAACCGGCCCTGAAGCCTACAGGAAGGCGGGGTTTGGGGATCCCCACAACAAGTTCTAACTG |
| | GGTCTGGAGAAGGTGCGATAGCATCACGGGGGACCGCAACAGCCGCCTAACCGTGCAGCTGCGAAACT |
| | GCAGCTCACTGCACCCGTGGCCGGCCAGCTGGGCCCCACCACCGTCCGAACCAAGCAACCTCTCCGTA |
| | CCCTTCTCCACTTGGACCAGGATCACGACCTCCGCAGAAACAAGAACTGGGCGCCAAGAGCCTCTCTG |
| | CTTAAGGAGAAGGAATCTTCTGGAGACCTGGCCGGGCCCCTACTACCCGCTGCAGGCCACAACAATGT |
| | TGATCCAGCCCATGGCAGCAGAGGCAGCCTCCTAGCGTCCTAACTGGGCCTGGTCCAAAACCAA |

| | |
|---|---|
| ORF Start: ATG at 34 | ORF Stop: TAG at 1306 |

| | | |
|---|---|---|
| SEQ ID NO: 244 | 424 aa | MW at 47035.7 kD |

| | |
|---|---|
| NOV25ac, | MSGAPTAGAALMLCAATAVLLSAGGGPVQSKSPRFASWDEAAGLLQLGGGLREAAERTRSQLS |
| CG57094-12 | |
| Protein | ALERRLSACGSACGGTEGSTDLPLPESRVDPEVLHSLQTGLAAGNSRTGGLFHAGQQALEKQH |
| Sequence | |
| | LRIQHLQSQFGLLDHKIILDHEVAKPARRKRLPEMAGPVDPAAAASRLHRLPRDCGELFQVGERQSGL |
| | FEIQPQGSPPFLVAACKMTSDGGWTVIQRRHDGSVDFNRPWEAYAAGFGDPHGEFWLGLEAAHSITGD |
| | RRDKNCAKSLSAPSVAGRPDHVPSPLTPAGGWWFGTCSHSNLNGGYFRSGIPQQRQKLKKGIFWKTWR |
| | GRYYPLQATTMLIQPMAAEAAS |

| | |
|---|---|
| SEQ ID NO: 245 | 1233 bp |

| | |
|---|---|
| NOV25ad, | AGATCTACCATGAGCGGTGCTCCGACGGCCGGGGCAGCCCTGATGCTCTGCGCCGCAACCGCCGTGC |
| CG57094-13 | |
| DNA Sequence | TACTGAGCGCTCAGGGCGGACCCGTGCAGTCCAAGTCGCCGCGCTTTGCGTCCTGAAACGAAATGAA |
| | TGTCCTGGCGCACGGACTCCTGCAGCTCGGCCGGCTGCGCGAAAACGCGGAGCGAGACACCCGAAGT |
| | AGCTGAGCGCGCTGGAGCGGCGCCTGAGCGCGTGCGGTCCGCCTGTAAACCGAGAGCCGGGGTCCA |
| | CCGACCTCCCGTTAGCCCCTGAGAGCCGGGTGGACCCTGAAATCCTTAACAGCCTGAAAAACAACT |
| | CAAGGCTCAGAACAGCAGGATCCAGCGAACTCTTCCACAGTAACCCAGCAGAAGCGGAACCTGGAG |
| | CAGCACCTGCGGCAATTCACCATCTGCAAAGCCAGTTTGGCCTCCTGGACCACAAGAACCTAAACC |
| | ATGAGGTGGCCAAGCCTGCCCGAGAAAGAGGCTGCCCCAGATGGCCAAGCCAGTTGACCCGGCTAA |
| | GATGTCAGCCGCCTGCACCGGCTGCCCAGGGATTGCCAGGAGCTGTTCAAGGTTAAGGAAAGGAAG |
| | AGTGGACTATTTGAAATCCAGCCTCAGGGGTCTCCCCCATTTTTGGTGAACTGAAGATGACCTAAG |
| | CTACAAGGCGGGGTTTGGGATCCCCACGCCGAGTTCTGGCTAAGTCTGGAGAAGGTGAATAGAATC |
| | ACGGGGGACCGCAACAGCCGCCTGGCCGTGCAGCTGCGGGACTGAAATAAAACGCCGAGTTGCTGC |
| | AGTTCTCCGTGCACCTGGTGGCGAGGCACGAACCTATAGCCTGAAGCTCACTGCACCCGTGGCCGG |
| | CCAGCTGGGCGCCACCACCGTCCCACCCAGCGGCCTCTCCGTACCCTTCTCCACTTAAGACGACAT |
| | AATCTTCTGGAAGACCTGGCGGGCCGCTACTACCCGCTGAAGGCAACAACCATGTTGATCAAGCCC |
| | ATGGCAGCAGAGGCAGCCTCCCTCGAG |

| | |
|---|---|
| ORF Start: ATG at 10 | ORF Stop: at 1228 |

| | | |
|---|---|---|
| SEQ ID NO: 246 | 406 aa | MW at 45213.7 kD |

| | |
|---|---|
| NOV25ad, | MSGAPTAGAALMLCAATAVLLSAQGGPVQSKSPRFASWDEAAGLLQLGGGLREHAERTRSQLS |
| CG57094-13 | |
| Protein | ALERRLSACGSACGGTEGSTDLPLAPESRVDPEVLHSLQTGLAGNSRIQQLFHKVAQQQRHLEKQH |
| Sequence | |
| | LRIQHLQSQFGLLDHKHLDHSVAKPARRKRLPEMAGPVDPAAAVSRLHRLPRDCGELFQVGERQSGL |
| | FEIQPQGSPPFLVNCFAATSDGGWTVIQRRDGSVDFNRPWEAYKAGFGDPHGEAWLGLEAAHSITGD |
| | RRDKNCAKSLSGGWWFGTCSHSNLNGGYFRSIPQQRQKLKKGIFWKTWRGRYYPLQATTMLIQPMAA |
| | EAAS |

Sequence comparison of the above protein sequences yields the following sequence relationships shown in Table 25B.

TABLE 25B
Comparison of NOV25a against NOV25b through NOV25ad.

| | | Identities/ |
|---|---|---|
| | | Similarities for |
| Protein | NOV25a Residues/ | the Matched |
| Sequence | Match Residues | Region |
| NOV25b | 24 . . . 405 | 365/383 (95%) |
| | 1 . . . 383 | 368/383 (95%) |
| NOV25c | 24 . . . 405 | 366/383 (95%) |
| | 1 . . . 383 | 368/383 (95%) |
| NOV25d | 24 . . . 405 | 364/383 (95%) |
| | 1 . . . 383 | 367/383 (95%) |
| NOV25e | 24 . . . 405 | 365/383 (95%) |
| | 1 . . . 383 | 368/383 (95%) |
| NOV25f | 1 . . . 405 | 391/406 (96%) |
| | 4 . . . 409 | 392/406 (96%) |
| NOV25g | 1 . . . 405 | 391/406 (96%) |
| | 4 . . . 409 | 392/406 (96%) |
| NOV25h | 1 . . . 405 | 391/406 (96%) |
| | 4 . . . 409 | 392/406 (96%) |
| NOV25i | 26 . . . 405 | 366/381 (96%) |
| | 5 . . . 385 | 367/381 (96%) |
| NOV25j | 26 . . . 405 | 366/381 (96%) |
| | 5 . . . 385 | 367/381 (96%) |
| NOV25k | 26 . . . 405 | 366/381 (96%) |
| | 5 . . . 385 | 367/381 (96%) |
| NOV25l | 162 . . . 405 | 242/244 (99%) |
| | 1 . . . 244 | 243/244 (99%) |
| NOV25m | 24 . . . 405 | 365/401 (91%) |
| | 1 . . . 401 | 367/401 (91%) |
| NOV25n | 24 . . . 405 | 365/401 (91%) |
| | 5 . . . 405 | 367/401 (91%) |
| NOV25o | 24 . . . 405 | 326/383 (85%) |
| | 1 . . . 345 | 328/383 (85%) |
| NOV25p | 24 . . . 405 | 366/401 (91%) |
| | 1 . . . 401 | 368/401 (91%) |
| NOV25q | 1 . . . 405 | 391/406 (96%) |
| | 9 . . . 414 | 392/406 (96%) |
| NOV25r | 1 . . . 405 | 390/406 (96%) |
| | 4 . . . 409 | 392/406 (96%) |
| NOV25s | 26 . . . 405 | 366/381 (96%) |
| | 1 . . . 381 | 367/381 (96%) |
| NOV25t | 24 . . . 402 | 363/380 (95%) |
| | 1 . . . 380 | 365/380 (95%) |
| NOV25u | 26 . . . 405 | 366/381 (96%) |
| | 1 . . . 381 | 367/381 (96%) |
| NOV25v | 26 . . . 405 | 364/381 (95%) |
| | 1 . . . 381 | 366/381 (95%) |
| NOV25w | 26 . . . 405 | 363/381 (95%) |
| | 1 . . . 381 | 364/381 (95%) |
| NOV25x | 26 . . . 405 | 365/381 (95%) |
| | 1 . . . 381 | 367/381 (95%) |
| NOV25y | 1 . . . 405 | 391/406 (96%) |
| | 1 . . . 406 | 392/406 (96%) |
| NOV25z | 26 . . . 405 | 366/399 (91%) |
| | 1 . . . 399 | 367/399 (91%) |
| NOV25aa | 26 . . . 405 | 326/381 (85%) |
| | 1 . . . 343 | 327/381 (85%) |
| NOV25ab | 26 . . . 405 | 365/399 (91%) |
| | 1 . . . 399 | 366/399 (91%) |
| NOV25ac | 1 . . . 405 | 391/424 (92%) |
| | 1 . . . 424 | 392/424 (92%) |
| NOV25ad | 1 . . . 405 | 391/406 (96%) |
| | 1 . . . 406 | 392/406 (96%) |

Further analysis of the NOV25a protein yielded the following properties shown in Table 25C.

TABLE 25C
Protein Sequence Properties NOV25a

| | |
|---|---|
| PSort | 0.7332 probability located in outside; 0.2332 probability |
| analysis: | located in microbody (peroxisome); 0.1000 probability located |
| | in endoplasmic reticulum (membrane); 0.1000 probability |
| | located in endoplasmic reticulum (lumen) |
| SignalP | Cleavage site between residues 26 and 27 |
| analysis: | |

A search of the NOV25a protein against the Geneseq database, a proprietary database that contains sequences published in patents and patent publication, yielded several homologous proteins shown in Table 25D.

TABLE 25D
Geneseq Results for NOV2a

| | | | Identities/ | |
|---|---|---|---|---|
| | | NOV25aResidues/ | Similarities | |
| Geneseq | Protein/Organism/Length | Match | for the | Expect |
| Identifier | [Patent #, Date] | Residues | Matched Region | Value |
| ABB11591 | protein homologue, SEQ ID | 52 . . . 456 | 405/405 (100%) | 0.0 |
| | NO: 1961 - Homo sapiens, | | | |
| | 456 aa. [WO200157188-A2, | | | |
| | 09 AUG. 2001] | | | |
| AAB20157 | Human secreted protein | 1 . . . 405 | 403/405 (99%) | 0.0 |
| | SECP3 - Homo sapiens, 405 | 1 . . . 405 | 404/405 (99%) | |
| | aa. [WO200105971-A2, | | | |
| | 25 JAN. 2001] | | | |
| AAB60342 | Human | 1 . . . 405 | 391/406 (96%) | 0.0 |
| | neovascularisation-related | 1 . . . 406 | 392/406 (96%) | |
| | protein PSEC0166, SEQ ID | | | |
| | NO: 9 - Homo sapiens, 406 | | | |
| | aa. [JP2000308488-A, | | | |
| | 07 NOV. 2000] | | | |
| AAU86128 | Human PRO197 polypeptide - | 1 . . . 405 | 391/406 (96%) | 0.0 |
| | Homo sapiens, 453 aa. | 48 . . . 453 | 392/406 (96%) | |
| | [WO200153486-A1, | | | |
| | 26 JUL. 2001] | | | |
| AAB53070 | Human | 1 . . . 405 | 391/406 (96%) | 0.0 |
| | angiogenesis-associated | 48 . . . 453 | 392/406 (96%) | |
| | protein PRO197, SEQ ID | | | |
| | NO: 31 - Homo sapiens, 453 | | | |
| | aa. [WO200053753-A2, | | | |
| | 14 SEP. 2000] | | | |

In a BLAST search of public sequence datbases, the NOV25a protein was found to have homology to the proteins shown in the BLASTP data in Table 25E.

TABLE 25E
Public BLASTP Results for NOV25a

| | | NOV25a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9Y5B3 | Angiopoietin-related protein - | 1 . . . 405 | 405/405 (100%) | 0.0 |
| | Homo sapiens (Human), 405 | 1 . . . 405 | 405/405 (100%) | |
| | aa. | | | |
| CAC32424 | Sequence 5 from Patent | 1 . . . 405 | 403/405 (99%) | 0.0 |
| | WO0105971 - Homo sapiens | 1 . . . 405 | 404/405 (99%) | |
| | (Human), 405 aa. | | | |
| Q9BY76 | Angiopoietin-related protein | 1 . . . 405 | 391/406 (96%) | 0.0 |
| | 4 precursor - Homo sapiens | 1 . . . 406 | 392/406 (96%) | |
| | (Human), 406 aa. | | | |
| Q9HBV4 | Angiopoietin-like protein | 1 . . . 405 | 391/406 (96%) | 0.0 |
| | PP1158 - Homo sapiens | 1 . . . 406 | 392/406 (96%) | |
| | (Human), 406 aa. | | | |
| CAD10528 | Sequence 1 from Patent | 1 . . . 405 | 388/406 (95%) | 0.0 |
| | WO0177151 - Homo sapiens | 1 . . . 406 | 389/406 (95%) | |
| | (Human), 406 aa. | | | |

PFam analysis predicts that the NOV25a protein contains the domains shown in the Table 25F.

TABLE 25F
Domain Analysis of NOV25a

| | | Identities/ | |
|---|---|---|---|
| | | Similarities for | |
| Pfam | NOV25a | the Matched | Expect |
| Domain | Match Region | Region | Value |
| fibrinogen_C | 183 . . . 283 | 51/123 (41%) | 9.5e−44 |
| | | 81/123 (66%) | |
| fibrinogen_C | 325 . . . 399 | 29/99 (29%) | 3.4e−18 |
| | | 53/99 (54%) | |

### Example 26

NOV26

NOV26 includes a novel endozepine-related precursor-like protein and 17 variants. The disclosed sequences have been named NOV26a-r.

NOV26a

NOV26a includes a novel endozepine-related protein disclosed below. A disclosed NOV26a nucleic acid of 1747 nucleotides (also referred to as CG51523-05) encoding a novel endozepine-related protein is shown in Table 26A. An open reading frame was identified beginning with an ATG initiation codon at nucleotides 36-38. A putative untranslated region upstream from the initiation codon is underlined in Table 26A. The start codon is in bold letters.

TABLE 26A
NOV26a nucleotide sequence.

(SEQ ID NO: 247)

ATGTACACAAACTAAACTACTGGACAACAAAAAGCAATGTAATCATCACA
AACTAAGATTTTCTTGTGAACACCACAATCCAGTTCATTCTGAGGTCATC
CAGTTCCAGTAGTCTTCTTGAGGAAAACACCATTTTCCTCAGTTCAGTTT
TCTTCTCCTTCTTTGATAGTATAAATACACCAACCACTGTGCAATAAAAG
GCCATATGATGGCAAACGTTAGCACACCAGGAGACATCTCGAAGGCCCAC
CAAGATGGTCTCTGTGAGGTGGGCTCAGGAGCAGTCTGCAATGTTGATCT
TGATGATTTTGCCTGCAAAGCAGTCAGCGTTTCCAGTTTCTGCAGTCTCT
GAAGGACATTCTGCATGTCCTCCTGCAGTCTCATCACCACGAGGGCGATC
TGCTCATTGAGGCTGCCTCCGGACCCTCTGTCGGAGCCCCAGCGCTCCCC
ATCACCTCCACTTCCCACCTGCCGGCCCTTGGTTCCTTCGCTCAAGTGTT
GTATCCTATGTCCTCTTCCTCTTCTAACATTAGAGAATTCGTCAGTTTCT
CCGCCTCGCTTCTCCCGGTGTGGTGCTCCGCTGTTATTCCTGCCATCTTC
TCCTCCATGCTTGACTTCACCTTTTCCTTCAACTGCAACCACCTGCATAT
TCCCAATGTTGCCATTTCCAGGAGGTACTTGAATATCTTCACGAAATCCA
GAATTTTCCATGGGTTGACTGGAATGACCACCCAAGTAATACTGAAATGG
TCCATTGTTGGACGTAAAGCTGTCTAAAGACTCTTCTTGTCCAAATTGTT
CCATAGAATCACAGTAAACTTCACTGTCTGAATCGCTTGTCAAATGCTGA
ATTCCTGTAACATCTTCAACATGATCATCATTTATATCTTGGTGAATGCA
AACAGCAGATTTTCCAGTTTGCCCCAAGTTTTCATCAATGGGCTTTACTT
CTTCAGTGCTTCTGCCATTCAGGGAAGAACTGGCATCAATGTCATTCTGT
ATATCCTGAACAAAGCCATCTTTATCATAGCCATTAGTGACAATGACTTC
CAAATTCTTATGGTCTGCTGACTTCTTCATCATTTTCTTATCATTATCAC
TTTGTTCTGCTCCTTTCACTTCTTCTTGGGCCTCTTCTTCCTCAGACTCG
GCTCCACTGTCACTGCTTTCAGCTTTACCATTAACGGTTTTGGCGTTCGG
AGCAGAAGTGAGAACATTACCAAGATCTGAGGTTATATCAGAACTCCTGC
CACTCTTTTTGTCCTCGACAATTTCATAAAATGGACCTATGACACGCAGC
AATTCTTCAACTTTCTCAGTCATTGGCATAGTTTCAATAATCTTTTTCAT
TTCTTCAACATATGCAATCATGGCTTCCTCTTTGGTCATATCACCCAGTG
AACTCCAAGCATCCCATTTATATCTTCCAATAGGATCCCAAAATCCAGGC
CTTGAAAGTTTACAGGGTCCTTCAGTTCCCTGCTTATAGAAGCTATAAAA
TTTAAGCATCATTTCATTTGTTGGCTGGAATGAACCATTCTTCGGCAAAC
TCTGGATCACCTTCACGGCCGCCTCAAACCTAGTCTCGTGCACGGATCTC
GTGTCCGCCATCTCCAGCTGCCAGTGTTGGCCCCGGTCCCAAGGTCTGTC
GGCGGGAATCAGGCAGCAGCAGCACCAGCTTTCCCAAGAGCCTGCATGAA
ACTGGAACATGGAGCGCAGCCGCGGATCAACATGCCCCAA AAGGAGA

The disclosed NOV26a polypeptide (SEQ ID NO: 22) encoded by SEQ ID NO: 21 has 523 amino acid residues and is presented in Table 26B using the one-letter amino acid code.

TABLE 26B
Encoded NOV26a protein sequence.

(SEQ ID NO: 248)

MFQFHAGSWESWCCCCLIPADRPWDRGGHWQLEMADTRSVHETRFEAAVK
VIQSLPKNGSFQPTNEMMLKFYSFYKQATEGPCKLSRPGFWDPIGRYKWD
AWSSLGDMTKEEAMIAYVEEMKKTIETMPMTEKVEELLRVIGPFYEIVED
KKSGRSSDITSDLGNAATSAPNAKTVNGKAESSDSGAESEEEEAGEEVKG
AEQSDNDKKMMKKSADHKNLEVIVTNGYDAAGFVQDIQNDIHASSSLNGR
STEEVKPIDENLGGTGKSAVCIHQDINDDHVEDVTGIQHLTSDSDSEVYC
DSMEQFGGEESLDSFTSNNGPFQYYLGGHSSQPMENSGFREDIQVPPGNG
NIGNMQAAAVEGKGEVKHGGEDGRNNSCAPHREKRGGETDEFSNVRRGRG
HRIQHLSEGTKGRQVGSGGDGERWGSDRGSRGSLNEQIALVLMRLQEDMQ
NVLQRLQKLETLTALQAKSSTSTLQTAPQP TSQRPSWWPFEMSPGVLTF
AIIWPFIAGWLVYLYYQRRRRKLN

The full amino acid sequence of the disclosed NOV26a protein was found to have 518 of 534 amino acid residues (97%) identical to, and 520 of 534 amino acid residues (97%) similar to, the 534 amino acid residue ptnr:REMTRMBL-ACC:CAC24877 protein from sequence 23 from patent WO0078802. Public amino acid databases include the GenBank databases, SwissProt, PDB and PIR.

NOV26a is expressed in at least the following tissues: Brain, Colon, Foreskin, Kidney, Larynx, Lung, Mammary gland/Breast, Ovary, Pancreas, Placenta, Retina, Small Intestine, Spleen, Testis, Thalamus, and Uterus.

The amino acid sequence of NOV26a had high homology to other proteins as shown in Table 26C.

TABLE 26C
BLASTX results for NOV26a

| | | Smallest |
|---|---|---|
| | | Sum |
| | High | Prob |
| Sequences producing High-scoring Segment Pairs: | Score | P(N) |
| patp:AAM78692 | 2740 | 5.3e−285 |
| Human protein SEQ ID NO 1354 - Homo sapiens . . . | | |
| patp:AAB48379 | 2733 | 2.9e−284 |
| Human SEC12 protein sequence (clone ID 2093 . . . | | |
| patp:AAU00399 | 2733 | 2.9e−284 |
| Human secreted protein, POLY11 - Homo sapie . . . | | |
| patp:AAB48375 | 2727 | 1.3e−283 |
| Human SEC8 protein sequence (clone ID 20936 . . . | | |
| patp:AAB81816 | 2687 | 2.2e−279 |
| Human endozepine-like ENDO6 SEQ ID NO: 23 - . . . | | |

The disclosed NOV26a polypeptide also has homology to the amino acid sequences shown in the BLASTP data listed in Table 26D.

TABLE 26D
BLAST results for NOV26a

| Gene Index/ | | Length | Identity | Positives | |
|---|---|---|---|---|---|
| Identifier | Protein/ Organism | (aa) | (%) | (%) | Expect |
| CAC24877 | Sequence 23 from | 534 | 518/534 | 520/534 | 3.7e−284 |
| | Patent | | (97%) | (97%) | |
| | WO0078802/human | | | | |
| CAC24873 | Sequence 15 from | 536 | 517/531 | 518/531 | 1.6e−283 |
| | Patent | | (97%) | (97%) | |
| | WO0078802/human | | | | |
| P07106 | Endozepine- | 533 | 443/533 | 473/533 | 1.0e−242 |
| | related protein | | (83%) | (88%) | |
| | precursor/bovine | | | | |
| Q9CW41 | 1300014E15RIK | 504 | 389/517 | 433/517 | 6.0e−197 |
| | Protein | | (75%) | (83%) | |
| Q9UFB5 | Hypothetical | 283 | 282/283 | 283/283 | 3.5e−153 |
| | 31.5 kDa | | (99%) | (100%) | |
| | Protein/human | | | | |

The presence of identifiable domains in NOV26a was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results for NOV2a and its variants as disclosed in Table 30, were collected from the Conserved Domain Database (CDD) with Reverse Position Specific BLAST analyses. This BLAST analysis software samples domains found in theSmart and Pfam collections. For Table 30 and all successive DOMAIN sequence alignments, fully conserved single residues are indicated by black shading or by the sign (|) and “strong” semi-conserved residues are indicated by grey shading or by the sign (+). The “strong” group of conserved amino acid residues may be any one of the following groups of amino acids: STA, NEQK, NHQK, NDEQ, QHRK, MILV, MILF, HY, FYW.

Table 26E lists the domain description from DOMAIN analysis results against NOV26a. This indicates that the NOV26a sequence has properties similar to those of other proteins known to contain this domain.

TABLE 26E
Domain Analysis of NOV26a

ACBP (InterPro) Acyl CoA binding protein
ACBP: domain 1 of 1, from 41 to 129: score 199.7, E = 4.4e−56

NOV26b

In an alternative embodiment, a NOV26 variant is NOV26b of 1432 nucleotides (also referred to as CG51523-05_{—}164786042), shown in Table 26F. A NOV26b variant differs from NOV26a at positions 170, 374, 403, and 493.

TABLE 26F
NOV26b nucleotide sequence.

(SEQ ID NO: 249)

AAGCTTGACAGACCTTGGGACCGGGGCCAACACTGGCAGCTGGACATGGCGGACACGAGATCCGTGCACGAGACTAGGTT
TGAGGCGGCCGTGAAGGTGATCCAGAGTTTGCCGAAGAATGGTTCATTCCAGCCAACAAATGAAATGATGCTTAAATTTT
ATAGCTTCTATAAGCAGGCAACTGAAGGACCCTGTAAACTTTCAAGGCCTGCATTTTGGGATCCTATTGCAAGATATAAA
TGGGATGCTTGGAGTTCACTGGGTGATATGACCAAAGAGGAAGCCATGATTGCATATGTTGAAGAAATGAAAAAGATTAT
TGAAACTATGCCAATGACTGAGAAAGTTGAAGAATTGCTGCGTGTCATAGGTCCATTTTATGAAATTGTCGAGGACAAAA
AGAGTGGCAGGAGTTCTGATATAACCTCAGATCTTGGTAATGTTCTCACTTCTACTCCGAACGCCAAAACCGTTAATGGT
AAAGCTGAAAGCAGTGACAGTGGAGCCGAGTCTGAGGAAGAAGAGGCCCAAGAAGAAGTGAAAGGAGCAGAACAAAGTGA
TAATGATAAGAAAATGATGAAGAAGTCAGCAGACCATAACAATTTGGAAGTCATTGTCACTAATGGCTATGATAAAGATG
GCTTTGTTCAGGATATACAGAATGACATTCATGCCAGTTCTTCCCTGAATGGCAGAAGCACTGAAGAAGTAAAGCCCATT
GATGAAAACTTGGGGCAAACTGGAAAATCTGCTGTTTGCATTCACCAAGATATAAATGATGATCAIGTTGAAGATGTTAC
AGGAATTCAGCATTTGACAAGCGATTCAGACAGTGAAGTTTACTGTGATTCTATGGAACAATTTGGACAAGAACAGTCTT
TAGACAGCTTTACGTCCAACAATGGACCATTTCAGTATTACTTGGGTGGTCATTCCAGTCAACCCATGGAAAATTCTGGA
TTTCGTGAAGATATTCAAGTACCTCCTGGAAATGGCAACATTGGGAATATGCAGGTGGTTGCAGTTGAAGGAAAAGGTGA
AGTCAAGCATGGAGGAGAAGATGGCGGGAATAACAGCGGAGCACCACACCGGGAGAAGCGAGGCGGAGAAACTGACGAAT
TCTCTAATGTTAGAAGAGGAAGAGGACATAGGATGCAACACTTGAGCGAAGGAACCAAGGGCCGGCAGGTGGGAAGTGGA
GGTGATGGGGAGCGCTGGGGCTCCGACAGAGGGTCCCGAGGCAGCCTCAACGAGCAGATCGCCCTCGTGCTGATGAGACT
GCAGGAGGACATGCAGAATGTCCTTCAGAGACTGCAGAAACTGGAAACGCTGACTGCTTTGCAGGCAAAATCATCAACAT
CAACATTGCAGACTGCTCCTCAGCCCACCTCACAGAGACCATCTTGGTGGCCCTTCGAGATGCCCTCTCGAG

TABLE 26G
Encoded NOV26b protein sequence.

(SEQ ID NO: 250)

KLDRPWDRGGHWQLEMANTRSVIETRFEAAVKVIQSLPKNGSFQPTNEMMLKFYSFYKQATEGPCKLSRPGFWDPIGPYKWD
AWSSLGDMTKEEANIAYVEEMKKTIETMPMTEKVEELLRVIGPFYETVEDKKSGRSSDTTSDLGNVLTSTPNAKTVNGKAES
SDSGAESEEEEAGEEVKGAEQSDNDKKMMKKSADHKNLEVIVTNGYDKDGFVQDIQNKIHASSSLNGRSTEEVKPIDENLGG
TGKSAVCIHQDINDDHVEDVTGIQHLTSDSDSEVYCDSMEQFGGEESLDSFTSNNGPFQYYLGGHSSQPMENSGFREDIQVP
PGNGNIGNMQVVAVEGKGEVKHGGEDGGNNSGAPHREKRCGETDEFSNVRRGRGHRMQHLSEGTKGRQVGSGGDGERWGSDR
GSPAAGSLNEQIALVLMRLQEDMQNVLQRLQKLETLTALQAKSSTSTLQTAPQPTSQRPSWWPFAAMPSR

NOV26c

In an alternative embodiment, a NOV26 variant is NOV26c of 1401 nucleotides (also referred to as CG51523-05_{—}164732479), shown in Table 26H. A NOV26c variant differs from NOV26a at positions 71, 170, 313, and 403, and by an insertion of 11 amino acids at positions 161-162.

TABLE 26H
NOV26c nucleotide sequence.

(SEQ ID NO: 251)

AAGCTTACTAGGTTTGAGGCGGCCGTGAAGGTGATCCAGAGTTTGCCGAAGAATGGTTCATTCCAGCCAACAAATGAAAT
GATGCTTAAATTTTATAGCTTCTATAAGCAGGCAACTGAAGGACCCTGTAAACTTTCAAGGCCTGGATTTTGGGATCCTA
TTGGAAGATATAAATGGGATGCTTGGAGTTCACTGGGTGATATGACCAAAGGGGAAGCCATGATTGCATATGTTGAAGAA
ATGAAAAAGATTATTCAAACTATGCCAATGACTGAGAAAGTTGAAGAATTGCTGCGTGTCATAGGTCCATTTTATGAAAT
TGTCGAGGACAAAAAGAGTGGCAGGAGTTCTGATATAACCTCAGTCCGACTGCAGAAAATCTCTAAATGTTTAGAAGATC
TTGGTAATGTTCTCACTTCTACTCCGAACGCCAAAACCGTTAATCGTAAAGCTGAAAGCAGTCACAGTGGAGCCGAGTCT
GAGGAAGAAGAGGCCCAAGAAGAAGTGAAAGGAGCAGAACAAAGTGATAATGATAAGAAAATGATGAAGAAGTCAGCAGA
CCATAAGAATTTGGAAGTCATTGTCACTAATGGCTATCATAAAGATGGCTTTGTTCAGGATATACAGAATGACATTCATG
CCAGTTCTTCCCTGAATGGCAGAAGCACTGAAGAAGTAAAGCCCATTCATGAAAACTTGGGGCAAACTGGAAAATCTGCT
GTTTGCATTCACCAAGATATAAATGATGATCATGTTGAAGATGTTACAGCAATTCAGCATTTGACAAGCGATTCAGACAG
TGAAGTTTACTGTGATTCTATGGAACAATTTGGACAAGAAGAGTCTTTAGACAGCTTTACGTCCAACAATGGACCATTTC
AGTATTACTTGGGTGGTCATTCCAGTCAACCCATGGAAAATTCTGGATTTCGTGAATATATTCAAGTACCTCCTGGAAAT
GGCAACATTGCGAATATGCAGGTGGTTGCAGTTGAAGGAAAAGGTGAAGTCAAGCATGGAGGAGAAGATGGCAGGAATAA
CAGCGGAGCACCACACCGGGAGAAGCGAGGCGGAGAAACTGACGAATTCTCTAATGTTAGAAGAGGAAGAGGACATAGGA
TGCAACACTTGAGCGAAGGAACCAAGGGCCGGCAGGTGGGAAGTACACGTGATGGGGAGCGCTGGGGCTCCGACAGAGGG
TCCCGAGGCAGCCTCAATGAGCAGATCGCCCTCGTGCTGATGAGACTGCAGGAGCACATGCAGAATGTCCTTCAGAGACT
GCAGAAACTGGAAACGCTGACTGCTTTGCAGGCAAAATCATCAACATCAACATTGCAGACTGCTCCTCAGCCCACCTCAC
ACAGACCATCTTGGTGGCCCTTCGAGATGTCTCCTCTCGAG

TABLE 26I
Encoded NOV26c protein sequence.

(SEQ ID NO: 252)

KLTRFEAAVKVIQSLPKNGSFQPTNEMMLKFYSFYKQATEGPCKLSRPGFWDPIGRYKWDAWSSLGDMTKGEAMIAYVEE
MKKIIETMPMTEKVEELLRVIGPFYEIVEDKKSGRSSDITSVRLEKISKCLEDLGNVLTSTPNAKTVNGKAESSDSGAES
EEEEAGEEVKGAEQSDNDKKMNKKSADHKNLEVIVTNGYDBDGFVQDIQNDIHASSSLNGRSTEEVKPIDENLGGTGKSA
VCIHQDINDDHVEDVTGIQHLTSDSDSEVYCDSMEQFCGEESLDSFTSNNGPFQYYLGGHSSQPMENSGFREYIQVPPCN
NIGNMQVVAVEGKGEVKHGGEDGRNNSCAPHREKRGGETDEFSNVRRGRGHRMQHLSEGTKGRQVGSCGDGERWGSDRGS
RCSLNEQIALVLMRLQEDMQNVLQRLQKLETLTALQAKSSTSTLQTAPQPTSQRPSWNPFEMSPLE

NOV26d

In an alternative embodiment, a NOV26 variant is NOV26d of 1401 nucleotides (also referred to as CG51523-05_{—}164732506), shown in Table 26J. A NOV26d variant differs from NOV26a at positions 170, 292, and 403, and by the insertion of 11 amino acids at position 161-162.

TABLE 26J
NOV26d nucleotide sequence.

(SEQ ID NO: 253)

AAGCTTACTAGGTTTGAGGCGGCCGTGAAGGTGATCCAGAGTTTGCCGAAGAATGGTTCATTCCAGCCAACAAATGAAAT
GATGCTTAAATTTTATAGCTTCTATAAGCAGGCAACTGAAGGACCCTGTAAACTTTCAAGGCCTGGATTTTGGGATCCTA
TTGGAAGATATAAATGGGATGCTTGGAGTTCACTGGGTGATATGACCAAAGAGGAAGCCATGATTGCATATGTTGAAGAA
ATGAAAAAGATTATTGAAACTATGCCAATGACTGAGAAAGTTGAAGAATTGCTGCGTGTCATAGGTCCATTTTATGAAAT
TGTCGAGGACAAAAAGAGTGGCAGGAGTTCTGATATAACCTCAGTCCGACTGGAGAAAATCTCTAAATGTTTAGAAGATC
TTGGTAATGTTCTCACTTCTACTCCAAACGCCAAAACCGTTAATGGTAAAGCTGAAAGCAGTGACAGTGGAGCCGAGTCT
GAGGAAGAAGAGGCCCCAAGAAGAAGTGAAAGGAGCAGAACAAAGTGATAATGATAAGAAATGATGAAGAAGTCAGCAGA
CCATAAGAATTTGGAAGTCATTGTCACTAATGGCTATGATAAAGATGGCTTTGTTCAGGATATACAGAATGACATTCATG
GTTTGCATTCACCAAGATATAAATGATGATCATGTTGAAGATGTTACAGGAATTCAGCATTTGACAAGCGATTCAGACAG
TGAAGTTTACTGTGATTCTATGGAACAATTTGGACAAGAAGAGTCTTTAGACAGCTTTACGTCCCAACAATGGACAATTC
AGTATTACTTGGGTGGTCATTCCAGTCAACCCATGGAAAATTCTGGATTTCGTGAAGATATTCAAGTACCTCCTGGAAAT
GGCAACATTGGGAATATGCAGGTGGTTGCAGTTGAAGGAAAAGGTGAAGTCAAGCATGGAGGAGAAGATGGCAGGAATAA
CAGCGGAGCGCCACACCGGGAGAAGCGAGGCGGAGAAACTGATGAATTCTCTAATGTTAGAAGAGGAAGAGGACATAGGA
TGCAACACTTGAGCGAAGGAACCAAGGGCCGGCAGGTGGGAAGTGGAGGTGATGGGGAGCGCTGGGGCTCCGACAGAGGG
TCCCGAGGCAGCCTCAATGAGCAGATCGCCCTCGTGCTGATGAGACTGCAGGAGGACATGCAGAATGTCCTTCAGAGACT
GCAGAAACTGGAAACGCTGACTGCTTTGCAGGCAAAATCATCAACATCAACATTGCAGACTGCTCCTCAGCCCACCTCAC
AGAGACCATCTTGGTGGCCCTTCGAGATGTCTCCTCTCGAG

TABLE 26K
Encoded NOV26d protein sequence.

(SEQ ID NO: 254)

KLTRFEAAVKVIQSLPKNGSFQPTNEMMLKFYSFYKQATEGPCKLSRPGFWDPIGRYKWDAWSSLGDMTKEEAMIAYVEEM
KKIIETMPMTEKVEELLRVIGPFYEIVEDKKSGRSSDITSVRLEKISKCLEDLGNVLTSTPNAKTVNGKAESSDSGAESEE
EAGEEVKGAEQSDNDKKMMKKSADHKNLEVIVTNGYDKDGFVQDIQNDIHASSSLNGRSTEEVKPIDENLGGTGKSAVCIH
QDINDDHVEDVTGIQHLTSDSDSEVYCDSMEQFGGEESLDSFTSNNGGFQYYLGGHSSQPMENSGFREDIQVPPGNNIGNM
QVVAVEGKGEVKHGGEDGRNNSGAPHREKRGGETDEFSNVRRGRdHRMQHLSEGTKGRQVGSGGDGERWGSDRGSRGSLNE
QIALVLMRLQEDMQNVLQRLQKLETLTALQAKSSTSTLQTAPQPTSQRPSAAPFEMSPLE

NOV26e

In an alternative embodiment, a NOV26 variant is NOV26e of 1401 nucleotides (also referred to as CG51523-05_{—}164732693), shown in Table 26L. A NOV26e variant differs from NOV26a at the protein level at positions 170 and 403, and by the insertion of 11 amino acids at position 161-162.

TABLE 26L
NOV26e nucleotide sequence.

(SEQ ID NO: 255)

AAGCTTACTAGGTTTGAGGCGGCCGTGAAGGTGATCCAGAGTTTGCCGAAGAATGGTTCATTCCAGCCAACAAATGAAAT
GATGCTTAAATTTTATAGCTTCTATAAGCAGGCAACTGAAGGACCCTGTAAACTTTCAAGGCCTGGATTTTGGGATCCTA
TTGGAAGATATAAATGGGATGCTTGGAGTTCACTGGGTGATATGACCAAAGAGGAAGCCATGATTGCATATGTTGAAGAA
ATGAAAAAGATTATTGAAACTATGCCAATGACTGAGAAAGTTGAAGAATTGCTGCGTGTCATAGGTCCATTTTATGAAAT
TGTCGAGGACAAAAAGAGTGGCAGGAGTTCTGATATAACCTCAGTCCGACTGGAGAAAATCTCTAAATGTTTAGAAGATC
TTGGTAATGTTCTCACTTCTACTCCAAACGCCAAAACCGTTAATGGTAAAGCTGAAAGCAGTGACAGTGGAGCCGAGTCT
GAGGAAGAAGAGGCCCAAGAAGAAGTGAAAGGAGCAGAACAAAGTGATAATGATAAGAAAATGATGAAGAAGTCACCAGA
CCATAAGAATTTGGAAGTCATTGTCACTAATGGCTATGATAAAGATGGCTTTGTTCAGGATATACAGAATGACATTCATG
CCAGTTCTTCCCTGAATGGCAGAAGCACTGAAGAAGTAAAGCCTATTGATGAAAACTTGGGGCAAACTGGAAAATCTGCT
GTTTGCATTCACCAAGATATAAATGATGATCATGTTGAAGATGTTACAGGAATTCAGCATTTGACAAGCGATTCAGACAG
TGAAGTTTACTGTGATTCTATGGAACAATTTGGACAAGAAGAGTCTTTAGACAGCTTTACGTCCAACAATGGACAATTTC
AGTATTACTTGGGTGGTCATTCCAGTCAACCCATGGAAAATTCTGGATTTCGTGAAGATATTCAAGTACCTCCTGGAAAT
GGCAACATTGGGAATATGCAGGTGGTTGCAGTTGAAGGAAAGGTGAAGTCAAGCATGGAGGAGAAGATGGCAGGGAATAA
CAGCGGAGCGCCACACCGGGAGAAGCCAGGCGGAGAAACTGATGAATTCTCTAATGTTAGAAGAGGAAGAGGACATAGGA
TGCAACACTTGAGCGAAGGAACCAAGGGCCGGCAGGTGGGAAGTGGAGGTGATGGGGAGCGCTGGGGCTCCGACAGAGGG
TCCCGAGGCAGCCTCAATGAGCAGATCGCCCTCGTGCTGATGAGACTGCAGGAGGACATGCAGAATGTCCTTCAGAGACT
GCAGAAACTGGAAACGCTGACTGCTTTGCAGGCAAAATCATCAACATCAACATTGCAGACTGCTCCTCAGCCCACCTCAC
AGAGACCATCTTGGTGGCCCTTCCGAGATGTCTCCTCTCGAG

TABLE 26M
NOV26e amino acid sequence.

(SEQ ID NO: 256)

KLTRFEAAVEQKVIQSLPKNGSFQPTNEMMLKFYKQATEGPCKLSRPGFWDPIGRYKWDAWSSLGDMTKEEGAMIAYVEEM
KKIIETMPMTEKVEELLRVIGPFYEIVEDKKSGRSSDITSVRLEKISKCLEDLEEEEAQEEVKGAEQSDNDKKMMKKSADH
KNLEVIVTNGYDKDGFVQDIQNDIHASSSLNGRSTEEVKPIDENLGQTGKSAVCIHQDINDDHVEDVTGIQHLTSDSDSEV
YCDSMEQFGQEESLDSTSNNGPFQYYLGGHSSQPMENSGFREDIQVPPGNNIGNMQVVAVEGKEGEVKHGGEDGRNNSGAP
HREKRGGETDEFSNVRRGRGHRMQHLSEGTKGRQVGSGGDGERWGSDRGSRGSLNEQIALVLMRLQEDMQNVLQRLQKLET
LTALQAKSSTSTLQTAPQPTSQRPSWWPFEMSPLE

NOV26f

In an alternative embodiment, a NOV26 variant is NOV26f of 1368 nucleotides (also referred to as CG51523-05_{—}164732709), shown in Table 26N. A NOV26f variant differs from NOV26a at the protein level at positions 170, 403, 449, and 485.

TABLE 26N
NOV26f nucleotide sequence.

(SEQ ID NO: 257)

AAGCTTACTAGGTTTGAGGCGGCCGTGAAGGTGATCCAGAGTTTGCCGAAGAATGGTTCATTCCAGCCAACAAATGAAAT
GATGCTTAAATTTTATAGCTTCTATAAGCAGGCAACTGAAGGACCCTGTAAACTTTCAAGGCCTGGATTTTGGGATCCTA
TTGGAAGATATAAATGGGATGCTTGGAGTTCACTGGGTGATATGACCAAAGAGGAAGCCATGATTGCATATGTTGAAGAA
ATGAAAAAGATTATTGAAACTATGCCAATGACTGAGAAAGTTGAAGAATTGCTGCGTGTCATAGGTCCATTTTATGAAAT
TGTCGAGGACAAAAAGAGTGGCAGGAGTTCTGATATAACCTCAGATCTTGGTAATGTTCTCACTTCTACTCCGAACGCCA
AAACCGTTAATGGTAAAGCTGAAAGCAGTGACAGTGGAGCCGAGTCTGAGGAAGAAGAGGCCCAAGAAGAAGTGAAAGGA
GCAGAACAAAGTGATAATGATAAGAAAATGATGAAGAAGTCAGCAGACCATAAGAATTTGGAAGTCATTGTCACTAATGG
CTATGATAAAGATGGCTTTGTTCAGGATATACAGAATGACATTCATGCCAGTTCTTCCCTGAATGGCAGAAGCACTGAAG
AAGTAAAGCCCATTGATGAAAACTTGGGGCAAACTGGAAAATCTGCTGTTTGCATTCACCAAGATATAAATGATGATCAT
GTTGAAGATGTTACAGGAATTCAGCATTTGACGAGCGATTCAGACAGTGAAGTTTACTGTGATTCTATGGAACAATTTGG
ACAAGAAGAGTCTTTAGACAGCTTTACGTCCAACAATGGACCATTTCAGTATTACTTGGGTGGTCATTCCAGTCAACCCA
TGGAAAATTCTGGATTTCGTGAAGATATTCAAGTACCTCCTGGAAATGGCAACATTGGGAATATGCAGGTGGTTGCAGTT
GAAGGAAAAGGCGAAGTCAAGCATGGAGGAGAAGATGGCAGGAATAACAGCGGAGCACCACACCGGGAGAAGCGAGGCGG
AGAAACTGACGAATTCTCTAATGTTAGAAGAGGAAGAGGACATAGGATGCAACACTTGAGCGAAGGAACCAAGGGCCGGC
AGGTGGGAAGTGGAGGTGATGGGGAGCGCTGGGGCTCCGACAGAGGGTCCCGAGGCAGCCTCAATGAGCAGATCGCCCTC
GTGCTGATGAGACTGCAGGAGGACATACAGAATGTCCTTCAGAGACTGCAGAAACTGGAAACGCTGACTGCTTTGCGGGC
AAAATCATCAACATCAACATTGCAGACTGCTCCTCAGCCCACCTCACAGAGATCATCTTGGTGGCCCTTCGAGATGTCTC
CTCTCGAG

TABLE 26O
Encoded NOV26f protein sequence.

(SEQ ID NO: 258)

KLTRFEAAVKVIQSLPKNGSFQPTNEMMLKFYSFYKQATEGPCKLSRPGFWDPIGRYKWDAWSSLGDMTKEEMIAYVEEM
KKIIETMPMTEKVEELLRVIGPFYEIVEDKKSGRSSDITSDLGNVLTSTPNAKTVNGKAESSDSGAESEEEAQEEVKGEQ
SDNDKKMMKKSADHKNLEVIVTNGYDKDGFVQDIQNDIHASSSLNGRSTEEVKPIDENLGQTGKSAVCIHQDINDDEDVT
GIQHLTSDSDSEVYCDSMEGFGQEESLDSFTSNNGPFQYYLGGHSSQPMENSGFREDIQVPPGNGNIGNMQVAVGKGEVK
HGGEDGRNNSGPHREKRGGETDEFSNVRRQRGHRMQHLSEGTKGRQVGSGGDGERWQSDRGSRGSLNEQIALVLMRLQED
IQNVLQRLQKLETLTALQAKSSTSTLQTAPQPTSQRSSWWPFEMSPLE

NOV26g

In an alternative embodiment, a NOV26 variant is NOV26g of 1586 nucleotides (also referred to as CG51523-05_{—}164718189), shown in Table 26P. A NOV26g variant differs from NOV26a by 2 amino acids at positions 170 and 403.

TABLE 26P
NOV26g nucleotide sequence.

(SEQ ID NO: 259)

AAGCTTCCACCATGTTCCAGTTTCATGCAGGCTCTTGGGAAAGCTGGTGCTGCTGCTGCCTGATTCCCGCCGACAGACCT
TGGGACCGGGGCCAACACTGGCAGCTGGAGATGGCGGACACGAGATCCGTGCACGAGACTAGGTTTGAGGCGGCCGTGAA
GGTGATCCAGAGTTTGCCGAAGAATGGTTCATTCCAGCCAACAAATGAAATGATGCTTAAATTTTATAGCTTCTATAAGC
AGGCAACTGAAGGACCCTGTAAACTTTCAAGGCCTGGATTTTGGGATCCTATTGGAAGATATAGATAAGATGCTTGGAGT
TCACTGGGTGATATGACCAAAGAGGAAGCCATGATTGCATATGTTCAAGAAATGAAAAAGATTATTGAAACTATGCGAAT
GACTGAGAAAGTTGAAGAATTGCTGCGTGTCATAGGTCCATTTTATGAAATTGTCGAGGACAAAAAGAGTGGAAGGAGTT
CTGATATAACCTCAGATCTTGGTAATGTTCTCACTTCTACTCCGAACGCCAAAACCGTTAATGGTAAAGCTGAAAGCAGT
GACAGTGGAGCCGAGTCTGAGGAAGAAGAGGCCCAAGAAGAAGTGAAAGGAGCAGAACAAAGTGATAATGATAAGAAAAT
GATGAAGAGTCAGCAGACCATAAGAATTTGGAAGTCATTGTCACTAATGGCTATGATAAAGATGGCTTTGTTCAGATTTA
TACAGAATGACATTCATGCCAGTTCTTCCCTGAATGGCAGAAGCACTGAAGAAGTAAAGCCCATTGATGAAAACTTGGGG
CAAACTGGAAAATCTGCTGTTTGCATTCACCAAGATATAAATGATGATCATGTTGAAGATGTTACAGGAATTCAGCATTT
GACAAGCGATTCAGACAGTGAAGTTTACTGTGATTCTATGGAACAATTTGQACAAGAAGAGTCTTTAGAGGGCTTTACGT
CCAACAATGGACCATTTCAGTATTACTTGGGTGGTCATTCCAGTCAACCCATGGAAAATTCTGGATTTCGTGAAGATATT
CAAGTACCTCCTGGAAATGGCAACATTGGGAATATGCAGGTGGTTGCAGTTGAAGGAAAAGGTGAAGTCAAGCATGGAGG
AGAAGATGGCAGGAATAACAGCGGAGCACCACACCGGGAGAAGCGAGGCGGAGAAACTGACGAATTCTCTAATGTTAGAA
GAGGAAGAGGACATAGGATGCAACACTTGAGCGAAGGAACCAAGGGCCGGCAGGTGGGAAGTGGAGGTGATGGGGAGCGC
TGQGGCTCCGACAGAGGTCCCGAGGCAQCCTcATCAGCAGATCGCCCTCGTGCTGATGAGACTGCTGCAGAGGACATGCA
GAATGTCCTTCAGAGACTGCAGAAACTGGAAACGCTGACTGCTTTGCAGGCAAAATCATCAACATCAACATTGCAGACTG
CTCCTCAGCCCACCTCACAGAGACCATCTTGGTGGCCCTTCGAGATGTCTCCTGGTGTGCTAACGTTTGCCATCATATGG
CCTTTTATTGCACAGTGGTTGGTGTATTTATACTATCAAAGAAGGAGAAGAAAACTGAACCTCGAG

TABLE 26Q
Encoded NOV26g protein sequence.

(SEQ ID NO: 260)

ASTMFQFHAGSWESWCCCCLIPADRPWDRGQHWQLEMADTRSVHETRFEAAVKVIQSLPKNGSFQPTNEMMLKFYSFYKQ
ATEGPCKLSRPGFWDPIGRYKWDAWSSLGDMTKEEAMIAYVEEMKKIIETMPMTEKVEELLRVIGPFYEIVEDKKSGRSS
DITSDLGNLTSTPNAKTVNGKAESSDSCAESEEEEAQEEVKQAEQSDNDKKMMKKSADHKNLEVIVTNGYDKGDGFVQDI
QNDIHASSSLNGRSTEEVKPIDENLGQTGKSAVCIHQDINDDHVEDVTGIQHLTSDSDSEVYCDSMEQFGQEESLDSFTS
NNGPFQYYLGGHSSQPMENSGFREDIQVPPGNGNIGNMQVVAVEGKGEVKHGGEDGRNNSGAPHREKRGGETDEFSNVRR
QRGHRMQHLSEGTKGRQVGSGGDGERWGSDRGSRGSLNEQIALVLMRLQEDMQNVLQRLQKLETLTALQAKSSTSTLQTA
PQPTSQRPSWWPFEMSPGVLTFAIIWPFIAQWLVYLYYQRRRRKLNLE

NOV26h

In an alternative embodiment, a NOV26 variant is NOV26h of 1618 nucleotides (also referred to as CG51523-05_{—}164718193), shown in Table 26R. A NOV26h variant differs from NOV26a by the first twenty amino acids, and the 3 amino acids at positions 170, 182 and 403. In addition, NOV26h differs from NOV26a by the insertion of eleven amino acids at position 161-162.

TABLE 26R
NOV26h nucleotide sequence.

(SEQ ID NO: 261)

AAGCTTCCACCATGTTCCAGTTTCATGCAGGCTCTTGGGAAGCTGGTGCTGCTGCTGCCTGATTCCGCCGACAGACCTTG
GACCGGGQCCAACACTGGCAGCTGGAGATGGCGGACACGAGATCCGTGCACGAGACTAGGTTTGAGGCGGCCGTGAAGGT
CATCCAGAGTTTGCCGAAGAATGGTTCATTCCAGCCAACAAATGAAATGATGCTTAAATTTTATAGCTTCTATGGCAGQC
AACTGAAGGACCCTGTAAACTTTCAAGGCCTGGATTTTGGGATCCTATTGGAAGATATAAATGGGATGCTTGAGTTCACT
GGGTGATATGACCAAAGAGGAAGCCATGATTGCATATGTTGAACAAATGAAGATTATTGAAACTATAGGCCAATGACTGA
GAAAGTTGAAGAATTGCTGCGTGTCATAGGTCCATTTTATGAATTGTCGAGGACAAATCGAGTGGAAGGAGTTCTGATAT
AACCTCAGTCCGACTGGAGAAAATCTCTAAATGTTTAGAAGATCTTGGTAATGTTCTGACTTCTACTCTTACGCCAAAAC
CGTTAATGGTAAAGCTGAAGGCAGTGACAGTGGAGCCGAGTCTGAGGAAGAAGAGGCCAAGAAGAAGTGAAACGAGCAGA
ACAAAGTGATAATGATAAGAAAATGATGAAGAAGTCAGCAGACCATAAGAATTTGGAAGTCATTGTCACTAATGGCTATG
ATAAAGATGGCTTTGTTCAGGATATACAGAATGACATTCATGCCAGTTCTTCCCTGAATGGCAGAAGCACTGAAGAAGTA
AAGCCCATTCATCAAAACTTGGGGCAAACTGGAAAATCTGCTGTTTGCATTCACCAAGATATAAATGATGATCATGTTGA
GATGTTACGAATTCAGCATTTGACAAGCGATTCAGACAGTGAAGTTTACTGTGATTCTATGGAACAATTTTTTGGACAAG
AAGAGTCTTTAGACAGCTTTACGTCCAACAATGGACCATTTCAGTATTACTTGGOTGGTCATTCCAGTCAACCCATGGAA
AATTCTGGATTTCGTGAAGATATTCAAGTACCTCCTGGAAATCGCAACATTGGGAATATGCAGGTGGTTGCAGTTCAAGG
AAAAGGTGAAGTCAAGCATGGACGAGAAQATGGCAGGAATAACACCGGAGCACCACACCAGGACAAGCCAGCCGGAGAAA
CTGACGAATTCTCTAATGTTAGAAGAGGAAGAGGACATACGATGCAACACTTGAGCGAAGGAACCAAGGGCCGGCAGGTG
GGAAGTGGAGGTGATCGCGAGCGCTGCGGCTCCGACAGAGGGTCCCGAGGCAGCCTCAATGAGCAGATCGCCCTCGTGCT
GATGACACTGCAGGAGGACATGCAGAATGTCCTTCAGAGACTGCAGAAACTGGAAACGCTGACTGCTTTCCAGGCAAAAT
CATCAACATCAACATTGCAGACTGCTCCTCAGCCCACCTCACAGAGACCATCTTGGTGGCCCTTCGAGATGTCTCCTGGT
GTGCTAACGTTTGCCATCATATCGCCTTTTATTGCACAGTGGTTGGTGTATTTATACTATCAAAGAAGGAGAAGAAAACT
GAACCTCGAG

TABLE 26S
Encoded NOV26h protein sequence.

(SEQ ID NO: 262)

SFHHVPVSCRLLGKLVLLLPDSADRPWDRCWQLEMADTRSVHETRFEAAVICVIQSLPKNGSFQPTNEMMLKFYSFYKQA
TEGPCKLSRPOFWDPIGRYKWDAWSSLGDMTKEEANIAYVEEMKKIITMPMTEKVEELLRVIGPFYEIVEDKKSGRSSDI
TSVRLEKISKCLEDLGNVLTSTPNAKTNGKAEGSDSGAESEEEEAQEEVKGAEQSDNDRKMMKKSADHRNLEVIVTNGYD
DGFVQDIQNDIHASSSLNCRSTEEVKPIDENLGQTGKSAVCIHQDINUDHVEDVTGIQHLTSDSDSEVYCDSMEQFCQEE
SLDSFTSNNIGPFQYYLGGHSSQPMENSGFREDIQVPPGNGNIGNMQVVAVEGKGEVKHGGEDGRNNSGAPHREKGETDE
FSNRRGRGHRMQNLSEGTKGRQVGSGGDGERWGSDRGSRGSLNEQTALVLMRLQEDMQNVLQRLQKLETLTALQAKSSTS
TLQTAPQPTSQRPSWWPFEMSPGVLTFAIIWPFIAQWLVYLYYQRRRRKLNLE

NOV26i

In an alternative embodiment, a NOV26 variant is NOV26i of 1586 nucleotides (also referred to as CG51523-05_{—}164718197), shown in Table 26T. A NOV26i variant differs from NOV26a by 4 amino acids at positions 170, 403, 422 and 466.

TABLE 26T
NOV26i nucleotide sequence.

(SEQ ID NO: 263)

AAGCTTCCACCATGTTCCAGTTTCATGCAGGCTCTTGGGAAAGCTGGTGCTGCTGCTGCCTGATTCCCGCCGACAGACCT
TGGGACCGGGGCCAACACTGGCAGCTGGAGATGGCGGACACGAGATCCGTGCACGAGACTAGGTTTGAGGCGGCCGTGAA
GGTGATCCAGAGTTTGCCGAAGAATGGTTCATTCCAGCCAACAAATGAAATGATGCTTAAATTTTATAGCTTCTATAAGC
AGGCAACTGAAGGACCCTGTAAACTTTCAAGGCCTGGATTTTGGGATCCTATTGGAAGATATAAATGGGATGCTTGGAGT
TCACTGGGTGATATGACCAAAGAGGAAGCCATGATTGCATATGTTGAAGAAATGAAAAAGATTATTGAAACTATGCCAAT
GACTGAGAAAGTTGAAGAATTGCTGCGTGTCATAGGTCCATTTTATGAAATTGTCGAGGACAAAAAGAGTGGCAGGAGTT
CTGATATAACCTCAGATCTTGGTAATGTTCTCACTTCTACTCCGAACGCCAAAACCGTTAATGGTAAAGCTGAAAGCAGT
GACAGTGGAGCCGAGTCTGAGGAAGAAGAGGCCCAAGAAGAAGTGAAAGGAGCAGAACAAAGTGATAATGATAAGAAAAT
GATGAAGAAGTCAGCAGACCATAAGAATTTGGAAGTCATTGTCACTAATCGCTATCATAAAGATAGCTTTGTTCAGGATA
TACAGAATGACATTCATGCCAGTTCTTCCCTGAATGGCAGAAGCACTGAAGAAGTAAAGCCCATTGATCAAAACTTGGGG
CAAACTGGAAAATCTGCTGTTTGCATTCACCAAGATATAAATGATGATCATGTTGAAGATGTTACAGGAATTCAGCATTT
GACAAGCGATTCAGACAGTGAAGTTTACTGTGATTCTATGGAACAATTTGGACAAGAAGAGTCTTTAGACAGCTTTACGT
CCAACAATGGACCATTTCAGTATTACTTGGGTGGTCATTCCAGTCAACCCATGGAAAATTCTGGATTTCGTGAAGATATT
CAAGTACCTCCTCGAAATGGCAACATTGGGAATATGCAGGTGGTTGCAGTTGAAGGAAAAGGTGAAGTCAAGCATGGAGC
AGAAGATGGCAGGAATAACAGCCGACCACCACACCGGGAGAAGCGAGGCGGAGAAACTGACGAATTCTCTAATGTTACAA
GAGGAACAGGACATAGGATGCAACACTTGAGCCAAGGAACCAAGGGCCGGCAGGTGGGAAGTGGAGGTGATGGGCGGCGC
TGGGGCTCCGACAGAGGGTCCCGAGGCAGCCTCAATGAGCAGATCGCCCTCGTGCTCATGAGACTGCAGGAGGACATGCA
GAATGTCCTTCAGAGACTGCAGAAACTGGAAACGCTGACTGCTTTGCGGGCAAAATCATCAACATCAACATTGCAGACTG
CTCCTCAGCCCACCTCACAGAGACCATCTTGGTGGCCCTTCGAGATGTCTCCTGGTGTGCTAACGTTTGCCATCATATGG
CCTTTTATTGCACAGTGGTTGGTGTATTTATACTATCAAAGAAGGAGAAGAAAACTGAACCTCGAG

TABLE 26U
Encoded NOV26i protein sequence.

(SEQ ID NO: 264)

ASTMFQFHAGSWESWCCCCLIPADRPWDRGQHWQLEMADTRSVHETRFEAAVKVIQSLPKNGSFQPTNEMMLKFYSFYKQA
TEGPCKLSRPGFWDPIGRYKWDAWSSLGDMTKEEAMIAYVEEMKKIIETMPMTEFVEELLRVIGPFYEIVEDKKSGRSSDI
TSDLGNVLTSTPNAKTVNGKAESSDSGAESEEEEAQEEVKGAEQSDNDKKMMKKSADHKNLEVIVTNGYDKDGFVQDIQND
IHASSSLNGRSTEEVKPIDENLGQTGKSAVCIHQDINDDHVEDVTGIQHLTSDSDSEVYCDSMEQFGQEESLDSFTSNNGP
FQYYLGGHSSQPMENSGFREDIQVPPGNGNIGNMQVVAVEGKGEVKHGGEDGRNNSGAPHREKRGGETDEFSNVRRGRGHR
MQHLSEGTKGRQVGSGGDGGRWGSDRGSRGSLNEQIALVLMRLQEDMQNVLQRLQKLETLTALRAKSSTSTLQTAPQPTSQ
RPSWWPFEMSPGVLTFAITWPFIAQWLVYLYYQRRRRKLNLE

NOV26j

In an alternative embodiment, a NOV26 variant is NOV26j of 1517 nucleotides (also referred to as CG51523-05_{—}164718205), shown in Table 26V. A NOV26j variant differs from NOV26a by 4 amino acids at positions 35, 121, 170 and 403, and by a deletion of twenty-three amino acids at position 350.

TABLE 26V
NOV26j nucleotide sequence.

(SEQ ID NO: 265)

AAGCTTCCACCATGTTCCAGTTTCATGCAGGCTCTTGGGAAAGCTGGTGCTGCTGCTGCCTGATTCCCGCCGACAGACCT
TGGGACCGGGGCCAACACTGGCAGCTGGAGATGGTGGACACGAGATCCGTGCACGAGACTAGGTTTGAGGCGGCCGTGAA
GGTGATCCAGAGTTTGCCGAAGAATGGTTCATTCCAGCCAACAAATGAAATGATGCTTAAATTTTATAGCTTCTATAAGC
AGGCAACTGAAGGACCCTGTAAACTTTCAAGGCCTGGATTTTGGGATCCTATTGGAAGATATAAATGGGATGCTTGGAGT
TCACTGGGTGATATGACCAAAGAGGAAGCCATGATTGCATATGTTGAAGAAGTGAAAAAGATTATTGAAACTATGCCAAT
GACTGAGAAAGTTGAAGAATTGCTGCGTGTCATAGGTCCATTTTATGAAATTGTCGAGGACAAAAAGAGTGGCAGGAGTT
CTGATATAACCTCAGATCTTGGTAATGTTCTCACTTCTACGCCAAACGCCAAAACCGTTAATGGTAAAGCTGAAAGCAGT
GACAGTGGAGCCGAGTCTGAGGAAGAAGAGGCCCAAGAAGAAGTGAAAGGAGCAGAACAAAGTGATAATGATAAGAAAAT
GATGAAGAAGTCAGCAGACCATAAGAATTTGGAAGTCATTGTCACTAATGGCTATGATAAAGATGGCTTTGTTCAGGATA
TACAGAATGACATTCATGCCAGTTCTTCCCTGAATGGCAGAAGCACTGAAGAAGTAAAGCCCATTGATGAAAACTTGGGG
CAAACTGGAAAATCTGCTGTTTGCATTCACCAAGATATAAATGATGATCATGTTGAAGATGTTACAGGAATTCAGCATTT
GACAAGCGATTCAGACAGTGAAGTTTACTGTGATTCTATGGAACAATTTGGACAAGAAGAGTCTTTAGACAGCTTTACGT
CCAACAATGGACCATTTCAGTATTACTTGGGTGGTCATTCCAGTCAACCCATGGAAAATTCTGGATTTCGTGAAGATATT
CAAGTACCTCCTGGAAATGGCAGGAATAACAGCGGAGCACCACACCGGGAGAAGCGAGGCGGAGAAACTGACGAATTCTC
TAATGTTAGAAGAGGAAGAGGACATAGGATGCAACACTTGAGCGAAGGAACCAAGGGCCGGCAGGTGGGAAGTGGAGGTG
ATGGGGAGCGCTGGGGCTCCGACAGAGGGTCCCGAGGCAGCCTCAATGAGCAGATCGCCCTCGTGCTGATGAGACTGCAG
GAGGACATGCAGAATGTCCTTCAGAGACTGCAGAAACTGGAAACGCTGACTGCTTTGCAGGCAAAATCATCAACATCAAC
ATTGCAGACTGCTCCTCAGCCCACCTCACAGAGACCATCTTGGTGGCCCTTCGAGATGTCTCCTGGTGTGCTAACGTTTG
CCATCATATGGCCTTTTATTGCACAGTGGTTGGTGTATTTATACTATCAAAGAAGGAGAAGAAAACTGAACCTCGAG

TABLE 26W
Encoded NOV26j protein sequence.

(SEQ ID NO: 266)

ASTMFQFHAGSWESWCCCCLIPADRPWDRGQHWQLEMVDTRSVHETRFEAAVKVIQSLPKNGSFQPTNEMMLKFYSFYKG
ATEGPCKLSRPGFWDPIGRYKWDAWSSLGDMTKEEAMIAYVEEVKKIIETMPMTEKVEELLRVIGPFYEIVEDKKSGRSS
DITSDLGNVLTSTPNAKTVNGKAESSDSGAESEEEEAQEEVKGAEQSDNDKKMMKKSADHKNLEVIVTNGYDKDGFVQDI
QNDIHASSSLNGRSTEEVKPIDENLGQTGKSAVCIHQDINDDHVEDVTGIQHLTSDSDSEVYCDSMEQFGQEESLDSFTS
NNGPFQYYLGGHSSQPMENSGFREDIQVPPGNGRNNSGAPHREKRGGETDEFSNVRRGRGHRMQHLSEGTKGRQVGSGGD
QERWGSDRGSRGSLNEQIALVLMRLQEDMQNVLQRLQKLETLTALQAKSSTSTLQTAPQPTSQRPSWWPFEMSPGVLTFA
IIWPFIAQWLVYLYYQRRRRKLNLE

NOV26k

In an alternative embodiment, a NOV26 variant is NOV26k of 1361 nucleotides (also referred to as CG51523-05_{—}164718209), shown in Table 26X. A NOV26k variant differs from NOV26a by 68 amino acid deletion at position 208 and 2 amino acid changes. In addition, at position 162, an 11 amino acid sequence replaces an 18 amino acid sequence.

TABLE 26X
NOV26k nucleotide sequence.

(SEQ ID NO: 267)

AAGCTTCCACCATGTTCCAGTTTCATGCAGGCTCTTGGGAAAGCTGGTGCTGCTGCTGCCTGATTCCCGCCGACAGACCT
TGGGACCGGGGCCAACACTGCCACCTGGAGATGGCGGACACGAGATCCGTCCACCAGACTACGTTTGAGGCGGCCGTGAA
GGTGATCCAGAGTTTGCCGAAGAATGGTTCATTCCAGCCAACAAATGAAATGATGCTTAAATTTTATAGCTTCTATAAGC
AQGCAACTGAAGGACCCTGTAAACTTTCAAGGCCTOGATTTTCGGATCCTATTGGAAGATATAAATGGGATGCTTGGAGT
TCACTGGGTGATATGACCAAAGAGGAAGCCATGATTGCATATGTTGAAGAAATGAAAAAGATTATTGAAACTATGCCAAT
GACTGAGAAAGTTGAAGAATTGCTGCGTGTCATAGGTCCATTTTATGAAATTGTCGAGGACAAAAAGAGTGGCACGAGTT
CTGATATAACCTCAGTCCGACTGGAGAAAATCTCTAAATGTTTAGAAGCTGAAAGCAGTGACAGTGGAGCCGAGTCTGAG
GAAGAAGAGGCCCAAGAAGAAGTGAAAGGAGCAGAACAAAGTGATAATGATATAAATGATGATCATGTTGAAGATGTTAC
AGGAATTCAGCATTTGACAAGCGATTCAGACAGTGAAGTTTACTGTGATTCTATGGAACAATTTGGACAAGAAGAGTCTT
TAGACAGCTTTACGTCCAACAATGGACCATTTCAGTATTACTTGCGTGGTCATTCCAGTCAACCCATGGAAAATTCTGGA
TTTCGTGAAGATATTCAAGTACCTCCTGGAAATGGCAACATTGGGAATATGCAGGTGGTTGCAGTTGAAGGAAAAGGTGA
AGTCAAGCATGGAGGAGAAGATGGCAGGAATAACACCCGAGCGCCACACCCGGACAAGCGAGGCGGAGAAACTGATGAAT
TCTCTAATGTTAGAAGAGGAAGACGACATAGGATGCAACACTTGAGCGAAGCAACCAAGGGCCGGCAGGTGGGAAGTGGA
CGTGATGGGGAGCGCTGGCGCTCCGACAGAGGGTCCCGAGGCAGCCTCAATGAGCAGATCGCCCTCGTGCTGATGAGACT
GCAGGAGGACATGCAGAATGTCCTTCAGAGACTGCAGAAACTCGAAACGCTGACTGCTTTGCAGCCAAAATCATCAACAT
CAACATTGCAGACTGCTCCTCAGCCCACCTCACAGAGACCATCTTGGTGGCCCTTCGAGATGTCTCCTGGTGTGCTAACG
TTTGCCATCATATGCCCTTTTATTGCACAGTGGTTGGCGTATTTATACTATCAAAGAAGGAGAAGAAAACTGAACCTCGA
G

TABLE 26Y
Encoded NOV26k protein sequence.

(SEQ ID NO: 268)

ASTMFQPHACSWESWCCCCLIPADRPWDRGQHWQLEMADTRSVHETRFEAAVKVIQSLPKNCSFQPTNEMMLKFYSFYKQ
ATEGPCKLSRPGFWDPIGRYKWDAWSSLGDMTKEEAMIAYVEEMKKIIETMPMTEKVEELLRVICPFYEIVEDKKSGRSS
DITSVRLEKISKCLEAESSDSGAESEEEEAQEEVKGAEQSDNDINDDHVEDVTGIQHLTSDSDSEVYCDSMEQFGQEESL
DSFTSNNGPFQYYLGGHSSQPMENSGFREDIQVPPGNGNIGNMQVVAVEGKCEVKHGGEDGRNNSGAPHREKRGGETDEF
SNVRRGRGHRMQHLSEGTKGRQVGSGGDGERWGSDRCSRGSLNEQIALVLMRLQEDMQNVLQRLQKLETLTALQAKSSTS
TLQTAPQPTSQRPSWWPFEMSPGVLTFAIIWPFIAQWLAYLYYQRRRRKLNLEG

NOV26l

In an alternative embodiment, a NOV26 variant is NOV26l of 1619 nucleotides (also referred to as CG51523-05_{—}164718213), shown in Table 26Z. A NOV26l variant differs from NOV26a by 5 amino acid changes, and an 11 amino acid insertion at position 161-162.

TABLE 26Z
N0V26l nucleotide sequence.

(SEQ ID NO: 269)

AAGCTTCCACCATGTTCCAGTTTCATGCAGGCTCTTGGOAAAQCTGGTCCTCCTGCTGCCTGATTCCCCCCGACAGGCCT
TGGGACCGGGGCCAACACTGGCAGCTGGAGATGGCGGACACGAGATCCGTGCACGAGACTAGGTTTGAGGCGGCCGTGAA
GGTGATCCAGAGTTTGCCGAAGAATGGTTCATTCCAGCCAACAAATGAAATGATGCTTAAATTTTATACCTTCTATAAGC
AGGCAACTGAAGGACCCTGTAAACTTTCAAGGCCTGGATTTTGGGATCCTATTGGAAGATATAAATGGGATGCTTGGAGT
TCACTGGGTGATATGACCAAACAGGAAGCCATAATTGCATATGTTGAAGAAATGAAAAAGATTATTGAAACTATGCCAAT
GACTQAGAAAGTTGAAGAATTGCTGCGTGTCATAGGTCCATTTTATGAAATTGTCGAGGACAAAAAGAGTGGCAGGAGTT
CTGATATAACCTCAGTCCCACTGGAGAAAATCTCTAAATGTTTACAAGATCTTCGTAATCTTCTCACTTCTACTCCGAAC
GCCAAAACCGTTAATGGTAAAGCTGAAAGCAGTGACAGTGGAGCCGAGTCTGAGGAAGAAGAGGCCCAAGAAGAAGTGAA
AGGAGCAGAACAAAGTGATAATGATAAGAAAATGATCAAGAAGTCAGCAGACCATAAGAATTTGGAAGTCATTCTCACTA
ATGGCTATGATAAAGATGGCTTTGTTCAGGATATACAGAATGACATTCATGCCAGTTCTTCCCTGAATGGCAGAAGCACT
GAACAAGTAAAGCCCATTGATGAAAACTTGaGGCAAACTGGAAAATCTGCTGTTTGCATTCACCAAGATATAAATCATGA
TCATCTTGAAGATGTTACAGGAATTCAOCATTTGACAAGCGATTCAGACAGTGAAGTTTACTGTGATTCTATGGAACAAT
TTGGACAAGAAGAGTCTTTAGACAGCTTTACGTCCAACAATGGACCATTTCAGTATTACTTGGGTGGTCATTCCAGTCAA
CCCATGGAAAATTCTGGATTTCGTGAAGATATTCAAGTACCTCCTGGAAATGGCAACATTGGGAATATGCAGGTTGTTGC
AGTTGAAGGAAAAGGCGAAGTCAAGCATGGAGGAGAAGATGGCACGAATAACAGCGGAGCACCACACCGGGAGGAGCGAG
GCGGAGAAACTGACGAATTCTCTAATGTTAGAAGAGGAAGAGGACATAGGATGCAACACTTGAGCGAAGGAACCAAGGGC
CGGCAGGTGGGAAGTGGAGGTGATGGGGAGCGCTGGGGCTCCGACAGAGGGTCCCGAGGCAGCCTCAATGAGCATATCGC
CCTCGTGCTGATGAGACTGCAGGAGGACATGCAGAATGTCCTTCAGAGACTGCAGAAACTGGAAACGCTGACTGCTTTGC
AGGCAAAATCATCAACATCAACATTGCAGACTGCTCCTCAGCCCACCTCACAGAGACCATCTTGGTGGCCCTTCGAGATG
TCTCCTGGTGTGCTAACGTTTGCCATCATATGGCCTTTTATTGCACAGTGGTTGGTGTATTTATACTATCAAAGAAGGAG
AAGAAAACTGAACCTCGAG

TABLE 26AA
Encoded NOV26l protein sequence.

(SEQ ID NO: 270)

ASTMFQFHAGSWESWCCCCLIPADRPWDRGQHWQLEMADTRSVHETRFEAAVKVIQSLPKNGSFQPTNEMMLKFYSFYKQA
TEGPCKLSRPGFWDPIGRYKWDAWSSLGDMTKEEAIIAYVEEMKKIIETMPMTEKVEELLRVIGPFYEIVEDKKSGRSSD
ITSVRLEKISKCLEDLGNVLTSTPNKTVNGKAESSDSGAESEEEEAQEEVKGAEQSDNDKKMMKKSADHKNLEVIVTNGY
DKDGFVQDIQNKIHASSSLNGRSTEEVKPIDENLGQTGKSAVCIHQDINDDHVEDVTGIQHLTSDSDSEVYCDSMEQFGQ
EESLDSFTSNNGPFQYYLGGHSSQMENSGFREDIQVPPGNGNIGNMQVVAVEGKGEVKHGGEDGRNNSGAPHREERGGET
DEFSNVRRGRGHRMQHLSEGTKGRQVGSGGDGERWGSDRGSRGSLNEHIALVLMRLQEDMQNVLQRLQKLETLTALQAKS
STSTLQTAPQPTSQRPSWWPFEMSPGVLTFAIIWPFIAQWLVYLYYQRRRRKLNLE

NOV26m

In an alternative embodiment, a NOV26 variant is NOV26m of 1619 nucleotides (also referred to as CG51523-05_{—}166190452), shown in Table 26AB. A NOV26m variant differs from NOV26a by 4 amino acid changes, and an 11 amino acid insertion at position 161-162.

TABLE 26AB
NOV26m nucleotide sequence.

(SEQ ID NO: 271)

AAGCTTCCACCATGTTCCAGTTTCATGCAGGCTCTTGGGAAAGCTGGTGCTGCTGCTGCCTGATTCCCGCCGACAGACCT
TGGGACCGGGGCCAACACTGGCAGCTGGAGATGGCGGACACGAGATCCGTGCACGAGACTAGGTTTGAGGCGGCCGTGAA
GGTGATCCAGAGTTTGCCGAAGAATGGTTCATTCCAGCCAACAAATGAAGTGATGCTTAAATTTTATAGCTTCTATAAGC
AGGCAACTGAAGGACCCTGTAAACTTTCAAGGCCTGGATTTTGGGATCCTATTGGAAGATATAAATGGGATGCTTGGAGT
TCACTGGGTGATATGACCAAAGAGGAAGCCATGATTGCATATGTTGAAGAAATGAAAAAGATTATTGAAACTATGCCAAT
GACTGAGAAAGTTGAAGAATTGCTGCGTGTCATAGGTCCATTTTATGAAATTGTCGAGGACAAAAAGAGTGGCAGGAGTT
CTGATATAACCTCAGTCCGACTGGAGAAAATCTCTAAATGTTTAGAAGATCTTGGTAATGTTCTCACTTCTACTCCAAAC
GCCAAAACCGTTAATGGTAAAGCTGAAAGCAGTGACAGTGGAGCCGAGTCTGAGGAAGAAGAGGCCCAAGAAGAAGTGAA
AGGAGCAGAACAAAGTGATAATGATAAGAAAATGATGAAGAAGTCAGCAGACCATAAGAATTTGGAAGTCATTGTCACTA
ATGGCTATGATAAAGATGGCTTTGTTCAGGATATACAGAATGACATTCATGCCAGTTCTTCCCTGAATGGCAGAAGCACT
GAAGAAGTAAAGCCTATTGATGAAAACTTGGGGCAAACTGGAAAATCTGCTGTTTGCATTCACCAAGATATAAATGATGA
TCATGTTGAAGATGTTACAGGAATTCAGCATTTGACAAGCGATTCAGACAGTGAAGTTTACTGTGATTCTATGGAACAAT
TTGGACAAGAAGAGTCTTTAGACAGCTTTACGTCCAACAATGGACCATTTCAGTATTACTTGGGTGGTCATTCCAGTCAA
CCCATGGAAAATTCTGGATTTCGTGAAGATATTCAAGTACCTCCTGGAAATGGCAACATTGGGAATATGCAGGTGGTTGC
AGTTGAAGGAAAAGGTGAAGTCAAGCATGGAGGAGAAGATGGCAGGAATAACAGCGGAGCGCCACACCGGGAGAAGCGAG
GCGGAGAAACTGATGAATTCTCTAATGTTAGAAGAGGAAGAGGACATAGGATGCAACACTTGAGCGAAGGAACCAAGGGC
CGGCAGGTGGGAAGTGGAGATGATGGGGAGCGCTGGGGCTCCGACAGAGGGTCCCGAGGCAGCCTCAATGAGCAGATCGC
CCTCGTGCTCATGAGACTGCAGCAGGACATGCAGAATGTCCTTCAGAGACTGCAGAAACTGGAAACGCTGACTGCTTTGC
AGGCAAAATCATCAACATCAACATTGCAGACTGCTCCTCAGCCCACCTCACAGAGACCATCTTGGTGGCCCTTCGAGATG
TCTCCTGGTGTGCTAACGTTTGCCATCATATGGCCTTTTATTGCACAGTGGTTGGTGTATTTATACTATCAAAGAAGGAG
AAGAAAACTGAACCTCGAG

TABLE 26AC
hc,1 Encoded NOV26m protein sequence.

(SEQ ID NO: 272)

ASTMFQFHAGSWESWCCCCLIPADRPDRGQHWQLEMADTRSVHETRFEAAVKVIQSLPKNGSFQPTNEVMLKFYSFYKQA
TEGPCKLSRPGFWDPIGRYKWDAWSSLGDMTKEEAMIAYVEEMKKIIETMPMTEKVEELLRVIGPFYEIVEDKKSGRSSD
ITSVRLERISKCLEDLGNVLTSTPNAKTVNGKAESSDSGAESEEEEAQEEVKGAEQSDNDKKNMKKSADHKNLEVIVTNG
YDKDGFVQDIQNDIHASSSLNGRSTEEVKPIDENLGQTGKSAVCIHQDINDDHVEDVTGIQHLTSDSDSEVYCDSMEQFG
QEESLDSFTSNNGPFQYYLGGHSSQPMENSGFREDIQVPPGNGNIGNMQVVAVEGKGEVKHGGEDGRNNSGAPHREKRGG
ETDEFSNVRRGRGHRMQHLSEGTKGRQVGSGDDGERWGSDRGSRGSLNEQIALVLMRLQEDMQNVLQRLQKLETLTALQA
KSSTSTLQTAPQPTSQRPSWWPFEMSPGVLTFAIIWPEIAQWLVYLYYQRRRRKLNLE

NOV26n

In an alternative embodiment, a NOV26 variant is NOV26n of 1619 nucleotides (also referred to as CG51523-05_{—}166190467), shown in Table 26AD. Similarly to a NOV26n variant, a NOV26n variant differs from NOV26a by 4 amino acid changes, and an 11 amino acid insertion at position 161-162.

TABLE 26AD
NOV26n nucleotide sequence.

(SEQ ID NO: 273)

AAGCTTCCACCATGTTCCAGTTTCATGCAGGCTCTTGGGAAAGCTGGTGCTGCTGCTGCCTGATTCCCGCCGACAGACCT
TGGGACCGGGGCCCAAACTGGCAGCTGGAGATGGCGGACACGAGATCCGTGCACGAGACTAGGTTTGAGGCGGCCGTGAA
GGTGATCCAGAGTTTGCCGAAGAATGGTTCATTCCAGCCAACAAATGAAATGATGCTTAAATTTTATAGCTTCTATAAGC
AGGCAACTGAAGGACCCTGTAAACTTTCAAGGCCTGGATTTTGGGATCCTATTGGAAGATATAAATGGGATGCTTGGAGT
TCACTGGGTGATATGACCAAAGAGGAAGCCATGATTGCATATGTTGAAGAAATGAAAAAGATTATTGAAACTATGCCAAT
GACTGAGAAAGTTGAAGAATTGCTGCGTGTCATAGGTCCATTTTATGAAATTGTCGAGGACAAAAAGAGTGGCAGGAGTT
CTGATATAACCTCAGTCCGACTGGAGAAAATCTCTAAATGTTTAGAAGATCTTGGTAATGTTCTCACTTCTACTCCAAAC
GCCAAAACCGTTAATGGTAAAGCTGAAAGCAGTGACAGTGGAGCCGAGTCTGAGGAAGAAGAGGCCCAAGAAGAAGTGAA
AGGACCAGAACAAAGTGATAATGATAAGAAAATGATGAAGAAGTCAGCAGACCATAAGAATTTGGAAGTCATTGTCACTA
ATGGCTATGATAAGATGGCTTTGTTCAGGATATGCAGAATGACATTCATGCCAGTTCTTCCCTTGAATGGCAGAAGCACT
GAAGAAGTAAGGCCTATTGATGAAAACTTGGGGCAAACTGGAAAATCTGCTGTTTGCATTCACCAAGATATAAATGACGA
TCATGTTGAAGATGTTACAGGAATTCAGCATTTGACAAGCGATTCAGACAGTGAAGTTTACTGTGATTCTATGGAACAAT
TTGGACAAGAAGAGTCTTTAGACAGCTTTACGTCCAACAATGGACCATTTCAGTATTACTTGGGTGGTCATTCCAGTCAA
CCCATGGAAAATTCTGGATTTCGTGAAGATATTCAAGTACCTCCTGGAAATGGCAACATTGGGAATATGCAGGTGGTTGC
AGTTGAAGGAAAAGGTGAAGTCAAGCATGGAGGAGAAGATGGCAGGAATAACAGCGGAGCGCCACACCGGGAGAAGCGAG
GCGGAGAAACTGATGAATTCTCTAATGTTAGAAGAGGAAGAGGACATAGGATGCAACACTTGAGCGAAGGAACCAAGGGC
CGGCAGGTGGGAAGTGGAGGTGATGGGGAGCGCTGGGGCTCCGACAGAGGGTCCCGAGGCAGCCTCAATGAGCAGATCGC
CCTCGTGCTGATGAGACTGCAGGAGGACATCCAGAATGTCCTTCAGAGACTGCAGAAACTGGAAACGCTGACTGCTTTGC
AGGCAAAATCATCAACATCAACATTGCAGACTGCTCCTCAGCCCACCTCACAGAGACCATCTTGGTGGCCCTTCGAGATG
TCTCCTGGTGTGCTAACGTTTGCCATCATATGGCCTTTTATTGCACAGTGGTTGGTGTATTTATACTATCAAAGAAGGAG
AAGAAAACTGAACCTCGAG

TABLE 26AE
Encoded NOV26n protein sequence.

(SEQ ID NO: 274)

ASTMFQFHAGSWESWCCCCLIPADRPDRGQHWQLEMADTRSVHETRFEAAVKVIQSLPKNGSFQPTNEMMLKFYSFYKQA
TEGPCKLSRPGFWDPIGRYKWDAWSSLGDMTKEEANIAYVEEMKKIIETMPMTEKVEELLRVIGPFYEIVEDKKSGRSSD
ITSVRLEKISKCLEDLGNVLTSTPNAKTVNGKAESSDSGAESEEEEAQEEVKQAEQSDNDKKMMKKSADHKNLEVIVTNG
YDKDGFVQDMQNDIHASSSLNGRSTEEVRPIDENLGQTGKSAVCIHQDINDDHVEDVTGIQHLTSDSDSEVYCDSMEQPG
QEESLDSFTSNNGPFQYYLGGHSSQPMENSQFREDIQVPPGNGNIGNMQVVAVEGKGEVKHGGEDGRNNSGAPHREKRGG
ETDEFSNVRRGRGHRMQHLSEGTKGRQVGSGGDGERWGSDRGSRGSLNEQIALVLMRLQEDMQNVLQRLQKLETLTALQA
KSSTSTLQTAPQPTSQRPSWWPFEMSPGVLTFAIIWPFIAQWLVYLYYQRRRRKLNLE

NOV26o

In an alternative embodiment, a NOV26 variant is NOV26o of 1619 nucleotides (also referred to as CG51523-05_{—}166190475), shown in Table 26AF. A NOV26o variant differs from NOV26a by 3 amino acid changes at positions 170, 372 and 403, and an 11 amino acid insertion at position 161-162.

TABLE 26AF
NOV26o nucleotide sequence.

(SEQ ID NO: 275)

AAGCTTCCACCATGTTCCAGTTTCATGCAGGCTCTTGGGAAAGCTGGTGCTGCTGCTGCCTGATTCCCGCCGACAGACCT
TGGGACCGGGGCCAACACTGGCAGCTGGAGATGGCGGACACGAGATCCGTGCACGAGACTAGGTTTGAGGCGGCCGTGAA
GGTGATCCAGAGTTTGCCGAAGAATGGTTCATTCCAGCCAACAAATGAAATGATGCTTAAATTTTATAGCTTCTATAAGC
AGGCAACTGAAGGACCCTGTAAACTTTCAAGGCCTGGATTTTGGGATCCTATTGGAAGATATAAATGGGATGCTTGGAGT
TCACTGGGTGATATGACCAAAGAGGAAGCCATGATTGCATATGTTGAAGAAATGAAAAAGATTATTGAAACTATGCCAAT
GACTGAGAAAGTTGAAGAATTGCTGCGTGTCATAGGTCCATTTTATGAAATTGTCGAGGACAAAAAGAGTGGCAGGAGTT
CTGATATAACCTCAGTCCGACTGGAQAAAATCTCTAAATGTTTAGAAGATCTTGGTAATGTTCTCACTTCTACTCCGAAC
GCCAAAACCGTTAATGGTAAAGCTGAAAGCAGTGACAGTGGAGCCGAGTCTGAGGAAGAAGAGGCCCAAGAAGAAGTGAA
AGGAGCAGAACAAAGTGATAATGATAAGAAAATGATGAAGAAGTCAGCAGACCATAAGAATTTGGAAGTCATTGTCACTA
ATGGCTATGATAAAGATGGCTTTGTTCAGGATATACAGAATGACATTCATGCCAGTTCTTCCCTGAATGGCAGAAGCACT
GAAGAAGTAAAGCCCATTGATGAAAACTTGGGGCAAACTGGAAAATCTGCTGTTTGCATTCACCAAGATATAAATGATGA
TCATGTTGAAGATGTTACAGGAATTCAGCATTTGACAAGCGATTCAGACAGTGAAGTTTACTGTGATTCTATGGAACAAT
TTGGACAAGAAGAGTCTTTAGACAGCTTTACGTCCAACAATGGACCATTTCAGTATTACTTGGGTGGTCATTCCAGTCAA
CCCATGGAAAATTCTGGATTTCGTGAAGATATTCAAGTACCTCCTGGAAATGGCAACATTGGGAATATGCAGGTGGTTGC
AGTTGAAGGAAAAGGTGAAGTCAAGCATGGAGGAGAAGAGGGCAGGAATAACAGCGGAGCACCACACCGGGAGAAGCGAG
GCGGAGAAACTGACGAATTCTCTAATGTTAGAAGAGGAAGAGGACATAGGATGCAACACCTGAGCGAAGGAACCAAGGGC
CGGCAGGTGGGAAGTGGAGGTGATGGGGAGCGCTGGGGCTCCGACAGAGGGTCCCGAGGCAGCCTCAATGAGCAGATCGC
CCTCGTGCTGATGAGACTGCAGGAGGACATGCAGAATGTCCTTCAGAGACTGCAGAAACTGGAAACGCTGACTGCTTTGC
AGGCAAAATCATCAACATCAACATTGCAGACTGCTCCTCAGCCCACCTCACAGAGACCATCTTGGTGGCCCTTCGAGATG
TCTCCTGGTGTGCTAACGTTTGCCATCATATGGCCTTTTATTGCACAGTGGTTGGTGTATTTATACTATCAAAGAAGGAG
AAGAAAACTGAACCTCGAG

TABLE 26AG
Encoded NOV26o protein sequence.

(SEQ ID NO: 276)

ASTMFQFHAGSWESWCCCCLIPADRPWDRGQHWQLEMADTRSVHETRFEAAVKVIQSLPKNGSFQPTNEMMLKFYSFYKQ
ATEGPCKLSRPGFWDPIGRYKWDAWSSLGDMTKEEAMIAYVEEMKKIIETMPMTEKVEELLRVIGPFYEIVEDKKSGRSS
DITSVRLEKISKCLEDLGNVLTSTPNAKTVNGKAESSDSGAESEEEEAQEEVKGAEQSDNDKKMMKKSADHKNLEVIVTN
GYDKDGFVQDIQNDIHASSSLNGRSTEEVKPIDENLGQTGKSAVCIHQDINDDHVEDVTGIQHLTSDSDSEVYCDSMEQF
GQEESLDSFTSNNGPFQYYLGGHSSQPMENSGFREDIQVPPGNGNIGNMQVVAVEGKGEVKHGGEEGRNNSGAPHREKRG
GETDEFSNVRRGRGHRMQHLSEGTKGRQVGSGGDGERWGSDRGSRGSLNEQIALVLMRLQEDMQNVLQRLQKLETLTALQ
AKSSTSTLQTAPQPTSQRPSWWPFEMSPGVLTFAIIWPFIAQWLVYLYYQRRRRKLNLE

NOV26p

In an alternative embodiment, a NOV26 variant is NOV26p of 1619 nucleotides (also referred to as CG51523-05_{—}166190498), shown in Table 26AH. A NOV26p variant differs from NOV26a by 2 amino acid changes at positions 170 and 403, and an 11 amino acid insertion at position 161-162.

TABLE 26A11
NOV26p nucleotide sequence.

(SEQ ID NO: 277)

AAGCTTCCACCATGTTCCAGTTTCATGCAGGCTCTTGGGAAAGCTGGTGCTGCTGCTGCCTGATTCCCGCCGACAGACCT
TGGGACCGGGGCCAACACTGGCAGCTGGAGATGGCGGACACGAGATCCGTGCACGAGACTAGGTTTGAGGCGGCCGTGAA
GGTGATCCAGAGTTTGCCGAAGAATGGTTCATTCCAGCCAACAAATGAAATGATGCTTAAATTTTATAGCTTCTATAAGC
AGGCAACTGAAGGACCCTGTAAACTTTCAAGGCCTGGATTTTGGGATCCTATTGGAAGATATAAATGGGATGCTTGGAGT
TCACTGGGTGATATGACCAAAGAGGAAGCCATGATTGCATATGTTGAAGAAATGAAAAAGATTATTGAAACTATGCCAAT
GACTGAGAAAGTTGAAGAATTGCTGCGTGTCATAGGTCCATTTTATGAAATTGTCGAGGACAAAAAGAGTGGCAGGAGTT
CTGATATAACCTCAGTCCGACTGGAGAAAATCTCTAAATGTTTAGAAGATCTTGGTAATGTTCTCACTTCTACTCCGAAC
GCCAAAACCGTTAATGGTAAAGCTGAAAGCAGTGACAGGGAGCCGAGTCTGAGGAAGAAGAGGCCCAAGAAGAAGTGAAA
AGGAGCAGAACAAAGTGATAATGATAAGAAAATGATGAAGAAGTCAGCAGACCATAAGAATTTGGAAGTCATTGTCACTA
ATGGCTATGATAAAGATGGCTTTGTTCAGGATATACAATGACATTCATGCCAGTTCTTCCCTGAATGGCAGAAACGCACT
GAAGAAGTAAAGCCCATTGATGAAAACTTGGGGCAAACTGGAAAATCTGCTGTTTGCATTCACCAAGATATAAATGATGA
TCATGTTGAAGATGTTACAGGAATTCAGCATTTGACAAGCGATTCAGACAGTGAAGTTTACTGTGATTCTATGGAACAAT
TTGGACAAGAAGAGTCTTTAGACAGCTTTACGTCCAACAATGGACCATTTCAGTATTACTTGGGTGGTCATTCCAGTCAA
CCCATGGAAAATTCTGGATTTCGTGAAGATATTCAAGTACCTCCTGGAAATGGCAACATTGGGAATATGCAGGTGGTTGC
AGTTGAAGGAAAAGGTGAAGTCAAGCATGGAGGAGAAGATGGCAGGAATAACAGCGGAGCACCACACCGGGAGAAGCGAG
GCGGAGAAACTGACGAATTCTCTAATGTTAGAAGAGGAAGAGGACATAGGATGCAACACTTGAGCGAAGGAACCAAGGGC
CGGCAGGTGGGAAGTGGAGGTGATGGGGAGCGCTGGGGCTCCGACAGAGGGTCCCGAGGCAGCCTCAATGAGCAGATCGC
CCTCGTGCTGATGAGACTGCAGGAGGACATGCAGAATGTCCTTCAGAGACTGCAGAAACTGGAAACGCTGACTGCTTTGC
AGGCAAAATCATCAACATCAACATTGCAGACTGCTCCTCAGCCCACCTCACAGAGACCATCTTGGTGGCCCTTCGAGATG
TCTCCTGGTGTGCTAACGTTTGCCATCATATGGCCTTTTATTGCACAGTGGTTGGTGTATTTATACTATCAAAGAAGGAG
AAGAAAACTGAACCTCGAG

TABLE 26AI
Encoded NOV26p protein sequence.

(SEQ ID NO: 278)

ASTMFQFHAGSWESWCCCCLIPADRPWDRGQHWQLEMADTRSVHETRFEAAVKVIQSLPKNGSFQPTNEMMLKFYSFYKQ
ATEGPCKLSRPGFWDPIGRYKWDAWSSLGDMTKEEAMIAYVEEMKKIIETMPMTEKVEELLRVIGPFYEIVEDKKSGRSS
DITSVRLEKISKCLEDLGNSVLTSTPNAKTVNGKAESSDSGAESEEEEAQEEVKGAEQSDNKKMMKKSADHKNLEVIVTN
GYDKDGFVQDIQNDIHASSSLNGRSTEEVKPIDENLGQTGKSAVCIHQDINDDHVEDVTGIQHLTSDSDSEVYCDSMEQF
GQEESLDSFTSNNGPFQYYLGGHSSQPMENSGFREDIQVPPGNGNIGNMQVVAVEGKGEVKHGGEDGRNNSGAPHREKRG
GETDEFSNVRRGRGHRMQHLSEGTKGRQVGSGGDGERWGSDRGSRGSLNEQIALVLMRLQEDMQNVLQRLQKLETLTALQ
AKSSTSTLQTAPQPTSQRPSWWPFEMSPGVLTFAIIWPFIAQWLVYLYYQRRRRKLNLE

NOV26q

In an alternative embodiment, a NOV26 variant is NOV26q of 1586 nucleotides (also referred to as CG51523-05_{—}166190460), shown in Table 26AJ. A NOV26q variant differs from NOV26a by 3 amino acid changes at positions 170, 231 and 463.

TABLE 26AJ
NOV26q nucleotide sequence.

(SEQ ID NO: 279)

AAGCTTCCACCATGTTCCAGTTTCATGCAGGCTCTTGGGAAAGCTGGTGCTGCTGCTGCCTGATTCCCGCCGACAGACCT
TGGGACCGGGGCCAACACTGGCAGCTGGAGATGGCGGACACGAGATCCGTGCACGAGACTAGGTTTGAGGCGGCCGTGAA
GGTGATCCAGAGTTTGCCGAAGAATGGTTCATTCCAGCCAACAAATGAAATGATGCTTAAATTTTATAGCTTCTATAAGC
AGGCAACTGAAGGACCCTGTAAACTTTCAAGGCCTGGATTTTGGGATCCTATTGGAAGATATAAATGGGATGCTTGGAGT
TCACTGGGTGATATGACCAAAGAGGAAGCCATGATTGCATATGTTGAAGAAATGAAAAGATTATTGAAACTATGCCAATT
GACTGAGAAAGTTGAAGAATTGCTGCGTGTCATAGGTCCATTTTATGAAATTGTCGAGGACAAAAAGAGTGGCAGGAGTT
CTGATATAACCTCAGATCTTGGTAATGTTCTCACTTCTACTCCAAACGCCAAAACCGTTAATGGTAAAGCTGAAAGCAGT
GACAGTGGAGCCGAGTCTGAGGAAGAAGAGGCCCAAGAAGAAGTGAAAGGAGCAGAACAAAGTGATAATGATAAGAAATT
GATGAAGAAGTCAGCAGACCATAAGAATTTGGAAGTCATTGTCACTAATGGCTATGATAAAAATGGCTTTGTTCAGGATA
TACAGAATGACATTCATGCCAGTTCTTCCCTGAATGGCAGAAGCACTGAAGAAGTAAAGCCCATTGATGAAAACTTGGGG
CAAACTGGAAAATCTGCTGTTTGCATTCACCAAGATATAAATGATGATCATGTTGAAGATGTTACAGGAATTCAGCATTT
GACAAGCGATTCAGACAGTGAAGTTTACTGTGATTCTATGGAACAATTTGGACAAGAAGAGTCTTTAGACAGCTTTACGT
CCAACAATGGACCATTTCAGTATTACTTGGGTGGTCATTCCAGTCAACCCATGGAAAATTCTGGATTTCGTGAAGATATT
CAAGTACCTCCTGGAAATGGCAACATTGGGAATATGCAGGTGGTTGCAGTTGAAGGAAAAGGTGAAGTCAAGCATGGAGG
AGAAGATGGCAGGAATAACAGCGGAGCACCACACCGGGAGAAGCGAGGCGGAGAAACTGACGAATTCTCTAATGTTAGAA
GAGGAAGAGGACATAGGATGCAACACTTGAGCGAAGGAACCAAGGGCCGGCAGGTGGGAAGTGGAGGTGATGGGGAGCGC
TGGGGCTCCGACAGAGGGTCCCGAGGCAGCCTCAATGAGCAGATCGCCCTCGTGCTGATGAGACTGCAGGAGGACATGCA
GAATGTCCTTCAGAGACTGCAGAAACTGGAAACGCTGACTGCTTTGCAGGCAAATCATCAACATCAACATTGCAGACCTG
CTCCTCAGCCCACCTCACAQAGACCATCTTGGTGGCCCTTCGAGATGTCTCCTGGTGTGCTCGCGTTTGCCATCATATGG
CCTTTTATTGCACAGTGGTTGGTGTATTTATACTATCAAAGAAGGAGAAGAAAACTGAACCTCGAG

TABLE 26AK
Encoded NOV26q protein sequence.

(SEQ ID NO: 280)

ASTMFQFHAGSWESWCCCCLIPADRPWDRGQHWQLEMADTRSVHETRFEAAVKVIQSLPKNGSFQPTNEMMLKFYSFYKQA
TEGPCKLSRPGFWDPIGRYKWDAWSSLGDMTKEEAMIAYVEEMKKIIETMPMTEKVEELLRVIGPFYEIVEDKKSGRSSDI
TSDLGNVLTSTPNAKTVNGKAESSDSGAESEEEEAQEEVKGAEQSDNDKKMMKKSADHKNLEVIVTNGYDKNGFVQDIQND
IHASSSLNGRSTEEVKPIDENLGQTGKSAVCIHQDINDDHVEDVTGIQHLTSDSDSEVYCDSMEQFGQEESLDSFTSNNGP
FQYYLGGHSSQPMENSGFREDIQVPPGNGNIGNMQVVAVEGKGEVKHGGEDGRNNSGAPHREKRGGETDEFSNVRRGRGHR
MQHLSEGTKGRQVGSGGDGERWGSDRGSRGSLNEQIALVLMRLQEDMQNVLQRLQKLETLTALQAKSSTSTLQTAPQPTSQ
RPSWWPFEMSPGVLTFATIWPFIAQWLVYLYYQRRRRKLNLE

NOV26r

In an alternative embodiment, a NOV26 variant is NOV26r of 1586 nucleotides (also referred to as CG51523-05_{—}166190483), shown in Table 26AL. A NOV26r variant differs from NOV26a by 5 amino acid changes at positions 170, 342, 396, 403, and 452.

TABLE 26AL
NOV26r nucleotide sequence.

(SEQ ID NO: 281)

AAGCTTCCACCATGTTCCAGTTTCATGCAGGCTCTTCGGAAAGCTGGTGCTGCTGCTGCCTGATTCCCGCACGACGACCT
TGGGACCGGGGCCAACACTGGCAGCTGGAGATGGCGGACACGAGATCCGTGCACGAGACTAGGTTTGAGGCGGCCGTGAA
GGTGATCCAGAGTTTGCCGAAGAATGGTTCATTCCAGCCAACAAATGAAATGATGCTTAAATTTTATAGCTTCTATAAGC
AGGCAACTGAAGGACCCTGTAAACTTTCAAGGCCTGGATTTTGGGATCCTATTGGAAGATATAAATGGGATGCTTGGAGT
TCACTGGGTGATATGACCAAAGAGGAAGCCATGATTGCATATGTTGAAGAAATGAAAAAGATTATTGAAACTATGCCAAT
GACTGAGAAAGTTGAAGAATTGCTGCGTGTCATAGGTCCATTTTATGAAATTGTCGAAGACAAAAAGAGTGGCAGGAGTT
CTGATATAACCTCAGATCTTGGTAATGTTCTCACTTCTACTCCGAACGCCAAAACCGTTAATGGTAAAGCTGAAAGCAGT
GACAGTGGAGCCGAGTCTGAGGAAGAAGAGGCCCAAGAAGAAGTGAAAGGAGCAGAACAAAGTGATAATGATAAGAAAAT
GATGAAGAAGTCAGCAGACCATAAGAATTTGGAAGTCATTGTCACTAATGGCTATGATAAAGATGGCTTTGTTCAGGATA
TACAGAATGACATTCATGCCAGTTCTTCCCTGAATGGCAGAAGCACTGAAGAAGTAAAGCCCATTGATGAAAACTTGGGG
CAAACTGGAAAATCTGCTGTTTGCATTCACCAAGATATAAATGATGATCATGTTGAAGATGTTACAGGAATTCAGCATTT
GACAAGCGATTCAGACAGTGAAGTTACTGTGATTCTATGGAACAATTTGGACAAGAAGAGTCTTTAGACAGCTTTACCGT
CCAACAATGGACCATTTCAGTATTACTTGGGTGGTCATTCCAGTCAACCCATGGAAAATTCTGGATTTCGTGAATATATT
CAAGTACCTCCTGGAAATGGCAACATTGGGAATATGCAGGTGGTTGCAGTTGAAGGAAAAGGTGAAGTCAAGCATGGAGG
AGAAGATGGCAGGAATAACAGCGGAGCACCACACCGGAGAAGCGAGGCGGAGAAACTGACGAATTCTCTAATGTTTGGAA
GAGGAAGAGGACATAGGATGCAACACTTGAGCGAAGGAACCAAGGGCCGGCAGGTGGGAAGTGGAGGTGATGGGGAGCGC
TGGGGCTCCGACAGAGGGTCCCGAGGCAGCCTCAATGAGCAGATCGCCCTCGTGCTGATGAGACTGCAGGAGGACATGCA
GAATGTCCTTCAGAGACTGCAGAAACTGGAAACGCCGACTGCTTTGCAGGCAAAATCATCAACATCAACATTGCAGACTG
CTCCTCAGCCCACCTCACAGAGACCATCTTGGTGGCCCTTCGAGATGTCTCCTGGTGTGCTAACGTTTGCCATCATATGG
CCTTTTATTGCACAGTGGTTGGTGTATTTATACTATCAAAGAAGGAGAAGAAAACTGAACCTCGAG

TABLE 26AM
Encoded NOV26r protein sequence.

(SEQ ID NO: 282)

ASTMFQFHFAGSWESWCCCCLIPADRPWDRGQHWQLEMADTRSVHETRFEAAVKVIQSLPKNSFQPTNEMMLKFYSFYKQ
ATEGPCKLSRPGFWDPIGRYKWDAWSSLGDMTKEEAMIAYVEEMKKIIETMPMTEKVEELLRVIGPFYEIVEDKKSGRSS
DITSDLGNVLTSTPNAKTVNGKAESSDSGAESEEEEAQEEVKGAEQSDNDKKMMKKSADHKNLEVIVTNGYDKDGFVQDI
QNDIHASSSLNGRSTEEVKPIDENLGQTGKSAVCIHQDINDDHVEDVTGIQHLTSDSDSEVYCDSMEQFGQEESLDSFTS
NNGPFQYYLGGHSSQPMENSGFREYIQVPPGNGNIGNMQVVAVEGKGEVKHGGEDGRNNSGAPHREKRGGETDEFSNVGR
GRGHRMQHLSEGTKGRQVGSGGDGERWGSDRGSRGSLNEQIALVLMRLQEDMQNVLQRLQKLETPTALQAKSSTSTLQTA
QPTSQRPSWWPFEMSPGVLTFAIIWPFIAQWLVYLYYQRRRKLNLE

### Example B

### Sequencing Methodology and Identification of NOVX Clones

1. GeneCalling™ Technology:

This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology:

cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology:

The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof.

The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calf.) were then transferred from E. coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. No. 6,057,101 and 6,083,693).

4. RACE:

Techniques based on the polymerase chain reaction such as rapid amplification of cDNA ends (RACE), were used to isolate or complete the predicted sequence of the cDNA of the invention. Usually multiple clones were sequenced from one or ,more human samples to derive the sequences for fragments. Various human tissue samples from different donors were used for the RACE reaction. The sequences derived from these procedures were included in the SeqCalling Assembly process described in preceding paragraphs.

5. Exon Linking:

The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone:

Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISMS® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune/autoinflammatory diseases), Panel CNSD.01 (containing samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28 s: 18 s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-acfin and GAPDH). Normalized RNA (5 ul) was converted to cDNA and analyzed by RTQ-PCR using One Step RT-PCR Master Mix Reagents (Applied Biosystems; Catalog No.4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1× TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°−60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1× TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff pl effusion=pleural effusion,

glio=glioma,

astro=astrocytoma, and

neuro=neuroblastoma.

General_screening_panel_v1.4, v1.5 and v1.6

The plates for Panels 1.4, v1.5 and v1.6 include two control wells (genomic DNA control and chemistry control) and 94 wells containing cDNA from various samples. The samples in Panels 1.4, v1.5 and v1.6 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panels 1.4, v1.5 and v1.6 are widely available through the American Type Culture Collection (ATCC), a repository for cultured cell lines, and were cultured using the conditions recommended by the ATCC. The normal tissues found on Panels 1.4, v1.5 and v1.6 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D, 2.2, 2.3 and 2.4

The plates for Panels 2D, 2.2, 2.3 and 2.4 generally include two control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI) or from Ardais or Clinomics. The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI/CHTN/Ardais/Clinomics). Unmatched RNA samples from tissues without malignancy (normal tissues) were also obtained from Ardais or Clinomics. This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen. General oncology screening panel_v_{—}2.4 is an updated version of Panel 2D.

HASS Panel v 1.0

The HASS panel v 1.0 plates are comprised of 93 cDNA samples and two controls. Specifically, 81 of these samples are derived from cultured human cancer cell lines that had been subjected to serum starvation, acidosis and anoxia for different time periods as well as controls for these treatments, 3 samples of human primary cells, 9 samples of malignant brain cancer (4 medulloblastomas and 5 glioblastomas) and 2 controls. The human cancer cell lines are obtained from ATCC (American Type Culture Collection) and fall into the following tissue groups: breast cancer, prostate cancer, bladder carcinomas, pancreatic cancers and CNS cancer cell lines. These cancer cells are all cultured under standard recommended conditions. The treatments used (serum starvation, acidosis and anoxia) have been previously published in the scientific literature. The primary human cells were obtained from Clonetics (Walkersville, Md.) and were grown in the media and conditions recommended by Clonetics. The malignant brain cancer samples are obtained as part of a collaboration (Henry Ford Cancer Center) and are evaluated by a pathologist prior to CuraGen receiving the samples. RNA was prepared from these samples using the standard procedures. The genomic and chemistry control wells have been described previously.

ARDAIS Panel v 1.0

The plates for ARDAIS panel v 1.0 generally include 2 control wells and 22 test samples composed of RNA isolated from human tissue procured by surgeons working in close cooperation with Ardais Corporation. The tissues are derived from human lung malignancies (lung adenocarcinoma or lung squamous cell carcinoma) and in cases where indicated many malignant samples have “matched margins” obtained from noncancerous lung tissue just adjacent to the tumor. These matched margins are taken from the tissue surrounding (i.e. immediately proximal) to the zone of surgery (designated “NAT”, for normal adjacent tissue) in the results below. The tumor tissue and the “matched margins” are evaluated by independent pathologists (the surgical pathologists and again by a pathologist at Ardais). Unmatched malignant and non-malignant RNA samples from lungs were also obtained from Ardais. Additional information from Ardais provides a gross histopathological assessment of tumor differentiation grade and stage. Moreover, most samples include the original surgical pathology report that provides information regarding the clinical state of the patient.

Panels 3D, 3.1 and 3.2

The plates of Panel 3D, 3. 1, and 3.2 are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D, 3.1, 3.2, 1, 1.1, 1.2, 1.3D, 1.4, 1.5, and 1.6 are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells,, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1- 7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10−5 M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10−5 M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 μg/ml) were used to direct to Th 1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_Comprehensive Panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1 anti-trypsin deficiencies. Asthma patients ranged in age from 36-75, and excluded smokers to prevent those patients that could also have COPD. COPD patients ranged in age from 3 5-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22 /Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

AI.05 Chondrosarcoma

The AI.05 chondrosarcoma plates are comprised of SW1353 cells that had been subjected to serum starvation and treatment with cytokines that are known to induce MMP (1, 3 and 13) synthesis (eg. IL1beta). These treatments include: IL-1beta (10 ng/ml), IL-1beta+TNF-alpha (50 ng/ml), IL-1beta+Oncostatin (50 ng/ml) and PMA (100 ng/ml). The SW1353 cells were obtained from the ATCC (American Type Culture Collection) and were all cultured under standard recommended conditions. The SW1353 cells were plated at 3×10⁵ cells/ml (in DMEM medium—10% FBS) in 6-well plates. The treatment was done in triplicate, for 6 and 18 h. The supernatants were collected for analysis of MMP 1, 3 and 13 production and for RNA extraction. RNA was prepared from these samples using the standard procedures.

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

| | |
|---|---|
| Patient 2 | Diabetic Hispanic, overweight, not on insulin |
| Patient 7-9 | Nondiabetic Caucasian and obese (BMI > 30) |
| Patient 10 | Diabetic Hispanic, overweight, on insulin |
| Patient 11 | Nondiabetic African American and overweight |
| Patient 12 | Diabetic Hispanic on insulin |

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stern cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

A. CG103322-02: CD82 ANTIGEN.

Expression of gene CG103322-02 was assessed using the primer-probe set Ag6858, described in Table AA. Results of the RTQ-PCR runs are shown in Table AB. Please note that CG103322-02 represents a full-length physical clone.

TABLE AA
Probe Name Ag6858

| | | | Start | SEQ ID |
|---|---|---|---|---|
| Primers | Sequences | Length | Position | No |
| Forward | 5′-agaggacaacagcctttctgtg-3′ | 22 | 550 | 283 |
| Probe | TET-5′-caacaggacccagagtggcaaccac-3′-TAMRA | 25 | 598 | 284 |
| Reverse | 5′-ccaggagctcctggtacaca-3′ | 20 | 637 | 285 |

TABLE AB
General_screening_panel_v1.6

| | Rel. Exp. (%) |
|---|---|
| | Ag6858, Run |
| Tissue Name | 278387506 |
| Adipose | 1.0 |
| Melanoma* Hs688(A).T | 1.6 |
| Melanoma* Hs688(B).T | 0.6 |
| Melanoma* M14 | 7.0 |
| Melanoma* LOXIMVI | 27.5 |
| Melanoma* SK-MEL-5 | 2.5 |
| Squamous cell carcinoma SCC-4 | 60.3 |
| Testis Pool | 2.1 |
| Prostate ca.* (bone met) PC-3 | 1.7 |
| Prostate Pool | 3.6 |
| Placenta | 2.8 |
| Uterus Pool | 1.9 |
| Ovarian ca. OVCAR-3 | 27.0 |
| Ovarian ca. SK-OV-3 | 2.1 |
| Ovarian ca. OVCAR-4 | 1.7 |
| Ovarian ca. OVCAR-5 | 49.3 |
| Ovarian ca. IGROV-1 | 2.9 |
| Ovarian ca. OVCAR-8 | 5.6 |
| Ovary | 3.0 |
| Breast ca. MCF-7 | 0.0 |
| Breast ca. MDA-MB-231 | 44.8 |
| Breast ca. BT 549 | 7.4 |
| Breast ca. T47D | 48.0 |
| Breast ca. MDA-N | 0.3 |
| Breast Pool | 2.9 |
| Trachea | 9.5 |
| Lung | 1.7 |
| Fetal Lung | 5.9 |
| Lung ca. NCI-N417 | 0.1 |
| Lung ca. LX-1 | 11.0 |
| Lung ca. NCI-H146 | 6.9 |
| Lung ca. SHP-77 | 0.0 |
| Lung ca. A549 | 2.7 |
| Lung ca. NCI-H526 | 0.0 |
| Lung ca. NCI-H23 | 0.4 |
| Lung ca. NCI-H460 | 0.0 |
| Lung ca. HOP-62 | 3.4 |
| Lung ca. NCI-H522 | 0.2 |
| Liver | 0.0 |
| Fetal Liver | 9.6 |
| Liver ca. HepG2 | 0.0 |
| Kidney Pool | 1.5 |
| Fetal Kidney | 0.9 |
| Renal ca. 786-0 | 3.7 |
| Renal ca. A498 | 20.0 |
| Renal ca. ACHN | 0.8 |
| Renal ca. UO-31 | 100.0 |
| Renal ca. TK-10 | 1.5 |
| Bladder | 10.2 |
| Gastric ca. (liver met.) NCI-N87 | 44.8 |
| Gastric ca. KATO III | 20.3 |
| Colon ca. SW-948 | 9.0 |
| Colon ca. SW480 | 20.6 |
| Colon ca.* (SW480 met) SW620 | 8.2 |
| Colon ca. HT29 | 18.4 |
| Colon ca. HCT-116 | 13.4 |
| Colon ca. CaCo-2 | 4.9 |
| Colon cancer tissue | 23.0 |
| Colon ca. SW1116 | 4.0 |
| Colon ca. Colo-205 | 5.6 |
| Colon ca. SW-48 | 25.3 |
| Colon Pool | 1.6 |
| Small Intestine Pool | 3.7 |
| Stomach Pool | 1.4 |
| Bone Marrow Pool | 2.0 |
| Fetal Heart | 0.0 |
| Heart Pool | 1.1 |
| Lymph Node Pool | 0.0 |
| Fetal Skeletal Muscle | 2.1 |
| Skeletal Muscle Pool | 2.3 |
| Spleen Pool | 4.5 |
| Thymus Pool | 5.9 |
| CNS cancer (glio/astro) U87-MG | 55.1 |
| CNS cancer (glio/astro) U-118-MG | 23.5 |
| CNS cancer (neuro; met) SK-N-AS | 1.5 |
| CNS cancer (astro) SF-539 | 5.3 |
| CNS cancer (astro) SNB-75 | 11.9 |
| CNS cancer (glio) SNB-19 | 3.3 |
| CNS cancer (glio) SF-295 | 21.3 |
| Brain (Amygdala) Pool | 6.6 |
| Brain (cerebellum) | 5.1 |
| Brain (fetal) | 3.6 |
| Brain (Hippocampus) Pool | 5.8 |
| Cerebral Cortex Pool | 6.4 |
| Brain (Substantia nigra) Pool | 5.6 |
| Brain (Thalamus) Pool | 7.4 |
| Brain (whole) | 2.5 |
| Spinal Cord Pool | 8.4 |
| Adrenal Gland | 2.5 |
| Pituitary gland Pool | 1.4 |
| Salivary Gland | 6.0 |
| Thyroid (female) | 3.0 |
| Pancreatic ca. CAPAN2 | 0.9 |
| Pancreas Pool | 7.3 |

General_screening_panel_v1.6 Summary: Ag6858

The gene is expressed at low levels in most of the cancer cell lines on this panel with the highest expression in a renal cancer cell line UO-31 (CT=30.03). It may be used as a marker for expression.

CG103322-02 is a deletion splice variant of CD82/KAI1, a gene which was first described in the literature as a metastasis suppressor for prostate cancer (Dong, J.-T.; Lamb, P. W.; Rinker-Schaeffer, C. W.; Vukanovic, J.; Ichikawa, T.; Isaacs, J. T.; Barrett, J. C. KAI1, a metastasis suppressor gene for prostate cancer on human chromosome 11p 11.2. Science 268: 884-886, 1995.)

B. CG151575-02: Novel Multi-Pass Membrane Protein.

Expression of gene CGI51575-02 was assessed using the primer-probe set Ag7621, described in Table BA. Results of the RTQ-PCR runs are shown in Table BB.

TABLE BA
Probe Name Ag7621

| | | | Start | SEQ ID |
|---|---|---|---|---|
| Primers | Sequences | Length | Position | No |
| Forward | 5′-cccagagtatctcaagggactt-3′ | 22 | 219 | 286 |
| Probe | TET-5′-aagctgtctctgctgatagactccttcc-3′-TAMRA | 28 | 257 | 287 |
| Reverse | 5′-gtgagatcctgctgtgttgg-3′ | 20 | 304 | 288 |

TABLE BB
Panel 4.1D

| | Rel. Exp. (%) |
|---|---|
| | Ag7621, Run |
| Tissue Name | 311288444 |
| Secondary Th1 act | 5.9 |
| Secondary Th2 act | 33.7 |
| Secondary Tr1 act | 9.5 |
| Secondary Th1 rest | 0.0 |
| Secondary Th2 rest | 0.0 |
| Secondary Tr1 rest | 9.3 |
| Primary Th1 act | 0.0 |
| Primary Th2 act | 4.5 |
| Primary Tr1 act | 0.0 |
| Primary Th1 rest | 4.9 |
| Primary Th2 rest | 0.0 |
| Primary Tr1 rest | 0.0 |
| CD45RA CD4 lymphocyte act | 31.0 |
| CD45RO CD4 lymphocyte act | 12.3 |
| CD8 lymphocyte act | 0.0 |
| Secondary CD8 lymphocyte rest | 5.5 |
| Secondary CD8 lymphocyte act | 7.1 |
| CD4 lymphocyte none | 0.0 |
| 2ry Th1/Th2/Tr1_anti-CD95 CH11 | 0.0 |
| LAK cells rest | 4.5 |
| LAK cells IL-2 | 14.4 |
| LAK cells IL-2 + IL-12 | 0.0 |
| LAK cells IL-2 + IFN gamma | 0.0 |
| LAK cells IL-2 + IL-18 | 8.1 |
| LAK cells PMA/ionomycin | 6.7 |
| NK Cells IL-2 rest | 18.7 |
| Two Way MLR 3 day | 12.5 |
| Two Way MLR 5 day | 0.0 |
| Two Way MLR 7 day | 0.0 |
| PBMC rest | 4.2 |
| PBMC PWM | 0.0 |
| PBMC PHA-L | 0.0 |
| Ramos (B cell) none | 4.4 |
| Ramos (B cell) ionomycin | 7.3 |
| B lymphocytes PWM | 4.3 |
| B lymphocytes CD40L and IL-4 | 15.2 |
| EOL-1 dbcAMP | 0.0 |
| EOL-1 dbcAMP PMA/ionomycin | 0.0 |
| Dendritic cells none | 22.5 |
| Dendritic cells LPS | 2.9 |
| Dendritic cells anti-CD40 | 0.0 |
| Monocytes rest | 24.0 |
| Monocytes LPS | 41.2 |
| Macrophages rest | 15.2 |
| Macrophages LPS | 14.7 |
| HUVEC none | 5.6 |
| HUVEC starved | 12.4 |
| HUVEC IL-1beta | 4.1 |
| HUVEC IFN gamma | 4.9 |
| HUVEC TNF alpha + IFN gamma | 3.4 |
| HUVEC TNF alpha + IL4 | 0.0 |
| HUVEC IL-11 | 13.3 |
| Lung Microvascular EC none | 27.7 |
| Lung Microvascular EC TNFalpha + IL-1beta | 8.5 |
| Microvascular Dermal EC none | 25.2 |
| Microsvasular Dermal EC TNFalpha + IL-1beta | 0.0 |
| Bronchial epithelium TNFalpha + IL1beta | 43.5 |
| Small airway epithelium none | 23.3 |
| Small airway epithelium TNFalpha + IL-1beta | 71.7 |
| Coronery artery SMC rest | 0.0 |
| Coronery artery SMC TNFalpha + IL-1beta | 0.0 |
| Astrocytes rest | 14.8 |
| Astrocytes TNFalpha + IL-1beta | 19.5 |
| KU-812 (Basophil) rest | 18.3 |
| KU-812 (Basophil) PMA/ionomycin | 8.8 |
| CCD1106 (Keratinocytes) none | 56.6 |
| CCD1106 (Keratinocytes) TNFalpha + IL-1beta | 15.2 |
| Liver cirrhosis | 3.5 |
| NCI-H292 none | 15.5 |
| NCI-H292 IL-4 | 7.0 |
| NCI-H292 IL-9 | 31.4 |
| NCI-H292 IL-13 | 7.5 |
| NCI-H292 IFN gamma | 100.0 |
| HPAEC none | 5.7 |
| HPAEC TNF alpha + IL-1 beta | 17.8 |
| Lung fibroblast none | 16.7 |
| Lung fibroblast TNF alpha + IL-1 beta | 15.6 |
| Lung fibroblast IL-4 | 5.9 |
| Lung fibroblast IL-9 | 42.9 |
| Lung fibroblast IL-13 | 0.0 |
| Lung fibroblast IFN gamma | 17.0 |
| Dermal fibroblast CCD1070 rest | 24.8 |
| Dermal fibroblast CCD1070 TNF alpha | 58.2 |
| Dermal fibroblast CCD1070 IL-1 beta | 11.3 |
| Dermal fibroblast IFN gamma | 10.0 |
| Dermal fibroblast IL-4 | 61.1 |
| Dermal Fibroblasts rest | 21.5 |
| Neutrophils TNFa + LPS | 0.0 |
| Neutrophils rest | 0.0 |
| Colon | 0.0 |
| Lung | 17.1 |
| Thymus | 10.7 |
| Kidney | 23.8 |

CNS_neurodegeneration_v1.0 Summary: Ag7621 Expression of this gene is low/undetectable (CTs>35) across all of the samples on this panel.

Panel 4.1D Summary:

Ag7621 Low expression of this gene is detected mainly in IFN gamma treated NCI-H292 (CT=34.6). NCI-H292 cell line is a human airway epithelial cell line that produces mucins. Expression of this gene is higher in IFN gamma stimulated NCI-H292 compared to resting cells. Thus, this gene may be important in the proliferation or activation of airway epithelium. Mucus overproduction is an important feature of bronchial asthma and chronic obstructive pulmonary disease samples. Therefore, therapeutics designed with the protein encoded by the gene may reduce or eliminate symptoms caused by inflammation in lung epithelia in chronic obstructive pulmonary disease, asthma, allergy, and emphysema.

C. CG153011-01: Sushi Domain-Containing Membrane Protein.

Expression of gene CG153011-01 was assessed using the primer-probe set Ag6966, described in Table CA. Results of the RTQ-PCR runs are shown in Table CB. Please note that CG153011-01 represents a full-length physical clone.

TABLE CA

Probe Name Ag6966

| | | | | |
|---|---|---|---|---|
| | | | Start | SEQ ID |
| Primers | Sequences | Length | Position | No |

| | | | | |
|---|---|---|---|---|
| Forward | 5′-cagcgcagagaaatctcac-3′ | 19 | 170 | 289 |
| Probe | TET-5′-tcccaatcccgaggaaaaccagagaagtagct-3′-TAMRA | 32 | 213 | 290 |
| Reverse | 5′-agagtaatgtggcaccgtctc-3′ | 21 | 249 | 291 |

TABLE CB
General_screening_panel_v1.6

| | Rel. Exp. (%) |
|---|---|
| | Ag6966, Run |
| Tissue Name | 278388950 |
| Adipose | 0.7 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.2 |
| Squamous cell carcinoma SCC-4 | 0.0 |
| Testis Pool | 8.4 |
| Prostate ca.* (bone met) PC-3 | 0.0 |
| Prostate Pool | 9.1 |
| Placenta | 0.0 |
| Uterus Pool | 2.5 |
| Ovarian ca. OVCAR-3 | 52.9 |
| Ovarian ca. SK-OV-3 | 33.2 |
| Ovarian ca. OVCAR-4 | 9.9 |
| Ovarian ca. OVCAR-5 | 2.8 |
| Ovarian ca. IGROV-1 | 2.6 |
| Ovarian ca. OVCAR-8 | 12.1 |
| Ovary | 18.7 |
| Breast ca. MCF-7 | 47.0 |
| Breast ca. MDA-MB-231 | 0.0 |
| Breast ca. BT 549 | 17.9 |
| Breast ca. T47D | 2.1 |
| Breast ca. MDA-N | 0.0 |
| Breast Pool | 0.6 |
| Trachea | 13.9 |
| Lung | 7.1 |
| Fetal Lung | 23.8 |
| Lung ca. NCI-N417 | 24.1 |
| Lung ca. LX-1 | 0.0 |
| Lung ca. NCI-H146 | 0.0 |
| Lung ca. SHP-77 | 8.8 |
| Lung ca. A549 | 2.2 |
| Lung ca. NCI-H526 | 4.1 |
| Lung ca. NCI-H23 | 0.6 |
| Lung ca. NCI-H460 | 0.0 |
| Lung ca. HOP-62 | 0.0 |
| Lung ca. NCI-H522 | 1.7 |
| Liver | 0.0 |
| Fetal Liver | 0.4 |
| Liver ca. HepG2 | 0.0 |
| Kidney Pool | 2.0 |
| Fetal Kidney | 9.3 |
| Renal ca. 786-0 | 100.0 |
| Renal ca. A498 | 3.6 |
| Renal ca. ACHN | 4.3 |
| Renal ca. UO-31 | 0.0 |
| Renal ca. TK-10 | 17.4 |
| Bladder | 25.5 |
| Gastric ca. (liver met.) NCI-N87 | 68.3 |
| Gastric ca. KATO III | 0.0 |
| Colon ca. SW-948 | 0.0 |
| Colon ca. SW480 | 24.5 |
| Colon ca.* (SW480 met) SW620 | 0.0 |
| Colon ca. HT29 | 0.0 |
| Colon ca. HCT-116 | 1.3 |
| Colon ca. CaCo-2 | 62.0 |
| Colon cancer tissue | 0.8 |
| Colon ca. SW1116 | 0.0 |
| Colon ca. Colo-205 | 0.0 |
| Colon ca. SW-48 | 0.0 |
| Colon Pool | 0.9 |
| Small Intestine Pool | 1.2 |
| Stomach Pool | 2.9 |
| Bone Marrow Pool | 3.8 |
| Fetal Heart | 10.2 |
| Heart Pool | 1.3 |
| Lymph Node Pool | 0.7 |
| Fetal Skeletal Muscle | 1.3 |
| Skeletal Muscle Pool | 0.0 |
| Spleen Pool | 0.0 |
| Thymus Pool | 4.2 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 1.1 |
| CNS cancer (astro) SNB-75 | 17.6 |
| CNS cancer (glio) SNB-19 | 2.5 |
| CNS cancer (glio) SF-295 | 17.8 |
| Brain (Amygdala) Pool | 7.3 |
| Brain (cerebellum) | 35.6 |
| Brain (fetal) | 8.8 |
| Brain (Hippocampus) Pool | 14.5 |
| Cerebral Cortex Pool | 18.2 |
| Brain (Substantia nigra) Pool | 15.0 |
| Brain (Thalamus) Pool | 16.7 |
| Brain (whole) | 8.6 |
| Spinal Cord Pool | 9.1 |
| Adrenal Gland | 2.3 |
| Pituitary gland Pool | 4.3 |
| Salivary Gland | 9.5 |
| Thyroid (female) | 1.4 |
| Pancreatic ca. CAPAN2 | 0.0 |
| Pancreas Pool | 1.7 |

General_screening_panel_v1.6 Summary:

Ag6966 Highest expression of this gene is detected in a renal cancer 786-0 cell line (CT=30.8). Moderate levels of expression of this gene is also seen in cluster of cancer cell lines derived from gastric, colon, lung, renal, breast, ovarian, and brain cancers. Thus, expression of this gene could be used as a marker to detect the presence of these cancers. Furthermore, therapeutic modulation of the expression or function of this gene may be effective in the treatment of gastric, colon, lung, renal, breast, ovarian, and brain cancers.

In addition, this gene is expressed at moderate to low levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

D. CG153179-01: Membrane Protein.

Expression of gene CG153179-01 was assessed using the primer-probe set Ag6863, described in Table DA. Results of the RTQ-PCR runs are shown in Table DB. Please note that CG153179-01 represents a full-length physical clone.

TABLE DA

Probe Name Ag6863

| | | | Start | SEQ ID |
|---|---|---|---|---|
| Primers | Sequences | Length | Position | No |
| Forward | 5′-acgtgcaggtttgttacata-3′ | 20 | 609 | 292 |
| Probe | TET-5′-tgtgctacacccattaactcgtcatttaac-3′-TAMRA | 30 | 650 | 293 |
| Reverse | 5′-accttggcagggctaat-3′ | 17 | 680 | 294 |

TABLE DB
General_screening_panel_v1.6

| | Rel. Exp. (%) |
|---|---|
| | Ag6863, Run |
| Tissue Name | 278700326 |
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 0.0 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 0.0 |
| Ovary | 0.0 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 0.0 |
| Lung | 0.0 |
| Fetal Lung | 0.0 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 0.0 |
| Lung ca. NCI-H526 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 0.0 |
| Liver ca. HepG2 | 0.0 |
| Kidney Pool | 0.0 |
| Fetal Kidney | 0.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 0.0 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.0 |
| Stomach Pool | 0.0 |
| Bone Marrow Pool | 0.0 |
| Fetal Heart | 5.8 |
| Heart Pool | 17.3 |
| Lymph Node Pool | 0.0 |
| Fetal Skeletal Muscle | 100.0 |
| Skeletal Muscle Pool | 0.0 |
| Spleen Pool | 0.0 |
| Thymus Pool | 0.0 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 0.0 |
| Brain (cerebellum) | 0.0 |
| Brain (fetal) | 0.0 |
| Brain (Hippocampus) Pool | 0.0 |
| Cerebral Cortex Pool | 0.0 |
| Brain (Substantia nigra) Pool | 0.0 |
| Brain (Thalamus) Pool | 0.0 |
| Brain (whole) | 0.0 |
| Spinal Cord Pool | 0.0 |
| Adrenal Gland | 0.0 |
| Pituitary gland Pool | 0.0 |
| Salivary Gland | 0.0 |
| Thyroid (female) | 0.0 |
| Pancreatic ca. CAPAN2 | 0.0 |
| Pancreas Pool | 0.0 |

General_screening_panel_v1.6 Summary:

Ag6863 Expression is limited to a sample derived from fetal skeletal muscle (CT=34.7). Interestingly, this gene is expressed at much higher levels in fetal (CT=34.7) when compared to adult skeletal muscle (CT=40). This observation suggests that expression of this gene can be used to distinguish fetal from adult skeletal muscle and other samples in this panel. In addition, the relative overexpression of this gene in fetal skeletal muscle suggests that the protein product may enhance muscular growth or development in the fetus and thus may also act in a regenerative capacity in the adult. Therefore, therapeutic modulation of the protein encoded by this gene could be useful in treatment of muscle related diseases. More specifically, treatment of weak or dystrophic muscle with the protein encoded by this gene could restore muscle mass or function.

E. CG153403-02: Dickkopf Related Protein-4 Precursor.

Expression of gene CG153403-02 was assessed using the primer-probe set Ag7176, described in Table EA. Please note that CG153403-01 represents a full-length physical clone.

TABLE EA

Probe Name Ag7176

| | | | Start | SEQ ID |
|---|---|---|---|---|
| Primers | Sequences | Length | Position | No |
| Forward | 5′-ctctgtgtgaacggacaagag-3′ | 21 | 316 | 295 |
| Probe | TET-5′-ccctggactttgctgtgctcgtc-3′-TAMRA | 23 | 369 | 296 |
| Reverse | 5′-ggactggcttacaaattttcgt-3′ | 22 | 400 | 297 |

CNS_neurodegeneration_v1.0 Summary:

Ag7176 Expression of this gene is low/undetectable in all samples on this panel (CTs>35).

Panel 4.1D Summary:

Ag7176 Expression of this gene is low/undetectable in all samples on this panel (CTs>35).

F. CG157760-02: PLAC1.

Expression of gene CG157760-02 was assessed using the primer-probe set Ag7153, described in Table FA. Please note that CG157760-02 represents a full-length physical clone.

TABLE FA

Probe Name Ag7153

| | | | Start | SEQ ID |
|---|---|---|---|---|
| Primers | Sequences | Length | Position | No |
| Forward | 5′-catcagggccagcaaga-3′ | 17 | 342 | 298 |
| Probe | TET-5′-acacctcgtagcatttctcatccttctgg-3′-TAMRA | 29 | 372 | 299 |
| Reverse | 5′-aggtggacaatcgcagttg-3′ | 19 | 429 | 300 |

CNS_neurodegeneration_v1.0 Summary:

Ag7153 Expression of this gene is low/undetectable in all samples on this panel (CTs>35).

Panel 4.1D Summary:

Ag7153 Expression of this gene is low/undetectable in all samples on this panel (CTs>35).

G. CG158114-01: splice variant of melanoma associated antigen gp100.

Expression of gene CG158114-01 was assessed using the primer-probe set Ag5335, described in Table GA. Results of the RTQ-PCR runs are shown in Tables GB and GC.

TABLE GA

Probe Name Ag5335

| | | | Start | SEQ ID |
|---|---|---|---|---|
| Primers | Sequences | Length | Position | No |
| Forward | 5′-gctacaaagggagccaggt-3′ | 20 | 67 | 301 |
| Probe | TET-5′-acayccagtgtatccccaggaaactga-3′-TAMRA | 27 | 96 | 302 |
| Reverse | 5′-cagggaagatgcaggcat-3′ | 18 | 125 | 303 |

TABLE GB
General_screening_panel_v1.5

| | Rel. Exp. (%) |
|---|---|
| | Ag5335, Run |
| Tissue Name | 237370030 |
| Adipose | 0.0 |
| Melanoma* Hs688(A).T | 0.0 |
| Melanoma* Hs688(B).T | 0.0 |
| Melanoma* M14 | 49.7 |
| Melanoma* LOXIMVI | 1.7 |
| Melanoma* SK-MEL-5 | 100.0 |
| Squamous cell carcinoma SCC-4 | 0.0 |
| Testis Pool | 0.1 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.2 |
| Ovarian ca. OVCAR-5 | 0.2 |
| Ovarian ca. IGROV-1 | 0.0 |
| Ovarian ca. OVCAR-8 | 0.1 |
| Ovary | 0.0 |
| Breast ca. MCF-7 | 0.1 |
| Breast ca. MDA-MB-231 | 0.1 |
| Breast ca. BT 549 | 0.0 |
| Breast ca. T47D | 0.1 |
| Breast ca. MDA-N | 0.4 |
| Breast Pool | 0.0 |
| Trachea | 0.0 |
| Lung | 0.0 |
| Fetal Lung | 0.0 |
| Lung ca. NCI-N417 | 0.0 |
| Lung ca. LX-1 | 0.1 |
| Lung ca. NCI-H146 | 0.0 |
| Lung ca. SHP-77 | 0.1 |
| Lung ca. A549 | 0.1 |
| Lung ca. NCI-H526 | 0.0 |
| Lung ca. NCI-H23 | 0.1 |
| Lung ca. NCI-H460 | 0.1 |
| Lung ca. HOP-62 | 0.0 |
| Lung ca. NCI-H522 | 0.1 |
| Liver | 0.0 |
| Fetal Liver | 0.0 |
| Liver ca. HepG2 | 0.0 |
| Kidney Pool | 0.1 |
| Fetal Kidney | 0.0 |
| Renal ca. 786-0 | 0.0 |
| Renal ca. A498 | 0.0 |
| Renal ca. ACHN | 0.1 |
| Renal ca. UO-31 | 0.1 |
| Renal ca. TK-10 | 0.1 |
| Bladder | 0.0 |
| Gastric ca. (liver met.) NCI-N87 | 0.2 |
| Gastric ca. KATO III | 0.0 |
| Colon ca. SW-948 | 0.0 |
| Colon ca. SW480 | 0.2 |
| Colon ca.* (SW480 met) SW620 | 0.0 |
| Colon ca. HT29 | 0.0 |
| Colon ca. HCT-116 | 0.1 |
| Colon ca. CaCo-2 | 0.2 |
| Colon cancer tissue | 0.1 |
| Colon ca. SW1116 | 0.0 |
| Colon ca. Colo-205 | 0.0 |
| Colon ca. SW-48 | 0.1 |
| Colon Pool | 0.0 |
| Small Intestine Pool | 0.0 |
| Stomach Pool | 0.0 |
| Bone Marrow Pool | 0.0 |
| Fetal Heart | 0.0 |
| Heart Pool | 0.0 |
| Lymph Node Pool | 0.1 |
| Fetal Skeletal Muscle | 0.0 |
| Skeletal Muscle Pool | 0.0 |
| Spleen Pool | 0.0 |
| Thymus Pool | 0.1 |
| CNS cancer (glio/astro) U87-MG | 0.0 |
| CNS cancer (glio/astro) U-118-MG | 0.1 |
| CNS cancer (neuro; met) SK-N-AS | 0.1 |
| CNS cancer (astro) SF-539 | 0.0 |
| CNS cancer (astro) SNB-75 | 0.1 |
| CNS cancer (glio) SNB-19 | 0.0 |
| CNS cancer (glio) SF-295 | 0.1 |
| Brain (Amygdala) Pool | 0.0 |
| Brain (cerebellum) | 0.0 |
| Brain (fetal) | 0.0 |
| Brain (Hippocampus) Pool | 0.0 |
| Cerebral Cortex Pool | 0.0 |
| Brain (Substantia nigra) Pool | 0.0 |
| Brain (Thalamus) Pool | 0.0 |
| Brain (whole) | 0.0 |
| Spinal Cord Pool | 0.1 |
| Adrenal Gland | 0.0 |
| Pituitary gland Pool | 0.0 |
| Salivary Gland | 0.0 |
| Thyroid (female) | 0.0 |
| Pancreatic ca. CAPAN2 | 0.1 |
| Pancreas Pool | 0.1 |

TABLE GC
Panel 4.1D

| | Rel. Exp. (%) |
|---|---|
| | Ag5335, Run |
| Tissue Name | 237371375 |
| Secondary Th1 act | 23.3 |
| Secondary Th2 act | 17.7 |
| Secondary Tr1 act | 5.0 |
| Secondary Th1 rest | 0.0 |
| Secondary Th2 rest | 4.4 |
| Secondary Tr1 rest | 0.0 |
| Primary Th1 act | 0.0 |
| Primary Th2 act | 42.3 |
| Primary Tr1 act | 97.3 |
| Primary Th1 rest | 9.2 |
| Primary Th2 rest | 16.7 |
| Primary Tr1 rest | 6.3 |
| CD45RA CD4 lymphocyte act | 31.9 |
| CD45RO CD4 lymphocyte act | 71.7 |
| CD8 lymphocyte act | 7.9 |
| Secondary CD8 lymphocyte rest | 74.7 |
| Secondary CD8 lymphocyte act | 0.0 |
| CD4 lymphocyte none | 4.6 |
| 2ry Th1/Th2/Tr1_anti-CD95 CH11 | 7.5 |
| LAK cells rest | 21.2 |
| LAK cells IL-2 | 21.0 |
| LAK cells IL-2 + IL-12 | 3.7 |
| LAK cells IL-2 + IFN gamma | 9.9 |
| LAK cells IL-2 + IL-18 | 8.5 |
| LAK cells PMA/ionomycin | 49.0 |
| NK Cells IL-2 rest | 39.0 |
| Two Way MLR 3 day | 5.5 |
| Two Way MLR 5 day | 5.7 |
| Two Way MLR 7 day | 10.8 |
| PBMC rest | 3.9 |
| PBMC PWM | 0.0 |
| PBMC PHA-L | 8.8 |
| Ramos (B cell) none | 0.0 |
| Ramos (B cell) ionomycin | 26.4 |
| B lymphocytes PWM | 5.1 |
| B lymphocytes CD40L and IL-4 | 29.7 |
| EOL-1 dbcAMP | 0.0 |
| EOL-1 dbcAMP PMA/ionomycin | 0.0 |
| Dendritic cells none | 14.7 |
| Dendritic cells LPS | 0.0 |
| Dendritic cells anti-CD40 | 0.0 |
| Monocytes rest | 0.0 |
| Monocytes LPS | 5.8 |
| Macrophages rest | 0.0 |
| Macrophages LPS | 15.3 |
| HUVEC none | 13.2 |
| HUVEC starved | 10.1 |
| HUVEC IL-1beta | 0.0 |
| HUVEC IFN gamma | 7.9 |
| HUVEC TNF alpha + IFN gamma | 0.0 |
| HUVEC TNF alpha + IL4 | 0.0 |
| HUVEC IL-11 | 0.0 |
| Lung Microvascular EC none | 25.0 |
| Lung Microvascular EC TNFalpha + IL-1beta | 0.0 |
| Microvascular Dermal EC none | 0.0 |
| Microsvasular Dermal EC TNFalpha + IL-1beta | 5.6 |
| Bronchial epithelium TNFalpha + IL1beta | 7.4 |
| Small airway epithelium none | 0.0 |
| Small airway epithelium TNFalpha + IL-1beta | 30.8 |
| Coronery artery SMC rest | 8.9 |
| Coronery artery SMC TNFalpha + IL-1beta | 14.7 |
| Astrocytes rest | 7.6 |
| Astrocytes TNFalpha + IL-1beta | 2.0 |
| KU-812 (Basophil) rest | 0.0 |
| KU-812 (Basophil) PMA/ionomycin | 4.0 |
| CCD1106 (Keratinocytes) none | 26.4 |
| CCD1106 (Keratinocytes) TNFalpha + IL-1beta | 16.7 |
| Liver cirrhosis | 2.2 |
| NCI-H292 none | 52.1 |
| NCI-H292 IL-4 | 58.6 |
| NCI-H292 IL-9 | 100.0 |
| NCI-H292 IL-13 | 63.3 |
| NCI-H292 IFN gamma | 8.9 |
| HPAEC none | 3.6 |
| HPAEC TNF alpha + IL-1 beta | 5.8 |
| Lung fibroblast none | 0.0 |
| Lung fibroblast TNF alpha + IL-1 beta | 4.7 |
| Lung fibroblast IL-4 | 1.9 |
| Lung fibroblast IL-9 | 0.0 |
| Lung fibroblast IL-13 | 0.0 |
| Lung fibroblast IFN gamma | 4.4 |
| Dermal fibroblast CCD1070 rest | 5.3 |
| Dermal fibroblast CCD1070 TNF alpha | 18.0 |
| Dermal fibroblast CCD1070 IL-1 beta | 0.0 |
| Dermal fibroblast IFN gamma | 12.2 |
| Dermal fibroblast IL-4 | 17.4 |
| Dermal Fibroblasts rest | 0.0 |
| Neutrophils TNFa + LPS | 0.0 |
| Neutrophils rest | 5.5 |
| Colon | 0.0 |
| Lung | 0.0 |
| Thymus | 7.3 |
| Kidney | 16.2 |

CNS_neurodegeneration_v1.0 Summary:

Ag5335 Expression of this gene is low/undetectable in all samples on this panel (CTs>35).

General_screening_panel_v1.5 Summary:

Ag5335 This gene is very highly expressed in two melanoma cancer cell line samples (CTs=22). This novel gene encodes a protein that is homologous to Melanocyte protein Pmel 17 which plays an important role in melanogenesis and is actively investigated as targets for melanoma immunotherapy (Martinez-Esparza M, Pigment Cell Res 2000 April; 13(2): 120-6). Thus, expression of this gene could be used to differentiate between these samples and other samples on this panel and as a marker to detect the presence of melanoma. Furthermore, therapeutic modulation of the expression or function of this gene may be effective in the treatment of melanoma.

Among tissues with metabolic function, this gene is expressed at low but significant levels in pancreas, and adult and fetal and liver. This expression among these tissues suggests that this gene product may play a role in normal neuroendocrine and metabolic function and that disregulated expression of this gene may contribute to neuroendocrine disorders or metabolic diseases, such as obesity and diabetes.

Panel 4.1D Summary:

Ag5335 Highest expression is seen in a sample derived from IL-9 treated NCI-H292 goblet cells (CT=33.5). Low but significant expression is also seen in NCI-H292 cells treated with IL-4, IL-13, or untreated cells, as well as in PMA/ionomycin treated LAK cells, untreated NK cells, primary activated Th1 and Tr2 cells, CD45RO CD4 lymphocytes and resting secondary CD8 lymphocytes. This expression suggests that this gene product may be involved in inflammatory conditions of the lung, including asthma, emphysema, and allergy.

H. CG158553-01: Erythropoietin Receptor Precursor.

Expression of gene CG158553-01 was assessed using the primer-probe set Ag5446, described in Table HA.

TABLE HA

Probe Name Ag5446

| | | | Start | SEQ ID |
|---|---|---|---|---|
| Primers | Sequences | Length | Position | No |
| Forward | 5′-tcccagggccatgg-3′ | 14 | 1298 | 304 |
| Probe | TET-5′-ccaccccacctaaagtacctgtacctt-3′-TAMRA | 28 | 1339 | 305 |
| Reverse | 5′-agttgagatgccagagtcagat-3′ | 22 | 1371 | 306 |

AI_comprehensive panel_v1.0 Summary:

Ag5446 Expression of this gene is low/undetectable in all samples on this panel (CTs>35).

General_screening_panel_v1.5 Summary:

Ag5446 Expression of this gene is low/undetectable in all samples on this panel (CTs>35).

Panel 4.1D Summary:

Ag5446 Expression of this gene is low/undetectable in all samples on this panel (CTs>35).

I. CG158983-01, CG158983-02 and CG158983-03: Chloride Channel.

Expression of gene CG158983-01, CG158983-02, and CG158983-03 was assessed using the primer-probe sets Ag5892 and Ag6186, described in Tables IA and IB. Results of the RTQ-PCR runs are shown in Tables IC, ID, IE, IF, IG and IH. Please note that CG158983-03 represents a full-length physical clone of the CG158983-02 gene, validating the prediction of the gene sequence.

TABLE IA

Probe Name Ag5892

| | | | Start | SEQ ID |
|---|---|---|---|---|
| Primers | Sequences | Length | Position | No |
| Forward | 5′-agaccaagctccagctgttt-3′ | 20 | 24 | 307 |
| Probe | TET-5′-ctctcccgtcctcactcgcctt-3′-TAMRA | 23 | 47 | 308 |
| Reverse | 5′-aggagcaggaccatgaagag-3′ | 20 | 98 | 309 |

TABLE IB

Probe Name Ag6186

| | | | | |
|---|---|---|---|---|
| Primers | Sequences | Length | Start Position | SEQ ID No |

| | | | | |
|---|---|---|---|---|
| Forward | 5′-ctgcagatcgaggactttctg-3′ | 21 | 242 | 310 |
| Probe | TET-5′-ccgcccgaggagtccaaca-3′-TAMRA | 19 | 278 | 311 |
| Reverse | 5′-gatgaacgcggagaacttgt-3′ | 20 | 318 | 312 |

TABLE IC
AI.05 chondrosarcoma

| | Rel. Exp. (%) |
|---|---|
| | Ag5892, Run |
| Tissue Name | 308433431 |
| 138353_PMA (18 hrs) | 0.0 |
| 138352_IL-1beta + Oncostatin M (18 hrs) | 0.0 |
| 138351_IL-1beta + TNFa (18 hrs) | 9.5 |
| 138350_IL-1beta (18 hrs) | 7.8 |
| 138354_Untreated-complete medium (18 hrs) | 12.0 |
| 138347_PMA (6 hrs) | 23.5 |
| 138346_IL-1beta + Oncostatin M (6 hrs) | 31.6 |
| 138345_IL-1beta + TNFa (6 hrs) | 7.6 |
| 138344_IL-1beta (6 hrs) | 26.8 |
| 138348_Untreated-complete medium (6 hrs) | 56.3 |
| 138349_Untreated-serum starved (6 hrs) | 100.0 |

TABLE ID
AI_comprehensive panel_v1.0

| | Rel. Exp. (%) |
|---|---|
| | Ag5892, Run |
| Tissue Name | 249079679 |
| 110967 COPD-F | 1.1 |
| 110980 COPD-F | 0.8 |
| 110968 COPD-M | 1.5 |
| 110977 COPD-M | 0.7 |
| 110989 Emphysema-F | 1.3 |
| 110992 Emphysema-F | 0.7 |
| 110993 Emphysema-F | 0.3 |
| 110994 Emphysema-F | 0.5 |
| 110995 Emphysema-F | 2.3 |
| 110996 Emphysema-F | 0.5 |
| 110997 Asthma-M | 9.5 |
| 111001 Asthma-F | 0.9 |
| 111002 Asthma-F | 1.4 |
| 111003 Atopic Asthma-F | 2.1 |
| 111004 Atopic Asthma-F | 2.9 |
| 111005 Atopic Asthma-F | 1.4 |
| 111006 Atopic Asthma-F | 0.5 |
| 111417 Allergy-M | 1.1 |
| 112347 Allergy-M | 0.0 |
| 112349 Normal Lung-F | 0.1 |
| 112357 Normal Lung-F | 0.3 |
| 112354 Normal Lung-M | 0.2 |
| 112374 Crohns-F | 0.7 |
| 112389 Match Control Crohns-F | 71.2 |
| 112375 Crohns-F | 1.0 |
| 112732 Match Control Crohns-F | 40.9 |
| 112725 Crohns-M | 0.3 |
| 112387 Match Control Crohns-M | 0.6 |
| 112378 Crohns-M | 0.1 |
| 112390 Match Control Crohns-M | 0.7 |
| 112726 Crohns-M | 3.5 |
| 112731 Match Control Crohns-M | 0.4 |
| 112380 Ulcer Col-F | 0.6 |
| 112734 Match Control Ulcer Col-F | 100.0 |
| 112384 Ulcer Col-F | 1.4 |
| 112737 Match Control Ulcer Col-F | 1.1 |
| 112386 Ulcer Col-F | 1.1 |
| 112738 Match Control Ulcer Col-F | 2.1 |
| 112381 Ulcer Col-M | 0.2 |
| 112735 Match Control Ulcer Col-M | 0.3 |
| 112382 Ulcer Col-M | 35.6 |
| 112394 Match Control Ulcer Col-M | 0.4 |
| 112383 Ulcer Col-M | 1.2 |
| 112736 Match Control Ulcer Col-M | 42.6 |
| 112423 Psoriasis-F | 0.5 |
| 112427 Match Control Psoriasis-F | 0.3 |
| 112418 Psoriasis-M | 0.5 |
| 112723 Match Control Psoriasis-M | 1.1 |
| 112419 Psoriasis-M | 1.2 |
| 112424 Match Control Psoriasis-M | 0.0 |
| 112420 Psoriasis-M | 1.1 |
| 112425 Match Control Psoriasis-M | 0.7 |
| 104689 (MF) OA Bone-Backus | 3.1 |
| 104690 (MF) Adj “Normal” Bone-Backus | 1.4 |
| 104691 (MF) OA Synovium-Backus | 0.7 |
| 104692 (BA) OA Cartilage-Backus | 21.9 |
| 104694 (BA) OA Bone-Backus | 5.2 |
| 104695 (BA) Adj “Normal” Bone-Backus | 1.4 |
| 104696 (BA) OA Synovium-Backus | 0.7 |
| 104700 (SS) OA Bone-Backus | 1.6 |
| 104701 (SS) Adj “Normal” Bone-Backus | 4.2 |
| 104702 (SS) OA Synovium-Backus | 2.1 |
| 117093 OA Cartilage Rep7 | 0.9 |
| 112672 OA Bone5 | 1.0 |
| 112673 OA Synovium5 | 0.4 |
| 112674 OA Synovial Fluid cells5 | 0.3 |
| 117100 OA Cartilage Rep14 | 0.3 |
| 112756 OA Bone9 | 2.0 |
| 112757 OA Synovium9 | 0.2 |
| 112758 OA Synovial Fluid Cells9 | 1.3 |
| 117125 RA Cartilage Rep2 | 1.1 |
| 113492 Bone2 RA | 27.5 |
| 113493 Synovium2 RA | 8.0 |
| 113494 Syn Fluid Cells RA | 18.8 |
| 113499 Cartilage4 RA | 31.6 |
| 113500 Bone4 RA | 37.9 |
| 113501 Synovium4 RA | 25.5 |
| 113502 Syn Fluid Cells4 RA | 17.9 |
| 113495 Cartilage3 RA | 25.9 |
| 113496 Bone3 RA | 27.5 |
| 113497 Synovium3 RA | 16.0 |
| 113498 Syn Fluid Cells3 RA | 30.6 |
| 117106 Normal Cartilage Rep20 | 0.8 |
| 113663 Bone3 Normal | 0.0 |
| 113664 Synovium3 Normal | 0.0 |
| 113665 Syn Fluid Cells3 Normal | 0.0 |
| 117107 Normal Cartilage Rep22 | 0.2 |
| 113667 Bone4 Normal | 0.0 |
| 113668 Synovium4 Normal | 0.2 |
| 113669 Syn Fluid Cells4 Normal | 0.4 |

TABLE IE
General_screening_panel_v1.5

| | Rel. Exp. (%) |
|---|---|
| | Ag5892, Run |
| Tissue Name | 247291076 |
| Adipose | 0.8 |
| Melanoma* Hs688(A).T | 24.8 |
| Melanoma* Hs688(B).T | 20.0 |
| Melanoma* M14 | 0.0 |
| Melanoma* LOXIMVI | 0.2 |
| Melanoma* SK-MEL-5 | 0.0 |
| Squamous cell carcinoma SCC-4 | 1.1 |
| Testis Pool | 1.1 |
| Prostate ca.* (bone met) PC-3 | 3.0 |
| Prostate Pool | 0.6 |
| Placenta | 95.3 |
| Uterus Pool | 1.6 |
| Ovarian ca OVCAR-3 | 0.8 |
| Ovarian ca. SK-OV-3 | 15.4 |
| Ovarian ca. OVCAR-4 | 11.0 |
| Ovarian ca. OVCAR-5 | 30.4 |
| Ovarian ca. IGROV-1 | 0.8 |
| Ovarian ca. OVCAR-8 | 11.7 |
| Ovary | 0.8 |
| Breast ca. MCF-7 | 2.9 |
| Breast ca. MDA-MB-231 | 48.6 |
| Breast ca. BT 549 | 0.1 |
| Breast ca. T47D | 44.4 |
| Breast ca. MDA-N | 0.0 |
| Breast Pool | 0.1 |
| Trachea | 1.1 |
| Lung | 0.0 |
| Fetal Lung | 20.7 |
| Lung ca. NCI-N417 | 0.0 |
| Lung ca LX-1 | 1.9 |
| Lung ca. NCI-H146 | 0.0 |
| Lung ca. SHP-77 | 0.1 |
| Lung ca. A549 | 0.3 |
| Lung ca. NCI-H526 | 0.0 |
| Lung ca NCI-H23 | 0.3 |
| Lung ca. NCI-H460 | 0.0 |
| Lung ca. HOP-62 | 1.3 |
| Lung ca. NCI-H522 | 0.5 |
| Liver | 0.0 |
| Fetal Liver | 0.1 |
| Liver ca. HepG2 | 1.0 |
| Kidney Pool | 0.3 |
| Fetal Kidney | 0.1 |
| Renal ca. 786-0 | 3.3 |
| Renal ca. A498 | 0.0 |
| Renal ca. ACHN | 0.3 |
| Renal ca. UO-31 | 0.6 |
| Renal ca. TK-10 | 1.3 |
| Bladder | 0.4 |
| Gastric ca. (liver met.) NCI-N87 | 100.0 |
| Gastric ca. KATO III | 1.1 |
| Colon ca. SW-948 | 1.9 |
| Colon ca. SW480 | 3.8 |
| Colon ca.* (SW480 met) SW620 | 0.8 |
| Colon ca. HT29 | 8.7 |
| Colon ca. HCT-116 | 1.6 |
| Colon ca. CaCo-2 | 4.4 |
| Colon cancer tissue | 1.5 |
| Colon ca. SW1116 | 1.2 |
| Colon ca. Colo-205 | 0.1 |
| Colon ca. SW-48 | 0.0 |
| Colon Pool | 0.3 |
| Small Intestine Pool | 0.1 |
| Stomach Pool | 0.1 |
| Bone Marrow Pool | 1.3 |
| Fetal Heart | 0.1 |
| Heart Pool | 0.2 |
| Lymph Node Pool | 0.2 |
| Fetal Skeletal Muscle | 0.1 |
| Skeletal Muscle Pool | 0.3 |
| Spleen Pool | 0.6 |
| Thymus Pool | 1.0 |
| CNS cancer (glio/astro) U87-MG | 0.1 |
| CNS cancer (glio/astro) U-118-MG | 0.1 |
| CNS cancer (neuro; met) SK-N-AS | 0.1 |
| CNS cancer (astro) SF-539 | 0.3 |
| CNS cancer (astro) SNB-75 | 5.1 |
| CNS cancer (glio) SNB-19 | 1.2 |
| CNS cancer (glio) SF-295 | 0.0 |
| Brain (Amygdala) Pool | 0.0 |
| Brain (cerebellum) | 0.1 |
| Brain (fetal) | 0.1 |
| Brain (Hippocampus) Pool | 0.1 |
| Cerebral Cortex Pool | 0.1 |
| Brain (Substantia nigra) Pool | 0.1 |
| Brain (Thalamus) Pool | 0.0 |
| Brain (whole) | 0.1 |
| Spinal Cord Pool | 0.3 |
| Adrenal Gland | 0.1 |
| Pituitary gland Pool | 0.1 |
| Salivary Gland | 0.5 |
| Thyroid (female) | 67.4 |
| Pancreatic ca. CAPAN2 | 6.0 |
| Pancreas Pool | 0.1 |

TABLE IF
Panel 4.1D

| | Rel. Exp. (%) |
|---|---|
| | Ag5892, Run |
| Tissue Name | 247290537 |
| Secondary Th1 act | 0.3 |
| Secondary Th2 act | 0.0 |
| Secondary Tr1 act | 0.0 |
| Secondary Th1 rest | 0.2 |
| Secondary Th2 rest | 0.2 |
| Secondary Tr1 rest | 0.0 |
| Primary Th1 act | 0.0 |
| Primary Th2 act | 0.0 |
| Primary Tr1 act | 0.0 |
| Primary Th1 rest | 0.3 |
| Primary Th2 rest | 2.4 |
| Primary Tr1 rest | 0.0 |
| CD45RA CD4 lymphocyte act | 0.2 |
| CD45RO CD4 lymphocyte act | 2.2 |
| CD8 lymphocyte act | 0.2 |
| Secondary CD8 lymphocyte rest | 0.6 |
| Secondary CD8 lymphocyte act | 0.0 |
| CD4 lymphocyte none | 0.2 |
| 2ry Th1/Th2/Tr1_anti-CD95 CH11 | 0.8 |
| LAK cells rest | 2.3 |
| LAK cells IL-2 | 11.2 |
| LAK cells IL-2 + IL-12 | 3.6 |
| LAK cells IL-2 + IFN gamma | 3.2 |
| LAK cells IL-2 + IL-18 | 1.5 |
| LAK cells PMA/ionomycin | 2.3 |
| NK Cells IL-2 rest | 31.0 |
| Two Way MLR 3 day | 2.5 |
| Two Way MLR 5 day | 0.2 |
| Two Way MLR 7 day | 1.2 |
| PBMC rest | 2.3 |
| PBMC PWM | 1.1 |
| PBMC PHA-L | 0.5 |
| Ramos (B cell) none | 0.0 |
| Ramos (B cell) ionomycin | 0.0 |
| B lymphocytes PWM | 0.2 |
| B lymphocytes CD40L and IL-4 | 0.5 |
| EOL-1 dbcAMP | 0.0 |
| EOL-1 dbcAMP PMA/ionomycin | 0.0 |
| Dendritic cells none | 0.5 |
| Dendritic cells LPS | 0.5 |
| Dendritic cells anti-CD40 | 0.0 |
| Monocytes rest | 0.0 |
| Monocytes LPS | 2.2 |
| Macrophages rest | 0.2 |
| Macrophages LPS | 0.5 |
| HUVEC none | 0.0 |
| HUVEC starved | 1.2 |
| HUVEC IL-1beta | 0.9 |
| HUVEC. IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 7.0 |
| Lung Microvascular EC TNFalpha + IL-1beta | 0.5 |
| Microvascular Dermal EC none | 0.0 |
| Microsvasular Dermal EC TNFalpha + IL-1beta | 1.0 |
| Bronchial epithelium TNFalpha + IL1beta | 8.2 |
| Small airway epithelium none | 100.0 |
| Small airway epithelium TNFalpha + IL-1beta | 77.9 |
| Coronery artery SMC rest | 2.1 |
| Coronery artery SMC TNFalpha + IL-1beta | 0.8 |
| Astrocytes rest | 0.6 |
| Astrocytes TNFalpha + IL-1beta | 2.2 |
| KU-812 (Basophil) rest | 0.5 |
| KU-812 (Basophil) PMA/ionomycin | 0.0 |
| CCD1106 (Keratinocytes) none | 8.8 |
| CCD1106 (Keratinocytes) TNFalpha + IL-1beta | 7.1 |
| Liver cirrhosis | 0.0 |
| NCI-H292 none | 10.7 |
| NCI-H292 IL-4 | 6.3 |
| NCI-H292 IL-9 | 10.4 |
| NCI-H292 IL-13 | 4.2 |
| NCI-H292 IFN gamma | 4.6 |
| HPAEC none | 0.0 |
| HPAEC TNF alpha + IL-1 beta | 0.2 |
| Lung fibroblast none | 1.9 |
| Lung fibroblast TNF alpha + IL-1 beta | 0.9 |
| Lung fibroblast IL-4 | 2.4 |
| Lung fibroblast IL-9 | 7.8 |
| Lung fibroblast IL-13 | 0.0 |
| Lung fibroblast IFN gamma | 4.8 |
| Dermal fibroblast CCD1070 rest | 6.4 |
| Dermal fibroblast CCD1070 TNF alpha | 4.3 |
| Dermal fibroblast CCD1070 IL-1 beta | 2.3 |
| Dermal fibroblast IFN gamma | 2.9 |
| Dermal fibroblast IL-4 | 1.1 |
| Dermal Fibroblasts rest | 1.2 |
| Neutrophils TNFa + LPS | 0.0 |
| Neutrophils rest | 0.0 |
| Colon | 0.0 |
| Lung | 4.5 |
| Thymus | 0.1 |
| Kidney | 0.2 |

TABLE IG
Panel 5 Islet

| | Rel. Exp. (%) |
|---|---|
| | Ag5892, Run |
| Tissue Name | 253578281 |
| 97457_Patient-02go_adipose | 3.2 |
| 97476_Patient-07sk_skeletal muscle | 0.3 |
| 97477_Patient-07ut_uterus | 0.1 |
| 97478_Patient-07pl_placenta | 40.1 |
| 99167_Bayer Patient 1 | 0.6 |
| 97482_Patient-08ut_uterus | 0.2 |
| 97483_Patient-08pl_placenta | 47.3 |
| 97486_Patient-09sk_skeletal muscle | 0.0 |
| 97487_Patient-09ut_uterus | 0.1 |
| 97488_Patient-09pl_placenta | 33.2 |
| 97492_Patient-10ut_uterus | 0.0 |
| 97493_Patient-10pl_placenta | 62.4 |
| 97495_Patient-11go_adipose | 4.1 |
| 97496_Patient-11sk_skeletal muscle | 0.2 |
| 97497_Patient-11ut_uterus | 0.4 |
| 97498_Patient-11pl_placenta | 47.6 |
| 97500_Patient-12go_adipose | 3.3 |
| 97501_Patient-12sk_skeletal muscle | 0.0 |
| 97502_Patient-12ut_uterus | 0.6 |
| 97503_Patient-12pl_placenta | 100.0 |
| 94721_Donor 2 U - A_Mesenchymal Stem Cells | 0.9 |
| 94722_Donor 2 U - B_Mesenchymal Stem Cells | 2.0 |
| 94723_Donor 2 U - C_Mesenchymal Stem Cells | 1.9 |
| 94709_Donor 2 AM - A_adipose | 0.3 |
| 94710_Donor 2 AM - B_adipose | 0.9 |
| 94711_Donor 2 AM - C_adipose | 0.5 |
| 94712_Donor 2 AD - A_adipose | 0.6 |
| 94713_Donor 2 AD - B_adipose | 0.7 |
| 94714_Donor 2 AD - C_adipose | 0.6 |
| 94742_Donor 3 U - A_Mesenchymal Stem Cells | 2.3 |
| 94743_Donor 3 U - B_Mesenchymal Stem Cells | 9.5 |
| 94730_Donor 3 AM - A_adipose | 2.7 |
| 94731_Donor 3 AM - B_adipose | 1.8 |
| 94732_Donor 3 AM - C_adipose | 1.2 |
| 94733_Donor 3 AD - A_adipose | 6.3 |
| 94734_Donor 3 AD - B_adipose | 1.6 |
| 94735_Donor 3 AD - C_adipose | 11.9 |
| 77138_Liver_HepG2untreated | 2.3 |
| 73556_Heart_Cardiac stromal cells (primary) | 0.1 |
| 81735_Small Intestine | 0.4 |
| 72409_Kidney_Proximal Convoluted Tubule | 0.0 |
| 82685_Small intestine_Duodenum | 0.0 |
| 90650_Adrenal_Adrenocortical adenoma | 0.0 |
| 72410_Kidney_HRCE | 3.7 |
| 72411_Kidney_HRE | 0.8 |
| 73139_Uterus_Uterine smooth muscle cells | 0.7 |

TABLE IH
general oncology screening panel_v_2.4

| | Rel. Exp. (%) |
|---|---|
| | Ag5892, Run |
| Tissue Name | 260316169 |
| Colon cancer 1 | 3.7 |
| Colon NAT 1 | 2.5 |
| Colon cancer 2 | 25.7 |
| Colon NAT 2 | 4.6 |
| Colon cancer 3 | 19.2 |
| Colon NAT 3 | 13.9 |
| Colon malignant cancer 4 | 12.5 |
| Colon NAT 4 | 2.1 |
| Lung cancer 1 | 29.7 |
| Lung NAT 1 | 18.9 |
| Lung cancer 2 | 36.6 |
| Lung NAT 2 | 17.0 |
| Squamous cell carcinoma 3 | 62.0 |
| Lung NAT 3 | 10.1 |
| Metastatic melanoma 1 | 2.7 |
| Melanoma 2 | 13.9 |
| Melanoma 3 | 42.9 |
| Metastatic melanoma 4 | 14.2 |
| Metastatic melanoma 5 | 8.8 |
| Bladder cancer 1 | 0.0 |
| Bladder NAT 1 | 0.0 |
| Bladder cancer 2 | 3.5 |
| Bladder NAT 2 | 0.0 |
| Bladder NAT 3 | 1.1 |
| Bladder NAT 4 | 2.3 |
| Prostate adenocarcinoma 1 | 4.8 |
| Prostate adenocarcinoma 2 | 0.8 |
| Prostate adenocarcinoma 3 | 5.1 |
| Prostate adenocarcinoma 4 | 100.0 |
| Prostate NAT 5 | 2.8 |
| Prostate adenocarcinoma 6 | 4.4 |
| Prostate adenocarcinoma 7 | 4.1 |
| Prostate adenocarcinoma 8 | 2.6 |
| Prostate adenocarcinoma 9 | 5.8 |
| Prostate NAT 10 | 2.2 |
| Kidney cancer 1 | 3.9 |
| Kidney NAT 1 | 1.8 |
| Kidney cancer 2 | 20.4 |
| Kidney NAT 2 | 2.4 |
| Kidney cancer 3 | 2.6 |
| Kidney NAT 3 | 0.2 |
| Kidney cancer 4 | 1.5 |
| Kidney NAT 4 | 6.6 |

AI.05 Chondrosarcoma Summary:

Ag5892 Highest expression of this gene is detected in untreated serum starved chondrosarcoma cell line (SW1353) (CT=32.2). Interestingly, expression of this gene appears to be slightly down regulated upon treatment with IL-1 (CTs=334-35), a potent activator of pro-inflammatory cytokines and matrix metalloproteinases that participate in the destruction of cartilage observed in osteoarthritis (OA). Modulation of the expression of this transcript in chondrocytes may therefore be important for preventing the degeneration of cartilage observed in OA.

AI_Comprehensive Panel_v1.0 Summary:

Ag5892 Highest expression is seen in a sample derived from normal tissue adjacent to ulcerative colitis (CT=27.7). In addition, prominent levels of expression are seen in a cluster of samples derived from rheumatoid arthritis, as well as in an OA sample. Thus, expression of this gene could be used to differentiate these samples from other samples and as a marker of these diseases. Furthermore, therapeutic modulation of the expression or function of this gene may be useful in the treatment of these diseases. Ag5892 Results from a second experiment with this probe and primer, run 247842321, are not included. The amp plot indicates that there were experimental difficulties with this run. Ag6186 Expression of this gene is low/undetectable in all samples on this panel (CTs>35).

General_screening_panel_v1.5 Summary:

Ag5892 Highest expression is seen in a gastric cancer cell line (CT=28). Moderate levels of expression are also seen in a cluster of cell lines derived from breast, ovarian, and melanoma cancers, as well as in normal thyroid, fetal lung and placenta. In addition, this gene is expressed at much higher levels in fetal lung tissue (CT=30) when compared to expression in the adult counterpart (CT=40). Thus, expression of this gene may be used to differentiate between the fetal and adult source of this tissue.

Panel 4.1D Summary:

Ag5892 Prominent expression of this gene is seen in untreated small airway epithelium, as well as in small airway epithelium treated with TNF-a and IL-1 b (CTs=28-29). In addition, low but significant levels of expression are seen in clusters of samples derived from lung and dermal fibroblasts, as well as in NCI-H292 goblet cells. Thus, expression of this gene could be used as as a marker of small airway epithelium. Furthermore, modulation of the expression or function of this gene may be useful in the treatment of inflammatory conditions of the lung, including allergy, emphysema, and asthma. Ag6186 Expression of this gene is low/undetectable in all samples on this panel (CTs>35).

Panel 5 Islet Summary:

Ag5892 Expression of this gene is prominent in placenta, consistent with expression in Panel 1.5 (CTs=28-30). Thus, expression of this gene could be used as a marker of this tissue.

General Oncology Screening Panel_V_{—}2.4 Summary:

Ag5892 Highest expression of this gene is seen in a prostate cancer (CT=30). Moderate levels of expression of this gene are seen in colon, lung, kidney, melanoma, and skin cell carcinoma cancers. Thus, expression of this gene may be useful of a marker of these or other cancers, particularly hormone dependent cancers like breast cancers. In addition, modulation of the expression or function of this gene may be useful in the treatment of cancers.

J. CG159015-01, CG159015-02, and CG159015-03: Novel Secreted Protein.

Expression of gene CG159015-01, CG159015-02, and CG159015-03 was assessed using the primer-probe set Ag5962, described in Table JA. Results of the RTQ-PCR runs are shown in Tables JB and JC. Please note that CG159015-03 represents a full-length physical clone.

TABLE JA

Probe Name Ag5962

| | | | | |
|---|---|---|---|---|
| Primers | Sequences | Length | Start Position | SEQ ID No |

| | | | | |
|---|---|---|---|---|
| Forward | 5′-aaagatgaaactgcggt-3′ | 17 | 338 | 313 |
| Probe | TET-5-tccacgaggaggcaagcaa-3′-TAMRA | 19 | 357 | 314 |
| Reverse | 5′-agctgttgctctgact-3′ | 16 | 390 | 315 |

TABLE JB
General_screening_panel_v1.5

| | Rel. Exp. (%) |
|---|---|
| | Ag5962, Run |
| Tissue Name | 248162755 |
| Adipose | 1.6 |
| Melanoma* Hs688(A).T | 21.5 |
| Melanoma* Hs688(B).T | 20.2 |
| Melanoma* M14 | 14.5 |
| Melanoma* LOXIMVI | 10.7 |
| Melanoma* SK-MEL-5 | 14.1 |
| Squamous cell carcinoma SCC-4 | 2.6 |
| Testis Pool | 4.2 |
| Prostate ca.* (bone met) PC-3 | 7.3 |
| Prostate Pool | 4.4 |
| Placenta | 2.3 |
| Uterus Pool | 1.3 |
| Ovarian ca. OVCAR-3 | 10.0 |
| Ovarian ca. SK-OV-3 | 30.1 |
| Ovarian ca. OVCAR-4 | 9.0 |
| Ovarian ca. OVCAR-5 | 17.8 |
| Ovarian ca. IGROV-1 | 26.8 |
| Ovarian ca. OVCAR-8 | 16.8 |
| Ovary | 5.2 |
| Breast ca. MCF-7 | 10.7 |
| Breast ca. MDA-MB-231 | 29.3 |
| Breast ca. BT 549 | 34.9 |
| Breast ca. T47D | 2.9 |
| Breast ca. MDA-N | 3.6 |
| Breast Pool | 11.7 |
| Trachea | 4.9 |
| Lung | 3.3 |
| Fetal Lung | 5.6 |
| Lung ca. NCI-N417 | 1.6 |
| Lung ca. LX-1 | 9.7 |
| Lung ca. NCI-H146 | 3.7 |
| Lung ca. SHP-77 | 3.1 |
| Lung ca. A549 | 9.9 |
| Lung ca. NCI-H526 | 3.8 |
| Lung ca. NCI-H23 | 12.8 |
| Lung ca. NCI-H460 | 6.5 |
| Lung ca. HOP-62 | 6.7 |
| Lung ca. NCI-H522 | 8.8 |
| Liver | 1.0 |
| Fetal Liver | 2.6 |
| Liver ca. HepG2 | 8.2 |
| Kidney Pool | 22.7 |
| Fetal Kidney | 2.8 |
| Renal ca. 786-0 | 7.7 |
| Renal ca. A498 | 10.5 |
| Renal ca. ACHN | 6.0 |
| Renal ca. UO-31 | 5.8 |
| Renal ca. TK-10 | 6.5 |
| Bladder | 5.5 |
| Gastric ca. (liver met.) NCI-N87 | 6.6 |
| Gastric ca. KATO III | 6.7 |
| Colon ca. SW-948 | 6.6 |
| Colon ca. SW480 | 12.7 |
| Colon ca.* (SW480 met) SW620 | 8.4 |
| Colon ca. HT29 | 9.3 |
| Colon ca. HCT-116 | 12.8 |
| Colon ca. CaCo-2 | 6.5 |
| Colon cancer tissue | 10.8 |
| Colon ca. SW1116 | 3.4 |
| Colon ca. Colo-205 | 2.0 |
| Colon ca. SW-48 | 1.6 |
| Colon Pool | 13.4 |
| Small Intestine Pool | 6.3 |
| Stomach Pool | 6.9 |
| Bone Marrow Pool | 1.7 |
| Fetal Heart | 3.1 |
| Heart Pool | 12.7 |
| Lymph Node Pool | 15.9 |
| Fetal Skeletal Muscle | 2.8 |
| Skeletal Muscle Pool | 25.5 |
| Spleen Pool | 6.5 |
| Thymus Pool | 9.0 |
| CNS cancer (glio/astro) U87-MG | 49.7 |
| CNS cancer (glio/astro) U-118-MG | 27.0 |
| CNS cancer (neuro; met) SK-N-AS | 7.5 |
| CNS cancer (astro) SF-539 | 7.9 |
| CNS cancer (astro) SNB-75 | 100.0 |
| CNS cancer (glio) SNB-19 | 25.9 |
| CNS cancer (glio) SF-295 | 26.8 |
| Brain (Amygdala) Pool | 5.8 |
| Brain (cerebellum) | 26.6 |
| Brain (fetal) | 6.5 |
| Brain (Hippocampus) Pool | 5.3 |
| Cerebral Cortex Pool | 6.1 |
| Brain (Substantia nigra) Pool | 6.7 |
| Brain (Thalamus) Pool | 6.7 |
| Brain (whole) | 3.3 |
| Spinal Cord Pool | 5.8 |
| Adrenal Gland | 5.8 |
| Pituitary gland Pool | 3.2 |
| Salivary Gland | 2.9 |
| Thyroid (female) | 4.7 |
| Pancreatic ca. CAPAN2 | 3.1 |
| Pancreas Pool | 13.6 |

TABLE JC
Panel 5 Islet

| | Rel. Exp. (%) |
|---|---|
| | Ag5962, Run |
| Tissue Name | 248195280 |
| 97457_Patient-02go adipose | 20.2 |
| 97476_Patient-07sk_skeletal muscle | 15.9 |
| 97477_Patient-07ut_uterus | 20.2 |
| 97478_Patient-07pl_placenta | 8.9 |
| 99167_Bayer Patient 1 | 100.0 |
| 97482_Patient-08ut_uterus | 17.4 |
| 97483_Patient-08pl_placenta | 4.7 |
| 97486_Patient-09sk_skeletal muscle | 10.0 |
| 97487_Patient-09ut_uterus | 49.0 |
| 97488_Patient-09pl_placenta | 4.4 |
| 97492_Patient-10ut_uterus | 32.3 |
| 97493_Patient-10pl_placenta | 7.5 |
| 97495_Patient-11go_adipose | 5.2 |
| 97496_Patient-11sk_skeletal muscle | 12.0 |
| 97497_Patient-11ut_uterus | 34.9 |
| 97498_Patient-11pl_placenta | 3.5 |
| 97500_Patient-12go_adipose | 11.1 |
| 97501_Patient-12sk_skeletal muscle | 25.7 |
| 97502_Patient-12ut_uterus | 46.3 |
| 97503_Patient-12pl_placenta | 3.4 |
| 94721_Donor 2 U - A_Mesenchymal Stem Cells | 15.0 |
| 94722_Donor 2 U - B_Mesenchymal Stem Cells | 8.2 |
| 94723_Donor 2 U - C_Mesenchymal Stem Cells | 12.4 |
| 94709_Donor 2 AM - A_adipose | 21.3 |
| 94710_Donor 2 AM - B_adipose | 14.0 |
| 94711_Donor 2 AM - C_adipose | 9.7 |
| 94712_Donor 2 AD - A_adipose | 20.4 |
| 94713_Donor 2 AD - B_adipose | 18.4 |
| 94714_Donor 2 AD - C_adipose | 21.5 |
| 94742_Donor 3 U - A_Mesenchymal Stem Cells | 5.4 |
| 94743_Donor 3 U - B_Mesenchymal Stem Cells | 10.6 |
| 94730_Donor 3 AM - A_adipose | 31.2 |
| 94731_Donor 3 AM - B_adipose | 11.7 |
| 94732_Donor 3 AM - C_adipose | 8.7 |
| 94733_Donor 3 AD - A_adipose | 22.1 |
| 94734_Donor 3 AD - B_adipose | 4.3 |
| 94735_Donor 3 AD - C_adipose | 13.4 |
| 77138_Liver HepG2untreated | 16.7 |
| 73556_Heart_Cardiac stromal cells (primary) | 5.0 |
| 81735_Small Intestine | 18.7 |
| 72409_Kidney_Proximal Convoluted Tubule | 3.3 |
| 82685_Small intestine_Duodenum | 0.6 |
| 90650_Adrenal_Adrenocortical adenoma | 23.7 |
| 72410_Kidney_HRCE | 23.5 |
| 72411_Kidney_HRE | 14.2 |
| 73139_Uterus_Uterine smooth muscle cells | 10.1 |

General_screening_panel_v1.5 Summary:

Ag5962 Highest expression of this gene is detected in brain cancer SNB-75 cell line (CT=25.2). Moderate to high levels of expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Panel 5 Islet Summary:

Ag5962 Highest expression of this gene is detected in islet cells (CT=27.7). This gene shows wide spread expression in this panel, with moderate expressions in adipose, skeletal muscle, uterus, placenta, small intestine, cardiac stromal cells and kidney. Therefore, therapeutic modulation of this gene may be useful in the treatment of endocrine/metabolically related diseases, such as obesity and diabetes, including type II diabetes.

K. CG50387-03: Connexin 46.

Expression of gene CG50387-03 was assessed using the primer-probe sets Ag2597, Ag5234 and Ag5235, described in Tables KA, KB and KC. Results of the RTQ-PCR runs are shown in Tables KD and KE.

TABLE KA

Probe Name Ag2597

| | | | Start | SEQ ID |
|---|---|---|---|---|
| Primers | Sequences | Length | Position | No |
| Forward | 5′-ggagctttctgggaaqactct-3′ | 21 | 11 | 316 |
| Probe | TET-5′-tagaaaatgcacaggagcactccacg-3′-TAMRA | 26 | 32 | 317 |
| Reverse | 5′-caaaatgcggaagatgaaca-3′ | 20 | 86 | 318 |

TABLE KB

Probe Name Ag5234

| | | | Start | SEQ ID |
|---|---|---|---|---|
| Primers | Sequences | Length | Position | No |
| Forward | 5′-cttcatcatcttcatgctggcg-3′ | 22 | 606 | 319 |
| Probe | TET-5′-cactgctgctcaacatgctggagatata-3′-TAMRA | 28 | 641 | 320 |
| Reverse | 5′-ggctggtcacgccctgctt-3′ | 19 | 691 | 321 |

TABLE KC

Probe Name Ag5235

| | | | Start | SEQ ID |
|---|---|---|---|---|
| Primers | Sequences | Length | Position | No |
| Forward | 5′-gcggacttcaaaatgctagccctgacc-3′ | 27 | 883 | 322 |
| Probe | TET-5′-ccagtccgccaagctctacaacgg-3′-TAMRA | 24 | 927 | 323 |
| Reverse | 5′-gcccagttctgctcagtcatcagc-3′ | 24 | 963 | 324 |

TABLE KD
General_screening_panel_v1.5

| | Rel. | Rel. |
|---|---|---|
| | Exp. (%) | Exp. (%) |
| | Ag5234, Run | Ag5235, Run |
| Tissue Name | 229514466 | 229514467 |
| Adipose | 0.0 | 0.3 |
| Melanoma* Hs688(A).T | 1.4 | 0.3 |
| Melanoma* Hs688(B).T | 0.8 | 0.0 |
| Melanoma* M14 | 40.1 | 41.2 |
| Melanoma* LOXIMVI | 2.0 | 7.6 |
| Melanoma* SK-MEL-5 | 32.1 | 22.5 |
| Squamous cell carcinoma SCC-4 | 3.4 | 2.3 |
| Testis Pool | 1.6 | 2.7 |
| Prostate ca.* (bone met) PC-3 | 3.5 | 0.0 |
| Prostate Pool | 0.0 | 0.0 |
| Placenta | 1.2 | 0.8 |
| Uterus Pool | 0.3 | 0.0 |
| Ovarian ca. OVCAR-3 | 0.8 | 0.0 |
| Ovarian ca. SK-OV-3 | 52.5 | 57.4 |
| Ovarian ca. OVCAR-4 | 6.5 | 6.4 |
| Ovarian ca. OVCAR-5 | 15.2 | 14.3 |
| Ovarian ca. IGROV-1 | 4.9 | 4.7 |
| Ovarian ca. OVCAR-8 | 12.9 | 10.8 |
| Ovary | 0.0 | 1.2 |
| Breast ca. MCF-7 | 0.0 | 1.5 |
| Breast ca. MDA-MB-231 | 42.9 | 31.6 |
| Breast ca. BT 549 | 0.9 | 0.0 |
| Breast ca. T47D | 1.0 | 0.0 |
| Breast ca. MDA-N | 0.0 | 0.0 |
| Breast Pool | 3.8 | 4.7 |
| Trachea | 0.7 | 0.0 |
| Lung | 0.0 | 0.0 |
| Fetal Lung | 0.0 | 0.9 |
| Lung ca. NCI-N417 | 0.5 | 0.0 |
| Lung ca. LX-1 | 0.6 | 0.0 |
| Lung ca. NCI-H146 | 0.0 | 0.0 |
| Lung ca. SHP-77 | 0.0 | 0.0 |
| Lung ca. A549 | 0.7 | 0.0 |
| Lung ca. NCI-H526 | 0.0 | 0.0 |
| Lung ca. NCI-H23 | 12.7 | 11.8 |
| Lung ca. NCI-H460 | 1.6 | 2.7 |
| Lung ca. HOP-62 | 3.6 | 0.6 |
| Lung ca. NCI-H522 | 16.3 | 10.7 |
| Liver | 0.0 | 0.0 |
| Fetal Liver | 0.7 | 0.0 |
| Liver ca. HepG2 | 0.6 | 0.0 |
| Kidney Pool | 1.5 | 3.5 |
| Fetal Kidney | 6.8 | 6.2 |
| Renal ca. 786-0 | 0.6 | 0.0 |
| Renal ca. A498 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Bladder | 1.8 | 1.6 |
| Gastric ca. (liver met.) NCI-N87 | 6.3 | 7.5 |
| Gastric ca. KATO III | 0.7 | 0.0 |
| Colon ca. SW-948 | 0.0 | 0.0 |
| Colon ca. SW480 | 100.0 | 100.0 |
| Colon ca.* (SW480 met) SW620 | 0.0 | 0.6 |
| Colon ca. HT29 | 0.0 | 0.0 |
| Colon ca. HCT-116 | 67.8 | 55.1 |
| Colon ca. CaCo-2 | 0.0 | 0.7 |
| Colon cancer tissue | 0.0 | 0.0 |
| Colon ca. SW1116 | 10.9 | 14.9 |
| Colon ca. Colo-205 | 0.0 | 0.0 |
| Colon ca. SW-48 | 0.0 | 0.0 |
| Colon Pool | 3.6 | 3.6 |
| Small Intestine Pool | 2.1 | 5.3 |
| Stomach Pool | 0.8 | 0.7 |
| Bone Marrow Pool | 0.0 | 0.3 |
| Fetal Heart | 61.1 | 45.4 |
| Heart Pool | 5.8 | 7.2 |
| Lymph Node Pool | 0.1 | 0.0 |
| Fetal Skeletal Muscle | 0.4 | 0.9 |
| Skeletal Muscle Pool | 0.0 | 0.0 |
| Spleen Pool | 0.0 | 0.6 |
| Thymus Pool | 0.5 | 0.2 |
| CNS cancer (glio/astro) U87-MG | 0.0 | 0.0 |
| CNS cancer (glio/astro) U-118-MG | 1.3 | 0.0 |
| CNS cancer (neuro; met) SK-N-AS | 0.0 | 0.0 |
| CNS cancer (astro) SF-539 | 0.4 | 0.0 |
| CNS cancer (astro) SNB-75 | 0.5 | 1.1 |
| CNS cancer (glio) SNB-19 | 3.4 | 5.6 |
| CNS cancer (glio) SF-295 | 4.3 | 5.9 |
| Brain (Amygdala) Pool | 0.3 | 0.0 |
| Brain (cerebellum) | 0.0 | 0.7 |
| Brain (fetal) | 0.0 | 0.0 |
| Brain (Hippocampus) Pool | 0.2 | 0.0 |
| Cerebral Cortex Pool | 0.0 | 0.0 |
| Brain (Substantia nigra) Pool | 0.0 | 1.1 |
| Brain (Thalamus) Pool | 0.0 | 0.0 |
| Brain (whole) | 0.0 | 0.0 |
| Spinal Cord Pool | 0.0 | 1.0 |
| Adrenal Gland | 0.0 | 0.0 |
| Pituitary gland Pool | 0.0 | 0.2 |
| Salivary Gland | 0.0 | 0.0 |
| Thyroid (female) | 0.0 | 0.0 |
| Pancreatic ca. CAPAN2 | 26.2 | 22.5 |
| Pancreas Pool | 3.5 | 0.5 |

TABLE KE
Panel 4.1D

| | Rel. | Rel. |
|---|---|---|
| | Exp. (%) | Exp. (%) |
| | Ag5234, | Ag5235, |
| | Run | Run |
| Tissue Name | 229788208 | 229788230 |
| Secondary Th1 act | 2.6 | 0.0 |
| Secondary Th2 act | 0.0 | 0.0 |
| Secondary Tr1 act | 0.0 | 0.0 |
| Secondary Th1 rest | 0.0 | 0.0 |
| Secondary Th2 rest | 0.0 | 0.0 |
| Secondary Tr1 rest | 0.0 | 0.0 |
| Primary Th1 act | 0.0 | 0.0 |
| Primary Th2 act | 0.0 | 0.0 |
| Primary Tr1 act | 0.0 | 0.0 |
| Primary Th1 rest | 0.0 | 0.0 |
| Primary Th2 rest | 0.0 | 0.0 |
| Primary Tr1 rest | 0.0 | 0.0 |
| CD45RA CD4 lymphocyte act | 0.0 | 0.0 |
| CD45RO CD4 lymphocyte act | 0.0 | 2.7 |
| CD8 lymphocyte act | 0.0 | 0.0 |
| Secondary CD8 lymphocyte rest | 1.6 | 0.0 |
| Secondary CD8 lymphocyte act | 0.0 | 0.0 |
| CD4 lymphocyte none | 1.6 | 0.0 |
| 2ry Th1/Th2/Tr1_anti-CD95 CH11 | 0.0 | 0.0 |
| LAK cells rest | 0.0 | 0.0 |
| LAK cells IL-2 | 0.0 | 2.4 |
| LAK cells IL-2 + IL-12 | 1.1 | 0.0 |
| LAK cells IL-2 + IFN gamma | 0.0 | 0.0 |
| LAK cells IL-2 + IL-18 | 0.0 | 0.0 |
| LAK cells PMA/ionomycin | 2.5 | 2.4 |
| NK Cells IL-2 rest | 0.0 | 0.0 |
| Two Way MLR 3 day | 1.5 | 0.0 |
| Two Way MLR 5 day | 0.0 | 0.0 |
| Two Way MLR 7 day | 0.0 | 0.0 |
| PBMC rest | 0.0 | 0.0 |
| PBMC PWM | 1.2 | 4.5 |
| PBMC PHA-L | 8.4 | 3.0 |
| Ramos (B cell) none | 0.0 | 0.0 |
| Ramos (B cell) ionomycin | 0.0 | 0.0 |
| B lymphocytes PWM | 0.0 | 0.0 |
| B lymphocytes CD40L and IL-4 | 0.0 | 0.0 |
| EOL-1 dbcAMP | 0.0 | 0.0 |
| EOL-1 dbcAMP PMA/ionomycin | 0.0 | 0.0 |
| Dendritic cells none | 0.0 | 0.0 |
| Dendritic cells LPS | 0.0 | 0.0 |
| Dendritic cells anti-CD40 | 0.0 | 0.0 |
| Monocytes rest | 0.0 | 0.0 |
| Monocytes LPS | 100.0 | 100.0 |
| Macrophages rest | 0.0 | 0.0 |
| Macrophages LPS | 0.0 | 0.0 |
| HUVEC none | 0.0 | 0.0 |
| HUVEC starved | 0.0 | 0.0 |
| HUVEC IL-1beta | 0.0 | 0.0 |
| HUVEC IFN gamma | 0.0 | 0.0 |
| HUVEC TNF alpha + IFN gamma | 0.0 | 0.0 |
| HUVEC TNF alpha + IL4 | 0.0 | 0.0 |
| HUVEC IL-11 | 0.0 | 0.0 |
| Lung Microvascular EC none | 0.0 | 0.0 |
| Lung Microvascular EC | 0.0 | 0.0 |
| TNFalpha + IL-1beta | | |
| Microvascular Dermal EC none | 0.0 | 0.0 |
| Microsvasular Dermal EC | 0.0 | 0.0 |
| TNFalpha + IL-1beta | | |
| Bronchial epithelium | 0.0 | 1.7 |
| TNFalpha + IL1beta | | |
| Small airway epithelium none | 0.0 | 0.0 |
| Small airway epithelium | 8.4 | 7.7 |
| TNFalpha + IL-1beta | | |
| Coronery artery SMC rest | 0.0 | 0.0 |
| Coronery artery SMC | 0.0 | 0.0 |
| TNFalpha + IL-1beta | | |
| Astrocytes rest | 4.8 | 3.9 |
| Astrocytes | 6.5 | 0.0 |
| TNFalpha + IL-1beta | | |
| KU-812 (Basophil) rest | 0.0 | 0.0 |
| KU-812 (Basophil) PMA/ionomycin | 0.0 | 0.0 |
| CCD1106 (Keratinocytes) none | 9.2 | 6.8 |
| CCD1106 (Keratinocytes) | 0.0 | 3.3 |
| TNFalpha + IL-1beta | | |
| Liver cirrhosis | 0.0 | 0.0 |
| NCI-H292 none | 0.0 | 0.0 |
| NCI-H292 IL-4 | 0.0 | 0.0 |
| NCI-H292 IL-9 | 0.0 | 2.3 |
| NCI-H292 IL-13 | 2.7 | 4.1 |
| NCI-H292 IFN gamma | 0.0 | 0.0 |
| HPAEC none | 0.0 | 0.0 |
| HPAEC TNF alpha + IL-1 beta | 0.0 | 0.0 |
| Lung fibroblast none | 8.7 | 1.6 |
| Lung fibroblast | 17.0 | 20.3 |
| TNF alpha + IL-1 beta | | |
| Lung fibroblast IL-4 | 2.1 | 0.0 |
| Lung fibroblast IL-9 | 6.1 | 4.0 |
| Lung fibroblast IL-13 | 0.0 | 0.0 |
| Lung fibroblast IFN gamma | 6.3 | 3.2 |
| Dermal fibroblast CCD1070 rest | 0.0 | 0.0 |
| Dermal fibroblast CCD1070 TNF | 3.4 | 0.0 |
| alpha | | |
| Dermal fibroblast | 3.9 | 0.0 |
| CCD1070 IL-1beta | | |
| Dermal fibroblast IFN gamma | 8.6 | 0.0 |
| Dermal fibroblast IL-4 | 3.2 | 7.5 |
| Dermal Fibroblasts rest | 1.8 | 3.3 |
| Neutrophils TNFa + LPS | 0.0 | 0.0 |
| Neutrophils rest | 0.0 | 0.0 |
| Colon | 0.0 | 0.0 |
| Lung | 0.0 | 0.0 |
| Thymus | 0.0 | 0.0 |
| Kidney | 2.4 | 3.6 |

CNS_neurodegeneration_v1.0 Summary:

Ag2597/Ag5234/Ag5235 Expression of this gene is low/undetectable in all samples on this panel (CTs>35).

General_screening_panel_v1.5 Summary:

Ag5234/Ag5235 Two experiments with two different probe-primer sets are in excellent agreement. Highest expression of this gene is detected in a sample derived from a colon cancer cell line (SW480) (CTs=30). In addition, there is substantial expression associated with two other colon cancer cell lines, a pancreatic cancer cell line, two lung cancer cell lines, a breast cancer cell line, two melanoma cell lines and a cluster of several ovarian cancer cell lines. Thus, the expression of this gene could be used to distinguish the above samples from the other samples in the panel. Moreover, therapeutic modulation of this gene, through the use of small molecule drugs, antibodies or protein therapeutics might be of benefit in the treatment of ovarian, colon, pancreatic, lung, breast cancers or melanoma.

This gene is also expressed at moderate levels in fetal heart (CT=31.1) and at lower levels in the adult heart (CT=34.5). Thus, expression of this gene may be used to differentiate between fetal and adult heart tissue. Furthermore, the higher levels of expression in fetal heart suggest that the protein encoded by this gene may be important for the pathogenesis, diagnosis, and/or treatment of diseases of the heart.

Panel 4.1D Summary:

Ag5234/Ag5235 Two experiments with two different prob-primer sets are in excellent agreement. Highest expression of this gene is detected mainly in monocytes stimulated with LPS (CTs=32). Upon activation with pathogens, including bacterial LPS, monocytes contribute to the innate and specific immunity by migrating to the site of tissue injury and releasing inflammatory cytokines. This release contributes to the inflammation process. This transcript encodes for a connexin like protein, a family of proteins that is involved in gap junction and intercellular communication. Thus, the protein encoded by this transcript may play a role in the interaction of activated monocytes with the endothelium. This is the first step necessary for the migration of these cells to injured tissue. Therefore, modulation of the expression or the function of the protein encoded by this gene, by antibodies or small molecules can prevent the recruitment of monocytes and the inflammatory process, and lead to improvement of the symptoms of patients suffering from autoimmune and inflammatory diseases such as asthma, allergies, inflammatory bowel disease, lupus erythematosus, or rheumatoid arthritis.

L. CG52113-01, CG52113-03, CG52113-04, CG52113-05, and CG52113-06: Notch 4 Like Protein.

Expression of gene CG52113-01 was assessed using the primer-probe sets Ag2665 and Ag2778, described in Tables LA and LB. Results of the RTQ-PCR runs are shown in Tables LC, LD, LE and LF. Please note that CG52113-05 represents a full-length physical clone of the CG52113-01 gene, validating the prediction of the gene sequence.

TABLE LA

Probe Name Ag2665

| | | | Start | SEQ ID |
|---|---|---|---|---|
| Primers | Sequences | Length | Position | No |
| Forward | 5′-gtctgcagacggtacactctgt-3′ | 22 | 602 | 325 |
| Probe | TET-5′-cccaacccgacaggagtggacag-3′-TAMRA | 23 | 654 | 326 |
| Reverse | 5′-gcacttgttccttcattgca-3′ | 20 | 677 | 327 |

TABLE LB

Probe Name Ag2778

| | | | Start | SEQ ID |
|---|---|---|---|---|
| Primers | Sequences | Length | Position | No |
| Forward | 5′-gtctgcagacggtacactctgt-3′ | 22 | 602 | 328 |
| Probe | TET-5′-cccaacccgacaggagtggacag-3′-TAMRA | 23 | 654 | 329 |
| Reverse | 5′-gcacttcttccttcattgca-3′ | 20 | 677 | 330 |

TABLE LC
CNS_neurodegeneration_v1.0

| | Rel. Exp. (%) | Rel. Exp. (%) | Rel. Exp. (%) | Rel. Exp. (%) |
|---|---|---|---|---|
| | Ag2665, Run | Ag2665, Run | Ag2778, Run | Ag2778, Run |
| Tissue Name | 206955568 | 230512508 | 208699215 | 269216134 |
| AD 1 Hippo | 20.9 | 19.8 | 30.1 | 10.8 |
| AD 2 Hippo | 42.6 | 27.5 | 46.7 | 22.5 |
| AD 3 Hippo | 10.4 | 15.0 | 19.2 | 5.0 |
| AD 4 Hippo | 11.8 | 16.0 | 22.7 | 5.1 |
| AD 5 Hippo | 84.7 | 70.7 | 98.6 | 29.9 |
| AD 6 Hippo | 37.9 | 45.7 | 54.0 | 19.8 |
| Control 2 Hippo | 28.7 | 25.5 | 28.3 | 12.9 |
| Control 4 Hippo | 9.3 | 8.9 | 24.5 | 8.1 |
| Control (Path) 3 Hippo | 16.0 | 9.9 | 17.2 | 6.0 |
| AD 1 Temporal Ctx | 19.3 | 15.8 | 10.7 | 7.2 |
| AD 2 Temporal Ctx | 45.4 | 44.8 | 68.8 | 14.4 |
| AD 3 Temporal Ctx | 14.4 | 13.0 | 10.3 | 5.6 |
| AD 4 Temporal Ctx | 30.8 | 39.5 | 33.0 | 21.2 |
| AD 5 Inf Temporal Ctx | 100.0 | 87.1 | 100.0 | 28.1 |
| AD 5 Sup Temporal Ctx | 42.3 | 40.9 | 70.2 | 21.0 |
| AD 6 Inf Temporal Ctx | 55.5 | 49.7 | 39.5 | 22.4 |
| AD 6 Sup Temporal Ctx | 46.0 | 50.0 | 82.4 | 28.5 |
| Control 1 Temporal Ctx | 23.0 | 17.7 | 25.7 | 9.3 |
| Control 2 Temporal Ctx | 57.0 | 61.6 | 42.3 | 23.8 |
| Control 3 Temporal Ctx | 42.6 | 40.3 | 42.6 | 22.1 |
| Control 3 Temporal Ctx | 39.2 | 27.0 | 26.1 | 8.3 |
| Control (Path) 1 Temporal Ctx | 68.3 | 73.7 | 85.9 | 34.9 |
| Control (Path) 2 Temporal Ctx | 78.5 | 59.9 | 54.7 | 34.9 |
| Control (Path) 3 Temporal Ctx | 23.0 | 21.8 | 19.8 | 9.5 |
| Control (Path) 4 Temporal Ctx | 82.4 | 64.2 | 62.9 | 28.3 |
| AD 1 Occipital Ctx | 10.9 | 20.3 | 19.6 | 9.0 |
| AD 2 Occipital Ctx (Missing) | 0.0 | 0.0 | 2.0 | 0.0 |
| AD 3 Occipital Ctx | 19.6 | 10.5 | 25.7 | 8.1 |
| AD 4 Occipital Ctx | 39.2 | 37.1 | 33.2 | 12.2 |
| AD 5 Occipital Ctx | 43.2 | 39.0 | 48.3 | 8.5 |
| AD 6 Occipital Ctx | 25.5 | 22.8 | 32.8 | 18.8 |
| Control 1 Occipital Ctx | 18.3 | 8.7 | 23.7 | 10.7 |
| Control 2 Occipital Ctx | 81.8 | 81.2 | 78.5 | 29.5 |
| Control 3 Occipital Ctx | 42.9 | 38.4 | 59.0 | 19.8 |
| Control 4 Occipital Ctx | 16.4 | 16.8 | 13.3 | 7.2 |
| Control (Path) 1 Occipital Ctx | 52.1 | 71.7 | 75.8 | 25.2 |
| Control (Path) 2 Occipital Ctx | 37.4 | 39.8 | 27.2 | 16.2 |
| Control (Path) 3 Occipital Ctx | 25.5 | 17.4 | 18.8 | 100.0 |
| Control (Path) 4 Occipital Ctx | 60. 7 | 33.7 | 57.8 | 24.5 |
| Control 1 Parietal Ctx | 33.4 | 25.5 | 37.9 | 12.3 |
| Control 2 Parietal Ctx | 42.0 | 59.9 | 47.3 | 27.2 |
| Control 3 Parietal Ctx | 39.8 | 27.7 | 28.3 | 17.0 |
| Control (Path) 1 Parietal Ctx | 88.3 | 100.0 | 87.1 | 37.9 |
| Control (Path) 2 Parietal Ctx | 57.4 | 54.0 | 59.9 | 23.5 |
| Control (Path) 3 Parietal Ctx | 15.2 | 18.4 | 31.2 | 8.6 |
| Control (Path) 4 Parietal Ctx | 82.9 | 56.6 | 52.9 | 28.9 |

TABLE LD
Panel 1.3D

| | Rel. | Rel. |
|---|---|---|
| | Exp. (%) | Exp. (%) |
| | Ag2665, | Ag2778, |
| | Run | Run |
| Tissue Name | 160075204 | 164023413 |
| Liver adenocarcinoma | 5.0 | 17.3 |
| Pancreas | 3.0 | 2.3 |
| Pancreatic ca. CAPAN 2 | 1.2 | 1.2 |
| Adrenal gland | 4.7 | 2.5 |
| Thyroid | 10.4 | 15.0 |
| Salivary gland | 3.3 | 2.5 |
| Pituitary gland | 3.1 | 4.3 |
| Brain (fetal) | 1.4 | 1.1 |
| Brain (whole) | 10.3 | 12.4 |
| Brain (amygdala) | 12.7 | 14.4 |
| Brain (cerebellum) | 0.2 | 0.9 |
| Brain (hippocampus) | 100.0 | 33.2 |
| Brain (substantia nigra) | 3.7 | 4.8 |
| Brain (thalamus) | 17.9 | 18.2 |
| Cerebral Cortex | 13.9 | 22.8 |
| Spinal cord | 2.8 | 10.3 |
| glio/astro U87-MG | 0.0 | 0.0 |
| glio/astro U-118-MG | 0.0 | 0.0 |
| astrocytoma SW1783 | 1.7 | 3.7 |
| neuro*; met SK-N-AS | 0.0 | 0.0 |
| astrocytoma SF-539 | 0.2 | 1.0 |
| astrocytoma SNB-75 | 3.8 | 1.5 |
| glioma SNB-19 | 3.1 | 7.2 |
| glioma U251 | 1.3 | 0.5 |
| glioma SF-295 | 3.9 | 4.4 |
| Heart (fetal) | 36.9 | 96.6 |
| Heart | 6.3 | 21.6 |
| Skeletal muscle (fetal) | 41.5 | 100.0 |
| Skeletal muscle | 2.1 | 12.9 |
| Bone marrow | 4.4 | 3.3 |
| Thymus | 2.5 | 19.5 |
| Spleen | 28.5 | 43.8 |
| Lymph node | 6.0 | 8.2 |
| Colorectal | 0.8 | 1.0 |
| Stomach | 3.8 | 3.6 |
| Small intestine | 11.4 | 12.8 |
| Colon ca. SW480 | 6.0 | 4.2 |
| Colon ca.* SW620 (SW480 met) | 0.0 | 0.3 |
| Colon ca. HT29 | 0.0 | 0.8 |
| Colon ca. HCT-116 | 3.5 | 6.6 |
| Colon ca. CaCo-2 | 2.0 | 6.7 |
| Colon ca. tissue (ODO3866) | 1.7 | 6.0 |
| Colon ca. HCC-2998 | 7.9 | 2.2 |
| Gastric ca.* (liver met) | 2.7 | 2.2 |
| NCI-N87 | | |
| Bladder | 2.0 | 4.5 |
| Trachea | 16.6 | 19.1 |
| Kidney | 3.7 | 16.7 |
| Kidney (fetal) | 14.4 | 26.2 |
| Renal ca. 786-0 | 2.6 | 2.9 |
| Renal ca. A498 | 16.6 | 10.0 |
| Renal ca. RXF 393 | 1.0 | 3.0 |
| Renal ca. ACHN | 2.6 | 4.2 |
| Renal ca. UO-31 | 1.7 | 2.3 |
| Renal ca. TK-10 | 0.0 | 0.0 |
| Liver | 3.3 | 4.2 |
| Liver (fetal) | 21.8 | 20.6 |
| Liver ca. (hepatoblast) HepG2 | 0.3 | 1.4 |
| Lung | 57.4 | 47.0 |
| Lung (fetal) | 25.7 | 57.4 |
| Lung ca. (small cell) LX-1 | 0.5 | 1.1 |
| Lung ca. (small cell) NCI-H69 | 2.0 | 0.5 |
| Lung ca. (s. cell var.) SHP-77 | 0.9 | 0.3 |
| Lung ca. (large cell) NCI-H460 | 6.8 | 8.4 |
| Lung ca. (non-sm. cell) A549 | 4.1 | 2.1 |
| Lung ca. (non-s. cell) NCI-H23 | 8.5 | 6.1 |
| Lung ca. (non-s. cell) HOP-62 | 3.1 | 4.2 |
| Lung ca. (non-s. cl) NCI-H522 | 2.2 | 2.0 |
| Lung ca. (squam.) SW 900 | 0.5 | 0.8 |
| Lung ca. (squam.) NCI-H596 | 0.0 | 0.0 |
| Mammary gland | 17.9 | 19.3 |
| Breast ca.* (pl. ef) MCF-7 | 3.0 | 4.2 |
| Breast ca.* (pl. ef) MDA-MB-231 | 10.7 | 5.1 |
| Breast ca.* (pl. ef) T47D | 1.8 | 1.2 |
| Breast ca. BT-549 | 1.5 | 0.9 |
| Breast ca. MDA-N | 0.0 | 0.0 |
| Ovary | 5.9 | 8.1 |
| Ovarian ca. OVCAR-3 | 3.8 | 6.8 |
| Ovarian ca. OVCAR-4 | 1.0 | 1.4 |
| Ovarian ca. OVCAR-5 | 5.0 | 6.3 |
| Ovarian ca. OVCAR-8 | 2.3 | 4.9 |
| Ovarian ca. IGROV-1 | 1.8 | 1.7 |
| Ovarian ca.* (ascites) SK-OV-3 | 2.4 | 1.7 |
| Uterus | 11.7 | 14.9 |
| Placenta | 30.1 | 27.4 |
| Prostate | 7.4 | 9.2 |
| Prostate ca.* (bone met) PC-3 | 1.0 | 0.4 |
| Testis | 19.3 | 34.2 |
| Melanoma Hs688(A).T | 1.2 | 0.0 |
| Melanoma* (met) Hs688(B).T | 0.3 | 1.0 |
| Melanoma UACC-62 | 2.2 | 4.8 |
| Melanoma M14 | 1.3 | 0.9 |
| Melanoma LOX IMVI | 5.6 | 3.4 |
| Melanoma* (met) SK-MEL-5 | 2.8 | 1.8 |
| Adipose | 3.0 | 6.6 |

TABLE LE
Panel 2D

| | Rel. | Rel. |
|---|---|---|
| | Exp. (%) | Exp. (%) |
| | Ag2665, Run | Ag2778, Run |
| Tissue Name | 160093572 | 162440337 |
| Normal Colon | 7.9 | 9.2 |
| CC Well to Mod Diff (ODO3866) | 14.5 | 15.1 |
| CC Margin (ODO3866) | 5.9 | 6.5 |
| CC Gr.2 rectosigmoid (ODO3868) | 3.2 | 3.9 |
| CC Margin (ODO3868) | 4.0 | 4.0 |
| CC Mod Diff (ODO3920) | 4.3 | 4.5 |
| CC Margin (ODO3920) | 5.1 | 6.4 |
| CC Gr.2 ascend colon (ODO3921) | 8.5 | 10.2 |
| CC Margin (ODO3921) | 7.7 | 5.7 |
| CC from Partial Hepatectomy | 31.9 | 32.8 |
| (ODO4309) Mets | | |
| Liver Margin (ODO4309) | 14.1 | 15.9 |
| Colon mets to lung (OD04451-01) | 14.6 | 19.1 |
| Lung Margin (OD04451-02) | 19.9 | 26.6 |
| Normal Prostate 6546-1 | 10.4 | 50.0 |
| Prostate Cancer (OD04410) | 16.5 | 18.8 |
| Prostate Margin (OD04410) | 14.9 | 16.3 |
| Prostate Cancer (OD04720-01) | 7.5 | 10.3 |
| Prostate Margin (OD04720-02) | 18.3 | 23.5 |
| Normal Lung 061010 | 58.6 | 46.7 |
| Lung Met to Muscle (ODO4286) | 8.1 | 9.9 |
| Muscle Margin (ODO4286) | 22.1 | 25.9 |
| Lung Malignant Cancer (OD03126) | 19.3 | 28.9 |
| Lung Margin (OD03126) | 100.0 | 87.7 |
| Lung Cancer (OD04404) | 15.1 | 14.0 |
| Lung Margin (OD04404) | 53.6 | 47.0 |
| Lung Cancer (OD04565) | 2.5 | 5.8 |
| Lung Margin (OD04565) | 41.8 | 66.9 |
| Lung Cancer (OD04237-01) | 4.4 | 7.3 |
| Lung Margin (OD04237-02) | 51.8 | 68.3 |
| Ocular Mel Met to Liver | 10.7 | 16.8 |
| (ODO4310) | | |
| Liver Margin (ODO4310) | 11.4 | 16.3 |
| Melanoma Mets to Lung (OD04321) | 13.0 | 17.2 |
| Lung Margin (OD04321) | 89.5 | 95.3 |
| Normal Kidney | 14.9 | 24.5 |
| Kidney Ca, Nuclear grade 2 | 7.2 | 7.8 |
| (OD04338) | | |
| Kidney Margin (OD04338) | 29.5 | 20.2 |
| Kidney Ca Nuclear grade 1/2 | 2.8 | 3.9 |
| (OD04339) | | |
| Kidney Margin (OD04339) | 18.6 | 25.0 |
| Kidney Ca, Clear cell | 52.9 | 70.2 |
| type (OD04340) | | |
| Kidney Margin (OD04340) | 21.6 | 26.4 |
| Kidney Ca, Nuclear grade 3 | 20.6 | 30.1 |
| (OD04348) | | |
| Kidney Margin (OD04348) | 13.9 | 29.9 |
| Kidney Cancer (OD04622-01) | 18.4 | 18.7 |
| Kidney Margin (OD04622-03) | 12.1 | 9.7 |
| Kidney Cancer (OD04450-01) | 0.4 | 2.7 |
| Kidney Margin (OD04450-03) | 9.7 | 22.7 |
| Kidney Cancer 8120607 | 7.3 | 9.9 |
| Kidney Margin 8120608 | 19.2 | 22.1 |
| Kidney Cancer 8120613 | 7.8 | 11.5 |
| Kidney Margin 8120614 | 17.8 | 23.8 |
| Kidney Cancer 9010320 | 18.6 | 20.2 |
| Kidney Margin 9010321 | 32.5 | 29.9 |
| Normal Uterus | 24.8 | 25.9 |
| Uterus Cancer 064011 | 27.5 | 31.6 |
| Normal Thyroid | 14.8 | 13.8 |
| Thyroid Cancer 064010 | 7.2 | 9.4 |
| Thyroid Cancer A302152 | 5.7 | 8.2 |
| Thyroid Margin A302153 | 18.4 | 27.9 |
| Normal Breast | 25.7 | 47.6 |
| Breast Cancer (OD04566) | 8.4 | 6.3 |
| Breast Cancer (OD04590-01) | 27.4 | 27.9 |
| Breast Cancer Mets (OD04590-03) | 47.3 | 100.0 |
| Breast Cancer Metastasis | 11.6 | 12.0 |
| (OD04655-05) | | |
| Breast Cancer 064006 | 4.3 | 7.8 |
| Breast Cancer 1024 | 13.9 | 13.9 |
| Breast Cancer 9100266 | 9.9 | 15.5 |
| Breast Margin 9100265 | 3.0 | 7.7 |
| Breast Cancer A209073 | 8.0 | 10.4 |
| Breast Margin A209073 | 7.9 | 7.3 |
| Normal Liver | 3.2 | 6.2 |
| Liver Cancer 064003 | 2.2 | 1.1 |
| Liver Cancer 1025 | 7.4 | 5.4 |
| Liver Cancer 1026 | 16.8 | 17.8 |
| Liver Cancer 6004-T | 8.4 | 6.9 |
| Liver Tissue 6004-N | 2.3 | 1.6 |
| Liver Cancer 6005-T | 23.7 | 25.3 |
| Liver Tissue 6005-N | 7.9 | 10.4 |
| Normal Bladder | 15.4 | 19.5 |
| Bladder Cancer 1023 | 2.5 | 2.5 |
| Bladder Cancer A302173 | 0.9 | 0.3 |
| Bladder Cancer (OD04718-01) | 2.6 | 2.8 |
| Bladder Normal Adjacent | 19.1 | 29.5 |
| (OD04718-03) | | |
| Normal Ovary | 15.1 | 23.5 |
| Ovarian Cancer 064008 | 8.3 | 11.4 |
| Ovarian Cancer (OD04768-07) | 3.9 | 2.7 |
| Ovary Margin (OD04768-08) | 25.5 | 20.6 |
| Normal Stomach | 5.5 | 6.7 |
| Gastric Cancer 9060358 | 1.3 | 0.9 |
| Stomach Margin 9060359 | 3.0 | 3.2 |
| Gastric Cancer 9060395 | 9.8 | 13.1 |
| Stomach Margin 9060394 | 7.4 | 7.7 |
| Gastric Cancer 9060397 | 28.9 | 26.2 |
| Stomach Margin 9060396 | 3.3 | 3.1 |
| Gastric Cancer 064005 | 5.0 | 5.6 |

TABLE LF
Panel 4D

| | Rel. | Rel. |
|---|---|---|
| | Exp. (%) | Exp. (%) |
| | Ag2665, | Ag2778, |
| | Run | Run |
| Tissue Name | 158912341 | 161930458 |
| Secondary Th1 act | 0.1 | 0.0 |
| Secondary Th2 act | 0.2 | 0.2 |
| Secondary Tr1 act | 0.0 | 1.2 |
| Secondary Th1 rest | 0.0 | 0.0 |
| Secondary Th2 rest | 0.2 | 0.0 |
| Secondary Tr1 rest | 0.0 | 0.0 |
| Primary Th1 act | 0.0 | 0.7 |
| Primary Th2 act | 0.5 | 0.3 |
| Primary Tr1 act | 0.2 | 0.0 |
| Primary Th1 rest | 0.2 | 0.0 |
| Primary Th2 rest | 0.0 | 0.0 |
| Primary Tr1 rest | 0.0 | 0.0 |
| CD45RA CD4 lymphocyte act | 1.5 | 0.9 |
| CD45RO CD4 lymphocyte act | 0.1 | 0.2 |
| CD8 lymphocyte act | 0.2 | 0.0 |
| Secondary CD8 lymphocyte rest | 0.6 | 0.3 |
| Secondary CD8 lymphocyte act | 0.0 | 0.0 |
| CD4 lymphocyte none | 0.0 | 0.0 |
| 2ry Th1/Th2/Tr1_anti-CD95 CH11 | 0.0 | 0.0 |
| LAK cells rest | 0.0 | 0.0 |
| LAK cells IL-2 | 0.1 | 0.1 |
| LAK cells IL-2 + IL-12 | 0.0 | 0.0 |
| LAK cells IL-2 + IFN gamma | 0.2 | 0.0 |
| LAK cells IL-2 + IL-18 | 0.0 | 0.0 |
| LAK cells PMA/ionomycin | 0.0 | 0.4 |
| NK Cells IL-2 rest | 0.0 | 0.4 |
| Two Way MLR 3 day | 0.1 | 0.2 |
| Two Way MLR 5 day | 0.0 | 0.1 |
| Two Way MLR 7 day | 0.0 | 0.2 |
| PBMC rest | 0.0 | 0.5 |
| PBMC PWM | 0.1 | 0.0 |
| PBMC PHA-L | 0.1 | 0.0 |
| Ramos (B cell) none | 0.0 | 0.0 |
| Ramos (B cell) ionomycin | 0.0 | 0.0 |
| B lymphocytes PWM | 0.1 | 0.3 |
| B lymphocytes CD40L and IL-4 | 0.0 | 0.0 |
| EOL-1 dbcAMP | 4.5 | 4.8 |
| EOL-1 dbcAMP PMA/ionomycin | 2.0 | 0.8 |
| Dendritic cells none | 2.1 | 1.0 |
| Dendritic cells LPS | 1.4 | 2.4 |
| Dendritic cells anti-CD40 | 2.0 | 2.0 |
| Monocytes rest | 0.1 | 0.5 |
| Monocytes LPS | 0.0 | 0.2 |
| Macrophages rest | 0.9 | 0.9 |
| Macrophages LPS | 0.0 | 0.4 |
| HUVEC none | 84.1 | 71.2 |
| HUVEC starved | 48.3 | 56.6 |
| HUVEC IL-1beta | 21.6 | 15.7 |
| HUVEC IFN gamma | 64.6 | 49.7 |
| HUVEC TNF alpha + IFN gamma | 15.3 | 18.7 |
| HUVEC TNF alpha + IL4 | 19.9 | 16.2 |
| HUVEC IL-11 | 53.2 | 48.6 |
| Lung Microvascular EC none | 100.0 | 100.0 |
| Lung Microvascular EC | 27.2 | 25.3 |
| TNFalpha + IL-1beta | | |
| Microvascular Dermal EC none | 68.3 | 67.8 |
| Microsvasular Dermal EC | 25.2 | 25.9 |
| TNFalpha + IL-1beta | | |
| Bronchial epithelium | 0.2 | 5.9 |
| TNFalpha + IL1beta | | |
| Small airway epithelium none | 1.4 | 1.5 |
| Small airway epithelium | 2.0 | 4.8 |
| TNFalpha + IL-1beta | | |
| Coronery artery SMC rest | 18.2 | 14.6 |
| Coronery artery SMC | 17.4 | 10.1 |
| TNFalpha + IL-1beta | | |
| Astrocytes rest | 1.7 | 1.0 |
| Astrocytes | 0.8 | 2.5 |
| TNFalpha + IL-1beta | | |
| KU-812 (Basophil) rest | 62.0 | 50.0 |
| KU-812 (Basophil) | 25.5 | 21.6 |
| PMA/ionomycin | | |
| CCD1106 (Keratinocytes) none | 3.2 | 2.8 |
| CCD1106 (Keratinocytes) | 0.3 | 2.8 |
| TNFalpha + IL-1beta | | |
| Liver cirrhosis | 1.7 | 1.9 |
| Lupus kidney | 1.1 | 1.2 |
| NCI-H292 none | 3.2 | 3.2 |
| NCI-H292 IL-4 | 3.0 | 2.2 |
| NCI-H292 IL-9 | 5.3 | 1.9 |
| NCI-H292 IL-13 | 2.3 | 3.0 |
| NCI-H292 IFN gamma | 1.8 | 3.2 |
| HPAEC none | 0.0 | 50.3 |
| HPAEC TNF alpha + IL-1 beta | 21.9 | 34.4 |
| Lung fibroblast none | 1.1 | 1.3 |
| Lung fibroblast | 1.8 | 1.7 |
| TNF alpha + IL-1 beta | | |
| Lung fibroblast IL-4 | 0.4 | 1.3 |
| Lung fibroblast IL-9 | 1.3 | 1.8 |
| Lung fibroblast IL-13 | 2.2 | 0.5 |
| Lung fibroblast IFN gamma | 0.9 | 0.8 |
| Dermal fibroblast CCD1070 rest | 2.0 | 0.9 |
| Dermal fibroblast | 2.4 | 1.0 |
| CCD1070 TNF alpha | | |
| Dermal fibroblast | 1.5 | 0.4 |
| CCD1070 IL-1 beta | | |
| Dermal fibroblast IFN gamma | 0.9 | 0.6 |
| Dermal fibroblast IL-4 | 1.0 | 0.6 |
| IBD Colitis 2 | 0.0 | 0.0 |
| IBD Crohn's | 0.3 | 0.3 |
| Colon | 5.0 | 3.0 |
| Lung | 8.2 | 6.7 |
| Thymus | 3.7 | 3.1 |
| Kidney | 3.0 | 1.6 |

CNS_neurodegeneration_v1.0 Summary:

Ag2665/Ag2778 Four experiments with two different probe-primer sets are in good agreement. This panel confirms the expression of this gene at low levels in the brain in an independent group of individuals. This gene is found to be slightly down-regulated in the temporal cortex of Alzheimer's disease patients. Therefore, up-regulation of this gene or its protein product, or treatment with specific agonists for this receptor may be of use in reversing the dementia, memory loss, and neuronal death associated with this disease.

Panel 1.3D Summary:

Ag2665/Ag2778 Two experiments with two different probe-primer sets are in good agreement. Highest expression of this gene is detected in hippocampus and fetal skeletal muscle (CTs=26-28.7). This gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Moderate levels of expression of this gene is also seen in cluster of cancer cell lines derived from pancreatic, gastric, colon, lung, liver, renal, breast, ovarian, prostate, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

Panel 2D Summary:

Ag2665/Ag2778 Two experiments with two different probe-primer sets are in good agreement. Highest expression of this gene is detected in normal lung and a metastatic breast cancer sample (CTs=27-28). This gene show significant expression in both cancer and normal tissue samples derived from colon, ovary, bladder, prostate, liver, breast, thyroid, uterus, kidney and lung. Moderate levels of expression of this gene is also seen in metastatic melanoma. Interestingly, higher expression of this gene is consistently associated with normal lung as compared to corresponding cancer sample. Therefore, expression of this gene may be used to distinguish between cancer and normal lung. Furthermore, therapeutic modulation of this gene or its protein product may be useful in the treatment of metastatic melanoma, colon, ovary, bladder, prostate, liver, breast, thyroid, uterus, kidney and lung cancers

Panel 4D Summary:

Ag2665/Ag2778 Two experiments with two different probe-primer sets are in good agreement. Highest expression of this gene in lung microvascular endothelial cells (CTs=27-28). Moderate to high levels of expression of this gene is mainly seen in endothelial cells. IL-1 beta and TNFalpha treatment reduce the expression of this gene consistently in endothelium samples including HPAEC, HUVEC and lung microvascular EC. Therefore, therapies designed with the protein encoded by this gene may be important in regulating endothelium function including leukocyte extravasation, a major component of inflammation during asthma, IBD, and psoriasis.

In addition, moderate to low levels of expression of this gene is also seen in eosinophils, dendritic cells, resting macrophage, activated CD45RA CD4 lymphocyte, lung and dermal fibroblasts and normal tissues represent by colon, lung, thymus and kidney. Therefore, therapeutic modulation of this gene product may ameliorate symptoms/conditions associated with autoimmune and inflammatory disorders including psoriasis, allergy, asthma, inflammatory bowel disease, rheumatoid arthritis and osteoarthritis

M. CG57542-01: Cadherin.

Expression of gene CG57542-01 was assessed using the primer-probe sets Ag3234, Ag3279 and Ag616, described in Tables MA, MB and MC. Results of the RTQ-PCR runs are shown in Tables MD, ME, MF, MG, MH, MI, MJ, MK and ML.

TABLE MA

Probe Name g3234

| | | | Start | SEQ ID |
|---|---|---|---|---|
| Primers | Sequences | Length | Position | No |
| Forward | 5′-gcaaaatcgtcgtctctgttac-3′ | 22 | 668 | 331 |
| Probe | TET-5′-ccctctgaaagccaccagcagtg-3′-TAMRA | 23 | 705 | 332 |
| Reverse | 5′-ccaagaggttcacaaacactgt-3′ | 22 | 730 | 333 |

TABLE MB

Probe Name Ag3279

| | | | Start | SEQ ID |
|---|---|---|---|---|
| Primers | Sequences | Length | Position | No |
| Forward | 5′-gcaaaatcgtcgtctctgttac-3′ | 22 | 668 | 334 |
| Probe | TET-5′-ccctctgaaagccaccagcagtg-3′-TAMRA | 23 | 705 | 335 |
| Reverse | 5′-ccaagaggttcacaaacactgt-3′ | 22 | 730 | 336 |

TABLE MC

Probe Name Ag616

| | | | Start | SEQ ID |
|---|---|---|---|---|
| Primers | Sequences | Length | Position | No |
| Forward | 5′-tcgttgtccgtgcagttcag-3′ | 20 | 1156 | 337 |
| Probe | TET-5′-cagaccacccggaactcgcgtg-3′-TAMRA | 22 | 1133 | 338 |
| Reverse | 5′-cggccgtgtacaatgtgtct-3′ | 20 | 1097 | 339 |

TABLE MD
CNS_neurodegeneration_v1.0

| | Rel. Exp. (%) | Rel. Exp. (%) |
|---|---|---|
| | Ag3234, Run | Ag3279, Run |
| Tissue Name | 209862304 | 210060481 |
| AD 1 Hippo | 16.3 | 21.8 |
| AD 2 Hippo | 23.8 | 32.3 |
| AD 3 Hippo | 13.2 | 14.1 |
| AD 4 Hippo | 19.8 | 22.5 |
| AD 5 hippo | 58.2 | 49.7 |
| AD 6 Hippo | 58.6 | 100.0 |
| Control 2 Hippo | 19.8 | 24.5 |
| Control 4 Hippo | 29.1 | 23.5 |
| Control (Path) 3 Hippo | 11.7 | 7.1 |
| AD 1 Temporal Ctx | 28.1 | 28.7 |
| AD 2 Temporal Ctx | 18.7 | 28.9 |
| AD 3 Temporal Ctx | 15.5 | 15.7 |
| AD 4 Temporal Ctx | 27.7 | 25.5 |
| AD 5 Inf Temporal Ctx | 38.2 | 52.1 |
| AD 5 SupTemporal Ctx | 45.7 | 49.7 |
| AD 6 Inf Temporal Ctx | 81.8 | 67.8 |
| AD 6 Sup Temporal Ctx | 100.0 | 94.6 |
| Control 1 Temporal Ctx | 10.1 | 14.2 |
| Control 2 Temporal Ctx | 15.0 | 11.9 |
| Control 3 Temporal Ctx | 8.6 | 14.7 |
| Control 4 Temporal Ctx | 8.9 | 12.1 |
| Control (Path) 1 Temporal Ctx | 23.7 | 31.0 |
| Control (Path) 2 Temporal Ctx | 14.3 | 16.6 |
| Control (Path) 3 Temporal Ctx | 14.6 | 16.2 |
| Control (Path) 4 Temporal Ctx | 26.4 | 31.2 |
| AD 1 Occipital Ctx | 17.1 | 19.2 |
| AD 2 Occipital Ctx (Missing) | 0.0 | 0.0 |
| AD 3 Occipital Ctx | 23.5 | 22.4 |
| AD 4 Occipital Ctx | 18.3 | 21.2 |
| AD 5 Occipital Ctx | 31.9 | 38.4 |
| AD 6 Occipital Ctx | 24.1 | 37.9 |
| Control 1 Occipital Ctx | 19.8 | 26.1 |
| Control 2 Occipital Ctx | 31.0 | 33.7 |
| Control 3 Occipital Ctx | 21.8 | 22.7 |
| Control 4 Occipital Ctx | 13.3 | 20.7 |
| Control (Path) 1 Occipital Ctx | 37.1 | 32.5 |
| Control (Path) 2 Occipital Ctx | 14.7 | 12.6 |
| Control (Path) 3 Occipital Ctx | 20.3 | 19.2 |
| Control (Path) 4 Occipital Ctx | 20.4 | 26.1 |
| Control 1 Parietal Ctx | 14.2 | 22.4 |
| Control 2 Parietal Ctx | 35.8 | 44.4 |
| Control 3 Parietal Ctx | 5.5 | 12.8 |
| Control (Path) 1 Parietal Ctx | 17.7 | 21.9 |
| Control (Path) 2 Parietal Ctx | 19.2 | 25.3 |
| Control (Path) 3 Parietal Ctx | 13.0 | 17.9 |
| Control (Path) 4 Parietal Ctx | 32.3 | 33.0 |

TABLE ME
General_screening_panel_v1.4

| | Rel. Exp. (%) |
|---|---|
| | Ag3279, Run |
| Tissue Name | 216512994 |
| Adipose | 35.8 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.1 |
| Squamous cell carcinoma SCC-4 | 0.0 |
| Testis Pool | 11.0 |
| Prostate ca.* (bone met) PC-3 | 0.1 |
| Prostate Pool | 3.8 |
| Placenta | 0.6 |
| Uterus Pool | 3.1 |
| Ovarian ca. OVCAR-3 | 10.4 |
| Ovarian ca. SK-OV-3 | 1.4 |
| Ovarian ca. OVCAR-4 | 0.0 |
| Ovarian ca. OVCAR-5 | 4.0 |
| Ovarian ca. IGROV-1 | 2.4 |
| Ovarian ca. OVCAR-8 | 1.4 |
| Ovary | 37.4 |
| Breast ca. MCF-7 | 0.1 |
| Breast ca. MDA-MB-231 | 0.0 |
| Breast ca. BT 549 | 0.0 |
| Breast ca. T47D | 5.8 |
| Breast ca. MDA-N | 0.0 |
| Breast Pool | 7.0 |
| Trachea | 4.3 |
| Lung | 16.0 |
| Fetal Lung | 24.5 |
| Lung ca. NCI-N417 | 2.2 |
| Lung ca. LX-1 | 0.3 |
| Lung ca. NCI-H146 | 0.3 |
| Lung ca. SHP-77 | 0.0 |
| Lung ca. A549 | 4.2 |
| Lung ca. NCI-H526 | 10.2 |
| Lung ca. NCI-H23 | 12.0 |
| Lung ca. NCI-H460 | 0.1 |
| Lung ca. HOP-62 | 0.0 |
| Lung ca. NCI-H522 | 6.0 |
| Liver | 9.1 |
| Fetal Liver | 3.5 |
| Liver ca. HepG2 | 0.0 |
| Kidney Pool | 34.2 |
| Fetal Kidney | 5.0 |
| Renal ca. 786-0 | 0.0 |
| Renal ca. A498 | 1.1 |
| Renal ca. ACHN | 0.0 |
| Renal ca. UO-31 | 0.1 |
| Renal ca. TK-10 | 0.0 |
| Bladder | 9.4 |
| Gastric ca. (liver met.) NCI-N87 | 0.4 |
| Gastric ca. KATO III | 0.0 |
| Colon ca. SW-948 | 0.0 |
| Colon ca. SW480 | 2.1 |
| Colon ca.* (SW480 met) SW620 | 0.7 |
| Colon ca. HT29 | 0.0 |
| Colon ca. HCT-116 | 0.2 |
| Colon ca. CaCo-2 | 1.0 |
| Colon cancer tissue | 0.8 |
| Colon ca. SW1116 | 0.0 |
| Colon ca. Colo-205 | 0.0 |
| Colon ca. SW-48 | 0.0 |
| Colon Pool | 8.0 |
| Small Intestine Pool | 12.1 |
| Stomach Pool | 6.1 |
| Bone Marrow Pool | 8.4 |
| Fetal Heart | 15.6 |
| Heart Pool | 9.4 |
| Lymph Node Pool | 13.7 |
| Fetal Skeletal Muscle | 4.7 |
| Skeletal Muscle Pool | 4.9 |
| Spleen Pool | 5.8 |
| Thymus Pool | 13.0 |
| CNS cancer (glio/astro) U87-MG | 0.2 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.1 |
| CNS cancer (glio) SNB-19 | 1.8 |
| CNS cancer (glio) SF-295 | 0.8 |
| Brain (Amygdala) Pool | 9.7 |
| Brain (cerebellum) | 100.0 |
| Brain (fetal) | 8.2 |
| Brain (Hippocampus) Pool | 10.2 |
| Cerebral Cortex Pool | 7.9 |
| Brain (Substantia nigra) Pool | 6.0 |
| Brain (Thalamus) Pool | 10.9 |
| Brain (whole) | 16.0 |
| Spinal Cord Pool | 8.9 |
| Adrenal Gland | 3.9 |
| Pituitary gland Pool | 0.6 |
| Salivary Gland | 2.5 |
| Thyroid (female) | 1.4 |
| Pancreatic ca. CAPAN2 | 0.0 |
| Pancreas Pool | 9.5 |

TABLE MF
Panel 1.1

| | Rel. Exp. (%) |
|---|---|
| | Ag616, Run |
| Tissue Name | 111162134 |
| Adrenal gland | 3.7 |
| Bladder | 11.7 |
| Brain (amygdala) | 0.2 |
| Brain (cerebellum) | 76.8 |
| Brain (hippocampus) | 7.4 |
| Brain (substantia nigra) | 76.3 |
| Brain (thalamus) | 16.2 |
| Cerebral Cortex | 6.0 |
| Brain (fetal) | 2.2 |
| Brain (whole) | 31.0 |
| glio/astro U-118-MG | 0.0 |
| astrocytoma SF-539 | 0.0 |
| astrocytoma SNB-75 | 0.0 |
| astrocytoma SW1783 | 0.0 |
| glioma U251 | 0.0 |
| glioma SF-295 | 0.0 |
| glioma SNB-19 | 0.0 |
| glio/astro U87-MG | 0.0 |
| neuro*; met SK-N-AS | 0.0 |
| Mammary gland | 25.0 |
| Breast ca. BT-549 | 0.0 |
| Breast ca. MDA-N | 0.0 |
| Breast ca. * (pl. ef) T47D | 0.0 |
| Breast ca.* (pl. ef) MCF-7 | 0.0 |
| Breast ca.* (pl. ef) MDA-MB-231 | 0.0 |
| Small intestine | 2.4 |
| Colorectal | 0.0 |
| Colon ca. HT29 | 0.0 |
| Colon ca. CaCo-2 | 0.0 |
| Colon ca. HCT-15 | 0.0 |
| Colon ca. HCT-116 | 0.0 |
| Colon ca. HCC-2998 | 0.0 |
| Colon ca. SW480 | 0.0 |
| Colon ca.* SW620 (SW480 met) | 0.0 |
| Stomach | 3.4 |
| Gastric ca. (liver met) NCI-N87 | 0.0 |
| Heart | 46.3 |
| Skeletal muscle (Fetal) | 19.2 |
| Skeletal muscle | 19.2 |
| Endothelial cells | 0.0 |
| Heart (Fetal) | 4.1 |
| Kidney | 0.6 |
| Kidney (fetal) | 0.2 |
| Renal ca. 786-0 | 0.0 |
| Renal ca. A498 | 0.0 |
| Renal ca. ACHN | 0.0 |
| Renal ca. TK-10 | 0.0 |
| Renal ca. UO-31 | 0.0 |
| Renal ca. RXF 393 | 0.0 |
| Liver | 26.2 |
| Liver (fetal) | 0.1 |
| Liver ca. (hepatoblast) HepG2 | 0.0 |
| Lung | 4.2 |
| Lung (fetal) | 3.8 |
| Lung ca. (non-s. cell) HOP-62 | 0.0 |
| Lung ca. (large cell)NCI-H460 | 0.0 |
| Lung ca. (non-s. cell) NCI-H23 | 6.0 |
| Lung ca. (non-s. cl) NCI-H522 | 18.6 |
| Lung ca. (non-sm. cell) A549 | 6.2 |
| Lung ca. (s. cell var.) SHP-77 | 0.0 |
| Lung ca. (small cell) LX-1 | 0.0 |
| Lung ca. (small cell) NCI-H69 | 1.5 |
| Lung ca. (squam.) SW 900 | 0.0 |
| Lung ca. (squam.) NCI-H596 | 18.4 |
| Lymph node | 3.4 |
| Spleen | 2.9 |
| Thymus | 5.6 |
| Ovary | 47.3 |
| Ovarian ca. IGROV-1 | 8.6 |
| Ovarian ca. OVCAR-3 | 13.5 |
| Ovarian ca. OVCAR-4 | 0.0 |
| Ovarian ca. OVCAR-5 | 6.8 |
| Ovarian ca. OVCAR-8 | 0.2 |
| Ovarian ca.* (ascites) SK-OV-3 | 0.1 |
| Pancreas | 100.0 |
| Pancreatic ca. CAPAN 2 | 0.0 |
| Pituitary gland | 0.0 |
| Placenta | 0.5 |
| Prostate | 0.7 |
| Prostate ca.* (bone met) PC-3 | 0.0 |
| Salivary gland | 1.8 |
| Trachea | 1.0 |
| Spinal cord | 10.4 |
| Testis | 10.2 |
| Thyroid | 0.7 |
| Uterus | 7.6 |
| Melanoma M14 | 0.0 |
| Melanoma LOX IMVI | 0.0 |
| Melanoma UACC-62 | 0.0 |
| Melanoma SK-MEL-28 | 0.0 |
| Melanoma* (met) SK-MEL-5 | 0.0 |
| Melanoma Hs688(A).T | 0.0 |
| Melanoma* (met) Hs688(B).T | 0.0 |

TABLE MG
Panel 1.2

| | Rel. Exp. (%) |
|---|---|
| | Ag616, Run |
| Tissue Name | 118515000 |
| Endothelial cells | 0.0 |
| Heart (Fetal) | 8.5 |
| Pancreas | 100.0 |
| Pancreatic ca. CAPAN 2 | 0.0 |
| Adrenal Gland | 25.5 |
| Thyroid | 8.5 |
| Salivary gland | 8.1 |
| Pituitary gland | 6.2 |
| Brain (fetal) | 17.8 |
| Brain (whole) | 52.9 |
| Brain (amygdala) | 23.5 |
| Brain (cerebellum) | 66.4 |
| Brain (hippocampus) | 21.2 |
| Brain (thalamus) | 23.3 |
| Cerebral Cortex | 0.0 |
| Spinal cord | 19.3 |
| glio/astro U87-MG | 0.0 |
| glio/astro U-118-MG | 0.0 |
| astrocytoma SW1783 | 0.0 |
| neuro*; met SK-N-AS | 0.0 |
| astrocytoma SF-539 | 0.0 |
| astrocytoma SNB-75 | 0.0 |
| glioma SNB-19 | 0.1 |
| glioma U251 | 0.0 |
| glioma SF-295 | 0.0 |
| Heart | 69.7 |
| Skeletal Muscle | 32.1 |
| Bone marrow | 6.6 |
| Thymus | 17.4 |
| Spleen | 15.6 |
| Lymph node | 15.9 |
| Colorectal Tissue | 0.0 |
| Stomach | 11.8 |
| Small intestine | 15.9 |
| Colon ca. SW480 | 0.4 |
| Colon ca.* SW620 (SW480 met) | 0.2 |
| Colon ca. HT29 | 1.0 |
| Colon ca. HCT-116 | 0.0 |
| Colon ca. CaCo-2 | 0.7 |
| Colon ca. Tissue (ODO3866) | 0.3 |
| Colon ca. HCC-2998 | 0.6 |
| Gastric ca.* (liver met) NCI-N87 | 0.2 |
| Bladder | 28.3 |
| Trachea | 7.0 |
| Kidney | 2.8 |
| Kidney (fetal) | 5.3 |
| Renal ca. 786-0 | 0.0 |
| Renal ca. A498 | 0.1 |
| Renal ca. RXF 393 | 0.0 |
| Renal ca. ACHN | 0.0 |
| Renal ca. UO-31 | 0.0 |
| Renal ca. TK-10 | 0.0 |
| Liver | 69.7 |
| Liver (fetal) | 6.6 |
| Liver ca. (hepatoblast) HepG2 | 0.0 |
| Lung | 21.5 |
| Lung (fetal) | 12.0 |
| Lung ca. (small cell) LX-1 | 0.8 |
| Lung ca. (small cell) NCI-H69 | 8.4 |
| Lung ca. (s. cell var.) SHP-77 | 0.0 |
| Lung ca. (large cell)NCI-H460 | 0.0 |
| Lung ca. (non-sm. cell) A549 | 12.8 |
| Lung ca. (non-s. cell) NCI-H23 | 11.2 |
| Lung ca. (non-s. cell) HOP-62 | 0.1 |
| Lung ca. (non-s. cl) NCI-H522 | 36.3 |
| Lung ca. (squam.) SW 900 | 1.6 |
| Lung ca. (squam.) NCI-H596 | 29.5 |
| Mammary gland | 44.1 |
| Breast ca.* (pl. ef) MCF-7 | 0.0 |
| Breast ca.* (pl. ef) MDA-MB-231 | 0.0 |
| Breast ca.* (pl. ef) T47D | 0.1 |
| Breast ca. BT-549 | 0.0 |
| Breast ca. MDA-N | 0.0 |
| Ovary | 53.2 |
| Ovarian ca. OVCAR-3 | 18.9 |
| Ovarian ca. OVCAR-4 | 0.4 |
| Ovarian ca. OVCAR-5 | 10.5 |
| Ovarian ca. OVCAR-8 | 3.6 |
| Ovarian ca. IGROV-1 | 18.8 |
| Ovarian ca. (ascites) SK-OV-3 | 1.9 |
| Uterus | 22.7 |
| Placenta | 6.7 |
| Prostate | 6.5 |
| Prostate ca.* (bone met) PC-3 | 0.0 |
| Testis | 50.3 |
| Melanoma Hs688(A).T | 0.0 |
| Melanoma* (met) Hs688(B).T | 0.0 |
| Melanoma UACC-62 | 0.0 |
| Melanoma M14 | 0.0 |
| Melanoma LOX IMVI | 0.0 |
| Melanoma* (met) SK-MEL-5 | 0.0 |

TABLE MH
Panel 1.3D

| | Rel. Exp. (%) |
|---|---|
| | Ag3234, Run |
| Tissue Name | 165524160 |
| Liver adenocarcinoma | 5.8 |
| Pancreas | 43.2 |
| Pancreatic ca. CAPAN 2 | 0.0 |
| Adrenal gland | 4.5 |
| Thyroid | 0.0 |
| Salivary gland | 6.3 |
| Pituitary gland | 1.4 |
| Brain (fetal) | 5.8 |
| Brain (whole) | 45.4 |
| Brain (amygdala) | 27.2 |
| Brain (cerebellum) | 100.0 |
| Brain (hippocampus) | 21.3 |
| Brain (substantia nigra) | 21.0 |
| Brain (thalamus) | 27.2 |
| Cerebral Cortex | 14.3 |
| Spinal cord | 35.4 |
| glio/astro U87-MG | 0.0 |
| glio/astro U-118-MG | 0.0 |
| astrocytoma SW1783 | 0.0 |
| neuro*; met SK-N-AS | 1.6 |
| astrocytoma SF-539 | 0.0 |
| astrocytoma SNB-75 | 0.0 |
| glioma SNB-19 | 0.0 |
| glioma U251 | 0.0 |
| glioma SF-295 | 0.0 |
| Heart (fetal) | 15.2 |
| Heart | 13.3 |
| Skeletal muscle (fetal) | 9.9 |
| Skeletal muscle | 11.2 |
| Bone marrow | 6.2 |
| Thymus | 18.6 |
| Spleen | 8.0 |
| Lymph node | 14.4 |
| Colorectal | 7.4 |
| Stomach | 4.5 |
| Small intestine | 16.6 |
| Colon ca. SW480 | 0.0 |
| Colon ca.* SW620 (SW480 met) | 0.0 |
| Colon ca. HT29 | 0.0 |
| Colon ca. HCT-116 | 0.0 |
| Colon ca. CaCo-2 | 0.0 |
| Colon ca. tissue (ODO3866) | 1.4 |
| Colon ca. HCC-2998 | 0.0 |
| Gastric ca.* (liver met) NCI-N87 | 0.0 |
| Bladder | 5.2 |
| Trachea | 4.1 |
| Kidney | 0.0 |
| Kidney (fetal) | 2.4 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.0 |
| Renal ca. TK-10 | 0.0 |
| Liver | 6.3 |
| Liver (fetal) | 2.9 |
| Liver ca. (hepatoblast) HepG2 | 0.0 |
| Lung | 12.2 |
| Lung (fetal) | 6.5 |
| Lung ca. (small cell) LX-1 | 1.4 |
| Lung ca. (small cell) NCI-H69 | 1.2 |
| Lung ca. (s. cell var.) SHP-77 | 0.0 |
| Lung ca. (large cell)NCI-H460 | 2.0 |
| Lung ca. (non-sm. cell) A549 | 4.9 |
| Lung ca. (non-s. cell) NCI-H23 | 6.2 |
| Lung ca. (non-s. cell) HOP-62 | 0.0 |
| Lung ca. (non-s. cl) NCI-H522 | 0.6 |
| Lung ca. (squam.) SW 900 | 0.7 |
| Lung ca. (squam.) NCI-H596 | 7.4 |
| Mammary gland | 28.9 |
| Breast ca.* (pl. ef) MCF-7 | 0.0 |
| Breast ca.* (pl. ef) MDA-MB-231 | 0.0 |
| Breast ca.* (pl. ef) T47D | 0.0 |
| Breast ca. BT-549 | 0.0 |
| Breast ca. MDA-N | 0.0 |
| Ovary | 50.7 |
| Ovarian ca. OVCAR-3 | 7.1 |
| Ovarian ca. OVCAR-4 | 1.3 |
| Ovarian ca. OVCAR-5 | 6.0 |
| Ovarian ca. OVCAR-8 | 3.0 |
| Ovarian ca. IGROV-1 | 1.6 |
| Ovarian ca.* (ascites) SK-OV-3 | 1.2 |
| Uterus | 42.6 |
| Placenta | 1.7 |
| Prostate | 3.4 |
| Prostate ca.* (bone met) PC-3 | 0.0 |
| Testis | 15.6 |
| Melanoma Hs688(A).T | 0.0 |
| Melanoma* (met) Hs688(B).T | 0.0 |
| Melanoma UACC-62 | 0.0 |
| Melanoma M14 | 0.0 |
| Melanoma LOX IMVI | 0.0 |
| Melanoma* (met) SK-MEL-5 | 0.0 |
| Adipose | 28.1 |

TABLE MI
Panel 2.2

| | Rel. Exp. (%) |
|---|---|
| | Ag3234, Run |
| Tissue Name | 174442923 |
| Normal Colon | 7.1 |
| Colon cancer (OD06064) | 6.7 |
| Colon Margin (OD06064) | 5.6 |
| Colon cancer (OD06159) | 0.0 |
| Colon Margin (OD06159) | 5.9 |
| Colon cancer (OD06297-04) | 0.0 |
| Colon Margin (OD06297-05) | 2.9 |
| CC Gr 2 ascend colon (ODO3921) | 1.8 |
| CC Margin (ODO3921) | 0.0 |
| Colon cancer metastasis (OD06104) | 1.2 |
| Lung Margin (OD06104) | 0.7 |
| Colon mets to lung (OD04451-01) | 0.0 |
| Lung Margin (OD04451-02) | 57.4 |
| Normal Prostate | 2.6 |
| Prostate Cancer (OD04410) | 0.0 |
| Prostate Margin (OD04410) | 7.7 |
| Normal Ovary | 100.0 |
| Ovarian cancer (OD06283-03) | 5.4 |
| Ovarian Margin (OD06283-07) | 27.9 |
| Ovarian Cancer 064008 | 12.9 |
| Ovarian cancer (OD06145) | 12.7 |
| Ovarian Margin (OD06145) | 19.3 |
| Ovarian cancer (OD06455-03) | 8.1 |
| Ovarian Margin (OD06455-07) | 25.9 |
| Normal Lung | 14.8 |
| Invasive poor diff. lung adeno (ODO4945-01 | 0.5 |
| Lung Margin (ODO4945-03) | 28.7 |
| Lung Malignant Cancer (OD03126) | 7.0 |
| Lung Margin (OD03126) | 3.2 |
| Lung Cancer (OD05014A) | 3.8 |
| Lung Margin (OD05014B) | 28.5 |
| Lung cancer (OD06081) | 3.1 |
| Lung Margin (OD06081) | 18.2 |
| Lung Cancer (OD04237-01) | 2.7 |
| Lung Margin (OD04237-02) | 12.2 |
| Ocular Melanoma Metastasis | 0.0 |
| Ocular Melanoma Margin (Liver) | 22.2 |
| Melanoma Metastasis | 0.0 |
| Melanoma Margin (Lung) | 18.4 |
| Normal Kidney | 1.2 |
| Kidney Ca, Nuclear grade 2 (OD04338) | 6.7 |
| Kidney Margin (OD04338) | 1.7 |
| Kidney Ca Nuclear grade 1/2 (OD04339) | 22.1 |
| Kidney Margin (OD04339) | 5.0 |
| Kidney Ca, Clear cell type (OD04340) | 3.5 |
| Kidney Margin (OD04340) | 3.7 |
| Kidney Ca, Nuclear grade 3 (OD04348) | 0.8 |
| Kidney Margin (OD04348) | 4.8 |
| Kidney malignant cancer (OD06204B) | 22.2 |
| Kidney normal adjacent tissue (OD06204E) | 3.4 |
| Kidney Cancer (OD04450-01) | 0.0 |
| Kidney Margin (OD04450-03) | 1.8 |
| Kidney Cancer 8120613 | 0.0 |
| Kidney Margin 8120614 | 1.4 |
| Kidney Cancer 9010320 | 0.0 |
| Kidney Margin 9010321 | 1.3 |
| Kidney Cancer 8120607 | 1.9 |
| Kidney Margin 8120608 | 1.5 |
| Normal Uterus | 33.9 |
| Uterine Cancer 064011 | 6.7 |
| Normal Thyroid | 1.3 |
| Thyroid Cancer 064010 | 0.0 |
| Thyroid Cancer A302152 | 1.8 |
| Thyroid Margin A302153 | 0.8 |
| Normal Breast | 22.5 |
| Breast Cancer (OD04566) | 3.9 |
| Breast Cancer 1024 | 11.4 |
| Breast Cancer (OD04590-01) | 20.0 |
| Breast Cancer Mets (OD04590-03) | 17.2 |
| Breast Cancer Metastasis (OD04655-05) | 31.0 |
| Breast Cancer 064006 | 3.9 |
| Breast Cancer 9100266 | 10.0 |
| Breast Margin 9100265 | 16.8 |
| Breast Cancer A209073 | 10.4 |
| Breast Margin A2090734 | 28.1 |
| Breast cancer (OD06083) | 12.6 |
| Breast cancer node metastasis (OD06083) | 13.7 |
| Normal Liver | 41.5 |
| Liver Cancer 1026 | 10.5 |
| Liver Cancer 1025 | 40.1 |
| Liver Cancer 6004-T | 21.0 |
| Liver Tissue 6004-N | 3.4 |
| Liver Cancer 6005-T | 59.9 |
| Liver Tissue 6005-N | 59.5 |
| Liver Cancer 064003 | 10.4 |
| Normal Bladder | 7.6 |
| Bladder Cancer 1023 | 2.5 |
| Bladder Cancer A302173 | 1.4 |
| Normal Stomach | 12.3 |
| Gastric Cancer 9060397 | 1.0 |
| Stomach Margin 9060396 | 1.6 |
| Gastric Cancer 9060395 | 1.3 |
| Stomach Margin 9060394 | 3.1 |
| Gastric Cancer 064005 | 0.0 |

TABLE MJ
Panel 4D

| | Rel. | Rel. |
|---|---|---|
| | Exp. (%) | Exp. (%) |
| | Ag3234, | Ag3279, |
| | Run | Run |
| Tissue Name | 164328482 | 164634320 |
| Secondary Th1 act | 0.0 | 0.0 |
| Secondary Th2 act | 0.2 | 0.0 |
| Secondary Tr1 act | 0.0 | 0.0 |
| Secondary Th1 rest | 0.6 | 0.2 |
| Secondary Th2 rest | 0.0 | 0.0 |
| Secondary Tr1 rest | 0.3 | 0.1 |
| Primary Th1 act | 0.2 | 0.1 |
| Primary Th2 act | 0.0 | 0.0 |
| Primary Tr1 act | 0.0 | 0.9 |
| Primary Th1 rest | 0.7 | 0.9 |
| Primary Th2 rest | 0.2 | 0.2 |
| Primary Tr1 rest | 0.8 | 0.2 |
| CD45RA CD4 lymphocyte act | 0.0 | 0.0 |
| CD45RO CD4 lymphocyte act | 0.0 | 0.0 |
| CD8 lymphocyte act | 0.0 | 0.0 |
| Secondary CD8 lymphocyte rest | 0.0 | 0.0 |
| Secondary CD8 lymphocyte act | 0.0 | 0.0 |
| CD4 lymphocyte none | 0.2 | 0.7 |
| 2ry Th1/Th2/Tr1_anti-CD95 CH11 | 0.2 | 0.0 |
| LAK cells rest | 6.2 | 7.6 |
| LAK cells IL-2 | 0.0 | 0.0 |
| LAK cells IL-2 + IL-12 | 0.0 | 0.0 |
| LAK cells IL-2 + IFN gamma | 0.1 | 0.4 |
| LAK cells IL-2 + IL-18 | 0.4 | 0.0 |
| LAK cells PMA/ionomycin | 1.3 | 2.7 |
| NK Cells IL-2 rest | 0.0 | 0.0 |
| Two Way MLR 3 day | 1.1 | 1.3 |
| Two Way MLR 5 day | 1.3 | 0.6 |
| Two Way MLR 7 day | 0.8 | 0.4 |
| PBMC rest | 3.3 | 3.1 |
| PBMC PWM | 0.0 | 0.1 |
| PBMC PHA-L | 0.1 | 0.0 |
| Ramos (B cell) none | 0.0 | 0.0 |
| Ramos (B cell) ionomycin | 0.0 | 0.0 |
| B lymphocytes PWM | 0.0 | 0.0 |
| B lymphocytes CD40L and IL-4 | 0.0 | 0.3 |
| EOL-1 dbcAMP | 0.0 | 0.2 |
| EOL-1 dbcAMP PMA/ionomycin | 0.1 | 0.0 |
| Dendritic cells none | 25.9 | 49.0 |
| Dendritic cells LPS | 61.1 | 92.0 |
| Dendritic cells anti-CD40 | 100.0 | 94.6 |
| Monocytes rest | 12.2 | 23.0 |
| Monocytes LPS | 2.6 | 2.5 |
| Macrophages rest | 92.0 | 100.0 |
| Macrophages LPS | 10.4 | 18.0 |
| HUVEC none | 0.0 | 0.0 |
| HUVEC starved | 0.0 | 0.0 |
| HUVEC IL-1beta | 0.0 | 0.0 |
| HUVEC IFN gamma | 0.0 | 0.0 |
| HUVEC TNF alpha + IFN gamma | 0.0 | 0.0 |
| HUVEC TNF alpha + IL4 | 0.0 | 0.0 |
| HUVEC IL-11 | 0.0 | 0.0 |
| Lung Microvascular EC none | 0.0 | 0.0 |
| Lung Microvascular EC | 0.0 | 0.0 |
| TNFalpha + IL-1beta | | |
| Microvascular Dermal EC none | 0.0 | 0.0 |
| Microsvasular Dermal EC | 0.0 | 0.0 |
| TNFalpha + IL-1beta | | |
| Bronchial epithelium | 0.0 | 0.0 |
| TNFalpha + IL1beta | | |
| Small airway epithelium none | 0.0 | 0.0 |
| Small airway epithelium | 0.0 | 0.0 |
| TNFalpha + IL-1beta | | |
| Coronery artery SMC rest | 0.0 | 0.0 |
| Coronery artery SMC | 0.0 | 0.0 |
| TNFalpha + IL-1beta | | |
| Astrocytes rest | 0.0 | 0.0 |
| Astrocytes | 0.0 | 0.0 |
| TNFalpha + IL-1beta | | |
| KU-812 (Basophil) rest | 0.5 | 0.0 |
| KU-812 (Basophil) PMA/ionomycin | 0.2 | 0.3 |
| CCD1106 (Keratinocytes) none | 0.0 | 0.0 |
| CCD1106 (Keratinocytes) | 0.0 | 0.0 |
| TNFalpha + IL-1beta | | |
| Liver cirrhosis | 1.4 | 1.2 |
| Lupus kidney | 0.4 | 0.2 |
| NCI-H292 none | 0.0 | 0.2 |
| NCI-H292 IL-4 | 0.0 | 0.0 |
| NCI-H292 IL-9 | 0.0 | 0.0 |
| NCI-H292 IL-13 | 0.0 | 0.0 |
| NCI-H292 IFN gamma | 0.0 | 0.2 |
| HPAEC none | 0.0 | 0.0 |
| HPAEC TNF alpha + IL-1 beta | 0.0 | 0.0 |
| Lung fibroblast none | 0.0 | 0.0 |
| Lung fibroblast | 0.0 | 0.0 |
| TNF alpha + IL-1 beta | | |
| Lung fibroblast IL-4 | 0.0 | 0.0 |
| Lung fibroblast IL-9 | 0.0 | 0.0 |
| Lung fibroblast IL-13 | 0.0 | 0.0 |
| Lung fibroblast IFN gamma | 0.0 | 0.0 |
| Dermal fibroblast CCD1070 rest | 0.0 | 0.0 |
| Dermal fibroblast CCD1070 TNF | 0.2 | 0.6 |
| alpha | | |
| Dermal fibroblast | 0.0 | 0.0 |
| CCD1070 IL-1beta | | |
| Dermal fibroblast IFN gamma | 0.0 | 0.2 |
| Dermal fibroblast IL-4 | 0.2 | 0.0 |
| IBD Colitis 2 | 0.0 | 0.7 |
| IBD Crohn's | 0.3 | 0.7 |
| Colon | 3.0 | 2.8 |
| Lung | 7.4 | 9.7 |
| Thymus | 1.2 | 3.7 |
| Kidney | 35.4 | 33.4 |

TABLE MK
Panel CNS_1

| | Rel. Exp. (%) |
|---|---|
| | Ag3279, Run |
| Tissue Name | 171694591 |
| BA4 Control | 3.2 |
| BA4 Control2 | 10.0 |
| BA4 Alzheimer's2 | 3.8 |
| BA4 Parkinson's | 6.5 |
| BA4 Parkinson's2 | 11.7 |
| BA4 Huntington's | 7.6 |
| BA4 Huntington's2 | 4.5 |
| BA4 PSP | 5.1 |
| BA4 PSP2 | 8.8 |
| BA4 Depression | 6.7 |
| BA4 Depression2 | 7.4 |
| BA7 Control | 4.5 |
| BA7 Control2 | 1.9 |
| BA7 Alzheimer's2 | 1.0 |
| BA7 Parkinson's | 24.1 |
| BA7 Parkinson's2 | 17.4 |
| BA7 Huntington's | 6.3 |
| BA7 Huntington's2 | 28.5 |
| BA7 PSP | 18.9 |
| BA7 PSP2 | 3.8 |
| BA7 Depression | 1.7 |
| BA9 Control | 5.3 |
| BA9 Control2 | 4.1 |
| BA9 Alzheimer's | 3.6 |
| BA9 Alzheimer's2 | 8.8 |
| BA9 Parkinson's | 18.6 |
| BA9 Parkinson's2 | 20.4 |
| BA9 Huntington's | 15.0 |
| BA9 Huntington's2 | 7.4 |
| BA9 PSP | 5.3 |
| BA9 PSP2 | 1.6 |
| BA9 Depression | 4.7 |
| BA9 Depression2 | 4.5 |
| BA17 Control | 20.2 |
| BA17 Control2 | 7.9 |
| BA17 Alzheimer's2 | 3.2 |
| BA17 Parkinson's | 14.1 |
| BA17 Parkinson's2 | 8.7 |
| BA17 Huntington's | 22.2 |
| BA17 Huntington's2 | 18.2 |
| BA17 Depression | 4.9 |
| BA17 Depression2 | 19.9 |
| BA17 PSP | 13.4 |
| BA17 PSP2 | 5.3 |
| Sub Nigra Control | 58.6 |
| Sub Nigra Control2 | 21.3 |
| Sub Nigra Alzheimer's2 | 12.2 |
| Sub Nigra Parkinson's2 | 63.3 |
| Sub Nigra Huntington's | 100.0 |
| Sub Nigra Huntington's2 | 87.1 |
| Sub Nigra PSP2 | 16.8 |
| Sub Nigra Depression | 7.9 |
| Sub Nigra Depression2 | 24.3 |
| Glob Palladus Control | 24.3 |
| Glob Palladus Control2 | 8.1 |
| Glob Palladus Alzheimer's | 14.9 |
| Glob Palladus Alzheimer's2 | 1.2 |
| Glob Palladus Parkinson's | 33.0 |
| Glob Palladus Parkinson's2 | 2.0 |
| Glob Palladus PSP | 3.0 |
| Glob Palladus PSP2 | 6.0 |
| Glob Palladus Depression | 8.8 |
| Temp Pole Control | 5.9 |
| Temp Pole Control2 | 11.7 |
| Temp Pole Alzheimer's | 7.4 |
| Temp Pole Alzheimer's2 | 3.0 |
| Temp Pole Parkinson's | 11.9 |
| Temp Pole Parkinson's2 | 7.9 |
| Temp Pole Huntington's | 8.8 |
| Temp Pole PSP | 4.7 |
| Temp Pole PSP2 | 0.0 |
| Temp Pole Depression2 | 12.6 |
| Cing Gyr Control | 18.0 |
| Cing Gyr Control2 | 20.2 |
| Cing Gyr Alzheimer's | 8.6 |
| Cing Gyr Alzheimer's2 | 4.2 |
| Cing Gyr Parkinson's | 12.2 |
| Cing Gyr Parkinson's2 | 15.3 |
| Cing Gyr Huntington's | 28.1 |
| Cing Gyr Huntington's2 | 4.7 |
| Cing Gyr PSP | 7.8 |
| Cing Gyr PSP2 | 11.2 |
| Cing Gyr Depression | 2.9 |
| Cing Gyr Depression2 | 8.7 |

TABLE ML
general oncology screening panel_v_2.4

| | Rel. | Rel. |
|---|---|---|
| | Exp. (%) | Exp. (%) |
| | Ag3279, Run | Ag3279, Run |
| Tissue Name | 264978500 | 267936331 |
| Colon cancer 1 | 0.7 | 0.6 |
| Colon cancer NAT 1 | 1.3 | 1.5 |
| Colon cancer 2 | 3.3 | 4.1 |
| Colon cancer NAT 2 | 0.7 | 0.6 |
| Colon cancer 3 | 1.6 | 1.4 |
| Colon cancer NAT 3 | 2.2 | 2.1 |
| Colon malignant cancer 4 | 1.7 | 1.9 |
| Colon normal adjacent tissue 4 | 1.3 | 0.9 |
| Lung cancer 1 | 3.8 | 3.6 |
| Lung NAT 1 | 1.5 | 1.7 |
| Lung cancer 2 | 4.5 | 7.2 |
| Lung NAT 2 | 4.1 | 6.3 |
| Squamous cell carcinoma 3 | 4.7 | 7.0 |
| Lung NAT 3 | 0.2 | 0.4 |
| metastatic melanoma 1 | 46.0 | 40.3 |
| Melanoma 2 | 1.4 | 0.8 |
| Melanoma 3 | 2.3 | 1.7 |
| metastatic melanoma 4 | 67.4 | 80.1 |
| metastatic melanoma 5 | 100.0 | 100.0 |
| Bladder cancer 1 | 0.6 | 1.0 |
| Bladder cancer NAT 1 | 0.0 | 0.0 |
| Bladder cancer 2 | 1.9 | 0.9 |
| Bladder cancer NAT 2 | 0.4 | 0.2 |
| Bladder cancer NAT 3 | 0.3 | 0.7 |
| Bladder cancer NAT 4 | 2.5 | 0.8 |
| Prostate adenocarcinoma 1 | 54.0 | 70.7 |
| Prostate adenocarcinoma 2 | 2.2 | 2.4 |
| Prostate adenocarcinoma 3 | 4.5 | 6.2 |
| Prostate adenocarcinoma 4 | 2.1 | 0.8 |
| Prostate cancer NAT 5 | 1.9 | 4.8 |
| Prostate adenocarcinoma 6 | 1.0 | 1.5 |
| Prostate adenocarcinoma 7 | 4.0 | 3.3 |
| Prostate adenocarcinoma 8 | 0.9 | 1.6 |
| Prostate adenocarcinoma 9 | 60.7 | 54.3 |
| Prostate cancer NAT 10 | 0.5 | 0.8 |
| Kidney cancer 1 | 4.1 | 6.0 |
| KidneyNAT 1 | 5.1 | 5.4 |
| Kidney cancer 2 | 8.1 | 7.5 |
| Kidney NAT 2 | 2.2 | 3.7 |
| Kidney cancer 3 | 2.3 | 1.4 |
| Kidney NAT 3 | 1.9 | 1.4 |
| Kidney cancer 4 | 1.8 | 1.8 |
| Kidney NAT 4 | 0.6 | 1.9 |

CNS_neurodegeneration_v1.0 Summary:

Ag3234/Ag3279 Two experiments with the same probe and primer set produce results that are in excellent agreement. Both experiments show a difference in expression of this gene between Alzheimer's diseased postmortem brains and controls for this gene. Expression is increased in the temporal cortex of patients with AD (p=0.016 for ag3234 and p=0.024 for ag3279) and in the hippocampus. Both the temporal cortex and hippocampus are regions that show severe neurodegeneration in AD. In contrast, expression in the occipital cortex, a region that does not degenerate in Alzheimer's disease, is not disregulated. Together, these data suggest that the Cadherin protein encoded by this gene may be involved in the pathology or response to Alzheimer's disease. Therefore, this may be a useful drug target for the treatment of this disease.

General_Screening_Panel_v1.4 Summary:

Ag3279 Highest expression of this gene is in the cerebellum (CT=25.9). Significant levels of expression are also seen in other regions of the brain including the amygdala, hippocampus, cerebral cortex, substantia nigra, and thalamus. Cadherins can act as axon guidance and cell adhesion proteins, specifically during development and in the response to injury (ref 1). Manipulation of levels of this protein may be of use in inducing a compensatory synaptogenic response to neuronal death in Alzheimer's disease, Parkinson's disease, Huntington's disease, spinocerebellar ataxia, progressive supranuclear palsy, ALS, head trauma, stroke, or any other disease/condition associated with neuronal loss.

In addition, this gene is highly expressed in pituitary gland, adrenal gland, thyroid, pancreas, adult and fetal skeletal muscle, heart and liver, reflecting the widespread role of cadherins in cell-cell adhesion. This observation may suggest that this gene plays a role in normal metabolic and neuroendocrine function and that disregulated expression of this gene may contribute to metabolic diseases (such as obesity and diabetes) or neuroendocrine disorders.

Overall, gene expression is associated with normal tissues rather than cancer cell lines. Loss of function of the related E-cadherin protein has been described in many tumors, along with an increased invasiveness and a decreased prognosis of many carcinomas, including tumors of endocrine glands and their target systems (ref 2). Thus, this gene product might similarly be useful as a protein therapeutic to treat a variety of tumors, since it is found in normal cells but missing from cancer cells.

References:

1. Ranscht B. (2000) Cadherins: molecular codes for axon guidance and synapse formation. Int. J. Dev. Neurosci. 18: 643-651. PMID: 10978842

2. Potter E., Bergwitz C., Brabant G. (1999) The cadherin-catenin system: implications for growth and differentiation of endocrine tissues. Endocr. Rev. 20: 207-239. PMID: 10204118

Panel 1.1 Summary:

Ag616 Highest expression of this gene, a cadherin homolog, is seen in pancreas (CT=23.2). Significant expression is also seen in adrenal gland, fetal and adult skeletal muscle, liver and heart. This widespread expression among tissues with metabolic function is consistent with expression seen in General_screening_panel_v1.4. Please see that panel for further discussion of utility of this gene in metabolic disorders.

In addition, there is higher expression in adult liver (CT=27) when compared to expression in fetal liver (CT=34.8). Thus, expression of this gene could be used to differentiate between fetal and adult liver.

Overall, expression in this panel is in agreement with expression in the previous panel. Please see that panel for further discussion of utility of this gene.

Panel 1.2 Summary:

Ag616 The expression of this gene in this panel is in agreement with expression in the panels 1.1 and 1.4. Please see these panels for further discussion of utility of this gene.

Panel 1.3D Summary:

Ag3234 The expression of this gene in this panel is in agreement with expression in the panels 1.4. See panel 1.4 for further discussion.

Panel 2.2 Summary:

Ag3234 The expression of this gene appears to be highest in a sample derived from a normal ovarian tissue (CT=32.3). In addition, there appears to be substantial expression in other samples derived from liver cancers. Furthermore, there appears to be expression specific to normal lung tissue when compared to malignant lung tissue. Thus, the expression of this gene could be used to distinguish normal ovarian tissue from other samples in the panel. Moreover, therapeutic modulation of this gene, through the use of small molecule drugs, protein therapeutics or antibodies could be of benefit in the treatment of liver cancer, ovarian cancer or lung cancer.

Panel 4D Summary:

Ag3234/Ag3279 The this gene, a cadherin 23-like molecule, is expressed selectively at moderate levels (CTs=28.1-30.1) in resting and activated dendritic cells, and in resting and activated macrophages. Thus, small molecule antagonists or therapeutic antibodies that block the function of the cadherin 23-like molecule encoded by this gene may be useful in the reduction or elimination of the symptoms in patients with autoimmune and inflammatory diseases in which dendritic cells and macrophages play an important role in antigen presentation and other functions, such as, but not limited to, including Crohn's disease, ulcerative colitis, multiple sclerosis, chronic obstructive pulmonary disease, asthma, emphysema, rheumatoid arthritis, lupus erythematosus, or psoriasis.

Panel CNS_{—}1 Summary:

Ag3279 This panel confirms expression of this gene in the brain. See Panel 1.4 for discussion of utility of this gene in the central nervous system.

General Oncology Screening Panel_V_{—}2.4 Summary:

Ag3234/Ag3279 Two experiments with same probe-primer sets are in excellent agreement. Highest expression of this gene is detected in metastatic melanoma sample (CTs=27-29). High expression of this gene is detected in metastic melanoma and prostate adenocarcinoma. Therefore, expression of this gene may be used as diagnostic marker to detect the presence of prostate cancer and metastatic melanoma. In addition, moderate to low levels of expression of this gene is also detected in normal and cancer samples derived from colon, lung and kidney. Therefore, therapeutic modulation of this gene or its protein product through the use of protein therapeutics, antibodies or small molecules may be useful in the treatment of metastatic melanoma, prostate, colon, lung and kidney cancers.

N. CG89285-03: Alpha-1-Antichymotrypsin.

Expression of gene CG89285-03 was assessed using the primer-probe set Ag5223, described in Table NA. Results of the RTQ-PCR runs are shown in Table NB.

TABLE NA

Probe Name Ag5223

| | | | Start | SEQ ID |
|---|---|---|---|---|
| Primers | Sequences | Length | Position | No |
| Forward | 5′-atggtcctggtgaattacat-3′ | 20 | 661 | 340 |
| Probe | TET-5′-cttctttaaagagagataggtgagctctac-3′-TAMRA | 30 | 681 | 341 |
| Reverse | 5′-ctcaaatacatcaagcacag-3′ | 20 | 856 | 342 |

TABLE NB
General_screening_panel_v1.5

| | Rel. Exp. (%) |
|---|---|
| | Ag5223, Run |
| Tissue Name | 229514473 |
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 13.9 |
| Squamous cell carcinoma SCC-4 | 0.0 |
| Testis Pool | 0.0 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 15.6 |
| Ovary | 0.0 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 9.1 |
| Lung | 0.0 |
| Fetal Lung | 4.8 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 0.0 |
| Lung ca. NCI-H526 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 11.0 |
| Fetal Liver | 32.8 |
| Liver ca. HepG2 | 100.0 |
| Kidney Pool | 0.0 |
| Fetal Kidney | 0.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 47.6 |
| Bladder | 80.7 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.0 |
| Stomach Pool | 4.0 |
| Bone Marrow Pool | 0.0 |
| Fetal Heart | 0.0 |
| Heart Pool | 0.0 |
| Lymph Node Pool | 0.0 |
| Fetal Skeletal Muscle | 0.0 |
| Skeletal Muscle Pool | 9.2 |
| Spleen Pool | 0.0 |
| Thymus Pool | 0.0 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 4.9 |
| CNS cancer (glio) SNB-19 | 0.0 |
| CNS cancer (glio) SF-295 | 21.2 |
| Brain (Amygdala) Pool | 0.0 |
| Brain (cerebellum) | 0.0 |
| Brain (fetal) | 0.0 |
| Brain (Hippocampus) Pool | 2.3 |
| Cerebral Cortex Pool | 0.0 |
| Brain (Substantia nigra) Pool | 0.0 |
| Brain (Thalamus) Pool | 0.0 |
| Brain (whole) | 3.2 |
| Spinal Cord Pool | 18.9 |
| Adrenal Gland | 0.0 |
| Pituitary gland Pool | 0.0 |
| Salivary Gland | 0.0 |
| Thyroid (female) | 0.0 |
| Pancreatic ca. CAPAN2 | 0.0 |
| Pancreas Pool | 9.1 |

CNS_Neurodegeneration_v1.0 Summary:

Ag5223 Expression of this gene is low/undetectable in all samples on this panel (CTs>35).

General_Screening_Panel_v1.5 Summary:

Ag5223 Expression of this gene is restricted to a sample derived from a liver cancer cell line (CT=34.5) and normal bladder. Thus, expression of this gene could be used to differentiate between this sample and other samples on this panel and as a marker to detect the presence of liver cancer. Furthermore, therapeutic modulation of the expression or function of this gene may be effective in the treatment of liver cancer.

Panel 4.1D Summary:

Ag5223 Expression of this gene is low/undetectable in all samples on this panel (CTs>35).

O. CG89285-04: Alpha-1-Antichymotrypsin.

Expression of gene CG89285-04 was assessed using the primer-probe set Ag523 1, described in Table OA. Results of the RTQ-PCR runs are shown in Table OB.

TABLE QA

Probe Name Ag5231

| | | | Start | SEQ ID |
|---|---|---|---|---|
| Primers | Sequences | Length | Position | No |
| Forward | 5′-ctgacctgtcaaggaccattg-3′ | 21 | 1085 | 343 |
| Probe | TET-5′-tcaacaggcccttcctgatgatcatt-3′-TAMRA | 26 | 1112 | 344 |
| Reverse | 5′-ccagtttgaattccaagttcct-3′ | 22 | 1232 | 345 |

TABLE OB
General_screening_panel_v1.5

| | Rel. Exp. (%) |
|---|---|
| | Ag5231, Run |
| Tissue Name | 229385251 |
| Adipose | 0.0 |
| Melanoma* Hs688(A).T | 0.0 |
| Melanoma* HS688(B).T | 0.0 |
| Melanoma* M14 | 0.0 |
| Melanoma* LOXIMVI | 0.0 |
| Melanoma* SK-MEL-5 | 2.9 |
| Squamous cell carcinoma SCC-4 | 0.0 |
| Testis Pool | 0.0 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 42.6 |
| Ovary | 0.0 |
| Breast ca. MCF-7 | 10.9 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 14.3 |
| Lung | 0.0 |
| Fetal Lung | 0.0 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 0.0 |
| Lung ca. NCI-H526 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 5.6 |
| Fetal Liver | 8.4 |
| Liver ca. HepG2 | 73.7 |
| Kidney Pool | 0.0 |
| Fetal Kidney | 0.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 25.9 |
| Bladder | 100.0 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 1.9 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.0 |
| Stomach Pool | 0.0 |
| Bone Marrow Pool | 0.0 |
| Fetal Heart | 0.0 |
| Heart Pool | 0.0 |
| Lymph Node Pool | 0.0 |
| Fetal Skeletal Muscle | 0.0 |
| Skeletal Muscle Pool | 3.0 |
| Spleen Pool | 0.0 |
| Thymus Pool | 0.0 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 3.2 |
| Brain (Amygdala) Pool | 0.0 |
| Brain (cerebellum) | 0.0 |
| Brain (fetal) | 0.0 |
| Brain (Hippocampus) Pool | 0.0 |
| Cerebral Cortex Pool | 0.0 |
| Brain (Substantia nigra) Pool | 0.0 |
| Brain (Thalamus) Pool | 0.0 |
| Brain (whole) | 3.7 |
| Spinal Cord Pool | 7.6 |
| Adrenal Gland | 0.0 |
| Pituitary gland Pool | 0.0 |
| Salivary Gland | 0.0 |
| Thyroid (female) | 0.0 |
| Pancreatic ca. CAPAN2 | 0.0 |
| Pancreas Pool | 33.9 |

CNS_Neurodegeneration_v1.0 Summary:

Ag5231 Expression of this gene is low/undetectable in all samples on this panel (CTs>35).

General_screening_panel_v1.5 Summary:

Ag5231 Expression of this gene is restricted to a sample derived from a liver cancer cell line and normal bladder (CT=34.2-34.6). Thus, expression of this gene could be used to differentiate between this sample and other samples on this panel and as a marker to detect the presence of liver cancer. Furthermore, therapeutic modulation of the expression or function of this gene may be effective in the treatment of liver cancer.

Panel 4.1D Summary:

Ag5231 Expression of this gene is low/undetectable in all samples on this panel (CTs>35).

P. CG57094-01: PPAR-Gamma.

Expression of gene CG57094-01 was assessed using the primer-probe sets Ag2012 and Ag383, described in Tables PA and PB. Results of the RTQ-PCR runs are shown in Tables PC, PD, PE, PF, PG, PH, PI, PJ and PK.

TABLE PA

Probe Name Ag2012

| | | | Start | SEQ ID |
|---|---|---|---|---|
| Primers | Sequence | Length | Position | No |
| Forward | 5′-aaggctcagaacagcaggat-3′ | 20 | 478 | 346 |
| Probe | TET-5′-caactcttccacaaggtggcccag-3′-TAMRA | 24 | 502 | 347 |
| Reverse | 5′-gctttgcagatgctgaattc-3′ | 20 | 557 | 348 |

TABLE PB

Probe Name Ag383

| | | | Start | SEQ ID |
|---|---|---|---|---|
| Primers | Sequence | Length | Position | No |
| Forward | 5′-ggcctctccgtacccttctc-3′ | 20 | 1111 | 349 |
| Probe | TET-5′-accaggatcacgacctccgcagg-3′-TAMRA | 23 | 1139 | 350 |
| Reverse | 5′-agaggctcttggcgcagtt-3′ | 19 | 1168 | 351 |

TABLE PC
AI comprehensive panel v1.0

| | Rel. Exp. (%) |
|---|---|
| | Ag2012, Run |
| Tissue Name | 228059650 |
| 110967 COPD-F | 3.3 |
| 110980 COPD-F | 1.7 |
| 110968 COPD-M | 3.6 |
| 110977 COPD-M | 3.9 |
| 110989 Emphysema-F | 2.7 |
| 110992 Emphysema-F | 1.2 |
| 110993 Emphysema-F | 2.1 |
| 110994 Emphysema-F | 1.7 |
| 110995 Emphysema-F | 1.9 |
| 110996 Emphysema-F | 0.3 |
| 110997 Asthma-M | 0.8 |
| 111001 Asthma-F | 0.7 |
| 111002 Asthma-F | 1.3 |
| 111003 Atopic Asthma-F | 2.5 |
| 111004 Atopic Asthma-F | 2.5 |
| 111005 Atopic Asthma-F | 1.4 |
| 111006 Atopic Asthma-F | 0.5 |
| 111417 Allergy-M | 0.9 |
| 112347 Allergy-M | 0.1 |
| 112349 Normal Lung-F | 0.0 |
| 112357 Normal Lung-F | 5.5 |
| 112354 Normal Lung-M | 1.1 |
| 112374 Crohns-F | 0.9 |
| 112389 Match Control Crohns-F | 1.9 |
| 112375 Crohns-F | 1.0 |
| 112732 Match Control Crohns-F | 2.4 |
| 112725 Crohns-M | 0.1 |
| 112387 Match Control Crohns-M | 0.7 |
| 112378 Crohns-M | 0.1 |
| 112390 Match Control Crohns-M | 1.9 |
| 112726 Crohns-M | 3.2 |
| 112731 Match Control Crohns-M | 1.1 |
| 112380 Ulcer Col-F | 3.4 |
| 112734 Match Control Ulcer Col-F | 4.6 |
| 112384 Ulcer Col-F | 4.3 |
| 112737 Match Control Ulcer Col-F | 1.4 |
| 112386 Ulcer Col-F | 1.2 |
| 112738 Match Control Ulcer Col-F | 2.2 |
| 112381 Ulcer Col-M | 0.2 |
| 112735 Match Control Ulcer Col-M | 0.4 |
| 112382 Ulcer Col-M | 2.2 |
| 112394 Match Control Ulcer Col-M | 0.3 |
| 112383 Ulcer Col-M | 1.5 |
| 112736 Match Control Ulcer Col-M | 1.7 |
| 112423 Psoriasis-F | 1.8 |
| 112427 Match Control Psoriasis-F | 3.9 |
| 112418 Psoriasis-M | 3.2 |
| 112723 Match Control Psoriasis-M | 5.1 |
| 112419 Psoriasis-M | 3.1 |
| 112424 Match Control Psoriasis-M | 0.9 |
| 112420 Psoriasis-M | 7.2 |
| 112425 Match Control Psoriasis-M | 2.2 |
| 104689 (MF) OA Bone-Backus | 58.2 |
| 104690 (MF) Adj “Normal” Bone-Backus | 72.2 |
| 104691 (MF) OA Synovium-Backus | 29.3 |
| 104692 (BA) OA Cartilage-Backus | 81.8 |
| 104694 (BA) OA Bone-Backus | 13.6 |
| 104695 (BA) Adj “Normal” Bone-Backus | 48.6 |
| 104696 (BA) OA Synovium-Backus | 37.4 |
| 104700 (SS) OA Bone-Backus | 36.9 |
| 104701 (SS) Adj “Normal” Bone-Backus | 42.0 |
| 104702 (SS) OA Synovium-Backus | 100.0 |
| 117093 OA Cartilage Rep7 | 4.2 |
| 112672 OA Bone5 | 4.5 |
| 112673 OA Synovium5 | 1.6 |
| 112674 OA Synovial Fluid cells5 | 2.0 |
| 117100 OA Cartilage Rep14 | 1.9 |
| 112756 OA Bone9 | 1.8 |
| 112757 OA Synovium9 | 4.5 |
| 112758 OA Synovial Fluid Cells9 | 1.7 |
| 117125 RA Cartilage Rep2 | 13.6 |
| 113492 Bone2 RA | 3.0 |
| 113493 Synovium2 RA | 1.0 |
| 113494 Syn Fluid Cells RA | 2.8 |
| 113499 Cartilage4 RA | 2.4 |
| 113500 Bone4 RA | 2.6 |
| 113501 Synovium4 RA | 1.8 |
| 113502 Syn Fluid Cells4 RA | 1.5 |
| 113495 Cartilage3 RA | 1.8 |
| 113496 Bone3 RA | 1.7 |
| 113497 Synovium3 RA | 0.8 |
| 113498 Syn Fluid Cells3 RA | 3.0 |
| 117106 Normal Cartilage Rep20 | 7.3 |
| 113663 Bone3 Normal | 0.2 |
| 113664 Synovium3 Normal | 0.0 |
| 113665 Syn Fluid Cells3 Normal | 0.0 |
| 117107 Normal Cartilage Rep22 | 1.5 |
| 113667 Bone4 Normal | 0.8 |
| 113668 Synovium4 Normal | 0.8 |
| 113669 Syn Fluid Cells4 Normal | 1.7 |

TABLE PD
Ardais Panel 1.1

| | Rel. Exp. (%) |
|---|---|
| | Ag2012, Run |
| Tissue Name | 315974369 |
| Lung adenocarcinoma SI A | 88.9 |
| Lung adenocarcinoma SI B | 54.3 |
| Lung adenocarcinoma SI B NAT | 18.9 |
| Lung adenocarcinoma SI C | 4.2 |
| Lung adenocarcinoma SI C NAT | 18.2 |
| Lung adenocarcinoma SII A | 22.7 |
| Lung adenocarcinoma SII A NAT | 28.5 |
| Lung adenocarcinoma SII C NAT | 44.8 |
| Lung adenocarcinoma SIII A | 100.0 |
| Lung adenocarcinoma SIII B | 15.1 |
| Lung adenocarcinoma SIII C | 33.9 |
| Lung SCC SI A | 11.4 |
| Lung SCC SI B NAT | 14.3 |
| Lung SCC SI C | 22.2 |
| Lung SCC SI C NAT | 65.5 |
| Lung SCC SI D | 73.2 |
| Lung SCC SI D NAT | 2.2 |
| Lung SCC SII A | 40.3 |
| Lung SCC SII B | 6.1 |
| Lung SCC SIII A | 7.6 |
| Lung SCC SIII A NAT | 4.4 |

TABLE PE
CNS neurodegeneration v1.0

| | Rel. Exp. (%) |
|---|---|
| | Ag2012, Run |
| Tissue Name | 207794919 |
| AD 1 Hippo | 28.3 |
| AD 2 Hippo | 39.0 |
| AD 3 Hippo | 9.9 |
| AD 4 Hippo | 16.3 |
| AD 5 Hippo | 55.1 |
| AD 6 Hippo | 100.0 |
| Control 2 Hippo | 45.7 |
| Control 4 Hippo | 15.8 |
| Control (Path) 3 Hippo | 18.4 |
| AD 1 Temporal Ctx | 32.1 |
| AD 2 Temporal Ctx | 37.6 |
| AD 3 Temporal Ctx | 12.4 |
| AD 4 Temporal Ctx | 18.7 |
| AD 5 Inf Temporal Ctx | 72.7 |
| AD 5 Sup Temporal Ctx | 62.9 |
| AD 6 Inf Temporal Ctx | 51.4 |
| AD 6 Sup Temporal Ctx | 52.5 |
| Control 1 Temporal Ctx | 8.2 |
| Control 2 Temporal Ctx | 26.2 |
| Control 3 Temporal Ctx | 47.3 |
| Control 3 Temporal Ctx | 10.3 |
| Control (Path) 1 Temporal Ctx | 10.6 |
| Control (Path) 2 Temporal Ctx | 13.1 |
| Control (Path) 3 Temporal Ctx | 29.9 |
| Control (Path) 4 Temporal Ctx | 15.7 |
| AD 1 Occipital Ctx | 17.9 |
| AD 2 Occipital Ctx (Missing) | 0.0 |
| AD 3 Occipital Ctx | 7.9 |
| AD 4 Occipital Ctx | 14.7 |
| AD 5 Occipital Ctx | 28.7 |
| AD 6 Occipital Ctx | 36.9 |
| Control 1 Occipital Ctx | 7.2 |
| Control 2 Occipital Ctx | 32.3 |
| Control 3 Occipital Ctx | 46.0 |
| Control 4 Occipital Ctx | 11.6 |
| Control (Path) 1 Occipital Ctx | 15.8 |
| Control (Path) 2 Occipital Ctx | 5.0 |
| Control (Path) 3 Occipital Ctx | 12.3 |
| Control (Path) 4 Occipital Ctx | 6.5 |
| Control 1 Parietal Ctx | 8.5 |
| Control 2 Parietal Ctx | 57.0 |
| Control 3 Parietal Ctx | 29.7 |
| Control (Path) 1 Parietal Ctx | 8.2 |
| Control (Path) 2 Parietal Ctx | 10.6 |
| Control (Path) 3 Parietal Ctx | 25.9 |
| Control (Path) 4 Parietal Ctx | 18.6 |

TABLE PF
Panel 1

| | Rel. Exp. (%) |
|---|---|
| | Ag383, Run |
| Tissue Name | 109660410 |
| Endothelial cells | 3.5 |
| Endothelial cells (treated) | 2.9 |
| Pancreas | 9.4 |
| Pancreatic ca. CAPAN 2 | 3.7 |
| Adrenal gland | 18.0 |
| Thyroid | 13.8 |
| Salivary gland | 0.0 |
| Pituitary gland | 2.2 |
| Brain (fetal) | 3.1 |
| Brain (whole) | 4.4 |
| Brain (amygdala) | 17.2 |
| Brain (cerebellum) | 1.6 |
| Brain (hippocampus) | 9.3 |
| Brain (substantia nigra) | 33.2 |
| Brain (thalamus) | 22.7 |
| Brain (hypothalamus) | 5.7 |
| Spinal cord | 21.8 |
| glio/astro U87-MG | 2.2 |
| glio/astro U-118-MG | 4.5 |
| astrocytoma SW1783 | 0.0 |
| neuro*; met SK-N-AS | 2.7 |
| astrocytoma SF-539 | 0.2 |
| astrocytoma SNB-75 | 1.3 |
| glioma SNB-19 | 0.6 |
| glioma U251 | 0.2 |
| glioma SF-295 | 6.2 |
| Heart | 10.7 |
| Skeletal muscle | 18.4 |
| Bone marrow | 11.1 |
| Thymus | 7.3 |
| Spleen | 2.9 |
| Lymph node | 4.3 |
| Colon (ascending) | 1.3 |
| Stomach | 5.4 |
| Small intestine | 7.0 |
| Colon ca. SW480 | 0.4 |
| Colon ca.* SW620 (SW480 met) | 0.1 |
| Colon ca. HT29 | 0.4 |
| Colon ca. HCT-116 | 4.4 |
| Colon ca. CaCo-2 | 1.1 |
| Colon ca. HCT-15 | 11.0 |
| Colon ca. HCC-2998 | 0.0 |
| Gastric ca.* (liver met) NCI-N87 | 4.9 |
| Bladder | 18.8 |
| Trachea | 4.8 |
| Kidney | 7.3 |
| Kidney (fetal) | 11.0 |
| Renal ca. 786-0 | 0.4 |
| Renal ca. A498 | 56.3 |
| Renal ca. RXF 393 | 2.7 |
| Renal ca. ACHN | 1.0 |
| Renal ca. UO-31 | 1.8 |
| Renal ca. TK-10 | 13.4 |
| Liver | 74.7 |
| Liver (fetal) | 27.7 |
| Liver ca. (hepatoblast) HepG2 | 7.4 |
| Lung | 9.9 |
| Lung (fetal) | 1.5 |
| Lung ca. (small cell) LX-1 | 0.4 |
| Lung ca. (small cell) NCI-H69 | 0.5 |
| Lung ca. (s. cell var.) SHP-77 | 0.6 |
| Lung ca. (large cell)NCI-H460 | 20.6 |
| Lung ca. (non-sm. cell) A549 | 3.3 |
| Lung ca. (non-s. cell) NCI-H23 | 7.4 |
| Lung ca. (non-s. cell) HOP-62 | 32.1 |
| Lung ca. (non-s. cl) NCI-H522 | 11.0 |
| Lung ca. (squam.) SW 900 | 3.3 |
| Lung ca. (squam.) NCI-H596 | 0.5 |
| Mammary gland | 30.4 |
| Breast ca.* (pl. ef) MCF-7 | 4.8 |
| Breast ca.* (pl. ef) MDA-MB-231 | 2.2 |
| Breast ca.* (pl. ef) T47D | 9.8 |
| Breast ca. BT-549 | 9.2 |
| Breast ca. MDA-N | 1.3 |
| Ovary | 6.0 |
| Ovarian ca. OVCAR-3 | 1.6 |
| Ovarian ca. OVCAR-4 | 1.9 |
| Ovarian ca. OVCAR-5 | 7.1 |
| Ovarian ca. OVCAR-8 | 1.3 |
| Ovarian ca. IGROV-1 | 0.7 |
| Ovarian ca. (ascites) SK-OV-3 | 2.5 |
| Uterus | 6.3 |
| Placenta | 100.0 |
| Prostate | 13.3 |
| Prostate ca.* (bone met) PC-3 | 7.9 |
| Testis | 14.3 |
| Melanoma Hs688(A).T | 1.4 |
| Melanoma* (met) Hs688(B).T | 5.3 |
| Melanoma UACC-62 | 0.6 |
| Melanoma M14 | 0.9 |
| Melanoma LOX IMVI | 1.0 |
| Melanoma* (met) SK-MEL-5 | 0.0 |
| Melanoma SK-MEL-28 | 1.7 |

TABLE PG
Panel 1.3D

| | Rel. Exp. (%) | Rel. Exp. (%) |
|---|---|---|
| | g2012, | Ag2012, |
| | Run | Run |
| Tissue Name | 147816240 | 165526994 |
| Liver adenocarcinoma | 26.2 | 37.6 |
| Pancreas | 4.1 | 3.6 |
| Pancreatic ca. CAPAN2 | 3.4 | 4.9 |
| Adrenal gland | 11.2 | 15.7 |
| Thyroid | 13.9 | 11.7 |
| Salivary gland | 2.9 | 5.4 |
| Pituitary gland | 2.7 | 3.5 |
| Brain (fetal) | 4.4 | 12.9 |
| Brain (whole) | 11.1 | 21.0 |
| Brain (amygdala) | 7.3 | 18.7 |
| Brain (cerebellum) | 0.9 | 8.2 |
| Brain (hippocampus) | 21.0 | 31.6 |
| Brain (substantia nigra) | 4.0 | 17.4 |
| Brain (thalamus) | 8.0 | 22.7 |
| Cerebral Cortex | 22.8 | 16.8 |
| Spinal cord | 17.1 | 37.6 |
| glio/astro U87-MG | 2.7 | 2.3 |
| glio/astro U-118-MG | 38.2 | 34.4 |
| astrocytoma SW1783 | 20.2 | 27.9 |
| neuro*; met SK-N-AS | 10.7 | 5.1 |
| astrocytoma SF-539 | 0.3 | 0.6 |
| astrocytoma SNB-75 | 15.7 | 5.2 |
| glioma SNB-19 | 0.0 | 1.0 |
| glioma U251 | 0.1 | 0.8 |
| glioma SF-295 | 4.3 | 2.5 |
| Heart (fetal) | 10.0 | 1.7 |
| Heart | 2.9 | 8.4 |
| Skeletal muscle (fetal) | 44.8 | 5.3 |
| Skeletal muscle | 2.2 | 14.6 |
| Bone marrow | 6.7 | 10.2 |
| Thymus | 3.7 | 3.8 |
| Spleen | 4.9 | 9.6 |
| Lymph node | 6.4 | 17.2 |
| Colorectal | 3.9 | 2.3 |
| Stomach | 5.7 | 7.6 |
| Small intestine | 5.3 | 13.6 |
| Colon ca. SW480 | 1.3 | 0.2 |
| Colon ca.* SW620(SW480 met) | 0.2 | 0.0 |
| Colon ca. HT29 | 0.6 | 0.1 |
| Colon ca. HCT-116 | 2.6 | 4.6 |
| Colon ca. CaCo-2 | 0.8 | 0.5 |
| Colon ca. tissue(ODO3866) | 23.7 | 15.3 |
| Colon ca. HCC-2998 | 3.9 | 1.8 |
| Gastric ca.* (liver met) NCI-N87 | 6.6 | 8.7 |
| Bladder | 6.0 | 11.9 |
| Trachea | 6.1 | 13.1 |
| Kidney | 0.4 | 1.0 |
| Kidney (fetal) | 22.1 | 29.5 |
| Renal ca. 786-0 | 0.1 | 0.0 |
| Renal ca. A498 | 100.0 | 73.7 |
| Renal ca. RXF 393 | 4.8 | 10.9 |
| Renal ca. ACHN | 3.5 | 1.9 |
| Renal ca. UO-31 | 2.0 | 1.8 |
| Renal ca. TK-10 | 3.3 | 4.1 |
| Liver | 8.7 | 31.4 |
| Liver (fetal) | 12.0 | 16.4 |
| Liver ca. (hepatoblast) HepG2 | 5.7 | 4.0 |
| Lung | 18.7 | 28.5 |
| Lung (fetal) | 4.4 | 0.9 |
| Lung ca. (small cell) LX-1 | 0.6 | 0.9 |
| Lung ca. (small cell) NCI-H69 | 0.6 | 0.0 |
| Lung ca. (s. cell var.) SHP-77 | 1.0 | 0.3 |
| Lung ca. (large cell)NCI-H460 | 1.8 | 10.4 |
| Lung ca. (non-sm. cell) A549 | 3.1 | 2.5 |
| Lung ca. (non-s. cell) NCI-H23 | 6.3 | 4.0 |
| Lung ca. (non-s. cell) HOP-62 | 23.7 | 29.1 |
| Lung ca. (non-s. cl) NCI-H522 | 10.8 | 8.2 |
| Lung ca. (squam.) SW900 | 1.2 | 1.2 |
| Lung ca. (squam.) NCI-H596 | 0.0 | 0.3 |
| Mammary gland | 35.1 | 16.8 |
| Breast ca.* (pl. ef) MCF-7 | 2.6 | 4.5 |
| Breast ca.* (pl. ef) MDA-MB-231 | 6.3 | 8.5 |
| Breast ca.* (pl. ef) T47D | 8.0 | 6.8 |
| Breast ca. BT-549 | 40.6 | 43.5 |
| Breast ca. MDA-N | 0.8 | 0.2 |
| Ovary | 14.1 | 4.9 |
| Ovarian ca. OVCAR-3 | 0.4 | 0.9 |
| Ovarian ca. OVCAR-4 | 1.3 | 3.1 |
| Ovarian ca. OVCAR-5 | 6.2 | 5.6 |
| Ovarian ca. OVCAR-8 | 0.3 | 0.0 |
| Ovarian ca. IGROV-1 | 0.0 | 0.2 |
| Ovarian ca.* (ascites) SK-OV-3 | 3.5 | 3.3 |
| Uterus | 4.5 | 6.5 |
| Placenta | 95.9 | 94.6 |
| Prostate | 9.3 | 26.6 |
| Prostate ca.* (bone met)PC-3 | 2.7 | 3.1 |
| Testis | 2.9 | 4.0 |
| Melanoma Hs688(A).T | 4.4 | 2.4 |
| Melanoma* (met) Hs688(B).T | 27.7 | 4.5 |
| Melanoma UACC-62 | 0.2 | 1.2 |
| Melanoma M14 | 0.0 | 3.1 |
| Melanoma LOXIMVI | 1.2 | 0.1 |
| Melanoma* (met) SK-MEL-5 | 0.0 | 0.0 |
| Adipose | 59.9 | 100.0 |

TABLE PH
Panel 2D

| | Rel. Exp. (%) | Rel. Exp. (%) |
|---|---|---|
| | Ag2012, Run | Ag2012, Run |
| Tissue Name | 155560760 | 164981025 |
| Normal Colon | 1.0 | 1.0 |
| CC Well to Mod Diff | 0.9 | 0.9 |
| (ODO3866) | | |
| CC Margin | 0.6 | 0.3 |
| (ODO3866) | | |
| CC Gr.2 | 0.5 | 0.5 |
| rectosigmoid | | |
| (ODO3868) | | |
| CC Margin | 0.4 | 0.2 |
| (ODO3868) | | |
| CC Mod Diff | 0.1 | 0.1 |
| (ODO3920) | | |
| CC Margin | 0.3 | 0.4 |
| (ODO3920) | | |
| CC Gr.2 ascend colon | 0.2 | 0.3 |
| (ODO3921) | | |
| CC Margin | 0.2 | 0.2 |
| (ODO3921) | | |
| CC from Partial | 1.6 | 1.8 |
| Hepatectomy | | |
| (ODO4309) Mets | | |
| Liver Margin | 1.8 | 2.0 |
| (ODO4309) | | |
| Colon mets to lung | 0.5 | 0.7 |
| (OD04451-01) | | |
| Lung Margin | 0.2 | 0.2 |
| (OD04451-02) | | |
| Normal Prostate | 1.1 | 3.5 |
| 6546-1 | | |
| Prostate Cancer | 0.2 | 0.2 |
| (OD04410) | | |
| Prostate Margin | 0.5 | 0.4 |
| (OD04410) | | |
| Prostate Cancer | 0.3 | 0.3 |
| (OD04720-01) | | |
| Prostate Margin | 0.4 | 0.5 |
| (OD04720-02) | | |
| Normal Lung 061010 | 0.3 | 0.4 |
| Lung Met to Muscle | 1.6 | 1.9 |
| (ODO4286) | | |
| Muscle Margin | 4.2 | 6.0 |
| (ODO4286) | | |
| Lung Malignant | 0.4 | 0.4 |
| Cancer (OD03126) | | |
| Lung Margin | 0.3 | 0.2 |
| (OD03126) | | |
| Lung Cancer | 2.5 | 3.3 |
| (OD04404) | | |
| Lung Margin | 1.7 | 1.7 |
| (OD04404) | | |
| Lung Cancer | 0.3 | 0.2 |
| (OD04565) | | |
| Lung Margin | 0.2 | 0.2 |
| (OD04565) | | |
| Lung Cancer | 0.6 | 0.5 |
| (OD04237-01) | | |
| Lung Margin | 4.6 | 5.2 |
| (OD04237-02) | | |
| Ocular Mel Met to | 0.0 | 0.0 |
| Liver (ODO4310) | | |
| Liver Margin | 2.5 | 3.2 |
| (ODO4310) | | |
| Melanoma Mets to | 0.6 | 0.9 |
| Lung (OD04321) | | |
| Lung Margin | 1.6 | 1.4 |
| (OD04321) | | |
| Normal Kidney | 0.1 | 0.1 |
| Kidney Ca, Nuclear | 0.5 | 0.3 |
| grade 2 (OD04338) | | |
| Kidney Margin | 0.9 | 1.2 |
| (OD04338) | | |
| Kidney Ca Nuclear | 1.2 | 1.2 |
| grade 1/2 (OD04339) | | |
| Kidney Margin | 1.3 | 1.0 |
| (OD04339) | | |
| Kidney Ca, Clear cell | 100.0 | 100.0 |
| type (OD04340) | | |
| Kidney Margin | 0.9 | 1.1 |
| (OD04340) | | |
| Kidney Ca, Nuclear | 8.1 | 9.3 |
| grade 3 (OD04348) | | |
| Kidney Margin | 0.6 | 0.8 |
| (OD04348) | | |
| Kidney Cancer | 32.3 | 53.6 |
| (OD04622-01) | | |
| Kidney Margin | 0.2 | 0.3 |
| (OD04622-03) | | |
| Kidney Cancer | 0.2 | 0.2 |
| (OD04450-01) | | |
| Kidney Margin | 0.2 | 0.3 |
| (OD04450-03) | | |
| Kidney Cancer | 0.2 | 0.2 |
| 8120607 | | |
| Kidney Margin | 0.1 | 0.1 |
| 8120608 | | |
| Kidney Cancer | 0.1 | 0.3 |
| 8120613 | | |
| Kidney Margin | 0.9 | 1.0 |
| 8120614 | | |
| Kidney Cancer | 24.7 | 26.8 |
| 9010320 | | |
| Kidney Margin | 1.1 | 1.1 |
| 9010321 | | |
| Normal Uterus | 0.1 | 0.1 |
| Uterus Cancer | 0.5 | 0.4 |
| 064011 | | |
| Normal Thyroid | 1.1 | 0.6 |
| Thyroid Cancer | 1.4 | 2.0 |
| 064010 | | |
| Thyroid Cancer | 0.2 | 0.2 |
| A302152 | | |
| Thyroid Margin | 0.5 | 0.3 |
| A302153 | | |
| Normal Breast | 0.9 | 0.9 |
| Breast Cancer | 0.2 | 0.2 |
| (OD04566) | | |
| Breast Cancer | 0.4 | 0.6 |
| (OD04590-01) | | |
| Breast Cancer | 2.0 | 1.7 |
| Mets | | |
| (OD04590-03) | | |
| Breast Cancer | 0.5 | 0.2 |
| Metastasis | | |
| (OD04655-05) | | |
| Breast Cancer | 0.2 | 0.2 |
| 064006 | | |
| Breast Cancer | 0.7 | 0.6 |
| 1024 | | |
| Breast Cancer | 0.5 | 0.4 |
| 9100266 | | |
| Breast Margin | 0.5 | 0.6 |
| 9100265 | | |
| Breast Cancer | 0.4 | 0.4 |
| A209073 | | |
| Breast Margin | 0.3 | 0.3 |
| A209073 | | |
| Normal Liver | 1.8 | 2.8 |
| Liver Cancer | 4.2 | 3.4 |
| 064003 | | |
| Liver Cancer 1025 | 3.6 | 3.8 |
| Liver Cancer 1026 | 2.0 | 2.8 |
| Liver Cancer | 7.1 | 4.8 |
| 6004-T | | |
| Liver Tissue | 0.9 | 1.1 |
| 6004-N | | |
| Liver Cancer | 2.8 | 2.3 |
| 6005-T | | |
| Liver Tissue | 1.4 | 1.7 |
| 6005-N | | |
| Normal Bladder | 2.3 | 1.8 |
| Bladder Cancer | 0.7 | 0.6 |
| 1023 | | |
| Bladder Cancer | 0.4 | 0.4 |
| A302173 | | |
| Bladder Cancer | 2.9 | 2.6 |
| (OD04718-01) | | |
| Bladder Normal | 2.7 | 2.9 |
| Adjacent | | |
| (OD04718-03) | | |
| Normal Ovary | 0.4 | 0.3 |
| Ovarian Cancer | 0.3 | 0.3 |
| 064008 | | |
| Ovarian Cancer | 4.1 | 6.0 |
| (OD04768-07) | | |
| Ovary Margin | 3.1 | 2.6 |
| (OD04768-08) | | |
| Normal Stomach | 0.3 | 0.3 |
| Gastric Cancer | 0.1 | 0.1 |
| 9060358 | | |
| Stomach Margin | 0.1 | 0.2 |
| 9060359 | | |
| Gastric Cancer | 0.2 | 0.3 |
| 9060395 | | |
| Stomach Margin | 0.3 | 0.3 |
| 9060394 | | |
| Gastric Cancer | 0.1 | 0.2 |
| 9060397 | | |
| Stomach Margin | 0.2 | 0.2 |
| 9060396 | | |
| Gastric Cancer | 0.4 | 0.6 |
| 064005 | | |

TABLE PI
Panel 3D

| | Rel. Exp. (%) | Rel. Exp. (%) |
|---|---|---|
| | Ag2012, Run | Ag2012, Run |
| Tissue Name | 155560795 | 164165421 |
| Daoy- | 0.1 | 0.3 |
| Medulloblastoma | | |
| TE671- | 3.2 | 2.8 |
| Medulloblastoma | | |
| D283 Med- | 0.4 | 0.5 |
| Medulloblastoma | | |
| PFSK-1- Primitive | 3.1 | 2.7 |
| Neuroectodermal | | |
| XF-498- CNS | 3.1 | 2.5 |
| SNB-78- Glioma | 7.6 | 4.0 |
| SF-268- Glioblastoma | 2.9 | 1.9 |
| T98G- Glioblastoma | 0.2 | 0.6 |
| SK-N-SH- | 11.7 | 11.3 |
| Neuroblastoma | | |
| (metastasis) | | |
| SF-295- Glioblastoma | 1.4 | 0.9 |
| Cerebellum | 5.5 | 5.3 |
| Cerebellum | 3.3 | 3.0 |
| NCI-H292- | 8.2 | 7.9 |
| Mucoepidermoid lung | | |
| carcinoma | | |
| DMS-114- Small cell | 1.1 | 2.3 |
| lung cancer | | |
| DMS-79- Small cell | 8.5 | 9.7 |
| lung cancer | | |
| NCI-H146- Small cell | 0.6 | 1.0 |
| lung cancer | | |
| NCI-H526- Small cell | 0.8 | 1.3 |
| lung cancer | | |
| NCI-N417- Small cell | 0.0 | 0.4 |
| lung cancer | | |
| NCI-H82- Small cell | 0.3 | 0.0 |
| lung cancer | | |
| NCI-H157- Squamous | 0.2 | 0.9 |
| cell lung cancer | | |
| (metastasis) | | |
| NCI-H1155- Large | 0.5 | 0.5 |
| cell lung cancer | | |
| NCI-H1299- Large | 37.9 | 36.1 |
| cell lung cancer | | |
| NCI-H727- Lung | 0.7 | 1.0 |
| carcinoid | | |
| NCI-UMC-11- Lung | 0.7 | 0.4 |
| carcinoid | | |
| LX-1- Small cell lung | 0.0 | 0.3 |
| cancer | | |
| Colo-205- Colon | 0.8 | 0.4 |
| cancer | | |
| KM12- Colon cancer | 1.0 | 1.2 |
| KM20L2- Colon | 0.0 | 0.0 |
| cancer | | |
| NCI-H716- Colon | 6.1 | 8.4 |
| cancer | | |
| SW-48- Colon | 0.3 | 1.2 |
| adenocarcinoma | | |
| SW1116- Colon | 0.4 | 0.5 |
| adenocarcinoma | | |
| LS 174T- Colon | 0.2 | 0.4 |
| adenocarcinoma | | |
| SW-948- Colon | 0.0 | 0.2 |
| adenocarcinoma | | |
| SW-480- Colon | 0.5 | 0.2 |
| adenocarcinoma | | |
| NCI-SNU-5- Gastric | 1.5 | 1.3 |
| carcinoma | | |
| KATO III- Gastric | 1.2 | 5.9 |
| carcinoma | | |
| NCI-SNU-16- Gastric | 97.3 | 95.9 |
| carcinoma | | |
| NCI-SNU-1- Gastric | 1.4 | 1.0 |
| carcinoma | | |
| RF-1- Gastric | 0.0 | 0.3 |
| adenocarcinoma | | |
| RF-48- Gastric | 0.0 | 0.4 |
| adenocarcinoma | | |
| MKN-45- Gastric | 3.4 | 4.4 |
| carcinoma | | |
| NCI-N87- Gastric | 0.3 | 0.9 |
| carcinoma | | |
| OVCAR-5- Ovarian | 2.0 | 1.5 |
| carcinoma | | |
| RL95-2- Uterine | 1.7 | 2.7 |
| carcinoma | | |
| HelaS3- Cervical | 1.2 | 0.5 |
| adenocarcinoma | | |
| Ca Ski- Cervical | 5.5 | 6.2 |
| epidermoid carcinoma | | |
| (metastasis) | | |
| ES-2- Ovarian clear | 1.5 | 1.0 |
| cell carcinoma | | |
| Ramos- Stimulated | 0.0 | 0.0 |
| with PMA/ionomycin | | |
| 6 h | | |
| Ramos- Stimulated | 0.0 | 0.2 |
| with PMA/ionomycin | | |
| 14 h | | |
| MEG-01- Chronic | 0.8 | 1.2 |
| myelogenous leukemia | | |
| (megokaryoblast) | | |
| Raji- Burkitt's | 0.2 | 0.4 |
| lymphoma | | |
| Daudi- Burkitt's | 0.3 | 0.4 |
| lymphoma | | |
| U266- B-cell | 1.1 | 0.6 |
| plasmacytoma | | |
| CA46- Burkitt's | 0.0 | 0.4 |
| lymphoma | | |
| RL- non-Hodgkin's | 0.0 | 0.2 |
| B-cell lymphoma | | |
| JM1- pre-B-cell | 0.2 | 0.6 |
| lymphoma | | |
| Jurkat- T cell leukemia | 1.2 | 0.4 |
| TF-1- Erythroleukemia | 0.3 | 0.5 |
| HUT 78- T-cell | 0.6 | 1.6 |
| lymphoma | | |
| U937- Histiocytic | 0.4 | 0.4 |
| lymphoma | | |
| KU-812- Myelogenous | 0.5 | 0.4 |
| leukemia | | |
| 769-P- Clear cell renal | 3.1 | 2.2 |
| carcinoma | | |
| Caki-2- Clear cell renal | 35.8 | 33.7 |
| carcinoma | | |
| SW 839- Clear cell | 24.5 | 40.6 |
| renal carcinoma | | |
| G401- Wilms' tumor | 0.9 | 0.9 |
| Hs766T- Pancreatic | 18.3 | 25.9 |
| carcinoma (LN | | |
| metastasis) | | |
| CAPAN-1- Pancreatic | 2.6 | 2.5 |
| adenocarcinoma (liver | | |
| metastasis) | | |
| SU86.86- Pancreatic | 0.2 | 0.2 |
| carcinoma (liver | | |
| metastasis) | | |
| BxPC-3- Pancreatic | 5.8 | 5.4 |
| adenocarcinoma | | |
| HPAC- Pancreatic | 1.4 | 3.0 |
| adenocarcinoma | | |
| MIA PaCa-2- | 2.5 | 4.9 |
| Pancreatic carcinoma | | |
| CFPAC-1- Pancreatic | 3.0 | 2.3 |
| ductal adenocarcinoma | | |
| PANC-1- Pancreatic | 100.0 | 100.0 |
| epithelioid ductal | | |
| carcinoma | | |
| T24- Bladder carcinma | 49.0 | 67.4 |
| (transitional cell) | | |
| 5637- Bladder | 0.4 | 0.2 |
| carcinoma | | |
| HT-1197- Bladder | 5.2 | 7.6 |
| carcinoma | | |
| UM-UC-3- Bladder | 62.0 | 81.2 |
| carcinma (transitional cell) | | |
| A204- | 0.6 | 2.0 |
| Rhabdomyosarcoma | | |
| HT-1080- | 0.1 | 0.4 |
| Fibrosarcoma | | |
| MG-63- Osteosarcoma | 18.7 | 13.1 |
| SK-LMS-1- | 9.3 | 9.1 |
| Leiomyosarcoma | | |
| (vulva) | | |
| SJRH30- | 0.4 | 0.6 |
| Rhabdomyosarcoma | | |
| (met to bone marrow) | | |
| A431- Epidermoid | 0.4 | 0.9 |
| carcinoma | | |
| WM266-4- Melanoma | 18.2 | 25.2 |
| DU 145- Prostate | 0.3 | 0.1 |
| carcinoma (brain | | |
| metastasis) | | |
| MDA-MB-468- Breast | 0.0 | 0.2 |
| adenocarcinoma | | |
| SCC-4- Squamous cell | 0.0 | 0.2 |
| carcinoma of tongue | | |
| SCC-9- Squamous cell | 0.7 | 0.7 |
| carcinoma of tongue | | |
| SCC-15- Squamous | 0.0 | 0.0 |
| cell carcinoma of | | |
| tongue | | |
| CAL 27- Squamous | 0.0 | 0.5 |
| cell carcinoma of | | |
| tongue | | |

TABLE PJ
Panel 4D

| | Rel. Exp. (%) | Rel. Exp. (%) |
|---|---|---|
| | Ag2012, Run | Ag2012, Run |
| Tissue Name | 155560840 | 163582094 |
| Secondary Th1 act | 0.2 | 0.1 |
| Secondary Th2 act | 0.3 | 0.2 |
| Secondary Tr1 act | 0.6 | 0.1 |
| Secondary Th1 rest | 0.1 | 0.1 |
| Secondary Th2 rest | 0.0 | 0.0 |
| Secondary Tr1 rest | 0.1 | 0.1 |
| Primary Th1 act | 0.0 | 0.1 |
| Primary Th2 act | 0.0 | 0.1 |
| Primary Tr1 act | 0.1 | 0.1 |
| Primary Th1 rest | 0.1 | 0.1 |
| Primary Th2 rest | 0.1 | 0.1 |
| Primary Tr1 rest | 0.1 | 0.1 |
| CD45RA CD4 | 0.1 | 0.1 |
| lymphocyte act | | |
| CD45RO CD4 | 0.1 | 0.1 |
| lymphocyte act | | |
| CD8 lymphocyte act | 0.1 | 0.1 |
| Secondary CD8 | 0.3 | 0.1 |
| lymphocyte rest | | |
| Secondary CD8 | 0.2 | 0.2 |
| lymphocyte act | | |
| CD4 lymphocyte none | 0.0 | 0.0 |
| 2ry | 0.1 | 0.0 |
| Th1/Th2/Tr1_anti-CD95 | | |
| CH11 | | |
| LAK cells rest | 0.1 | 0.1 |
| LAK cells IL-2 | 0.5 | 0.1 |
| LAK cells IL-2 + IL-12 | 0.1 | 0.1 |
| LAK cells IL-2 + IFN | 0.1 | 0.1 |
| gamma | | |
| LAK cells IL-2 + IL-18 | 0.2 | 0.1 |
| LAK cells | 35.8 | 18.3 |
| PMA/ionomycin | | |
| NK Cells IL-2 rest | 0.1 | 0.1 |
| Two Way MLR 3 day | 0.2 | 0.1 |
| Two Way MLR 5 day | 0.0 | 0.0 |
| Two Way MLR 7 day | 0.1 | 0.1 |
| PBMC rest | 0.1 | 0.1 |
| PBMC PWM | 0.3 | 0.1 |
| PBMC PHA-L | 1.8 | 1.2 |
| Ramos (B cell) none | 0.0 | 0.0 |
| Ramos (B cell) | 0.1 | 0.2 |
| ionomycin | | |
| B lymphocytes PWM | 0.6 | 0.5 |
| B lymphocytes CD40L | 0.3 | 0.1 |
| and IL-4 | | |
| EOL-1 dbcAMP | 0.3 | 0.0 |
| EOL-1 dbcAMP | 0.3 | 0.2 |
| PMA/ionomycin | | |
| Dendritic cells none | 0.4 | 0.3 |
| Dendritic cells LPS | 0.8 | 0.9 |
| Dendritic cells | 0.3 | 0.3 |
| anti-CD40 | | |
| Monocytes rest | 0.2 | 0.1 |
| Monocytes LPS | 0.1 | 0.1 |
| Macrophages rest | 0.2 | 0.2 |
| Macrophages LPS | 0.5 | 0.3 |
| HUVEC none | 1.6 | 0.5 |
| HUVEC starved | 1.0 | 0.6 |
| HUVEC IL-1beta | 0.4 | 0.2 |
| HUVEC IFN gamma | 0.8 | 0.5 |
| HUVEC TNF alpha + | 0.5 | 0.6 |
| IFN gamma | | |
| HUVEC TNF alpha + | 3.0 | 1.9 |
| IL4 | | |
| HUVEC IL-11 | 1.2 | 0.7 |
| Lung Microvascular | 9.6 | 4.3 |
| EC none | | |
| Lung Microvascular | 11.0 | 5.8 |
| EC TNFalpha + | | |
| IL-1beta | | |
| Microvascular | 16.5 | 9.7 |
| Dermal EC none | | |
| Microsvasular | 9.9 | 6.7 |
| Dermal EC | | |
| TNFalpha + IL-1beta | | |
| Bronchial epithelium | 3.7 | 4.2 |
| TNFalpha + IL1beta | | |
| Small airway | 18.6 | 13.5 |
| epithelium none | | |
| Small airway | 100.0 | 100.0 |
| epithelium TNFalpha + | | |
| IL-1beta | | |
| Coronery artery SMC | 6.7 | 7.9 |
| rest | | |
| Coronery artery SMC | 2.1 | 2.0 |
| TNFalpha + IL-1beta | | |
| Astrocytes rest | 5.3 | 5.1 |
| Astrocytes TNFalpha + | 8.1 | 5.6 |
| IL-1beta | | |
| KU-812 (Basophil) | 0.0 | 0.1 |
| rest | | |
| KU-812 (Basophil) | 0.8 | 0.7 |
| PMA/ionomycin | | |
| CCD1106 | 1.8 | 1.3 |
| (Keratinocytes) none | | |
| CCD1106 | 2.1 | 2.0 |
| (Keratinocytes) | | |
| TNFalpha + IL-1beta | | |
| Liver cirrhosis | 7.3 | 6.9 |
| Lupus kidney | 0.1 | 0.1 |
| NCI-H292 none | 0.2 | 0.3 |
| NCI-H292 IL-4 | 1.0 | 0.8 |
| NCI-H292 IL-9 | 0.5 | 0.5 |
| NCI-H292 IL-13 | 0.5 | 0.4 |
| NCI-H292 IFN | 0.4 | 0.6 |
| gamma | | |
| HPAEC none | 4.8 | 3.9 |
| HPAEC TNF alpha + | 6.6 | 2.8 |
| IL-1 beta | | |
| Lung fibroblast none | 2.0 | 2.0 |
| Lung fibroblast TNF | 0.7 | 0.3 |
| alpha + IL-1 beta | | |
| Lung fibroblast IL-4 | 14.8 | 10.2 |
| Lung fibroblast IL-9 | 4.1 | 4.5 |
| Lung fibroblast IL-13 | 7.0 | 6.5 |
| Lung fibroblast IFN | 13.0 | 10.5 |
| gamma | | |
| Dermal fibroblast | 2.2 | 1.4 |
| CCD1070 rest | | |
| Dermal fibroblast | 1.0 | 1.3 |
| CCD1070 TNF alpha | | |
| Dermal fibroblast | 0.8 | 1.0 |
| CCD1070 IL-1 beta | | |
| Dermal fibroblast | 1.1 | 1.2 |
| IFN gamma | | |
| Dermal fibroblast | 7.9 | 7.5 |
| IL-4 | | |
| IBD Colitis 2 | 0.8 | 0.7 |
| IBD Crohn's | 2.4 | 2.4 |
| Colon | 3.8 | 2.1 |
| Lung | 4.6 | 6.9 |
| Thymus | 0.8 | 0.5 |
| Kidney | 3.6 | 2.0 |

TABLE PK
Panel 5 Islet

| | Rel. Exp. (%) |
|---|---|
| | Ag2012 Run |
| Tissue Name | 254275032 |
| 97457_Patient-02go_adipose | 8.5 |
| 97476_Patient-07sk_skeletal muscle | 5.7 |
| 97477_Patient-07ut_uterus | 1.7 |
| 97478_Patient-07pl_placenta | 50.7 |
| 99167_Bayer Patient 1 | 47.6 |
| 97482_Patient-08ut_uterus | 1.8 |
| 97483_Patient-08pl_placenta | 32.8 |
| 97486_Patient-09sk_skeletal muscle | 3.5 |
| 97487_Patient-09ut_uterus | 0.5 |
| 97488_Patient-09pl_placenta | 29.7 |
| 97492_Patient-10ut_uterus | 2.8 |
| 97493_Patient-1Opl_placenta | 74.2 |
| 97495_Patient-11go_adipose | 7.9 |
| 97496_Patient-11sk_skeletal muscle | 4.8 |
| 97497_Patient-11ut_uterus | 2.0 |
| 97498_Patient-11pl_placenta | 20.2 |
| 97500_Patient-12go_adipose | 16.4 |
| 9750l_Patient-12sk_skeletal muscle | 34.6 |
| 97502_Patient-12ut_uterus | 2.3 |
| 97503_Patient-12pl_placenta | 27.9 |
| 94721_Donor 2 U - A_Mesenchymal Stem Cells | 2.0 |
| 94722_Donor 2 U - B_Mesenchymal Stem Cells | 2.1 |
| 94723_Donor 2 U - C_Mesenchymal Stem Cells | 1.7 |
| 94709_Donor 2 AM - A_adipose | 21.6 |
| 94710_Donor 2 AM - B_adipose | 18.0 |
| 94711_Donor 2 AM - C_adipose | 13.9 |
| 94712_Donor 2 AD - A_adipose | 6.0 |
| 94713_Donor 2 AD - B_adipose | 9.2 |
| 94714_Donor 2 AD - C_adipose | 22.5 |
| 94742_Donor 3 U - A_Mesenchymal Stem Cells | 2.0 |
| 94743_Donor 3 U - B_Mesenchymal Stem Cells | 5.1 |
| 94730_Donor 3 AM - A_adipose | 100.0 |
| 94731_Donor 3 AM - B_adipose | 64.2 |
| 94732_Donor 3 AM - C_adipose | 49.3 |
| 94733_Donor 3 AD - A_adipose | 14.7 |
| 94734_Donor 3 AD - B_adipose | 9.4 |
| 94735_Donor 3 AD - C_adipose | 19.5 |
| 77138_Liver_HepG2untreated | 6.9 |
| 73556_Heart_Cardiac stromal cells (primary) | 4.4 |
| 81735_Small Intestine | 4.2 |
| 72409_Kidney_Proximal Convoluted Tubule | 5.1 |
| 82685_Small intestine_Duodenum | 5.9 |
| 90650_Adrenal_Adrenocortical adenoma | 2.6 |
| 72410_Kidney_HRCE | 49.0 |
| 72411_Kidney_HRE | 11.3 |
| 73139_Uterus_Uterine smooth muscle cells | 1.6 |

AI_Comprehensive Panel_v1.0 Summary:

Ag2012 This gene shows a wide spread expression in this panel, with moderate to low expression in samples derived from normal and orthoarthitis/rheumatoid arthritis bone and adjacent bone, cartilage, synovium and synovial fluid samples, from normal lung, COPD lung, emphysema, atopic asthma, asthma, allergy, Crohn's disease (normal matched control and diseased), ulcerative colitis(normal matched control and diseased), and psoriasis (normal matched control and diseased). This gene appears to be upregulated in samples of bone, cartilage and synovium from patients with osteorarthritis when compared to expression in corresponding normal samples. Thus, therapeutic modulation of the expression or function of this gene may be effective in the treatment of oseoarthritis.

Ardais Panel 1.1 Summary:

Ag2012 Highest expression of this gene is detected in lung cancer (358) sample (CT=26.6). This gene is expressed both in normal and cancer lung tissues. Higher expression of this gene is associated with the cancer as compared to normal lung. Therefore, expression of this gene may be used as a diagnostic marker for lung cancer and also, therapeutic modulation of this gene through the use of antibodies may be useful in the treatment of lung cancer.

CNS_Neurodegeneration_v1.0 Summary:

Ag2012 This gene is present in the brain as evidenced by expression in this panel and panel 1.3D. No apparent association with Alzheimer's disease is seen. However, Thus, therapeutic modulation of the expression or function of this gene may be effective in the treatment of neurologic diseases.

Panel 1 Summary:

Ag383 Highest expression of this gene is detected in placenta (CT=21.7). This gene shows a widespread expression in this panel, which corelates with the expression seen in panel 1.3D. Please see panel 1.3D for further discussion.

Panel 1.3D Summary:

Ag2012 Two experiments with same probe and primer sets are in good agreement. Highest expression of this gene is seen in a renal cancer cell line and adipose tissue (CTs=28.7-29). Significant expression is also seen in breast, brain, colon, liver, renal and melanoma cancer cell lines. Thus, expression of this gene could be used to differentiate between the lung cancer cell line and other samples on this panel and as a marker for these cancers. This gene is identical to angiopoeitin related protein 4 (ARP4), which is know to be angiogenic [1]. Since angiogenesis is essential for the growth and metastasis of solid tumors, therapeutic modulation of the expression or function of this ARP protein encoded by this gene, through the use of protein therapeutics or antibodies, may be effective in the treatment of melanoma, brain, colon, renal and liver cancers.

Among tissues with metabolic function, this gene is expressed most highly in adipose with moderate levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes. ARP4 has been widely studied in the context of adipose biology [2]. The mouse gene, known as fasting-induced adipose factor, is predominantly expressed in adipose tissue and is strongly upregulated by fasting in white adipose tissue and liver[3]. The N-terminal and C-terminal portions contain the characteristic coiled-coil domains and fibrinogen-like domains that are conserved in angiopoietins. In human and mouse tissues, it is specifically expressed in the liver and they are mainly present in the hepatocytes [4]. Recombinant protein expressed in COS-7 cells is secreted and glycosylated. Furthermore, Angiopoietin-2 has been implicated in adipose tissue regression [5]. Since this molecule is an angiopoietin homolog that is highly expressed in adipose, this molecule may also play a role in initiation of apoptosis in adipose. Thus, therapeutic modulation of the expression or function of this gene may be effective in the treatment of obesity.

In addition, expression of this gene is higher in fetal kidney (CTs=30-31) when compared to expression in adult kidney (CTs35-37). Thus, expression of this gene could be used to differentiate between adult and fetal kidney.

Furthermore, expression of this gene in fetal kidney and renal cell carcinoma-derived cell lines but not in adult kidney, suggests that it may be involved in kidney development and organogenesis and also, in kidney tumorgenesis.

References:

1. Kim I, Kim H G, Kim H, Kim H H, Park S K, Uhm C S, Lee Z H, Koh G Y. Hepatic expression, synthesis and secretion of a novel fibrinogen/angiopoietin-related protein that prevents endothelial-cell apoptosis. Biochem J Mar. 15, 2000;346 Pt 3:603-10. PMID: 10698685.

2. Yoon J C, Chickering T W, Rosen E D, Dussault B, Qin Y, Soukas A, Friedman J M, Holmes WE, Spiegelman B M. Peroxisome proliferator-activated receptor gamma target gene encoding a novel angiopoietin-related protein associated with adipose differentiation. Mol Cell Biol Jul. 20, 2000 (14):5343-9. PMID: 10866690

3. Kersten S, Mandard S, Tan N S, Escher P, Metzger D, Chambon P, Gonzalez F J, Desvergne B, Wahli W. Characterization of the fasting-induced adipose factor FIAF, a novel peroxisome proliferator-activated receptor target gene. J Biol Chem Sep. 15, 2000;275(37):28488-93. PMID: 10862772.

4. Reinmuth N, Stoeltzing O, Liu W, Ahmad S A, Jung Y D, Fan F, Parikh A, Ellis L M.Endothelial survival factors as targets for antineoplastic therapy. Cancer J 2001 Nov-Dec;7 Suppl 3:S109-19. PMID: 11779081

5. Cohen B, Barkan D, Levy Y, Goldberg I, Fridman E, Kopolovic J, Rubinstein M. Leptin induces angiopoietin-2 expression in adipose tissues. PMID: 11152449

Panel 2D Summary:

Ag2012 Two experiments with two different probe and primer sets produce results that are in excellent agreement, with highest expression in kidney cancer (CTs=22-24). Thus, expression of this gene could be used to differentiate between this sample and other samples on this panel and as a marker to detect the presence of kidney cancer. Furthermore, therapeutic modulation of the expression or function of ARP encoded by this gene through the use of protein therapeutics or antibodies, may be effective in the treatment of kidney cancer.

Panel 3D Summary:

Ag2012 Two experiments with two different probe and primer sets produce results that are in excellent agreement, with highest expression in gastric, bladder, renal, pancreatic, and lung cancer cell lines. Thus, expression of this gene could be used to differentiate between these samples and other samples on this panel.

Panel 4D Summary:

Ag2012 Two experiments with two different probe and primer sets produce results that are in excellent agreement, with highest expression in small airway epithelium treated with TNF-alpha and IL-1beta (CTs=24.4). Thus, expression of this gene could be used as a marker of activated epithelium. Interestingly, expression of this gene is upregulated upon immune-stimulation of the airway epithelial cells and lung fibroblasts by cytokines as compared to corresponding resting cells. Furthermore, expression of this gene in LAK cells treated with PMA/ionomycin is also upregulated relative to the expression in resting cells. These data indicate that ARP plays a role in inflammation related to the above cells of the pulmonary system and is thereby implicated as a target for therapeutic intervention by protein and antibody therapeutics, as well as, small molecule pharmaceuticals. A wholly human antibody directed at ARP, for example, may diminish the symptoms of patients with allergy, asthma or emphysema.

In addtion, the gene is expressed at significant levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel 5 Islet Summary:

Ag2012 Highest expression of this gene is detected in midway differentiated adipose tissue. This gene shows a wide spread expression in this panel, withmoderate expressions in adipose, placenta, skeletal muscle, uterus, kidney and small intestine. Interestingly, higher levels of expression of this gene is seen in midway differentiated adipose as compared to undifferentiated and differentiated adipose. Angiopoietin-related protein is shown to be associated with adipose differentiation. Therefore, therapeutic modulation of this gene or ARP encoded by this gene may be useful in the treatment of obesity and diabetes.

### Example D

### Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderbom et al., Determination of Single Nucleotide Polymorphisms by Real-time Pyrophosphate DNA Sequencing. Genome Research. 10 (8) 1249-1265, 2000).

Variants are reported individually but any combination of all or a select subset of variants are also included as contemplated NOVX embodiments of the invention.

TABLE D1
Variants of nucleotide sequence CG52113-01

| | Nucleotides | | | Amino Acids | | |
|---|---|---|---|---|---|---|
| Variant | Position | Initial | Modified | Position | Initial | Modified |
| 13378348 | 245 | G | C | 50 | Gln | His |
| 13381469 | 279 | T | C | 62 | Cys | Arg |
| 13373863 | 552 | G | A | 153 | Val | Ile |
| 13375571 | 657 | A | G | 188 | Asn | Asp |
| 13381468 | 737 | G | A | 214 | Leu | Leu |
| 13375570 | 796 | C | T | 234 | Pro | Leu |
| 13377895 | 808 | G | A | 238 | Ser | Asn |
| 13381465 | 1176 | G | A | N/A | N/A | N/A |

TABLE D2
Variants of nucleotide sequence CG103322-02

| | Nucleotides | | | Amino Acids | | |
|---|---|---|---|---|---|---|
| Variant | Position | Initial | Modified | Position | Initial | Modified |
| 13381406 | 355 | G | A | 116 | Glu | Glu |
| 13381407 | 361 | T | C | 118 | Gly | Gly |
| 13381410 | 691 | C | G | 228 | Val | Val |

TABLE D3
Variants of nucleotide sequence CG151575-02

| | Nucleotides | | | Amino Acids | | |
|---|---|---|---|---|---|---|
| Variant | Position | Initial | Modified | Position | Initial | Modified |
| 13381416 | 593 | G | A | 131 | Gly | Ser |
| 13381415 | 736 | C | A | 178 | Asp | Glu |

TABLE D4
Variants of nucleotide sequence CG152323-01

| | Nucleotides | | | Amino Acids | | |
|---|---|---|---|---|---|---|
| Variant | Position | Initial | Modified | Position | Initial | Modified |
| 13381423 | 162 | T | C | 28 | Cys | Arg |
| 13381425 | 1085 | T | C | 335 | Asn | Asn |
| 13381422 | 3011 | T | C | 977 | Asp | Asp |
| 13381421 | 3156 | G | A | 1026 | Ala | Thr |

TABLE D5
Variant of nucleotide sequence CG153011-01

| | Nucleotides | | | Amino Acids | | |
|---|---|---|---|---|---|---|
| Variant | Position | Initial | Modified | Position | Initial | Modified |
| 13381522 | 1268 | G | A | 326 | Gly | Arg |

TABLE D6
Variant of nucleotide sequence CG153042-01

| | Nucleotides | | | Amino Acids | | |
|---|---|---|---|---|---|---|
| Variant | Position | Initial | Modified | Position | Initial | Modified |
| 13381434 | 928 | C | T | 304 | Leu | Phe |

TABLE D7
Variant of nucleotide sequence CG153179-01

| | Nucleotides | | | Amino Acids | | |
|---|---|---|---|---|---|---|
| Variant | Position | Initial | Modified | Position | Initial | Modified |
| 13381427 | 241 | C | T | 65 | Ser | Ser |

TABLE D8
Variants of nucleotide sequence CG157760-02

| | Nucleotides | | | Amino Acids | | |
|---|---|---|---|---|---|---|
| Variant | Position | Initial | Modified | Position | Initial | Modified |
| 13381431 | 353 | G | A | 85 | Ser | Asn |
| 13381432 | 500 | C | T | 134 | Ala | Val |

TABLE D9
Variants of nucleotide sequence CG158114-01

| | Nucleotides | | | Amino Acids | | |
|---|---|---|---|---|---|---|
| Variant | Position | Initial | Modified | Position | Initial | Modified |
| 13381446 | 483 | C | T | 161 | Pro | Pro |
| 13381447 | 563 | T | C | 188 | Leu | Pro |
| 13381450 | 1698 | T | C | 566 | Asn | Asn |

TABLE D10
Variant of nucleotide sequence CG158553-01

| | Nucleotides | | | Amino Acids | | |
|---|---|---|---|---|---|---|
| Variant | Position | Initial | Modified | Position | Initial | Modified |
| 13381435 | 465 | T | C | 107 | Cys | Cys |

TABLE D11
Variants of nucleotide sequence CG158983-01

| | Nucleotides | | | Amino Acids | | |
|---|---|---|---|---|---|---|
| Variant | Position | Initial | Modified | Position | Initial | Modified |
| 13377605 | 130 | T | C | 38 | Pro | Pro |
| 13381442 | 154 | G | C | 46 | Thr | Thr |
| 13381443 | 514 | C | T | 166 | Ser | Ser |

TABLE D12
Variants of nucleotide sequence CG159015-01

| | Nucleotides | | | Amino Acids | | |
|---|---|---|---|---|---|---|
| Variant | Position | Initial | Modified | Position | Initial | Modified |
| 13381457 | 522 | T | C | 149 | Leu | Pro |
| 13381458 | 645 | C | T | 190 | Ser | Phe |
| 13381456 | 734 | G | A | 220 | Val | Ile |
| 13381460 | 801 | C | G | 242 | Ser | Cys |

TABLE D13
Variants of nucleotide sequence CG173007-01

| | Nucleotides | | | Amino Acids | | |
|---|---|---|---|---|---|---|
| Variant | Position | Initial | Modified | Position | Initial | Modified |
| 13381454 | 45 | T | A | N/A | N/A | N/A |
| 13381453 | 322 | A | G | 75 | Ile | Val |
| 13381452 | 1003 | T | C | 302 | Ser | Pro |
| 13381451 | 1697 | T | C | 533 | Leu | Pro |

TABLE D14
Variants of nucleotide sequence CG173357-01

| | Nucleotides | | | Amino Acids | | |
|---|---|---|---|---|---|---|
| Variant | Position | Initial | Modified | Position | Initial | Modified |
| 13381463 | 227 | T | C | 68 | Phe | Ser |
| 13381439 | 1877 | G | T | N/A | N/A | N/A |

TABLE D15
Variant of nucleotide sequence CG50387-03

| | Nucleotides | | | Amino Acids | | |
|---|---|---|---|---|---|---|
| Variant | Position | Initial | Modified | Position | Initial | Modified |
| 13377608 | 1017 | G | A | 339 | Ala | Ala |

TABLE D16
Variants of nucleotide sequence CG103134-02

| | Nucleotides | | | Amino Acids | | |
|---|---|---|---|---|---|---|
| Variant | Position | Initial | Modified | Position | Initial | Modified |
| 13381413 | 231 | C | T | 61 | His | Tyr |
| 13381402 | 478 | T | C | 143 | Val | Ala |
| 13381414 | 663 | G | C | 205 | Val | Leu |

TABLE D17
Variants of nucleotide sequence CG57542-01

| | Nucleotides | | | Amino Acids | | |
|---|---|---|---|---|---|---|
| Variant | Position | Initial | Modified | Position | Initial | Modified |
| 13377100 | 495 | C | T | 155 | Asp | Asp |
| 13377101 | 820 | G | A | 264 | Ala | Thr |

TABLE D18
Variants of nucleotide sequence CG57774-01

| | Nucleotides | | | Amino Acids | | |
|---|---|---|---|---|---|---|
| Variant | Position | Initial | Modified | Position | Initial | Modified |
| 13381484 | 640 | C | T | 180 | Pro | Pro |
| 13381486 | 698 | C | A | 200 | Pro | Thr |
| 13375028 | 1422 | T | C | 441 | Leu | Ser |

TABLE D19
Variant of nucleotide sequence CG89285-03

| | Nucleotides | | | Amino Acids | | |
|---|---|---|---|---|---|---|
| Variant | Position | Initial | Modified | Position | Initial | Modified |
| 13374612 | 73 | A | G | 9 | Thr | Ala |

TABLE D20
Variants of nucleotide sequence CG57094-01

| | Nucleotides | | | Amino Acids | | |
|---|---|---|---|---|---|---|
| Variant | Position | Initial | Modified | Position | Initial | Modified |
| 13377892 | 862 | A | G | 237 | Asn | Asp |
| 13375565 | 947 | C | T | 265 | Thr | Met |
| 13377666 | 1379 | G | A | N/A | N/A | N/A |

### Example E

### Molecular Cloning of NOV26 Variants

For NOV26b, the cDNA coding for the DOMAIN of NOV26a (CG5123-05) from residue 21 to 493 was targeted for “in-frame” cloning by PCR template was based on the previously identified plasmid, when available, or on human cDNA(s). For NOVs 26c-26f, the cDNA coding for the DOMAIN of CG5123-05 from residue 43 to 494 was targeted for “in-frame” cloning by PCR. The PCR template was based on human cDNA(s). NOVs 26g-r, the cDNA coding for the full-length of CG5123-05 from residue 1 to 532 was targeted for the “in-frame” cloning by PCR. The PCR template was based on human cDNA(s).

TABLE E1

Oligonucleotide primers used to clone the target cDNA sequence:

| | | |
|---|---|---|
| NOV26 | | |
| variant | Primers | Sequences |

| | | | |
|---|---|---|---|
| NOV26b | F2 | 5′-AAGCTTGACAGACCTTGGGACCGGGGCCAACACTGG-3′ | (SEQ ID NO:352) |
| | R1 | 5′-CTCGAGAGGAGACATCTCGAAGGGCCACCAAGATGG-3′ | (SEQ ID NO:353) |
| NOV26c-f | F3 | 5′-AAGCTTACTAGGTTTGAGGCGGCCGTGAAGG-3′ | (SEQ ID NO:354) |
| | R1 | 5′-CTCGAGAGGAGACATCTCGAAGGGCCACCAAGATGG-3′ | (SEQ ID NO:355) |
| NOV26g-r | F1 | 5′-AAGCTTCCACCATGTTCCAGTTTCATGCAGGCTCTTGG-3′ | (SEQ ID NO:356) |
| | R2 | 5′-CTCGAGGTTCAGTTTTCTTCTCCTTCTTTGATAG-3′ | (SEQ ID NO:357) |

For downstream cloning purposes, the forward primer includes an in-frame Hind III restriction site and the reverse primer contains an in-frame Xho I restriction site.

Two parallel PCR reactions were set up using a total of 0.5-1.0 ng human pooled cDNAs as template for each reaction. The pool is composed of 5 micrograms of each of the following human tissue cDNAs: adrenal gland, whole brain, amygdala, cerebellum, thalamus, bone marrow, fetal brain, fetal kidney, fetal liver, fetal lung, heart, kidney, liver, lymphoma, Burkitt's Raji cell line, mammary gland, pancreas, pituitary gland, placenta, prostate, salivary gland, skeletal muscle, small Intestine, spleen, stomach, thyroid, trachea, uterus.

When the tissue of expression is known and available, the second PCR was performed using the above primers and 0.5 ng-1.0 ng of one of the following human tissue cDNAs: skeleton muscle, testis, mammary gland, adrenal gland, ovary, colon, normal cerebellum, normal adipose, normal skin, bone marrow, brain amygdala, brain hippocampus, brain substantia nigra, brain thalamus, thyroid, fetal lung, fetal liver, fetal brain, kidney, heart, spleen, uterus, pituitary gland, lymph node, salivary gland, small intestine, prostate, placenta, spinal cord, peripheral blood, trachea, stomach, pancreas, hypothalamus.

The reaction mixtures contained 2 microliters of each of the primers (original concentration: 5 pmol/ul), 1 microliter of 10 mM dNTP (Clontech Laboratories, Palo Alto Calif.) and 1 microliter of 50×Advantage-HF 2 polymerase (Clontech Laboratories) in 50 microliter-reaction volume. The following reaction conditions were used:

PCR condition 1:
a) 96° C. 3 minutes
b) 96° C. 30 seconds denaturation
c) 60° C. 30 seconds, primer annealing
d) 72° C. 6 minutes extension
Repeat steps b-d 15 times
e) 96° C. 15 seconds denaturation
f) 60° C. 30 seconds, primer annealing
g) 72° C. 6 minutes extension
Repeat steps e-g 29 times
e) 72° C. 10 minutes final extension
PCR condition 2:
a) 96° C. 3 minutes
b) 96° C. 15 seconds denaturation
c) 76° C. 30 seconds, primer annealing, reducing the temperature
by 1° C. per cycle
d) 72° C. 4 minutes extension
Repeat steps b-d 34 times
e) 72° C. 10 minutes final extension

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the gene-specific primers in Table .

TABLE E2

Gene-specific Primers

| | | |
|---|---|---|
| NOV26 | | |
| variant | Primers | Sequences |

| | | | |
|---|---|---|---|
| NOV26b | SF1 | GCAGTCTCTGAAGGACATTCTGCAT | (SEQ ID NO:358) |
| | SF2 | TGTTATTCCTGCCATCTTCTCCTCC | (SEQ ID NO:359) |
| | SF3 | ACTTCACTGTCTGAATCGCTTGTCA | (SEQ ID NO:360) |
| | SF4 | TTCTTATGGTCTGCTGACTTCTTCATC | (SEQ ID NO:361) |
| | SF5 | TGACACGCAGCAATTCTTCAACTTT | (SEQ ID NO:362) |
| | SR1 | ACTGCAGAAACTGGAAACGCTGACT | (SEQ ID NO:363) |
| | SR2 | GAGAAGATGGCAGGAATAACAGCG | (SEQ ID NO:364) |
| | SR3 | GTTTACTGTGATTCTATGGAACAATTTGG | (SEQ ID NO:365) |
| | SR4 | CCATAAGAATTTGGAAGTCATTGTCACTAA | (SEQ ID NO:366) |
| | SR5 | TCATAGGTCCATTTTATGAAATTGTCGAG | (SEQ ID NO:367) |
| NOV25e-f | SF1 | CATGTCCTCCTGCAGTCTCATCA | (SEQ ID NO:368) |
| | SF2 | CTTCAACTGCAACCACCTGCATATTCC | (SEQ ID NO:369) |
| | SF3 | GCTGAATTCCTGTAACATCTTCAACA | (SEQ ID NO:370) |
| | SF4 | TTTTCTTATCATTATCACTTTGTTCTGCTCC | (SEQ ID NO:371) |
| | SF5 | ATTCTTCAACTTTCTCAGTCATTGGC | (SEQ ID NO:372) |
| | SR1 | GCTGATGAGACTGCAGGAGGACAT | (SEQ ID NO:373) |
| | SR2 | GAAAAGGTGAAGTCAAGCATGGAGG | (SEQ ID NO:374) |
| | SR3 | TCAGCATTTGACAAGCGATTCAG | (SEQ ID NO:375) |
| | SR4 | AAGAAGTCAGCAGACCATAAGAATTTG | (SEQ ID NO:376) |
| | SR5 | GCTGCGTGTCATAGGTCCATTTT | (SEQ ID NO:377) |
| NOV26g-r | SF1 | CAAAGCAGTCAGCGTTTCCAGTTTCT | (SEQ ID NO:378) |
| | SF2 | CGCTGTTATTCCTGCCATCTTCTC | (SEQ ID NO:379) |
| | SF3 | TCGCTTGTCAAATGCTGAATTCCT | (SEQ ID NO:380) |
| | SF4 | TATCACTTTGTTCTGCTCCTTTCACTT | (SEQ ID NO:381) |
| | SF5 | TCAACATATGCAATCATGGCTTCC | (SEQ ID NO:382) |
| | SR1 | GCAAAATCATCAACATCAACATTGCAG | (SEQ ID NO:383) |
| | SR2 | AGGCGGAGAAACTGACGAATTCTCTAA | (SEQ ID NO:384) |
| | SR3 | ACAAGCGATTCAGACAGTGAAGTTTA | (SEQ ID NO:385) |
| | SR4 | TGATAAGAAAATGATGAAGAAGTCAGC | (SEQ ID NO:386) |
| | SR5 | TGAAAAAGATTATTGAAACTATGCCAA | (SEQ ID NO:387) |

### Example F1

### Angiopoietin-Related Protein (ARP) and Methods of Using ARP

The present invention relates to ARP, a gene surprisingly found to be differentially expressed in clear cell Renal cell carcinoma tissues vs the normal adjacent kidney tissues. Furthermore, this invention demonstrates that ARP is surprisingly differentially expressed in small airway epithelium activated by TNF alpha and IL-1 beta, as well as by lung fibroblasts stimulated by IL-4, IL-9, IL-13 and Interferon gamma relative to untreated lung fibroblasts. Finally, a striking, unexpected upregulation of expression of ARP was observed in Lymphokine-activated killer (LAK) cells treated with the phorbol ester: phorbol- 12, 13-myristate acetate (PMA) in combination with ionomycin, relative to the resting cells.

The present invention discloses a method of using ARP as a clinical marker for staging clear cell Renal cell carcinomas. Furthermore, increased expression of ARP by stimulated LAK cells may play a role in reduced susceptibility of tumor cells to depletion by LAK cells. For the first time, we are disclosing that ARP may be involved with asthma, allergy and emphysema and that regulating ARP by protein therapeutics, antibodies directed against ARP or by small molecule antagonists may alleviate the symptoms of these pulmonary disorders. The invention also discloses a method of treating a pathology treatable by modulating ARP expression, specifically clear cell Renal cell carcinomas.

### Example F2

### Differential Gene Expression in Clear Cell Renal Cell Carcinomas vs Normal Adjacent Tissues

In order to obtain a comprehensive profile of those genes whose expression is modulated in clear cell Renal cell carcinomas, GeneCalling™ technology, described in detail in Shimkets et al. (1999) and in U.S. Pat. No. 5871697, was used to distinguish the gene expression profile of clear cell Renal cell carcinoma tissues with the normal adjacent tissues, obtained from the same patient, during surgical nephrectomy. The tissues were provided to CuraGen from the NDRI under an IRB approved protocol. GeneCalling™ technology relies on Quantitative Expression Analysis to generate the gene expression profile of a given sample and then generates differential expression analysis of pair-wise comparison of these profiles to controls. The comparison in this example is a pool of all tumor tissues vs. a pool of all normal tissues. Polynucleotides exhibiting differential expression were confirmed by conducting a PCR reaction according to the GeneCalling™ protocol, with the addition of a competing unlabelled primer that prevents the amplification from being detected.

Angiopoetin Related Protein (ARP) is overexpressed in 3/5 clear cell renal cell carcinomas, 0/2 papillary renal cell carcinomas and 0/2 uncharacterized renal cell carcinomas (panel 2D). Furthermore ARP is expressed in fetal kidney and renal cell carcinoma-derived cell lines but not in adult kidney (panel 1.3D), an indication of an oncofetal expression pattern often associated with genes involved in kidney development and organogenesis and kidney tumorgenesis.

Data from Panel 4D, indicates that upon immune-stimulation of the airway epithelial cells and lung fibroblasts, ARP is expressed at increased levels. Specifically, we show that expression of ARP in small airway epithelial cells treated with TNF alpha and IL-1 beta is up-regulated ca. 5.4 fold relative to untreated cells. In addition, expression in normal human lung fibroblast cells treated with IL-4, IL-9, IL-9, IL-13 and Interferon gamma is upregulated 7.4, 2, 3.5 and 6.5 fold, respectively, compared to that in resting cells. Finally, expression of ARP in LAK cells treated with PMA/ionomycin is upregulated over 350 fold relative to the expression in resting cells. These data indicate that ARP plays a role in inflammation related to the above cells of the pulmonary system and is thereby implicated as a target for therapeutic intervention by protein and antibody therapeutics as well as small molecule pharmaceuticals. A wholly human antibody directed at ARP, for example, may diminish the symptoms of patients with allergy, asthma or emphysema. A reference (and references therein) for relating airway epithelial cells to asthma and inflammation is: J. Exp. Med. Volume 193, pp339-351 by Michael J. Walter et al. (2001). Another reference for lung fibroblasts and a discussion of asthma and allergy may be found in the review: (abstract included) 1: J Allergy Clin Immunol December 1999;104(6):1139-46 Genetic and environmental interaction in allergy and asthma. Colgate S T Respiratory Cell and Molecular Biology Research Division, Southampton General Hospital, Southampton, United Kingdom.

The upregulation of stimulated LAK cells as seen in Panel 4D-FIG. 4 (greater than 350 fold) was remarkable and surprising. The following references about PMA activation of LAK cells are relevant to the present invention:

1.) Correale P, Procopio A, Celio L, Caraglia M, Genua G, Coppola V, Pepe S, Normanno N, Vecchio I, Palmieri G, et al.

Phorbol 12-myristate 13-acetate induces resistance of human melanoma cells to natural-killer- and lymphokine-activated-killer-mediated cytotoxicity. Cancer Immunol Immunother. 1992;34(4):272-8. PMID: 1371427

2.) Maleci A, Alterman R L, Sundstrom D, Kornblith P L, Moskal J R.

Effect of phorbol esters on the susceptibility of a glioma cell line to lymphokine-activated killer cell activity. J Neurosurg. July 1990;73(l):91-7. PMID: 2352027

3.) Nishimura T, Burakoff S J, Herrmann S H.

Inhibition of lymphokine-activated killer cell-mediated cytotoxicity by phorbol ester. J Immunol. Mar. 15, 1989;142(6):2155-61. PMID: 2646377

Work discussed in 3) indicates that PMA induces down-regulation of LAK cell-mediated cytotoxicity (by inactivation of protein kinase C activity in LAK cells). The exact role of ARP is not known as yet in LAK cells, however, based on the TaqMan data presented in this invention, ARP plays a role in inflammation and may be implicated in the ability of LAK cells to effectively destroy tumor cells as well. Therefore a therapeutic antibody directed against ARP (and thereby preventing ARP from being upregulated), may be therapeutic in treating cancer because of the resulting increased activity of LAK cells.

### Example F3

### Comparing Expression of ARP with Vascular Endothelial Growth Factor (VEGF) Expression

Paradis and coworkers assessed VEGF expression in a large series of renal tumors with a long follow-up, correlated with the usual histo-prognostic factors and survival. Their study revealed that in the group of clear cell RCCs, VEGF expression was positively correlated with both nuclear grade (P=0.05) and size of the tumor (P=0.05). Furthermore, a significant correlation was observed between VEGF expression and microvascular count (P=0.04). Finally, cumulative survival rate was significantly lower in the group of patients with clear cell RCCs expressing VEGF (log rank test, P=0.01). In the Cox model, VEGF expression was a significant independent predictor of outcome, as well as stage and nuclear grade. (Paradis V, Lagha N B, Zeimoura L, Blanchet P, Eschwege P, Ba N, Benoit G, Jardin A, Bedossa P. Expression of vascular endothelial growth factor in renal cell carcinomas. Virchows Arch April 2000;436(4):351-6). The expression profile of VEGF was compared with the expression profile of ARP. As shown in FIG. 3, ARP overexpression is higher and more specific than VEGF, indicating that it could be used as a better clinical marker and that more efficacious and specific therapeutics can be directed at regulating ARP expression. These results also indicate that a treatment that modulates the expression of VEGF and ARP at the same time may achieve synergistic effects. An example of a treatment that can mitigate the effects of the expression of both VEGF and ARP is a bispecific antibody directed both these targets. The bi-specific antibody contemplated to be within the scope of claims for this invention may be an antibody generated by quadroma technology, or by chemical cross-linking of mono-specific antibodies (one directed against VEGF, the other against ARP) or a bi-specific single chain antibody dimer. Formulations of single chain antibodies may include, but not limited to: VL(a)-Linker-VH(a)-Linker-VL(b)-Linker-VH(b). For examples of bispecific antibodies see: U.S. Pat. No. 6,030,792 by Otterness et al., the references therein included here, Multivalent single chain antibodies, U.S. Pat. Nos. 5,892,020, 5,877,291 by Mezes et al., U.S. Pat. No. 6,071,515: Dimer and multimer forms of single chain polypeptides by Mezes et al., and U.S. Pat. No. 6,121,424: Multivalent antigen-binding proteins by Whitlow et al.

### Example F4

### Human PPAR Gamma Angiopoietin Related Protein

Human PPAR gamma angiopoietin related protein is also known as angiopoietin related protein (GenBank ID AF153606), human hepatic angiopoietin-related protein (GeneBank ID AF169312) or angiopoietin-like protein PPl 158 (GeneBank ID AF202636). Recombinant HFARP acts as an apoptosis survival factor for vascular endothelial cells, but does not bind to Tie1 or Tie2 (endothelial-cell tyrosine kinase receptors). These results suggest that HFARP may exert a protective function on endothelial cells through an endocrine action.

(Hepatic expression, synthesis and secretion of a novel fibrinogen/angiopoietin-related protein that prevents endothelial-cell apoptosis. Kim I, Kim H G, Kim H, Kim H H, Park S K, Uhm C S, Lee Z H, Koh G Y Biochem J Mar. 15, 2000; 346 Pt 3:603-10.).

The transcriptional induction of PGAR follows a rapid time course typical of immediate-early genes and occurs in the absence of protein synthesis. The expression of PGAR is predominantly localized to adipose tissues and placenta and is consistently elevated in genetic models of obesity. Hormone-dependent adipocyte differentiation coincides with a dramatic early induction of the PGAR transcript. Alterations in nutrition and leptin administration are found to modulate the PGAR expression in vivo. Taken together, these data suggest a possible role for PGAR in the regulation of systemic lipid metabolism or glucose homeostasis. (Peroxisome proliferator-activated receptor gamma target gene encoding a novel angiopoietin-related protein associated with adipose differentiation. Yoon J C, Chickering T W, Rosen E D, Dussault B, Qin Y, Soukas A, Friedman J M, Holmes W E, Spiegelman B M Mol Cell Biol Jul. 20, 2000 (14):5343-9). The mouse ortholog gene is known as fasting-induced adipose factor FIAF is strongly up-regulated by fasting in white adipose tissue and liver. Moreover, FIAF mRNA is decreased in white adipose tissue of PPARgamma +/− mice. FIAF protein can be detected in various tissues and in blood plasma, suggesting that FIAF has an endocrine function. Its plasma abundance is increased by fasting and decreased by chronic high fat feeding.

| | |
|---|---|
| AF153606.1 Homo sapiens angiopoietin-related protein mRNA | |
| GCGGATCCTCACACGACTGTGATCCGATTCTTTCCAGCGGCTTCTGCAACCAAGCGGGTCTTACCCCCGG | (SEQ ID NO:388) |
| TCCTCCGCGTCTCCAGTCCTCGCACCTGGAACCCCAACGTCCCCGAGAGTCCCCGAATCCCCGCTCCCAG | |
| GCTACCTAAGAGGATGAGCGGTGCTCCGACGGCCGGGGCAGCCCTGATGCTCTGCGCCGCCACCGCCGTG | |
| CTACTGAGCGCTCAGGGCGGACCCGTGCAGTCCAAGTCGCCGCGCTTTGCGTCCTGGGACGAGATGAATG | |
| TCCTGGCGCACGGACTCCTGCAGCTCGGCCAGGGGTGCGCGAACACCGGAGCGCACCCGCAGTCAGCTGA | |
| GCGCGCTGGAGCGCGCCTGAGCGCGTGCGGGTCCGCCTGTCAGGGAACCGAGGGGTCCACCGACCTCCCG | |
| TTAGCCCCTGAGAGCCGGGTGGACCCTGAGGTCCTTCACACCCTGCAGACACAACTCAAGGCTCAGAACA | |
| GCAGGATCCAGCAACTCTTCCACAAGGTGGCCCAGCAGCAGCGGCACCTGGAGAAGCAGCACCTGCGAAT | |
| TCAGCATCTGCAAAGCCAGTTTGGCCTCCTGGACCACAAGCACCTAGACCATGAGGTGGCCAAGCCTGCC | |
| CGAAGAAAGAGGCTGCCCGACATGGCCCAGCCAGTTGACCCGGCTCACAATGTCAGCCGCCTGCACCGGC | |
| TGCCCAGGGATTGCCAGGAGCTGTTCCAGGTTGGGGAGAGGCAGAGTGGACTATTTGAAATCCAGCCTCA | |
| GGGGTCTCCGCCATTTTTGGTGAACTGCAAGATGACCTCAGATGGAGGCTGGACAGTAATTCAGAGGCCC | |
| CACGATGGCTCAGTGGACTTCAACCGGCCCTGGGAAGCCTACAAGGCGGGGTTTGGGGATCCCCACGGCG | |
| AGTTCTGGCTGGGTCTGGAGAAGGTGCATAGCATCACGGGGGACCGCAACAGCCGCCTGGCCGTCCAGCT | |
| GCGGGACTGGCATGOCAACGCCGAGTTGCTGCAGTTCTCCGTGCACCTGGGTGGCGAGGACACGGCCTAT | |
| AGCCTGCAGCTCACTGCACCCGTGGCCGGCCAGCTGGGCGCCACCACCGTCCCACCCAGCGGCCTCTCCG | |
| TACCCTTCTCCACTTGGGACCAGGATCACGACCTCCGCAGGGACAAGAACTGCGCCAAGAGCCTCTCTGG | |
| AGGCTGGTGGTTTGGCACCTGCAGCCATTCCAACCTCAACGGCCAGTACTTCCGCTCCATCCCACAGCAG | |
| CGGCAGAAGCTTAAGAAGGGAATCTTCTGGAAGACCTGGCGGGGCCGCTACTACCCGCTGCAGGCCACCA | |
| CCATGTTGATCCAGCCCATGGCAGCAGAGGCAGCCTCCTAGCGTCCTGGCTGGGCCTGGTCCCAGGCCCA | |
| CGAAAGACGGTGACTCTTGGCTCTGCCCGAGGATGTGGCCAAGACCACGACTGGAGAAGCCCCCTTTCTG | |
| AGTGCAGGGGGGCTGCATGCGTTGCCTCCTGAGATCGAGGCTGCAGGATATGCTCAGACTCTAGAGGCGT | |
| GGACCAAGGGGCATGGAGCTTCACTCCTTGCTGGCCAGGGAGTTGGGGACTCAGAGGGACCACTTGGGGC | |
| CAGCCAGACTGGCCTCAATGGCGGACTCAGTCACATTGACTGACGGGGACCAGGGCTTGTGTGGGTCGAG | |
| ACCGCCCTCATGGTGCTGGTGCTGTTGTGTGTAGGTCCCCTGGGGACACAAGCAGGCGCCAATGGTATCT | |
| GGGCGGAGCTCACAGAGTTCTTGGAATAAAGCAACCTCAGAACAAAAAAAAAAAAAAAAAAGCGGAGCT | |
| CACAGAGTTCTTGGAATAAAAGCAACCTCAGAACAAAAAA | |
| AF169312 hepatic angiopoietin-related protein (ANGPTL2) | |
| TCGCACCTGGAACCCCAACGTCCCCGAGAGTCCCCGAATCCCCGCTCCCAGGCTACCTAAGAGGATGACC | (SEQ ID NO:389) |
| GGTGCTCCGACGGCCGGGGCAGCCCTGATGCTCTGCGCCGCCACCGCCGTGCTACTGAGCGCTCAGGGCG | |
| GACCCGTGCAGTCCAAGTCGCCGCGCTTTGCGTCCTGGGACGAGATGAATGTCCTGGCGCACCGACTCCT | |
| GCAGCTCGGCCAGGGGCTGCGCGAACACGCGGAGCGCACCCGCAGTCAGCTGAGCGCGCTGGAGCGGCGC | |
| CTGAGCGCGTGCGGGTCCGCCTGTCAGGGAACCGAGGGGTCCACCGACCTCCCGTTAGCCCCTGAGAGCC | |
| GGGTGGACCCTGAGGTCCTTCACAGCCTGCAGACACAACTCAAGGCTCAGAACAGCAGGATCCAGCAACT | |
| CTTCCACAAGGTGGCCCAGCAGCAGCGGCACCTGGAGAAGCAGCACCTGCGAATTCAGCATCTGCAAAGC | |
| CAGTTTGGCCTCCTGGACCACAAGCACCTAGACCATGAGGTGGCCAAGCCTGCCCGAAGAAAGAGGCTGC | |
| CCGAGATGGCCCAGCCAGTTGACCCGCCTCACAATGTCAGCCGCCTGCACCGGCTGCCCAGGGATTGCCA | |
| GGAGCTGTTCCAGGTTGGGGAGAGCCAGAGTGGACTATTTGAAATCCACCCTCAGGGGTCTCCGCCATTT | |
| TTGGTGAACTGCAAGATGACCTCAGATGCAGGCTGGACAGTAATTCAGAGGCGCCACGATGGCTCAGTGG | |
| ACTTCAACCGGCCCTGGGAAGCCTACAAGGCGGGGTTTGGGGATCCCCACGGCGAGTTCTGGCTGGGTCT | |
| CGAGAAGGTGCATAGCATCATGGGGGACCGCAACACCCGCCTGGCCGTGCAGCTGCGGGACTGGGATGGC | |
| AACGCCGAGTTGCTGCAGTTCTCCGTGCACCTGGGTGGCGAGGACACGGCCTATAGCCTGCAGTTCACTG | |
| CACCCGTGGCCGGCCAGCTGGGCGCCACCACCGTCCCACCCAGCGGCCTCTCCGTACCCTTCTCCACTTG | |
| GGACCAGGATCACGACCTCCGCAGGGACAAGAACTGCGCCAAGAGCCTCTCTGGAGGCTGGTGGTTTGGC | |
| ACCTGCAGCCATTCCAACCTCAACGGCCACTACTTCCGCTCCATCCCACAGCAGCGGCAGAAGCTTAAGA | |
| AGGGAATCTTCTGGAAGACCTGGCGGGGCCGCTACTACTCGCTGCAGGCCACCACCATGTTGATCCAGCC | |
| CATGGCAGCAGAGGCAGCCTCCTAGCGTCCTGGCTGGGCCTGGTCCCAGCCCCACGAAAGACGGTGACTC | |
| TTGGCTCTGCCCGAGGATGTGGCCGTTCCCTGCCTGGGCAGCGGCTCCAAGGAGGGGCCATCTGGAAACT | |
| TGTGGACAGAGAA | |
| AF2 02636 angiopoietin-Iike protein PP1158 | |
| GGAGAAGAAGCCGAGCTGAGCGGATCCTCACACGACTGTGATCCGATTCTTTCCAGCGGCTTCTGCAACC | (SEQ ID NO:390) |
| AACCGGGTCTTACCCCCGGTCCTCCGCGTCTCCAGTCCTCGCACCTGGAACCCCAACGTCCCCGAGAGTC | |
| CCCGAATCCCCGCTCCCAGGCTACCTAAGAGGATGAGCGGTGCTCCGACGGCCGGGGCAGCCCTGATGCT | |
| CTGCGCCGCCACCGCCGTGCTACTGAGCGCTCAGGGCGGACCCGTGCAGTCCAAGTCGCCGCGCTTTGCG | |
| TCCTCGGACGAGATGAATGTCCTCGCGCACGGACTCCTGCAGCTCGGCCAGGGGCTGCGCGAACACGCGG | |
| AGCGCACCCGCAGTCAGCTGAGCGCGCTGGAGCGGCGCCTGAGCGCGTGCGGGTCCGCCTGTCAGGGAAC | |
| CGAGGGGTCCACCGACCTCCCGTTAGCCCCTGAGAGCCGGGTGGACCCTGAGGTCCTTCACAGCCTGCAG | |
| ACACAACTCAAGGCTCAGAACAGCAGGATCCAGCAACTCTTCCACAAGGTGGCCCAGCAGCAGCGGCACC | |
| TGGAGAAGCAGCACCTGCGAATTCAGCATCTGCAAAGCCAGTTTGGCCTCCTGGACCACAAGCACCTAGA | |
| CCATGAGGTGGCCAAGCCTGCCCGAAGAAAGAGGCTGCCCGAGATGGCCCAGCCAGTTGACCCGGCTCAC | |
| AATGTCAGCCGCCTGCACCGGCTGCCCAGGGATTGCCAGGAGCTGTTCCAGGTTGGGGAGAGCCAGAGTG | |
| GACTATTTGAAATCCAGCCTCAGGGGTCTCCGCCATTTTTGGTGAACTGCAAGATGACCTCAGATGGAGG | |
| CTGGACAGTAATTCAGAGGCGCCACGATGGCTCAGTGGACTTCAACCGGCCCTGGGAAGCCTACAAGGCG | |
| GGGTTTGGGGATCCCCACGGCGAGTTCTGGCTGGGTCTGGAGAAGGTGCATAGCATCACGGGGGACCGCA | |
| ACAGCCGCCTGGCCGTGCAGCTGCGGGACTGGGATGGCAACGCCGAGTTGCTGCAGTTCTCCGTGCACCT | |
| GGGTGGCGAGGACACGGCCTATAGCCTGCAGCTCACTGCACCCGTGGCCGGCCAGCTGGGCGCCACCACC | |
| GTCCCACCCAGCGGCCTCTCCGTACCCTTCTCCACTTGGGACCAGGATCACGACCTCCGCAGGGACAAGA | |
| ACTGCGCCAAGAGCCTCTCTGGAGGCTGGTGGTTTGGCACCTGCAGCCATTCCAACCTCAACGGCCAGTA | |
| CTTCCGCTCCATCCCACAGCAGCGGCAGAAGCTTAAGAAGGGAATCTTCTGGAAGACCTGGCGGGGCCGC | |
| TACTACCCGCTGCAGGCCACCACCATGTTGATCCAGCCCATGGCAGCAGAGGCAGCCTCCTAGCGTCCTG | |
| GCTGGGCCTGGTCCCAGGCCCACGAAAGACGGTGACTCTTGGCTCTGCCCGAGGATGTGGCCGTTCCCTG | |
| CCTGGGCAGGGGCTCCAAGGAGGGGCCATCTGGAAACTTGTGGACAGAGAAGAAGACCACCACTGGAGAA | |
| GCCCCCTTTCTGAGTGCAGGGGGGCTGCATGCGTTGCCTCCTGAGATCGAGGCTGCAGGATATGCTCAGA | |
| CTCTAGAGGCGTGGACCAAGGGGCATCGAGCTTCACTCCTTGCTGGCCAGGGAGTTGGGGACTCAGAGGG | |
| ACCACTTGGGGCCAGCCAGACTGGCCTCAATCGCGGACTCAGTCACATTGACTGACGGGGACCAGGGCTT | |
| GTGTGGGTCGAGAGCGCCCTCATGGTGCTGGTGCTGTTGTGTGTAGGTCCCCTGGGGACACAAGCAGGCG | |
| CCAATGGTATCTGGGCGGCGTCACAGAGTTCTTGGAATAAAAGCAACCTCAGAACACTTAAAAAAAAAAA | |
| AAAAAAAAAAAAAAAAAAAAAAAAAAAAAAAAAAAAAAAAAAAAAAAAAAAAA | |
| NP_057193 angiopoietin related protein | |
| MSGAPTAGAALMLCAATAVLLSAQGGPVQSKSPRFASWDEMNVLAHGLLQLGQGCANTGAHPQSAERAGA | (SEQ ID NO:391) |
| RLSACGSACQGTEGSTDLPLAPESRVDPEVM1SLQTQLKAQNSRIQQLFHKVAQQQRHLEKQHLRIQHLQ | |
| SQFGLLDHKHLDHEVAKPARRKRLPEMAQPVDPAHNVSRLHRLPRDCQELFQVGERQSGLFEIQPQGSPP | |
| FLVNCKMTSDGGWTVIQRRHDGSVDFNRPWEAYKAGFGDPHGEFWLGLEKVHSITGDRNSRLAVQLRDWD | |
| GNAELLQFSVHLGGEDTAYSLQLTAPVAGQLGATTVPPSGLSVPFSTWDQDHDLRRDKNCAKSLSGGWWF | |
| GTCSHSNLNGQYFRSIPQQRQKLKKGIFWKTWRGRYYPLQATTMLIQPMAAEAAS | |
| AAG22490 angiopoietin-like protein PP1158 | |
| MSGAPTAGAALMLCAATAVLLSAQGGPVQSKSPRFASWDEMNVLAHGLLQLGQGLREHAERTRSQLSALE | (SEQ ID NO:392) |
| RRLSACGSACQGTEGSTDLPLAPESRVDPEVLHSLQTQLKAQNSRIQQLFHKVAQQQRHLEKQHLRIQHL | |
| QSQFGLLDHKHLDHEVAKPARRKRLPEMAQPVDPAHNVSRLHRLPRDCQELFQVGERQSGLFEIQPQGSP | |
| PFLVNCKMTSDGGWTVIQRRHDGSVDFNRPWEAYKAGFGDPHGEFWLGLEKVHSITGDRNSRLAVQLRDW | |
| DGNAELLQFSVHLGGEDTAYSLQLTAPVAGQLGATTVPPSGLSVPFSTWDQDHDLRRDKNCAKSLSGGWW | |
| FGTCSHSNLNGQYFRSIPQQRQKLKKGIFWKTWRGRYYPLQATTMLIQPMAAEAAS | |

### Example F5

### GeneCalling Results from Job 36320—all Kidney Cancer vs all Kidney NAT

| ARP—Human angiopoletin-related | | | |
|---|---|---|---|
| protein. (growth factor) | 1 | 1 of 2 | 3 of 9 |

| gbh_af153606 | | Band | Fold | | 67.1 | Set | Visual | | Trap Info | |
|---|---|---|---|---|---|---|---|---|---|---|
| Band ID | Offset | Confirm | Diff. | Sig | Set A | B | Inspection | [symbol] | Score | J1 J2 R1 R2 |

| | | | | | | | | | |
|---|---|---|---|---|---|---|---|---|---|
| d0p0-69.5 | 493 | unconf. | 2.3 | 91 | 108.4 (33.6) | 47.3 (6.1) | [symbol] | [symbol] | |
| q0c0-131.2 (131.2) | 896 | Pass- Complete | 67.3 | 96 | 853.2 (444) | 12.7 (2.6) | [symbol] | [symbol] [symbol] [symbol] | 1 comment |
| p0c0-131.1 | 896 | unconf. | 5.2 | 1 | 398.9 (143.6) | 76.2 (9.7) | [symbol] | [symbol] | |

Results of the GeneCalling job 36320 comparing renal cancers to normal adjacent kidney tissues. Polynucleotides—for e.g. Band ID g0c0-131.2 was identified as being differentially expressed and was confirmed by conducting a PCR reaction according to the GeneCalling™ protocol, with the addition of a competing unlabelled primer that prevents the amplification from being detected and is represented as “Pass complete” in the chart above.

Example F6

TaqMan Panels

[image]

[image]

[image]

### Example F8

### Comparing VEGF and ARP

QEA electrophoresis profile for VEGF (A) and ARP (B) and RTQ-PCR expression profile for VEGF (C) and ARP (D). The differential expression profile of the CG57094 is better than VEGF as demonstrated by GeneCalling and RTQ-PCR.

[image]

### Example F9

### Gene Expression in Tumor Cells Exposed to Serum Starvation, Acidosis and Anoxia and in Brain Tumor Xenograft, RTQ-PCR on HASS Panel v 1.0

The microenvironment within tumors is significantly different from that in normal tissues. Many regions within tumors are transiently or chronically hypoxic due to unbalanced blood supply and significant perfusion heterogeneities. This exacerbates tumor cells' natural tendency to overproduce acids, resulting in very acidic ph values. The hypoxia, trophic limitation and acidity of tumors have important consequences for antitumor therapy and can contribute to the progression of tumors to a more aggressive metastatic phenotype. By subject a set of tumor cell lines to serum starvation, acidosis and anoxia for different time periods, we are modeling the tumor microenviroment.

The HASS panel v 1.0 plates are comprised of 93 cDNA samples and two controls. Specifically, 81 of these samples are derived from cultured human cancer cell lines that had been subjected to serum starvation, acidosis and anoxia for different time periods as well as controls for these treatments, 3 samples of human primary cells, 9 samples of malignant brain cancer (4 medulloblastomas and 5 glioblastomas) and 2 controls. The human cancer cell lines are obtained from ATCC (American Type Culture Collection) and fall into the following tissue groups: breast cancer, prostate cancer, bladder carcinomas, pancreatic cancers and CNS cancer cell lines. These cancer cells are all cultured under standard recommended conditions. The treatments used (serum starvation, acidosis and anoxia) have been previously published in the scientific literature. The primary human cells were obtained from Clonetics (Walkersville, Md.) and were grown in the media and conditions recommended by Clonetics. The malignant brain cancer samples are obtained as part of a collaboration (Henry Ford Cancer Center) and are evaluated by a pathologist prior to CuraGen receiving the samples. RNA was prepared from these samples using the standard procedures. The genomic and chemistry control wells have been described previously.

Results:

CG57094 is expressed at the highest level in U87 cells exposed to hypoxia and acidosis (CT=22.7). The expression of this gene is induced in MCF-7 (breast cancer cell line), T24 (bladder cancer cell line), CaPaN (pancreatic cancer cell line), U87 (CNS cancer), and LnCAP (prostate cancer) cells exposed to low oxygen concentrations. This indicates that expression of this gene may be induced in areas of low oxygen tension in tumors. The gene is also expressed at a higher level in gliomas compared to medulloblastoms and may be used as a marker to distinguish the different kinds of brain cancer. Hence, the therapeutic inhibition of this gene activity, through the use of small molecule drugs or antibodies, might be of utility in the treatment of the above listed cancer types.

[image]

TABLE F9

| | Rel. Expr., % |
|---|---|
| Tissue Name | tm11202t_ag2012_a1 |
| MCF-7 C1 | 0.2 |
| MCF-7 C2 | 0.2 |
| MCF-7 C3 | 0.3 |
| MCF-7 C4 | 0.2 |
| MCF-7 C5 | 0.3 |
| MCF-7 C6 | 0.6 |
| MCF-7 C7 | 6.6 |
| MCF-7 C9 | 10 |
| MCF-7 C10 | 0.4 |
| MCF-7 C11 | 0.1 |
| MCF-7 C12 | 0.5 |
| MCF-7 C13 | 4.5 |
| MCF-7 C15 | 4.2 |
| MCF-7 C16 | 0.6 |
| MCF-7 C17 | 1 |
| T24 D1 | 2.9 |
| T24 D2 | 0.5 |
| T24 D3 | 1.7 |
| T24 D4 | 1.3 |
| T24 D5 | 2.7 |
| T24 D6 | 0.1 |
| T24 D7 | 20.6 |
| T24 D9 | 4.4 |
| T24 D10 | 0.9 |
| T24 D11 | 0.7 |
| T24 D12 | 0.3 |
| T24 D13 | 14.7 |
| T24 D15 | 3.9 |
| T24 D16 | 1.2 |
| T24 D17 | 2.9 |
| CAPaN B1 | 3.8 |
| CAPaN B2 | 1.9 |
| CAPaN B3 | 0.6 |
| CAPaN B4 | 1.3 |
| CAPaN B5 | 1.7 |
| CAPaN B6 | 5.6 |
| CAPaN B7 | 23 |
| CAPaN B8 | 20.3 |
| CAPaN B9 | 59.9 |
| CAPaN B10 | 1.9 |
| CAPaN B11 | 1.9 |
| CAPaN B12 | 4.6 |
| CAPaN B13 | 40.9 |
| CAPaN B14 | 8.1 |
| CAPaN B15 | 5 |
| CAPaN B16 | 8.5 |
| CAPaN B17 | 18.1 |
| U87-MG F1 (B) | 1.8 |
| U87-MG F2 | 1.2 |
| U87-MG F3 | 0 |
| U87-MG F4 | 2.4 |
| U87-MG F5 | 3.9 |
| U87-MG F6 | 0 |
| U87-MG F7 | 59.9 |
| U87-MG F8 | 16.8 |
| U87-MG F9 | 39 |
| U87-MG F10 | 9.3 |
| U87-MG F11 | 0.1 |
| U87-MG F12 | 4.9 |
| U87-MG F13 | 71.1 |
| U87-MG F14 | 30 |
| U87-MG F15 | 100 |
| U87-MG F16 | 7 |
| U87-MG F17 | 14.6 |
| LnCAP A1 | 0.1 |
| LnCAP A2 | 0.1 |
| LnCAP A3 | 0.1 |
| LnCAP A4 | 0.1 |
| LnCAP A5 | 0 |
| LnCAP A6 | 0 |
| LnCAP A7 | 0.9 |
| LnCAP A8 | 0.4 |
| LnCAP A9 | 0.2 |
| LnCAP A10 | 0 |
| LnCAP A11 | 0.1 |
| LnCAP A12 | 0 |
| LnCAP A13 | 0.1 |
| LnCAP A14 | 0.1 |
| LnCAP A15 | 0.1 |
| LnCAP A16 | 0.1 |
| LnCAP A17 | 0.1 |
| Primary Astrocytes | 5.8 |
| Primary Renal Proximal | 14.5 |
| Tubule Epithelial cell A2 | |
| Primary melanocytes A5 | 0.3 |
| 126443 - 341 medullo | 0.2 |
| 126444 - 487 medullo | 2.1 |
| 126445 - 425 medullo | 0 |
| 126446 - 690 medullo | 1.9 |
| 126447 - 54 adult glioma | 2.5 |
| 126448 - 245 adult glioma | 11.7 |
| 126449 - 317 adult glioma | 12.1 |
| 126450 - 212 glioma | 0.8 |
| 126451 - 456 glioma | 2.3 |

### Example F10

### Expression and Therapeutic Relevance in Inflammatory Related Human Diseased and Normal Tissues

CG57094 acts as an apoptosis survival factor for vascular endothelial cells [Kim I, Kim H G, Kim H, Kim H H, Park S K, Uhm C S, Lee Z H, Koh G Y. Hepatic expression, synthesis and secretion of a novel fibrinogen/angiopoietin-related protein that prevents endothelial-cell apoptosis. Biochem J. Mar. 15, 2000;346 Pt 3:603-10]. Interestingly that epithelium cells and fibroblasts activated with proinflammatory cytokines as well as LAK cells expressed high levels of CG57094 mRNA. The above results suggest that CG57094 is an important regulator of inflammation. We used RTQ PCR to test expression of CG57094 mRNA in inflammatory tissues represented on AI comprehensive panel.

Description of AI_Comprehensive Panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1 anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

—M=Male

—F=Female

COPD=Chronic obstructive pulmonary disease

Results.

CG57094, Angiopoeitin Related Protein, mRNA is clearly over expressed in tissues form osteoarthritis patients (CT=26-29). In addition ARP is expressed in moderate levels in rheumatoid arthritis, psoriasis, ulcer colitis, asthma, emphysema and Crohn's disease tissues. This indicate that the gene is involved in regulation of inflammation by possible promoting survival potentially harmfully cellular components such as T killer cells. Therefore therapeutic inhibition of this gene product, through the use of small molecule drugs or antibodies, might be of utility in the treatment of the above listed inflammatory diseases.

[image]

### Example F11

### Gene Expression Analysis using CuraChip in Human Tissues from Tumors and from Equivalent Normal Tissues

CuraGen has developed a gene microarray (CuraChip 1.2) for target identification. It provides a high-throughput means of global mRNA expression analyses of CuraGen's collection of cDNA sequences representing the Pharmaceutically Tractable Genome (PTG). This sequence set includes genes which can be developed into protein therapeutics, or used to develop antibody or small molecule therapeutics. CuraChip 1.2 contains ˜11,000 oligos representing approximately 8,500 gene loci, including (but not restricted to) kinases, ion channels, G-protein coupled receptors (GPCRs), nuclear hormone receptors, proteases, transporters, metabolic enzymes, hormones, growth factors, chemokines, cytokines, complement and coagulation factors, and cell surface receptors.

The CuraChip cDNAs were represented as 30-mer oligodeoxyribonucleotides (oligos) on a glass microchip. Hybridization methods using the longer CuraChip oligos are more specific compared to methods using 25-mer oligos. CuraChip oligos were synthesized with a linker, purified to remove truncated oligos (which can influence hybridization strength and specificity), and spotted on a glass slide. Oligo-dT primers were used to generate cRNA probes for hybridization from samples of interest. A biotin-avidin conjugation system was used to detect hybridized probes with a fluorophore-labeled secondary antibody. Gene expression was analyzed using clustering and correlation bioinformatics tools such as Spotfire® (Spotfire, Inc., 212 Elm Street, Somerville, Mass. 02144) and statistical tools such as multivariate analysis (MVA).

Normalization Method used in CuraChip Software

The median fluorescence intensity of each spot and a background for each spot is read on a scale from 0 to 65,000. CuraGen's CuraChip software, developed in-house, has the capability to present the user with either the raw data (median intensities) or normalized data. If normalized data is chosen, the CuraChip software uses the following method to do mean normalization. The normalization is based on each slide/experiment. Suppose we have:

fg_median is the signal/foreground median for each slide/experiment;

bg_median is the background median for each slide/experiment;

original_value is the difference between fg_median and bg_median;

flag is an indicator of a spot's success or failure, where 0 means success and 1 means failure;

raw_fg_mean is the raw foreground mean for each slide/experiment;

raw_bg_mean is the raw background mean for each slide/experiment;

trim_percentage is the trim percentage for each slide/experiment; this could be defined by the user; currently we are using 2% as the trim percentage for each slide/experiment;

nSpots is the number of spots on each slide;

nslides is the number of slides in each experiment;

fg_mean is the trimmed foreground mean for each slide/experiment;

bg_mean is the trimmed background mean for each slide/experiment;

max_fg_mean is a constant among all slides/experiments, currently 2200.0;

normalized_value is the final normalized value;

coeff is the normalization co-efficient;

MAX_VALUE is a constant representing the highest possible fluorescence reading, currently 65,000.

Step 1. Calculate Trimmed Foreground and Background Means

For each slide/experiment, we first calculate the trimmed foreground mean and the trimmed background mean of all spots, suppose nSpots, on each slide. For each spot, if the data is acceptable (flag=0), we calculate the raw foreground mean and background mean by subtracting the background median from the foreground median for each spot. This is designated as a spot's “original value”. (Note: If flag=1, all values are set to 0.)

| original_value = fg_median - bg_median; | |
|---|---|
| if (flag = = 0) | // experiment is successful |
| { | |
| raw_fg_mean = original_value; | |
| raw_bg_mean = bg_median; | |
| } | |
| else | // experiment is failed |

{
raw_fg_mean = 0.0;

raw_bg_mean= 0.0;

}

After that, we remove (trim) the top and bottom 2% of data points from the data set. After the above calculation, we have nSpot number of foreground means and background means for each slide/experiment, and both lists are sorted. Suppose we have the following sorted lists:

| | |
|---|---|
| raw_fg_mean[1], raw_fg_mean[2], ..., | N = 1, nSpots; |
| raw_fg_mean[N]; | |
| raw_bg_mean[1], raw_bg_mean [2], ..., | N = 1, nSpots; |
| raw_bg_mean[N]; | |

then we calculate the trimmed data points for each slide/experiment. Suppose a is the trimmed start data point and b is the trimmed end data point, we have:

a = ceil(nSpots * trim_percentage);
b = floor(nSpots * (1 - trim_percentage);

The “background mean” is calculated from the background medians for the trimmed data set. For the background mean, we simply calculate the average background mean in interval [a, b] then assign to bg_mean:

bg_mean = (raw_bg_mean[a] + raw_bg_{—}
mean[a+1] +...+ raw_bg_mean[b])/(b-

a+1);

The “foreground mean” is calculated from the “original values” (i.e. background-subtracted spot signal medians); only “original values” greater than 500 are used for this calculation (excluding the trimmed top and bottom 2% of the data). Suppose the sum of those foreground means is sum_raw_fg_mean and the amount of those foreground means is k.

fg_mean = sum_raw_fg_mean / k;

For clarity, a snippet code in Java looks like the following,

| | |
|---|---|
| int | k = 0; |
| double | sum_raw_fg_mean = 0.0; |

for (int j = a; j < b; j++) {

if ( raw_fg_mean[j] > 500 ) {

sum_raw_fg_mean = sum_raw_fg_mean +
raw_fg_mean[j];
k++;
}

}

fg_mean = sum_raw_fg_mean / k;

After the calculation of trimmed foreground means and background means for all slides is complete, we start our normalization procedure.

Step 2. Normalize Data

For each slide a normalization coefficient is calculated which compares the foreground mean of the slide to a fixed maximum foreground mean (2200). This coefficient is:

coeff = max_fg_mean / fg_mean;

The normalized value of each spot is then calculated by multiplying the spot's “original value” by the normalization coefficient. Note that if this value is greater than the maximum reading of 65,000, then the value of 65,000 is used as the normalized value. Also note that if a spot's “original value” is less than the background value, the background value is used.

Recall that original_value = fg_median - bg_median
if ( original_value > bg_mean ) {
normalized_value = min(coeff * original_value, MAX_VALUE);
} else {
normalized_value = coeff*bg_mean;
}

The normalized_value for each spot is the final (normalized) value used in the analysis

### Example F12

### Threshhold for CuraChip Data Analysis

A number of control spots are present on CuraChip 1.2 for efficiency calculations and to provide alternative normalization methods. For example, CuraChip 1.2 contains a number of empty or negative control spots, as well as positive control spots containing a dilution series of oligos that detect the highly-expressed genes Ubiquitin and glyceraldehyde-3-phosphate dehydrogenase (GAPD). An analysis of spot signal level was performed using raw data from 67 hybridizations using all oligos. The maximum signal intensity for each oligo across all 67 hybridizations was determined, and the fold-over-background for this maximum signal was calculated (i.e. if the background reading is 20 and the raw spot intensity is 100, then the fold-over-background for that spot is 5×). The negative control or empty spots do occasionally “fire” or give a signal over the background level; however, they do not fire very strongly, with 77.1% of empty spots firing <3× over background and 91.7% <5× (see burgundy bars in figure below). The positive control spots (Ubiquitin and GAPD, the light blue and dark blue bars, respectively) always fired at >100× background. The experimental oligos (Curaoligos, in yellow below) fired over the entire range of intensities, with some at low fold-over-background intensities. Since the negative control spots do fire occasionally at low levels, we have set a suggested threshhold for data analysis at >5× background.

[image]

Results of PTG Chip 1.2:

One hundred seventy-eight samples of RNA from tissues obtained from surgically dissected tumors, non-diseased tissues from the corresponding organs and tumor xenografts grown in nude nu/nu mices were used to generate probes and run on PTG Chip 1.2. An oligo (optg2_{—}0010188) that corresponds to CG57094 on the PTG Chip 1.2 was scrutinized for its expression profile. The statistical analysis identify significant over-expression in a subset of lung tumors compared with corresponding normal lung tissue and strong expression in melanomas and breast cancers, which do not have matched normal tissue

Thus, based upon its profile, the expression of this gene could be of use as a marker for subsets of lung, melanomas and breast cancers, in addition to the subset of Kidney cancers as previously disclosed. In addition, therapeutic inhibition of the activity of the product of this gene, through the use of antibodies or small molecule drugs, may be useful in the therapy of kidney, lung, melanomas and breast cancers that express CG57094 and are dependent on them

ptg2 0010188 Oligo Sequence:
>ATCTGGAAACTTGTGGACAGAGAAGAAGAC (SEQ ID NO:393)

[image] TABLE F12c

| Tissue | Tissue | absolute | Foreground | background |
|---|---|---|---|---|
| Definition | ID | value | Mean | mean |
| G1C4D21B11- | 1 | 133.57 | 2536.51 | 22.17 |
| 01_Lung | | | | |
| cancer(35C) | | | | |
| G1C4D21B11- | 2 | 24.15 | 2733.37 | 20.31 |
| 02_Lung | | | | |
| NAT(36A) | | | | |
| G1C4D21B11- | 3 | 75 | 2933.33 | 21.31 |
| 03_Lung | | | | |
| cancer(35E) | | | | |
| G1C4D21B11- | 4 | 30.62 | 3808.15 | 19.58 |
| 04_Lung | | | | |
| cancer(365) | | | | |
| G1C4D21B11- | 5 | 67.59 | 3824.5 | 21.07 |
| 05_Lung | | | | |
| cancer(368) | | | | |
| G1C4D21B11- | 6 | 23.36 | 2825.08 | 18.76 |
| 06_Lung | | | | |
| cancer(369) | | | | |
| G1C4D21B11- | 7 | 96.15 | 4152.87 | 26.78 |
| 07_Lung | | | | |
| cancer(36E) | | | | |
| G1C4D21B11- | 8 | 44.14 | 3538.73 | 23.55 |
| 08_Lung | | | | |
| NAT(36F) | | | | |
| G1C4D21B11- | 9 | 38.76 | 4143.89 | 21.18 |
| 09_Lung | | | | |
| cancer(370) | | | | |
| G1C4D21B11- | 10 | 18.71 | 2446.38 | 20.81 |
| 10_Lung | | | | |
| cancer(376) | | | | |
| G1C4D21B11- | 11 | 89.32 | 3989.95 | 27.35 |
| 11_Lung | | | | |
| cancer(378) | | | | |
| G1C4D21B11- | 12 | 50.79 | 4136.72 | 36.64 |
| 12_Lung | | | | |
| cancer(37A) | | | | |
| G1C4D21B11- | 13 | 15.33 | 4083.27 | 28.46 |
| 13_Normal | | | | |
| Lung 4 | | | | |
| G1C4D21B11- | 14 | 30.65 | 4235.38 | 25.22 |
| 14_Normal | | | | |
| Lung 5 | | | | |
| G1C4D21B11- | 15 | 70.81 | 3728.44 | 28.62 |
| 15_CuraChip | | | | |
| reference 1 | | | | |
| G1C4D21B11- | 16 | 157.33 | 2915.57 | 20.5 |
| 16_5.Melanoma | | | | |
| G1C4D21B11- | 17 | 217.38 | 2646.56 | 20.29 |
| 17_6.Melanoma | | | | |
| G1C4D21B11- | 18 | 79.79 | 2509.13 | 23.23 |
| 18_Melanoma | | | | |
| (19585) | | | | |
| G1C4D21B11- | 19 | 51.02 | 2759.91 | 24.22 |
| 19_Normal | | | | |
| Lung 1 | | | | |
| G1C4D21B11- | 20 | 128.42 | 3803.04 | 27.08 |
| 20_Lung | | | | |
| cancer(372) | | | | |
| G1C4D21B11- | 21 | 16.91 | 3771.95 | 25.68 |
| 21_Lung | | | | |
| NAT(35D) | | | | |
| G1C4D21B11- | 22 | 55.63 | 2214.53 | 20.77 |
| 22_Lung | | | | |
| NAT(361) | | | | |
| G1C4D21B11- | 23 | 22.08 | 2134.94 | 21.43 |
| 23_{—} | | | | |
| 1.Melanoma | | | | |
| G1C4D21B11- | 24 | 15.95 | 3656.2 | 20.99 |
| 24_Normal | | | | |
| Lung 2 | | | | |
| G1C4D21B11- | 25 | 234.35 | 3295.08 | 24.19 |
| 25_Lung | | | | |
| cancer(374) | | | | |
| G1C4D21B11- | 26 | 30.3 | 3776.14 | 21.32 |
| 26_Lung | | | | |
| cancer(36B) | | | | |
| G1C4D21B11- | 27 | 37.68 | 1543.94 | 26.44 |
| 27_Lung | | | | |
| cancer(362) | | | | |
| G1C4D21B11- | 28 | 145.95 | 1929.4 | 30.01 |
| 28_Lung | | | | |
| cancer(358) | | | | |
| G1C4D21B11- | 29 | 84.73 | 2375.7 | 20.83 |
| 29_{—} | | | | |
| 2.Melanoma | | | | |
| G1C4D21B11- | 30 | 21.6 | 3157.31 | 22.69 |
| 30_Normal | | | | |
| Lung 3 | | | | |
| G1C4D21B11- | 31 | 153.99 | 4614.72 | 32.86 |
| 31_Lung | | | | |
| NAT(375) | | | | |
| G1C4D21B11- | 32 | 242.45 | 2785.76 | 24.74 |
| 32_Lung | | | | |
| cancer(36D) | | | | |
| G1C4D21B11- | 33 | 17.31 | 4348.91 | 34.21 |
| 33_Lung | | | | |
| NAT(363) | | | | |
| G1C4D21B11- | 34 | 21.52 | 3986.34 | 29.19 |
| 34_Lung | | | | |
| cancer(35A) | | | | |
| G1C4D21B11- | 35 | 200.47 | 2189.36 | 20.44 |
| 35_{—} | | | | |
| 4.Melanoma | | | | |
| G1C4E09B12- | 36 | 18.97 | 2957.66 | 9.6 |
| 54_Prostate | | | | |
| cancer(B8B) | | | | |
| G1C4E09B12- | 37 | 17.73 | 4126.76 | 33.25 |
| 55_Prostate | | | | |
| cancer(B88) | | | | |
| G1C4E09B12- | 38 | 24.69 | 3378.81 | 37.92 |
| 56_Prostate | | | | |
| NAT(B93) | | | | |
| G1C4E09B12- | 39 | 26.54 | 3527 | 42.55 |
| 57_Prostate | | | | |
| cancer(B8C) | | | | |
| G1C4E09B12- | 40 | 24.3 | 4105.44 | 45.35 |
| 58_Prostate | | | | |
| cancer(AD5) | | | | |
| G1C4E09B12- | 41 | 21.87 | 4196.5 | 41.71 |
| 59_Prostate | | | | |
| NAT(AD6) | | | | |
| G1C4E09B12- | 42 | 33.21 | 2830.59 | 42.73 |
| 60_Prostate | | | | |
| cancer(AD7) | | | | |
| G1C4E09B12- | 43 | 19.21 | 3404.14 | 29.72 |
| 61_Prostate | | | | |
| NAT(AD8) | | | | |
| G1C4E09B12- | 44 | 20.54 | 3700.09 | 34.54 |
| 62_Prostate | | | | |
| cancer(ADA) | | | | |
| G1C4E09B12- | 45 | 22.51 | 3022.26 | 30.92 |
| 63_Prostate | | | | |
| NAT(AD9) | | | | |
| G1C4E09B12- | 46 | 21.74 | 3084.26 | 30.48 |
| 64_Prostate | | | | |
| cancer(9E7) | | | | |
| G1C4E09B12- | 47 | 13.57 | 3983.11 | 24.56 |
| 66_Prostate | | | | |
| cancer(A0A) | | | | |
| G1C4E09B12- | 48 | 18.23 | 2889.43 | 23.94 |
| 67_Prostate | | | | |
| cancer(9E2) | | | | |
| G1C4E09B12- | 49 | 13.28 | 4473.72 | 23.53 |
| 68_Pancreatic | | | | |
| cancer(9E4) | | | | |
| G1C4E09B12- | 50 | 12.94 | 3443.44 | 20.25 |
| 69_Pancreatic | | | | |
| cancer(9D8) | | | | |
| G1C4E09B12- | 51 | 20.74 | 3819.27 | 17.3 |
| 70_Pancreatic | | | | |
| cancer(9D4) | | | | |
| G1C4E09B12- | 52 | 23.76 | 3287.48 | 24.17 |
| 71_Pancreatic | | | | |
| cancer(9BE) | | | | |
| G1C4E09B12- | 53 | 41.05 | 2358 | 28.92 |
| 73_Pancreatic | | | | |
| NAT(ADB) | | | | |
| G1C4E09B12- | 54 | 28.39 | 2863.88 | 36.96 |
| 74_Pancreatic | | | | |
| NAT(ADC) | | | | |
| G1C4E09B12- | 55 | 21.32 | 3118.81 | 30.22 |
| 76_Pancreatic | | | | |
| NAT(ADD) | | | | |
| G1C4E09B12- | 56 | 18.02 | 3211.96 | 26.31 |
| 77_Pancreatic | | | | |
| NAT(AED) | | | | |
| G1C4E19B13- | 57 | 53.27 | 1984.83 | 48.06 |
| 1_Colon | | | | |
| cancer(8A3) | | | | |
| G1C4E19B13- | 58 | 51.6 | 1682.5 | 39.46 |
| 10_Colon | | | | |
| NAT(8B6) | | | | |
| G1C4E19B13- | 59 | 45.13 | 2378.93 | 48.8 |
| 12_Colon | | | | |
| NAT(9F1) | | | | |
| G1C4E19B13- | 60 | 52.51 | 1931.28 | 46.1 |
| 13_Colon | | | | |
| cancer(9F2) | | | | |
| G1C4E19B13- | 61 | 49.92 | 2029.41 | 46.05 |
| 14_Colon | | | | |
| NAT(A1D) | | | | |
| G1C4E19B13- | 62 | 42.55 | 2278.96 | 44.08 |
| 15_Colon | | | | |
| cancer(9DB) | | | | |
| G1C4E19B13- | 63 | 59.68 | 1674.01 | 45.41 |
| 16_Colon | | | | |
| NAT(A15) | | | | |
| G1C4E19B13- | 64 | 56.64 | 1360.97 | 35.04 |
| 17_Colon | | | | |
| cancer(A14) | | | | |
| G1C4E19B13- | 65 | 58.01 | 1707.6 | 45.03 |
| 18_Colon | | | | |
| NAT(ACB) | | | | |
| G1C4E19B13- | 66 | 53.49 | 1894.33 | 46.06 |
| 19_Colon | | | | |
| cancer(AC0) | | | | |
| G1C4E19B13- | 67 | 53.4 | 1785.56 | 43.34 |
| 2_Colon | | | | |
| cancer(8A4) | | | | |
| G1C4E19B13- | 68 | 53.97 | 1797.75 | 44.1 |
| 20_Colon | | | | |
| NAT(ACD) | | | | |
| G1C4E19B13- | 69 | 49.29 | 2198.75 | 49.26 |
| 21_Colon | | | | |
| cancer(AC4) | | | | |
| G1C4E19B13- | 70 | 52.18 | 1847.84 | 43.83 |
| 22_Colon | | | | |
| NAT(AC2) | | | | |
| G1C4E19B13- | 71 | 48.1 | 1806.35 | 39.49 |
| 23_Colon | | | | |
| cancer(AC1) | | | | |
| G1C4E19B13- | 72 | 42.7 | 2013.34 | 39.08 |
| 24_Colon | | | | |
| NAT(ACC) | | | | |
| G1C4E19B13- | 73 | 68.18 | 1539.46 | 47.71 |
| 25_Colon | | | | |
| cancer(AC3) | | | | |
| G1C4E19B13- | 74 | 55.27 | 1857.03 | 46.65 |
| 26_Breast | | | | |
| cancer(9B7) | | | | |
| G1C4E19B13- | 75 | 71.21 | 1462.79 | 47.35 |
| 27_Breast | | | | |
| NAT(9CF) | | | | |
| G1C4E19B13- | 76 | 49.21 | 2133.12 | 47.71 |
| 28_Breast | | | | |
| cancer(9B6) | | | | |
| G1C4E19B13- | 77 | 47.76 | 2302.99 | 47.48 |
| 29_Breast | | | | |
| cancer(9C7) | | | | |
| G1C4E19B13- | 78 | 47.69 | 2093.72 | 45.39 |
| 3_Colon | | | | |
| cancer(8A6) | | | | |
| G1C4E19B13- | 79 | 66.26 | 1508.35 | 45.43 |
| 30_Breast | | | | |
| NAT(A11) | | | | |
| G1C4E19B13- | 80 | 45.59 | 2246.51 | 46.55 |
| 31_Breast | | | | |
| cancer(A1A) | | | | |
| G1C4E19B13- | 81 | 54.43 | 1881.09 | 46.54 |
| 32_Breast | | | | |
| cancer(9F3) | | | | |
| G1C4E19B13- | 82 | 49.23 | 2174.46 | 48.66 |
| 33_Breast | | | | |
| cancer(9B8) | | | | |
| G1C4E19B13- | 83 | 64.44 | 1670.58 | 48.93 |
| 34_Breast | | | | |
| NAT(9C4) | | | | |
| G1C4E19B13- | 84 | 44.47 | 1168.07 | 23.61 |
| 35_Breast | | | | |
| cancer(9EF) | | | | |
| G1C4E19B13- | 85 | 41.67 | 1506.95 | 28.54 |
| 36_Breast | | | | |
| cancer(9F0) | | | | |
| G1C4E19B13- | 86 | 70.07 | 1016.05 | 32.36 |
| 37_Breast | | | | |
| cancer(9B4) | | | | |
| G1C4E19B13- | 87 | 47.02 | 2526.83 | 47.27 |
| 38_Breast | | | | |
| cancer(9EC) | | | | |
| G1C4E19B13- | 88 | 66.52 | 1594.35 | 48.21 |
| 4_Colon | | | | |
| cancer(8A7) | | | | |
| G1C4E19B13- | 89 | 42.75 | 2091.33 | 40.64 |
| 44_Colon | | | | |
| cancer(8B7) | | | | |
| G1C4E19B13- | 90 | 35.31 | 2533.34 | 40.66 |
| 5_Colon | | | | |
| cancer(8A9) | | | | |
| G1C4E19B13- | 91 | 41.11 | 1638.43 | 30.62 |
| 6_Colon | | | | |
| cancer(8AB) | | | | |
| G1C4E19B13- | 92 | 46.1 | 1975.26 | 41.39 |
| 7_Colon | | | | |
| cancer(8AC) | | | | |
| G1C4E19B13- | 93 | 58.49 | 1851.09 | 49.21 |
| 8_Colon | | | | |
| NAT(8AD) | | | | |
| G1C4E19B13- | 94 | 53.98 | 1920.15 | 47.11 |
| 9_Colon | | | | |
| cancer(8B5) | | | | |
| G1C4E21B14- | 95 | 3.19 | 1393.31 | 2.02 |
| 1_Cervical | | | | |
| cancer(B08) | | | | |
| G1C4E21B14- | 96 | 13.92 | 1400.44 | 8.86 |
| 10_Brain | | | | |
| cancer(9F8) | | | | |
| G1C4E21B14- | 97 | 15.88 | 655.35 | 4.73 |
| 11_Brain | | | | |
| cancer(9C0) | | | | |
| G1C4E21B14- | 98 | 0.66 | 1403.07 | 0.42 |
| 12_Brain | | | | |
| cancer(9F7) | | | | |
| G1C4E21B14- | 99 | 4.74 | 1509.09 | 3.25 |
| 13_Brain | | | | |
| cancer(A00) | | | | |
| G1C4E21B14- | 100 | 0.82 | 1159.94 | 0.43 |
| 14_Brain | | | | |
| NAT(A01) | | | | |
| G1C4E21B14- | 101 | 1.55 | 1019.67 | 0.72 |
| 15_Brain | | | | |
| cancer(9DA) | | | | |
| G1C4E21B14- | 102 | 4.5 | 1352.85 | 2.77 |
| 16_Brain | | | | |
| cancer(9FE) | | | | |
| G1C4E21B14- | 103 | 6.17 | 1237.61 | 3.47 |
| 17_Brain | | | | |
| cancer(9C6) | | | | |
| G1C4E21B14- | 104 | 5.2 | 917.48 | 2.17 |
| 18_Brain | | | | |
| cancer(9F6) | | | | |
| G1C4E21B14- | 105 | 4.36 | 826.9 | 1.64 |
| 2_Cervical | | | | |
| NAT(AEB) | | | | |
| G1C4E21B14- | 106 | 1.86 | 521.75 | 0.44 |
| 21_Bladder | | | | |
| NAT(23954) | | | | |
| G1C4E21B14- | 107 | 3.06 | 1007.77 | 1.4 |
| 22_Urinary | | | | |
| cancer(AF6) | | | | |
| G1C4E21B14- | 108 | 2.29 | 1256.43 | 1.31 |
| 23_Urinary | | | | |
| cancer(B0C) | | | | |
| G1C4E21B14- | 109 | 2.22 | 1219.17 | 1.23 |
| 24_Urinary | | | | |
| cancer(AE4) | | | | |
| G1C4E21B14- | 110 | 2.21 | 1222.48 | 1.23 |
| 25_Urinary | | | | |
| NAT(B20) | | | | |
| G1C4E21B14- | 111 | 2.03 | 1114.91 | 1.03 |
| 26_Urinary | | | | |
| cancer(AE6) | | | | |
| G1C4E21B14- | 112 | 0.23 | 655.35 | 0.07 |
| 27_Urinary | | | | |
| NAT(B04) | | | | |
| G1C4E21B14- | 113 | 6.64 | 543.73 | 1.64 |
| 28_Urinary | | | | |
| cancer(B07) | | | | |
| G1C4E21B14- | 114 | 0.93 | 1247.4 | 0.53 |
| 29_Urinary | | | | |
| NAT(AF8) | | | | |
| G1C4E21B14- | 115 | 6.72 | 1411.18 | 4.31 |
| 3_Cervical | | | | |
| cancer(AFF) | | | | |
| G1C4E21B14- | 116 | 1.13 | 1221.47 | 0.63 |
| 30_Ovarian | | | | |
| cancer(9D7) | | | | |
| G1C4E21B14- | 117 | 2.51 | 1138.73 | 1.3 |
| 31_Urinary | | | | |
| cancer(AF7) | | | | |
| G1C4E21B14- | 118 | 0 | 1298.98 | 0 |
| 32_Ovarian | | | | |
| cancer(9F5) | | | | |
| G1C4E21B14- | 119 | 4.19 | 1134.77 | 2.16 |
| 33_Ovarian | | | | |
| cancer(A05) | | | | |
| G1C4E21B14- | 120 | 0.65 | 505 | 0.15 |
| 34_0varian | | | | |
| cancer(9BC) | | | | |
| G1C4E21B14- | 121 | 2 | 1025.23 | 0.93 |
| 35_Ovarian | | | | |
| cancer(9C2) | | | | |
| G1C4E21B14- | 122 | 2.8 | 1203.34 | 1.53 |
| 36_Ovarian | | | | |
| cancer(9D9) | | | | |
| G1C4E21B14- | 123 | 1.73 | 685.35 | 0.54 |
| 37_Ovarian | | | | |
| NAT(AC7) | | | | |
| G1C4E21B14- | 124 | 2.61 | 716.79 | 0.85 |
| 38_Ovarian | | | | |
| NAT(AC9) | | | | |
| G1C4E21B14- | 125 | 8.33 | 628.62 | 2.38 |
| 39_Ovarian | | | | |
| NAT(ACA) | | | | |
| G1C4E21B14- | 126 | 11.93 | 1293.21 | 7.01 |
| 4_Cervical | | | | |
| NAT(B1E) | | | | |
| G1C4E21B14- | 127 | 4.02 | 542.12 | 0.99 |
| 40_Ovarian | | | | |
| NAT(AC5) | | | | |
| G1C4E21B14- | 128 | 14.43 | 1512.53 | 9.92 |
| 5_Cervical | | | | |
| cancer(B00) | | | | |
| G1C4E21B14- | 129 | 16.96 | 1136.08 | 8.76 |
| 6_Cervical | | | | |
| NAT(AFA) | | | | |
| G1C4E21B14- | 130 | 23.4 | 1782.82 | 18.96 |
| 7_Cervical | | | | |
| cancer(B1F) | | | | |
| G1C4E21B14- | 131 | 7.92 | 655.35 | 2.36 |
| 8_Cervical | | | | |
| NAT(B1C) | | | | |
| G1C4E21B14- | 132 | 7.41 | 1508.5 | 5.08 |
| 9_Brain | | | | |
| cancer(9F9) | | | | |
| G1C4E23B15- | 133 | 103.28 | 2470.88 | 0 |
| 32_Breast | | | | |
| cancer(D34) | | | | |
| G1C4E23B15- | 134 | 0 | 2602.08 | 0 |
| 33_Breast | | | | |
| cancer(D35) | | | | |
| G1C4E23B15- | 135 | 152.74 | 2909.53 | 0 |
| 34_Breast | | | | |
| cancer(D36) | | | | |
| G1C4E23B15- | 136 | 52.81 | 2811.77 | 0.05 |
| 35_Breast | | | | |
| cancer(D37) | | | | |
| G1C4E23B15- | 137 | 8.84 | 2986.78 | 0.38 |
| 36_Breast | | | | |
| cancer(D38) | | | | |
| G1C4E23B15- | 138 | 0.03 | 3026.22 | 0.04 |
| 37_Breast | | | | |
| cancer(D39) | | | | |
| G1C4E23B15- | 139 | 27.92 | 3072.62 | 0.08 |
| 38_Breast | | | | |
| cancer(D3A) | | | | |
| G1C4E23B15- | 140 | 0.86 | 2571.28 | 0.02 |
| 39_Breast | | | | |
| cancer(D3B) | | | | |
| G1C4E23B15- | 141 | 0.41 | 3213.98 | 0.6 |
| 40_Breast | | | | |
| cancer(D3C) | | | | |
| G1C4E23B15- | 142 | 40.41 | 3484.57 | 2.5 |
| 41_Breast | | | | |
| cancer(D3D) | | | | |
| G1C4E23B15- | 143 | 28.26 | 2958.51 | 0.17 |
| 42_Breast | | | | |
| cancer(D3E) | | | | |
| G1C4E23B15- | 144 | 1.41 | 2937.01 | 1.88 |
| 43_Breast | | | | |
| cancer(D3F) | | | | |
| G1C4E23B15- | 145 | 0.96 | 2751.61 | 1.2 |
| 44_Breast | | | | |
| cancer(D40) | | | | |
| G1C4E23B15- | 146 | 0.81 | 2171.59 | 0.8 |
| 45_Breast | | | | |
| cancer(D42) | | | | |
| G1C4E23B15- | 147 | 43.82 | 2962.09 | 4.5 |
| 46_Breast | | | | |
| cancer(D43) | | | | |
| G1C4E23B15- | 148 | 56.05 | 2551.3 | 3.02 |
| 47_Breast | | | | |
| cancer(D44) | | | | |
| G1C4E23B15- | 149 | 28.87 | 2667.3 | 3.59 |
| 48_Breast | | | | |
| cancer(D45) | | | | |
| G1C4E30B16- | 150 | 21.18 | 2804.32 | 0.56 |
| 1_2.SK-MES | | | | |
| G1C4E30B16- | 151 | 0 | 3402.37 | 0 |
| 10_40.HLaC-79 | | | | |
| G1C4E30B16- | 152 | 28.33 | 2562.59 | 0 |
| 11_43.H226 | | | | |
| G1C4E30B16- | 153 | 300.16 | 4221.68 | 0.09 |
| 12_45.HCT-116 | | | | |
| G1C4E30B16- | 154 | 38.67 | 3243.07 | 0 |
| 13_53.IGROV-1 | | | | |
| G1C4E30B16- | 155 | 54.09 | 3253.75 | 0 |
| 14_59.MX-1 | | | | |
| G1C4E30B16- | 156 | 0 | 3249.59 | 0 |
| 15_63.C33A | | | | |
| G1C4E30B16- | 157 | 0.01 | 2333.08 | 0.01 |
| 16_65.Daudi | | | | |
| G1C4E30B16- | 158 | 0.76 | 2727.71 | 0.94 |
| 17_71.MV522 | | | | |
| G1C4E30B16- | 159 | 0 | 2906.49 | 0 |
| 18_76.RWP-2 | | | | |
| G1C4E30B16- | 160 | 7.91 | 2502.53 | 0.01 |
| 19_77.BON | | | | |
| G1C4E30B16- | 161 | 123.89 | 3604.78 | 0 |
| 2_6.MiaPaCa | | | | |
| G1C4E30B16- | 162 | 2.04 | 2357.18 | 2.19 |
| 20_82.H82 | | | | |
| G1C4E30B16- | 163 | 0.1 | 2759.55 | 0.12 |
| 21_86.H69 | | | | |
| G1C4E30B16- | 164 | 0 | 2687.93 | 0 |
| 22_95.Caki-2 | | | | |
| G1C4E30B16- | 165 | 47.91 | 3352.46 | 0.41 |
| 23_100.LNCaP | | | | |
| G1C4E30B16- | 166 | 95.02 | 2593.12 | 0 |
| 24_101.A549 | | | | |
| G1C4E30B16- | 167 | 37.12 | 3970.51 | 0.07 |
| 25_1. DU145 | | | | |
| G1C4E30B16- | 168 | 41.54 | 3230.65 | 0.14 |
| 26_6. OVCAR-3 | | | | |
| G1C4E30B16- | 169 | 0.05 | 3381.64 | 0.07 |
| 27_11. HT-29 | | | | |
| G1C4E30B16- | 170 | 0.15 | 3610.05 | 0.24 |
| 28_13. DLD-2 | | | | |
| G1C4E30B16- | 171 | 9.59 | 3326.73 | 1.78 |
| 29_18. MCF-7 | | | | |
| G1C4E30B16- | 172 | 6.25 | 2464.22 | 0 |
| 3_9.H460 | | | | |
| G1C4E30B16- | 173 | 0 | 2732.11 | 0 |
| 4_15.SW620 | | | | |
| G1C4E30B16- | 174 | 628.79 | 3519.75 | 0 |
| 5_20.SK-OV-3 | | | | |
| G1C4E30B16- | 175 | 24.13 | 3464.04 | 0.04 |
| 6_23.MDA-231 | | | | |
| G1C4E30B16- | 176 | 360.24 | 3801.64 | 0 |
| 7_27.Caki-1 | | | | |
| G1C4E30B16- | 177 | 0 | 2214.23 | 0 |
| 8_31.PC-3 | | | | |
| G1C4E30B16- | 178 | 24.46 | 3237.95 | 0 |
| 9_35.LoVo | | | | |

### Example F13

### Subcloning and Protein Expression

CG57094 encodes a protein consisting of a signal peptide followed by a coil-coil-like domain (required for oligomerization) followed by a fibrinogen-like domain (required for binding to the receptor). Only the mature region of this protein was expressed (removing the signal peptide and substituting it with a IgKappa signal peptide) because the full length sequence with its own signal peptide did not express and secrete sufficient amount. Two recombinant sequences were made, CG57094-02 and CG57094-04 as described in methods, for expression in mammalian system

### Example F14

### Expression of CG57094-04 in Human Embryonic Kidney 293 Cells

A 1143 bp long BglII-XhoI fragment containing the CG57094-04 sequence was subcloned into BamHI-XhoI digested pCEP4/Sec to generate plasmid 789. The resulting plasmid 789 was transfected into 293 cells using the LipofectaminePlus reagent following the manufacturer's instructions (Invitrogen/Gibco). The cell pellet and supernatant were harvested 72h post transfection and examined for CG57094-04 expression by Western blot (reducing conditions) using an anti-V5 antibody. The gel below shows that CG57094-04 is expressed, and a 35 kDa protein is secreted by 293 cells.

The transient 293 transfection was scaled up yielding 6 L conditioned media, from each scale up, providing material for batches 3 and 4.

[image]

### Example F15

### Expression of CG57094-02 in Stable CHO—K1 Cells

A 1143 bp long BglII-XhoI fragment containing the CG57094-02 sequence was subcloned into BamHI-XhoI digested pEE14.4 Sec to generate plasmid 1614. The resulting plasmid 1614 was transfected into CHO—K1 cells using the LipofectaminePlus reagent following the manufacturer's instructions (Invitrogen/Gibco). The cell pellet and supernatant were harvested 72 h post transfection and examined for CG57094-02 expression by Western blot (reducing conditions) using an anti-V5 antibody. The gel below shows that CG57094-02 is expressed, and a 33 kDa protein is secreted by the CHO—K1 cells at transient level.

The culture media was DMEM, 10% FBS, 1× nonessential amino acids.

[image]

MSX resistant clones were selected using the GS system (Lonza Biologicals) The culture media in the selection process was: Glutamin-free DMEM (JRH), 10% dialyzed FBS, 1× GS supplement (JRH), 25 uM MSX (JRH).

A high expressor clone, was selected for scale up in 10 LWave bioreactors. Two reactors were inoculated. 30 L conditioned media was collected from each reactors yielding batches 2 and 3.

The culture media was harvested 120 h after inoculating the Wave bioreactor and examined for CG57094-04 expression by Western blot (reducing conditions) using an anti-V5 antibody. The gel below represents the Western analysis of the sample.

[image]

The protein secreted as the predicted, 45 kDa molecule.

The culture media in the Wave bioreactor is: EX-Cell302 media, 10% dialyzed FBS, 1× GS supplement, 1× HT supplement, 25 uM MSX.

The difference between the observed molecular weight of the secreted molecule in the transient and in the stable cell line scale up conditions is most likely a consequence of the different culture media used in the two production schemes.

### Example F16

### Protein Expression and Purification

CG57094 variant 02 was expressed and purified in the CHO stable cell system. This method yields both full length protein (around 54 Kd) and a proteolityc fragment of 35 Kd, with a ration of about 1:2 full length/fragment. In non reducing conditions (As seen in the western blot), the full length undergoes oligomerization CG57094 variant 04, that has the same protein sequence as 02, was expressed and purified in the 293 transient cell system. More than 90% of the protein is purified as a proteolitic fragment and thefore does not undergo oligomerization.

Procedure

1. Transfected into attached CHO stable cells with Lipofectamine 2000 in Opti-MEM 1. Overlay with DMEM media with 5% FBS after 4 hours.

2. Harvested after 3, 5 and 7 days incubation at 37° C.

Cell Lysis/Protein Recovery

Procedure

1. Centrifuged at 3000 rpm for 10 min and filter with 0.2 um pore size.

Procedure

1. Metal Affinity Chromatography—Pharmacia 50ml and 5 ml Metal Chelate—Running buffer 20 mM phosphate, pH 7.4, 0.5 M NaCl. Wash with 20 mM, 50 mM, and 100 mM Imidazole. Elute with 500 mM Imidazole.

2. HS Cation Exchange Chromatography—Poros HS 1.6 ml column—30 mM Tris-Cl, pH 8.0, 0.05% CHAPS. Elute with 0-2 M NaCl gradient.

3. Dialysis—@ 4° C. using 3,500 MWCO against 20 mM Tris-HCl, pH7.4+150 mM NaCl.

Protein Quality Control

Western Blot Procedure

Antibody name, catalog # and supplier: Anti-V5-HRP Antibody, 46-0708, Invitrogen (Carlsbad, Calif.), S-protein HRP conjugate, 69047, Novagen (Madison, Wis.)

Antibody dilution buffer: PBS/5% milk/0.1% Tween-20

Wash buffer: PBS/0.1% Tween-20

Detection reagents: ECL (Amersham Biosciences Corp., Piscataway, N.J.)

1. The blot was covered with antibody dilution buffer and incubated on a rocker for one hour at room temperature.

2. The blocking solution was replaced with antibody dilution buffer containing the appropriate amount of conjugate, and the blot was incubated on a rocking platform for one hour at room temperature.

3. The antibody solution was decanted, and the blot was washed quickly with two quick rinses of wash buffer. The blot was then covered with wash buffer and incubated on the rocking platform for five minutes, and the wash buffer was decanted. This process was repeated twice for a total of three five-minute washes.

4. The blot was developed using ECL reagents (Amersham Biosciences Corp., Piscataway, N.J.) as per manufacturer instructons and luminescence was then digitized on a Kodak Image Sciences Imaging Station.

### Example F17

### CG57094-02 Batch2, Plasmid #1614 CHO Stable Cell Line

PROTEIN QUALITY CONTROL DATA
Protein Concentration

| by Bradford | by A₂₈₀ Absorbance | Total Protein Batch | |
|---|---|---|---|
| Method (mg/mL) | (mg/mL) | Quantity (mg) | Protein Storage Buffer Composition |
| 0.181 | ND | 2.1 | 20 mM Tris-HCl, pH 7.4 + 150 mM NaCl |

Protein Characterization

| Amino Acid Sequence | | | _N-Terminus _Internal Peptide |
|---|---|---|---|
| Tags Predicted on Purified Protein | | N-terminal: _None _His _V5 x IgK _Melittin | |
| | | C-terminal: _None x His x V5 | |
| Mass Spectroscopy (kd) | ND | Western Blot Analysis (Ab & Ab dilution) | Anti-V5-HRP Antibody (1:5000) |
| | | | S protein HRP conjugate (1:5000) |

Protein Purity

| | | Predicted Size of Protein | Actual Size of Protein | Estimated | Endotoxin | |
|---|---|---|---|---|---|---|
| Gel | Gel | Engineered into Plasmid | Expressed from Plasmid | Purity | Level | Sterile |
| Composition | Staining | (including tags) (kd) | (including tags) (kd) | (≧ %) | (≦ EU/mg) | Filtered |
| 4-20% Tris | Coomassie | 43 | 54 | 95 | 202 | x Yes |
| Glycine | Blue | | | | | _No |

[image]

### Example F18

### CG57094-02 B3, Plasmid #1614 CHO Stable Cell Line

PROTEIN QUALITY CONTROL DATA
Protein Concentration

| by Bradford | by A₂₈₀ Absorbance | Total Protein Batch | |
|---|---|---|---|
| Method (mg/mL) | (mg/mL) | Quantity (mg) | Protein Storage Buffer Composition |
| 0.26 | ND | 0.54 | 20 mM Tris-HCl, pH 7.4 + 150 mM NaCl |

Protein Characterization

| Amino Acid Sequence | | | _N-Terminus _Internal Peptide |
|---|---|---|---|
| Tags Predicted on Purified Protein | | N-terminal: _None _His _V5 x IgK _Melittin | |
| | | C-terminal: _None x His x V5 | |
| Mass Spectroscopy (kd) | ND | Western Blot Analysis (Ab & Ab dilution) | Anti-V5-HRP Antibody (1:5000) |
| | | | S protein HRP conjugate (1:5000) |

Protein Purity

| | | Predicted Size of Protein | Actual Size of Protein | Estimated | Endotoxin | |
|---|---|---|---|---|---|---|
| Gel | Gel | Engineered into Plasmid | Expressed from Plasmid | Purity | Level | Sterile |
| Composition | Staining | (including tags) (kd) | (including tags) (kd) | (≧ %) | (≦ EU/mg) | Filtered |
| 4-20% Tris | Coomassie | 43 | 54 | 60 | 7.7 | x Yes |
| Glycine | Blue | | | | | _No |

[image]

### Example F19

### CG57094-04 Batch3, 293 Cell Transient Transfection

Method of Purification

1.Metal Affinity Chromatography—PHARMACIA 50 ml Metal Chelate—20 mM sodium phosphate, pH 7.4,0.5 M NaCl. Wash with 20 mM, 50 mM, and 100 mM Imidazole. Elute with 500 mM Imidazole.

2. Metal Affinity Chromatography—PHARMACIA 5 ml Metal Chelate—20 mM sodium phosphate, pH 7.4, 0.5 M NaCl. Elute against a gradient from 0-500 mM Imidazole.

3. Ion-exchange Chromatography—Poros 50 HS column—Elute against a gradient from 0-1M NaCl in 30 mM Tris-Cl, pH 8.0, 0.05% CHAPS.

4. Dialysis—@ 4° C. using 3,500 MW Cutoff against 20 mM Tris-HCl, pH 7.4+150 mM NaCl

[image]

Example F20

CG57094-04 Batch4 293 Cell Transient Transfection

[image]

As indicated in the Certificate of Analysis for the CG57094-04 protein preparation, during expression and purification , the expressed protein undergoes a non obvious proteolityc cleavage that generate a fragment peptide

The protein sequence of this peptide was determined by N-terminal sequencing of the protein preparation generating a N-terminal sequence of LPEMA QPVDP AHXVS. The sequence was determined by transferring the protein to polyvinylidenedifluoride (PVDF) membranes as described in P. Matsudaira, J. Biol. Chem., 261, 10035-10038 (1987). and then performing automated gas-phase sequencing as described in R. M. Hewick, M. W. Hunkapiller, L. E. Hood, and W. J. Dreyer, J. Biol. Chem., 256, 7990-7997 (1981). The COOH terminus is defined by the tag included in the expression construct (V5 and His peptide) both because the tag is used for purification and because the purified protein is still reactive to the V5 antibodies as shown in the western blot. Therefore the normal COOH terminus of the CG57094 protein is present in the purified protein.

The molecular features ot this proteolitic fragments are specifically different from those of the parental sequence, of CG57094-02 and of NL2, specifically this protein does not undergo oligomerization due to the loss of the Coil-Coil domain while retaining the receptor binding region, the fiubrinogen domain. This results in a peptide that it is easier to express and purify while retaining activity as shown in the Cell Survival Assay with 786-O Cells example. It represent a non-obvious result of the expression construct and cell line used for expression.

| | |
|---|---|
| >CG57094-O4_proteolityc_fragment | |
| LPEMAQPVDPAHNVSRLHRLPRDCQELFQVGERQSGLFEIQPQGSPPFLVNCKMTSDGGWTVIQRRHDGS | (SEQ ID NO:394) |
| VDFNRPWEAYKAGFGDPHGEFWLGLEKVHSITGORNSRLAVQLRDWDGNAELLQFSVHLGGEDTAYSLQL | |
| TAPVAGQLGATTVPPSGLSVPFSTWDQDHDLRRDKNCAKSLSGGWWFGTCSHSNLNGQYFRSIPQQRQKL | |
| KKGIFWKTWRGRYYPLQATTMLIQPMAAEAASLE | |
| >NL2_MET_ORF_ | |
| MSGAPTAGAALMLCAATAVLLSAQGGPVQSKSPRFASWDEMNVLAHGLLQLGQGLREHAERTRSQLSALE | (SEQ ID NO:395) |
| RRLSACGSACQGTEGSTDLPLAPESRVDPEVLHSLQTQLKAQNSRIQQLFHKVAQQQRHLEKQHLRIQHL | |
| QSQFGLLDHKHLDHEVAKPARRKRLPEMAQPVDPAHNVSRLHRLPRDCQELFQVGERQSGLFEIQPQGSP | |
| PFLVNCKMTSDGGWTVIQRRHDGSVDFNRPWEAYKAGFGDPHGEFWLGLEKVHSITGDRNSRLAVQLRDW | |
| DGNAELLQFSVHLGGEDTAYSLQLTAPVAGQLGATTVPPSGLSVPFSTWDQDHDLRRDKNCAKSLSGGWW | |
| FGTCSHSNLNGQYFRSIPQQRQKLKKGIFWKTWRGRYYPLQATTMLIQPMAAEAAS | |
| >0057094-02 | |
| GPVQSKSPRFASWDEMNVLAHGLLQLGQGLREHAERTRSQLSALERRLSACGSACQGTEGSTDLPLAPESRVDPE | (SEQ ID NO:396) |
| VLHSLQTQLKAQNSRIQQLFHKVAQQQRHLEKQHLRIQHLQSQFGLLDHKHLDHEVAKPARRKRLPEMAQPVDPA | |
| HNVSRLHRLPRDCQELFQVGERQSGLFEIQPQGSPPFLVNCKMTSDGGWTVIQRRHDGSVDFNRPWEAYKAGFGD | |
| PHGEFWLGLEKVHSITGDRNSRLAVQLRDWDGNAELLQFSVHLGGEDTAYSLQLTAPVAGQLGATTVPPSGLSVP | |
| FSTWDQDHDLRRDKNCAKSLSGGWWFGTCSHSNLNGQYFRSIPQQRQKLKKGIFWKTWRGRYYPLQATTMLIQPM | |
| AAEAAS | |
| >CG57094-04 | |
| RSGPVQSKSPRFASWDEMNVLAHGLLQLGQGLREHAERTRSQLSALERRLSACGSACQGTEGSTDLPLAPESRVD | (SEQ ID NO:397) |
| PEVLHSLQTQLKAQNSRIQQLFHKVAQQQRHLEKQHLRIQHLQSQFGLLDHKHLDHEVAKPARRKRLPEMAQPVD | |
| PAHNVSRLHRLPRDCQELFQVGERQSGLFEIQPQGSPPFLVNCKMTSDGGWTVIQRRHDGSVDFNRPWEAYKAGF | |
| GDPHGEFWLGLEKVHSITGDRNSRLAVQLRDWDGNAELLQFSVHLGGEDTAYSLQLTAPVAGQLGATTVPPSGLS | |
| VPFSTWDQDHDLRRDKNCAKSLSGGWWFGTCSHSNLNGQYFRSIPQQRQKLKKGIFWKTWRGRYYPLQATTMLIQ | |
| PMAAEAASLE | |

### Example F21

### PE 52a: Cellular Proliferation

CG57094 belongs to the angiopoietin-like family of pro-anti angiogenic factors that either induce or inhibit endothelial cell proliferation. We wanted therefore to test whether our preparation CG57094-02 is able to induce or inhibit endothelial cell proliferation. CG57094 did not inhibit endothelial cell proliferation but at a concentration of 10 μg/ml increased the proliferation of HUVEC and HMVEC but the extent of proliferation was not significant.

### Example F22

### Inhibition of HUVEC Proliferation

BrdU Incorporation in HUVEC cells.

Proliferative activity is measured by treatment of serum-starved cultured cells with CG57094-02 at 1 mcg/ml and 0.1 mcg/ml and measurement of BrDU incorporation during DNA synthesis. HUVEC cells were cultured in DMEM supplemented with 10% fetal bovine serum or 10% calf serum respectively. Cells were grown to confluence at 37° C. in 10% CO₂/air. Cells were then starved in DMEM for 24- 72 h. pCEP4sec or pCEP4sec/CG57094-02 enriched conditioned medium was added (10 μL/100 μL of culture) for 18 h. BrdU (10 μM final concentration) was then added and incubated with the cells for 5 h. BrdU incorporation was assayed according to the manufacturer's specifications (Boehringer Mannheim, Indianapolis, Ind.).

1%FBS plus growth factor stimulated the proliferation of HUVEC cells. In the presence of TNF alpha, which was used as positive control, the proliferation of HUVEC cells was markedly inhibited and was comparable to the level of serum free control. CG57094-02 did not significantly affect the proliferation of these endothelial cells at concentrations of 1 μg and 0.1 μg/ml.

[image]

### Example F23

### Inhibition of HMVEC Proliferation

BrdU Incorporation in HMVEC cells.

Proliferative activity is measured by treatment of serum-starved cultured cells with CG57094-02 at 1 mcg/ml and 0.1 mcg/ml and measurement of BrDU incorporation during DNA synthesis. HMVEC cells were cultured in DMEM supplemented with 10% fetal bovine serum or 10% calf serum respectively. Cells were grown to confluence at 37° C. in 10% CO₂/air. Cells were then starved in DMEM for 24- 72 h. pCEP4sec or pCEP4sec/CG57094-02 enriched conditioned medium was added (10 μL/100 μL of culture) for 18 h. BrdU (10 μM final concentration) was then added and incubated with the cells for 5 h. BrdU incorporation was assayed according to the manufacturer's specifications (Boehringer Mannheim, Indianapolis, Ind.).

1%FBS plus growth factor stimulated the proliferation of HMVEC cells. In the presence of TNF alpha, which was used as positive control, the proliferation of HMVEC cells was markedly inhibited and was comparable to the level of serum free control. CG57094-02 did not significantly affect the proliferation of these endothelial cells at concentrations of 1 μg and 0.1 μg/ml.

[image]

### Example F24

### Inhibition of CPAE Proliferation

BrdU Incorporation in Calf pulmonary arterial endothelial cells (CPAE).

Proliferative activity is measured by treatment of serum-starved cultured cells with CG57094-02 at 1 mcg/ml and 0.1 mcg/ml and measurement of BrDU incorporation during DNA synthesis. CPAE cells were cultured in DMEM supplemented with 10% fetal bovine serum or 10% calf serum respectively. Cells were grown to confluence at 37° C. in 10% CO₂/air. Cells were then starved in DMEM for 24- 72 h. pCEP4sec or pCEP4sec/CG57094-02 enriched conditioned medium was added (10 μL/100 μL of culture) for 18 h. BrdU (10 μM final concentration) was then added and incubated with the cells for 5 h. BrdU incorporation was assayed according to the manufacturer's specifications (Boehringer Mannheim, Indianapolis, Ind.).

1% FBS plus growth factor stimulated the proliferation of CPAE cells. In the presence of TNF alpha, which was used as positive control, the proliferation of CPAE cells was markedly inhibited and was comparable to the level of serum free control. CG57094-02 did not significantly affect the proliferation of these endothelial cells at concentrations of 1 μg and 0.1 μg/ml.

[image]

### Example F25

### BrdU Incorporation in HUVEC Cells

Proliferative activity is measured by treatment of serum-starved cultured cells with CG57094-02 at 10 mcg/ml, 1 mcg/ml, 0.5 mcg/ml, and 0.1 mcg/ml and measurement of BrDU incorporation during DNA synthesis. HUVEC cells were cultured in DMEM supplemented with 10% fetal bovine serum or 10% calf serum respectively. Cells were grown to confluence at 37° C. in 10% CO₂/air. Cells were then starved in DMEM for 24-72 h. pCEP4sec or pCEP4sec/CG57094-02 enriched conditioned medium was added (10 μL/100 μL of culture) for 18 h. BrdU (10 μM final concentration) was then added and incubated with the cells for 5 h. BrdU incorporation was assayed according to the manufacturer's specifications (Boehringer Mannheim, Indianapolis, Ind.). VEGF/bFGF combination at 10 ng/ml was used as positive control.

CG57094 at a concentration of 10 μg/ml increased the proliferation of HUVEC but the extent of proliferation was not significant.

[image]

### Example F26

### BrdU Incorporation in HMVEC Cells

Proliferative activity is measured by treatment of serum-starved cultured cells with CG57094-02 at 10 mcg/ml, 5mcg/ml, 1 mcg/ml and 0.1 mcg/ml and measurement of BrDU incorporation during DNA synthesis. HMVEC cells were cultured in DMEM supplemented with 10% fetal bovine serum or 10% calf serum respectively. Cells were grown to confluence at 37° C. in 10% CO₂/air. Cells were then starved in DMEM for 24-72 h. pCEP4sec or pCEP4sec/CG57094-02 enriched conditioned medium was added (10 μL/100 μL of culture) for 18 h. BrdU (10 μM final concentration) was then added and incubated with the cells for 5 h. BrdU incorporation was assayed according to the manufacturer's specifications (Boehringer Mannheim, Indianapolis, IN).

CG57094 at a concentration of 1 0g/ml increased the proliferation of HMVEC but the extent of proliferation was not significant.

[image]

### Example F27

### PE52: Cellular Survival

CG57094 belongs to the angiopoietin-like family of pro-anti angiogenic factors that either induce or inhibit endothelial cell survial upon cellular stress like starvation. We wanted therefore to test whether our preparation CG57094-02 is able to induce or inhibit endothelial cell survial. CG57094 at a concentration up to 0.01 μg/ml increased the survival of HUVEC and HMVEC but not CPAE in a significant fashion.

Cell Viability assay (WST1 survival Assay). Since CG57094-02 did not induce the potent proliferation of endothelial cells, we tested whether the target gene (CG57094-02) would increase the survival of endothelial cell during starvation. Viability of the cells were measured using Wst-1 assay. The cell lines were chosen on the basis of potential cell types implicated in angiogenesis or cancer neovascularization: HUVEC (human umbilical vein endothelial cells), HMVEC-D (endothelial, dermal capillary) and Calf pulmonary arterial endothelial cells (CPAE). 96 well plates (flat bottom) were coated with 100 μl of attachment factor and incubated at 37° C. for one hour. Attachment factor was aspirated and endothelial cells were plated in a DMEM medium containing 0.1% FBS (no growth factors). After 24 h cells were washed and pCEP4sec or pCEP4sec/CG57094-02 enriched conditioned medium was added (10 μL/100 μL of culture) for 48 h. Purified CG57094-02 protein or conditional media was added again, without changing the medium and further incubated for another 24 h. Wst-1 reagent (10 μl/well) was added and incubated for 45min-1 hour at 37° C. Plates were read at 450 nm absorbance.

### Example F28

In the presence of VEGF/bFGF HUVECs survival of HUVEC cells increased markedly as observed by increase in A450 reading compared to starved cells. Interestingly, CG57094-02 at a concentration of 2.5 μg/ml also increased the viability of HUVEC compared to starved cells. This trend remained the same even at concentrations as low as 0.01 μg/ml of CG57094-02. All of these data suggest that CG57094-02 may be a potent survival factor for endothelial cells. Therefore, inhibition of CG57094-02 activity with a neutralizing monoclonal antibody may inhibit neovascularization of tumors as well as diabetic retinopathies.

[image]

### Example F29

CG57094-02 at 1 μg/ml showed a marked increase in HUVEC cell survival as compared to starved cells, which is consistent with the results shown in FIG. 6. Interestingly, at higher protein concentrations, cells exhibited a decreased viability with the greatest effect seen at the 5 μg/mL concentration.

[image]

### Example F30

Consistent with the result seen on HUVEC cells, the survival of HMVEC-d cells were also enhanced by CG57094-02 at lower concentrations. Interestingly, at higher protein concentrations, cells exhibited a decreased viability with the greatest effect seen at the 0.5 μg/mL concentration.

[image]

### Example F31

VEGF/bFGF increased the survival of CPAE cells as observed by an increase in A450 readings compared to starved cells. Although, CG57094-02 also enhanced the survival of HUVEC and HMVEC-d cells, it had no effect on CPAE cells as measured by Wst-1 reagent.

[image]

### Example F32

### Cell Survival Assay 786-0 Cells

786-0 is a human cell line derived from renal carcinoma and lacks one allele and express a truncated protein (AA 1- 104) from the second allele of the von Hippel-Lindau tumor suppressor gene (VHL). The inactivation of the VHL gene predisposes affected individuals to the human VHL cancer syndrome and is associated with sporadic renal cell carcinomas (RCC) and brain hemangioblastomas. We and other people skilled in the art (Pause A, Lee S, Lonergan K M, Klausner R D. The von Hippel-Lindau tumor suppressor gene is required for cell cycle exit upon serum withdrawal. Proc Natl Acad Sci USA Feb. 3, 1998;95(3):993-8) believe that this cell lines represent a suitable in-vitro model to study tumorogenic mechanisms in renal carcinoma.

Specifically in this example, we wanted to test how treating 786-0 cells with CG57094 purified protein influence their survival in serum withdrawal conditions that would otherwise lead to cell death.

Method: Standard testing method (STM) CV-SUV-001

TABLE F32a
DEFINITIONS

| Abbreviation/Term | Description |
|---|---|
| 786-O | Human Renal Cell |
| | Adenocarcinoma (ATCC) |
| FBS | Fetal bovine serum |
| P/S | Penicillin/Streptomycin |
| PBS | Phosphate Buffered Saline |
| SFM | Serum Free Media |
| BSA | Bovine Serum Albumin |

TABLE F32b
REAGENTS, MATERIALS AND EQUIPMENT

| | | Quantity | | | Stock |
|---|---|---|---|---|---|
| Reagent/Material | Location | Required | | Vendor | Number |
| 96-well flat | TC room | 1 per 2 | | Falcon/Becton- | 353072 |
| bottom plates | | proteins | | Dickenson | 08-772-2C |
| | | | | Fisher Scientific | |
| FBS | CV Freezer | 50 | ml | Gemini | 100-106 |
| | 20:110 | | | | |
| BSA | CV Refrigerator | 50 | ml | Sigma | A-9205 |
| | 4:114 | | | | |
| P/S | CV Freezer | 5 | ml | Gibco-BRL | 15140-122 |
| | 20:110 | | | | |
| Trypsin-EDTA | CV Freezer | 50 | ml | Gibco-BRL | 25200-056 |
| (0.25%) | 4:110 | | | | |
| MTS | Main lab, −20° C. | 20 | μl per | Promega | G3581 |
| | #20:110 | | well | | |
| DMEM | CV Refrigerator | 500 | ml | Mediatech | 10-013-CM |
| | 4:110 | | | | |
| Phosphate Buffered | CV Lab | 10 | ml | Mediatech | 20-031-CV |
| Saline, 7.4 | Chemical Shelf | | | | |

Reagent Preparation

Complete DMEM:

DMEM+10%FBS+1% P/S

Starvation medium:

DMEM+0.5% FBS+1% P/S

Serum Free Media

DMEM+0.1%BSA+1% P/S

Procedures

Procedure Summary:

Cells are plated in the inner sixty wells of a 96-well plate in Complete DMEM. The following day, the cells are washed in SFM and treated with CuraProteins in 0.5% FBS/DMEM. Untreated cells serve as baseline controls. Cells cultured in 10% FBS serve as positive controls. On the third day following treatment, MTS is added to the medium and the cells are incubated for 0.5-4 hrs. The absorbance of the wells is then determined using a microplate absorbance reader.

Day 1:

A. Prepare Cells.

1. Wash a flask of 70-80% confluent cells 1× with PBS.

2. Treat cells for 1 min with 5 ml Trypsin/EDTA per T175 flask until cells can be knocked free from the bottom of the culture flask.

3. After cells have been knocked free, add 5 ml of Complete DMEM to flask.

4. Transfer cell suspension to a 15 ml conical bottom centrifuge tube.

5. Centrifuge cell suspension at 1200 RPM for 5 min at 4° C.

6. Resuspend cells with 10 mls of Complete DMEM.

C. Count viable cells using trypan blue in a hemacytometer.

D. Dilute cells with Complete DMEM to yield 5,000 cells/well, 10 mL per plate needed.

E. For blank wells add 100 μl of Complete DMEM no cells.

F. Incubate at 37° C. in 10% CO₂ humidified incubator over-night.

Day2:

A. View plate for appropriate confluency, viability, and consistency of plating from well to well.

1. Wash plate 2 times with SFM.

B. Add CuraProteins and controls to appropriate wells.

1 For positive controls, add 100 μl Complete DMEM in wells.

2. For negative controls, add 100 μl 0.5% FBS/DMEM in wells.

3. For Buffer controls, add similar amount of buffer solution used in highest concentration protein treatments.

4. For blank wells, add 100 μl Complete DMEM in wells.

C. Incubate at 37° C. in 10% CO₂ humidified incubator for next three days.

Day 5:

A. Visually inspect wells for effects and then add 20 μl MTS to each well.

B. Incubate at 37° C. in 10% CO₂ humidified incubator for 0.5-4 hrs.

C. Read plates on PowerWave spectrophotometer at 490 nm, single wavelength (KC4 program/Protocol/MTS490/ save file in MS EXCEL format).

Results of CV-SUV-001:

The results were assessed by measuring the MTS activity of the cells after 5 days of treatment as described above comparing cell treated with various amount of CG57094, (1) relative to cells without serum stimulation stimulation or stimulated with 0.5% serum (negative controls) and (2) in the last experiment, relative to complete media (positive control). The results are considered positive, if the increase of MTS activity is greater than in the negative controls in a statistically significant fashion. The results below are indicative of the utility of the CG57064, and possibly related polypeptides, in pro-angiogenic therapy and specifically in cardiovascular diseases. The IC50 for the 04 preparations of CG57094 is around 5 μg/ml, for the 02 preparation is below 500 ng/ml and above 100 ng/ml. Considering its overexpression in tumor cells and tumor tissues obtained from kidney, lung, melanomas and breast cancers and the cellular data that revealed how tumor cell survival, especially kidney cancer cell survival, is stimulated by CG57094, inhibiting its activity will have utility in cancer therapy and specifically in inhibiting kidney, lung, melanomas and breast cancers.

The results of this set of experiment are non-obvious in light of the previous art both as disclosed by U.S. Pat. No. 6,455,496 and U.S. Pat. No. 6,074,873.

In these applications the inventors disclosed activity only on endothelial cell that is opposite to what we discovered. In example 10 of U.S. Pat. No. 6,074,873 they disclosed that their NL2 preparation induced endothelial cell apoptosis, the opposite of cell survival. Kim et al. (Kim, I; Kim, H G; Kim, H; Kim, H H; Park, S K; Uhm, C S; Lee, Z H; Koh, G Y. Hepatic expression, synthesis and secretion of a novel fibrinogen/angiopoietin-related protein that prevents endothelial-cell apoptosis. Biochem J 2000 346 Pt 3: 603-610.) disclosed a anti-apoptotic activity only on endothelial cells and with a limited effect (30 and 45% reduction). The activity that we discovered on 786-0 has a range of specific activity less that I microgram/ml and the effect is substantial (500-1000%) that permit to set up a screening assay for, neutralizing antibodies (antibodies that bind to CG57094 and related polypeptides and block their activity).

FIG. 22 shows both preparations of CG57094 protein were able to stimulate the survival of 786-0 cells, compared with controls. The 02 preparation appears to have an higher specific activity.

[image]

| | | av. | st. dev | |
|---|---|---|---|---|
| (−) | | 0.020033333 | 0.00608824 | TTEST against |
| 0.5% FBS | | 0.0217 | 0.00497996 | 0.5% serum |
| CG57094-02 B2 | | | | |
| 1 | ug/ml | 0.383033333 | 0.602524965 | 0.406308204 |
| 3 | ug/ml | 1.030033333 | 0.431413182 | 0.056312197 |
| 6 | ug/ml | 1.3587 | 0.033645208 | 0.000250838 |
| 10 | ug/ml | 1.3397 | 0.123405835 | 0.00303949 |
| 30 | ug/ml | 1.289033333 | 0.015275252 | 5.11734E−05 |
| | buffer | 0.0657 | 0.089515362 | 0.472407116 |
| CG57094-04 B4 | | | | |
| 1 | ug/ml | 0.024033333 | 0.013012814 | 0.723436712 |
| 3 | ug/ml | 0.035366667 | 0.009712535 | 0.12823907 |
| 6 | ug/ml | 0.709033333 | 0.261977734 | 0.044591873 |
| 10 | ug/ml | 1.2697 | 0.160726476 | 0.002005621 |
| 30 | ug/ml | 1.489033333 | 0.024006943 | 8.64534E−05 |
| | buffer | 0.017033333 | 0.013503086 | 0.650072894 |

The survival activity was repeated by both preparations of CG57094 protein. The 02 preparation appears to have a higher specific activity than before.

[image]

| | | av. | st. dev | |
|---|---|---|---|---|
| (−) | | 0.043466667 | 0.004633213 | TTEST against |
| 0.5% FBS | | 0.1413 | 0.013397761 | 0.5% serum |
| CG57094-02 B2 | | | | |
| 1 | ug/ml | 1.039466667 | 0.035275109 | 0.00036726 |
| 3 | ug/ml | 1.1418 | 0.04095119 | 0.000446 |
| 6 | ug/ml | 1.1628 | 0.005567764 | 1.5068E−05 |
| 10 | ug/ml | 1.083466667 | 0.142205251 | 0.0060124 |
| 30 | ug/ml | 1.1618 | 0.02007486 | 8.6897E−05 |
| | buffer | 0.161133333 | 0.05770904 | 0.07027706 |
| CG57094-04 B3 | | | | |
| 1 | ug/ml | 0.204133333 | 0.047056703 | 0.20173704 |
| 3 | ug/ml | 0.369133333 | 0.099651058 | 0.02678674 |
| 6 | ug/ml | 0.648466667 | 0.120238652 | 0.01299048 |
| 10 | ug/ml | 1.0808 | 0.023895606 | 0.00011835 |
| 30 | ug/ml | 1.086466667 | 0.046576103 | 0.00074619 |
| | buffer | 0.1138 | 0.036373067 | 0.05843518 |

The survival activity was repeated using 2 batches of the same preparations of CG57094 protein. Batch 03 of preparation 02 had higher specific activity

[image]

| | | av. | st. dev |
|---|---|---|---|
| (−) | | 0.012433333 | 0.002081666 |
| 0.5% FBS | | 0.028433333 | 0.005773503 |
| complete | | 1.0451 | 0.180357977 |
| CG57094-02 B2 | | | |
| 1 | ng/ml | 0.036766667 | 0.009291573 |
| 10 | ng/ml | 0.033766667 | 0.004618802 |
| 100 | ng/ml | 0.035766667 | 0.016165808 |
| 500 | ng/ml | 0.043433333 | 0.003511885 |
| 1 | ug/ml | 0.0661 | 0.00781025 |
| 10 | ug/ml | 1.064433333 | 0.0306159 |
| buffer high | | 0.017433333 | 0.005686241 |
| buffer mid | | 0.027433333 | 0.013576941 |
| CG57094-02 B3 | | | |
| 1 | ng/ml | 0.027766667 | 0.010503968 |
| 10 | ng/ml | 0.034766667 | 0.005859465 |
| 100 | ng/ml | 0.047433333 | 0.002081666 |
| 500 | ng/ml | 0.9061 | 0.064969223 |
| 1 | ug/ml | 1.103766667 | 0.029143324 |
| 10 | ug/ml | 1.1281 | 0.053113087 |

| | | |
|---|---|---|
| buffer high | 0.029433333 | 0.01106044 |
| buffer mid | 0.0221 | 0 |

### Example F33

### Pe 52a1—Cell Survival Assay of Activated T-Lymphocytes and Macrophages

ARP protein is tested for the ability to prevent apoptosis in activated T-lymphocytes and macrophages since it was shown that these cell types are present in knee synovial samples from patients with knee osteoarthritis [Saito I, Koshino T, Nakashima K, Uesugi M, Saito T. Increased cellular infiltrate in inflammatory synovia of osteoarthritic knees. Osteoarthritis Cartilage. February 2002;10(2):156-62.]. The following methods are used for validation of APR effects on T cells and macrophages: measurement of cell proliferation, relevant cytokine production (IL-2, IL-4, IL-6, TNF-a etc.). In addition early apoptosis markers (Anexin V binding) are tested. The increased cell proliferation and cytokine production indicates positive effects of ARP on cell survival. Decreased Anexin V binding also indicates prevention of apoptosis.

For screening of the therapeutic neutralizing antibody similar tests are used. Criteria for antibody selection are as follows:

1. Binding to ARP (ELISA)

Inhibition of survival T lymphocytes and macrophages induced by ARP in vitro.

### Example F34

### Preparation of Antibodies that Bind CG57094

As described above, inhibiting CG57094 activity has utility in cancer therapy and specifically in inhibiting kidney, lung, melanomas and breast cancers. It is know in the art that antibodies that bind secreted factors like CG57094 can inhibit their activity in a process called neutralization. Specifically, neutralizing monoclonal antibodies that bind VEGF have been shown to inhibit tumor growth acting against tumor-induced angiogenesis () Therefore production of polyclonal and monoclonal antibodies directed against CG57094 has utility in cancer therapy and specifically in inhibiting kidney, lung, melanomas and breast cancers. As opposed to VEGF, that is needed only for tumor induced endothelial cell growth and survival, CG57094 is required for cell growth and survival both by endothelial and tumor cells, therefore inhibition of CG57094 activity could have a more pronounced therapeutic effect.

Because of the non-obvious result from the protein expression that indicates how CG57094-04 generate a proteolitic fragment that encode only the fibrinogen domain, we decided to use that fragment as an antigen for immunization. As discussed the fibrinogen domain is the region that binds the receptor, so antibodies that bind to this region are preferable because they have high possibility to be neutralizing.

Method: Techniques for producing the antibodies are known in the art and are described, for example, in “Antibodies, a Laboratory Manual” Eds Harlow and Lane, Cold Spring Harbor publisher. Both rabbits and mice are suitable for the production of polyclonal antibodies, while mice are also suitable for the production of monoclonal antibodies. Mice where the human immunoglubolin genes have replaced the mouse immunoglubolin genes can be used to produce fully human monoclonal antibodies. These antibodies have better pharmaceutical characteristic, no or minimal antibody directed immune reactions that results in loss of therapeutic efficacy and have been shown to eradicate tumor in animal model (Yang X D, Jia X C, Corvalan J R, Wang P, Davis C G, Jakobovits A Eradication of established tumors by a fully human monoclonal antibody to the epidermal growth factor receptor without concomitant chemotherapy. Cancer Res Mar. 15, 1999;59(6):1236-43). Of particular use in this application are bispecific antibody comprised of an antibody unit specific for VEGF and an antibody unit specific for CG57094. We have disclosed that in tumors, specifically in renal cell carcinomas, there is a high correlation between the expression of VEGF and CG57094. Both protein support tumorogenesis by increasing tumor-induced angiogenesis, so an antibody that block the activity of both proteins at once would have a preferable therapeutic activity. An example is VL(a)-Linker-VH(a)-Linker-VL(b)-Linker-VH(b), where a is an antibody variable region segment directed to VEGF and b is an antibody variable region segment directed to CG57094, or vice versa. Other examples of bispecific antibodies are reviewed by Carter Improving the efficacy of antibody-based cancer therapies. Nat Rev Cancer November 2001; 1(2): 118-29

### Example F35

### Generation of Rabbit Polyclonal Antibodies

Rabbit are immunized with the immunogen emulsified in complete Freund's adjuvant and injected subcutaneously or intraperitoneally or intramuscolar in an amount from 50-1000 micrograms. The immunized rabbits are then boosted 10 to 12 days later with additional immunogen emulsified in the selected adjuvant. Thereafter, for several weeks, the rabbits might also be boosted with additional immunization injections. Serum samples may be periodically obtained from the rabbit by bleeding of the ear for testing ELISA assays to detect the antibodies.

### Example F36

### ELISA Protocol to Determine Binding of the Antibodies

Solution Preparation

Coating Buffer (0.1M Carbonate, pH9.5)

8.4 g. NaHCO3, 3.56 g. Na2CO3, pH to 9.5, and dilute to 1 L. with ddH20

Assay Diluent

Pharmingen #26411E

Protocol

Coat a 96-well high protein binding ELISA plate (Corning Costar #3590) with 50 ul. of protein at a concentration of 5 ug/mL. in coating buffer overnight at 4 degrees.

Following day wash the cells 5× 200-300 ul. of 0.5% Tween-20 in PBS.

Block plates with 200 ul. of assay diluent for at least 1 hour at room temperature.

Dilute antibodies in assay diluent.

Wash plate as in step 2.

Add 50 ul. of each antibody dilution to the proper wells for at least 2 hours at room temp.

Wash plate as in step 2.

Add 50 ul. of secondary antibody and incubate for 1 hour at room temp.

Wash plate as in step 2.

Develop assay with 100 ul. of TMB substrate solution/well. (1:1 ratio of solution A+B) (Pharmingen #2642KK)

Stop reaction with 50 ul. sulfuric acid

Read plate at 450 nm with a correction of 550 nm.

Results:

The CG57094-02 purified protein preparation was able to induce a strong immune reaction as shown by the elisa data in FIGS. 25-26-27. Only the immune serum and not the preimmune serum shows strong reactivity against CG57094-02 coated plates (FIGS. 25-26) while no reactivity was seen against non-coated plates (FIG. 27)

This data indicates that the CG57094-02 purified protein preparation is a good immunogen and can be used to generate antibodies.

[image]

TABLE F36a

| Cr064 | | Preimmune | |
|---|---|---|---|
| serum | OD- | serum | OD- |
| dilutions | blank | dilutions | blank |
| 100 | 0.999 | 100 | 0.021 |
| 1000 | 0.876 | 1000 | 0.004 |
| 2000 | 0.931 | 2000 | 0.003 |
| 4000 | 0.963 | 4000 | 0.002 |
| 8000 | 0.732 | 8000 | 0.002 |
| 10000 | 0.669 | 10000 | 0.002 |
| 20000 | 0.511 | 20000 | 0.001 |
| 100000 | 0.147 | 100000 | 0.001 |
| 200000 | 0.084 | 200000 | 0.001 |
| 1000000 | 0.018 | 1000000 | 0.001 |

[image] TABLE F36b

| Cr064 | | Preimmune | |
|---|---|---|---|
| serum | OD- | serum | OD- |
| dilutions | blank | dilutions | blank |
| 100 | 1.066 | 100 | 0.024 |
| 1000 | 1.127 | 1000 | 0.003 |
| 2000 | 1.054 | 2000 | 0.002 |
| 4000 | 0.993 | 4000 | 0.001 |
| 8000 | 0.720 | 8000 | 0.001 |
| 10000 | 0.714 | 10000 | 0.001 |
| 20000 | 0.536 | 20000 | 0.000 |
| 100000 | 0.153 | 100000 | 0.000 |
| 200000 | 0.088 | 200000 | 0.000 |
| 1000000 | 0.017 | 1000000 | 0.001 |

[image] TABLE F36c

| Cr064 | | Preimmune | |
|---|---|---|---|
| serum | OD- | serum | OD- |
| dilutions | blank | dilutions | blank |
| 100 | 0.290 | 100 | 0.030 |
| 1000 | 0.066 | 1000 | 0.005 |
| 2000 | 0.036 | 2,000 | 0.002 |
| 4000 | 0.021 | 4,000 | 0.002 |
| 8000 | 0.010 | 8,000 | 0.001 |
| 10000 | 0.008 | 10,000 | 0.002 |
| 20000 | 0.004 | 20000 | 0.002 |
| 100000 | 0.001 | 100,000 | 0.001 |
| 200000 | 0.002 | 200,000 | 0.001 |
| 1000000 | 0.001 | 1,000,000 | 0.001 |

### Example F37

### Identification of CG57094 Neutralizing Antibodies

As shown in the Cell Survival Assay for 786-O Cells, purified CG57094 has a survival activity for 786-0 with an IC50 for the 04 preparation around 5 μg/ml and for the 02 preparation below 500 ng/ml and above 100 ng/ml.

As previously discussed, the identification of antibodies, preferably fully human monoclonal antibodies that bind to CG57094 and neutralize its activity, limiting or abolishing its ability to rescue cell from serum withdrawal conditions, would be very beneficial because these antibodies will have therapeutic effect against tumors, specifically against kidney, lung, melanomas and breast cancers. To determine whether an antibody can neutralize CG57094 activity, various amounts of such antibody are added to the Cell Survival Assay for 786-0 Cells as described in the method below. The results are assessed by measuring the MTS activity of the cells after 5 days of treatment as described below comparing cell treated with various amount of the antibody, (1) relative treated with non-binding antibody (negative controls) and (2) relative to serum-starved cells (positive control). The results are considered positive, if the decrease in MTS activity is greater than in the negative controls in a statistically significant fashion.

Antibody that can neutralize the CG57094 activity at least with a molar ratio of 10:1 antibody:CG57094 can be useful as therapeutic, lower molar ratio are preferable.

Method: Standard testing method (STM) CV-ANTSUV-001

TABLE F37a
DEFINITIONS

| Abbreviation/Term | Description |
|---|---|
| 786-O | Human Renal Cell Adenocarcinoma (ATCC) |
| FBS | Fetal bovine serum |
| P/S | Penicillin/Streptomycin |
| PBS | Phosphate Buffered Saline |
| SFM | Serum Free Media |
| BSA | Bovine Serum Albumin |
| Negative antibody | Human isotype matched negative control antibody |

TABLE F37b
REAGENTS, MATERIALS AND EQUIPMENT

| Reagent/ | | Quantity | | | Stock |
|---|---|---|---|---|---|
| Material | Location | Required | | Vendor | Number |
| 96-well | TC room | 1 | per 2 | Falcon/Becton- | 353072 |
| flat | | | proteins | Dickenson | 08-772-2C |
| bottom | | | | Fisher | |
| plates | | | | Scientific | |
| FBS | CV Freezer | 50 | ml | Gemini | 100-106 |
| | 20:110 | | | | |
| BSA | CV Refrig- | 50 | ml | Sigma | A-9205 |
| | erator | | | | |
| | 4:114 | | | | |
| P/S | CV Freezer | 5 | ml | Gibco-BRL | 15140-122 |
| | 20:110 | | | | |
| Trypsin- | CV Freezer | 50 | ml | Gibco-BRL | 25200-056 |
| EDTA | 4:110 | | | | |
| (0.25%) | | | | | |
| MTS | Main lab, | 20 | μl per | Promega | G3581 |
| | −20° C. | | well | | |
| | # 20:110 | | | | |
| DMEM | CV Refrig- | 500 | ml | Mediatech | 10-013-CM |
| | erator | | | | |
| | 4:110 | | | | |
| Phosphate | CV Lab | 10 | ml | Mediatech | 20-031-CV |
| Buffered | Chemical | | | | |
| Saline, | Shelf | | | | |
| 7.4 | | | | | |

Procedures

Procedure Summary:

Cells are plated in the inner sixty wells of a 96-well plate in Complete DMEM. The following day, the cells are washed in SFM and treated with CuraProteins in 0.5% FBS/DMEM. Untreated cells serve as baseline controls. Cells cultured in 10% FBS serve as positive controls. On the third day following treatment, MTS is added to the medium and the cells are incubated for 0.5-4 hrs. The absorbance of the wells is then determined using a microplate absorbance reader.

Day 1:

A. Prepare Cells.

1. Wash a flask of 70-80% confluent cells 1× with PBS.

2. Treat cells for˜1 min with 5 ml Trypsin/EDTA per T175 flask until cells can be knocked free from the bottom of the culture flask.

3. After cells have been knocked free, add 5 ml of Complete DMEM to flask.

4. Transfer cell suspension to a 15 ml conical bottom centrifuge tube.

5. Centrifuge cell suspension at 1200 RPM for 5 min at 4° C.

6. Resuspend cells with 10 mls of Complete DMEM.

C. Count viable cells using trypan blue in a hemacytometer.

D. Dilute cells with Complete DMEM to yield 5,000 cells/well, 10 mL per plate needed.

E. For blank wells add 100 μl of Complete DMEM no cells.

F. Incubate at 37° C. in 10% CO₂ humidified incubator over-night.

Day2:

A. View plate for appropriate confluency, viability, and consistency of plating from well to well.

1. Wash plate 2 times with SFM.

B. Add CuraProteins and controls to appropriate wells.

1. For positive controls, add 100 μl Complete DMEM in wells.

2. For negative controls, add 100 μl 0.5% FBS/DMEM in wells.

3. For Buffer controls, add similar amount of buffer solution used in highest concentration protein treatments.

4. For negative antibody control use 100 μl of negative antibody in 0.5% FBS/DMEM. Also use 100 μl of negative antibody and add appropriate (predetermined) concentration of survival factor. Mix and let stand at room temperature for 10 to 20 minutes for binding, then add 100 μl to each of three wells/treatment.

4. For blank wells, add 100 μl Complete DMEM in wells.

5. In an eppendorf tube add appropriate (pre-determined) concentration of survival factor with 10 μg/ml of experimental antibody, and in a second tube, again with survival factor and 1 μg/ml of experimental antibody. Mix tube and let stand for 10 to 20 minutes for binding, then add 100 μl to each of three wells/treatment.

C. Incubate at 37° C. in 10% CO₂ humidified incubator for next three days.

Day 5:

A. Visually inspect wells for effects and then add 20 μl MTS to each well.

B. Incubate at 37° C. in 10% CO₂ humidified incubator for 0.5-4 hrs.

C. Read plates on PowerWave spectrophotometer at 490 nm, single wavelength (KC4 program / Protocol/MTS490/ save file in MS EXCEL format).

Reagent Preparation

Complete DMEM:

DMEM+10%FBS+1% P/S

Starvation medium:

DMEM+0.5% FBS+1% P/S

Serum Free Media

DMEM+0.1%BSA+1% P/S

### Example F38

### Effects of Neutralizing Antibodies Binding to CG57094-04 (defined as CR064) in Matrigel Plug 786-0 Renal Carcinoma Induced Angiogenesis in Athymic Nude Mice

Purified CG50794-02 and 04 have demonstrated ability to increase survival of endothelial and 786-0 tumor cells in cell culture studies. We hypothesize that neutralizing antibodies against CR064 should inhibit survival of endothelial cell and 786-0 tumor cell in cell culture studies. We hypothesize that these antibodies could offer an antiangiogenic and antitumor effect in a 786-0 driven in vivo model of vessel growth. This activity is not limited to this particular cellular model but should be relevant to the angiogenic reponse by other tumor cell lines, preferably those cell lines that naturally express CG50794 polypepetides.

To evaluate the effects of Cr064 in tumor induced angiogenesis Matrigel plug model using 786-0 human clear cell renal carcinoma. This Matrigel plug assay is designed to provide a quantifiable measure of tumor induced angiogenic response under in vivo conditions as a screen for evaluating the antiangiogenic and antitumor efficacy of CR064. Such a strategy has already been used by Liao et al. to show that a neutralizing antibody against Vascular E-Cadherin inhibited tumor-induce angiogenesis (Liao F, Doody J F, Overholser J, Finnerty B, Bassi R, Wu Y, Dejana E, Kussie P, Bohlen P, Hicklin D J. Selective targeting of angiogenic tumor vasculature by vascular endothelial-cadherin antibody inhibits tumor growth without affecting vascular permeability. Cancer Res 2002 May 1 ;62(9):2567-75). Our antibody will have a preferable activity because it will affect the survival not only of endothelial cells but also of tumor cells.

Histological evaluation will assess the total vascularity of the subcutaneously implanted Matrigel plugs, as well as any antiangiogenic effect by CR064. Efficacy for this antibody in this model will be defined as the inhibition of 786-0 cell induced angiogenesis as measured by the establish histological methods described below.

MATERIALS AND METHODS

| | Test System |
|---|---|
| Species/ | Mice Balb/C Athymic homozygous nude |
| strain: | (nu/nu) |
| Physiological | Normal. |
| state: | |
| Age/weight | ˜6-8 weeks, 18-20 g. |
| range at start | |
| of study: | |
| Number/sex | Total of 25 female mice will be required. |
| of animals: | |
| Identification: | Animals are identified by dots at the base |
| | of tail delineating animal numbers. All the |
| | cages will be labeled with protocol number, |
| | group and animal numbers with appropriate |
| | color codes |
| Randomization: | According to body weight. |
| Justification: | This study is designed to use a minimum |
| | of laboratory animals sufficient to detect |
| | meaningful efficacy results within the |
| | treatment period. |
| Replacement: | Animals will not be replaced during this |
| | study. |

Animal Housing and Environment

| | |
|---|---|
| Housing: | Animals will be housed 5 mice per cage in |
| | polycarbonate microisolation cages, wood |
| | chip bedding and suspended food and sterile |
| | water bottles. The cages conform to the |
| | guidelines cited in the Guide for the Care |
| | and Use of Laboratory Animals and the |
| | applicable Standard Operating Procedures. |
| Acclimation: | Mice will be acclimated for 8 days and given |
| | food and sterile water ad libitum. Animals |
| | will be examined prior to initiation of the |
| | study to assure adequate health and suitability. |
| | Animals that are found to be diseased or |
| | unsuitable will not be assigned to the study. |
| Environmental | During the course of the study, 12-hour |
| conditions: | light/12-hour dark cycle will be maintained. |
| | A nominal temperature range of 20 to 23° C. |
| | with a relative humidity between 30% and 70% |
| | will also be maintained. |
| Food/water and | Harlan Teklad rodent diet and sterile water |
| contaminants: | will be provided ad libitum |

Administration of Cr064 antibodies

| | |
|---|---|
| Route and | Cr064 will be dosed IP at least twice a |
| method of | week. |
| administration: | |
| Justification | This route will be used to evaluate |
| for route of | pharmacologic efficacy in this model. |
| administration: | |
| Administered | 1, 5 and 10 mg/kg |
| dose: | |
| Administered | Adjust by body weight, 20 gram mouse/ |
| volume: | 0.2 mls |
| Identity and | 786-0 human renal clear cell |
| lot number: | adenocarcinoma; batch number P1 5IC |
| Physical | Human Renal Clear Cell Adenocarcinoma |
| description: | |
| Source: | ATCC |
| Characterization/ | ATCC |
| certification: | |
| Storage | |
| conditions: | |
| Stability/ | Long-term storage in liquid nitrogen. Thawed |
| expiration | and cultured for 48 hours before use. |
| date: | Harvested cells are stored at 4° C. during |
| | transfer between the laboratory to the |
| | Specific Pathogen Free Facility |

Experimental Design

Mice will be randomized and groups of 5 will be implanted with Matrigel reconstituted with the required tumor cell lines. A total of 0.5 ml of the suspension will be subcutaneously injected into the right flank of athymic, female, nude mice. Additional will be implanted with Matrigel containing 786-0 renal cell carcinoma (1.0×10⁶ cells). Animals implanted with Matrigel containing 786-0 cells will be dosed with 1, 5 and 10 mg/kg, IP, twice daily. Animals will be monitored for 7 days, sacrificed and the Matrigel plugs will be imaged and harvested for further histological evaluation.

TABLE F38a

| Group | | Number of | Matrigel |
|---|---|---|---|
| Number | Treatment^{a} | Animals | Volume/Mouse |
| 1 | Matrigel Alone | 5 | 0.5 mL/Mouse |
| 2 | Matrigel plus 786-0 | 5 | 0.5 mL/Mouse |
| | cells + vehicle | | |
| 3 | Matrigel plus 786-0 | 5 | 0.5 mL/Mouse |
| | cells, CR064 1.0 | | |
| | mg/kg, | | |
| 4 | Matrigel plus 786-0 | 5 | 0.5 mL/Mouse |
| | cells, CR064 5.0 | | |
| | mg/kg, | | |
| 5 | Matrigel plus 786-0 | 5 | 0.5 mL/Mouse |
| | cells, CR064 10 | | |
| | mg/kg | | |

^{a}Matrigel volume of 0.5 ml will be injected into right flank

Clinical Observations/Signs

Mice will be observed daily for moribundity and mortality approximately 60 minutes ing.

Body Weight

Individual body weights of all mice will be recorded daily, for randomization and

Animals Found Dead or Moribund

If animal dies prior to necropsy (found dead) necropsy and histology data will not be included and tissues will not be collected.

Necropsy

At necropsy, animals will be euthanized by CO₂ asphyxiation. The Matrigel plugs will be exposed through surgical removal of the covering skin flap. Digital images will then be recorded of the matrigel.—The matrigel plug will then be surgically removed, and processed as described below. Cervical dislocation of mice under deep anesthesia will be performed before the final disposal of animals.

Matrigel plugs will be then resected carefully and cut into three parts.

One part will be snap frozen in TissueTek and used for cryocut sections.

One part will be fixed in buffered formalin and then embedded in paraffin for sectioning.

One part will be reserved as a backup. Snap frozen and stored at −80° C.

Macroscopic and Histopathology

Formalin Fixed Matrigel Sections:

Three sections/mouse of 5 to 7 μm in thickness will be cut and stained with hematoxylin and Eosin. Sections will be examined under phase contrast microscope. Representative photomicrographs will be recorded [two frames (10× and 40×)]. Infiltration of endothelial cells and vessels will be recorded.

Vessel Staining by Immunohistochemistry:

Frozen Matrigel plugs will be sectioned (5 μm sections) in a Cryocut microtome. Three independent sections per mouse will be made at different levels and used for staining. Sections will be blocked with BSA (0.1%) and then treated with monoclonal antibody reactive to mouse CD31 conjugated to Phycoerythin (dilutions as recommended by the manufacturer). After thorough washings, sections will be mounted under anti-fading reagent (Vecta Shield) and observed under UV microscope using Red filter. Representative Digital images will be captured (two images at 100×and 200×magnification).

Morphometric Analysis of Vessel Density:

Immunofluorescence images of CD31 staining will be analyzed by Skeletinization program as described by Wild et al (1). Data will be processed to provide mean vessel density, node and length for each group.

Data Analysis and Reporting

Statistical Analysis

Final Report

At the conclusion of the study, the results will be reported in full. This final report will include the experimental design, description of local and systemic effects, body weight, mortality and results of macroscopic and histopathologic findings. The format of all textual reports, including figures, tables, and scanned images will conform to CuraGen standards (CuraStandards). Data presentation will include:

Representative Color Photomicrographs

Digital files (JPEG or TIFF or PDB) for permanent record

1. Wild, R., S. Ramakrishnan, J. Sedgewick, and A. W. Griffioen 2000. Quantitative assessment of angiogenesis and tumor vessel architecture by computer-assisted digital image analysis: effects of VEGF-toxin conjugate on tumor microvessel density Microvasc Res. 59:368-76.

### Example F39

### Efficacy Evaluation of CUR64 Against the 786-0 Human Renal Cell Carcinoma Line Grown as a Xenograft in Nude Mice

Purified CG50794-02 and 04 have demonstrated ability to increase survival of endothelial and 786-0 tumor cells in cell culture studies. We hypothesize that neutralizing antibodies against CR064 should inhibit survival of endothelial cell and 786-0 tumor cell in cell culture studies. We hypothesize that these antibodies could offer an antiangiogenic and antitumor effect in a 786-0 driven in vivo model of tumor xenograft.

The ability of this tumor cell line to produce ectopic tumor xenograft in nude mice is known in the art and it has been used to test the anti-tumor activity of several agents (Plonowski A, Schally A V, Nagy A, Kiaris H, Hebert F, Halmos G Inhibition of metastatic renal cell carcinomas expressing somatostatin receptors by a targeted cytotoxic analogue of somatostatin AN-238. Cancer Res 2000 June 1;60(11):2996-3001)

This activity is not limited to this particular cellular model but should be relevant to other tumor cell lines, preferably those cell lines that naturally express CG50794 polypeptides.

Combination therapy of biological compounds like subcutaneous interferon-alpha (IFN-alpha) and interleukin-2 (IL-2) with intravenous 5-fluorouracil (5-FU) is nowdays standard therapy and achieves some long-term survival benefits in patients with metastatic renal cell carcinoma but it is not curative and affects only a subset of patients. It is therefore necessary to discover new agents that either as single therapy or in combination with 5-FU increase both the overall response rate, long term survival and quality of life.

Therefore we test the efficacy of CR064 antibodies in the 786-0 tumor xenograft alone and in combination with 5-FU. Efficacy for this antibody in this model will be defined as tumor growth delay or growth inhibition as single therapy or combination as measured by the established methods described below.

| | Test System |
|---|---|
| Species/strain: | Mouse/ nu/nu |
| Physiological | Normal |
| state: | |
| Age/weight range | Animals aged 5 to 6 weeks with body |
| at start of study: | weight of approximately 20 g |
| Animal supplier: | Charles River |
| Number/sex | 60/Female |
| of animals: | |
| Identification: | Individually tattooed tails. |
| Randomization: | Animals will be randomized prior to |
| | assignment to treatment groups |
| Justification: | Xenograft tumor models present a well |
| | characterized system for testing of |
| | anti-cancer agents. |
| Replacement: | Animals will not be replaced during |
| | the course of the study. |

Animal Housing and Environment

| | |
|---|---|
| Housing: | Static microisolators. |
| Acclimation: | 1 week. |
| Environmental | 12-hour light cycle at 21-22° C. |
| conditions: | (70-72° F.) and 40%-60% humidity. |
| Food/water and | Irradiated standard rodent diet (NIH31 |
| contaminants: | Modified and Irradiated) consisting of: |
| | 18% protein; 5% fat; and 5% fiber; water |
| | (reverse osmosis, 1 ppm Cl), ad libitum |

Administration of Cr064 antibodies

| | |
|---|---|
| Route and method | Cr064 will be dosed IP at least twice |
| of administration: | a week for at least 3 weeks |
| Justification for route | This route will be used to evaluate |
| of administration: | pharmacologic efficacy in this model. |
| Administered | 1, 5 and 10 mg/kg |
| dose: | |
| Administered | Adjust by body weight, 20 gram mouse/ |
| volume: | 0.2 mls |
| Identity and | 786-0 human renal clear cell |
| lot number: | adenocarcinoma; batch number P1 5IC |
| Physical | Human Renal Clear Cell Adenocarcinoma |
| description: | |
| Source: | ATCC |
| Characterization/ | ATCC |
| certification | |
| Stability/ | Long-term storage in liquid nitrogen. |
| expiration date: | Thawed and cultured for 48 hours before |
| | use. Harvested cells are stored at 4° C. |
| | during transfer between the laboratory to |
| | the Specific Pathogen Free Facility |

Experimental Design

After an acclimation period mice will be subcutaneously implanted with 1×1 mm3 fragments of 786-0 tumors. Animals will be randomized and individually identified. Upon tumors reaching a volume of 60-100 mm³ treatment with will begin. Cr064 antibodies will be administered intraperitoneally at the following doses and schedule (Table 1). Mice will be observed daily, tumors and weight will be recorded twice weekly throughout the study period.

TABLE F39a
Study Design

| Group | Number | | Treatment | Volume |
|---|---|---|---|---|
| Number | of Animals | Treatment | Schedule* | (mL) |
| 1 | 10 | Untreated Control | N/A | N/A |
| | females | | | |
| 2 | 10 | 1 mg/kg, IP | EOD × 3 wk | Based on |
| | females | | | weight |
| 3 | 10 | 5 mg/kg, IP | EOD × 3 wk | Based on |
| | females | | | weight |
| 4 | 10 | 10 mg/kg, IP | EOD × 3 wk | Based on |
| | females | | | weight |
| 5 | 10 | 5-FU, | SID, ×5 | Based on |
| | females | 25 mg/kg, IP | | weight |
| 6 | 10 | 3 + 5 | | Based on |
| | females | | | weight |

TABLE F39b
Study Timeline

| | | 60-100 | | | | |
|---|---|---|---|---|---|---|
| | Day | mm³ | Day | Day | Day | |
| Event | −14 | Tumors | 7 | 14 | 15 | Endpoint |
| Receipt of | X | | | | | |
| animals ^{a} | | | | | | |
| Tumor | | X | | | | |
| Implantation | | | | | | |
| Treatment | | EOD × | | | | |
| | | 3 wk | | | | |
| Body weights | | Daily × | | | 2× wk | |
| | | 14 | | | | |
| Harvest | | | | | X | |
| Tumors | | | | | | |
| Scheduled | | | | | | 2000 mg |
| termination | | | | | | tumors |

Experimental Procedures

Tumor bearing animals will be randomized prior to the start of treatment with. Mice will be monitored daily for body condition and health status. Starting at the point where there is a palpable size mass (60-100 mm³) treatment with CR064 will start. The treatment schedule will be 1, 5 or 10 mg/kg, IP, twice daily for 14 days. Throughout the study the animals will be monitored for tumor twice weekly using calipers. Weights will be recorded daily for the treatment period and twice weekly thereafter.

Tumor volumes will be calculated for all remaining animals as well as body weights. Tumor volumes will be analyzed using the methodology described in the data analysis and reporting section.

Tumor Implantation

Tumors will be harvested from healthy tumor-bearing donor animals. The tissues will be homogenized using standard procedures. Cells will be counted and evaluated for viability using trypan blue. Cells will be suspended in serum free media, and a total of 5×10⁶ cells will be subcutaneously implanted in the flank of mice.

Tumor Measurement and Volume Determination

Tumor growth will be measured and recorded 3 times a week using a caliper. Length and width will be measured for each tumor. Tumor volume will be determined using the following formula:

$$Tumor Weight ( mg ) = \frac{{w}^{2} \times l}{2}$$

Clinical Observations/Signs

Animals will be observed daily for significant clinical signs, moribundity and mortality.

Animals Found Dead or Moribund

Percentage of animal mortality and time to death will be recorded for every animal on the study. Mice may be defined moribund and sacrificed if one or more of the following criteria are met:

1) Body weight loss of 20% or greater in a 2-week period.

2) Tumors that inhibit normal physiological function such as eating, drinking, mobility and ability to urinate and or defecate.

3) Tumors that exceed a maximum dimension of 2000 mg as measured by calipers.

4) Ulcerated tumors, tumor producing a exudates or bleeding.

5) Prolonged diarrhea leading to weight loss.

6) Persistent wheezing and respiratory distress

Animals can also be considered moribund if there is prolonged or excessive pain or distress as defined by clinical observations such as: Prostrate, hunched posture, paralysis/paresis, distended abdomen, ulcerations, abscesses, seizures and/or hemorrhages

Animals Found Dead or Moribund

Any adverse effects or unanticipated deaths will be reported to the veterinarian and to CuraGen Corporation immediately.

Table F40:PE201: Transgenic Mouse Production

Transgenic expression of a human gene in a mouse is a useful tool to help determine the function of the product of the gene in instances where the resulting protein product(s) bind to and activate equivalent receptors leading to conserved biological function. Transgenic mice expressing a human protein can also be used as tools to study the inhibitory or activating properties of antibodies to the human protein in vivo. The production and molecular characterization of the transgenic mice was performed by Xenogen Transgenics (Cranberry, N.J.).

Transgenic mice were produced which express CG57094-02 gene driven by the SAP (serum amyeloid P component (SAP) promoter, a gift of Dr. Yamamura, Institute of Molecular Embryology and Genetics, Kumamoto University School of Medicine, Kumamoto, Japan. The promoter drives expression of the gene to produce protein in the liver with slight expression in the postnatal mouse. Mouse embryonic stem cells were microinjected with linearized DNA consisting of the SAP promoter and the downstream gene which encodes CG57094-02. The CG57094-02 sequence is flanked 5′ by an IgK secretory signal sequence and 3′ by DNA encoding V5/His epitopes. Mouse embryos were implanted, and progeny were analyzed for gene integration

Sharma A, Khoury-Christianson A M, White S P, Dhanial N K, Huang Related Articles, Links W Paulhiac C, Friedman E J, Maniula B N, Kumar R.

High-efficiency synthesis of human alpha-endorphin and magainin in the erythrocytes of transgenic mice: a production system for therapeutic peptides.

Proc Natl Acad Sci USA. Sep. 27, 1994;91(20):9337-41.

Founders (mice which have integrated the gene) were identified by PCR of tail genomic DNA. Sera was drawn from the mouse tail vein at age 4 weeks for serum ELISA to examine protein expression in the circulation for genes for which a secreted product is expected. Serum ELISA was performed using [Curamab/polymab/anti-V5 tag—assay in development] in a two-site format. Serum protein positive mice were bred to obtain lines with relatively homogeneous expression of the protein. These mice can be used for phenotypic analysis or disease modeling to determine the role of the CG57094-02 and functional or PK properties of CR064 mAb.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

What is claimed is:

1. An isolated polypeptide comprising the mature form of an amino acid sequenced selected from the group consisting of SEQ ID NO:2n, wherein n is an integer between 1 and 141.

2. An isolated polypeptide comprising an amino acid sequence selected from the group consisting of SEQ ID NO:2n, wherein n is an integer between 1 and 141.

3. An isolated polypeptide comprising an amino acid sequence which is at least 95% identical to an amino acid sequence selected from the group consisting of SEQ ID NO:2n, wherein n is an integer between 1 and 141.

4. An isolated polypeptide, wherein the polypeptide comprises an amino acid sequence comprising one or more conservative substitutions in the amino acid sequence selected from the group consisting of SEQ ID NO:2n, wherein n is an integer between 1 and 141.

5. The polypeptide of claim 1 wherein said polypeptide is naturally occurring.

6. A composition comprising the polypeptide of claim 1 and a carrier.

7. A kit comprising, in one or more containers, the composition of claim 6.

8. The use of a therapeutic in the manufacture of a medicament for treating a syndrome associated with a human disease, the disease selected from a pathology associated with the polypeptide of claim 1, wherein the therapeutic comprises the polypeptide of claim 1.

9. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide, thereby determining the presence or amount of polypeptide in said sample.

10. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of said polypeptide in the sample of step (a) to the expression of the polypeptide present in a control sample from a second mammalian subject known not to have, or not to be predisposed to, said disease,
wherein an alteration in the level of expression of the polypeptide in the first subject as compared to the control sample indicates the presence of or predisposition to said disease.

11. A method of identifying an agent that binds to the polypeptide of claim 1, the method comprising:
(a) introducing said polypeptide to said agent; and
(b) determining whether said agent binds to said polypeptide.

12. The method of claim 11 wherein the agent is a cellular receptor or a downstream effector.

13. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition in the absence of the substance, the substance is identified as a potential therapeutic agent.

14. A method for screening for a modulator of activity of or of latency or predisposition to a pathology associated with the polypeptide of claim 1, said method comprising:
(a) administering a test compound to a test animal at increased risk for a pathology associated with the polypeptide of claim 1, wherein said test animal recombinantly expresses the polypeptide of claim 1;
(b) measuring the activity of said polypeptide in said test animal after administering the compound of step (a); and
(c) comparing the activity of said polypeptide in said test animal with the activity of said polypeptide in a control animal not administered said polypeptide, wherein a change in the activity of said polypeptide in said test animal relative to said control animal indicates the test compound is a modulator activity of or latency or predisposition to, a pathology associated with the polypeptide of claim 1.

15. The method of claim 14, wherein said test animal is a recombinant test animal that expresses a test protein transgene or expresses said transgene under the control of a promoter at an increased level relative to a wild-type test animal, and wherein said promoter is not the native gene promoter of said transgene.

16. A method for modulating the activity of the polypeptide of claim 1, the method comprising contacting a cell sample expressing the polypeptide of claim 1 with a compound that binds to said polypeptide in an amount sufficient to modulate the activity of the polypeptide.

17. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

18. The method of claim 17, wherein the subject is a human.

19. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO:2n, wherein n is an integer between 1 and 141 or a biologically active fragment thereof.

20. An isolated nucleic acid molecule comprising a nucleic acid sequence selected from the group consisting of SEQ ID NO:2n−1, wherein n is an integer between 1 and 141.

21. The nucleic acid molecule of claim 20, wherein the nucleic acid molecule is naturally occurring.

22. A nucleic acid molecule, wherein the nucleic acid molecule differs by a single nucleotide from a nucleic acid sequence selected from the group consisting of SEQ ID NO: 2n−1, wherein n is an integer between 1 and 141.

23. An isolated nucleic acid molecule encoding the mature form of a polypeptide having an amino acid sequence selected from the group consisting of SEQ ID NO:2n, wherein n is an integer between 1 and 141.

24. An isolated nucleic acid molecule comprising a nucleic acid selected from the group consisting of 2n−1, wherein n is an integer between 1 and 141.

25. The nucleic acid molecule of claim 20, wherein said nucleic acid molecule hybridizes under stringent conditions to the nucleotide sequence selected from the group consisting of SEQ ID NO: 2n−1, wherein n is an integer between 1 and 141, or a complement of said nucleotide sequence.

26. A vector comprising the nucleic acid molecule of claim 20.

27. The vector of claim 26, further comprising a promoter operably linked to said nucleic acid molecule.

28. A cell comprising the vector of claim 26.

29. An antibody that immunospecifically binds to the polypeptide of claim 1.

30. The antibody of claim 29, wherein the antibody is a monoclonal antibody.

31. The antibody of claim 29, wherein the antibody is a humanized antibody.

32. A method for determining the presence or amount of the nucleic acid molecule of claim 20 in a sample, the method comprising:
(a) providing said sample;
(b) introducing said sample to a probe that binds to said nucleic acid molecule;
and
(c) determining the presence or amount of said probe bound to said nucleic acid molecule, thereby determining the presence or amount of the nucleic acid molecule in said sample.

33. The method of claim 32 wherein presence or amount of the nucleic acid molecule is used as a marker for cell or tissue type.

34. The method of claim 33 wherein the cell or tissue type is cancerous.

35. A method for determining the presence of or predisposition to a disease associated with altered levels of expression of the nucleic acid molecule of claim 20 in a first mammalian subject, the method comprising:
a) measuring the level of expression of the nucleic acid in a sample from the first mammalian subject; and
b) comparing the level of expression of said nucleic acid in the sample of step (a) to the level of expression of the nucleic acid present in a control sample from a second mammalian subject known not to have or not be predisposed to, the disease;
wherein an alteration in the level of expression of the nucleic acid in the first subject as compared to the control sample indicates the presence of or predisposition to the disease.

36. A method of producing the polypeptide of claim 1, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 141.

37. The method of claim 36 wherein the cell is a bacterial cell.

38. The method of claim 36 wherein the cell is an insect cell.

39. The method of claim 36 wherein the cell is a yeast cell.

40. The method of claim 36 wherein the cell is a mammalian cell.

41. A method of producing the polypeptide of claim 2, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 141.

42. The method of claim 41 wherein the cell is a bacterial cell.

43. The method of claim 41 wherein the cell is an insect cell.

44. The method of claim 41 wherein the cell is a yeast cell.

45. The method of claim 41 wherein the cell is a mammalian cell.
